SEQUENCE LISTING (500 sequences)

SEQ ID NO: 1 - DNA, 1569 bp, human
caggaagatactgtgttttttcacttgccacccagaaatggaaatggagccacagtatttggtatctcttgctatcgacaaattgaagccaaggcactgaaagtaaggcaagcagatatcaccagagagactgttcagaaaagtgtctgtgttctaagcaagctgcctctgtatggtttacttcaagcaaaacttcaactcattacacatgcatattttgaagagaaggatttttcccaaatttctattctaaaggagctttatgaacatatgaatagttccttgggaggtgcttcattagaaggatcccaagtatatcttggtctgtcacctcgagatcttgtccttcattttcgacacaaggtcttaatcctatttaagctaattcttcttgaaaaaaaggttcttttttatatttctccagtgaataaattggtgggtgcactgatgactgtgttatccctttttccaggcatgattgaacatggtctcagtgactgttctcagtatagaccccggaaaagtatgtctgaagatggtgggcttcaggaaagtaacccatgtgcagatgattttgtttctgcatccactgctgatgtttcacataccaacttgggaactatcaggaaagtcatggcaggaaaccatggagaagatgctgccatgaagactgaggagcctttgttccaagtggaagacagcagcaaagggcaggaacccaatgataccaatcaatatttgaaacctccatctcgcccatctccagattcttcagaaagtgactgggaaactttggatcctagtgtcttagaggaccccaacttgaaagaaagggaacagctgggatcagaccagacaaatttgtttccaaaggactctgtcccctcagagagtcttccaattactgtacaacctcaagctaatacgggacaggtagtcctgataccagggctcatttcgggtttggaagaggatcagtatggcatgcccctggccatcttcacaaagggatatctgtgtttgccttacatggcattgcagcagcatcatcttctctccgatgtcaccgttcgggggtttgttgctggagctactaacatcctttttcgacaacagaaacacctcagtgatgccattgtggaagtagaagaagctctgatccagatccatgatccagaactcaggaagctgcttaacccaaccactgcagacctaaggttcgcagactacctagtgaggcacgtgactgagaatcgggatgacgtcttcctagatggcacgggctgggagggaggtgacgaatggatccgggcccagtttgcggtctacattcatgccctgctggctgccacgctgcaattagttctgttcagaatagtgaacgtggcaaaaaaaattggaaacgtcatggtcacaactagccggaatgttgtacaaacaggaaaagctgttggccagtcagttggaggagctttttccagtgcaaagacagctatgtcttcatggctttccactttcaccacttccacctcccaaagtctcactgagccaccagatgagaagccttga

SEQ ID NO: 2 - DNA, 5748 bp, human
gagaagaaagccagtgcgtctctgggcgcaggggccagtggggctcggaggcacaggcaccccgcgacactccaggttccccgacccacgtccctggcagccccgattatttacagcctcagcagagcacggggcgggggcagaggggcccgcccgggagggctgctacttcttaaaacctctgcgggctgcttagtcacagccccccttgcttgggtgtgtccttcgctcgctccctccctccgtcttaggtcactgttttcaacctcgaataaaaactgcagccaacttccgaggcagcctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccactttgatgagcactagtgctacagcaactgagacagcaaccaagaggcaagaaacctgggattggttttcatggttgtttctaccatcagagtcaaagaatcatcttcacacaacaacacaaatggctggtacgtcttcaaataccatctcagcaggctgggagccaaatgaagaaaatgaagatgaaagagacagacacctcagtttttctggatcaggcattgatgatgatgaagattttatctccagcaccatttcaaccacaccacgggcttttgaccacacaaaacagaaccaggactggacccagtggaacccaagccattcaaatccggaagtgctacttcagacaaccacaaggatgactgatgtagacagaaatggcaccactgcttatgaaggaaactggaacccagaagcacaccctcccctcattcaccatgagcatcatgaggaagaagagaccccacattctacaagcacaatccaggcaactcctagtagtacaacggaagaaacagctacccagaaggaacagtggtttggcaacagatggcatgagggatatcgccaaacacccaaagaagactcccattcgacaacagggacagctgcagcctcagctcataccagccatccaatgcaaggaaggacaacaccaagcccagaggacagttcctggactgatttcttcaacccaatctcacaccccatgggacgaggtcatcaagcaggaagaaggatggatatggactccagtcatagtataacgcttcagcctactgcaaatccaaacacaggtttggtggaagatttggacaggacaggacctctttcaatgacaacgcagcagagtaattctcagagcttctctacatcacatgaaggcttggaagaagataaagaccatccaacaacttctactctgacatcaagcaataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaacagaaaaaaaaaaaaaaaaa

SEQ ID NO: 3 - DNA, 5851 bp, human
cttgcgcggtgtggcggccgatgccgctataaaggcttgttttgctgcagggctcatgctcgggagcgtggttgagcggctggcgcggttgtcctggagcaggggcgcaggaattctgatgtgaaactaacagtctgtgagccctggaacctccactcagagaagatgaaggatatcgacataggaaaagagtatatcatccccagtcctgggtatagaagtgtgagggagagaaccagcacttctgggacgcacagagaccgtgaagattccaagttcaggagaactcgaccgttggaatgccaagatgccttggaaacagcagcccgagccgagggcctctctcttgatgcctccatgcattctcagctcagaatcctggatgaggagcatcccaagggaaagtaccatcatggcttgagtgctctgaagcccatccggactacttccaaacaccagcacccagtggacaatgctgggcttttttcctgtatgactttttcgtggctttcttctctggcccgtgtggcccacaagaagggggagctctcaatggaagacgtgtggtctctgtccaagcacgagtcttctgacgtgaactgcagaagactagagagactgtggcaagaagagctgaatgaagttgggccagacgctgcttccctgcgaagggttgtgtggatcttctgccgcaccaggctcatcctgtccatcgtgtgcctgatgatcacgcagctggctggcttcagtggaccagccttcatggtgaaacacctcttggagtatacccaggcaacagagtctaacctgcagtacagcttgttgttagtgctgggcctcctcctgacggaaatcgtgcggtcttggtcgcttgcactgacttgggcattgaattaccgaaccggtgtccgcttgcggggggccatcctaaccatggcatttaagaagatccttaagttaaagaacattaaagagaaatccctgggtgagctcatcaacatttgctccaacgatgggcagagaatgtttgaggcagcagccgttggcagcctgctggctggaggacccgttgttgccatcttaggcatgatttataatgtaattattctgggaccaacaggcttcctgggatcagctgtttttatcctcttttacccagcaatgatgtttgcatcacggctcacagcatatttcaggagaaaatgcgtggccgccacggatgaacgtgtccagaagatgaatgaagttcttacttacattaaatttatcaaaatgtatgcctgggtcaaagcattttctcagagtgttcaaaaaatccgcgaggaggagcgtcggatattggaaaaagctgggtacttccagagcatcactgtgggtgtggctcccattgtggtggtgattgccagcgtggtgaccttctctgttcatatgaccctgggcttcgatctgacagcagcacaggctttcacagtggtgacagtcttcaattccatgacttttgctttgaaagtaacaccgttttcagtaaagtccctctcagaagcctcagtggctgttgacagatttaagagtttgtttctaatggaagaggttcacatgataaagaacaaaccagccagtcctcacatcaagatagagatgaaaaatgccaccttggcatgggactcctcccactccagtatccagaactcgcccaagctgacccccaaaatgaaaaaagacaagagggcttccaggggcaagaaagagaaggtgaggcagctgcagcgcactgagcatcaggcggtgctggcagagcagaaaggccacctcctcctggacagtgacgagcggcccagtcccgaagaggaagaaggcaagcacatccacctgggccacctgcgcttacagaggacactgcacagcatcgatctggagatccaagagggtaaactggttggaatctgtggcagtgtgggaagtggaaaaacctctctcatttcagccattttaggccagatgacgcttctagagggcagcattgcaatcagtggaaccttcgcttatgtggcccagcaggcctggatcctcaatgctactctgagagacaacatcctgtttgggaaggaatatgatgaagaaagatacaactctgtgctgaacagctgctgcctgaggcctgacctggccattcttcccagcagcgacctgacggagattggagagcgaggagccaacctgagcggtgggcagcgccagaggatcagccttgcccgggccttgtatagtgacaggagcatctacatcctggacgaccccctcagtgccttagatgcccatgtgggcaaccacatcttcaatagtgctatccggaaacatctcaagtccaagacagttctgtttgttacccaccagttacagtacctggttgactgtgatgaagtgatcttcatgaaagagggctgtattacggaaagaggcacccatgaggaactgatgaatttaaatggtgactatgctaccatttttaataacctgttgctgggagagacaccgccagttgagatcaattcaaaaaaggaaaccagtggttcacagaagaagtcacaagacaagggtcctaaaacaggatcagtaaagaaggaaaaagcagtaaagccagaggaagggcagcttgtgcagctggaagagaaagggcagggttcagtgccctggtcagtatatggtgtctacatccaggctgctgggggccccttggcattcctggttattatggcccttttcatgctgaatgtaggcagcaccgccttcagcacctggtggttgagttactggatcaagcaaggaagcgggaacaccactgtgactcgagggaacgagacctcggtgagtgacagcatgaaggacaatcctcatatgcagtactatgccagcatctacgccctctccatggcagtcatgctgatcctgaaagccattcgaggagttgtctttgtcaagggcacgctgcgagcttcctcccggctgcatgacgagcttttccgaaggatccttcgaagccctatgaagttttttgacacgacccccacagggaggattctcaacaggttttccaaagacatggatgaagttgacgtgcggctgccgttccaggccgagatgttcatccagaacgttatcctggtgttcttctgtgtgggaatgatcgcaggagtcttcccgtggttccttgtggcagtggggccccttgtcatcctcttttcagtcctgcacattgtctccagggtcctgattcgggagctgaagcgtctggacaatatcacgcagtcacctttcctctcccacatcacgtccagcatacagggccttgccaccatccacgcctacaataaagggcaggagtttctgcacagataccaggagctgctggatgacaaccaagctcctttttttttgtttacgtgtgcgatgcggtggctggctgtgcggctggacctcatcagcatcgccctcatcaccaccacggggctgatgatcgttcttatgcacgggcagattcccccagcctatgcgggtctcgccatctcttatgctgtccagttaacggggctgttccagtttacggtcagactggcatctgagacagaagctcgattcacctcggtggagaggatcaatcactacattaagactctgtccttggaagcacctgccagaattaagaacaaggctccctcccctgactggccccaggagggagaggtgacctttgagaacgcagagatgaggtaccgagaaaacctccctctcgtcctaaagaaagtatccttcacgatcaaacctaaagagaagattggcattgtggggcggacaggatcagggaagtcctcgctggggatggccctcttccgtctggtggagttatctggaggctgcatcaagattgatggagtgagaatcagtgatattggccttgccgacctccgaagcaaactctctatcattcctcaagagccggtgctgttcagtggcactgtcagatcaaatttggaccccttcaaccagtacactgaagaccagatttgggatgccctggagaggacacacatgaaagaatgtattgctcagctacctctgaaacttgaatctgaagtgatggagaatggggataacttctcagtgggggaacggcagctcttgtgcatagctagagccctgctccgccactgtaagattctgattttagatgaagccacagctgccatggacacagagacagacttattgattcaagagaccatccgagaagcatttgcagactgtaccatgctgaccattgcccatcgcctgcacacggttctaggctccgataggattatggtgctggcccagggacaggtggtggagtttgacaccccatcggtccttctgtccaacgacagttcccgattctatgccatgtttgctgctgcagagaacaaggtcgctgtcaagggctgactcctccctgttgacgaagtctcttttctttagagcattgccattccctgcctggggcgggcccctcatcgcgtcctcctaccgaaaccttgcctttctcgattttatctttcgcacagcagttccggattggcttgtgtgtttcacttttagggagagtcatattttgattattgtatttattccatattcatgtaaacaaaatttagtttttgttcttaattgcactctaaaaggttcagggaaccgttattataattgtatcagaggcctataatgaagctttatacgtgtagctatatctatatataattctgtacatagcctatatttacagtgaaaatgtaagctgtttattttatattaaaataagcactgtgctaataacagtgcatattcctttctatcatttttgtacagtttgctgtactagagatctggttttgctattagactgtaggaagagtagcatttcattcttctctagctggtggtttcacggtgccaggttttctgggtgtccaaaggaagacgtgtggcaatagtgggccctccgacagccccctctgccgcctccccacggccgctccaggggtggctggagacgggtgggcggctggagaccatgcagagcgccgtgagttctcagggctcctgccttctgtcctggtgtcacttactgtttctgtcaggagagcagcggggcgaagcccaggccccttttcactccctccatcaagaatggggatcacagagacattcctccgagccggggagtttctttcctgccttcttctttttgctgttgtttctaaacaagaatcagtctatccacagagagtcccactgcctcaggttcctatggctggccactgcacagagctctccagctccaagacctgttggttccaagccctggagccaactgctgctttttgaggtggcactttttcatttgcctattcccacacctccacagttcagtggcagggctcaggatttcgtgggtctgttttcctttctcaccgcagtcgtcgcacagtctctctctctctctcccctcaaagtctgcaactttaagcagctcttgctaatcagtgtctcacactggcgtagaagtttttgtactgtaaagagacctacctcaggttgctggttgctgtgtggtttggtgtgttcccgcaaaccccctttgtgctgtggggctggtagctcaggtgggcgtggtcactgctgtcatcaattgaatggtcagcgttgcatgtcgtgaccaactagacattctgtcgccttagcatgtttgctgaacaccttgtggaagcaaaaatctgaaaatgtgaataaaattattttggattttgtaaaaaaaaaa

SEQ ID NO: 4 - DNA, 2554 bp, human
acaaggcagcctcgctcgagcgcaggccaatcggctttctagctagagggtttaactcctatttaaaaagaagaacctttgaattctaacggctgagctcttggaagacttgggtccttgggtcgcaggtgggagccgacgggtgggtagaccgtgggggatatctcagtggcggacgaggacggcggggacaaggggcggctggtcggagtggcggagcgtcaagtcccctgtcggttcctccgtccctgagtgtccttggcgctgccttgtgcccgcccagcgcctttgcatccgctcctgggcaccgaggcgccctgtaggatactgcttgttacttattacagctagagggtctcactccattgcccaggccagagtgcggggatatttgataagaaacttcagtgaaggccgggcgcggtggctcatgcccgtaatcccagcattttcggaggccgaggctggagtgcaatggtgtgatctcagctcactgcaacctctgcttcctgggtttaagtgattctcctgcctcagcctcccgagtagctgggattacaggcatcatggaccgatctaaagaaaactgcatttcaggacctgttaaggctacagctccagttggaggtccaaaacgtgttctcgtgactcagcaatttccttgtcagaatccattacctgtaaatagtggccaggctcagcgggtcttgtgtccttcaaattcttcccagcgcattcctttgcaagcacaaaagcttgtctccagtcacaagccggttcagaatcagaagcagaagcaattgcaggcaaccagtgtacctcatcctgtctccaggccactgaataacacccaaaagagcaagcagcccctgccatcggcacctgaaaataatcctgaggaggaactggcatcaaaacagaaaaatgaagaatcaaaaaagaggcagtgggctttggaagactttgaaattggtcgccctctgggtaaaggaaagtttggtaatgtttatttggcaagagaaaagcaaagcaagtttattctggctcttaaagtgttatttaaagctcagctggagaaagccggagtggagcatcagctcagaagagaagtagaaatacagtcccaccttcggcatcctaatattcttagactgtatggttatttccatgatgctaccagagtctacctaattctggaatatgcaccacttggaacagtttatagagaacttcagaaactttcaaagtttgatgagcagagaactgctacttatataacagaattggcaaatgccctgtcttactgtcattcgaagagagttattcatagagacattaagccagagaacttacttcttggatcagctggagagcttaaaattgcagattttgggtggtcagtacatgctccatcttccaggaggaccactctctgtggcaccctggactacctgccccctgaaatgattgaaggtcggatgcatgatgagaaggtggatctctggagccttggagttctttgctatgaatttttagttgggaagcctccttttgaggcaaacacataccaagagacctacaaaagaatatcacgggttgaattcacattccctgactttgtaacagagggagccagggacctcatttcaagactgttgaagcataatcccagccagaggccaatgctcagagaagtacttgaacacccctggatcacagcaaattcatcaaaaccatcaaattgccaaaacaaagaatcagctagcaaacagtcttaggaatcgtgcagggggagaaatccttgagccagggctgccatataacctgacaggaacatgctactgaagtttattttaccattgactgctgccctcaatctagaacgctacacaagaaatatttgttttactcagcaggtgtgccttaacctccctattcagaaagctccacatcaataaacatgacactctgaagtgaaagtagccacgagaattgtgctacttatactggttcataatctggaggcaaggttcgactgcagccgccccgtcagcctgtgctaggcatggtgtcttcacaggaggcaaatccagagcctggctgtggggaaagtgaccactctgccctgaccccgatcagttaaggagctgtgcaataaccttcctagtacctgagtgagtgtgtaacttattgggttggcgaagcctggtaaagctgttggaatgagtatgtgattctttttaagtatgaaaataaagatatatgtacagacttgtattttttctctggtggcattcctttaggaatgctgtgtgtctgtccggcaccccggtaggcctgattgggtttctagtcctccttaaccacttatctcccatatgagagtgtgaaaaataggaacacgtgctctacctccatttagggatttgcttgggatacagaagaggccatgtgtctcagagctgttaagggcttatttttttaaaacattggagtcatagcatgtgtgtaaactttaaatatgcaaataaataagtatctatgtctaaaaaaa

SEQ ID NO: 5 - DNA, 5518 bp, human
agggcgcgggagcgcggagcgagtttggttgcacttacaccggtacttaagcgcggaccggcgtgtccttggacttagagagtggggacgtccggcttcggagcgggagtgttcgttgtgccagcgactaaaaagagaattaaatatgggtgatgttgagaaaggcaagaagatttttattatgaagtgttcccagtgccacaccgttgaaaagggaggcaagcacaagactgggccaaatctccatggtctctttgggcggaagacaggtcaggcccctggatactcttacacagccgccaataagaacaaaggcatcatctggggagaggatacactgatggagtatttggagaatcccaagaagtacatccctggaacaaaaatgatctttgtcggcattaagaagaaggaagaaagggcagacttaatagcttatctcaaaaaagctactaatgagtaataattggccactgccttatttattacaaaacagaaatgtctcatgacttttttatgtgtaccatcctttaatagatctcatacaccagaattcagatcatgaatgactgacagaatattttgttgggcagtcctgatttaaaactaagactggcttgtggttaaatgaatatgttcagtttttgaattttaatagtaactccaattcagtaaatggtatcactgtttaccccttttaaagatatgattagacttcgttagtaatgttcaacttttcacaaagatggtgagtgccatcttaaaacttactggagattggttttatatttagatttatataactggttatgtgaatatatttaaatactggggaaattgcttcactgtcttagaaccaagcaagattcacctgtgttttgtgttcatgttcatttgcctcttaaaggcaagggttgaagataaataaggtagcaatgtctatagttttggccttaactatgccaatctaattataattccctgtatttaaaatggtttcttttacttattgaaaggcattttagtgtggtttatgtgtaatattaaagattattcaacacctctcacatcttacagatctataaggtcacatgcttttaaaatagtagcaagttaaacttcactcttgaattctttacaatctaagtcaaactaagttataatttaggattgtctttaaacagccattcagaaacaaaactgtagaactgtgtatttgattgggaatggtgcttttgccaacttaaaaggattaaagtaacggagatatacacaaattttaaaattatgtgtgatcacaagactaaagataattaaaaagaaaaccacagatcatgactttttgactgtgcttgatttcatgactgatgcacaaattttaatgattaaaaagtgcaggagccctaaatgtcagtgcagcagccctaaatgtcagtgcagcagtgttaaccagtcatggtgctagattgtttacttggttttctaggactgcctcaactagaataacacttcactaattgactcttagtttctttgctcagattgagaactgcagcatttatgccagacatggacagaggaatgcctgtggtcatagttttgtgatgtgtaacagtgtataattacatactgaattatttcatgcatagtctgtgccatacacatttagagtagtccttggagattttatggagatggtgagcacaaggtaagtcataaagaataatgagaaaataaatctatgctggtgcagctgagaactgtatctttgtgggacagtgagaagactgagaagatgtgaatccatggtctcaaaggtgatagggacgattagataggtgttttaaggcctgaaagcaatttataacatatgagtcttatttttatttatagaaatgtggaaagcttgctgtaattcatatttgaagtcctagtctgagttctggtggggaatttaaaaatgcatcctggaaatcctttaaagatttcagactttgaaaggccttgtagcagaggacttggtgactgtataaagttagtggtattcagggacagtgtagcaagtagcttacaaggggacaattctggactaatgagaaagacctgaagtgaaggctagagagttgattttttttttttttggcatcctggaaatgatacaggaaacatattaagatagatacagaaatgtgttcaaccttccatcttggctagttgtggcgtttagtttgttttttgagacatggtcacgctgtgtcgccaaggctggagtgcagtggtgcgatctcggctggctgcaacctctatttcccaggctcaagcgattctctcacttcagcttcccaagtagctgggactacaggtgttcgccaccatgcccagctaatttttttgtagagatggagttttgccatgtggtcccaggctggtctcaaactcctgagctcaagcaatccgtccacttgccttggctccccaaagtgctgggattacaggcgtgagccaccaggccctgcctggttttcaaattcagaaatcttattatttaacccagaagtaatcagcccagtagtaacttaggtttaattttttttcaggtttaaaatttttctcatttattttttctgagacggagtttcgcccttttcgcccaggctgagtacagtggtgcaatctcactgcaacctccgccttccagttgcaagtgattctcctgcctcagcctcctgagtagctgggattacaggcacccgccaccacgcctggctaatttttgtatttttagtggagatggtgtttcaccatgttggccagactggtcttggactcctgacctcgtgatccacccaccttggcctcccaaagttctaggattacaggtgtgagccaccacgtccggccaatttttctcatttctatgcctcctatattaaggtctgtgttggcacagatgagtaactgccatgttctaggtcagttatacccaagcacttctggtggtttaaaatgtgattctgtaacttttttatttttatttttttgagataatttcactcttgttgcccaggctggagtgcaatggcgtgatcgctgctcaccgcaacctccgccttccaggttcaagcgattctcctgactcagcctctcaagtagctgggattacaagcatgcgccaccatgcccagcttattttgtgtttttagtagagacagggtttctccatgctggacaggctggtcttgaactcccgacgtcgggtgatctgcctgcctcggcctcccaaagtgctgggattacaggcgtgagccaccacgcctggccaattatgtaattttttaaaaaggacatttctatcagggatatataccttcagaaataaggaaataggggaaaaaaagagcactataaaccacatgttttcatttctagtgcttcgctgtaagtggctaggttggtagaatcaaaaacaagggccagatgtatttaaggggtattcagatgccacctacatgcttattttgtctagaacagtgctgtctaatagaactttctgtgacgatggatattttgtagacttttgctgtccagtgtggtagccactaaccacatgtggctgttaagcccttgaaatatagctagtgtgactagaaagtattttattttaaatttacataggcacaagtggctagtggctactgtattgacattctgggtctaggactagaaccagtggtctgtaacaaaagtactttctctttactctattaaatctagaattagccgggcatggtcgctcatgcctgtaatcccagcactttgggaggccaaggcaggcagatcacttgaggtcaggcgtttgagaccagcctggtcaacatggcgaaaccctgtctctacaaaaaacataaaagttagccaggtgtggtggtgggcacctgtaatctcagctacttgggaggctgaggcacaagaatcacttgaacttgggaggtggaggttgcagtgagccaagattgtgccactgcactcaagcctgggtgacgagtgaaactgtctccaaaaaaaaaaaaaaatctagaattcttggaagtacattatattgcccttcagaatagattccagttcctgttgtgctcacctttataattttaccataagttttacctattcgtaagttggcagttttagatagataacattctggtggtagctagggatttaccttttgtcatccttttcctgcacttctcttgaattcctttatagatgtacagttttgctttaaccactgaagattgctgtaaattataaagggtgtgatagaatccacatggctgtcaagaaggagatcttaccaaggacagttgactgactagtctcagattgtttcatatcatttatacttgggtaagagtaaactagataactgggcgtcgtggtgcacacctgtagtcccagccactctgaggcaggaggactgcttgtgcccagaagttcgaggctgcagtgtagctgtgattgtgctactgcactccagcctgggcaacagataaaggaaactccatctcttttaaaaaaaaaaaaagtggtctgggtgcagtaggtcatgcctgcaatcccagcactttgggaggccaaggcaggcagatcacctgaggtcaggagcttgagaccagcctggccaacatggtgaaaccccatctctaccaaaaatataaaaactagctgggcgtggtggcggcacctgtaattgcagctatttgagaggctgaggcaggagaatcgcttgaacctgggagatgggggttgcagtgagccaagaccgccccatcgcactccagcctgggcaacaatagtgaaactccgtctcaaaaagaaaaaaagtttccttagaatggaaaatattcattcatgagctcttttggcaatccgtcatcagtatattctgaaaaccaataagatgttgccaagttgggggcgagagctatgtaatgcaaggcatatgcctgatgaagtatacaaatacacctgaccagaaactttgtctcccacataagtctcttctaggcactgtcggggtacatactgagctgctgctttggctgtattttctgtgcctcagaataaccattgtcctggtgttcatatccttagagttcagtacaaaatgttggatatccatttaataggttccaggttatcttagttggagtttggggtatttgaaaacgtcatgccttcaggctatcatttccctcagaaagctaagtaaatttactgcattcatttctcaaagagtaaaagtgcaggttgtatgtgtctatgaacatttaaacatgttaaaatgttaaatttaacattttaaatttaaacatttaaatatgtctgtaacttgaacagtgtagtttcagaaaggaccactgggctagtgtaatgcagaaaatgctgggtctaggattaggagaaaattgtgtttagtgtgtatcaataaacagcccgtggacccaaaaaaaaaaaa

SEQ ID NO: 6 - DNA, 3931 bp, human
gagagtgagagaaaggctggaagacgagacctgctcactctgtcaccgaggctagagtacagtggcacaatcacagctcattgcagcctcaacctcccaggctcaatcgatccttccaactgagcctccctagcagctgggactattagtgcacaccaccacacctagcctgcaggatgtttcctcaatgaggggaaggctgctgcacaatggcagtttttgatactcctgaggaggcctttggtgtcttacgtccagtctgtgttcagctcacaaagacccagacagtggagaatgtggagcatctgcagacacgactacaagctgtgagtgacagtgcccttcaggaacttcagcagtacatcctcttccctctgcgatttaccctgaagaccccaggtcccaaaagagagcgtttgatccaaagtgtggtggaatgcctcacatttgtcctttcttcaacatgtgtgaaagaacaggagcttctccaggaactcttttcagaactctctgcttgtctgtattcacccagctcccaaaaacctgcggctgtgtccgaggagttgaaattggctgtgatccagggacttagcacattaatgcactcagcttatggggacatcattctgactttttatgagccctccattctgccacgtttaggatttgctgtatctttactgttaggccttgcagaacaggagaaatcaaagcaaattaaaattgctgccttaaaatgtttacaggttctactcttgcagtgtgattgtcaggaccatccaaggtcattggatgaacttgaacaaaagcagctgggggatttgtttgcctcttttttacctggaatctcaactgcactgaccaggcttatcacaggagactttaaacaaggtcacagcattgtcgtatcttccctaaagatcttttacaagacagtgagcttcattatggctgatgaacagctcaaaagaatctcaaaggtccaagcaaaacctgcagttgagcacagagtagcagagctgatggtttacagggaagcagattgggtaaaaaagactggcgacaagttgactatccttattaaaaagataattgagtgtgtttctgttcacccacactggaaggtgagactggaactggtagaacttgtggaggaccttcttttgaagtgcagtcaatcattggtcgaatgtgctggtccccttctgaaggccttagtgggactagtaaatgatgagagtcctgaaatccaagcccagtgcaataaagttctgagacattttgcagatcaaaaagtagtggtgggcaacaaagccctcgctgacatcttgtcagaaagcctgcattcccttgccacatctcttcctcgcctaatgaactcccaagatgaccagggcaaattctctactctttccttgttacttggttatctgaaactcttgggcccaaaaataaactttgtcctcaactctgtggcccatctccagcggctttccaaagcactcatccaagttctagagctagacgtggctgacatcaagattgttgaggaacggcgttggaactctgatgatctgaatgcttctccaaagacctcagccacacagccttggaaccgcatccagaggagatatttccgcttcttcactgatgagagaatcttcatgctcttgaggcaggtttgtcagctacttggttattatgggaatctttatttgcttgtggatcactttatggaactttaccatcaatctgtggtttaccggaagcaagctgccatgatccttaatgaactggttacaggggctgctgggctggaggttgaggatcttcacgaaaaacatattaaaacaaacccagaagaactgagagagattgtgacatctatacttgaagaatacacaagtcaagaaaattggtatttggttacctgtcttgaaactgaggaaatgggagaggagctgatgatggagcacccaggcctccaagccatcacgtctggtgaacacacctgccaagttacatcttttctagccttctcaaagccaagtcccactatttgctccatgaacagtaacatctggcaaatatgcattcagttggaaggaattggccagtttgcatatgcactaggaaaagacttctgtttgctcttgatgtcagccctttatccagtactggagaaggctggagaccaaaccctactcattagtcaggtggctaccagcaccatgatggacgtttgccgtgcttgtggctacgactccctgcagcacctgatcaatcaaaattcagactatttagtgaatgggatctctttaaatctgcgtcatctggctctgcatcctcataccccaaaggtcctggaagtcatgctgcggaactcagatgctaacctgcttcctttggtggcagatgtggttcaagatgtcttggccaccctggaccaattttacgataagagagctgcttcctttgtcagcgttctgcatgctctgatggcagcattagcccagtggttcccagacacaggtaatcttgggcacctccaagagcaaagtttaggagaagagggaagtcatttgaaccaaagaccagcagctcttgagaagagcaccaccacagctgaagacatcgaacagtttttgctgaactacctcaaagagaaggatgtggcagatggaaatgtctcggattttgataatgaagaagaggaacagtcagtccctcccaaagtggatgagaatgacacccgtccagatgtggagccaccactgccattgcagatccaaatagccatggacgtgatggaacgctgcatccacttgttgtcagataaaaatctgcaaatccgcctgaaggtcttggatgtgctggatctgtgtgtggttgttcttcagtcccacaaaaaccagctgcttcccttggctcatcaggcctggccctcgctcgttcaccgactcacacgggacgcccccctggcagtgcttagagccttcaaggttttacgtaccctgggaagcaagtgtggtgactttcttcgcagccggttctgcaaagatgtcctgccaaagctggctggctccctagtcacccaggcccccatcagtgccagggctggaccagtttactcgcacacgctggccttcaagttgcagctggctgtcttacagggcctgggccccctctgtgagagactggacctaggtgagggtgacctgaataaagtggctgatgcctgcttgatttacctcagtgtcaaacagcccgtgaaattacaagaggctgccaggagcgtcttcctccacttgatgaaggtggacccagactccacctggttcctcctgaacgagctttactgccccgtgcagttcacacctccccaccccagcctccaccctgtgcagctgcacggggccagcgggcagcagaacccctacacgaccaacgtgctccagctgctcaaggagctgcagtgaccctgctcccccaccacagaggccaccgatccctcccctactgccagccagaagctgggctgaccccaccccggccataggcggtggcagcggcagcagagaaggtgaattagttagccaatcgatttataaattgatcgatcacacaactgcttagaaatggattgaaggaaagtagctgactattatttatatttcataccttgtgttttcaagtgacattgtctggtggctctaagggtttaaccccttagcctaccatctctatagccccagctccctcacaggccacacacacacacacacaagaggtcagttcccctccatctgcatacacctccctgtcttcaaataatgagatggaactaatttgttttacctaacctgatctttgggaaacaaacggaaataaagacacttcttggatgaaaagtaaaaaaaaaaaaaaaaaa

SEQ ID NO: 7 - DNA, 1833 bp, human
tgcgcgccgcccggccaggcccgcaaagaggcctccgagcgccatggctgcgcccccggcccgcgcggacgctgatccttcgcccacgtcgccacctacggcccgagacacaccaggccggcaggctgagaaaagcgagaccgcgtgcgaggaccgcagcaatgcagagtccctggacaggctcctgccacctgtgggcactgggcgctctccccggaagcggaccaccagccagtgcaagtcagagcctcccctgctgcgtacaagcaagcgtaccatctacaccgccgggcggccgccctggtacaatgaacacggcacgcaatccaaagaggccttcgccatcggcttgggaggcggcagtgcctctgggaagaccactgtggccagaatgatcatcgaggccctggatgtgccctgggtggtcttgctgtccatggactccttctacaaggtgctgactgagcagcagcaggaacaggccgcacacaacaacttcaacttcgaccacccagatgcctttgacttcgacctcatcatttccaccctcaagaagctgaagcaggggaagagtgtcaaggtgcccatttatgacttcaccacgcacagccggaagaaggactggaaaacactgtatggtgcaaacgtcatcatctttgagggcatcatggcctttgctgacaagacactgttggagctcctggacatgaagatctttgtggacacagactccgacatccgcctggtacggcggctgcgccgggacatcagtgagcgcggccgggacatcgagggtgtcatcaagcagtacaacaagtttgtcaagccctccttcgaccagtacatccagcccaccatgcgcctggcagacatcgtggtccccagagggagcggcaacacggtggccatcgacctgattgtgcagcacgtgcacagccagctggaggagcgtgaactcagcgtcagggctgcgctggcctcggcacaccagtgccacccgctgccccggacgctgagcgtcctgaagagcacgccgcaggtacggggcatgcacaccatcatcagggacaaggagaccagtcgcgacgagttcatcttctactccaagagactgatgcggctgctcatcgagcacgcgctctccttcctgccctttcaggactgcgtcgtacagaccccgcaggggcaggactatgcgggcaagtgctatgcggggaagcagatcaccggtgtgtccattctgcgcgccggtgaaaccatggagcccgcgctgcgcgctgtgtgcaaagacgtgcgcatcggcaccatcctcatccagaccaaccagcttaccggggagcccgagctccactacctgaggctgcccaaggacatcagcgatgaccacgtgatcctcatggactgcaccgtgtccacgggcgcggcggccatgatggcagtgcgcgtgctcctggaccacgacgtgcctgaggacaagatctttttgctgtcgctgctcatggcagagatgggcgtgcactcagtggcctatgcatttccgcgagtgagaatcatcaccacggcggtggacaagcgggtcaatgaccttttccgcatcatcccaggcattgggaactttggcgaccgctactttgggacagacgcggtccccgatggcagtgacgaggaggaagtggcctacacgggttagctgcccagtgagccatcccgtccccaccaccctcctcctgcctcctgacccaggactgctgaatacaaagatgttaatttttaaaatgttactagtataatttattctatgcattttataaaataaataaagctttagaaaaa

SEQ ID NO: 8 - DNA, 2874 bp, human
gcgtctggagaaaaccctagcgggtggctccgtgcggccagagctctagagagtggtgccgccttccaaccttcttccccaagccctggtggccggctccgcctcttctcgaatcttttccacagcccaaaatggccgcagaggtgtattttggcgatctagagctcttcgagccgttcgaccacccagaggagtcgattccgaagcccgttcacactcgcttcaaggacgacgacggcgacgaggaggacgaaaatggggtcggcgacgcggagctacgggagcggcttcggcagtgcgaggagaccatcgagcagctccgcgccgagaatcaagaacttaaacgaaaattgaacattctgactcgaccgagtggaatattggtgaacgatactaagttagatggacctatattacagattctattcatgaacaatgctatttcaaagcaatatcatcaagaaatagaggaatttgtatcaaatttagtaaaaagatttgaggaacagcagaaaaatgatgtggaaaagacttcctttaatcttttgccccagccatccagtattgtgctagaggaggaccacaaagtggaagagtcctgtgccattaaaaacaacaaggaagctttcagtgttgtaggaagtgtcctgtattttactaatttttgccttgataaattggggcaaccgcttctaaatgaaaaccctcagctttccgaaggatgggaaatacccaagtaccatcaagtcttcagccacattgtttctctagaagggcaagaaatacaagtaaaggcaaaaaggccaaagcctcactgtttcaattgtggttctgaagaacaccaaatgaaagattgcccaatgcctcggaatgctgctcgaataagtgaaaagagaaaagagtatatggatgcctgtggagaagcaaacaatcagaatttccagcagcgataccacgcagaagaagtagaagaaagatttggaagattcaagccaggagttattagtgaggaacttcaagatgcactaggtgtgacagacaagagtcttccaccttttatctatcggatgcgccagctagggtacccaccagggtggctcaaagaggctgaattggagaattcggggcttgcactctatgatggaaaagatggcactgatggggaaacagaagttggagaaatacaacagaataaaagtgtcacttacgatctctcaaaattggtcaactatcctggttttaatatatctactcccagaggaattccagacgaatggaggatctttggttccataccaatgcaggcatgtcagcagaaggatgtgtttgccaattaccttacttctaacttccaagcgccaggtgtgaagtctggcaacaagaggtcttcatctcactctagcccaggtagtccaaagaagcagaagaatgaaagcaactcagcgggatctcccgccgacatggagctcgattcagatatggaggtaccacatggttctcagagcagcgaaagttttcagtttcaaccaccattacctcctgacactcctccactcccccggggaactcctccacccgtcttcacccctccactcccaaagggcaccccgccgctgactcccagtgactcaccccagaccagaacagcatctggagctgtggatgaggacgcactgactctagaagaacttgaagaacagcagaggcggatctgggcagctcttgagcaggccgagagcgtaaacagcgactccgacgttcctgtggacacacctttaactggcaattccgttgcctcatcaccttgtccaaatgagctagacctccctgtcccggagggaaaaacatctgaaaagcagacgctggatgagcctgaggtaccagagatttttacaaagaaatcagaagctggacatgcctccagtccagactctgaggtgacatcactttgtcagaaggaaaaagcagagttggctccggtaaacactgaaggtgcccttcttgataatggcagtgtcgtaccaaactgtgacatcagcaatgggggcagccagaagctctttcctgcagacaccagtccttcaacggccactaaaattcatagccctatacctgacatgagcaaatttgcaactggaatcacgccatttgaatttgagaatatggcagaatctactggaatgtacctcaggataagaagcttgttaaagaactcaccccgaaaccagcagaaaaacaaaaaggcctctgaataatggcttgacttagcactgagagctatttaataactttgttccgttaattagtactaattaagtggatagatagaatggttttcctgtttgtccctcccatgtttaaaaatctatccaaggttcatgttccaaagtcaagcctattttaaagaaagactgagctcactagttcagtatattttattctcactgacaaaacttggggggagatgtgaatatgacctggtttagagagggtttgttaaggtttatactatttttggattgtgactatccgtcgagagtgatggtttttatctgtcttttgtacattgttttccctttctacattttgctaattatcctgtatataagtttaatatatcactttttaaaagaaaaaattctaccattttaaattcatgtttcaactcctacaaccaaatgagaaaaatcagggatgagcagctttatcccatttggggtatttttgtaagtgatttacatgtgtcaattttagtaatacttttacttttttgtaacttcatccttcatatatgcttgctatacaggtatgttcatctttgtgtacagaggtttaataaattagttttcatatacataatctaaaaaaaaaaaa

SEQ ID NO: 9 - DNA, 3237 bp, human
gcgaagtgaagggtggcccaggtggggccaggctgactgaatgtatctcctagctatggactaaataatacatggggggaaataaacaagtattcatgagggtgaaaatgtgacccagcaggaaaattacaactattttcaattgacgttgaataggatgagtcatggaatttaagtgatttactgaagattatactactggtagatagaagagctaaagaaagatggatactatgatgctgaatgtgcggaatctgtttgagcagcttgtgcgccgggtggagattctcagtgaaggaaatgaagtccaatttatccagttggcgaaggactttgaggatttccgtaaaaagtggcagaggactgaccatgagctggggaaatacaaggatcttttgatgaaagcagagactgagcgaagtgctctggatgttaagctgaagcatgcacgtaatcaggtggatgtagagatcaaacggagacagagagctgaggctgactgcgaaaagctggaacgacagattcagctgattcgagagatgctcatgtgtgacacatctggcagcattcaactaagcgaggagcaaaaatcagctctggcttttctcaacagaggccaaccatccagcagcaatgctgggaacaaaagactatcaaccattgatgaatctggttccattttatcagatatcagctttgacaagactgatgaatcactggattgggactcttctttggtgaagactttcaaactgaagaagagagaaaagaggcgctctactagccgacagtttgttgatggtccccctggacctgtaaagaaaactcgttccattggctctgcagtagaccaggggaatgaatccatagttgcaaaaactacagtgactgttcccaatgatggcgggcccatcgaagctgtgtccactattgagactgtgccatattggaccaggagccgaaggaaaacaggtactttacaaccttggaacagtgactccaccctgaacagcaggcagctggagccaagaactgagacagacagtgtgggcacgccacagagtaatggagggatgcgcctgcatgactttgtttctaagacggttattaaacctgaatcctgtgttccatgtggaaagcggataaaatttggcaaattatctctgaagtgtcgagactgtcgtgtggtctctcatccagaatgtcgggaccgctgtccccttccctgcattcctaccctgataggaacacctgtcaagattggagagggaatgctggcagactttgtgtcccagacttctccaatgatcccctccattgttgtgcattgtgtaaatgagattgagcaaagaggtctgactgagacaggcctgtataggatctctggctgtgaccgcacagtaaaagagctgaaagagaaattcctcagagtgaaaactgtacccctcctcagcaaagtggatgatatccatgctatctgtagccttctaaaagactttcttcgaaacctcaaagaacctcttctgacctttcgccttaacagagcctttatggaagcagcagaaatcacagatgaagacaacagcatagctgccatgtaccaagctgttggtgaactgccccaggccaacagggacacattagctttcctcatgattcacttgcagagagtggctcagagtccacatactaaaatggatgttgccaatctggctaaagtctttggccctacaatagtggcccatgctgtgcccaatccagacccagtgacaatgttacaggacatcaagcgtcaacccaaggtggttgagcgcctgctttccttgcctctggagtattggagtcagttcatgatggtggagcaagagaacattgaccccctacatgtcattgaaaactcaaatgccttttcaacaccacagacaccagatattaaagtgagtttactgggacctgtgaccactcctgaacatcagcttctcaagactccttcatctagttccctgtcacagagagtccgttccaccctcaccaagaacactcctagatttgggagcaaaagcaagtctgccactaacctaggacgacaaggcaacttttttgcttctccaatgctcaagtgaagtcacatctgcctgttacttcccagcattgactgactataagaaaggacacatctgtactctgctctgcagcctcctgtactcattactacttttagcattctccaggcttttactcaagtttaattgtgcatgagggttttattaaaactatatatatctccccttccttctcctcaagtcacataatatcagcactttgtgctggtcattgttgggagcttttagatgagacatctttccaggggtagaagggttagtatggaattggttgtgattctttttggggaagggggttattgttcctttggcttaaagccaaatgctgctcatagaatgatctttctctagtttcatttagaactgatttccgtgagacaatgacagaaaccctacctatctgataagattagcttgtctcagggtgggaagtgggagggcagggcaaagaaaggattagaccagaggatttaggatgcctccttctaagaaccagaagttctcattccccattatgaactgagctataatatggagctttcataaaaatgggatgcattgaggacagaactagtgatgggagtatgcgtagctttgatttggatgattaggtctttaatagtgttgagtggcacaaccttgtaaatgtgaaagtacaactcgtatttatctctgatgtgccgctggctgaactttgggttcatttggggtcaaagccagtttttcttttaaaattgaattcattctgatgcttggcccccatacccccaaccttgtccagtggagcccaacttctaaaggtcaatatatcatcctttggcatcccaactaacaataaagagtaggctataagggaagattgtcaatattttgtggtaagaaaagctacagtcattttttctttgcactttggatgctgaaatttttcccatggaacatagccacatctagatagatgtgagctttttcttctgttaaaattattcttaatgtctgtaaaaacgattttcttctgtagaatgtttgacttcgtattgacccttatctgtaaaacacctatttgggataatatttggaaaaaaagtaaatagctttttcaaaatgaaaaaaaaaa

SEQ ID NO: 10 - DNA, 3324 bp, human
acttcctgccgggctgcgggcgcctgagcggctcttcagcgtttgcgccggcggctgccgcgtctctctcggctcccgcttcctttgaccgcctcccccccccggcccggcggcgcccgcctcctccacggccactccgcctcttccctcccttcgtcccttcttcctctcccttttttccttcttccttcccctcctcgccgccaccgcccaggaccgccggccgggggacgagctcggagcagcagccagaaagcataacccctactgtagaactaaatgcactgtgcatgaaacttggaaaaaaaccaatgtataagcctgttgacccttactctcggatgcagtccacctataactacaacatgagaggaggtgcttatcccccgaggtacttttacccatttccagttccacctttactttatcaagtggaactttctgtgggaggacagcaatttaatggcaaaggaaagacaagacaggctgcgaaacacgatgctgctgccaaagcgttgaggatcctgcagaatgagcccctgccagagaggctggaggtgaatggaagagaatccgaagaagaaaatctcaataaatctgaaataagtcaagtgtttgagattgcacttaaacggaacttgcctgtgaatttcgaggtggcccgggagagtggcccaccccacatgaagaactttgtgaccaaggtttcggttggggagtttgtgggggaaggtgaagggaaaagcaagaagatttcaaagaaaaatgccgccatagctgttcttgaggagctgaagaagttaccgcccctgcctgcagttgaacgagtaaagcctagaatcaaaaagaaaacaaaacccatagtcaagccacagacaagcccagaatatggccaggggatcaatccgattagccgactggcccagatccagcaggcaaaaaaggagaaggagccagagtacacgctcctcacagagcgaggcctcccgcgccgcagggagtttgtgatgcaggtgaaggttggaaaccacactgcagaaggaacgggcaccaacaagaaggtggccaagcgcaatgcagccgagaacatgctggagatccttggtttcaaagtcccgcaggcgcagcccaccaaacccgcactcaagtcagaggagaagacacccataaagaaaccaggggatggaagaaaagtaaccttttttgaacctggctctggggatgaaaatgggactagtaataaagaggatgagttcaggatgccttatctaagtcatcagcagctgcctgctggaattcttcccatggtgcccgaggtcgcccaggctgtaggagttagtcaaggacatcacaccaaagattttaccagggcagctccgaatcctgccaaggccacggtaactgccatgatagcccgagagttgttgtatgggggcacctcgcccacagccgagaccattttaaagaataacatctcttcaggccacgtaccccatggacctctcacgagaccctctgagcaactggactatctttccagagtccagggattccaggttgaatacaaagacttccccaaaaacaacaagaacgaatttgtatctcttatcaattgctcctctcagccacctctgatcagccatggtatcggcaaggatgtggagtcctgccatgatatggctgcgctgaacatcttaaagttgctgtctgagttggaccaacaaagtacagagatgccaagaacaggaaacggaccaatgtctgtgtgtgggaggtgctgaaccttttctggccatgaaccattataaaatcccaacatatatactgaaaatactgaaactgctttgaaaatttggaatttctgatacctccagtgggccgagagacacggtgggtaaaggatgtgggcagcagcagggaagacaacagaaacacaaggaggcggctgtggccgggctggactgtgctggggtttgttgtgatggccactcggtgacctggcggtccctacgcaatagcagctgcctgtggggaagaagggctgcccagccagctggttctcccgggacaccagcagatccacaccctgggcacctccgtgtttggtcttttttttcccctgtgtgaaagaagaaacggcacgaccccttctcaagctggctcactcagacacattgggacaaaccctggacagccatgccagagagaggcctttgaccggccccagagctaaaagcaccagagaaaatcaaatgcttcctactcagcgtgacccaacttttctagtgtgccacggccccaccacctcctgcagtacccacaccatcaccactgctttctcttccaacagtgatctgtattcttagtttcattattttcttttgattgatatgacactatataaaattttcatttgagaatttctcaattgtatctagttaaatagcacagtttggaaacttgtctgagactgactttatcaataatctaaccgacaaagatcatatccatgtgtatgtggttagacatttttatttcattgactaacccaggacagtttcagtgatgcaaattgtgtgccctctggttcagctgaaacagtcctggactttcaaaaaccttgaataagtctcccacagttgtataaattggacaatttaggaattttaaactttagatgatcatttggttccatttttatttcatttttatttttgttaatgcaaacaggacttaaatgaactttgatctctgttttaaagattattaaaaaacattgtgtatctatacatatggctcttgaggacttagctttcactacactacaggatatgatctccatgtagtccatataaacctgcagagtgattttccagagtgctcgatactgttaattacatctccattagggctgaaaagaatgacctacgtttctgtatacagctgtgttgcttttgatgttgtgttactgtacacagaagtgtgtgcactgaggctctgcgtgtggtccgtatggaaagcctggtagccctgcgagttaagtactgcttccattcattgtttacgctggaatttttctccccatggaatgtaagtaaaacttaagtgtttgtcatcaataaatggtaatactaaatttttttgttaatttattctcaaatgccactactgctaggttggtcccctcccaacttgcccctatcctttttttatttttaagaaaaaaactttgtaatgcttgtgattccatctgggcaaaagtctgaagatctgaaataactttatatcaaaccattgatcaataaacagctactaatgaaaaaa

SEQ ID NO: 11 - DNA, 1388 bp, human
gtgcgtgagtttggtggcggccggctgtgcagagacgccatgtaccggctcatgtcagcagtgactgcccgggctgccgcccccgggggcttggcctcaagctgcggacgacgcggggtccatcagcgcgccgggctgccgcctctcggccacggctgggtcgggggcctcgggctggggctggggctggcgctcggggtgaagctggcaggtgggctgaggggcgcggccccggcgcagtcccccgcggcccccgaccctgaggcgtcgcctctggccgagccgccacaggagcagtccctcgccccgtggtctccgcagaccccggcgccgccctgctccaggtgcttcgccagagccatcgagagcagccgcgacctgctgcacaggatcaaggatgaggtgggcgcaccgggcatagtggttggagtttctgtagatggaaaagaagtctggtcagaaggtttaggttatgctgatgttgagaaccgtgtaccatgtaaaccagagacagttatgcgaattgctagcatcagcaaaagtctcaccatggttgctcttgccaaattgtgggaagcagggaaactggatcttgatattccagtacaacattatgttcccgaattcccagaaaaagaatatgaaggtgaaaaggtttctgtcacaacaagattactgatttcccatttaagtggaattcgtcattatgaaaaggacataaaaaaggtgaaagaagagaaagcttataaagccttgaagatgatgaaagagaatgttgcatttgagcaagaaaaagaaggcaaaagtaatgaaaagaatgattttactaaatttaaaacagagcaggagaatgaagccaaatgccggaattcaaaacctggcaagaaaaagaatgattttgaacaaggcgaattatatttgagagaaaagtttgaaaattcaattgaatccctaagattatttaaaaatgatcctttgttcttcaaacctggtagtcagtttttgtattcaacttttggctataccctactggcagccatagtagagagagcttcaggatgtaaatatttggactatatgcagaaaatattccatgacttggatatgctgacgactgtgcaggaagaaaacgagccagtgatttacaatagagcaaggtaaatgaataccttctgctgtgtctagctatatcgcatcttaacactattttattaattaaaagtcaaattttctttgtttccattccaaaatcaacctgccacattttgggagcttttctacatgtctgttttctcatctgtaaagtgaaggaagtaaaacatgtttataaagtaaaaaaaaaaaaaaaaaaaaaaaaaaaaaaaaaaaaaaaaaaaaaaaaaaaaaaa

SEQ ID NO: 12 - DNA, 1841 bp, human
actgcgccgccaccgtcaataggtggaccccctcccggagataaaaccgccggcgccggcgccgccagtccctctggctgagacctcggctccggaatcactgcagcccccctcgccctgagccagagcaccccgggtcccgccagcccctcacactcccagcaaaatgggcaaggagaagacccacatcaacatcgtggtcatcggccacgtggactccggaaagtccaccaccacgggccacctcatctacaaatgcggaggtattgacaaaaggaccattgagaagttcgagaaggaggcggctgagatggggaagggatccttcaagtatgcctgggtgctggacaagctgaaggcggagcgtgagcgcggcatcaccatcgacatctccctctggaagttcgagaccaccaagtactacatcaccatcatcgatgcccccggccaccgcgacttcatcaagaacatgatcacgggtacatcccaggcggactgcgcagtgctgatcgtggcggcgggcgtgggcgagttcgaggcgggcatctccaagaatgggcagacgcgggagcatgccctgctggcctacacgctgggtgtgaagcagctcatcgtgggcgtgaacaaaatggactccacagagccggcctacagcgagaagcgctacgacgagatcgtcaaggaagtcagcgcctacatcaagaagatcggctacaacccggccaccgtgccctttgtgcccatctccggctggcacggtgacaacatgctggagccctcccccaacatgccgtggttcaagggctggaaggtggagcgtaaggagggcaacgcaagcggcgtgtccctgctggaggccctggacaccatcctgccccccacgcgccccacggacaagcccctgcgcctgccgctgcaggacgtgtacaagattggcggcattggcacggtgcccgtgggccgggtggagaccggcatcctgcggccgggcatggtggtgacctttgcgccagtgaacatcaccactgaggtgaagtcagtggagatgcaccacgaggctctgagcgaagctctgcccggcgacaacgtcggcttcaatgtgaagaacgtgtcggtgaaggacatccggcggggcaacgtgtgtggggacagcaagtctgacccgccgcaggaggctgctcagttcacctcccaggtcatcatcctgaaccacccggggcagattagcgccggctactccccggtcatcgactgccacacagcccacatcgcctgcaagtttgcggagctgaaggagaagattgaccggcgctctggcaagaagctggaggacaaccccaagtccctgaagtctggagacgcggccatcgtggagatggtgccgggaaagcccatgtgtgtggagagcttctcccagtacccgcctctcggccgcttcgccgtgcgcgacatgaggcagacggtggccgtaggcgtcatcaagaacgtggagaagaagagcggcggcgccggcaaggtcaccaagtcggcgcagaaggcgcagaaggcgggcaagtgaagcgcgggcgcccgcggcgcgaccctccccggcggcgccgcgctccgaaccccggcccggcccccgccccgcccccgccccgcgcgccgctccggcgccccgcacccccgccaggcgcatgtctgcacctccgcttgccagaggccctcggtcagcgactggatgctcgccatcaaggtccagtggaagttcttcaagaggaaaggcgcccccgccccaggcttccgcgcccagcgctcgccacgctcagtgcccgttttaccaataaactgagcgaccccag

SEQ ID NO: 13 - DNA, 1815 bp, human
agcagtcgcttagtaacttgcagcggctacatcaactttggaccaattcgcaaactgctggtgtaaagcgtttttgcgcatgtgctgtcgccttgcgggaaaaggagcctttttcttcgacgatttccgggcgacgcaggaagtggctccagggcgcacgcgcgttgtttccgcggtagtcagggcagtttctaccgcaggcttaaggaggcttcgggctcctgggatttctgtccgcgctcctggccctcgtccttcgcgccagagcaggttcgcaaactcctcagacccttctgctcccggccgccgctttccgccggggcgagacccccaggttcaaaatgagcctgtttggaacaacctcaggttttggaaccagtgggaccagcatgtttggcagtgcaactacagacaatcacaatcccatgaaggatattgaagtaacatcatctcctgatgatagcattggttgtctgtcttttagcccaccaaccttgccggggaactttcttattgcaggatcatgggctaatgatgttcgctgctgggaagttcaagacagtggacagaccattccaaaagcccagcagatgcacactgggcctgtgcttgatgtctgctggagtgacgatgggagcaaagtgtttacggcatcgtgtgataaaactgccaaaatgtgggacctcagcagtaaccaagcgatacagatcgcacagcatgatgctcctgttaaaaccatccattggatcaaagctccaaactacagctgtgtgatgactgggagctgggataagactttaaagttttgggatactcgatcgtcaaatcctatgatggttttgcaactccctgaaaggtgttactgtgctgacgtgatataccccatggctgtggtggcaactgcagagaggggcctgattgtctatcagctagagaatcaaccttctgaattcaggaggatagaatctccactgaaacatcagcatcggtgtgtggctatttttaaagacaaacagaacaagcctactggttttgccctgggaagtatcgaggggagagttgctattcactatatcaaccccccgaaccccgccaaagataacttcacctttaaatgtcatcgatctaatggaaccaacacttcagctcctcaggacatttatgcggtaaatggaatcgcgttccatcctgttcatggcacccttgcaactgtgggatctgatggtagattcagcttctgggacaaagatgccagaacaaaactaaaaacttcggaacagttagatcagcccatctcagcttgctgtttcaatcacaatggaaacatatttgcatacgcttccagctacgactggtcaaagggacatgaattttataatccccagaaaaaaaattacattttcctgcgtaatgcagccgaagagctaaagcccaggaataagaagtagtggctggagactctggctcagccagagttgtttctctccactctgcctcatctctgtacgaatttgggtcccagccttgttgggttgtcagccatggacatggatttcaacccctggagaaaacgatgtcattgttcagcagctgagagcccaggcgtccgcggcgacttgccgtctctccattccactgcctgttgcagagtttttctgtaactaagggggttgaggttattgtagacgttagattgcgggcaccgccagggattttgcagcgcttcagtgtacgtgttagagaatattggaaaagcgtctgtgagccccgtgctgtattttgtaataaagtcttttgcagattgcttcccgaaaa

SEQ ID NO: 14 - DNA, 3125 bp, human
gcccagttggagccagacagcggggtggacaagtggcgtgtgtgctgcgaccccgagggaagatgaacgggacgcggaactggtgtaccctggtggacgtgcacccagaggaccaggcggcgggcagcgtggacattctcaggctgactctccagggtgaactgacaggagatgaacttgaacacatagcccagaaggcgggcaggaagacctatgccatggtgtccagccactcagctggtcattctctggcttcagaactggtggagtcccatgatggacatgaggagatcattaaggtgtacttgaaggggaggtctggagacaagatgattcacgagaagaatattaaccagctgaagagtgaggtccagtacatccaggaggccaggaactgcctacagaagctccgggaggatataagtagcaagcttgacaggaacctaggagattctctccatcgacaggagatacaggtggtgctagaaaagccaaatggctttagtcagagtcccacagccctgtacagcagcccacctgaggtggacacctgtataaatgaggatgttgagagcttgaggaagacggtgcaggacttgctggccaagcttcaggaggccaagcggcaacaccagtcagactgtgtggcttttgaggtcacactcagccggtaccagagggaagcagaacaaagtaatgtggcccttcagagagaggaggacagagtggagcagaaagaggcagaagtcggagagctgcagaggcgcttgctagggatggagacggagcatcaggccttactggcgaaagtgagggaaggggaggtggccctagaggaacttcggagcaacaatgctgactgccaagcagaacgagaaaaggctgctaccctggaaaaggaagtggccgggttgcgggagaagatccaccacttggatgacatgctcaagagccagcagcggaaagtccggcaaatgatagagcagctccagaattcaaaagctgtgatccagtcaaaggacgccaccatccaggagctcaaggagaaaatcgcctatctggaggcagagaatttagagatgcatgaccggatggaacacctgatagaaaaacaaatcagtcatggcaacttcagcacccaggcccgggccaagacagagaacccgggcagtattaggatatccaagccgcctagcccgaagcccatgcctgtcatccgagtggtggaaacctgagctgcctggagatggttgctgccattgctgctgcctctgcctcggagaagcccactgcccctgttggctgttaacactgcctttgacttcctgactgtcccctggctgcacccaggacttcgggctcctgtgtctcaccattcccaagcccctggccactctaagctgggcagacggagcacgagcacctattcaaggcactgcagccctttggaagacattgtcctgcaagcaggagccagggcaatatctatattcctacagtgactatttttctctgtagagagcctcccttctgttgtagactggactctggctgcgccataagccaggccttcatcagattgggagaggtgacaagatttgcctcagccctaaaagctggagacacagatgtccagagtgattggagaatgtcctgggggaatgaagttccttccacaaacacagctcagttcttagcaacaaactgtttgtttttctacttgctccatctgcagcctacgctgccctggcctcctgcagacagatagtggggttacctggcaaggcctggtgagagccagtgaacctaagctttgactgggtggccttgtctttctggggaggagggaatgtacattcagggagtagccttttgcggaaaaattctctagggctacagacagtcatgtgtgacttctctctgctgtgaaaactcccagagtctctttagggattttccctaaggtgtaccaccaggcacacctcagtcttcttgacccagagcctgaaaactgttttcactgggttccaccagtcccagcaaaatcctctttgtatttattttgctaagttattggtggttttgcttacatctcatgattgatataataccaaagttctatagccttctcttgcagtatttggatttgcttgaaaccgggaaaactgttcccattaggcttgttaatgtcagagtgacactattatgaatctttctctccctttcctctgcctgtttcttctctctttctccttcaaacttgctctgcagctaaggaaggtgagtctactttccctgaggctttggggtcagagtatatgttgtttggagaaagagggcaatcaggactcttctgggacccagatgagttcttcactagcccttctgaaccccttgctccataattggtcttttatcctggctctgaatgaccctgcaggtcatcatggttttctttttttattgtttttttttttctgagacagagtctcactctgtcacccaggctggagtgcagtagcgcgatctcagctcactgcaacctctgcctcccggatttaagcgattcttctgcctcagcctcccgagtagctgggactacaggtgtgccaccacgcctggctgatttttgtatttttagtagagatggggtttcaccatactggctaggctggtctcgaattcctgacctcaggtgatccacccacctcggcttcccaaagtgctaggattataggcttgagctactgtgcccggcccatggtgtttttctttagggctcttcctacagccttgagaagtagataggcatcagagtatggtactataggaatcagaaaaattcaaaacaaatgtggattaagtgtttaggctctatgtggctcacgcagccagaatccttaagtctgtgtgtttctgtgtctcaagactgggctcacattctggctttgtccataacaatgctctgggatttcagggagttccctcatttgtaaaatgagggggtcagagcaggtgatatccatgtttcttccctttctgatattgttgtctgtggcatattctttgtatggcgaatttaataaattatattaatgtgtctctttgaaaaaaaaaaaaaaaaa

SEQ ID NO: 15 - DNA, 2528 bp, human
gagttccagcagtccgcgagctgccgtcggctccgcggggggggcgggccgggcaccccggggcgcggaggagcgctcctcgcttctctccttcccccctgccgcactccgccggaccctcccgccggcccgcgccgctgcactcgccctctcctctcgccccccggcaaactttcggcccctccccgcccctcgcccgttattcgtcgtggctcaagcccggccacgccgccccaagggctcctcccgacctcccggcctgccgctccggccactgcgggatccagaaacatgtcgaccacacttctgtccgccttctacgatgtcgacttcttgtgcaagacagagaaatccctggccaacctcaacctgaacaacatgctggacaagaaggcggtggggacgcctgtggccgccgcccccagctcgggcttcgcgccgggattcctccgacggcactcggccagcaacctgcatgcactcgcccaccccgcgcccagccccggcagctgctcgcccaagttcccgggcgccgctaacggcagcagctgcggcagcgcggcggccggcggtccgacctcctacggcacccttaaggagccgtcggggggcggcggcacagccctgctcaacaaggagaacaaattccgggaccgctcgtttagcgagaacggcgatcgcagccagcacctcctgcacctgcagcagcagcagaaggggggcggcggctcccagatcaactccacgcgctacaagaccgagctgtgccggcccttcgaggagagcggcacgtgcaagtacggcgaaaagtgccagttcgcgcatggcttccacgagctgcgcagcctgactcgccatccgaagtacaagaccgagctgtgccgcacctttcataccatcggcttctgcccctatgggccgcgctgccacttcatccacaacgcggacgagcggcggcccgcgccgtcggggggcgcctccggggacctgcgtgcctttggcacgcgcgatgcgttgcacctgggcttcccgcgggagccgcggcccaagttgcaccacagcctcagcttctcgggcttcccgtcgggccaccatcagcccccgggcggcctcgagtcgccgctgctgctcgacagccccacgtcgcgcacgccgccgccgccctcctgctcttcggcctcgtcctgctcctcctccgcctcctcctgttcctcggcctccgcggcctccacgccctcgggcgccccgacatgctgcgcctccgcggcggccgcggctgcggccgctctgctgtacggcaccgggggcgccgaggacctgctggcgccgggggccccgtgcgcggcctgctcgtcggcctcgtgcgccaacaacgccttcgccttcggtccggagctcagcagcctcatcacgccgctcgccatccagacccacaactttgccgccgtggccgccgccgcctactaccgcagtcagcagcagcagcagcagcagggcctggcgccccccgcgcagccgccggcgccgcccagcgcgaccctccccgccggggccgccgcacctccctcgccgcccttcagcttccagctgccgcgccgcctgtccgactcgcccgtgttcgacgcgccccccagccccccggactcgctgtcggaccgcgacagctacctaagcggctccctgagctccggcagcctcagcggctctgagtctcccagcctcgaccctggccgccgcctgccaatcttcagccgcctctccatctccgacgactgaggcaagagggcgccagtgaggaggaagggaaggcggttcagagatgttggaggacacccctcgccatctcgcccttgctgggggcacgggagtggggggggtgacatgggccctaggcagactgcaagcccgaccgagcacttggactcgaactctgtgccgggaggggcccccacccctcctttttcggtttcctcttgtctttttttttttatttttattacgaagtttcattctttttgagcaaaaaagtcgaactttttctgttgaacaaaatattcacaacagggcagttgtgatacgaatagaacaaaaaaaaaaaaaaaacacttaaactttgttaggactccgatgagtttgggacttcaggaaaaatcaacccagcaccagcagctaccaaccaccattccatctcttcacttgaacagcattagttaagtccagatgtgggaacccttctcttggaagaagttcctaattgtgtctcagaccggtgtaaacaaaccagccagccgccaccttgctaaacctataagctttttaaaatccaatatattctgccaagaatatgccttgatagttagccctcagcccataggtgttttttgttttttaacagaattatatatgtctgggggtgaaaaaacccttgcattccaaaggtccatactggttacttggtttcattgccaccacttagtggatgttcagtttagaaccattttgtctgctccctctggaagccttgcgca

SEQ ID NO: 16 - DNA, 1647 bp, human
gcgcgcgggtttcgttgacccgcggcgttcacgggaattgttcgctttagtgccggcgccatggggtcggagctgatcgggcgcctagccccgcgcctgggcctcgccgagcccgacatgctgaggaaagcagaggagtacttgcgcctgtcccgggtgaagtgtgtcggcctctccgcacgcaccacggagaccagcagtgcagtcatgtgcctggaccttgcagcttcctggatgaagtgccccttggacagggcttatttaattaaactttctggtttgaacaaggagacatatcagagctgtcttaaatcttttgagtgtttactgggcctgaattcaaatattggaataagagacctagctgtacagtttagctgtatagaagcagtgaacatggcttcaaagatactaaaaagctatgagtccagtcttccccagacacagcaagtggatcttgacttatccaggccacttttcacttctgctgcactgctttcagcatgcaagattctaaagctgaaagtggataaaaacaaaatggtagccacatccggtgtaaaaaaagctatatttgatcgactgtgtaaacaactagagaagattggacagcaggtcgacagagaacctggagatgtagctactccaccacggaagagaaagaagatagtggttgaagccccagcaaaggaaatggagaaggtagaggagatgccacataaaccacagaaagatgaagatctgacacaggattatgaagaatggaaaagaaaaattttggaaaatgctgccagtgctcaaaaggctacagcagagtgatttcagcttccaaactggtatacattccaaactgatagtacattgccatctccaggaagacttgacggctttgggattttgtttaaacttttataataaggatcctaagactgttgcctttaaatagcaaagcagcctacctggaggctaagtctgggcagtgggctggcccctggtgtgagcattagaccagccacagtgcctgattggtatagccttatgtgctttcctacaaaatggaattggaggccgggcgcagtggctcacgcctgtaatcccagcactttgggaggccaaggtgggtggatcacctgaggtcaggagctcgagaccagcctggccaacatggtgaaaccccatctctactaaaaatacaaaaattagccaggtgtgatggtgcatgcctgtaatcccagctcctcagtaggctgagacaggagcatcacttgaacgtgggaggcagaggttgcagtgagccgagattgcaccaccgcactccagcctgggtgacagagcgagacttatctcataaataaatagatagatactccagcctgggtgacagagcgagacttatagatagatagatagatagatggatagatagatagatagatagatagatagataaacggaattggagccattttgctttaagtgaatggcagtcccttgtcttattcagaatataaaattcagtctgaatggcatcttacagattttacttcaatttttgtgtacggtattttttatttgactaaatcaatatattgtacagcctaagttaataaatgttatttatatatgcaaaaaaaaaaaaaaaaaaaa

SEQ ID NO: 17 - DNA, 3947 bp, human
aggggcagaggaaacactttccacctcttggatatggattttatcctgccctgattcaggagatccccatctgttaagtaaggggaaagatgattttgttgtcttttgtttcagattctaatgtaggaactggtgagaagaaggtgactgaagcctggatttctgaggatgaaaactcacataggacgacgtcagacagactcacggtgatggagctcccctctcccgagtctgaggaagtccacgagcccagattaggggagctcttgggaaatccagaaggtcagagcctggggagttccccctctcaggacaggggctgcaagcaggtgacagtgacccattggaagatccagacaggagagacagctcaagtgtgcaccaagtcaggaagaaaccatattctgaactcagaccttcttctgcttcagagagagctcatagagggggaagccaatccttgcgatatctgtggcaaaaccttcacgtttaattcggacctagttaggcatcggatttcgcatgctggggagaaaccttacacgtgcgatcagtgtgggaaaggctttggccagagctcacaccttatggagcatcagagaattcacactggagagagactctacgtctgtaatgtgtgtgggaaagacttcattcactattcaggtctcattgagcatcagcgcgttcattcaggagaaaagcccttcaaatgtgcgcagtgtgggaaggcgttttgtcacagttcagacctgattaggcaccagagagttcacaccagagagagaccttttgaatgcaaagagtgtgggaaaggcttcagtcagagctccttacttattcgccatcagaggattcacacgggagaaaggccctatgagtgcaatgaatgtgggaaatccttcataaggagctcgagcctcattcgccattatcagatccacacagaagtgaaacagtatgaatgcaaagaatgtgggaaggcattccgtcatcgctcagaccttattgaacaccagagaattcacaccggagagagaccctttgaatgcaatgagtgtgggaaagcctttattcggagttcaaagctcattcagcatcagaggatccatactggggagaggccttacgtatgcaatgagtgtgggaagcgcttcagccagacgtcaaacttcacccagcatcagagaattcacactggagagaaactctatgaatgtaacgagtgtgggaaagctttctttctgagttcataccttattcgacaccagaaaatccacactggagagagagtgtatgaatgtaaggaatgtgggaaagcgtttctccagaaagcccatctcactgagcaccagaagatccactctggggacaggcccttcgaatgtaaagactgtgggaaagccttcatccagagctccaagctgcttctgcaccagattattcacactggagaaaagccctatgtgtgcagttattgtgggaaaggctttattcagaggtcaaacttccttcaacaccagaaaattcatactgaagagaagctctatgaatgtagtcagtatgggagagattttaactcaactacaaacgttaaaaataatcaaagggttcaccaagagggactctccttgagtaaggcccccatacatttgggtgagaggtctgtagataagggggaacacacaggtaacttataaaataattactttcccgcccagtgagtgatgtttggaaatgcgtggaattaggattcatgtggtttctaagatttggacatgtcagaattttgtgagtcatggatggggctgcttttgcagtgggtgccacctgccactgtgcagccctactcggctcagcccttctcctcagctgtgagcactgtcctcaggagagtcacagggcttgacacctgactctgagctggaacagtaggggcagggagaagacaggtctcaagaaaaggtttttaagaagtttcatccccagttaagcagagtccatccttgacttaaatcccttattacagcacaactgtgtatctaatcttacgatttaggagaatgttacctaggacattttgatgtgttaagttgaagaaaggtaactcgtgtatgaaccccgagccatttccctgttgtcctgaggaggaactccaggcctcccatcgtgtgccctaaggcctcctgcgtcctggagccctgcctcccactgcctgacttcctgccacacggttaatgctgcagcaacaccgactgcttcatcttccctgtgctccacgtggcttcctacctctctcgcctttgttcttgttgaagggtctcttctcagctaattaactctgaatcatggttcaagacaagcctcaggcatcatgtcaatgggtgtttccctcaagcttagttggcagcactctccacacttctgtggctcagtgattactgctattactatatttacttgcatatgtcagaatgatgtgatagactatctctgtcactatgctgttgggttcctgaggacagtgatcatatctgattgatttccatgtgtccactgtctagcacagggcaataaaaaatacacccctaaatctatgtgtaattggcatctctcttgctttgtcctttctatactgccattctaaaaattttcagctgttggctgttttttttgttgtttgttcattttgtatcagtataatctacgattctgttagaagtatcttctcagccctgctactgtctgctgctcctacttagaagttgcaggcaatatcctgtgcacatccacgccctcctgtgtgccaccttacagctcatgcagaactgtctacttgatctggagggcattgacacctggctcagctggaacagtggggacaggaggaggcaggtctcaagagaaggtctttacttacttgtctgtatctttctgtctcactcgaaaggcacagtctcaatgcgctacgtaactcttctgttgtagcagttctgaggttgaactgagctcttgtcattttccatgctctctggctcattgtgtcatttcaaaatgtattgatatctataaggaatagtcctcaatgttgggaaaacagatgagcaacccactaccctgaagtcatccaccgtgtagtggggaagacggagatcacagtagggtgagttaagtatgtgccctcaggtgtgaaggagcacaggtgagggggtgcctgctgtggcacagagctgggaaggccttacagaagaggtgacatttcagctgggccagagatcagtagagagcagctgaagaagaaaatagggtccaggagaggaatctgcagaaacatggagccatgacagatggctttttggctaggactgttcgaaagcaggcgaggtaaatggacagtggacagccgcctcgccaaactgttagtctttacctctgaatttcatctggacatgaaccccaaagtccagttttgtaacgatataatttttttctcatgaggccatattttgagttcttaaatactaccaaccctgaacgtctcaggacaaataattcaaaaaagagatctacattttctggaaaatcagcattactagagatgtttcaaatgtagattttaccagaccattttaatcaactttattgagattcaattttcatatagtaaaacgcctatttaagtgtacaaggttttgaaaaatctatacacctgtgtaaccaccaccacctgtgtaaccaccaccgccagaaccaaaaacgtttctgtcacccaaagaagttcccttttgccttttcctagttaagcccactccagccccagataaccactttctatcctcatagattagtgttgcttggtttagagcctcatacaaatggattcatttactatgtgttcttttgcgtctgatgtctcactttggcatgtttttagactcatccaagct

SEQ ID NO: 18 - DNA, 6641 bp, human
ggttacattttggatcctcgcggagtactggtcaggcggttaagtcctgtacctaggaaagagggcgagctctggggcgctctccggtgtcatgaggagcttcaaaagagtcaactttgggactctgctaagcagccagaaggaggctgaagagttgctgcccgccttgaaggagttcctgtccaaccctccagctggttttcccagcagccgatctgatgctgagaggagacaagcttgtgatgccatcctgagggcttgcaaccagcagctgactgctaagctagcttgccctaggcatctggggagcctgctggagctggcagagctggcctgtgatggctacttagtgtctaccccacagcgtcctcccctctacctggaacgaattctctttgtcttactgcggaatgctgctgcacaaggaagcccagaggccacactccgccttgctcagcccctccatgcctgcttggtgcagtgctctcgcgaggctgctccccaggactatgaggccgtggctcggggcagcttttctctgctttggaagggggcagaagccctgttggaacggcgagctgcatttgcagctcggctgaaggccttgagcttcctagtactcttggaggatgaaagtaccccttgtgaggttcctcactttgcttctccaacagcctgtcgagcggtagctgcccatcagctatttgatgccagtggccatggtctaaatgaagcagatgctgatttcctagatgacctgctctccaggcacgtgatcagagccttggtgggtgagagagggagctcttctgggcttctttctccccagagggccctctgcctcttggagctcaccttggaacactgccgtcgcttttgctggagccgccaccatgacaaagccatcagcgcagtggagaaggctcacagttacctaaggaacaccaatctagcccctagccttcagctatgtcagctgggggttaagctgctgcaggttggggaggaaggacctcaggcagtggccaagcttctgatcaaggcatcagctgtcctgagcaagagtatggaggcaccatcacccccacttcgggcattgtatgagagctgccagttcttcctttcaggcctggaacgaggcaccaagaggcgctatagacttgatgccattctgagcctctttgcttttcttggagggtactgctctcttctgcagcagctgcgggatgatggtgtgtatgggggctcctccaagcaacagcagtcttttcttcagatgtactttcagggacttcacctctacactgtggtggtttatgactttgcccaaggctgtcagatagttgatttggctgacctgacccaactagtggacagttgtaaatctaccgttgtctggatgctggaggccttagagggcctgtcgggccaagagctgacggaccacatggggatgaccgcttcttacaccagtaatttggcctacagcttctatagtcacaagctctatgccgaggcctgtgccatctctgagccgctctgtcagcacctgggtttggtgaagccaggcacttatcccgaggtgcctcctgagaagttgcacaggtgcttccggctacaagtagagagtttgaagaaactgggtaaacaggcccagggctgcaagatggtgattttgtggctggcagccctgcaaccctgtagccctgaacacatggctgagccagtcactttctgggttcgggtcaagatggatgcggccagggctggagacaaggagctacagctaaagactctgcgagacagcctcagtggctgggacccggagaccctggccctcctgctgagggaggagctgcaggcctacaaggcggtgcgggccgacactggacaggaacgcttcaacatcatctgtgacctcctggagctgagccccgaggagacaccagccggggcctgggcacgagccacccacctggtagaactggctcaggtgctctgctaccacgactttacgcagcagaccaactgctctgctctggatgctatccgggaagccctgcagcttctggactctgtgaggcctgaggcccaggccagagatcagcttctggacgataaagcacaggccttgctgtggctttacatctgtactctggaagccaaaatgcaggaaggtatcgagcgggatcggagagcccaggcccctggtaacttggaggaatttgaagtcaatgacctgaactatgaagataaactccaggaagatcgtttcctatacagtaacattgccttcaacctggctgcagatgctgctcagtccaaatgcctggaccaagccctggccctgtggaaggagctgcttacaaaggggcaggccccagctgtacggtgtctccagcagacagcagcctcactgcagatcctagcagccctctaccagctggtggcaaagcccatgcaggctctggaggtcctcctgctgctacggattgtctctgagagactgaaggaccactcgaaggcagctggctcctcctgccacatcacccagctcctcctgaccctcggctgtcccagctatgcccagttacacctggaagaggcagcatcgagcctgaagcatctcgatcagactactgacacatacctgctcctttccctgacctgtgatctgcttcgaagtcaactctactggactcaccagaaggtgaccaagggtgtctctctgctgctgtctgtgcttcgggatcctgccctccagaagtcctccaaggcttggtacttgctgcgtgtccaggtcctgcagctggtggcagcttaccttagcctcccgtcaaacaacctctcacactccctgtgggagcagctctgtgcccaaggctggcagacacctgagatagctctcatagactcccataagctcctccgaagcatcatcctcctgctgatgggcagtgacattctctcaactcagaaagcagctgtggagacatcgtttttggactatggtgaaaatctggtacaaaaatggcaggttctttcagaggtgctgagctgctcagagaagctggtctgccacctgggccgcctgggtagtgtgagtgaagccaaggccttttgcttggaggccctaaaacttacaacaaagctgcagataccacgccagtgtgccctgttcctggtgctgaagggcgagctggagctggcccgcaatgacattgatctctgtcagtcggacctgcagcaggttctgttcttgcttgagtcttgcacagagtttggtggggtgactcagcacctggactctgtgaagaaggtccacctgcagaaggggaagcagcaggcccaggtcccctgtcctccacagctcccagaggaggagctcttcctaagaggccctgctctagagctggtggccactgtggccaaggagcctggccccatagcaccttctacaaactcctccccagtcttgaaaaccaagccccagcccatacccaacttcctgtcccattcacccacctgtgactgctcgctctgcgccagccctgtcctcacagcagtctgtctgcgctgggtattggtcacggcaggggtgaggctggccatgggccaccaagcccagggtctggatctgctgcaggtcgtgctgaagggctgtcctgaagccgctgagcgcctcacccaagctctccaagcttccctgaatcataaaacacccccctccttggttccaagcctcttggatgagatcttggctcaagcatacacactgttggcactggagggcctgaaccagccatcaaacgagagcctgcagaaggttctacagtcagggctgaagtttgtagcagcacggataccccacctagagccctggcgagccagcctgctcttgatttgggccctcacaaaactaggtggcctcagctgctgtactacccaactttttgcaagctcctggggctggcagccaccattaataaaaagtgtccctggctcagagccctctaagactcagggccaaaaacgttctggacgagggcgccaaaagttagcctctgctcccctgcgcctcaataatacctctcagaaaggtctggaaggtagaggactgccctgcacacctaaacccccagaccggatcaggcaagctggccctcatgtccccttcacggtgtttgaggaagtctgccctacagagagcaagcctgaagtaccccaggcccccagggtacaacagagagtccagacgcgcctcaaggtgaacttcagtgatgacagtgacttggaagaccctgtctcagctgaggcctggctggcagaggagcctaagagacggggcactgcttcccggggccgggggcgagcaaggaagggcctgagcctaaagacggatgccgtggttgccccaggtagtgcccctgggaaccctggcctgaatggcaggagccggagggccaagaaggtggcatcaagacattgtgaggagcggcgtccccagagggccagtgaccaggccaggcctggccctgagatcatgaggaccatccctgaggaagaactgactgacaactggagaaaaatgagctttgagatcctcaggggctctgacggggaagactcagcctcaggtgggaagactccagctccgggccctgaggcagcttctggagaatgggagctgctgaggctggattccagcaagaagaagctgcccagcccatgcccagacaaggagagtgacaaggaccttggtcctcggctccggctcccctcagcccccgtagccactggtctttctaccctggactccatctgtgactccctgagtgttgctttccggggcattagtcactgtcctcctagtgggctctatgcccacctctgccgcttcctggccttgtgcctgggccaccgggatccttatgccactgctttccttgtcaccgagtctgtctccatcacctgtcgccaccagctgctcacccacctccacagacagctcagcaaggcccagaagcaccgaggatcacttgaaatagcagaccagctgcaggggctgagccttcaggagatgcctggagatgtccccctggcccgcatccagcgcctcttttccttcagggctttggaatctggccacttcccccagcctgaaaaggagagtttccaggagcgcctggctctgatccccagtggggtgactgtgtgtgtgttggccctggccaccctccagcccggaaccgtgggcaacaccctcctgctgacccggctggaaaaggacagtcccccagtcagtgtgcagattcccactggccagaacaagcttcatctgcgttcagtcctgaatgagtttgatgccatccagaaggcacagaaagagaacagcagctgtactgacaagcgagaatggtggacagggcggctggcactggaccacaggatggaggttctcatcgcttccctagagaagtctgtgctgggctgctggaaggggctgctgctgccgtccagtgaggagcccggccctgcccaggaggcctcccgcctacaggagctgctacaggactgtggctggaaatatcctgaccgcactctgctgaaaatcatgctcagtggtgccggtgccctcacccctcaggacattcaggccctggcctacgggctgtgcccaacccagccagagcgagcccaggagctcctgaatgaggcagtaggacgtctacagggcctgacagtaccaagcaatagccaccttgtcttggtcctagacaaggacttgcagaagctgccgtgggaaagcatgcccagcctccaagcactgcctgtcacccggctgccctccttccgcttcctactcagctactccatcatcaaagagtatggggcctcgccagtgctgagtcaaggggtggatccacgaagtaccttctatgtcctgaaccctcacaataacctgtcaagcacagaggagcaatttcgagccaatttcagcagtgaagctggctggagaggagtggttggggaggtgccaagacctgaacaggtgcaggaagccctgacaaagcatgatttgtatatctatgcagggcatggggctggtgcccgcttccttgatgggcaggctgtcctgcggctgagctgtcgggcagtggccctgctgtttggctgtagcagtgcggccctggctgtgcgtggaaacctggagggggctggcatcgtgctcaagtacatcatggctggttgccccttgtttctgggtaatctctgggatgtgactgaccgcgacattgaccgctacacggaagctctgctgcaaggctggcttggagcaggcccaggggccccccttctctactatgtaaaccaggcccgccaagctccccgactcaagtatcttattggggctgcacctatagcctatggcttgcctgtctctctgcggtaaccccatggagctgtcttattgatgctagaagcctcataactgttctacctccaaggttagatttaatccttaggataactcttttaaagtgattttccccagtgttttatatgaaacatttccttttgatttaacctcagtataataaagatacatcatttaaaccctgaaaaaaaaaaaaaaaaaa

SEQ ID NO: 19 - DNA, 668 bp, human
cggggacccggctgcggtggctgcgggactgacgagaaactactaaagttcctggggaagcaaagtagaatttcataagaacaaaatggatggagaggagaaaacctatggtggctgtgaaggacctgatgccatgtatgtcaaattgatatcatctgatggccatgaatttattgtaaaaagagaacatgcattaacatcaggcacgataaaagccatgttgagtggcccaggtcagtttgctgagaacgaaaccaatgaggtcaattttagagagataccttcacatgtgctatcgaaagtatgcatgtattttacgtacaaggttcgctacactaacagctccaccgagattcctgaattcccaattgcacctgaaattgcactggaactgctgatggctgcgaacttcttagattgttaaataaaataaattataataaactgttaactcttttcagtatttaatacctgtagttcagttagtaactttttcatatatagcatgttgcctgtatgcagttgaactatataaagttcattgcaaagcagattatcttgtttttttgcatagcaatcaaagttgaaatttgtttgctacatcaacaaattaaggacattttcacaaactgagaaataaacaaatatgccaattaaaaaaaaaaaaa

SEQ ID NO: 20 - DNA, 5332 bp, human
gctgaactttaggagccagtctaaggcctaggcgcagacgcactgagcctaagcagccggtgatggcggcagcggctgtggtggctgcggcgggtccgggcccatgaggcgacgaaggaggcgggacggcttttacccagccccggacttccgagacagggaagctgaggacatggcaggagtgtttgacatagacctggaccagccagaggacgcgggctctgaggatgagctggaggaggggggtcagttaaatgaaagcatggaccatgggggagttggaccatatgaacttggcatggaacattgtgagaaatttgaaatctcagaaactagtgtgaacagagggccagaaaaaatcagaccagaatgttttgagctacttcgggtacttggtaaagggggctatggaaaggtttttcaagtacgaaaagtaacaggagcaaatactgggaaaatatttgccatgaaggtgcttaaaaaggcaatgatagtaagaaatgctaaagatacagctcatacaaaagcagaacggaatattctggaggaagtaaagcatcccttcatcgtggatttaatttatgcctttcagactggtggaaaactctacctcatccttgagtatctcagtggaggagaactatttatgcagttagaaagagagggaatatttatggaagacactgcctgcttttacttggcagaaatctccatggctttggggcatttacatcaaaaggggatcatctacagagacctgaagccggagaatatcatgcttaatcaccaaggtcatgtgaaactaacagactttggactatgcaaagaatctattcatgatggaacagtcacacacacattttgtggaacaatagaatacatggcccctgaaatcttgatgagaagtggccacaatcgtgctgtggattggtggagtttgggagcattaatgtatgacatgctgactggagcacccccattcactggggagaatagaaagaaaacaattgacaaaatcctcaaatgtaaactcaatttgcctccctacctcacacaagaagccagagatctgcttaaaaagctgctgaaaagaaatgctgcttctcgtctgggagctggtcctggggacgctggagaagttcaagctcatccattctttagacacattaactgggaagaacttctggctcgaaaggtggagcccccctttaaacctctgttgcaatctgaagaggatgtaagtcagtttgattccaagtttacacgtcagacacctgtcgacagcccagatgactcaactctcagtgaaagtgccaatcaggtctttctgggttttacatatgtggctccatctgtacttgaaagtgtgaaagaaaagttttcctttgaaccaaaaatccgatcacctcgaagatttattggcagcccacgaacacctgtcagcccagtcaaattttctcctggggatttctggggaagaggtgcttcggccagcacagcaaatcctcagacacctgtggaatacccaatggaaacaagtggcatagagcagatggatgtgacaatgagtggggaagcatcggcaccacttccaatacgacagccgaactctgggccatacaaaaaacaagcttttcccatgatctccaaacggccagagcacctgcgtatgaatctatgacagagcaatgcttttaatgaatttaaggcaaaaaaggtggagagggagatgtgtgagcatcctgcaaggtgaaacgactcaaaatgacagtttcagagagtcaatgtcattacatagaacacttcagacacaggaaaaataaacgtggattttaaaaaatcaatcaatggtgcaaaaaaaaacttaaagcaaaatagtattgctgaactcttaggcacatcaattaattgattcctcgcgacatcttctcaaccttatcaaggattttcatgttgatgactcgaaactgacagtattaagggtaggatgttgcttctgaatcactgttgagttctgattgtgttgaagaagggttatcctttcattaggcaaagtacaaaattgcctataatacttgcaactaaggacaaattagcatgcaagcttggtcaaactttttccagcaaaatggaagcaaagacaaaagaaacttaccaattgatgttttacgtgcaaacaacctgaatcttttttttatataaatatatatttttcaaatagatttttgattcagctcattatgaaaaacatcccaaactttaaaatgcgaaattattggttggtgtgaagaaagccagacaacttctgtttcttctcttggtgaaataataaaatgcaaatgaatcattgttaaccacagctgtggctcgtttgagggattggggtggacctggggtttattttcagtaacccagctgcaatacctgtctgtaatatgagaaaaaaaaaatgaatctatttaatcatttctacttgcagtactgctatgtgctaagcttaactggaagccttggaatgggcataagttgtatgtcctacatttcatcattgtcccgggcctgcattgcactggaaaaaaaaatcgccacctgttcttacaccagtatttggttcaagacaccaaatgtcttcagcccatggctgaagaacaacagaagagagtcaggataaaaaatacatactgtggtcggcaaggtgagggagatagggatatccaggggaagagggtgttgctgtggcccactctctgtctaatctctttacagcaaattggtaagattttcagttttacttctttctactgtttctgctgtctaccttccttatatttttttcctcaacagttttaaaaagaaaaaaaggtctatttttttttctcctatacttgggctacattttttgattgtaaaaatatttgatggccttttgatgaatgtcttccacagtaaagaaaacttagtggcttaatttaggaaacatgttaacaggacactatgtttttgaaattgtaacaaaatctacataaatgatttacaggttaaaagaataaaaataaaggtaactttacctttcttaaatatttcctgccttaaagagagcatttccatgactttagctggtgaaagggtttaatatctgcagagctttataaaaatatatttcagtgcatactggtataatagatgatcatgcagttgcagttgagttgtatcaccttttttgtttgtcttttataatgtcttcagtctgagtgtgcaaagtcaatttgtaatattttgcaaccctaggatttttttaaatagatgctgcttgctatgttttcaaacctttttgagccataggatccaagccataaaattctttatgcatgttgaattcagtcagaaaagagcaaggctttgctttttgaaattgcaactcaaatgagatgggatgaaatcctatgacagtaagcaaaaacagaaccatgaaaaatgattggacatacaccttttcaattgtggcaataattgaaagaatcgataaaagttcatctttggacagaaagcctttaaaaaaaaaatcactccctcttccccctcctcccttattgcagcagcctactgagaactttgactgttgctggtaaattagaagctacaataataattaagggcagaaattatacttaaaaagtgcagatccttgttctttgacaatttgtgatgtctgaaaaaacagaacccgaaaagctatggtgatatgtacaggcattatttcagactgtaaatggcttgtgatactcttgatacttgttttcaaatatgtttactaactgtagtgttgactgcctgaccaaattccagtgaaacttatacaccaaaatattcttcctaggtcctatttgctagtaacatgagcactgtgattggctggctataaccaccccagttaaaccattttcataattagtagtgccagcaatagtggcaaacactgcaacttttctgcataaaaagcattaattgcacagctaccatccacacaaatacatagtttttctgacttcacatttattaagtgaaatttatttcccatgctgtggaaagtttattgagaacttgtttcataaatggatatccctactatgactgtgaaaacatgtcaagtgtcacattagtgtcacagacagaaagcacacacctatgcaatatggcttatctatatttatttgtaaaaatccaagcatagtttaaaatatgatgtcgatattactagtcttgagtttctaagagggttctttatgttataccaggtaagtgtataaaagagattaagtgcttttttttcatcacttgattattttctttaaaatcagctattacaggatatttttttattttatacatgctgttttttaattaaaatataatcactgaagtttactaatttgattttataaggtttgtagcattacagaataactaaactgggatttataaaccagctgtgattaacaatgtaaagtattaattattgaactttgaaccagatttttaggaaaattatgttctttttccccctttatggtcttaactaatttgaatccttcaagaaggatttttccatactattttttaagatagaagataatttgtgggcaggggtggaggatgcatgtatgatactccataaattcaacattctttactataggtaatgaatgattataaacaagatgcatcttagatagtattaatatactgagccttggattatatatttaatataggacctattttgaatattcagttaatcatatggttcctagcttacaagggctagatctaagattattcccatgagaaatgttgaatttatgaagaatagattttaaggctttgaaaatggttaatttctcaaaaacatcaatgtccaaacatctaccttttttcataggagtagacactagcaagctggacaaactatcacaaaagtatttgtcacacataacctgtggtctgttgctgattaatacagtactttttcttgtgtgattcttaacattatagcacaagtattatctcagtggattatccggaataacatctgaaagatgggttcatctatgtttgtgtttgctctttaaactattgtttctcctatcccaagttcgctttgcatctatcagtaaataaaattcttcagctgccttattaggagtgctatgagggtaacacctgttctgcttttcatcttgtatttagttgactgtattatttgatttcggattgaatgaatgtaaatagaaattaaatgcaaatttgaatgaacataaaaaaaaaaaaaaaa

SEQ ID NO: 21 - DNA, 4507 bp, human
tcagcggcagcagccgccgccgagatgtcccggcgaaagcaaagcaaaccccggcagatcaaacggccgcttgaagatgccattgaagatgaggaagaagaatgtccatcagaggaaacagacatcatctccaaaggagactttccattggaggaaagcttttccacagaatttgggcctgaaaatctgagctgcgaagaagtggaatacttttgtaacaaaggtgatgatgaaggaatccaggagacagcagaatcagatggggacacacagtcagagaaaccggggcaacctggagttgagacagacgactgggatggaccaggagagctggaggtgtttcagaaagatggggaacgaaaaattcagagtcgacagcaacttccagtgggaacaacctgggggccgtttcctgggaagatggacttgaataataattctttgaagacaaaggctcaggtcccaatggtgctgactgctggtcccaagtggttgctggatgtgacttggcaaggagtggaagacaacaaaaacaactgcattgtgtacagcaaagggggtcagctttggtgtacaactacgaaggccatctctgagggtgaagagctaattgcctttgtggtggattttgactcaaggctacaagctgccagtcagatgactctcacagaagggatgtaccctgcacgcctgctggactcaattcagctgcttcctcagcaagctgccatggcttctattttgcccacagctattgtcaataaggatatattcccttgcaagtcctgtggcatctggtatcggagtgagcggaatctgcaggcccatttgatgtactactgcagtgggaggcaaagagaagctgctccggtgtcagaggaaaatgaagacagtgcccatcagatttccagcctgtgccccttcccacagtgcaccaagagcttttcaaatgctcgagctctagaaatgcacctgaattcacacagtggagtgaaaatggaagaattcctgccccctggtgctagtctaaaatgcaccgtctgtagctacactgctgattccgtgatcaactttcaccaacacctgttctcccatctcactcaagctgccttccgatgtaatcactgccatttcggcttccagactcagagggagttattgcagcaccaggagctccatgtccctagcggcaaacttcccagagaaagtgacatggaacactctccaagtgcaactgaagacagcttacagccagccacagacttattgaccagaagcgaacttccccagagccaaaaggccatgcagactaaagatgcgagctctgacacagagctggacaagtgtgagaaaaagactcagctctttctcacgaaccagagaccagagatacagcctacaacaaataaacaaagcttttcttacacaaaaataaagtctgagccctctagcccaagacttgcctcatctccagttcagcctaatattgggccttctttccctgtgggccctttcctatctcagttttctttcccccaagatatcaccatggtccctcaagcttcagagatcttagctaagatgtctgaactggtgcatcggcgactgaggcatggcagtagtagctaccctcccgtcatttacagccctttgatgcccaagggggctacttgttttgagtgtaacataacattcaataatttggataattatctagtgcacaaaaagcattattgcagcagccgatggcagcagatggctaagtccccagagttccctagtgtgtcagaaaagatgcctgaagctttgagtcccaacactggccaaacctccataaaccttctcaacccagctgctcattctgctgatcctgagaatccacttcttcaaacatcttgcatcaattcttccactgtcttagatttaattgggccaaatgggaagggccatgacaaggacttttccactcaaactaagaagctctccacctccagtaacaatgatgacaaaattaatggaaaacctgttgatgtgaaaaatcccagtgtccccttagtggatggggaaagtgacccaaataagactacctgtgaagcttgcaacattaccttcagccggcacgaaacatacatggtccacaaacagtattactgtgctacacgccacgaccctccactgaagaggtctgcttccaacaaagtgcctgccatgcagagaaccatgcgcacacgcaagcgcagaaagatgtatgagatgtgcctacctgagcaggaacaaaggcctccactggttcagcagagatttcttgacgtagccaacctcaataatccttgtacctccactcaagaacccacagaagggctaggagagtgctaccacccaagatgtgatatctttccaggaattgtctctaaacacttggaaacttctctgacgatcaacaagtgtgttccagtttccaaatgtgatactactcattccagtgtttcctgcctagagatggacgtgcccatagatctcagcaaaaagtgtttatctcagtctgagcggacgaccacgtctcccaaaaggctgctggactatcacgagtgcactgtgtgcaagatcagtttcaataaggtagaaaactatctggcccacaagcagaatttctgcccggttactgcacatcagcgtaatgacctgggtcaactggacggcaaagtgtttccgaatccagaaagcgaacgaaacagccctgatgtcagctacgaaagaagcataataaaatgtgagaaaaatgggaatttgaagcagccttcccccaatggaaacttattttcatcccacctagcaaccccgcaaggcttgaaggtctttagtgaagctgctcagctcattgctacaaaagaagaaaacagacatttgtttcttccacaatgcctttaccctggagcaataaagaaagcaaaaggagccgaccagctttctccatattatggaatcaagccaagtgattatatttctggttctcttgtcatccataacactgacatcgagcaaagcagaaatgcagaaaatgaatctcctaaaggccaggcttcctcaaatgggtgtgctgcgctgaagaaagattctctgccattgttgcccaaaaatcgaggaatggtaatagtgaatggtggactgaaacaagatgagagacctgctgccaacccacagcaagagaacatttcccagaatcctcagcacgaagacgaccacaaatctccctcgtggatctctgagaacccattagctgccaatgagaatgtctcaccaggaattccctcagcagaggaacagttgtctagtatagcaaaaggtgtgaatggttccagccaggctccaaccagtgggaaatattgccggctatgtgatatccagttcaacaacctttcaaactttataactcacaagaagttttattgctcatcacatgcagcagaacatgtcaaatgaactaactaaacatcagtcacctttggtatcagtgtttagtatgttgttctaaccagtccagaaaaaaaaataagctgtttgaattacatctgggcaatcaggagataattcattatggctgagttgaagacttaaggtgtaatttcattacagtccattagtaaagtgtattattggtgccattttcaaaaaaattaatttattttaccagcagtattcatagctgtggttatgttattttttatttaaaaactttatattaaagtcatttgtaatgttattgtatagttattgtgtagcacatatggtttgcactgtatagtagcttttaaagaaaatagtcacaatacagaaaagcattttagaaatagcttcaaaagcacttgtgtatcttgattttttcttatatgctgttgcagatatatgtatatgctaaaatataacttgcaaagatgttctaaatacacatgctataagttcgccttaagatttcaattcttggataatcaggctctgtttgcactttatattttagcagatacagtctcttagtcactaggctttgcatttgtatgtagctgtatgtttccgtccattttcttaatcctgaacctgtatgttaaatgaagatggcaatttttttcttgtatagtacttgtattttctttcgctgatgcagctctgtctcaatttttaaacctttgctgttaaatgcaatactttataaagaatgaacaaaattactggaagcagtattgtaagtaatgaggtagtattaatcagttttatcttttgaaaggcacagtctaaatcgaaaccctaaactcaatgctgcaagtatgaatttaattcatatataagatctatttaaatataagagtagcaatactgcacctggtgatcacaaagataatgttctacttctgatagaaataatttctcaacaaatgttgttactatgcatgtatatggatggaataaaattccagattgttggagaaaaaaaaaaaaaaaaaaaaaaaaaaaa

SEQ ID NO: 22 - DNA, 723 bp, human
gcgcactcagggtctgaggcagctagtagccggagggtcaccatgaagttcaatcccttcgttacctcggaccgcagtaaaaaccgcaaacgtcacttcaatgccccctcacacgtgcgcaggaagatcatgtcatccccgctctccaaggagctgcggcagaagtacaatgtccgctccatgcccatccgcaaggacgacgaggtccaggtagttcgaggacactacaaaggtcagcaaattggcaaggtagtccaggtgtacagaaagaaatatgtcatctacatcgagcgggtgcagcgtgagaaggccaacggcacaactgtccacgtgggcattcacccaagcaaggtggttatcaccaggctaaaactggacaaggatcggaaaaaaattcttgaacgcaaagccaagtctcgacaagttggaaaagagaaaggcaaatataaagaagaacttattgagaaaatgcaggaataaatagaacctgttgtgcaaccacggtttaaccggagattttgaggctagggtgtgtttctttcgaacttttcggaatgtctggaacatttcatttcctgttttgttacctgtgcctctgtaaatctacttttgcaattttaagtaataattttatgaataaaaatgggaaatgcttcctaattccacatagtatttgcattgttttataaataaattccacttactatcaaaaaaaaaaaaaaa

SEQ ID NO: 23 - DNA, 765 bp, human
atgacgccgcggaggcggccccgtccccgcgctcccgcccctcccgccagggcagcccgggaggccagacgttgacgctgcagggagagggtggtgggcgcagccgctagggggcgcggcggggcggagcgcacctttccgcgggccgcggggatggcggcgcagggcgtagggcctgggccggggtcggcggcgcccccggggctggaggcggcccggcagaagctggcgctgcggcggaagaaggtgctgagcaccgaggagatggagctgtacgagctggcgcaggcggcgggcggcgctatcgaccccgacgtgttcaagatcctggtggacctgctgaagctgaacgtggcccccctcgccgtcttccagatgctcaagtccatgtgtgccgggcagaggctagcgagcgagccccaggaccctgcggccgtgtctctgcccacgtcgagcgtgcccgagacccgagggagaaacaaaggcagcgctgccctcgggggagcattggccctggcggaacgcagcagccgcgaaggatccagccagaggatgccacgccagcccagcgctaccaggctgcccaaggggggcgggcctgggaagagccctacacggggcagcacctaggatggggcagagacttgttgcatctttgtccccagcaaaggctacatgttacctccttcaattgataataaacctttctgagatgcagagggtccaggtcaaaaaaaaaaaaaaaaaaaaaaaaaaaaaaaaaaa

SEQ ID NO: 24 - DNA, 3608 bp, human
gcggccgcttccccccggccgggcccccgccgccccgcggtccccagagcgccaggcccccggggggagggagggagggcgccgggccggtgggagccagcggcgcgcggtgggacccacggagccccgcgacccgccgagcctggagccgggccgggtcggggaagccggctccagcccggagcgaacttcgcagcccgtcggggggcggcggggagggggcccggagccggaggagggggcggccgcgggcacccccgcctgtgccccggcgtccccgggcaccatgctgtccaactcccagggccagagcccgccggtgccgttccccgccccggccccgccgccgcagccccccacccctgccctgccgcaccccccggcgcagccgccgccgccgcccccgcagcagttcccgcagttccacgtcaagtccggcctgcagatcaagaagaacgccatcatcgatgactacaaggtcaccagccaggtcctggggctgggcatcaacggcaaagttttgcagatcttcaacaagaggacccaggagaaattcgccctcaaaatgcttcaggactgccccaaggcccgcagggaggtggagctgcactggcgggcctcccagtgcccgcacatcgtacggatcgtggatgtgtacgagaatctgtacgcagggaggaagtgcctgctgattgtcatggaatgtttggacggtggagaactctttagccgaatccaggatcgaggagaccaggcattcacagaaagagaagcatccgaaatcatgaagagcatcggtgaggccatccagtatctgcattcaatcaacattgcccatcgggatgtcaagcctgagaatctcttatacacctccaaaaggcccaacgccatcctgaaactcactgactttggctttgccaaggaaaccaccagccacaactctttgaccactccttgttatacaccgtactatgtggctccagaagtgctgggtccagagaagtatgacaagtcctgtgacatgtggtccctgggtgtcatcatgtacatcctgctgtgtgggtatccccccttctactccaaccacggccttgccatctctccgggcatgaagactcgcatccgaatgggccagtatgaatttcccaacccagaatggtcagaagtatcagaggaagtgaagatgctcattcggaatctgctgaaaacagagcccacccagagaatgaccatcaccgagtttatgaaccacccttggatcatgcaatcaacaaaggtccctcaaaccccactgcacaccagccgggtcctgaaggaggacaaggagcggtgggaggatgtcaaggggtgtcttcatgacaagaacagcgaccaggccacttggctgaccaggttgtgagcagaggattctgtgttcctgtccaaactcagtgctgtttcttagaatccttttattccctgggtctctaatgggaccttaaagaccatctggtatcatcttctcattttgcagaagagaaactgaggcccagaggcggagggcagtctgctcaaggtcacgcagctggtgactggttggggcagaccggacccaggtttcctgactcctggcccaagtctcttcctcctatcctgcgggatcactggggggctctcagggaacagcagcagtgccatagccaggctctctgctgcccagcgctggggtgaggctgccgttgtcagcgtggaccactaaccagcccgtcttctctctctgctcccacccctgccgccctcaccctgcccttgttgtctctgtctctcacgtctctcttctgctgtctctcctacctgtcttctggctctctctgtacccttcctggtgctgccgtgcccccaggaggagatgaccagtgccttggccacaatgcgcgttgactacgagcagatcaagataaaaaagattgaagatgcatccaaccctctgctgctgaagaggcggaagaaagctcgggccctggaggctgcggctctggcccactgagccaccgcgccctcctgcccacgggaggacaagcaataactctctacaggaatatattttttaaacgaagagacagaactgtccacatctgcctcctctcctcctcagctgcatggagcctggaactgcatcagtgactgaattctgccttggttctggccaccccagagtgggagaggctgggaggttgggaggctgtggagagaagtgagcaaggtgctcttgaacctgtgctcattttgcaattttatcagtaatttgacttagagtttttacgaaacctcttttgttgtccttgccccactcctctccaccagacgccttcctctctggatactgcaaaggcttgtggtttgttagagggtatttgtggaaactgtcatagggattgtccctgtgttgtcccatctgccctccctgtttctccacaacagcctggggttgtccccgctggctcacgcgttctgggagctcaaggccaccttggaggaggatgccacgcacttcctctctcggagccctcagacatctccagtgtgccagacaaataggagtgagtgtatgtgtgtgtgtgtgtgtgtgtgtgtgcacacgtgtgtatgagtgcgcagatctgtgcctgggatcgtgcatttgaggggccaggggcaggcagggctgcagagggagacggccctgctggggcttaggaaccttctcccttcttgggtctgccctgcccatactgagcctgccaaagtgcctgggaagcccacccagattctgaaacaggccctctgtggcctgtctctattagctgggttccgggaggcagagaggagtgaccgggcactggcactgcgatcaggaagactggacccccagcccccagggcccccctccccccacttagtgctggtcctaggtcctctgaggcactcatctactgaatgacctctctacttccccttcttgccattattaacccatttttgtttattttccttaaatttttagccatttctccatgggccaccgcccagctcatgtaggtgagcctgggcagcttctgttggcagagcttttgcatttcctgtgtttgtcctgggttctggggcatcagccagctaccccttgtgggcaaaggcagggccacttttgaagtcttccctcagatttccattgtgtggcctggtgggtcagggggagtctttgcaccaaagatgtcctgactttgcccccttgcccatcagccatttgccatcaccccaaacaactcagcttcggggccggtgaggggaggggcctcccccagcacagatgaggagcagctggggtaggctgtctgtgccatggccccccactcccccttcccttggagggagaggtggcaggaatacttcacctttcctctccctcaggggcaggtggtggaggggcgcccagggtcgtctttgtgtatgggggaaggcgctgggtgcctgcagcgcctcccttgtctcagatggtgtgtccagcactcgattgttgtaaactgttgttttgtatgagcgaaattgtctttactaaacagatttaatagttaaaaaaaaaaaaaaaaaaa

SEQ ID NO: 25 - DNA, 5087 bp, human
aacgggcgccgcggcggggagaagacgcagagcgctgctgggctgccgggtctcccgcttccccctcctgctccaagggcctcctgcatgagggcgcggtagagacccggacccgcgccgtgctcctgccgtttcgctgcgctccgcccgggcccggctcagccaggccccgcggtgagccatgattcgcctcggggctccccagacgctggtgctgctgacgctgctcgtcgccgctgtccttcggtgtcagggccaggatgtccaggaggctggcagctgtgtgcaggatgggcagaggtataatgataaggatgtgtggaagccggagccctgccggatctgtgtctgtgacactgggactgtcctctgcgacgacataatctgtgaagacgtgaaagactgcctcagccctgagatccccttcggagagtgctgccccatctgcccaactgacctcgccactgccagtgggcaaccaggaccaaagggacagaaaggagaacctggagacatcaaggatattgtaggacccaaaggacctcctgggcctcagggacctgcaggggaacaaggacccagaggggatcgtggtgacaaaggtgaaaaaggtgcccctggacctcgtggcagagatggagaacctgggacccctggaaatcctggcccccctggtcctcccggcccccctggtccccctggtcttggtggaaactttgctgcccagatggctggaggatttgatgaaaaggctggtggcgcccagttgggagtaatgcaaggaccaatgggccccatgggacctcgaggacctccaggccctgcaggtgctcctgggcctcaaggatttcaaggcaatcctggtgaacctggtgaacctggtgtctctggtcccatgggtccccgtggtcctcctggtccccctggaaagcctggtgatgatggtgaagctggaaaacctggaaaagctggtgaaaggggtccgcctggtcctcagggtgctcgtggtttcccaggaaccccaggccttcctggtgtcaaaggtcacagaggttatccaggcctggacggtgctaagggagaggcgggtgctcctggtgtgaagggtgagagtggttccccgggtgagaacggatctccgggcccaatgggtcctcgtggcctgcctggtgaaagaggacggactggccctgctggcgctgcgggtgcccgaggcaacgatggtcagccaggccccgcagggcctccgggtcctgtcggtcctgctggtggtcctggcttccctggtgctcctggagccaagggtgaagccggccccactggtgcccgtggtcctgaaggtgctcaaggtcctcgcggtgaacctggtactcctgggtcccctgggcctgctggtgcctccggtaaccctggaacagatggaattcctggagccaaaggatctgctggtgctcctggcattgctggtgctcctggcttccctgggccacggggccctcctggccctcaaggtgcaactggtcctctgggcccgaaaggtcagacgggtgaacctggtattgctggcttcaaaggtgaacaaggccccaagggagaacctggccctgctggcccccagggagcccctggacccgctggtgaagaaggcaagagaggtgcccgtggagagcctggtggcgttgggcccatcggtccccctggagaaagaggtgctcccggcaaccgcggtttcccaggtcaagatggtctggcaggtcccaagggagcccctggagagcgagggcccagtggtcttgctggccccaagggagccaacggtgaccctggccgtcctggagaacctggccttcctggagcccggggtctcactggccgccctggtgatgctggtcctcaaggcaaagttggcccttctggagcccctggtgaagatggtcgtcctggacctccaggtcctcagggggctcgtgggcagcctggtgtcatgggtttccctggccccaaaggtgccaacggtgagcctggcaaagctggtgagaagggactgcctggtgctcctggtctgaggggtcttcctggcaaagatggtgagacaggtgctgcaggaccccctggccctgctggacctgctggtgaacgaggcgagcagggtgctcctgggccatctgggttccagggacttcctggccctcctggtcccccaggtgaaggtggaaaaccaggtgaccagggtgttcccggtgaagctggagcccctggcctcgtgggtcccaggggtgaacgaggtttcccaggtgaacgtggctctcccggtgcccagggcctccagggtccccgtggcctccccggcactcctggcactgatggtcccaaaggtgcatctggcccagcaggcccccctggggctcagggccctccaggtcttcagggaatgcctggcgagaggggagcagctggtatcgctgggcccaaaggcgacaggggtgacgttggtgagaaaggccctgagggagcccctggaaaggatggtggacgaggcctgacaggtcccattggcccccctggcccagctggtgctaatggcgagaagggagaagttggacctcctggtcctgcaggaagtgctggtgctcgtggcgctccgggtgaacgtggagagactgggccccccggaccagcgggatttgctgggcctcctggtgctgatggccagcctggggccaagggtgagcaaggagaggccggccagaaaggcgatgctggtgcccctggtcctcagggcccctctggagcacctgggcctcagggtcctactggagtgactggtcctaaaggagcccgaggtgcccaaggccccccgggagccactggattccctggagctgctggccgcgttggacccccaggctccaatggcaaccctggaccccctggtccccctggtccttctggaaaagatggtcccaaaggtgctcgaggagacagcggcccccctggccgagctggtgaacccggcctccaaggtcctgctggaccccctggcgagaagggagagcctggagatgacggtccctctggtgccgaaggtccaccaggtccccagggtctggctggtcagagaggcatcgtcggtctgcctgggcaacgtggtgagagaggattccctggcttgcctggcccgtcgggtgagcccggcaagcagggtgctcctggagcatctggagacagaggtcctcctggccccgtgggtcctcctggcctgacgggtcctgcaggtgaacctggacgagagggaagccccggtgctgatggcccccctggcagagatggcgctgctggagtcaagggtgatcgtggtgagactggtgctgtgggagctcctggagcccctgggccccctggctcccctggccccgctggtccaactggcaagcaaggagacagaggagaagctggtgcacaaggccccatgggaccctcaggaccagctggagcccggggaatccagggtcctcaaggccccagaggtgacaaaggagaggctggagagcctggcgagagaggcctgaagggacaccgtggcttcactggtctgcagggtctgcccggccctcctggtccttctggagaccaaggtgcttctggtcctgctggtccttctggccctagaggtcctcctggccccgtcggtccctctggcaaagatggtgctaatggaatccctggccccattgggcctcctggtccccgtggacgatcaggcgaaaccggccctgctggtcctcctggaaatcctggaccccctggtcctccaggtccccctggccctggcatcgacatgtccgcctttgctggcttaggcccgagagagaagggccccgaccccctgcagtacatgcgggccgaccaggcagccggtggcctgagacagcatgacgccgaggtggatgccacactcaagtccctcaacaaccagattgagagcatccgcagccccgagggctcccgcaagaaccctgctcgcacctgcagagacctgaaactctgccaccctgagtggaagagtggagactactggattgaccccaaccaaggctgcaccttggacgccatgaaggttttctgcaacatggagactggcgagacttgcgtctaccccaatccagcaaacgttcccaagaagaactggtggagcagcaagagcaaggagaagaaacacatctggtttggagaaaccatcaatggtggcttccatttcagctatggagatgacaatctggctcccaacactgccaacgtccagatgaccttcctacgcctgctgtccacggaaggctcccagaacatcacctaccactgcaagaacagcattgcctatctggacgaagcagctggcaacctcaagaaggccctgctcatccagggctccaatgacgtggagatccgggcagagggcaatagcaggttcacgtacactgccctgaaggatggctgcacgaaacataccggtaagtggggcaagactgttatcgagtaccggtcacagaagacctcacgcctccccatcattgacattgcacccatggacataggagggcccgagcaggaattcggtgtggacatagggccggtctgcttcttgtaaaaacctgaacccagaaacaacacaatccgttgcaaacccaaaggacccaagtactttccaatctcagtcactctaggactctgcactgaatggctgacctgacctgatgtccattcatcccaccctctcacagttcggacttttctcccctctctttctaagagacctgaactgggcagactgcaaaataaaatctcggtgttctatttatttattgtcttcctgtaagaccttcgggtcaaggcagaggcaggaaactaactggtgtgagtcaaatgccccctgagtgactgcccccagcccaggccagaagacctcccttcaggtgccgggcgcaggaactgtgtgtgtcctacacaatggtgctattctgtgtcaaacacctctgtattttttaaaacatcaattgatattaaaaatgaaaagattattggaaagtaca

SEQ ID NO: 26 - DNA, 4731 bp, human
agcaacagagtttagactgtctttgcttcatcatctgaaggtaaaattttccagatacggcagacggctttcagagtacaataaacagggaatgagaactatttacatggaagtttctttctcatgatgcggtggagaagcctcggccacttggttctgccagatgttcctggggttactgtaaatgggaaggacaggcagagctaaacaaggtttatcatttaaaagtgcctgtgtgaagtcacttttgctggaaaactgcagcttgggagctttctttgtattcacatcccactcttctgtcaagtacactttaccctgaccttatgagtggatgaagatacctcagttgtctgactttgccaattgcttaatttcagaatttaaaaaggggaaagaaaaacatcctgctaaaatatgaacatctgagtgtcttattttccaacatcgtcaatagctgtgagcgtcagcattaaatattctcccaaggagtgccatgatattgaagtcactttattaataacagctgtatctgcaaaacagtcaagagactcggacgttgaaagccagagatgacactgagcatgcttttattgcggcctaccatctttaagtgggacatattgattgatgagtgattgcctgtccatacactctctcatcatcctgttccttggattggacttcactaagcaatttatcactcaccttcagacttacatgtgggagttttcacaacagtagttttggaatcattagaacttggattgatttcatcatttaacagaaacaaacagcccaaattactttatcaccatggctttgaacgttgccccagtcagagatacaaaatggctgacattagaagtctgcagacagtttcaaagaggaacatgctcacgctctgatgaagaatgcaaatttgctcatccccccaaaagttgtcaggttgaaaatggaagagtaattgcctgctttgattccctaaagggccgttgttcgagagagaactgcaagtatcttcaccctccgacacacttaaaaactcaactagaaattaatggaaggaacaatttgattcagcaaaaaactgcagcagcaatgcttgcccagcagatgcaatttatgtttccaggaacaccacttcatccagtgcccactttccctgtaggtcccgcgatagggacaaatacggctattagctttgctccttacctagcacctgtaacccctggagttgggttggtcccaacggaaattctgcccaccacgcctgttattgttcccggaagtccaccggtcactgtcccgggctcaactgcaactcagaaacttctcaggactgacaaactggaggtatgcagggagttccagcgaggaaactgtgcccggggagagaccgactgccgctttgcacaccccgcagacagcaccatgatcgacacaagtgacaacaccgtaaccgtttgtatggattacataaaggggcgttgcatgagggagaaatgcaaatattttcaccctcctgcacacttgcaggccaaaatcaaagctgcgcagcaccaagccaaccaagctgcggtggccgcccaggcagccgcggccgcggccacagtcatggcctttccccctggtgctcttcatcctttaccaaagagacaagcacttgaaaaaagcaatggtaccagcgcggtctttaaccccagcgtcttgcactaccagcaggctctcaccagcgcacagttgcagcaacacgccgcgttcattccaacagggtcagttttgtgcatgacacccgctaccagtattgtacccatgatgcacagcgctacgtccgccactgtctctgcagcaacaactcctgcaacaagtgtccccttcgcagcaacagccacagccaatcagataattctgaaataatcagcagaaacggaatggaatgccaagaatctgcattgagaataactaaacattgttactgtacatactatcctgtttcctcctcaatagaattgccacaaactgcatgctaaataaagatgtagttcttctggacagaccacaactctaagaagctagtgctgctatctcatatatgagtattaaatatggtatgcttagtatattccaacctaagatagttaactacctgagaccagctgtgatgtttaaagacataaaggataaagtttacttttaaagggtttctaaacatagtttctgtcctaggaatattgtcttatctccataactatagctgatgcagaaagtccagccagtttactcatttcgattcagaatatttcaaatttagcaataaacaattagcattagttaaaaaagaaacatattccaagggcaggttcgattctagctctaattactgtcatgtcatttacccactggatcaaagggtatgtttcacttcttgacaatataaatgctgcagcaaagatgagaggtgaagtaaaaccgatacctgtcctgcaggtctaaaatttgaatggaaattcaagcacaagtactggggacacatcaaagtgtggtgtttggtttgcctggagatgccacgttgaatcatgtgattctagattaacattaaatagattgaaaaagaaactttgcacggtatgagcttcataccccaccaaacaaagtcttgaaggtattattttacaagtatatttttaaagttgttttataagagagactttgtagaagtgcctagattttgccagacttcatccagcttgacaagattgagaggcccatgccaacagtctaatctaagagattagtctttcaaactcaccatccagttgcctgttacagaataactcttcttaactaaaaacctagtcaaacaaggaagctgtaggtgaggagatctgtataatattctaatttaagtaagtttgagtttagtcactgcaaatttgactgtgactttaatctaaattactatgtaaacaaaaagtagatagtttcactttttaaaaaatccattactgttttgcatttcaaaagttggattaaagggttgtaactgactacagcatggaaaaaaatagttcttttaattctttcaccttaaagcatattttatgtctcaaaagtataaaaaactttaatacaagtacatacatattatatatacacatacatatatatactatatatggatgaaacatattttaatgttgtttacttttttaaatacttggttgatcttcaaggtaatagcgatacaattaaattttgttcagaaagtttgttttaaagtttattttaagcactatcgtaccaaatatttcatatttcacattttatatgttgcacatagcctatacagtacctacatagtttttaaattattgtttaaaaaacaaaacagctgttataaatgaatattatgtgtaattgtttcaaacatccattttctttgtgaacatattagtgattgaagtattttgacttttgagattgaatgtaaaatattttaaatttgggatcatcgcctgttctgaaaactagatgcaccaaccgtatcattatttgtttgaggaaaaaaagaaatctgcattttaattcatgttggtcaaagtcgaattactatctatttatcttatatcgtagatctgataaccctatctaaaagaaagtcacacgctaaatgtattcttacatagtgcttgtatcgttgcatttgttttaatttgtggaaaagtattgtatctaacttgtattactttggtagtttcatctttatgtattattgatatttgtaattttctcaactataacaatgtagttacgctacaacttgcctaaaacattcaaacttgttttcttttttctgtttttttctttgttaattcatttaaactcattgaaaacatagtatacattactaaaaggtaaattatgggaatcactgaaatatttttgtagattaattgttgtaacattgtctttcttttttttcttttgtttcatgattttgatttttaaaattattagcacacaactattttcagccctttaataatggagcatcaaaaacatcacctgtaaccccaagcaaatatagaagactgtattttttactatgatatccattttccagaattgtgattacaatatgcaaagagtcataaatatgccatttacaataaggaggaggcaaggcaaatgcatagatgtacaaatatatgtacaacagattttgctttttatttatttataatgtaattttatagaataattctgggatttgagaggatctaaaactatttttctgtataaatattatttgccaaaagtttgtttatattcagaagtctgactatgatgaataaatcttaaatgctttgtttaattaaaaaacaaaaatcaccaatatccaagacatgaagatatcagttcaacaaatactgtagttaagagactaactctccacttgtatgggaactacatttcactcttggttttcaggatataacagcacttcaccgaaatattctttcagccataccactggtaacatttctactaaatctttctgtaacacttaaagaattccctcattcattaccttacagtgtaaacaggagtctaatttgtatcaatactatgttttggttgtaatattcagttcactcacccaatgtacaaccaatgaaataaaagaagcatttaaaaggaaaaaaaaaaaaaaaaaaaaaaaaaaaa

SEQ ID NO: 27 - DNA, 5680 bp, human
gggcgatgggttgatgggcgccgggggacgcaggatgcggggggcgcccgcgcgcctgctgctgccgctgctgccgtggctcctgctgctcctggcgcccgaggctcggggcgcgcccggctgcccgctatccatccgcagctgcaagtgctcgggggagcggcccaaggggctgagcggcggcgtccctggcccggctcggcggagggtggtgtgcagcggcggggacctcccggagcctcccgagcccggccttctgcctaacggcaccgttaccctgctcttgagcaataacaagatcacggggctccgcaatggctccttcctgggactgtcactgctggagaagctggacctgaggaacaacatcatcagcacagtgcagccgggcgccttcctgggcctgggggagctgaagcgtttagatctctccaacaaccggattggctgtctcacctccgagaccttccagggcctccccaggcttctccgactaaacatatctggaaacatcttctccagtctgcaacctggggtctttgatgagctgccagcccttaaggttgtggacttgggcaccgagttcctgacctgtgactgccacctgcgctggctgctgccctgggcccagaatcgctccctgcagctgtcggaacacacgctctgtgcttaccccagtgccctgcatgctcaggccctgggcagcctccaggaggcccagctctgctgcgagggggccctggagctgcacacacaccacctcatcccgtccctacgccaagtggtgttccagggggatcggctgcccttccagtgctctgccagctacctgggcaacgacacccgcatccgctggtaccacaaccgagcccctgtggagggtgatgagcaggcgggcatcctcctggccgagagcctcatccacgactgcaccttcatcaccagtgagctgacgctgtctcacatcggcgtgtgggcctcaggcgagtgggagtgcaccgtgtccatggcccaaggcaacgccagcaagaaggtggagatcgtggtgctggagacctctgcctcctactgccccgccgagcgtgttgccaacaaccgcggggacttcaggtggccccgaactctggctggcatcacagcctaccagtcctgcctgcagtatcccttcacctcagtgcccctgggcgggggtgccccgggcacccgagcctcccgccggtgtgaccgtgccggccgctgggagccaggggactactcccactgtctctacaccaacgacatcaccagggtgctgtacaccttcgtgctgatgcccatcaatgcctccaatgcgctgaccctggctcaccagctgcgcgtgtacacagccgaggccgctagcttttcagacatgatggatgtagtctatgtggctcagatgatccagaaatttttgggttatgtcgaccagatcaaagagctggtagaggtgatggtggacatggccagcaacctgatgctggtggacgagcacctgctgtggctggcccagcgcgaggacaaggcctgcagccgcatcgtgggtgccctggagcgcattgggggggccgccctcagcccccatgcccagcacatctcagtgaatgcgaggaacgtggcattggaggcctacctcatcaagccgcacagctacgtgggcctgacctgcacagccttccagaggagggagggaggggtgccgggcacacggccaggaagccctggccagaaccccccacctgagcccgagcccccagctgaccagcagctccgcttccgctgcaccaccgggaggcccaatgtttctctgtcgtccttccacatcaagaacagcgtggccctggcctccatccagctgcccccgagtctattctcatcccttccggctgccctggctcccccggtgcccccagactgcaccctgcaactgctcgtcttccgaaatggccgcctcttccacagccacagcaacacctcccgccctggagctgctgggcctggcaagaggcgtggcgtggccacccccgtcatcttcgcaggaaccagtggctgtggcgtgggaaacctgacagagccagtggccgtttcgctgcggcactgggctgagggagccgaacctgtggccgcttggtggagccaggaggggcccggggaggctgggggctggacctcggagggctgccagctccgctccagccagcccaatgtcagcgccctgcactgccagcacttgggcaatgtggccgtgctcatggagctgagcgcctttcccagggaggtggggggcgccggggcagggctgcaccccgtggtatacccctgcacggccttgctgctgctctgcctcttcgccaccatcatcacctacatcctcaaccacagctccatccgtgtgtcccggaaaggctggcacatgctgctgaacttgtgcttccacatagccatgacctctgctgtctttgcggggggcatcacactcaccaactaccagatggtctgccaggcggtgggcatcaccctgcactactcctccctatccacgctgctctggatgggcgtgaaggcgcgagtgctccataaggagctcacctggagggcaccccctccgcaagaaggggaccccgctctgcctactcccagtcctatgctccggttctatttgatcgctggagggattccactcattatctgtggcatcacagctgcagtcaacatccacaactaccgggaccacagcccctactgctggctggtgtggcgtccaagccttggcgccttctacatccctgtggctttgattctgctcatcacctggatctatttcctgtgcgccgggctacgcttacggggtcctctggcacagaaccccaaggcgggcaacagcagggcctccctggaggcaggggaggagctgaggggttccaccaggctcaggggcagcggccccctcctgagtgactcaggttcccttcttgctactgggagcgcgcgagtggggacgcccgggcccccggaggatggtgacagcctctattctccgggagtccagctaggggcgctggtgaccacgcacttcctgtacttggccatgtgggcctgcggggctctggcagtgtcccagcgctggctgccccgggtggtgtgcagctgcttgtacggggtggcagcctccgccctgggcctcttcgtcttcactcaccactgtgccaggcggagggacgtgagagcctcgtggcgcgcctgctgcccccctgcctctcccgcggccccccatgccccgccccgggccctgcccgccgccgcagaggacggttccccggtgttcggggaggggcccccctccctcaagtcctccccaagcggcagcagcggccatccgctggctctgggcccctgcaagctcaccaacctgcagctggcccagagtcaggtgtgcgaggcgggggcggcggccggcggggaaggagagccggagccggcgggcacccggggaaacctcgcccaccgccaccccaacaacgtgcaccacgggcgtcgggcgcacaagagccgggccaagggacaccgcgcgggggaggcctgcggcaagaaccggctcaaggccctgcgcgggggcgcggcgggggcgctggagctgctgtccagcgagagcggtagtctgcacaacagccccaccgacagctacctgggcagcagccgcaacagcccgggcgccggcctgcagctggaaggcgagcccatgctcacgccgtccgagggcagcgacaccagcgccgcgccgctttctgaggcgggccgggcaggccagcgccgcagcgccagccgcgacagtctcaagggcggcggcgcgctggagaaggagagccatcgccgctcgtacccgctcaacgccgccagcctaaacggcgcccccaaggggggcaagtacgacgacgtcaccctgatgggcgcggaggtagccagcggcggctgcatgaagaccggactctggaagagcgaaactaccgtctaaggtggggcgggcgacgcggtagacgggctggccacgcggctcgttcccccgctcctcggggccctccaaggtgtctccgtagtcagcaggttggaggcagaggagccgatggctggaggaagcccacaggcggatgttccccacttgcctagagggcatccctctggggtagcgacagacaatcccagaaacacgcataatacatttccgtccagcccggggcagtctgactgtcggtgccctcccaggaacggggaaggcctccgtctgtgtgaaagggcacagcacatcccaggtgcaccctccccaagtactcccaccccgcctactgtccatgcggcctcactgggggccatcagcctcaccagcaaagcagagatgagagcgtgggaactgtgttctttcctccctgccctctactgatttcagcccagcccctgcctagatcctaggtcccttttcctcccgagtttggctggcacgagagctagcccagcacatgaagcaggtgatgttaagtcacaaggtgctgcttttcagatccactatgcaagaggggagggtggggccacgtgaaaggcagctctagacatcaaccagtcctgggggaggggagtgggaaccgggcacaactaggaacaatgccaccattcccacaggagtggtacttaaaccagacagcagggttcagaggtggcacaccgggacaaagctgaggccctgcacctcaacagctgactgccaggtgcctgtgggtgaactgaggggagtagagggagagggcaggtggaactggggcagaatctagtcatgccctaaagctagtcctgtaaacaatggtgccccagaaagctgcaggtggtgtttggagaagcagttacttttcagttacaagacccatctccctagtctcagccttacaacaccacgggactaaggaagagcacttccttgcctccgtaaggccagaggaagaaccatcccaatcatttgatctccagctccacagtagagagaaacctacaaaatgtcaaaccagcttcccgactcccaggagctcaagccaagcccagaggcagtggctggggtccctgcaggtcatgaggggcctatgcctttactccttttaaacaccagcacccgtcttttccccaacctaaaaccaaccaccagcatttcactacaggaccaaatggaaaccgagggaaccctgggtcttgggaagaacaacaggaaaccaaggtctgacctagggttccctcccagtcttcacatcactctggcctcatcaccaaggtgacagaggacacaggggagggggaaaacccacacacactccttggaatgggtcctgttatttatgcttgctgcacagacatattagaagaaaaaaaaaagctttgtattattcttccacatatgctggctgctgtttacacaccctgccaatgccttagcactggagagctttttgcaatatgctggggaaaggggagggagggaatgaaagtgccaaagaaaacatgtttttaagaactcgggttttatacaatagaatgttttctagcagatgcctcttgttttaatatattaaaattttgcaaagccctttgagctacaaaaaaaaaaaaaaaaaaaaaaaaaaaaaaa

SEQ ID NO: 28 - DNA, 2775 bp, human
gcagtccagatgtcgtcagcaccagcgcctgggctggaggacagagaagccttttccgttgccggtgccggcctagcgtcctggaattacttcaatcaacaggagcgagaacccgagcagcgccatgagcaacactaccgtcgtccccagcactgcaggtccgggccccagcggcgggcccggtggcggaggtggtggtggcggcggaggcggcggcaccgaggtaatccaggtgactaatgtctccccgagcgctagctctgagcagatgcggactctcttcggtttcctaggcaagatcgacgaactgcgcctcttcccgccggatgattcgcctttgccagtctcatctcgtgtctgctttgttaagttccatgatccagactcagcagttgtggcacagcatctgacaaacactgtattcgttgacagagctttgatagtcgtaccatatgcagaaggagttattcctgatgaagctaaagctttgtctctgttggcaccagctaatgcagtggcaggtcttctgcctggtggtggactcctgcctactcctaacccacttacccagattggcgctgttccactggctgctttgggggctcctactcttgatcctgcccttgctgcacttgggcttcctggagcaaacttgaactctcagtctcttgctgcagatcagttgctgaagcttatgagtactgttgatcccaagttgaatcatgtagctgctggtctcgtttcaccaagtctgaaatcggatacctctagtaaagaaatagaggaagctatgaaaagagtacgagaagcacagtccctaatttctgctgctatagaaccagataagaaagaagaaaaaagaaggcattcaagatcaagatcacgttctaggaggaggaggactccctcatcttctagacacaggcggtcaagaagcagatcgagacggcggtcacattctaagtctaggagtcggcgacgatccaaaagcccaaggcggagaagatctcattccagagaaagaggtagaaggtcaaggagcacatcaaaaacaagagacaaaaagaaagaagacaaagaaaagaaacgttctaaaacaccaccaaaaagttacagcacagccagacgttctagaagtgcaagcagagagagacgacgacgaagaagcaggagtggcacaagatctcctaaaaagcctcggtctcctaaaagaaaattgtcccgctcaccatcccctaggagacataaaaaggagaagaagaaagataaagacaaagaaagaagtagggatgaaagagaacgatcaacaagcaagaagaagaagagtaaagataaggaaaaggaccgggaaagaaaatcagagagtgataaagatgtaaaacaggttacacgggattatgatgaagaggaacaggggtatgacagtgagaaagagaaaaaagaagagaagaaaccaatagaaacaggttcccctaaaacaaaggaatgttctgtggaaaagggaactggtgattcactaagagaatccaaagtgaatggggatgatcatcatgaagaagacatggatatgagtgactgaatattgcctctgagggagtccaactgtatacctgcatcagtgtcattcctttgtgtgatttcttaatgctgtatttgttcatctcaaacctagatgtatacagctctgagttataaatggttataaagctcctgttactcatattagttatttacatcaaaaagcttttagaaaatggtacgaggtaaccaattcttgtcatggtgaaatctgattgagtaaccaagcagttttactattctggtgctgcttcataacaaaaatgaaaagctgcatgcatctacagcaggcatggattgtttatgtcgtatgatatcctttattaagtaagttcacttatagtatttctataatttgattcattgccgtaatagagccatgtaggaaatgcactgattgcatgttattgtggcaagaatatcctaaatgtcattaaaatcctccaacatgatggatctacttatggtcttgtttgttgacatgacaaattaacattcttatagttacatctggaaatgagcatttgaaatagataatcctttaagccttgtggcaaaatttttgtggcttttgtttaactttgaaaggttattatgcactaaccttttttggtggctaattagggtttaaatacagaaacaagatttcaaataaaactgtctttggcagtgagtaaatagcatattttgaagtagagttgtatactttttcataagatgtttgggaatttttttcctgaagtaataatttattccacatctacatcagtgaaagctatctacctatcctgagtctatcttaaaggaaaaaaagaaaaaaaccttatctcttgcccttattttgaattttccactctttcattaatttgttttaagctccgtgttggaaaaaaggggtagtgcattttaaattgaccttcatacgcttttaaaataagacaaatctacttgataatgtacctttatttgatctcaagttgtataaaaccaataaatttgtgttactgcagtagtaatcttatgcacacggtgatttcatgttatatatgcaaagtaggcaactgttttcttagttacagaagtttcaagcttcacttttgtgcagtagaaacaaaagtaggctacagtctgtgccatgttgatgtacagtttctgaaattgttttacaagactttgataataaaacccttaaactta

SEQ ID NO: 29 - DNA, 1785 bp, human
gagagggcgaaggtaggctggcagatacgttcgtcagcttgctcctttctgcccgtggacgccgccgaagaagcatcgttaaagtctctcttcaccctgccgtcatgtctaagtcagagtctcctaaagagcccgaacagctgaggaagctcttcattggagggttgagctttgaaacaactgatgagagcctgaggagccattttgagcaatggggaacgctcacggactgtgtggtaatgagagatccaaacaccaagcgctccaggggctttgggtttgtcacatatgccactgtggaggaggtggatgcagctatgaatgcaaggccacacaaggtggatggaagagttgtggaaccaaagagagctgtctccagagaagattctcaaagaccaggtgcccacttaactgtgaaaaagatatttgttggtggcattaaagaagacactgaagaacatcacctaagagattattttgaacagtatggaaaaattgaagtgattgaaatcatgactgaccgaggcagtggcaagaaaaggggctttgcctttgtaacctttgacgaccatgactccgtggataagattgtcattcagaaataccatactgtgaatggccacaactgtgaagttagaaaagccctgtcaaagcaagagatggctagtgcttcatccagccaaagaggtcgaagtggttctggaaactttggtggtggtcgtggaggtggtttcggtgggaatgacaacttcggtcgtggaggaaacttcagtggtcgtggtggctttggtggcagccgtggtggtggtggatatggtggcagtggggatggctataatggatttggtaatgatggaagcaattttggaggtggtggaagctacaatgattttgggaattacaacaatcagtcttcaaattttggacccatgaagggaggaaattttggaggcagaagctctggcccctatggcggtggaggccaatactttgcaaaaccacgaaaccaaggtggctatggcggttccagcagcagcagtagctatggcagtggcagaagattttaattaggaaacaaagcttagcaggagaggagagccagagaagtgacagggaagctacaggttacaacagatttgtgaactcagccaagcacagtggtggcagggcctagctgctacaaagaagacatgttttagacaaatactcatgtgtatgggcaaaaaactcgaggactgtatttgtgactaattgtataacaggttattttagtttctgttctgtggaaagtgtaaagcattccaacaaagggttttaatgtagatttttttttttgcaccccatgctgttgattgctaaatgtaacagtctgatcgtgacgctgaataaatgtcttttttttaatgtgctgtgtaaagttagtctactcttaagccatcttggtaaatttccccaacagtgtgaagttagaattccttcagggtgatgccaggttctatttggaatttatatacaacctgcttgggtggagaagccattgtcttcggaaaccttggtgtagttgaactgatagttactgttgtgacctgaagttcaccattaaaagggattacccaagcaaaatcatggaatggttataaaagtgattgttggcacatcctatgcaatatatctaaattgaataatggtaccagataaaattatagatgggaatgaagcttgtgtatccattatcatgtgtaatcaataaacgatttaattctcttgaaaaaaaaaaaaaaaaaa

SEQ ID NO: 30 - DNA, 10284 bp, human
gcggcccggtgcgggtgtcggggagaccgggctctctgcccggcgcggcgcggcgcggctcggcccacgagcgaccaccgacatggagtgggctcgggcggccaagtagccgcttctccggagcccggtgccagtgccgcccgcagcccgccttccacccccggccgcgccgccggtcaggccctagggtgaagccgggaggaaaatgaagagttttcaccggaatccgttgaaaataggactgactgcaaagccttaaagaaagaaggacctcgggaggagaaacgaaaagccgcctccgggcaagacttggcgtgctccgagccgaggggctgcttcagggacctcgccccctccctttcccgctggagaaattgccgctgatgcattatccaagtggtggttgggaggatttgcagcaacatttttggttttccctcccccttctatgcattctgtttttttcctcccttttctgtttttcttcttcccgggaagtgaattgctgatgcaaatcggactttattcattaatgatgcaaccggattcgtttcaggattacgttgcacgagttgaattttgaatgaaggagaagagtttttttttttttttttaaagaagtgttgactctctagttcgttgtacttttaattattattttatttaaatatacgacttaattgtattcttttaaaaatgcattaagtatatattttatggtaatttaccctcaaaatatatgtatatgggtgaaattgaagacgcttcagttaagtgaggttactggtgtgttggatgtttaattcagcaccagcattgcatgacagttgtttgaataacaagtggtttatttttaaaaccataccttttaaaatttaggttcagataatagtaaaagtcatcataataatttaaaggaaaaccagcagaaatcgaagcaaacatgtctggagaagtgcgtttgaggcagttggagcagtttattttggacgggcccgctcagaccaatgggcagtgcttcagtgtggagacattactggatatactcatctgcctttatgatgaatgcaataattctccattgagaagagagaagaacattctcgaatacctagaatgggctaaaccatttacttctaaagtgaaacaaatgcgattacatagagaagactttgaaatattaaaggtgattggtcgaggagcttttggggaggttgctgtagtaaaactaaaaaatgcagataaagtgtttgccatgaaaatattgaataaatgggaaatgctgaaaagagctgagacagcatgttttcgtgaagaaagggatgtattagtgaatggagacaataaatggattacaaccttgcactatgctttccaggatgacaataacttatacctggttatggattattatgttggtggggatttgcttactctactcagcaaatttgaagatagattgcctgaagatatggctagattttacttggctgagatggtgatagcaattgactcagttcatcagctacattatgtacacagagacattaaacctgacaatatactgatggatatgaatggacatattcggttagcagattttggttcttgtctgaagctgatggaagatggaacggttcagtcctcagtggctgtaggaactccagattatatctctcctgaaatccttcaagccatggaagatggaaaagggagatatggacctgaatgtgactggtggtctttgggggtctgtatgtatgaaatgctttacggagaaacaccattttatgcagaatcgctggtggagacatacggaaaaatcatgaaccacaaagagaggtttcagtttccagcccaagtgactgatgtgtctgaaaatgctaaggatcttattcgaaggctcatttgtagcagagaacatcgacttggtcaaaatggaatagaagactttaagaaacacccatttttcagtggaattgattgggataatattcggaactgtgaagcaccttatattccagaagttagtagcccaacagatacatcgaattttgatgtagatgatgattgtttaaaaaattctgaaacgatgcccccaccaacacatactgcattttctggccaccatctgccatttgttggttttacatatactagtagctgtgtactttctgatcggagctgtttaagagttacggctggtcccacctcactggatcttgatgttaatgttcagaggactctagacaacaacttagcaactgaagcttatgaaagaagaattaagcgccttgagcaagaaaaacttgaactcagtagaaaacttcaagagtcaacacagactgtccaagctctgcagtattcaactgttgatggtccactaacagcaagcaaagatttagaaataaaaaacttaaaagaagaaattgaaaaactaagaaaacaagtaacagaatcaagtcatttggaacagcaacttgaagaagctaatgctgtgaggcaagaactagatgatgcttttagacaaatcaaggcttatgaaaaacaaatcaaaacgttacaacaagaaagagaagatctaaataagctggaagttcatacagaagctctagctgctgaagcatctaaagacaggaagctacgtgaacagagtgagcactattctaagcaactggaaaatgaattggagggactgaagcaaaaacaaattagttactcaccaggagtatgcagcatagaacatcagcaagagataaccaaactaaagactgatttggaaaagaaaagtatcttttatgaagaagaattatctaaaagagaaggaatacatgcaaatgaaataaaaaatcttaagaaagaactgcatgattcagaaggtcagcaacttgctctcaacaaagaaattatgattttaaaagacaaattggaaaaaaccagaagagaaagtcaaagtgaaagggaggaatttgaaagtgagttcaaacaacaatatgaacgagaaaaagtgttgttaactgaagaaaataaaaagctgacgagtgaacttgataagcttactactttgtatgagaacttaagtatacacaaccagcagttagaagaagaggttaaagatctagcagacaagaaagaatcagttgcacattgggaagcccaaatcacagaaataattcagtgggtcagcgatgaaaaggatgcacgagggtatcttcaggccttagcttctaaaatgactgaagaattggaggcattaagaaattccagcttgggtacacgagcaacagatatgccctggaaaatgcgtcgttttgcgaaactggatatgtcagctagactggagttgcagtcggctctggatgcagaaataagagccaaacaggccatccaagaagagttgaataaagttaaagcatctaatatcataacagaatgtaaactaaaagattcagagaagaagaacttggaactactctcagaaatcgaacagctgataaaggacactgaagagcttagatctgaaaagggtatagagcaccaagactcacagcattctttcttggcatttttgaatacgcctaccgatgctctggatcaatttgaaactgtagactccactccactttcagttcacacaccaaccttaaggaaaaaaggatgtcctggttcaactggctttccacctaagcgcaagactcaccagttttttgtaaaatcttttactactcctaccaagtgtcatcagtgtacctccttgatggtgggtttaataagacagggctgttcatgtgaagtgtgtggattctcatgccatataacttgtgtaaacaaagctccaaccacttgtccagttcctcctgaacagacaaaaggtcccctgggtatagatcctcagaaaggaataggaacagcatatgaaggtcatgtcaggattcctaagccagctggagtgaagaaagggtggcagagagcactggctatagtgtgtgacttcaaactctttctgtacgatattgctgaaggaaaagcatctcagcccagtgttgtcattagtcaagtgattgacatgagggatgaagaattttctgtgagttcagtcttggcttctgatgttatccatgcaagtcggaaagatataccctgtatatttagggtcacagcttcccagctctcagcatctaataacaaatgttcaatcctgatgctagcagacactgagaatgagaagaataagtgggtgggagtgctgagtgaattgcacaagattttgaagaaaaacaaattcagagaccgctcagtctatgttcccaaagaggcttatgacagcactctacccctcattaaaacaacccaggcagccgcaatcatagatcatgaaagaattgctttgggaaacgaagaagggttatttgttgtacatgtcaccaaagatgaaattattagagttggtgacaataagaagattcatcagattgaactcattccaaatgatcagcttgttgctgtgatctcaggacgaaatcgtcatgtacgactttttcctatgtcagcattggatgggcgagagaccgatttttacaagctgtcagaaactaaagggtgtcaaaccgtaacttctggaaaggtgcgccatggagctctcacatgcctgtgtgtggctatgaaaaggcaggtcctctgttatgaactatttcagagcaagacccgtcacagaaaatttaaagaaattcaagtcccatataatgtccagtggatggcaatcttcagtgaacaactctgtgtgggattccagtcaggatttctaagataccccttgaatggagaaggaaatccatacagtatgctccattcaaatgaccatacactatcatttattgcacatcaaccaatggatgctatctgcgcagttgagatctccagtaaagaatatctgctgtgttttaacagcattgggatatacactgactgccagggccgaagatctagacaacaggaattgatgtggccagcaaatccttcctcttgttgttacaatgcaccatatctctcggtgtacagtgaaaatgcagttgatatctttgatgtgaactccatggaatggattcagactcttcctctcaaaaaggttcgacccttaaacaatgaaggatcattaaatcttttagggttggagaccattagattaatatatttcaaaaataagatggcagaaggggacgaactggtagtacctgaaacatcagataatagtcggaaacaaatggttagaaacattaacaataagcggcgttattccttcagagtcccagaagaggaaaggatgcagcagaggagggaaatgctacgagatccagaaatgagaaataaattaatttctaatccaactaattttaatcacatagcacacatgggtcctggagatggaatacagatcctgaaagatctgcccatgaaccctcggcctcaggaaagtcggacagtattcagtggctcagtcagtattccatctatcaccaaatcccgccctgagccaggccgctccatgagtgctagcagtggcttgtcagcaaggtcatccgcacagaatggcagcgcattaaagagggaattctctggaggaagctacagtgccaagcggcagcccatgccctccccgtcagagggctctttgtcctctggaggcatggaccaaggaagtgatgccccagcgagggactttgacggagaggactctgactctccgaggcattccacagcttccaacagttccaacctaagcagccccccaagcccagcttcaccccgaaaaaccaagagcctctccctggagagcactgaccgcgggagctgggacccgtgagctgcctcagcactgggacctctcgctctccgctccctgccactcgcctcctctcactttcatctcttccctccacctcgcctgctcggcctgaaagccaccaggggctggcagcagtagcaggacagggattcaggagttctgacgacacgactctcagatccacgcccccagcctaacagcaacaacaaagacagactttccgtagcagcttagattaacgttgatttcattccatgcacttagagttgctttcagtaacattttacccctactcccaaaggtagcttaaatagacagattacacaaatgtaagtgataagaataagattagacagattttgctttcacagtagagtctcattatagtcctaaaatagctcatgggcttctccgcatccagaagggagaattggtccctggagtggctcactaagctcttaatcagcaaacgcagtgagtatcaacctgattgttgccaggaaatccttatgaattaaaacaatgcatattttactacagtacagagtttaaatgaatacataaatgtagaagtactgaatgtatatatttaaaaggagcctcttgtattcaacaaaagatggatgcatatataagagagatgatttaatttaaagaaatatgttgtttcttgtctgtaatgtaatgtaaagggtggaaaggcctcaagctcacatttgtagagagagagcgagagaaatcagagttccctttattgccctgtcctcaaactggtcataggctctagtcacctggggagctgtagaaaacacttgcagagccaggttttgctggtttggggcatgccctgggcaccagagctttaacatttgaagccacttcagcagcagcagcaaaaggcgaactcatctctacccaagatgtttcttttcctagtggtggaatttgaacacttctcactttttattgtattttatcttccgcagataaatgtagaaatacacgattctgtcacctctgatcccttccatctgaaaggttacaaggagtgttgtagcttctgaaggtgcagaaaacaatttctaaaaatgcttttattcctgggctaatcctgtccctccctaagtcacagcgaggtgtctgtcccagggctggagatgcttcccaaggaggagtctgttttgttgagagtgggcgtgggcttcttcacataagcctggggaaggaagaaaaaacggctttcattaccaaataatgtaaaacctcaaaagcaagggcttcaacagccttaaccaaatattattccccatagccagtggaaaatggatgtgacaaccccagtgcgcaggccagagtgagtgagcccagcacggcgctccgactggcttcctctctcaggtgctggattgtggggttagtggcatttccagctggattcctcctgttgtagttgccataaggaaatgagatgcagaatcagaaggatctatttctacagaatcatttcaccagttaagcacatgagtagagaaagagataaaaataaaagtatctcatgaaggaaagagattttgcctctcttttacttttcacctaagtttctctgagaaatagagacaggattctctctttaaaattcagtgaaaatgaagaaagttttcctgcagttgctaacctgagttgcagtgtttaaggccatcatttcactgctgctgtctgtgactccacgtctgtgtcactgaggtgacctgcgtgtcactgaggtggccaccatgctggcctgcggcatgtgcagggagctgaggctgtttccaggtgatgctgctgtgtggagaaggttctgagatgcagtgagggaagaaaggatcctgctggggattccattgtaagcacctataatcgggaattttcatgtaacagctttgacatttaaacattctgagtttggtgccagctcagatttgattatattttattttggatgggtgtaattcacagcacagttctaatctcccaaatctttctgctttttagaatgaagtataaaaatacttttctcacctgaataccaagggttggccctttagttggatcattgtcatatgacttggtagatccttgtcctcagcacctcacgtgagagaagggagtcagccagccggccccctgcttggtgctcgtgaccagctcgcaccccttctgtccacccttctctcctctcctccccactctccccaccctcctcactctccccaccctcctcactctccccaccctcccctcctctcctcctcactcttcccaccctccccatccccaccctccccatcctcctcttccctttccccttgccttctcctctctcccttctcttctcaggcagggaggaggccatcccaagccgagattaacaggacttgacataagccattagtttgtagctttgacaagtaattatgaatttttgttgcttataggtgcttattttgcaaaggatgcttttaagatcaaaataataaccctacctaaagtctagctccactgctatgggtcatactcttcagcctcccaacagggcagagagagagagctactgaggcttgtctaggttgccaggctaactgggcgacttgtccatattcaccccatggattgcaccatggcactctttgatttttccactgcaatggcaagtaatctcatcagtcataatagagcagtcccgaatgcgtgcagattctaaaagcagggctttaggagagaaacactgccagggggaatagttttggggagggttttcccaaaataacggtcatcctactgggtttatcccacccttaaatatgaagcctgttacctccagaagcttctgagaagaatgatgtgaaaagacagggagtgggttctaggcaaagaaaacataatgaccattcagaggagtcagtagcacagctcacagataaagtattttattactatctgaagttttcttttgttttcatgcaggacattttaaaaacgtatatggcagcagaaacctgtttctcaatagaaaaaatacattcagaggcatttctgggatagtctatctgtgttagtatttggtgctatctatgtccagccaagttatctaccctcaaattctgactaatcatgtttgtgctttgggtatttaaaattacatacatatatattctttttgccaaaaacaaaagtcttgcttcttgtcaaatgattgctaaagtagatcttacattttttgttattatgtatgtatttatacacatccccaacacacttagtgatttctgttatttcctagggagcacagctttaaggctatgagatacaactaaaaggagcccatctatttggttttccagccaattattgtactcacatttcaggggagaatctgaaattcctgtcatgtttacagcaacaatctatcattcctggctagctctcagcctctctctccttccataggttagaattatgtcattttgttacttagtggccacgtctatttctgagaaagactggttacatttatgtggcatctcaggtatcattaaggaaaagccagagcaggggtgagcagaggtcaaaaccacagacgcagcagggccatttgccgcctttggccgggatcacaaccactgcagtctcccagcaggtaggccttgccaagcctaaggctccccatccaatctagacagaggggcgctcagagcagactttgccgtagcccatgtctggtgagcacaacagggaatgaattgggcactccactcccccgtctctctggcccagccctgaactagatgagctgcatttcatggagcccattttaaaatctctttccttatgactttgttactcaagtccagagttctctgtgcacttctgctagataaggagtgtaagccctgccccccagcactggcagcacgctgggccctccccacacaggacaccgtgcagttccgggggaagctgactcaaatcaaccttgaaatctcatgaaaacaaaatgacttgtctttttatttgatagtgtaatatcattcattttataaattttttagggtttttctcgtaatattgtacagttttgcatggcctggtgtgatcattttttggttagaatataatgctgacaaatgtggatggaggggaagatactgctttagcctatcactccttattttattttgtttggttttatgccctcagtgtcttagggaactttttaagagatcctctgctaccaaacaatgatgtggattcttttgcacagaaatatttaaggtgggatggtaaaaaatgtcacaaaagactcctcaccaatactttatgttgatatcacttaatattaaccagactttgctgtattgcaataaaacagagaactgttaaaaaaaaaaaaaaaaaaaaaa

SEQ ID NO: 31 - DNA, 3140 bp, human
acttaacattgctgatgcgtcagtcttttcttcctcatctctttcaggggctggagaggcagagggagacagaggagctggtactgcagagcggtcgtctgattggctggacggtcgtagctgggctataaaagagacccctacaggcttagcaggaagacgctcagaggattctgacaatatctttaccggagaagaggcaaagtacgctcaaagccgaagccacagctcctcctgccgcatttctttcctgcttgcgaattccaagctgttaaataagatgtgcaaagggcttgcaggtctgccggcttcttgcttgaggagtgcaaaagatatgaaacatcggctaggtttcctgctgcaaaaatctgattcctgtgaacacaattcttcccacaacaagaaggacaaagtggttatttgccagagagtgagccaagaggaagtcaagaaatgggctgaatcactggaaaacctgattagtcatgaatgtgggctggcagctttcaaagctttcttgaagtctgaatatagtgaggagaatattgacttctggatcagctgtgaagagtacaagaaaatcaaatcaccatctaaactaagtcccaaggccaaaaagatctataatgaattcatctcagtccaggcaaccaaagaggtgaacctggattcttgcaccagggaagagacaagccggaacatgctagagcctacaataacctgctttgatgaggcccagaagaagattttcaacctgatggagaaggattcctaccgccgcttcctcaagtctcgattctatcttgatttggtcaacccgtccagctgtggggcagaaaagcagaaaggagccaagagttcagcagactgtgcttccctggtccctcagtgtgcctaattctcacctgaaggcagagggatgaaatgccaagactctatgctctggaaaacctgaggccaaatattgatctgtattaagctccagtgctttatccacattgtagcctaatattcatgctgcctgccatgtgtgagtcacttctacgcataaactagatatagcttttggtgtttgagtgttcatcagggtgggaccccattccagtccaattttcctaagtttctttgagggttccatgggagcaaatatctaaataatggcctggtaggtctggattttcaaagattgttggcagtttcctcctcccaacagttttacctcgggatggttggttagtgcatgtcacatgacatccacatgcacatgtattctgttggccagcacgttctccagactctagatgtttagatgaggttgagctatgatatgtgcttgtgtgtatgtctatgtgtatatattatatatacattagacacacatatacattatttctgtatatagatgtctgtgtatacatatgtatgtgtgagtgtatgtatacacacacacacacacacacacacacacttttgcaagagtgatgggaaagaccctaggtgctcataactagagtatgtgtatgtacttacatgggtgttttgatctctgttctttcatactacatttgaacagggcaaaatgaactaactgccatgtaggctaagaaagaaatgctaacctgtggaaagttggttttgtaaaattccatggatcttgctggagaagcatccaaggaacttcatgcttgatttgaccactgacagcctccaccttgagcactattctaaggagcaaataccttagctcccttgagctggttttctctgatggcacttttgagctcctaagctgccagccttcccttcttttcctgggtgctcagggcatgcttattagcagctgggttggtatggagttggcagacaggatgttcaacttaatgaagaaatacagctaaggccttgccagcaacacctgccgtaagttactggctgagtgagggcatagaagttaaaggttactgtttttatcctctatccttttttcctttcctgatcaaggtgctcttctcattttttcctgagaaccttagccatcagatgaggctccttagtttattgtggttggttgttttttctttataatggctctgggctatatgcctatatttataaaccagcagcaggggaaagattatattttataagagggaacaaattttcacaatttgaaaagcccacataagttttctcttttaaggtagaatcttgttaatttcattccaaacatcggggctaacagagactggaggcatttctttttaggctctgagactaaatgagaggaaaagaaaagaaaaaaaaaatgattgtctaaccaattgtgagaattactgtttgaaacttttcaaggcacattgaaatacttgaaaacttctcatttatgttatttatgatgttattttgtacgtgttattattattatattgttttataaatggaggtacaggatatcacctgaattattaatgaatgcccaggaagtaattttcttctcattcttctaaaactactgcctttcaaagtgcacacacacgcgtccacatacactgcattcgttgctccagtataaattacatgcatgagcacctttctggcttttaagccaatataatgggctgcaaaatgaagacaccagagtgtatgcatacaaatctcactgtattaaagatgcaggttttctaattgtacccttcttgtctctctggcaatcttgcccttaatatccctggagttcctcatcagtgtcattttctgttatacacagttccacaattttgtctctagttgacttcaaatgtgtaactttattggtcttgccctattataattgtcatgactttcagattgtatctgaactcacagactgctgtcttactaataggtctggaaggtcacgctgaatgagaagtaaattattttatgtaatacatttttgagtgtgtttttcagttgtatttccctgttatttcatcactatttccaatggtgagcttgcctgctcatgctccctggacagaatactccttccttttgcatgcctgtttctatcatgtgcttgataggcctcaaagctaatgcttccagtgaaacacacgcatcttaataataagggtaaataaacgctccatatgaaacta

SEQ ID NO: 32 - DNA, 4077 bp, human
cgggcctcgagccgaggcagcagtgggaacgactcatcctttttccagccctggggcgtggctggggtcggggtcggggtcggggccggtgggggccccgcccccgtctcctggcctgcccccttcatgggccgcgatgatggcggccctgtacccgagcacggacctctcgggcgcctcctcctcctccctgccttcctctccatcctcttcctcgccgaacgaggtgatggcgctgaaggatgtgcgggaggtgaaggaggagaatacgctgaatgagaagcttttcttgctggcgtgcgacaagggtgactattatatggttaaaaagattttggaggaaaacagttcaggtgacttgaacataaattgcgtagatgtgcttgggagaaatgctgttaccataactattgaaaacgaaaacttggatatactgcagcttcttttggactacggttgtcagaaactaatggaacgaattcagaatcctgagtattcaacaactatggatgttgcacctgtcattttagctgctcatcgtaacaactatgaaattcttacaatgctcttaaaacaggatgtatctctacccaagccccatgcagttggctgtgaatgcacattgtgttctgcaaaaaacaaaaaggatagcctccggcattccaggtttcgtcttgatatatatcgatgtttggccagtccagctctaataatgttaacagaggaggatccaattctgagagcatttgaacttagtgctgatttaaaagaactaagtcttgtggaggtggaattcaggaatgattatgaggaactagcccggcaatgtaaaatgtttgctaaggatttacttgcacaagcccggaattctcgtgaattggaagttattctaaaccatacgtctagtgacgagcctcttgacaaacggggattattagaagaaagaatgaatttaagtcgtctaaaacttgctatcaaatataaccagaaagagtttgtctcccagtctaactgccagcagttcctgaacactgtttggtttggacagatgtcgggttaccgacgcaagcccacctgtaagaagataatgactgttttgacagtaggcatcttttggccagttttgtcactttgttatttgatagctcccaaatctcagtttggcagaatcattcacacaccttttatgaaatttatcattcatggagcatcatatttcacatttctgctgttgcttaatctatactctcttgtctacaatgaggataagaaaaacacaatggggccagcccttgaaagaatagactatcttcttattctgtggattattgggatgatttggtcagacattaaaagactctggtatgaagggttggaagactttttagaagaatctcgtaatcaactcagttttgtcatgaattctctttatttggcaacctttgccctcaaagtggttgctcacaacaagtttcatgattttgctgatcggaaggattgggatgcattccatcctacactggtggcagaagggctttttgcatttgcaaatgttctaagttatcttcgtctcttttttatgtatacaaccagctctatcttgggtccattacagatttcaatgggacagatgttacaagattttggaaaatttcttgggatgtttcttcttgttttgttttctttcacaattggactgacacaactgtatgataaaggatatacttcaaaggagcagaaggactgtgtaggcatcttctgtgaacagcaaagcaatgataccttccattcgttcattggcacctgctttgctttgttctggtatattttctccttagcgcatgtggcaatctttgtcacaagatttagctatggagaagaactgcagtcctttgtgggagctgtcattgttggtacatacaatgtcgtggttgtgattgtgcttaccaaactgctggtggcaatgcttcataaaagctttcagttgatagcaaatcatgaagacaaagaatggaagtttgctcgagcaaaattatggcttagctactttgatgacaaatgtacgttacctccacctttcaacatcattccctcaccaaagactatctgctatatgattagtagcctcagtaagtggatttgctctcatacatcaaaaggcaaggtcaaacggcaaaacagtttaaaggaatggaggaatttgaaacagaagagagatgaaaactatcaaaaagtgatgtgctgcctagtgcatcgttacttgacttccatgagacagaagatgcaaagtacagatcaggcaactgtggaaaatctaaacgaactgcgccaagatctgtcaaaattccgaaatgaaataagggatttacttggctttcggacttctaaatatgctatgttttatccaagaaattaaccattttctaaatcatggagcgaataattttcaataacagatccaaaagactatattgcataacttgcaatgaaattaatgagatatatattgaaataaagaattatgtaaaagccattctttaaaatatttatagcataaatatatgttatgtaaagtgtgtatatagaattagttttttaaaccttctgttagtggctttttgcagaagcaaaacagattaagtagatagattttgttagcatgctgcttggttttcttacttagtgctttaaaatgtttttttttatgtttaagaggggcagttataaatggacacattgcccagaatgttttgtaaaatgaagaccagcaaatgtaggctgatctccttcacaggatacacttgaaatatagaagttatgttttaaatatctctgttttaggagttcacatatagttcagcatttattgtttaggagtataattttattttatctaaaataatagtctattttttcttttgtattttgttataatcttaagcaacaaagaaaaaaccctaatatttgaatctatttatgtctttcaatttaaattcacttcagtttttgttattgtaatatatttacttttacatggttataatcactttatatttttaatgtttttttcacttaatattttatatatacatttccatgtattgatgtagttagtccacatttaaatttttatagaattatatagtttttgaaaaatacagtcagtagatgttttattttttagctattcagttatgtttataagtttgcatagctacttctcgacatttggtttgttttaatttttttgtatcataatagtcctatttttttttcaagttggagtgaatgtttttagttttaagatagataggagacacttttttatcacatgtagtcacaacctgttttgtttttgtaaaacataggaagtctctttaatgcaatgatttgttttatatttggactaaggttcttgagcttatctcccaaggtactttccataatttaacacagcttctataaaagtgacttcatgcttacttgtggatcattcttgctgcttaagatgaaaagcattggttttttaaaattagagaataaaatatgtatttaaatttttggtgtgttcacataaagggatgtagctaaaatgttttcataggctattatatattctcgcagcatttccagttaagaggatattaggtatataattctcttcttaaccgaatgtcagatggtcttacgccacagggtgcaggtaacccttggtctgtaagcaccaccgatccagggatcattgtctaaataggttactattgtttgtttcatcttgcttttgcatttttattttttaatttccaaattttaagtgttccctctttggggcaaattcttataaaaatgtttattgtaaagttatatattttgtctacgatgggattatgcacttcccaattgggattttacatctggatttttagtcattctaaaaaacacctaattattaaaacatttatagagtgcctactgtatgcatgagttgagttgcttctgaggtacattttgaatgacagcatattgtaagaaaaaaaaaggtgaataaaatttgacattagattataaaaaaaaaaa

SEQ ID NO: 33 - DNA, 3947 bp, human
tttagacacaagcgagaggatcatggcggatggccccaggtgtaagcgcagaaagcaggcgaacccgcggcgcaataacgttacaaattataatactgtggtagaaacaaattcagattcagatgatgaagacaaactgcatattgtggaagaagaaagtgttacagatgcagctgactgtgaaggtgtaccagaggatgacctgccaacagaccagacagtgttaccagggaggagcagtgaaagagaagggaatgctaagaactgctgggaggatgacagaaaggaagggcaagaaatcctggggcctgaagctcaggcagatgaagcaggatgtacagtaaaagatgatgaatgcgagtcagatgcagaaaatgagcaaaaccatgatcctaatgttgaagagtttctacaacaacaagacactgctgtcatttttcctgaggcacctgaagaggaccagaggcagggcacaccagaagccagtggtcatgatgaaaatggaacaccagatgcattttcacaattactcacctgtccatattgtgatagaggctataaacgctttacctctctgaaagaacacattaaatatcgtcatgaaaagaatgaagataactttagttgctccctgtgcagttacacctttgcatacagaacccaacttgaacgtcacatgacatcacataaatcaggaagagatcaaagacatgtgacgcagtctgggtgtaatcgtaaattcaaatgcactgagtgtggaaaagctttcaaatacaaacatcacctaaaagagcacttaagaattcacagtggagagaagccatatgaatgcccaaactgcaagaaacgcttttcccattctggctcctatagctcacacataagcagtaagaaatgtatcagcttgatacctgtgaatgggcgaccaagaacaggactcaagacatctcagtgttcttcaccgtctctttcagcatcaccaggcagtcccacacgaccacagatacggcaaaagatagagaataaaccccttcaagaacaactttctgttaaccaaattaaaactgaacctgtggattatgaattcaaacccatagtggttgcttcaggaatcaactgttcaacccctttacaaaatggggttttcactggtggtggcccattacaggcaaccagttctcctcagggcatggtgcaagctgttgttctgccaacagttggtttggtgtctcccataagtatcaatttaagtgatattcagaatgtacttaaagtggcgatagatggtaatgtaataaggcaagtgttggagaataatcaagccaatcttgcatccaaagaacaagaaacaatcaatgcttcacccatacaacaaggtggccattctgttatttcagccatcagtcttcctttggttgatcaagatggaacaaccaaaattatcatcaactacagtcttgagcagcctagccaacttcaagttgttcctcaaaatttaaaaaaagaaaatccagtcgctacaaacagttgtaaaagtgaaaagttaccagaagatcttactgttaagtctgagaaggacaaaagctttgaagggggggtgaatgatagcacttgtcttctgtgtgatgattgtccaggagatattaatgcacttccagaattaaagcactatgacctaaagcagcctactcagcctcctccactccctgcagcagaagctgagaagcctgagtcctctgtttcatcagctactggagatggcaatttgtctcctagtcagccacctttaaagaacctcttgtctctcctaaaagcatattatgctttgaatgcacaaccaagtgcagaagagctctcaaaaattgctgattcagtaaacctaccactggatgtagtaaaaaagtggtttgaaaagatgcaagctggacagatttcagtgcagtcttctgaaccatcttctcctgaaccaggcaaagtaaatacccctgccaagaacaatgatcagcctcaatctgcaaatgcaaatgaaccccaggacagcacagtaaatctacaaagtcctttgaagatgactaactccccagttttaccagtgggatcaaccaccaatggttccagaagtagtacaccatccccatcacctctaaacctttcctcatccagaaatacacagggttacttgtacacagctgagggtgcacaagaagagccacaagtagaacctcttgatctttcactaccaaagcaacagggagaattattagaaaggtcaactatcactagtgtttaccagaacagtgtttattctgtccaggaagaacccttgaacttgtcttgcgcaaaaaaggagccacaaaaggacagttgtgttacagactcagaaccagttgtaaatgtaatcccaccaagtgccaaccccataaatatcgctatacctacagtcactgcccagttacccacaatcgtggccattgctgaccagaacagtgttccatgcttaagagcgctagctgccaataagcaaacgattctgattccccaggtggcatacacctactcaactacggtcagccctgcagtccaagaaccacccttgaaagtgatccagccaaatggaaatcaggatgaaagacaagatactagctcagaaggagtatcaaatgtagaggatcagaatgactctgattctacaccgcccaaaaagaaaatgcggaagacagaaaatggaatgtatgcttgtgatttgtgtgacaagatattccaaaagagtagttcattattgagacataaatatgaacacacaggtaaaagacctcatgagtgtggaatctgtaaaaaggcatttaaacacaaacatcatttgattgaacacatgcgattacattctggagaaaagccctatcaatgtgacaaatgtggaaagcgcttctcacactctgggtcttattctcaacacatgaatcatcgctactcctactgtaagagagaagcggaagaacgtgacagcacagagcaggaagaggcagggcctgaaatcctctcgaatgagcacgtgggtgccagggcgtctccctcacagggcgactcggacgagagagagagtttgacaagggaagaggatgaagacagtgaaaaagaggaagaggaggaggataaagagatggaagaattgcaggaagaaaaagaatgtgaaaaaccacaaggggatgaggaagaggaggaggaggaggaagaagtggaagaagaagaggtagaagaggcagagaatgagggagaagaagcaaaaactgaaggtctgatgaaggatgacagggctgaaagtcaagcaagcagcttaggacaaaaagtaggcgagagtagtgagcaagtgtctgaagaaaagacaaatgaagcctaatcgtttttctagaaggaaaataaattctaattgataatgaatttcgttcaatattatccttgcttttcatggaaacacagtaacctgtatgctgtgattcctgttcactactgtgtaaagtaaaaactaaaaaaatacaaaatacaaaacacacacacacacacacacacacacacacacacacacacacacaaaataaatccgggtgtgcctgaacctcagacctagtaatttttcatgcagttttcaaagttaggaacaagtttgtaacatgcagcagattagaaaaccttaatgactcagagagcaacaatacaagaggttaaaggaagctgattaattagatatgcatctggcattgttttatcttatcagtattatcactcttatgttggtttattcttaagctgtacaattgggagaaattttataattttttattggtaaacatatgctaaatccgcttcagtattttattatgttttttaaaatgtgagaacttctgcactacaaaattcccttcacagagaagtataatgtagttccggaa

SEQ ID NO: 34 - DNA, 3553 bp, human
ccacgcgtccggcgtcctccacataggcagtggctgtggtttctaccccgggtggccgggggcagtgctgagctgggactgttgtttgcccagcctgggctgcagaaagcagcagttaaagttcgtttctggtcactgctccaggaagccaccttactctgagggtcaagaattgccgcttccttttagttactgtaagttcctcctctgcccctggtttgtttcccgcggcacttctggatacccccaggtcccagacccttccagactcaaaccatgaactttcggcagctgctgttgcacttgccacgttatcttggagcctcgggttccccgcgtcgcctgtggtggtccccgtccctcgacaccatctcctcggtgggctcttggcgtggtcggtcctccaagtccccggcccactggaatcaggtagtgtcagaggcggagaagatcgtggggtaccccacgtccttcatgagccttcgctgcctgctgagcgacgagctcagcaacatcgctatgcaggtgcggaagctggtgggcactcagcaccctctgcttaccacagccagggggcttgtacatgacagctggaatagcctccagttgaggggcttggtggtgctccttatctctaaagcagctgggcccagcagcgtgaacacttcatgtcagaactatgacatggtcagtgggatctactcatgtcaaagaagtttggcagagatcacggagctaattcatattgctctccttgtacatcgtgggatagtaaatttaaatgagttgcaatcatctgatggtccactgaaagacatgcaatttggaaataaaattgctatcctgagtggagactttcttctagcaaatgcctgcaatggactagctctgctacagaacaccaaggttgtggaacttttagcaagtgctcttatggacttggtacaaggagtatatcatgaaaattctacttcaaaggaaagttatatcacagatgatattggaatatcgacttggaaggagcagacttttctctcccatggtgccttactagcaaagagctgccaagctgcaatggaattagcaaagcatgatgctgaggttcagaatatggcatttcagtatgggaagcacatggccatgagtcataagataaattctgatgtccagccttttattaaagaaaagaccagtgactccatgacttttaatctaaactcagctcctgtagtcttacatcaggaatttcttggaagagatttgtggattaaacagatcagagaggctcaagaaaaaggaagattggactatgctaagttgcgagaaagaatcaaagctggcaaaggtgtgacttcagctattgacctgtgtcgttaccatggaaacaaggcactggaggccctggagagctttcctccctcggaggccagatctgctttagaaaacattgtgtttgctgtgaccagattttcatgacatcaaattaaaaagacactattgttagttagctgaaaatcctagggaatgaggttgattgggagcgctttcacgatgcgttaatgacttttaaaacatatgcatttttccttccttttatcacattgctaaatgagttctgctttctttttggaactgctacaaacaaaattagaagaaaaaaaggtcaagcagttttcacttgtcacgccagaagcacacttgaggctgcagtcgcagaaataattaatgagattcgctcctgtgacctcagcaaatggacaggaaataagtccttattgattggaccgagccagggatggcgccagggcggtggcctgtggtttttcctgctagagaggacaaagcaagttggaagctgcaggtgtcaagagaaatgctctcaataccaaccagggaggattgtctaatcaaaaactagtgaccaatttgtcataatggagagtagttcaatggattgagaaaaatatgttttatttgttggcttgtaattatgtctctggattattattattttttttttagatgtagttttgctcttgttgcccaggctggagtgcaatggtgcagttttgactcactgcaacctccgcctcctgggttcaagtgattctcctgcctcagcctcccgagtagctggaattacaggcacctgccaccacgcctggctaattttgtatttttagtagagatggggtttcactatgttggtcaggctagtctcgaactcctgacctcaggtgatccgcctgcctcggcctcccaaagtgctgggattagaggcttgagccactgcacctggcctcatgtctctggatttataatgcagtatgaatatactttgtgctttatggtttttataatgtctttttggagaaattgccgaaaagttgccaaatacttgaagtaggagattaaaatgttatcaaatgttaaattggttatattaggaatagtctgtttttctttcctgaagatcagtttttttattcaaacacatttcaaagaaccaaatttttttttttctttaaggaaaaaggagctttttttcaagtgaaatgtattcatttgtaatactttggtttaaggcatactttaatttttacgagtttcagaaacagaatttttgtactagggaattcattggtgagagtgttcttttaacctcagaatgtcaaattttggtcttgaaccacagacatccaattacagaaagaatataagcaatctcacaggcctgcaatcggacactgtctctgtgtggttcataggagatgatttttgaggtttgcactcatgcaatttgagaacaccgttgacaagaaggctgagtttacataaatgatctagattgaaactcagctacctttcttcctcatgtggtgtaattacagccctatctggagacagcgaatacagcaaacagattttattacctagttcgctcaaacactacatgaagttattttagttaaagccctcccccaaaagttataaaaccattttatcagggcccaacatgtggcatgcaatgaagagaaaatgtaaagctacagaggttaatgtattgtattataaaatattttaagtgtactcaaaatatcataattgtacagtttatgccaccataatttgaggcctatagatttagcttaagagaacactgttctgtttgaaatgctttctgtcactgaaattggcttaattagtaaccatggataagatgctttagatcagactaggttttaatcattaacttccacaaagaagtcatactttgcgttaggtgtgctggttggatgtgcaggaacttcagcaagcagtaggttttactaagcagatggtcgggcactgcagggcaccaggcaggatcctagggcgcctcttattctgcgttagcatctggtttgctgtatgaccttgcacaagtcacttccttctgagcctcaattttctcatctgtacaatgagattcaaaagttgacctgaaagtcaagtgtgaaaaaaaaaaaagagattaaacaagataattatgaaattcaaaaaaaaaaaaaaa

SEQ ID NO: 35 - DNA, 7644 bp, human
agagccaagcggcgggctggcggcgggctccgacgtctgcgccaggacctggctggctgagcccggcgcagcagcagcagccagggcagcgcggcccctactccctgtcaggtcgtagaggcgagcagggaccagctggtcgccggcccctcgggcaagatggggaaccgggagatggaggagctgatcccgctggtgaaccgtctgcaggacgcgttttcggcgctgggacagagctgcctgctggagctgccgcagatcgccgtggtgggcggccagagcgccggcaagagctcggtgctcgagaacttcgtgggcagggactttctccctcgagggtcgggcattgtaacaagacgacctcttgtgctgcagcttgttacttctaaagcagaatatgccgagtttctacattgcaaaggaaagaaatttacagattttgatgaagttcgccttgagattgaagcagaaacagatcgcgtgactggaatgaataaaggcatttcctccatacccattaatttacgagtctattccccacacgtgttaaatctaacccttattgatctacctggaataactaaagtgcctgtgggagatcagccaccagatatcgagtatcagatcagagaaatgattatgcagttcatcacgagggagaactgtctgattttagctgttactccagccaacactgatcttgcaaactcagatgcgctgaagctagctaaagaagttgatcctcaaggtctgagaaccattggagttatcaccaaactggaccttatggatgaaggaacggatgccagggatgttctagagaacaaactgttgcctcttcgcaggggttacgtgggggtggtaaacagaagccagaaggacatagatgggaagaaggacataaaggcagccatgctggcagagaggaagtttttcctttcccacccggcttacagacatatcgctgaccgaatgggaaccccacacctgcagaaggtccttaatcagcaacttaccaaccacattcgggataccctaccaaacttcaggaacaaactacagggacagttgctctccatagaacatgaagtagaagcctacaaaaatttcaaaccagaagacccaacaaggaagaccaaagcattgctgcagatggttcagcaatttgctgtggactttgagaagagaattgaagggtcaggggatcaagtagataccctggaactctcaggtggtgctaaaatcaatcgtatttttcatgaacgctttccttttgagatagtaaagatggagttcaatgagaaagaattgcgaagagaaataagctatgcaatcaaaaacatacatggtatcaggacagggttgtttactccagacatggcatttgaagcgatagtcaagaaacagattgtaaagttgaaagggccttccttgaagagtgtggatctggtaatacaagaattaatcaacactgtgaagaagtgtaccaaaaaactggcaaacttccccagactctgcgaggaaacggaaaggattgttgctaaccacattcgtgagcgagaagggaagacaaaggaccaggtattgctattgattgacattcaagtctcttacatcaacaccaaccatgaagacttcattggcttcgcaaatgctcagcagaggagcagtcaggttcacaagaaaaccacagttggaaatcaggtgattcgcaaggggtggctcaccatcagcaacattggcatcatgaaaggcggctcgaagggatactggttcgtccttactgcggaaagcttgtcctggtataaagatgatgaggaaaaagaaaagaagtacatgcttcccttggacaacctgaaagttcgggatgtggaaaagagctttatgtctagcaagcacatctttgcactctttaatacagagcaaaggaatgtatacaaagactatcgcttccttgagctggcatgtgattcccaggaggatgtcgacagctggaaggcatctctactaagagctggggtctatcctgacaaatctgtagggaacaacaaagctgaaaatgatgagaatggacaagcagaaaacttttccatggacccacaattggagaggcaagtggagaccattcgcaacctcgtagactcctacatgtccattatcaacaaatgtatccgagatctaattccaaaaacaataatgcaccttatgatcaataacgttaaagatttcataaattccgagctcctagcacagttgtattcttcagaggaccaaaataccctgatggaggaatctgctgagcaggctcagcgccgggatgagatgcttcgaatgtatcaagcactgaaagaagcccttgggataattggggacatcagcacagccaccgtgtccactccggcaccccctccagtggatgactcctggatacagcactctcgcaggtcacctcctccaagccccacaacccaaaggaggccaacactaagtgctcccctcgcaaggcccacatccggccgaggaccagctcctgccattccctctcctggcccccactctggggctcctccagtcccattccgtccaggcccattacctcctttccccagcagcagtgactccttcggagcccctccacaagttccatctaggcctacgagggccccgcccagtgtcccaagccggagaccacccccatcaccaactcgtcccactataatccgcccactagaatcctccctgttagactaaacgaagtgtctggcatggcaattaatcactaatgaattatgcgaaagcaacatatttgataaccgttgcagtaaatcatgagtagtcgcatgtgtggacatcagtaggcaagtaaccagttttactaatgcattcatcgctcatcttcattgctcatggtatgtcaaacctttggggtttgactcagaaactgctaaccttttagaggctttatatgttgtactgaccaaggtaggtttgtatagcagccctatactttggggatcatttgcctaccatggcatatatttgaaattgctttggacaagttttctaggctatctaccaggtagctcattaaacgtaattcttcagatatgagatagtgggcttagacctaagccatacatatttcttttcccacattctgtttaggatgacagtaattctgttgtctaccattaatgctactacctactccataattgcctatttagctcctcttttcttcctttttatttcataagactgctaggaagtgattttttaaaattaggactccttaagaataaacttttccagaagcacgaggtagtttgcaaaggaaaagtctgcactgtttgctctaaagagcttctcctcattccaatgtgttttgcttcatgctagaagcatatgcaacagtgaataaaagcttctttttttgttaatcagtcaataaatttggctaattagtttcagagttcaaggaagaagcaaaatatcacatctctagaagtgttgggaaaaatataatttctttctttacttatattcacctcatggtaggttatattgaaggctgacatggagaatgtttacttttctatttggcatagctaactacactttgatactaactccagttttactattattattttggttggaaaaaaaattcactctttacgtgctaatttgtaatcttgttttgtaagaatttatcctacccttgaaacaggctcagtgtaactgtatatccattctaggctttcttaataaatcttgaggctatgggataatcacatttaaagaatggttcctgaaatgaagtcagtagaaatggcatgggataagagcagagctcacacttttacagttgcagtatttcaaagtccctatccaggtcactccagaaaagggtattgaaacgttgaaatctaaagcaaatttgcaatttcttaagatttctaaaatttaccagaacagtttagcctgggggttaatagttaagtcttgaggctaagttttggactaccaaggaccagatgattcacatgtaggaaacagccagaagccaactggaattttgtgtgctaactgttcccagacagcagagcaagtattcactgagtaggggtgtcccatgacactatttcatattctacagaagtaaatcaggtttcaccaactgaaatgtctccctttgaaagtagcaaacatgatttgtatgttaacttaactttaatttcctgtgtagtttacacccagagcagatactcataaagtataaagtaaaaacttttaaccattattaaacagagaacttgccatgttgagtgccattgtattgaacttattctaaaggcttatgctaacccatttataattggtaaaaatcagaaaatacaagatttacataaaggtcatttcaacttttaaggttaccagtgattgtataaaaacatcacaatcctaaatcctctcgtatctcaccccaaaccccaaactgggggaaaaaaagttaactctttgtgaatggaaccaatgtgcaagatacatactgcatttttaaaatagtgtctcagctaaatggaaaactgttaagcaaacatccatagtaaaacaaataatcttcagtgagatctttttataaaacttcttgtttttaggattccctttgcttcttcctttgaattctctaaaataggcagctaacggattatatacttcagggtttggctttgtgctaaatgtggttttgtgttttgctgtatttcaaaattttccttctgttaaaggaaaatattgtgaataaccactggtgtgttcttagatcagcacaaaccatgtcaaaaaaaattggagatttttttccaattttccttccactgatcttaggcagtaataaacaatggcatttgtcatctttggcacttgcttttagattatagtcccacagttgcactgccccaattgtctacctttgtgggtacatttttgttctttactcctaagttatttctcatagaacccagcctactctagaatttcagcagtgacattggagaatatttttaatttgctgcagtactatgtcatattattagtatgaatctcatttcccaaagggtttgtattctgctaaaaggagatgccaatgttgaatgaagtctgaaactctagtatgtgcatagtttgacgtgcagcatgcacaccaggccttaagatgggaatgtagcttaatgattttctgtttcccataccatttctaatcttttgtgtaattttctcttaactgattgctctgatattgtaaacacaatagatgtagctctatcatgtctagcataatttaaaaaatcagtgtttttaggatttgggaaaataaactgtaaatgtttatttgataggtaaatatagttttattgtcacatgctaaatattgcatgcatattgactaattggaataaccatttactcaattatggacagcttattgaaatagtattgatttagaaaaagtatattgcatttctaaaaaacatctaccaaggttactcgtctgaatattgcttttagccgtgttttataacatagacgagcagtagggtctgtttattagcaaatttcctatttgttccaatacaaactcactttattctaaagtatattaatgaaaccagttcctgtgatgtaactgtaagccttctcgacttagacttaaaaagtggtcacatagattaattttgtgactttttagtatagactgtagccataattctcaaatatgaaatgggacctaataccagtatgtgataaatgttgatgttttctgtgtacaaacacattttctatgcatgtgtctctgtgtatatggcatatacctagtaagtatgtttctgtaagtatgtgtattttatgtccatttgagtaggtaggtaggttttaaaaatactagttaaaatgccacaagcatgagtgtgattgtatgtgcactgtgtgtatatatataaatatatgtatatgtatggttgtaaatatctatgtatacatgtacttagtatgtgtggtatcaggatattttttaaactgtgataatacaacagatagctttgaatgatctgccataacatgtggtaacaatagttcatttctcataacatatatgaggtatacaaaattttctaactccagattgtagtcattttgagaggtacaaagctcataattaccatgacaacatggtaatgtccatagacatttgtatctgaatccacaagaagatctgaatctaagaaggaattacctttgacaatatttttcggtaagaagtaaaacctctggagacctatctttaagatctctaattggaattataaattatttttggattgctgagctgaatcttaaaaagccaagttgatatacatagtcatttttcctctatggtagaagtaaaaaaaaaaaaaaaggacatagcaacattaaagtagtggatttttctgagtaaatttgctgaaaatataagagagaagctatctaataccttggaggtaggtcatccactttttcaggtaaacatttttcatttggcaaatggcataattatttgaaagtgacaggaatctcctcagaatgaagaataaagcctaataggtctctaactgttgaactcatgaaagaagatagtgtatgagacttaagccatgagttttgtatcatttcaattagaagactactagctgtgagctcagagtttaatgtaaatgaatctagatgattttgaagaaatgattattcgttcaccagatcactcattgtacattctaaaaagctcaaatgagtcttctagatactcttactcatcctgtctggttgctatgtttaaaattatgtggtgctgtgtaggtgaaactttaagaatatttttgaagcatatgtaatatatgcactgctatttgtgtgtgtgtgtgtgtgtctttgtatatatgtaagaatgtgtgtatgtgtgagagcaagagagaggaaactcaaagaggagtgtttgtcttaagacctgttcatactggtatattggtgagacttctcacttctggttggaggtttcacatatggctcaactcaagtcattaatctctttttaatttttactcttgaattccttaaacttcgctcattatgaaatgttttaaaattatgacaaaaattactctgtctaaccacttgccttgtctgctaccagtttgttaaaaattattccccccaaccagtaattccaccagtactacttgatttgtgttatatttcctatgtacatgtacagcctttgttttgcttgcttgtctatttttactttcccttttttgggtcaaatttttcttttgctttgtttgaagaaggaatatacagaagtaaaatcttgtcttctctgctgattctttaattaatatgagccggatactttccactgtcttcttggcactttcaggatttcttaatgctgatatatggactcttagaatggaatttttgaagaaaaatctcaaagcctgtatcgttcttgaaggtcacatgtacctattgtgaaaatgtgaagctgtatttctgaagctgaaataaattataacatttgaaggaaaaaaaaaaaaaaaa

SEQ ID NO: 36 - DNA, 6054 bp, human
aacggcgggaaggctcaggggcgtgcttgcggcaaccctgtaaccgccggtgcgcaagttggaggggcaaggggctgccgtgtcctggtctggaggcggcccaggctgccttggtgattctggcttcgcggcctcatggcttgcggccggtttgcgcaacggcctgcgaaccaccaggcgcgtccccgccggcttgggtccgccccgaagcccagtccctcggcctggcccgctatgccctgcacactggcggagtctggcgtggcgcagcccggcgtggggttcggccccgccaggtggtcagcccttcctcggagtccaggtggctgggggcgcttcggagagtggccaggggcccccaaacccggggccgcagagcagtaccagtccgtggcctgttaggaaccaggctacagagcaggagttgccaaaacaaaggggatttggtgatggaggctttgttagaaggaatacaaaatcgagggcatggtgggggatttttgacatcttgtgaagcagaactacaggagctcatgaaacagattgacataatggtggctcataaaaaatctgaatgggaaggacgtacacatgctctagaaacttgcttgaaaatccgtgaacaggaacttaagagtcttaggagtcagttggatgtgacacataaggaggttggaatgttgcatcagcaggtagaagaacatgaaaaaatcaagcaagagatgaccatggaatataagcaggagttgaagaaactacatgaagaattatgcatactgaagagaagctatgaaaagcttcagaaaaagcaaatgagggaattcagaggaaataccaaaaatcacagggaagatcggtctgaaattgagaggttaactgcaaaaatagaggaattccgtcagaaatcgctggactgggagaagcaacgcttgatttatcagcaacaggtatcttcactggaggcacaaaggaaggctctggctgaacaatcagagataattcaggctcagcttgtcaatcggaaacagaaattagagtctgtggaactttctagccaatcagaaattcaacacttaagcagtaaactggagcgggctaatgacactatctgtgccaatgagttggaaatagagcgcctcaccatgagggtcaatgacttggttggaaccagtatgactgtcctacaggagcagcagcaaaaagaagaaaaattgagggaatctgaaaaactattagaggctctgcaggaagaaaagagagaattgaaggcagctcttcagtctcaagaaaatctcatacatgaggccagaatacaaaaggagaagttacaagaaaaagtaaaggcaactaacactcaacatgctgtagaagctataaggccacgggaagaatctctggcagaaaagaagtacacctctcaagggcagggggacttagacagtgtgctctcccagttgaattttacccatactagtgaggaccttctgcaggcagaggtgacttgtcttgaaggcagtttggaatctgtgagtgcaacgtgtaaacagctgagccaagaactaatggaaaaatatgaagaactgaagaggatggaagcacataacaatgaatacaaagcagagattaagaagttgaaagaacagattttacagggtgaacaaagttacagttctgcactagaaggaatgaagatggaaatctcccatctaactcaggagttacatcagcgagatatcactattgcttccaccaaaggttcttcctcagacatggaaaagcgactcagagcagagatgcaaaaggcagaagacaaagcagtagagcataaggagattttggatcagctggagtcactcaaattagaaaatcgtcatctttctgaaatggtgatgaaattggaattgggtttacatgaggcaaaagagatttcactagcagacctccaggagaattatattgaggcattaaataaattagtgtctgaaaatcaacaactacagaaagatttgatgaataccaaatctcagctggagatttctactcagatgtgcaaaaaacaaaatgacaggatctttaaaccaacacacagcagaacaactgagttcaagaatacagagttcaagccaacccatggccagcacagacatgatggaataaagactgagcactacaaaacagatcttcattctccaagaggacaagcgtcggatagtataaaccccatgtctagggtgctaagccccctgagtcctcaaatcagcccttgcagctccaccaggtctttgacttcctactctctatgtaaaactcattctttgccttcagcgctagatacaaatgaagccaatttttctgacactatgtctgagagtatgaatgaccaagaagagtttatatcttcgtgttccttgcctgtatctccccttggttcaatagctaccagatttttggaagaggaggaactgaggtctcatcacattctagagcgcttggatgcccatattgaagaactaaaaagagagagtgaaaagacagtgagacaattcacagccttaaagtagcctcttaaaaaaatcacaatcttggaaataaaaataaacaccaaagagttactgtcatctgaagtagcagctctttaaaaacatgaagagataaaattataaaaatgatacatctaaagcagtggtgaagaaagctgaaaaactgatacttttgataggcattttctctgcactggtttgtttaaaggacttcttccagcaataagttgaaagaataaaccactttgctagacttttttctcatacgaatatttattatcataaagtgatacttaccttgctgacttaaatgtgaatagctatgtactaattgaaataaggattttatgatacatgttgaaaataaagtaactgcaggaactttctttaggggaaatgtgtagaagcatggatttaggggtcaaacatacctggatcgatagactggttttgccacttaccagccaacggggcttgttatttactgggctggtagcccctcctagccaaggggctggtagtgtgtaaagtcaggctggtagtgaataaggggggggtgtgctaaagaaccttatcaagcagtcctctcttgctcaacactcatgcagaggagagaggcagggagggcaagggactggctgtcatgcacggtgcccatggaatatccattggaaataaatgttcatcgtctgcactgctgaggacaagtttagatgggagacaaagatctggatgttgatgtgccgcaggcatttgaaacgatgggagttgataagatccaccagggaggctgtggagagagagaggagcaggaggctgggtttggggccctgaaagatgccagcatctgaggatcacaggaaatagatctagcaaaggaactagaatgtgcagttacagatgagaaaggtcagtgtagtgtacttgccagctcaagggtgtggcctctgtgaaccctctttctgtagtggatctttcccatcagcgtaaaacatcctgttatttcccctttttaaataaaacctcagttttttttaatgcctctcttttactgctctgtatgtttgcttcccttcacccaaacacttctggaaagagttgtctgcagtgactctttccagttcctcaccccgccccaccgtagttctcttcctaaccctctccaacaccacttccatccccagactgctttcttgctcttgagttcaccagtgacctaatcagtgtctgagcatcattcagcacagttggactctctcttggcttctgtgacctggaacttctggtttcctctctacctcatcctccactccatctcatggcttttcctcttctaacctctaagtcagagggccccctagagctctgtcatgggttttctccctcagcattctcttccctagaagtttccattccattctcaagctcttaataccacctataggccagtgacccttacatttattactaatcttgacctctccccagagctgctgtcttagatacctagctgcttatgcgatgtcttcacttggatatctgacaaacatcttgtttccaagagagaactctttattctccctcttgctaagattccatatcaggacatagcattatgtttgccagttcctaggagttacttataattccttccttacccacatccgcatatccagtcatttgggaggtcctgtcatttcttcccccagaatcttatctcagacccaaccgtttccctcctgcacttctaccacctgatccagactacttctctctcaccctgattctggcagtagcctcctaactgatagtactcacaccagtaattcagggagcttctggagttatcctttagaaatgtaaatgaaacagtgtcaccactgcttcccttcagactcttccaaggcttctctttttttttttattgagacagagtctcactctgtcgcccaagctgcagtacagtggcaggatctcggctctctgcaacctccacctcccgggctcaagtgattctcctgccttagcttcccgagtagctgggattacaggcgcatgccaccaggcccagctaatttttgtatttttagtaggaacagggtttcaccatgttggtcaggctgatctcgaactcctgacctcaagtgatccacccgcctgagcttcccaaagtgctgggttataagcgtgagccactgcacccagccccaaggcttcttactgcactcagaataaaatccaaatgaattagtatagcctacagagtgtcaataatctgggcctaatcacccattgcatcatcctttactgtgctccacccatgctggcgtcctttctgtttattgagtgcagcaagctttaacctgttcgggtctttcatttgcagtttaccctgaatattccctttctcttacctcacctttgattggcgttttcccccttgcagacctgcactctggcatcctctcctgagcaagccttctttgattgctgcctcccaaacctgtttctccccatttctctagctcattggcctgtgttacttgcttcatagtgctcactactatctgaaatcatttcattacttgatttatgtttgtactgtctatttctgtctttagaattaaaactccttgagggcaaggactttgcttctctggttcatggtgggatcctcatcacccagcacaagcccaacacttaggagaggctcagctagtttgaaccaatggaaaatgccattcctgaggtgcaccacatcgtttcttttgtgttggtgtgcatgctgtccttcagtttgtctcctcttcccacaccctgtcatgtgctcctaaaactccccctttacttggctactttatctggcttggcaaagctttcccatatcccctgacccatgtcagggaacgtttctcctcattgctgtcatggcaccctgtgtgtggctctgaccagactttgctatccgctttgatttctgtctttcaggggctaagtcctgcctacatcctcagtcagcatgctgcctaacacataacagatcctaagcaaatattgggtggatgaaaatcactaccattcaaggcttcattgtatttatcagtaaaactgaaataatatctaatggggtcgagaagatttactgaaaatatatatttgaaagggctttgcagttttaagtactgtacctatgtgcattgctgttacattcaggagatttctgagttttagacagtcccaagcttcagcttaaaatctgtagactgcagcccgtttctgaaacgtttgtactacgtgttgactaggaggaacaatgacagactctgtagtcaccggaaatacttaaggaatcatgaggtactatgtatttccttaaattatactgttttttaaagctgaagtatctaatagttaatgggttgtccaaatttgtcagaacatttgactgtaatttaatgtctcaccatttttcaaaaagatatattaataccaaatattaaaatgtgtcaagact

SEQ ID NO: 37 - DNA, 2276 bp, human
cctgtgtgctggggaggaatcccgcagtggccggggggcttgaggccgctgctttgtctcttcgtccagagccttatgtaagagcttttctcgggaaacaggaagtcctgcttgccaatttcagcacagggagtagtgcaggccttattccaacacacccggcccagccttaaccccagaactcagccagtttcttgcttccgtgcccctggttctcctccccatcgagcccacccctcctttcccaccttcagtcacccctagtgaactgccccagcgatctctgctgtgcttgaccccgagggtcttccaccctcgccctgaccctggacactgcccagcttggccccccatcctgctcctggcacaatgccctctagccagccaaccttccctcccccaaccctggggccgccccagggttcctgcgcactgcctgttcctcctgggtgtcactggcagccctgtccttcctagagggactggaacctaattctcctgaggctgagggagggtggagggtctcaaggcaacgctggccccacgacggagtgccaggagcactaacagtacccttagcttgctttcctcctccctcctttttattttcaagttcctttttatttctccttgcgtaacaaccttcttcccttctgcaccactgcccgtacccttacccgccccgccacctccttgctaccccactcttgaaaccacagctgttggcagggtccccagctcatgccagcctcatctcctttcttgctagcccccaaagggcctccaggcaacatggggggcccagtcagagagccggcactctcagttgccctctggttgagttggggggcagctctgggggccgtggcttgtgccatggctctgctgacccaacaaacagagctgcagagcctcaggagagaggtgagccggctgcaggggacaggaggcccctcccagaatggggaagggtatccctggcagagtctcccggagcagagttccgatgccctggaagcctgggagaatggggagagatcccggaaaaggagagcagtgctcacccaaaaacagaagaagcagcactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggtttgtgaaactgtgattgtgttataaaaagtggctcccagcttggaagaccagggtgggtacatactggagacagccaagagctgagtatataaaggagagggaatgtgcaggaacagaggcgtcttcctgggtttggctccccgttcctcacttttcccttttcattcccaccccctagactttgattttacggatatcttgcttctgttccccatggagctccgaattcttgcgtgtgtgtagatgaggggcgggggacgggcgccaggcattgtccagacctggtcggggcccactggaagcatccagaacagcaccaccatctagcggccgctcgagggaagcacccgccggttggccgaagtccacgaagccgccctctgctagggaaaacccctggttctccatgccacacctctctccaggtgccctctgcctcttcaccccacaagaagccttatcctacgtccttctctccatctatcggaccccagtttccatcactatctccagagatgtagctattatgcgcccgtctacagggggtgcccgacgatgacggtgccttcgcagtcaaattactcttcgggtcccaaggtttggctttcacgcgctccattgccccggcgtggcaggccattccaagcccttccgggctggaactggtgtcggaggagcctcgggtgtatcgtacgccctggtgttggtgttgcctcactcctctgagctcttctttctgatcaagccctgcttaaagttaaataaaatagaatgaatgataaaaaaaaaaaaaaa

SEQ ID NO: 38 - DNA, 3877 bp, human
ggcggtcgcgcgcaggactcgagggcttctagccaccgtccccgccagcgccgcgccccgccacagggcggcatgagcccacccgcggccgcagccctagcgccctgctcctccgcctgggcggcccggctgcggtgacggctctcgctgcccgactgggggccatgaagccgagtccggccgggacggcgaaggagctggagcctccggcgccggcccgaggcgagcagcgcacggcggagcccgaggggcgctggcgggagaagggcgaggcagacaccgagcggcagcgcacccgggagcggcaggaggccacgctggccgggctggcggagctggagtacctgcgccagcgccaagagctgctggtcaggggcgccctgcgcggcgccgggggtgcgggagccgctgcgccccgcgctggggagctactgggggaggcggcgcagcgcagtcgcctggaggagaagttcttggaggagaacatcttgctgctaagaaagcaattgaactgtttgaggcgaagagatgctggtttgttgaatcagttgcaagagcttgacaagcagataagtgacctgagactggatgtagaaaagacatctgaagagcacctggagacagacagtcggcctagctcagggttttatgagctgagtgatggggcttcaggatccctttccaattcctctaactcggtgttcagtgagtgtttatccagttgtcattccagcacctgcttttgcagccccttggaggcgaccttgagtctctcagatggttgccccaaatctgcagatctcataggattgttggaatataaagaaggccactgtgaagaccaggcctcaggggcagtttgccgttccctctccacaccacaatttaattcccttgatgtcattgcagatgtgaatcccaagtaccagtgtgatctggtgtctaaaaacgggaatgatgtatatcgctatcccagtccacttcatgctgtggctgtgcagagcccaatgtttctcctttgtctgacgggcaaccctctgagggaagaggacaggcttggaaaccatgccagtgacatttgcggtggatctgagctagatgccgtcaaaacagacagttccttaccgtccccaagcagtctgtggtctgcttcccatccttcatccagcaagaaaatggatggctacattctgagcctggtccagaaaaaaacacaccctgtaaggaccaacaaaccaagaaccagcgtgaacgctgaccccacgaaagggcttctgaggaacgggagcgtttgtgtcagagccccgggcggtgtctcacagggcaacagtgtgaaccttaagaattcgaaacaggcgtgtctgccctctggcgggataccttctctgaacaatgggacattctccccaccgaagcagtggtcgaaagaatcaaaggccgaacaagccgaaagcaagagggtgcccctgccagagggctgcccctcaggcgctgcctccgaccttcagagtaagcacctgccaaaaacggccaagccagcctcgcaagaacatgctcggtgttccgccattgggacaggggagtcccctaaggaaagcgctcagctctcaggggcctctccaaaagagagtcctagcagaggccctgccccgccgcaggagaacaaagttgtacagcccctgaaaaagatgtcacagaaaaacagcctgcagggcgtccccccggccactcctcccctgctgtctacagctttccccgtggaagagaggcctgccttggatttcaagagcgagggctcttcccaaagcctggaggaagcgcacctggtcaaggcccagtttatcccggggcagcagcccagtgtcaggctccaccggggccacaggaacatgggcgtcgtgaagaactccagcctgaagcaccgcggcccagccctccaggggctggagaacggcttgcccaccgtcagggagaaaacgcgggccgggagcaagaagtgtcgcttcccagatgacttggatacaaataagaaactcaagaaagcctcctccaaggggaggaagagtgggggcgggcccgaggctggtgttcccggcaggcccgcgggcgggggccacagggcggggagcagggcgcatggccacggacgggaggcggtggtggccaaacctaagcacaagcgaactgactaccggcggtggaagtcctcggccgagatttcctacgaagaggccctgaggagggcccggcgcggtcgccgggagaatgtggggctgtaccccgcgcctgtgcctctgccctacgccagcccctacgcctacgtggctagcgactccgagtactcggccgagtgcgagtccctgttccactccaccgtggtggacaccagtgaggacgagcagagcaattacaccaccaactgcttcggggacagcgagtcgagtgtgagcgagggcgagttcgtgggggagagcacaaccaccagcgactctgaagaaagcgggggcttaatttggtcccagtttgtccagactctgcccattcaaacggtaacggccccagaccttcacaaccaccccgcaaaaacctttgtcaaaattaaggcctcacataacctcaagaagaagatcctccgctttcggtctggctctttgaaactgatgacgacggtttgagtgacatcattggtgtagaaagtttgtgtgtttttttttcttctccctagttgccaaaattaaaaaggtggtgttttcatttttgtataatactttaatggaatgctttttaaaaaaatataaaaccaaggtaaattattgtttcatcttcacgtatggatgctagtgcctttaatggaaggtaaagaatgttttgctagttagaagtacatattgaggttttaatggtggtgatagtgagttttgtggcaccagctgttttttattttaaactttctgagcatccggcaaggtacaggttttgatgttcaagttttattgggataagatcttttgatcccaaggtcaggtggatggaatttttggatttatatttgttccttgagtcttcagggcagtgtctccatgagggttttcctgttgaggggcaccacatacaatagtgtgaagtaggtatgaggggcagtcattgtattctatagtttttttatgtagtctacatttctcagatgtatccccattcggttttattctcagaactgttactagactcatgacttggaggccaaaccttaaatccagagatagcagcctcgatagggaccttaaaaggattcacaaaaacttttgccacacttggtgcctaggccctgttcctaataaccccttctagggccgtttatccaacatttagatgccttcttttccctccctaatttgtagccagtccaacctttcattccttggaggatttagttttgggataaaattttggtccttgggcacagagacattcactattaatgaagtaacccttgggcatgactccaatcccagaattgctcactgagcgctatgccaccgaagcgttgacctgaacatattagtgcaatccagtccagattggacctttgatcctatgtggaagggctgttttttaagaaaaaatttttggtaaacagtattgtgtaaaattgctttttgtataccaatatatgcatgttttgtgcatgagtagtacttgtgttgatactcctgttgatgttaaattactatataatataaacagtatgtgtttttatatatcattgtgtaaatttaatataacatatgcagtaataaaccatttgttttactgctgttaagtttgttatttgggtataaaaccagatgtttacacctgtaaaaaaaaaaaaaaaaaa

SEQ ID NO: 39 - DNA, 4414 bp, human
cccacgcgtccggcggcggcggtagcggcggcagcggctgcggccaagctgggtccgagcatcccgcggctctggagccgcccggcccggacatggcgaccgtccgggcctctctgcgaggtgcgctgctccttctgctggccgtggcgggggtcgcggaggtggcagggggcctggctccgggcagtgcgggtgcattgtgttgtaatcattcaaaggataaccaaatgtgccgtgatgtatgtgaacagattttctcctcaaaaagtgaatcccgactaaaacatctgttgcagcgagccccagattattgcccagagacaatggttgaaatttggaattgtatgaattcatctttgccaggtgtgtttaagaagtctgatggctgggttggcttaggctgctgtgaactggctattgccttggagtgtcgacaggcatgcaagcaggcatcttcaaagaatgatatttccaaagtttgcagaaaagaatatgagaatgctcttttcagttgcattagcagaaatgaaatgggctcggtttgttgcagttatgcaggtcatcacacaaactgccgagaatactgtcaagccatttttcgaacagactcttctcctggtccatctcagataaaagcagtggaaaattattgcgcctctattagtccacaattaatacattgtgtgaacaattatactcaatcttatccaatgaggaacccaacggatagtttatattgctgtgacagagctgaagaccatgcttgccaaaatgcctgcaagagaatcctgatgtccaagaaaacggaaatggagattgttgatggtctcatcgagggttgtaagacccagcccttgcctcaagatcctctttggcaatgttttcttgaaagctcacaatctgttcaccctggagtcactgtacaccctcctccctctacaggcctcgatggggctaaattgcattgttgttctaaagcaaacacttcaacatgtagggaactctgcactaaactttacagcatgagctggggcaatacacagagttggcaagagtttgatcgcttttgtgaatataatccagtggaagtgtccatgttgacctgtttagcggatgtccgggaaccttgccagttgggctgtagaaaccttacttactgtactaattttaacaacaggccaacagaacttttcaggagttgtaatgcacagtcagatcaaggagccatgaatgacatgaagttgtgggagaaaggaagcataaagatgccatttatcaatatacctgttcttgatattaaaaagtgccagccagagatgtggaaagcaatagcttgttcactgcagattaaaccttgtcatagtaaatctcggggaagtattatttgcaaatcagattgtgtggagattcttaaaaaatgtggagaccagaacaaattccctgaagaccacacagctgaaagtatttgtgagcttctgtcacctacagatgatctgaagaattgtatacctttggatacatacctcaggccaagtactttaggtaacattgtagaagaagtgactcatccctgtaacccaaatccttgccctgccaatgagctctgtgaagtaaaccgaaaaggatgtccatctggagatccctgtcttccatacttttgtgttcaaggttgcaaactgggagaagcttctgatttcattgtccgtcaagggacactaatccaggtgccatcatctgcaggggaagttggttgttataaaatctgttcatgtggacaaagtggactcttagaaaactgtatggaaatgcactgtatagacctccagaagtcttgtattgttggaggaaaaagaaaaagtcatggaacatcctttagtattgactgcaatgtctgttcttgttttgctggcaatttggtgtgctctacccgcctttgcctcagtgagcacagttcagaagatgaccgtcgtaccttcacaggtctgccctgtaactgtgcagatcagtttgtccctgtatgtgggcagaatgggcgcacttaccccagtgcctgcattgctcgctgtgtgggcctccaagaccatcagtttgagtttggatcatgcatgtcaaaggatccatgtaatcctaatccctgccaaaaaaaccaaaggtgcatacccaaaccacaggtctgcctgacgacttttgataaatttggatgtagccagtatgagtgtgtaccaagacagctcgcgtgtgaccaggtccaagatcctgtttgtgacacagaccacatggagcacaacaatctctgcactttataccaaagaggaaaaagcctctcttacaaaggtccctgccagcccttttgcagagcaaccgagcccgtatgtgggcacaatggtgagacctacagcagtgtgtgtgctgcctactcggatcgcgtggcagtcgattactatggggactgccaggccgtcggagtcctctcagagcacagctccgtcgccgagtgtgcttctgtcaagtgtccttcgctcttggcagctggatgcaaacccatcatcccaccgggtgcttgttgcccattatgtgctgggatgttaagagttttatttgacaaagaaaaactggatactattgctaaggtaacaaataaaaagccaataacagttctggaaatacttcagaaaatccgcatgcacgtgtctgtcccacagtgtgatgtgtttggatacttcagcattgaatcagaaattgtgatcctgatcattcccgtcgatcactatccaaaagctctgcagattgaagcctgcaataaagaagcagagaagattgagtcccttatcaactctgacagcccgactttggcgtcccatgtccctctctctgccctcatcatttcccaggtacaggtctccagcagtgtgccatcggccggtgtcagggccaggccttcttgccactccctcctccttcccctcagcttgggccttgccttgcacttgctctggacatataactgactgcccacggaaagtgcagaatgctcctccacctcactctcctgccttgaaaaagacattcaggactgctggtttgtagttgaatattggccaaggaaaggcacatgtcacctctattcgccacacagtatttttttttttaatccgccaatattagtaggatttttgttttgtttttacaaatgttaaaatgtgttgttccaaatactaatgaaaacagaatgtctcttcctggtagaccactgccatatgatttacatttcctcaccataagggtcccccactctaaagcaaatttatcgctgggaaatgagatgaccactttttagaaagataattcactgtactatcaggttcacaaacttcatttcagagttctttttgaagtatttaaggttcccgttgcatttgttttgtttacagataattacctactctggctagaagctaggggtcccagtgaagagccactgccattaaagaatatgaaacatagataaaacatctttgaaattatgtaaattatgtaaattatcaggcaaatttgcattaaattacagaaatttaattcagaaccccaactactgtgttatgcaaaagcaagctgattaaatgacactcatataattatatgttgtaagcaacaggctcactggtcacggatttgtgtctgtgacttttgtgaaagggagaagtgacattgcatcaaagcatcttgcattatgcaatttttatattaaccagatatatattcatcggtattcatccaagttaaatgtagagtttttaaacatcaatctttaaaccaattgctgctacttatataattgccaaaaagtgaaataatgtgtagttcatgtaaataatacattatatttctattttattatgaagaaggtgaatagccatatttgtaaaatgacaatcatgtgtgttaacccagtgctttccattcgtgaaaacacatttgctttttgtgatatgcacaatgtagataagtgttctgtctgactttcttttttgatatagaagtataaagaattgtggtttatatatttaaaagtgtcaagctgagtattaaaatgtatgcatgttgtctaagaaattgaatacttgaatgtgtctcacagtttgaaataagctatttgatgtaatacttcttgtgtgtatgcacatgaaacttagattttacatgaagtattttttcagtattatatgtaccctctgaaatacatagggatatgcgtattataccaaaatgttgctgaaaaatgggcacttaaagctttcagaatatgtcagtgctgatgtagcatgcttgttgcaattgccttttttctgtataaatgtctttaatgcaatatactggaaagcttttctattttaataaaaataatttttatatg

SEQ ID NO: 40 - DNA, 419 bp, human
ctggggaaattgcttcactgtcttagaaccaagcaagattcacctgtgttttgtgttcatgttcatttgcctcttaaaggcaagggttgaagataaataaggtagcaatgtctatagttttggccttaactatgccaatctaattataattccctgtatttaaaatggtttcttttacttattgaaaggcattttagtgtggtttatgtgtaatattaaagattattcaacacctctcacatcttacagatctataaggtcacatgcttttaaaatagtagcaagttaaacttcactcttgaattctttacaatctaagtcaaactaagttataatttaggattgtctttaaacagccattcagaaacaaaactgtagaactgtgtatttgattgggaatggtgcttttgccaacttaa

SEQ ID NO: 41 - DNA, 496 bp, human
ggctctggaggtgggcagcaatctgtcaatcaccttgttaaagagattgatatgctgctgaaagaatatttactctctggagacatatctgaagctgaacattgccttaaggaactggaagtacctcattttcaccatgagcttgtatatgaagctattataatggttttagagtcaactggagaaagtacatttaagatgattttggatttattaaagtccctttggaagtcttctaccattactgtagaccaaatgaaaagaggttatgagagaatttacaatgaaattccggacattaatctggatgtcccacattcatactctgtgctggagcggtttgtagaagaatgttttcaggctggaataatttggaaacaactcagagatctttgtccttcaaggggcagaaagcgttttgtaagcgaaggagatggaggtcgtcttaaaccagagagctactgaatataagaactcttgcagtcttagatgttat

SEQ ID NO: 42 - DNA, 338 bp, human
tgtttggctgtagcagtgcggccctggctgtgcatggaaacctggagggggctggcatcgtgctcaagtacatcatggctggttgccccttgtttctgggtaatctctgggatgtgactgaccgcgacattgaccgctacacggaagctctgctgcaaggctggcttggagcaggcccaggggccccccttctctactatgtaaaccaggcccgccaagctccccgactcaagtatcttattggggctgcacctatagcctatggcttgcctgtctctctgcggtaaccccatggagctgtcttattgatgctagaagcctcataactgttctacctc

SEQ ID NO: 43 - DNA, 524 bp, human
cctgtgcagctccgattttattcgcatccttgtgatcttctctggaatgttccttgttttcaccctggccggggccctgttcctccatcaacgaaggaaatatagatcaaacaaaggagaaagtcctgtggagcctgcagagccttgtcgttacagctgccccagggaggaggagggcagcaccatccccatccaggaggattaccgaaaaccggagcctgcctgctccccctgagccagcacctgcggtagctgcactacagccctggcctccacccccaccccgccgaccatccaagggagagtgagacctggcagccacaactgcagtcccatcctcttgtcagggccctttcctgtgtacacgtgacagagtgccttttcgagactggcagggacgaggacaaatatggatgaggtggagagtgggaagcaggagcccagccagctgcgcctgcgctgcaggagggcgggggctctggttgtaaaacacacttcctgctgcgaaagacccacatgctaca

SEQ ID NO: 44 - DNA, 347 bp, human
  misc_feature (34)..(34): n is a, c, g, or t
tgtttggctgtagcagtgcggccctggctgtgcntggaaacctggagggggctggcatcgtgctcaagtacatcatggctggttgccccttgtttctgggtaatctctgggatgtgactgaccgcgacattgaccgctacacggaagctnnnnnnnnnnnnnnnnttggagcaggnccaggggccccccttctctactnnnnnnnnnnnnnnnnnnnnnnnnnnnnnnnnnnnnatcttattggggctgcacctatagcctatggcttgcctgtctctctgcggtaaccccatggagctgtcttattgatgctagaagcctcataactgttctacctccaaggttag

SEQ ID NO: 45 - DNA, 186 bp, human
gaaattccggacattaatctggatgtcccacattcatactctgtgctggagcggtttgtagaagaatgttttcaggctggaataatttggaaacaactcagagatctttgtccttcaaggggcagaaagcgttttgtaagcgaaggagatggaggtcgtcttaaaccagagagctactgaatataa

SEQ ID NO: 46 - DNA, 264 bp, human
atgcatgtttcggggacttggcccttctgatgaggggccctcggtactctggataacgaagcttgtgcagagtggtaaccatgcttacacactaaactataatataaaggaaatgaagccatgttaatctgagagcagtgtcgccatagttgtgttgtttacaatactctataaatgggttcctgttgccctgtaattaaactgctgcccgtagaggcctttcagttccttttctgtccttccccttcttaacacaagctcaaa

SEQ ID NO: 47 - DNA, 441 bp, human
tttcgagggggcaaggagggacagaaaagtaacctcttcttaagtggaatattctaataagctaccttttgtaagtgccatgtttattatctaatcattccaagttttgcattgatgtctgactgccactcctttctttcaaggacagtgttttttgtagtaaaatcactggtttatacaaagctttatttagggggtaaagttaagctgctaaaaccccatgttggctgctgctgttgagatactgtgctttgggagtaaaaaaagaaagttatttctttgtcttaaagaatttttaaaaaattagtcatgagacttattcatctttccagggaacatactgattggtcttaaaagactagacagttaagtaaaaggtggctggaacatctatttttctacaaaactggaaaaatgaacctggttctagaagaatgtaca

SEQ ID NO: 48 - DNA, 525 bp, human
ctggctgggcgtgtaggacggtgagagcacttctgtcttaaaggttttttctgattgaagctttaatggagcgttatttatttatcgaggcctctttggtgagcctggggaatcagcaaaaggggaggaggggtgtggggttgataccccaactccctctacccttgagcaagggcaggggtccctgagctgttcttctgccccatactgaaggaactgaggcctgggtgatttatttattgggaaagtgagggagggagacagactgactgacagccatgggtggtcagatggtggggtgggccctctccagggggccagttcagggcccagctgccccccaggatggatatgagatgggagaggtgagtgggggaccttcactgatgtgggcaggaggggtggtgaaggcctcccccagcccagaccctgtggtccctcctgcagtgtctgaagcgcctgcctccccactgctctgccccaccctccaatctgcactttgatttgcttcctaacagctctgtt

SEQ ID NO: 49 - DNA, 354 bp, human
  misc_feature (221)..(221): n is a, c, g, or t
aacttgtcacgaattccatcttgtaaaggatattaaatgttgctttaacctgaaccttgagcaaattagttggtttgtgtgatcatacagttatgtgggtggcttctagtttgcaacttcaagggacaagtattaatagttcagtgtatggcgttggtttgtgttgagcgtttgcacggtttggataatcttaaattttgacggacactgtggagactttnctgttactaaatccttttgttttgaagctgttgctatttgtatttctcttgtcctttatattttttgtctgtttatttacgcttttattggaaatgtgaataagtaaagaattacttgtgttacttgccaagc

SEQ ID NO: 50 - DNA, 510 bp, human
  misc_feature (48)..(48): n is a, c, g, or t
ggtgggatccctaacgtaatattctttatgaaagcattagctgctttngttacatttttaatatgcaaccacttcttcacctgaggaaaactagaatgaaatgcagtctaaaatattttgcactgaattgtaatttcttcattagtttagtctggaaactggtctgttttaatgtgttttttaaatgctgtatgtagaggaaaatctgcagaccactggaatacatttgttaaactctcatctgcagggacactgggcgatacttggcagtgactgttctaccttgaggctttgtttggtttatttattaaagtgtacagtatttaaaaatcaaacatagctttagttaaaacactaagctgaattagtcatgtccattcagacataacctgaactactgaaaagatcaatttccagaaggtttattctgtataaactacatgttagtcttcagtagagtatcttttttttttccttttttttttttttatttcggttgtttgatgtgca

SEQ ID NO: 51 - DNA, 406 bp, human
gataaaacggcaacacagctcacaagaacagactttccagctgctgaagttatggaaacatcaaaacaaagcccaagatatagtcaagaagatcatccaagatattgacctctgtgaaaacagcgtgcagcggcacattggacatgctaacctcaccttcgagcagcttcgtagcttgatggaaagcttaccgggaaagaaagtgggagcagaagacattgaaaaaacaataaaggcatgcaaacccagtgaccagatcctgaagctgctcagtttgtggcgaataaaaaatggcgaccaagacaccttgaagggcctaatgcacgcactaaagcactcaaagacgtaccactttcccaaaactgtcactcagagtctaaagaagaccatcaggttccttcacagc

SEQ ID NO: 52 - DNA, 465 bp, human
gatgagtgagtctccccttaattagactgcaaatgtcacttgtgatgagtgtgccattccaggataacagcttgcaccctcctcagaatgttttcagcgaaagagtggggtggctgttctctgctcctggtgctttggcctcatttcacactattagaattctggggctgtaaggccagccagtgtcagctcatgttccattggctctccacctgccatttttagggagctattccttatatagttacaaattcccttgtcatttacttatttggaaacatgggatttactctgacaagctttagcctatgttatgggattcagaacaatgagatcataataattctcactgaccaaagctgggactccatcctgccatttttgtgtggagatattcataattctgcaatactttaaaacatttagaaaacaccccagggtaggtctgtggcccttagacagtga

SEQ ID NO: 53 - DNA, 164 bp, human
  misc_feature (105)..(105): n is a, c, g, or t
ggttccatcaatggtgagcaccagcctgaatgcagaagcgctccagtatctccaagggtaccttcaggcagccagtgtgacactgctttaaactgcatttttctnaatgggctaaacccagatggtttcctaggaaatcacaggcttctgagcacagctgcatt

SEQ ID NO: 54 - DNA, 462 bp, human
ggctgtttgcccaggaacttagggtgggtacctctgagtcccagggacctgggcaggcccaagcccaccacgagcatcatccagtcctcagccctaatctgcccttaggagtccaggctgcaccctggagatcccaaacctagccccctagtgggacaaggacctgaccctcctgcccgcatacacaacccatttcccctggtgagccacttggcagcatatgtaggtaccagctcaaccccacgcaagttcctgagctgaacatggagcaaggggagggtgacttctctccacatagggagggcttagagctcacagccttgggaagtgagactagaagaggggagcagaaagggaccttgagtagacaaaggccacacacatcattgtcattactgttttaattgtctggcttctctctggactgggagctcagtgaggattctgaccagtgacttacac

SEQ ID NO: 55 - DNA, 561 bp, human
  misc_feature (27)..(27): n is a, c, g, or t
cctctcagcaggctgtgtgtgccaggnctgtggggcagaggacgtggtgcccttccagtgccctgcctgtgacttccagcgctgccaagcctgctggcaacggcaccttcaggcctctaggatgtgcccagcctgccacaccgcctccaggaagcagagcgtcatgcaggtcttctggccagagccccagtgagtgcccacggaggcccccagcacacccaacgtggcttgatcacctgcctgtccagctctggtgggccaagaacccacccaacagaataggccagcccatgccagccggcttggcccgctgcaggcctcaggcaggcggggcccatggttggtccctgcggtgggaccggatctgggcctgcctctgagaagccctgagctaccttggggtctggggtgggtttctgggaaagtgcttccccagaacttccctggctcctggcctgtgagtggtgccacaggggcaccccagctgagcccctcaccgggaaggaggagacccccgtgggcacgtgtccacttttaatcaggggacagggctctctaata

SEQ ID NO: 56 - DNA, 312 bp, human
agatctgtgcggttggcataaccaacttactaacagaatgtcccccaatgatggacactgagtataccaaactgtggactccattattacagtctttgattggtctttttgagttacccgaagatgataccattcctgatgaggaacattttattgacatagaagatacaccaggatatcagactgccttctcacagttggcatttgctgggaaaaaagagcatgatcctgtaggtcaaatggtgaataaccccaaaattcacctggcacagtcacttcacatgttgtctaccgcctgtccaggaagggttc

SEQ ID NO: 57 - DNA, 397 bp, human
aatatccttgttgtgtattaggtttttaaataccagctaaaggattacctcactgagtcatcagtaccctcctattcagctccccaagatgatgtgtttttgcttaccctaagagaggttttcttcttatttttagataattcaagtgcttagataaattatgttttctttaagtgtttatggtaaactcttttaaagaaaatttaatatgttatagctgaatctttttggtaactttaaatctttatcatagactctgtacatatgttcaaattagctgcttgcctgatgtgtgtatcatcggtgggatgacagaacaaacatatttatgatcatgaataatgtgctttgtaaaaagatttcaagttattaggaagcatactctgttttttaatca

SEQ ID NO: 58 - DNA, 498 bp, human
ttggcatggaggcgccattgtgtcggctttgagccagactgggtctctctttaagccccggaaagcctatctgccccaggagttgctgggaaaggaaggatttgatgccctggatccctttattcccatcctggtttccaactatacccaaaggaatttgaaagttgtattcagtattatttggaaaacaattggcttcaacatgagaaagctcctacagaagaagggaaaaaagagctgctgttcctaagtaacgcgaacccctcgctgctggagcggcactgtgcctacctctaagccaagatcacagcatgtgaggaagacagtggacatctgctttatgctggacccagtaagatgaggaagtcgggatgacacaggaagaggagccaggcccttgtacctatgggattggacaggactgcagttggctctggacctgcattaaaatgggtttcactgtgaatgcgtgacaataagatattcccttgttcctaa

SEQ ID NO: 59 - DNA, 472 bp, human
gagaggagcaacggggtgggggcacactgtgtgcccagctgccagccctctggaaagccttccagccgctggtgaagaactaccagccctgtaatggcatggccaggaagcccagcagagcttataagatctaacccttatggccaggtgcagtggctcacgtatgtaatcccagcactttgggaagccaaggagggaggatcacttgaactcaggaattcgagaccagcctgggctacatagtgagaccacatctctactagaacttaaaaaaagttagccaggcacggtgataaatgcctgtagtcccagccactgaagccagaggatcgattgaaccagggagatcatggtcacagtgaactatgattacgccaacctgggtcacatagcaagactctgtttcaaaaaaaagggggggcgggggacgggtgggtgcagtggctcacatctgtaaccccagcactttggg

SEQ ID NO: 60 - DNA, 509 bp, human
gctcaattgctctgagctcagccaagtaggagaggctaggccatcactcttgggaagctgtgtagtgatgatgtataagaatcctcctcactgtcatgggatgttgtatccagcccctccttgttccagccggtggtgtgacttcgttggttgaggtgtgtctccaacctacatcagaccatgaagttcaacccctccagggaagctcctgatttcccctgcataattgaaaataggatattctcagctattgaacagttactaatttatggggtggaaacagcattaagaatactgaatcaaatggaaaaacaaatgaatacaggaagataagtgttcgttcttttctgaaaaaagagtatgtgtaccacaagagctggttttaattgggtgaattgtttttgtcctcattctgtacagaaatttgtatatatgatggttcttagaacttgttttaatttttgtggtccttctgtttattataataggcgtccaccaatgattatcca

SEQ ID NO: 61 - DNA, 553 bp, human
gggtgtcccgtgaggctgtatcgggtctgctgatcatgggagcgggcggaggctccctcatcgtcctctccatgctgctcctgcgcaggaagaagccctacggggctatcagccatggcgtggtggaggtggaccccatgctgaccctggaggagcagcagctccgcgaactgcagcggcacggctatgagaaccccacttaccgcttcctggaggaacgaccctgacccggccctcttcacccctagccgagcccagacctcccctcttcctggagccccagaaccccaactcccagcctagggcagcagggagtcttgaaagtgatcatttcacacccttttgtgagacggctggaaattcttatttcccctttccaattccaaaattccatccctaagaattcccagatagtcccagcagcctccccacgtggcacctcctcaccttaattttttaagtttatttatggctctttaaggtgaccgccaccttggtcctagtgtctattccctggaattcaccctctcatgtttccctactaacatcccaa

SEQ ID NO: 62 - DNA, 446 bp, human
gccaagcggaccagagctgtgcagcccctcaggctacctagcaacaaggcccaggtgaagccagggcagacatgcagtgtggccggctgggggcagacggcccccctgggaaaacattcacacacactacaagaggtgaagatgacagtgcaggaagatcgaaagtgcgaatctgacttacgccattattacgacagtaccattgagttgtgcgtgggggacccagagattaaaaagacttcctttaagggggactctggaggccctcttgtgtgtaacaaggtggcccagggcattgtctcctatggacgaaacaatggcatgcctccacgagcctgcaccaaagtctcaagctttgtacactggataaagaaaaccatgaaacgctactaactacaggaagcaaactaagcccccgctgtaatgaaacaccttctctggagcca

SEQ ID NO: 63 - DNA, 439 bp, human
cccacgtgggattcacaggggcctgagtagggcccggggaagcagagccctaagggattaaggctcagacacctctgagagcaggtgggcactggctgggtacggtgccctccacaggactctccctactgcctgagcaaacctgaggcctcccggcagacccacccaccccctggggctgctcagcctcaggcacggacagggcacatgataccaactgctgcccactacggcacgccgcaccggagcacggcaccgagggagccgccacacggtcacctgcaaggacgtcacgggcccctctaaaggattcgtggtgctcatccccaagcttcagagaccctttggggttccacacttcacgtggactgaggtagaccctgcatgaagatgaaattatagggaggacgctccttccctcccctcctagaggagagga

SEQ ID NO: 64 - DNA, 474 bp, human
actgggctactctggcgcagaaattaggtctggggatacttaataatgccttccggctgagtcctgctccttccaaaacacttatggacaactatgaggtctctgggggtacagtcagagagctggtggaggccctgagacaaatgggctacaccgaagcaattgaagtgatccaggcagcctccagcccagtgaagaccacctctcaggcccactcgctgcctctctcgcctgcctccacaaggcagcaaatagacgagctccgagacagtgacagtgtctgcgacacgggcgtggagacatccttccgcaaactcagctttaccgagtctctgaccagtggtgcctcactgctaactctcaacaaaatgccccatgattatgggcaggaaggacctctagaaggcaaaatttagcctgctgacaatttcccacaccgtgtaaaccaaagccctaaaattcactgcgttgtcc

SEQ ID NO: 65 - DNA, 529 bp, human
gatgagcagtcacactgttactccacagcggaggagaccagctcagaggcccaggaatcggagcgaagcagagaggtggagaactgggatttgaacccccgccatccttcaccagagcccatgctcaaccactgtggcgttctgctgcccctgcagttggcagaaaggatgttttgtcccatttccttggaggccaccgggacagacctggacactagggtcaggcggggtgctgtggtggggagaggcatggctggggtgggggtggggagacctggttggccgtggtccagctcttggcccctgtgtgagttgagtctcctctctgagactgctaagtaggggcagtgatggttgccaggacgaattgagataatatctgtgaggtgctgatgagtgattgacacacagcactctctaaatcttccttgtgaggattatgggtcctgcaattctacagtttcttactgttttgtatcaaaatcactatctttctgataacagaattgccaaggcagcgggatctcgt

SEQ ID NO: 66 - DNA, 198 bp, human
catacgctggacgagtcggaccgaggctaggacgtggccggcgctctccagccctgcagcagaagaacttcccgtgcgcgcggatcctcgctccgttgcacgggcgccttaagttattggactatctaatatctatgtatttatttcgctggttctttgtagtcacatattttatagtcttaatatcttgtttttgca

SEQ ID NO: 67 - DNA, 542 bp, human
ctccctagcataaatttcccaaacagtccacaggtcccatgcccactttgcgtctgcactgtgatcgtgacaaatcttccctcctcaccagctagtctggggtttcctctccctgccccaggccagaactgccttcttcatttccacccacgctcccagcctcttagctgaaagcacaaatggtgaaatcagtagtctcgctccatctctaatagactaaacctaaatgcctctaggacggactgttgctatccaagcgtttggtgttaccttctcctgggaggtcctgctgcaactcaagttccacaggatggtcaagctgtcagacatccaagtttacatcattgtaattattactggtatttacaatttgcaagagttttgggttagtttttttttttttttttttgctttgtttttgtacaaaagagtctaacattttttgccaaacagatatatatttaatgaaaagaagagatacataaatgtgtgaatttccagttttttttttaattattttaatcccaaacatcttcctgaaa

SEQ ID NO: 68 - DNA, 550 bp, human
gaaagtgtcctatttgtaggagtacaatcaagggtacagttcgtacatttctttcatgaagaagaaccaaaacatcatctaaactttagaattaatttattaaatgtattataactttaacttttatcctaatttggtttccttaaaatttttatttatttacaactcaaaaaacattgttttgtgtaacatatttatatatgtatctaaaccatatgaacatatattttttagaaactaagagaatgataggcttttgttcttatgaacgaaaaagaggtagcactacaaacacaatattcaatcaaaatttcagcattattgaaattgtaagtgaagtaaaacttaagatatttgagttaacctttaagaattttaaatattttggcattgtactaatacctggttttttttttgttttgtttttttgtacagacagggcagcatactgagaccctgcctttaaaaacaaacagaacaaaaacaaaacaccagggacacatttctctgtcttttttgatcagtgtcctatacatcgaaggtgtgcata

SEQ ID NO: 69 - DNA, 482 bp, human
gccatccagcacgtcatcaattatgacatgccagaggagattgagaactatgtacaccggattggccgcaccgggcgctcgggaaacacaggcatcgccactaccttcatcaacaaagcgtgtgatgagtcagtgctgatggacctcaaagcgctgctgctagaagccaagcagaaggtgccgcccgtgctgcaggtgctgcattgcggggatgagtccatgctggacattggaggagagcgcggctgtgccttctgcgggggcctgggtcatcggatcactgactgccccaaactcgaggctatgcagaccaagcaggtcagcaacatcggtcgcaaggactacctggcccacagctccatggacttctgagccgacagtcttcccttctctccaagaggcctcagtccccaagactgccaccagtctacacatacagcagccccctggacagaatcagcatttcagctcagctggcctgg

SEQ ID NO: 70 - DNA, 540 bp, human
aactcagctcttccctattgggaagagttatgcaaattctcctataagcaaaacaaagcatgtctttgagtaacaatgacctggaaatacccaaaattccaagttctcgatttcacatgccttcaagactgaacaccgactaaggttttcatactattagccaatgctgtagacagaagcattttgataggaatagagcaaataagataatggccctgaggaatggcatgtcattattaaagatcatatggggaaaatgaaaccctccccaaaatacaagaagttctgggaggagacattgtcttcagactacaatgtccagtttctcccctagactcaggcttcctttggagattaaggcccctcagagatcaacagaccaacatttttctcttcctcaagcaacactcctagggcctggcttctgtctgatcaaggcaccacacaacccagaaaggagctgatggggcagaatgaactttaagtatgagaaaagttcagcccaagtaaaataaaaactcaatcacattcaattccaga

SEQ ID NO: 71 - DNA, 522 bp, human
  misc_feature (261)..(264): n is a, c, g, or t
tacccctgggtctataactaccagctggaagggatctttgcccaggaattccccgacttggagaatgtggtcaagggccaccacgttagccaagaaccctggaacagcagcatcacactcacatcccaggccggggctgttttccagagctttgccaagttcagcaaatttggagatgacctgtactccggctggttggcagcagcccttggtaccaacctgcaggtccagttctggcacaaaactgtaggcatcctgccnnnnannngnnngnannnntggcaggttctgaatgtgaaccagatagctttccctggaccagccggcccaagcttcaacagcacagaggaccactccaaatggtgcgtgtccccaaaagggccctggacctgcgtgggtgacatgaatcngaaccagggagaggagcaacggggtgggggcacactgtgtgcnnanntgccagccctctggaaagccttccagccgctggtgaagaactaccagccctgtaatggcatggcc

SEQ ID NO: 72 - DNA, 459 bp, human
agcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagcgaggtggacattcatctgtttccactgagtctgagtcttcaagttttcactccagctaacaca

SEQ ID NO: 73 - DNA, 347 bp, human
cagggcagtgcctgctcccagaatctggtgctctgaccaggccaggtggggaggctttggctggctgggcgtgtaggacggtgagagcacttctgtcttaaaggttttttctgattgaagctttaatggagcgttatttatttatcgaggcctctttggtgagcctggggaatcagcaaaaggggaggaggggtgtggggttgataccccaactccctctacccttgagcaagggcaggggtccctgagctgttcttctgccccatactgaaggaactgaggcctgggtgatttatttattgggaaagtgagggagggagacagactgactgacagccatgggtggt

SEQ ID NO: 74 - DNA, 548 bp, human
cagctgcttattactgtggcattcaatcaacctgttaagctttattccatgaaatttcaagggccagataatggtcagggccctaaatatgtaaaaatttttatcaacctaccccgatctatggattttgaagaggcagaaagaagtgaaccaactcaagctctggaactgacagaggatgatattaaagaagatggcattgttccacttcgttatgttaagtttcagaatgttaacagtgtaactatatttgttcagtcgaatcaaggtgaagaggaaacaacaagaatttcatattttacttttattggtactccagtccaggcaacaaatatgaatgacttcaaacgagtagttggcaaaaaaggagaaagccactaaggtacaaaagacactggacaaccatattgcaatcagatctacagctcctggataattgcttgattctcgccagagactgtcaatgtttcattcattgccattaccaataaatcattgcttttgttcagatggtatcactagtgtttccattgtaatcttgacacatg

SEQ ID NO: 75 - DNA, 543 bp, human
gctacctcaggcgtgagcacgctcagttctactgtgtctcatgattcccagagtgctcaccggtcactgaatgtacagcttggaaggatgaaaatagaaaccaatagattactggaagaattggttcggaaagagaaagaattacaagcactccttcatcgagctattgaagaaaaagaccaagaaattaaacacctgaagcttaagtcccaacccatagaaattcctgaattgcctgtatttcatctaaattcttctggcacaaatattgaagattctgaacttaccgactggctgagagtgaatggagctgatgaagacactataagccggtttttggctgaagattatacactattggatgttctctactatgttacacgtgatgacttaaaatgcttgagactaaggggagggatgctgtgcacactgtggaaggctatcattgactttcgaaacaaacagacttgactgttgctcaatctaatcttcgatggaaattctaaaaattaatacagagctgatcttcttgggggtgggaaa

SEQ ID NO: 76 - DNA, 479 bp, human
  misc_feature (441)..(441): n is a, c, g, or t
accaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctagagtgaaaaacaacaaattcagttctgagtatatgcaattagtgtttganaagattcttaatagctggctgtaaatactgcttggttt

SEQ ID NO: 77 - DNA, 463 bp, human
  misc_feature (61)..(61): n is a, c, g, or t
tttgcacttccttcggagagcatctaagattggagaggttgatgtcgagcaacatactttngccaaatacctgatggaactaactatgttggactatgacatggtgcactttcctccttctcaaattgcagcaggagctttttgcttagcactgaaaattctggataatggtgaatggacaccaactctacaacattacctgtcatatactgaagaatctcttcttccagttatgcagcacctggctaagaatgtagtcatggtaaatcaaggacttacaaagcacatgactgtcaagaacaagtatgccacatcgaagcatgctaagatcagcactctaccacagctgaattctgcactagttcaagatttagccaaggctgtggcaaaggtgtaacttgtaaacttgagttggagtactatatttacaaataaaattggcaccatgtgccatctgtacata

SEQ ID NO: 78 - DNA, 485 bp, human
ggtttcatttgctcttgggggattaggtatcatttggggaggaagcatgtgttctgtgaggttgttcggctatgtccaagtgtcgtttactaatgtacccctgctgtttgcttttggtaatgtgatgttgatgttctccccctacccacaaccatgcccttgagggtagcagggcagcagcataccaaagagatgtgctgcaggactccggaggcagcctgggtgggtgagccatggggcagttgacctgggtcttgaaagagtcgggagtgacaagctcagagagcatgaactgatgctggcatgaaggattccaggaagatcatggagacctggctggtagctgtaacagagatggtggagtccaaggaaacagcctgtctctggtgaatgggactttctttggtggacacttggcaccagctctgagagcccttcccctgtgtcctgccaccatgtgggtcagatgtactctctgtcacatg

SEQ ID NO: 79 - DNA, 222 bp, human
catacccatattttaagggacaggtgaatttactactttctaaagtttattgatacttcccttttatgtaaaatgtagtagtgatacctatatttccacattgtgcattgtgacacacttgtctagggatgcctggaagtgtataaaattggactgcatttcttagagtgttttactatagatcagtctcatgggccatctcttcctcagatgtaaatgata

SEQ ID NO: 80 - DNA, 421 bp, human
  misc_feature (257)..(257): n is a, c, g, or t
cacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccnatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgt

SEQ ID NO: 81 - DNA, 461 bp, human
gaagctgttgcaatggagtcttatgctaaagcactgagaatgttgccaaccatcatagctgacaatgcaggctatgacagtgcagacctggtggcacagctcagggctgctcacagtgaaggcaataccactgctggattggatatgagggaaggcaccattggagatatggctatcctgggtataacagaaagttttcaagtgaagcgacaggttcttctgagtgcagctgaagcagcagaggtgattctgcgtgtggacaacatcatcaaagcggcacccaggaaacgtgtccctgatcaccacccctgttaagcattcccacgtgctgtcgatctttggaccagtttctagcaaagttgtgtttgaaagatactctattaaagaagactgtggaatctgtttatcggtgcccattatatccttaagtttggatatttagctgaccttcgctttaacat

SEQ ID NO: 82 - DNA, 482 bp, human
agaaaaccacacttctcataccttcactcaacacttccttccccaaagccagaagatgcacaaggaggaacatgaggtggctgtgctgggggcaccccccagcaccatccttccaaggtccaccgtgattaacatccacagcgagacctccgtgcccgaccatgtcgtctggtccctgttcaacaccctcttcttgaactggtgctgtctgggcttcatagcattcgcctactccgtaaagtctagggacaggaagatggttggcgacgtgaccggggcccaggcctatgcctccaccgccaagtgcctgaacatctgggccctgattctgggcatcctcatgaccattggattcatcctgttactggtattcggctctgtaacagtctaccatattatgttacagataatacaggaaaaacggggttactagtagccgcccatagcctgcaacctttgcactccactgtgcaatgctggcc

SEQ ID NO: 83 - DNA, 489 bp, human
agaggatatccattcctgagctcctggctcatccatatgttcaaattcaaactcatccagttaaccaaatggccaagggaaccactgaagaaatgaaatatgttctgggccaacttgttggtctgaattctcctaactccattttgaaagctgctaaaactttatatgaacactatagtggtggtgaaagtcataattcttcatcctccaagacttttgaaaaaaaaaggggaaaaaaatgatttgcagttattcgtaatgtcagataggaggtataaaatatattggactgttatactcttgaatccctgtggaaatctacatttgaagacaacatcactctgaagtgttatcagcaaaaaaaattcagtgagattatctttaaaagaaaactgtaaaaatagcaaccacttatggcactgtatatattgtagacttgttttctctgttttatgctcttgtgtaatctacttgacatcattttactct

SEQ ID NO: 84 - DNA, 377 bp, human
ttgaaagttgtgggtcagctgaccaggtagaggattcaagactcaatgtggaaaaaatattttaaactactgattgaatgttaatggtcaatgctagcacaatattcctatgctgcaatacattaaaataactaagcaagtatatttatttctagcaaacagatgtttgttttcaaaatacttctttttcattattggttttaaaaaagcattatccttttatctcacaaataagtaatatctttcagttattaaatgatagataatgcctttttggttttgtgtggtattcaactaatacatggtttaaagtcacagccgtttgaatatattttatcttggtagtacattttctcccttaggaatatacatagtct

SEQ ID NO: 85 - DNA, 567 bp, human
gcggcctattgctttagaaatgtgcaggataattgctgcctacgtccactttacattgatttcaagagggatctagggtggaaatggatacacgaacccaaagggtacaatgccaacttctgtgctggagcatgcccgtatttatggagttcagacactcagcacagcagggtcctgagcttatataataccataaatccagaagcatctgcttctccttgctgcgtgtcccaagatttagaacctctaaccattctctactacattggcaaaacacccaagattgaacagctttctaatatgattgtaaagtcttgcaaatgcagctaaaattcttggaaaagtggcaagaccaaaatgacaatgatgatgataatgatgatgacgacgacaacgatgatgcttgtaacaagaaaacataagagagccttggttcatcagtgttaaaaaatttttgaaaaggcggtactagttcagacactttggaagtttgtgttctgtttgttaaaactggcatctgacacaaaaaaagttgaaggccttattctacatttcacctactttgta

SEQ ID NO: 86 - DNA, 384 bp, human
ctcttgaaaggcactgtcatttaaacataaacctggagtactcgaaatagaattcaggtttacaagatgaaagcgtgtggagaagtgtcagatggcagtggaagcatgtgtgtttctaaaaagtaaaaatctcaagaaaacagaaatggcatgctttacccatcttacttagtgaaagagagctgcagttgaaattgtttaaaaagtagcaggtacaatgaatattgtcacagatgtgttaatttttgaagcaatgtgggtgctgactactagtagtatcaaaaatatgttcaggattgttttgatacctgtatttataataaaaaatgttggggggagttgatgaattcctgttaaaagctgttcttgtgtgttacatgtaac

SEQ ID NO: 87 - DNA, 261 bp, human
aaatgctcttgaaccggaaacccagggatgggagatgctcactgagctgctgcttttatgtgtgctggtgctatgtgtgttcatgtccgcggcagctgtctttttgctactataagggaattctggccaccctgggtggggtgtggtcggggtgagaacccaagcgttggaactgtagacccgtcctgtcgactgtgtgcccctgggcatgtgtgagcctcagtttcctcatctgtaaggggggcaatgatacctacctca

SEQ ID NO: 88 - DNA, 434 bp, human
  misc_feature (48)..(48): n is a, c, g, or t
taagggaatatccctgcgctccagacgccaaaataagactgaggcagnacagcaaataactgaggtctttgtattagcagaaagaatagaaataaacagaaatgaaaagaagcccatgaagacctccccagagatggacattcagaatccagatgatggagcccggaaacccatacctagagacaaagtcantgagaacaaaaggtgcttgaggtctgctagacagaatgagagctcccagcctaaggtggcagaggagagcggagggcagaagagtgcgaaggttctcatgcagaatcagaaagggaaaggagaagcaggaaattcagactccatgtgcctgagatcaagaaagacaaaaagccagcctgcagcaagcactttggagagcnaatctgtgcagagagtaacgcggagtgtcaagaggtgtgcag

SEQ ID NO: 89 - DNA, 503 bp, human
aaactggcatctgctctagattcataaatacaaaaatgaatactgaattttgagtctatcctagtcttcacaactttgacgtaattaaatccaacttttcacagtgaagtgcctttttcctagaagtggtttgtagactcctttataatatttcagtggaatagatgtctcaaaaatccttatgcatgaaatgaatgtctgagatacgtctgtgacttatctaccattgaaggaaagctatatctatttgagagcagatgccattttgtacatgtatgaaattggttttccagaggcctgttttggggctttcccaggagaaagatgaaactgaaagcatatgaataatttcacttaataatttttacctaatctccacttttttcataggttactacctatacaatgtatgtaatttgtttcccctagcttactgataaacctaatattcaatgaacttccatttgtattcaaatttgtgtcataccagaaagctctacatt

SEQ ID NO: 90 - DNA, 464 bp, human
gtgggttcttattgattcgctgtgtcatcacatcagctccactgttgccaaactttgtcgcatgcataatgtatgatggaggcttggatgggaatatgctgattttgttctgcacttaaaggcttctcctcctggagggctgcctagggccacttgcttgatttatcatgagagaagaggagagagagagagactgagcgctaggagtgtgtgtatgtgtgtgtgtgtgtgtgtgtgtgtgtgtgtatgtgtgtagcgggagatgtgggcggagcgagagcaaaaggactgcggcctgatgcatgctggaaaaaagacacgcttttcatttctgatcagttgtacttcatcctatatcagcacagctgccatacttcgacttatcaggattctggctggtggcctgcgcgagggtgcagtcttacttaaaagactttcagttaattctcactggtatcatcgca

SEQ ID NO: 91 - DNA, 504 bp, human
gccactacacttcttaaggcgagcatcaaaagccggggaggttgatgttgaacagcacactttagccaagtatttgatggagctgactctcatcgactatgatatggtgcattatcatccttctaaggtagcagcagctgcttcctgcttgtctcagaaggttctaggacaaggaaaatggaacttaaagcagcagtattacacaggatacacagagaatgaagtattggaagtcatgcagcacatggccaagaatgtggtgaaagtaaatgaaaacttaactaaattcatcgccatcaagaataagtatgcaagcagcaaactcctgaagatcagcatgatccctcagctgaactcaaaagccgtcaaagaccttgcctccccactgataggaaggtcctaggctgccgtgggccctggggatgtgtgcttcattgtgccctttttcttattggtttagaactcttgattttgtacatagtcctctggtctatctcatgaaac

SEQ ID NO: 92 - DNA, 462 bp, human
ctgtcaaggccgtagcatggtgtccctggcagtccaatgtcctggcaacaggagggggcaccagtgatcgacacattcgcatctggaatgtgtgctctggggcctgtctgagtgccgtggatgcccattcccaggtgtgctccatcctctggtctccccattacaaggagctcatctcaggccatggctttgcacagaaccagctagttatttggaagtacccaaccatggccaaggtggctgaactcaaaggtcacacatcccgggtcctgagtctgaccatgagcccagatggggccacagtggcatccgcagcagcagatgagaccctgaggctatggcgctgttttgagttggaccctgcgcggcggcgggagcgggagaaggccagtgcagccaaaagcagcctcatccaccaaggcatccgctgaagaccaacccatcacctcagttgttttttat

SEQ ID NO: 93 - DNA, 395 bp, human
gaacctggtgaaagtcccatattctggtatgctccagaatcactgacagagagcaagttttctgtggcctcagatgtttggagctttggagtggttctgtatgaacttttcacatacattgagaagagtaaaagtccaccagcggaatttatgcgtatgattggcaatgacaaacaaggacagatgatcgtgttccatttgatagaacttttgaagaataatggaagattaccaagaccagatggatgcccagatgagatctatatgatcatgacagaatgctggaacaataatgtaaatcaacgcccctcctttagggatctagctcttcgagtggatcaagtaagggataacatggctggatgaaagaaatgaccttcattctgagaccaaag

SEQ ID NO: 94 - DNA, 396 bp, human
  misc_feature (310)..(310): n is a, c, g, or t
tcaacaccctcttcttgaactggtgctgtctgggcttcatagcattcgcctactccgtgaagtctagggacaggaagatggttggcgacgtgaccggggcccaggcctatgcctccaccgccaagtgcctgaacatctgggccctgattctgggcatcctcatgaccattggattcatcctgttactggtattcggctctgtgacagtctaccatattatgttacagataatacaggaaaaacggggttactagtagccgcccatagcctgcaacctttgcactccactgtgcaatgctggccctgcacngctngggctgttgcccctgcccccttggtcctgcccctagatacagcagtttatacccacacacctgtctacagtgtcattcaata

SEQ ID NO: 95 - DNA, 463 bp, human
gatcccggtgcagctgaatgccggccagctgcagtatatccgcttagcccagcctgtatcaggcactcaagttgtgcagggacagatccagacacttgccaccaatgctcaacagattacacagacagaggtccagcaaggacagcagcagttcagccagttcacagatggacagcagctctaccagatccagcaagtcaccatgcctgcgggccaggacctcgcccagcccatgttcatccagtcagccaaccagccctccgacgggcaggccccccaggtgaccggcgactgagggcctgagctggcaaggccaaggacacccaacacaatttttgccatacagccccaggcaatggcacagccttcctccccagaggacccggccgacctcagcgcctcctgcaggctaggacactggtgcactacaccccatgcctggggccgagattctccagcagaa

SEQ ID NO: 96 - DNA, 390 bp, human
gaaggtgtggttttcatttctcagtcaccaacagatgaataattatgcttaataataaagtatttattaagactttcttcagagtatgaaagtacaaaaagtctagttacagtggatttagaatatatttatgttgatgtcaaacagctgagcaccgtagcatgcagatgtcaaggcagttaggaagtaaatggtgtcttgtagatatgtgcaaggtagcatgatgagcaacttgagtttgttgccactgagaagcaggcgggttgggtgggaggaggaagaaagggaagaattaggtttgaattgctttttaaaaaaaaaagaaaagaaaaagacagcatctcactatgttgccaaggctcatcttgagaagcaggcgggttgggtggg

SEQ ID NO: 97 - DNA, 361 bp, human
aattactaagtcatctgtattaaattttgttataatgtaaagagattcaagccttgtaaataatgacatggaagaccctgtgctgcacttaaaatattgctgcttgattatttgatttttacatcagagctttataacacgaacttttgtacagaattgtgagttgtgaccatgtaacatgagaggttttgctagggcctattatttttaaccaccattaattagttggggtggagtttactgtaatgtgaaattttcacatttgaattttttaattgcctggcaaaagctgatataagttctaaaatatcagcagaatgatttgctgaattcattacaaccctgttatgtcactttttga

SEQ ID NO: 98 - DNA, 533 bp, human
ccctcaatctagaacgctacacaagaaatattttgtttttactcagcaggtgtgccttaacctccctattcagaaagctccacatcaataaacatgacactctgaagtgaaagtagccacgagaattgtgctacttatactggaacataatctggaggcaaggttcgactgcagtcgaaccttgcctccagattatgaaccagtataagtagcacaattctcgtggctactttcacttcagagtgtcatgtttattgatgtggagctttctgaatagggaggttaaggcacacctgctgagtaaaacaaatatttcttgtgtagcgttcttaggaatctggtgtctgtccggccccggtaggcctgttgggtttctagtcctccttaccatcatctccatatgagagtgtgaaaataggaacacgtgctctacctccatttagggatttgcttgggatacagaagaggccatgtgtctcagagctgttaagggcttatttttttaaaacattggagtcatagcatgtgtgtaa

SEQ ID NO: 99 - DNA, 268 bp, human
tgccctgaccccgatcagttaaggagctgtgcaataaccttcctagtacctgagtgagtgtgtaacttattgggttggcgaagcctggtaaagctgttggaatgagtatgtgattctttttaagtatgaaaataaagatatatgtacagacttgtattttttctctggtggcattcctttaggaatgctgtgtgtctgtccggcaccccggtaggcctgattgggtttctagtcctccttaaccacttatctcccatatgagagtgtg

SEQ ID NO: 100 - DNA, 546 bp, human
gctgtagtgttgaatacttggccccatgagccatgcctttctgtatagtacacatgatatttcggaattggttttactgttcttcagcaactattgtacaaaatgttcacatttaatttttctttcttcttttaagaacatattataaaaagaatactttcttggttgggcttttaatcctgtgtgtgattactagtaggaacatgagatgtgacattctaaatcttgggagaaaaaataatattaggaaaaaaatatttatgcaggaagagtagcactcactgaatagttttaaatgactgagtggtatgcttacaattgtcatgtctagatttaaattttaagtctgagattttaaatgtttttgagcttagaaaacccagttagatgcaatttggtcattaataccatgacatcttgcttataaatattccattgctctgtagttcaaatctgttagctttgtgaaaattcatcactgtgatgtttgtattctttttttttttctgtttaacagaatatgagctgtctgtcatttacctactt

SEQ ID NO: 101 - DNA, 539 bp, human
aagggcagtttgctggatttcctgaagagcgatgaaggtggcaaagtgctgcttccaaagctcattgacttttctgctcagattgcagagggaatggcatacatcgagcggaagaactacattcaccgggacctgcgagcagctaatgttctggtctccgagtcactaatgtgcaaaattgcagattttggccttgctagagtaattgaagataatgagtacacagcaagggaaggtgctaagttccctattaagtggacggctccagaagcaatcaactttggatgtttcactattaagtctgatgtgtggtcctttggaatcctcctatacgaaattgtcacctatgggaaaattccctacccagggagaactaatgccgacgtgatgaccgccctgtcccagggctacaggatgccccgtgtggagaactgcccagatgagctctatgacattatgaaaatgtgctggaaagaaaaggcagaagagagaccaacgtttgactacttacagagcgtcctggatgatttctacacagc

SEQ ID NO: 102 - DNA, 503 bp, human
gagttccttcaagaagttcctctgataggaagctagaagtgtagaatgaagttttacttgacagaaggacctttacatggcagctaacagtgctttttgctgaccaggattggtttatatgattaaattaatatttgcttaataatacactaaaagtatatgaacaatgtcatcaatgaaacttaaaagcgagaaaaaagaatatacacataatttctgacggaaaacctgtaccctgatgctgtataatgtatgttgaatgtggtcccagattatttctgtaagaagacactccatgttgtcagctttgtactctttgttgatacatgcttatttagagaagggttcatataaacactcactctgtgtcttcaacagcatctttctttccccatctttctattttctgcaccctctgcttgttccctcatattctgttcttccgactcctgctaacacacatgcaacaaaaaagggaagggagtgcttatttccctttgtgt

SEQ ID NO: 103 - DNA, 468 bp, human
atagcgtattatcacttgtcaaatcattgaaattcatttggggtcaaagacttgagtgacccagtattgagccatgaataatttagtgtaacctgtattacaagtacattgatgaattctgtatcttctttggtttcctgtatctttttaatcaagtctagaaactatgttcatcagtcactcatttttaaggtcgggagttagattttatgatagaattatgactgttagcttttctccttatagcatcttagtcttagaaattggtgggttgtaataatcaagggcttcattccttttatgtcatttctagacagttttgaatctaggttaataacactttatttataaagcacctcaatgtcctgtgaacactaattattttaaatgtgttaatactgtgcctttgatttgttagctttaaagttagtttaagacttttacactgccagtattccacatttggtg

SEQ ID NO: 104 - DNA, 397 bp, human
atgtggtgggtatcaggaggcagcggcttaagggcgatgcctgggtttacaaaagattagtggaagacatcctatctagctgcaaggtataattgatggattcttccatcctgccggatgagtgtgggtgtgatacagcctacataaagactgttatgatcgctttgattttaaagttcattggaactaccaacttgtttctaaagagctatcttaagaccaatatctctttgtttttaaacaaaagatattattttgtgtatgaatctaaatcaagcccatctgtcattatgttactgtcttttttaatcatgtggttttgtatattaataattgttgactttcttagattcacttccatatgtgaatgtaagctcttaactatgtctctttgtaa

SEQ ID NO: 105 - DNA, 468 bp, human
  misc_feature (399)..(399): n is a, c, g, or t
tgctaagttcaagtttcgtaatgctttgaagtatttttatgctctgaatgtttaaatgttctcatcagtttcttgccatgttgttaactatacaacctggctaaagatgaatatttttctactggtattttaatttttgacctaaatgtttaagcattcggaatgagaaaactatacagatttgagaaatgatgctaaatttataggagttttcagtaacttaaaaagctaacatgagagcatgccaaaatttgctaagtcttacaaagatcaagggctgtccgcaacagggaagaacagttttgaaaatttatgaactatcttatttttaggtaggttttgaaagctttttgtctaagtgaattcttatgccttggtcagagtaataactgaaggagntgcttatcttggctttcgagtctgagtttaaaactacacattttgacatagtgtttattagcagccatc

SEQ ID NO: 106 - DNA, 505 bp, human
attgctgaactcttaggcacatcaattaattgattcctcgcgacatctttctcaaccttatcaaggattttcatgttgatgactcgaaactgacagtattaagggtaggatgttgctctgaatcactgtgagtctgatgtgtgaagaagggtatcctttcattaggcaagtacaaattgcctataatacttgcaactaaggacaaattagcatgcaagcttggtcaaacttttcccaggcaaaatgggaaggcaaagacaaaagaaacttaccaattgatgttttacgtgcaaacaacctgaatcttttttttatataaatatatatttttcaaatagatttttgattcagctcattatgaaaaacatcccaaactttaaaatgcgaaattattggttggtgtgaagaaagccagacaacttctgtttcttctcttggtgaaataataaaatgcaaatgaatcattgttaacacagctgtggctcgtttgagggattggggtgga

SEQ ID NO: 107 - DNA, 372 bp, human
caccaagacgactgcttcagcttcttctcttatccttactttctttaatagatatttattaaactgtccagtgaaaaggtgccacaatgcccagtattgtaaacaacaggtttgcattcatgaagctttcattcattctggagtctactaatttacctgaatggtgtttgcattctgtgaaatgcctctccacgttgcatatgtcacacttttgtctgcacataactcttttttcacaagaagggtcactgccacaacagcacagtcagcgggtgaattacaggtgcctgctgcctgcctacctgggtaatctgatcttgtctgtatcgccgtgtgctcatcactgaagaattgcaggccactcatgtcagt

SEQ ID NO: 108 - DNA, 475 bp, human
ctcgctcagccagatgcaatcaatgccccagtcacctgctgctataacttcaccaataggaagatctcagtgcagaggctcgcgagctatagaagaatcaccagcagcaagtgtcccaaagaagctgtgatcttcaagaccattgtggccaaggagatctgtgctgaccccaagcagaagtgggttcaggattccatggaccacctggacaagcaaacccaaactccgaagacttgaacactcactccacaacccaagaatctgcagctaacttattttcccctagctttccccagacatcctgttttattttattataatgaattttgtttgttgatgtgaaacattatgccttaagtaatgttaattcttatttaagttattgatgttttaagtttatctttcatggtactagtgttttttagatacagagacttggggaaattgcttttcctcttgaaccacagttctaccc

SEQ ID NO: 109 - DNA, 504 bp, human
ttctttgtgcggattctgaatgccaatgatgaggccacagtgtctgttcttggggagcttgcagcagaaatgaatggggtttttgacactacattccaaagtcacctgaacaaagccttatggaaggtagggaagttaactagtcctggggctttgctctttcagtgagctaggcaatcaagtctcacagattgctgcctcagagcaatggttgtattgtggaacactgaaactgtatgtgctgtaatttaatttaggacacatttagatgcactaccattgctgttctactttttggtacaggtatattttgacgtcactgatattttttatacagtgatatacttactcatggccttgtctaacttttgtgaagaactattttattctaaacagactcattacaaatggttaccttgttatttaacccatttgtctctacttttccctgtacttttcccatttgtaatttgtaaaatgttctcttatgatcaccatgtattt

SEQ ID NO: 110 - DNA, 321 bp, human
gtttggatggtggaaggtctcattttattgagatttttaagatacatgcaaaggtttggaaatagaacctctaggcaccctcctcagtgtgggtgggctgagagttaaagacagtgtggctgcagtagcatagaggcgcctagaaattccacttgcaccgtagggcatgctgataccatcccaatagctgttgcccattgacctctagtggtgagtttctagaatactggtccattcatgagatattcaagattcaagagtattctcacttctgggttatcagcataaactggaatgtagtgtcagaggatactgtggctt

SEQ ID NO: 111 - DNA, 251 bp, human
tttccctctaagtttacactaacatctacccaagatagactgttttttaacagtcaatttcagttcagctaacatatattaatacctttgtaactctttgctatggcttttgttatcacaccaaaactatgcaattggtacatggttgtttaagaagaaaccgtatttttccatgataaatcactgtttgaaatatttggttcatggtatgatcgaaatgtaaaagcataattaacacattggctgctagt

SEQ ID NO: 112 - DNA, 432 bp, human
gaagatgatttatctgctggcttggcactgattgacctgggtcagagtatagatatgaaactttttccaaaaggaactatattcacagcaaagtgtgaaacatctggttttcagtgtgttgagatgctcagcaacaaaccatggaactaccagatcgattactttggggttgctgcaacagtatattgcatgctctttggcacttacatgaaagtgaaaaatgaaggaggagagtgtaagcctgaaggtctttttagaaggcttcctcatttggatatgtggaatgaattttttcatgttatgttgaatattccagattgtcatcatcttccatctttggatttgttaaggcaaaagctgaagaaagtatttcaacaacactatactaacaagattagggccctacgtaataggctaattgtactgctctta

SEQ ID NO: 113 - DNA, 388 bp, human
ttgctggcttctcttatactaacccagagtttgtcattaatgtgtaggtgaatgcaaactccatcgttgagcctggggtgtaagacttcaagccaagcgtatgtatcaattctagtcttccaggattcacggtgcacatgctggcattcaacatgtggaaagcttgtcttagaggcctttcttgtatgtgtagcttgctagtttgttttctacatttgaaaatgtttagtttagaataagcgcattatccaattatagaggtacaattttccaaacttccagaaactcatcaaatgaacagacaatgtcaaaactactgtgtctgataccaaaatgcttcagtatttgtaatttttcaagtcagaagctgatgttcctggtaaaagtt

SEQ ID NO: 114 - DNA, 471 bp, human
gctgatggagatcgtcacctacggccggatcccttacccagggatgtcaaaccctgaagtgatccgagctctggagcgtggataccggatgcctcgcccagagaactgcccagaggagctctacaacatcatgatgcgctgctggaaaaaccgtccggaggagcggccgaccttcgaatacatccagagtgtgctggatgacttctacacggccacagagagccagtaccaacagcagccatgatagggaggaccagggcagggcagggggtgcccaggtggtggctcgaaggtggctccagcaccatccgccagggcccacacccccttcctactcccagacacccaccctcgcttcagccacagtttcctcatctgtccagtgggtaggttggactggaaaatctctttttgactcttgcaatccacaatctgacattctcaggaagcccccaagttgatatttctatt

SEQ ID NO: 115 - DNA, 514 bp, human
aacttactgcgatttgttctcaaaacttaaaaataagcaagcaaatggctgatactaccaagagaactggaagatggataccacacaaacttcttgtataaaaatatgaatgctgaaatgtttcagacatttttaatttaataaacctgtaaccacatttaagtgatctaaaacccatagcattgtagtcatggcaacccgctaaactttctcatgcaactaaaatttctgggggaaatgagggtgggggttgtacatttcccattgtaaaataagtgttttaaatgtcctgtactgctaacgaatgactttctatatgtccaggagttctccagtggaataactatgcactactttacatttcatggggatgcacaaaaacaaaaaagtattacatttttagttgctgtttgtaccaaccttaaattacatatgtttaacaacaacaaatcaaaaatcctatttctattgagtttttaatactgactagcaactctgaagtcttaattccttt

SEQ ID NO: 116 - DNA, 363 bp, human
gattgctgacttcggattggcccgattgatagaagacaatgagtacacagcaagacaaggtgcaaagttccccatcaagtggacggcccccgagcgagccctgtacgggaggttcacaatcaagtctgacgtgtggtcttttggaatcttactcacagagctggtcaccaaaggaagagtgccatacccaggcatgaacaaccgggaggtgctggagcaggtggagcgaggctacaggatgccctgcccgcaggactgccccatctctctgcatgagctcatgatccactgctggaaaaaggaccctgaagaacgccccacttttgagtacttgcagagcttcctggaagactactttaccgc

SEQ ID NO: 117 - DNA, 350 bp, human
tatgaaacataccccctattttacagagctcttttttcccctcataaccttgtttttggtagaaaataagctagagaaattaagccatcgtgttggtgagtgttcctaggctaatgataatctgtataattcacatcctgaaactaaggaatacagggttgaaaaaatattaatatgtttgtcagaaggaaaaataatgcatttattttcccccccaccccccgccccatggaatatttaatctatttaatcttcttgcatttatttctcaagaattactggctttaaaagaagccaaagcactactagctttttttccatattggtatttttgatgctgcttccaattt

SEQ ID NO: 118 - DNA, 485 bp, human
tctctgtttcttcaatggttctcttcccttttccatcctccaaacctggcctgagcctcctgaagttgctgctgtgaatctgaaagacttgaaaagcctccgcctgctgtgtggacttcatctcaaggggcccagcctcctctggactccaccttggacctcagtgactcagaacttctgcctctaagctgctctaaagtccagactatggatgtgttctctaggccttcaggactctagaatgtccatatttatttttatgttcttggctttgtgttttaggaaaagtgaatcttgctgttttcaataatgtgaatgctatgttctgggaaaatccactatgacatctaagttttgtgtacagagagatatttttgcaactatttccacctcctcccacaaccccccacactccactccacactcttgagtctctttacctaatggtctctacctaatggacctccgtggccaaaaagtaccat

SEQ ID NO: 119 - DNA, 382 bp, human
  misc_feature (57)..(57): n is a, c, g, or t
aagctatatctattctaaaccttatttagacattggtaccagttacccaggtgaaanatatggagtaactttgttttgtatggtaaggtttaggaatggtggatgaagggtatctctatataaataaagtgctcaacaatgtgcaatgattgtaaatttagtaagatattacagccatttcatgaatgctttaccattcaacatagtatctattacaaaacacctttcttgtatccatatacttcaggtgttgctgttaacatttactatgatatttattttaaccaaaatgttactcacattaaatgtttattctttaaaatgaatgtattatgtttttaacccacaaatgcatacttaccctgtgcctcatatttcaata

SEQ ID NO: 120 - DNA, 563 bp, human
gaggacatcaatgaccagatccagaacgccgccttcatgtacccggccagcccctggagccacatctcagctggagccattgacctcatcaacaacctgctgcaggtgaagatgcgcaaacgctacagcgtggacaaatctctcagccacccctggttacaggagtaccagacgtggctggacctccgagagctggaggggaagatgggagagcgatacatcacgcatgagagtgacgacgcgcgctgggagcagtttgcagcagagcatccgctgcctgggtctgggctgcccacggacagggatctcggtggggcctgtccaccacaggaccacgacatgcaggggctggcggagcgcatcagtgttctctgaggtcctgtgccctcgtccagctgctgccctccacagcggttcttcacaggatcccagcaatgaactgttctagggaaagtggcttcctgcccaaactggatgggacacgtggggagtggggtggggggagctatttccaaggcccctccctgtttccccagcaattaaaacggactcatctctggccc

SEQ ID NO: 121 - DNA, 562 bp, human
tcattgggtactcctgaaatcagacatgttcctgtagaaagaattttaagttaggctttctatgcacctatcaagaatcaagagaatagattgtatcaaacaacggcagggaaatccttcagcaattctaatccactttgggttttcagctgtttttacatctaaagcaatagactagaactgaattatcttctacatagtaaaatcacaattgtggaattctggtgatattaaggtgaaataacaaaacacaaaaggccctattttaacagttgatgtgacagtaagttttaatagaacctgtaacttcattttggaaatgcttctccaccaaataagggctttttcccctatttaaggagccagatggattgaaagatgtggaaataggcagctgtagatcttgatcttccaggtaccccatgtacctttattgagcttaattataatactgtcaaattgccacgatctcactaaaggatttctatttgctgtcagttaaaaataaagccctaaatacatttttattctttctactgagggcattgtctgttttctttgt

SEQ ID NO: 122 - DNA, 501 bp, human
gtgtccgaagttgagatggcctgccctactggcaaagaggtgacaggaaggctgggagcagctttgttaaattgtgttcagttctgttacacagtgcattgccctttgttgggggtatgcatgtatgaacacacatgcttgtcggaacgctttctcggcgtttgtcccttggctctcatctcccccattcctgtgcctactttgcctgagttcttctacccccgcagttgccagccagattgggagtctgtttgttccaatgggttgagctgtctttgtcgtggagatctggaactttgcacatgtcactactggggaggtgttcctgctctagcttccacgatgaggcgccctctttacctatcctctcaatcactactcttcttgaagcactattatttattcttccgctgtctgcctgcagcagtactactgtcaacatagtgtaaatggttctcaaaagcttaccagtgtggacttggtgttagccacgctgtttac

SEQ ID NO: 123 - DNA, 506 bp, human
aatcagcatctttccaatgaggtcaaaacttggaaggaaagaacccttaaaagagaggctcacaaacaagtaacttgtgagaattctccaaagtctcctaaagtgactggaacagcttctaaaaagaaacaaattacaccctctcaatgcaaggaacggaatttacaagatcctgtgccaaaggaatcaccaaaatcttgtttttttgatagccgatcaaagtctttaccatcacctcatccagttcgctattttgataactcaagtttaggcctttgtccagaggtgcaaaatgcaggagcagagagtgtggattctcagccaggtccttggcacgcctcctcaggcaaggatgtgcctgagtgcaaaactcagtagactcctctttgtcacttctctggagatccagcattccttatttggaaatgactttgtttatgtgtctatccctggtaatgatgttgtagtgcagcttaatttcaattcagtctttactttgccactag

SEQ ID NO: 124 - DNA, 515 bp, human
  misc_feature (27)..(28): n is a, c, g, or t
gcttgtacgtacatatgtgaactcatnnggaaatattgtgtgtttaatgcaaatgatatattgaattgtttagcaattngttttctttgcttaacgatgtttttgagatctgtgcatgttacttaatgtagctcaatccatcttctgtaattgctgtatagattgtcatcatatgattaccacattttacttacgcatttcttttgtgatggacattaagactgtttttaggttttgctattacaaaatactacacaggagcatcactatgcctgtgtgaaagtatatgtatgaaagtttacctagggttgattcctagaagtggaattgcaaagtcataggatatttatatattggtttttaataatacttccaaattgccctcctgtactatttactcagtatttttcttgaggttgatctgaggtctaacattgttatcctatatcattttcatcccaagtagtgatatctgtgaaatcacaggtttgatgtgtgctaattatgtattcttc

SEQ ID NO: 125 - DNA, 468 bp, human
cactctgatagccagctgttccaatgaccagactgtgcgtgtatgggtcgtagcaacaaaggaatgcaaggctgagctccgagagcatgagcatgtggtagaatgcatttcctgggctccagaaagctcatattcctccatctctgaagcaacaggatctgagactaaaaaaagtggtaaacctgggccattcttgctgtctggatccagagacaagactattaagatgtgggatgtcagtactggcatgtgccttatgaccctcgtgggtcatgataactgggtacgtggagttctgttccattctggggggaagtttattttgagttgtgctgatgacaagaccctacgcgtatgggattacaagaacaagcgatgcatgaagaccctcaatgcgcatgaacactttgttacctccttggatttccacaagacggcaccctatgtcgtcactggcagcgtagatca

SEQ ID NO: 126 - DNA, 423 bp, human
atttgtgactctttcctaaagccaaagtttctgttgaattatgttttgacacacccctaagtacaaggtggtatggttgtatacacatgctgccttcttggggattcaaaaacaggtttttgattttgaatagcaattagtgatatagtgctgtttaagctactaacgataaaaggtaataacattttatacaatttccatatagtctattcattaagtaatctttttacagttgcatcaggcctgaacccgtccattcagaaagcttcaaattatagaaacaatactgttctatacgagtgaccgattatgctttctttggcctacattctttattctgcggtgaagttgaggcttataagttaaaacaaaggaactaacttactgtccaccagtttatacagaactcacagtacctatgac

SEQ ID NO: 127 - DNA, 482 bp, human
ggaatcttggcgttcagcagagtcagggatgggtccattatatgatccatgaaccagaacctcacatcttgctgttccggcgcccactacccaagaaaccaaagaaatgaagctggcaagctacttttcagcctcaagctttacacagctgtccttacttcctaacatctttctgataacattattatgttgccttcttgtttctcactttgatatttaaaagatgttcaatacactgtttgaatgtgctggtaactgctttgcttcttgagtagagccaccaccaccatagcccagccagatgagtgctctgtggacccacagcctaagctgagtgtgaccccagaagccacgatgtgctctgtatccagaacacacttggcagatggaggaagcatctgagtttgagaccatggctgttacagggatcatgtaaacttgctgtttttgttttttctgccgggtgttgtatgtgtggtgac

SEQ ID NO: 128 - DNA, 496 bp, human
cgctctcgtttcattttctgcagcgcgccacgaggatggcccacaagcagatctactactcggacaagtacttcgacgaacactacgagtaccggcatgttatgttacccagagaactttccaaacaagtacctaaaactcatctgatgtctgaagaggagtggaggagacttggtgtccaacagagtctaggctgggttcattacatgattcatgagccagaaccacatattcttctctttagacgacctcttccaaaagatcaacaaaaatgaagtttatctggggatcgtcaaatctttttcaaatttaatgtatatgtgtatataaggtagtattcagtgaatacttgagaaatgtacaaatctttcatccatacctgtgcatgagctgtattcttcacagcaacagagctcagttaaatgcaactgcaagtaggttactgtaagatgtttaagataaaagttcttccagtcagtttttctcttaagtgcct

SEQ ID NO: 129 - DNA, 302 bp, human
ttgtgtgagctattcaaactcttcaacccctgaacagggtattaagcttccaaaataatgatggggataaatatggaaatcctttttaagttgtatttccattaaacaaaaacccttataattcatactatcatgaatttgctttatccatctcatttgcataacagttcatctgtctggtcccattaggctctaccaaagaaagactctgatgagtggacattattactgtgactcttgtaagtagccataaataaaccaaaatagtatcaaatttaggtatgaaattccacatgtgcaaa

SEQ ID NO: 130 - DNA, 206 bp, human
atttacaggttcttaggctccatcctgtttgtatgaaattataatctgtggattggcctttaagcctgcattcttaacaaactcttcagttaattcttagatacactaaaaatctgagaaactctacatgtaactatttcttcagagtttgtcatatactgcttgtcatctgcatgtctactcagcatttgattaacatttgtgta

SEQ ID NO: 131 - DNA, 279 bp, human
ggttgaagagcaatgtgtggctgaagacttttgttatgaggagctgcagattagctaggggacagctggaattatgctggcttctgataattattttaaaggggtctgaaatttgtgatggaatcagattttaacagctctcttcaatgacatagaaagttcatggaactcatgtttttaaagggctatgtaaatatatgaacattagaaaaatagcaacttgtgttacaaaaatacaaacacatgttaggaaggtactgtcatgggctaggcatggtg

SEQ ID NO: 132 - DNA, 485 bp, human
gggagtttcctgagggttttctcataaatgagggctgcacattgcctgttctgcttcgaagtattcaataccgctcagtattttaaatgaagtgattctaagatttggtttgggatcaataggaaagcatatgcagccaaccaagatgcaaatgttttgaaatgatatgaccaaaattttaagtaggaaagtcacccaaacacttctgctttcacttaagtgtctggcccgcaatactgtaggaacaagcatgatcttgttactgtgatattttaaatatccacagtactcactttttccaaatgatcctagtaattgcctagaaatatctttctcttacctgttatttatcaatttttcccagtatttttatacggaaaaaattgtattgaaaacacttagtatgcagttgataagaggaatttggtataattatggtgggtgattattttttatactgtatgtgccaaagctttactactgtg

SEQ ID NO: 133 - DNA, 316 bp, human
gggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagctcgctctgtccgtgcccagcgccacaccgacatgcccaagacccagaaggaagtacatttgaagaacgcaagtagagggagtgcaggaaacaagaactacaggatgtaggaagaccctcctgagga

SEQ ID NO: 134 - DNA, 551 bp, human
agccctttgctctggaggaagttctccagcttcagctcaactcacagcttctccaagcatcaccctgggagtttcctgagggttttctcataaatgagggctgcacattgcctgttctgcttcgaagtattcaataccgctcagtattttaaatgaagtgattctaagatttggtttgggatcaataggaaagcatatgcagccaaccaagatgcaaatgttttgaaatgatatgaccaaaattttaagtaggaaagtcacccaaacacttctgctttcacttaagtgtctggcccgcaatactgtaggaacaagcatgatcttgttactgtgatattttaaatatccacagtactcactttttccaaatgatcctagtaattgcctagaaatatctttctcttacctgttatttatcaatttttcccagtatttttatacggaaaaaattgtattgaaaacacttagtatgcagttgataagaggaatttggtataattatggtgggtgattattttttatactgtatgtgccaaagctttactactgtg

SEQ ID NO: 135 - DNA, 162 bp, human
gttgttcttagacaagtgcctcctggtgcctgcggcatccttctgcctgtttctgtaagcaaatgccacaggccacctatagctacatactcctggcattgcactttttaaccttgctgacatccaaatagaagataggactatctaagccctaggtttctt

SEQ ID NO: 136 - DNA, 527 bp, human
aagacctacaatggaaccaaccctgatgctgctagccgggctattgattatgtacagagacagctgcattgttgtggaattcacaactactcagactgggaaaatacagattggttcaaagaaaccaaaaaccagagtgtccctcttagctgctgcagagagactgccagcaattgtaatggcagcctggcccacccttccgacctctatgctgaggggtgtgaggctctagttgtgaagaagctacaagaaatcatgatgcatgtgatctgggccgcactggcatttgcagctattcagctgctgggcatgctgtgtgcttgcatcgtgttgtgcagaaggagtagagatcctgcttacgagctcctcatcactggcggaacctatgcatagttgacaactcttgcctgagctttttggtcttgttctgatttggaaggtgaattgagcaggtctgctgctgttggcctctggagttcatttagttaaagcacatgtacactggtgttggacagagcagcttggct

SEQ ID NO: 137 - DNA, 441 bp, human
aacattatccagaatcctacagaccagcagaagaaggaccatgaaaaagctgaatttgaggtacatgaagtatatgctgcggatgttctcgtcagctcaggagagggcaaggccaaggatgcaggacagagaaccactatttagaaacgagactcctctaaacagtatggactgaagaggaaaacttcacgtgccttcttcagtgaggtggaaaggcattttgatgccatgccgtttactttaagagcatttgaagatgagaagaaggctccgatggatgtggtggagtgcaccaaacatagactgctgcaaccgtttaatgttctctatgagaaggagggcgaatttgttgcccagtttaaatttacaattctgctcatgcccaatggccccatgcagaaaaccagtggtcccttcaagcctgacctctacaggtctgag

SEQ ID NO: 138 - DNA, 480 bp, human
  misc_feature (207)..(208): n is a, c, g, or t
tacttaggtcaaatttctgttctctcttccccaaataatattaaagtattatttgaactttttaagatgaggcagttcccctgaaaaagttaatgcagctctccatcagaatccactcttctagggatatgaaaatctcttaacacccaccctacatacacagacacacacacacacacacacacacacacacacacacacacacannttcaccctaaggatccaatggaatactgaaaagaaatcacttccttgaaaattttattaaaaaacaaacaaacaaacaaaaagcctgtccacccttgagaatccttcctctccttggaacgtcaatgtttgtgtagatgaaaccatctcatgctctgtggctccagggtttctgttactattttatgcacttgggagaaggcttagaataaaagatgtagcacattttgctttcccatttattgtttggccagctatgccaatgtggtgcta

SEQ ID NO: 139 - DNA, 514 bp, human
tatctctccatgttcagttccaaggagtcccagcggggcatgggctacatgcccaaacgtggcctggaggtgaacaagtgtgagatcgccaggttctacaagctgcacgagcggaggtgtgagcccattgccatgacagtgcctcgaaagtcggacctgttccaggaggacctgtacccacccaccgcagggcccgaccctgccctcacggctgaggagtggctggggggtcgggatgctgggcccctcctcatctccctcaaggatggctacgtacccccaaagagccgggagctgagggtcaaccggggcctggacaccgggcgcaggagggcagcaccagaggccagtggcactcccagctcggatgccgtgtctcggctggaggaggagatgcggaagctccaggccacggtgcaggagctccagaagcgcttggacaggctggaggagacagtccaggccaagtagagccccgcagggcctccagcagggtcagacattcacacccatc

SEQ ID NO: 140 - DNA, 453 bp, human
acctaacgaagtatccttcagcctgaaagaggaatgaagtactcatacatgttacaacacggacgaaccttgaaaactttatgctaagtgaaataagccagacatcaacagataaatagtttatgattccacctacatgaggtactgagagtgaacaaatttacagagacagaaagcagaacagtgattaccagggactgaggggaggggagcatgggaagtgacggtttaatgggcacagggtttatgtttaggatgttgaaaaagttctgcagataaacagtagtgatagttgtaccgcaatgtgacttaatgccactaaattgacacttaaaaatggtttaaatggtcaattttgttatgtatattttatatcaatttaaaaaaaaacctgagccccaaaaggtattttaatcaccaaggctgattaaaccaaggctagaaccacctgcc

SEQ ID NO: 141 - DNA, 454 bp, human
actggtctatctctatcctgacattcccaaggaggaggcattcggaaagtattgtcggccagagagccaggagcatcctgaagctgacccaggcgctgccccatacctgaagaccaagtttatctgtgtgacaccaacgacctgcagcaataccattgacctgccgatgtccccccgcactttagattcattgatgcagtttggaaataatggtgaaggtgctgaaccctcagcaggagggcagtttgagtccctcacctttgacatggagttgacctcggagtgcgctacctcccccatgtgaggagctgagaacggaagctgcagaaagatacgactgaggcgcctacctgcattctgccacccctcacacagccaaaccccagatcatctgaaactactaactttgtggttccagattttttttaatctcctacttctgctatctttgagc

SEQ ID NO: 142 - DNA, 112 bp, human
ggaactaagagctagtatcttggattaactgatgcctgctagtgctttctgattactcgcattctgtttcttgctttaaaagaagagtaaagacaagagtgttggaccagta

SEQ ID NO: 143 - DNA, 524 bp, human
gtctctttgctatatgaccttgaaaatcttccggcatccaaggattccattgtgcatcaagctggcatgttgaagcgaaattgttttgcctctgtctttgaaaaatacttccaattccaagaagagggcaaggaaggagagaacagggcagttatccattatagggatgatgagaccatgtatgttgagtctaaaaaggacagagtcacagtagtcttcagcacagtgtttaaggatgacgacgatgtggtcattggaaaggtgttcatgcaggagttcaaagaaggacgcagagccagccacacagccccacaggtcctctttagccacagggaacctcctctggagctgaaagacacagacgccgctgtgggtgacaacattggctacattaccttttgtgctgttcccttcgtacaccaatgccagtgctcgagacacaccattaaccttgttccacacgttccgggactacctcccctaccacattaagggcctttaaggcctatattcacacacgtatg

SEQ ID NO: 144 - DNA, 483 bp, human
ggacagtgtttcaacaagcctaggctatctcgtaagttgaaaaatatcccactatagttgcttcatgagtatgaagtaagatggcctctgatttacactggttcaatttacaaattttcaactttatgataggtttatccgggtactaaatgcatttcaacttgatagtttcaacttatgataggtttaccaggatgtagtcccactgttgaggagcatctatttaggggttaattactttagtaataagtggaaagtaagataccttgagtaatgtttgcctataaaattgtcagcgtatttttacactattggctcaagaatgttataatgctaagggacataagttggcaaccacttggtttttggaaggactttcggtattgtattagaagtctgccctagctgttaaatttctgggtatttatcctaaggaattaattaaagagttaattgttcctttcttcagtgggccattgtttt

SEQ ID NO: 145 - DNA, 499 bp, human
  misc_feature (116)..(116): n is a, c, g, or t
aatattgttttgtgtgactgaatgtgttagctcatgcctgtaattccagcactttgggaggctgaggcaggaggatcacttgaggctgggagttcaagaccagcctgggcaacatngcnagacccnntntntannaaaaaaggnattatgnggtgnanttantccaatgtgnggnantntntngntgnganncctgntcntcccgttgatcaccagggttgaatttattaaattaggcatattaattcattgaagttgactttttagtatcctgctttaagttgggagttatgagttgcagaaacgaattgctgaaatggaaactcaaaaggaaaaaattcatgaagataccaaagaaattaatgagaagagcaatatactatcaaatgaaatgaaagctaagaataaagatgtaaaagatacagaaaagaaactgaataaaattacaaaatttattgaggagaataaagaaaaatttacacagctagatttggaagat

SEQ ID NO: 146 - DNA, 353 bp, human
aattcgagtctatacaaaagccttgagttctttagaactcagtagccatcttgcaaaagatcttctggttctattgaatgagattctggagcaagtaaaagataggacatgtctgagagctttggagaaaatcaagattcagttagaaaaaggaaataaagaatttggtgaccaagctgaagcagcacaggatgccaccttgactacaactactttccaaaatgaagatgaaaagaataaagaagtatatatgactccactcaggggtgtaaaagcaacccaagcatcaaagtctactcagctaaagactaacagaggacagagaaaagtgacagtttcagctaggacgaaca

SEQ ID NO: 147 - DNA, 479 bp, human
aaatgatacttactgaactgtgtgtaattgttcctttatttttttggtacctatttgacttaccatggagttaacatcatgaatttattgcacattgttcaaaaggaaccaggaggtttttttgtcaacattgtgatgtatattcctttgaagatagtaactgtagatggaaaaacttgtgctataaagctagatgctttcctaaatcagatgttttggtcaagtagtttgactcagtataggtagggagatatttaagtataaaatacaacaaaggaagtctaaatattcagaatctttgttaaggtcctgaaagtaactcataatctataaacaatgaaatattgctgtatagctccttttgaccttcatttcatgtatagttttccctattgaatcagtttccaattatttgactttaatttatgtaacttgaacctatgaagcaatggatatttgtactgtttaatgttctgtga

SEQ ID NO: 148 - DNA, 534 bp, human
  misc_feature (235)..(253): n is a, c, g, or t
gctccaggccaaagtgccggtgagacccatggctgtgttggtgtggcccatggctgttggtgggacccgtggctgatggtgtggcctgtggctgtcggtgggactcgtggctgtcaatgggacctgtggctgtcggtgggacctacggtggtcggtgggaccctggttattgatgtggccctggctgccggcacggcccgtggctgttgacgcacctgtggttgttagtggggcnnnnnnnnnnnnnnnnnnnccaaggccggcaggtcaacctcgcgcttgctggccagtccaccctgcctgccgtctgtgcttcctcctgcccagaacgccnnnnnnnnnnnnnnnnnnnnnnnnnnnnnnnnngtgcccttcctgcnnnnnnnnnnnnnnntcctgggacggcggcagtattgaagctcgtgacaagtgccttcacacagacccctcgcaactgtccacgcgtgccgtggcaccangcgctgcccancctgcnggccccggccgcccctcctcgtgaaagtgcatttttgt

SEQ ID NO: 149 - DNA, 530 bp, human
ggattatccatcttgctaatggcctattatttatttcactttttgttggtctttatatccttttattaatgtgctaaaggaacctgtatatctattttggaactctttgaatagtctaaaatagactaaaaatggagtattttatagtttaaattacaaaccaaggtggttccccctaagatatatatcttggtttaccatgattccaaacaaaactaacttgtttaaaaatatttggagtaaagttttatttgcattacaaataggatacaaaaatagttgaatcaggatacagaaatctgctgtgttttgactagttatccctgttttatttttttagatgtgcttaatttatggtgtaactaaagattttgtttgtgtaatgcatgattaagacaataaagtattttttctagtcttccgagaaacttttctgatcagtttgcgagttttgatgagttttgtaaggtttttgttttacaaactatgaatcagcaaatttttaagattgtaccacatagcaaacatac

SEQ ID NO: 150 - DNA, 220 bp, human
ggacgaacaggaggtgtcagactgctgaagccgactctgaaagtgatcatgaagttccagaaccagaatcagaaatgaagatgagactaccaagacgagccaaaaccgcagcactagaaaaaagtaaacttaaccttgcccaatttctcaatgaagatctaagttaggaaagacgatggaggtggaatcctttaagattatgtccagttatttgctttaa

SEQ ID NO: 151 - DNA, 401 bp, human
ggacgccgaactcgagcttgtagacagcttggatcctttctctgaaggaatcatgttcagtgttcgaccacctaagaaaagttggaaaaagatcttcaacctttcgggaggagagaaaacacttagttcattggctttagtatttgctcttcaccactacaagcccactcccctttacttcatggatgagattgatgcagcccttgattttaaaaatgtgtccattgttgcattttatatatatgaacaaacaaaaaatgcacagttcataataatttctcttcgaaataatatgtttgagatttcggatagacttattggaatttacaagacatacaacataacaaaaagtgttgctgtaaatccaaaagaaattgcatctaagggactttgttgaactt

SEQ ID NO: 152 - DNA, 445 bp, human
tgtccctaacagtccacaggcaaacaaatggtacagtcataagagccatctgtcacggacccacgcccagaggaacgtgcagaaaaaagcagagctacatggctgtgggcaactataagccaaatatttggctcagaacaggtgtccatgggacaaaaaagaacgatcctccacttgaccaagaaaaaagtgattctcccagaagcacaaagcatactcttgcccctcaggtgttgcttgtgtacatcgtacccatccattcggcttcacctgcagccaacggcctggaatcgcaaagagacaccactctgggcagagcagagcagggtatggggtggggagagagggtggagggttttataaacaaacttaacagcaatattgaaaggaggtgggggattgagggagggacagagtgttggagggccagagactagtcctgaga

SEQ ID NO: 153 - DNA, 499 bp, human
aagtcgtctatttctttctagttgcatcaacttgcacactggtgtctaactatatgtggtattcggaacttgtaattcttattgagcagccattgcctatattttctcagctcctcactgatttctttatatgagctcagctgacaagcaaatgcagctattttgtttaccctctcttcataccggaccatcaacaacagtgttctaactcatccctgatggtctgtgagatgtagactattacacaagattcaagttactcgtaagcaacttttagatagagttggtcctgttaggaggagactcttgatgtcaccttcagtatcttgaaagcgggtcccctccccgaggctcttaattctttgaaaacttgatgctgtttcagctgaaaaattagcaagactgttaaaaaaaaaaagttgaggggagctgtttaagaaactgaaaagtaattgcaaactacattggataattgtgactttcagttgttgttctgtat

SEQ ID NO: 154 - DNA, 288 bp, human
tgccagccaggctagagggatgctcatgcaggttgcaccccagtcctggattagccctcttgatggatgaagacactgaggactcagagaggctgagtcacttacctgaggacacccagccaggcagagctgggattgaaggacccctatagagaagggcttggcccccatggggaagacacggatggaaggtggagcaaaggaaaatacatgaaattgagagtggcagctgcctgccaaaatctgttccgctgtaacagaactgaatttggaccccagcacagtggc

SEQ ID NO: 155 - DNA, 447 bp, human
ggagccaatccatgcagatattttgttggaaacttataagaggaagattgctgatgaaggaagactttttctggctgaatttcagagcatcccgcgggtgttcagcaagtttcctataaaggaagctcgaaagccctttaaccagaataaaaaccgttatgttgacattcttccttatgattataaccgtgttgaactctctgagataaacggagatgcagggtcaaactacataaatgccagctatattgatggtttcaaagaacccaggaaatacattgctgcacaaggtcccagggatgaaactgttgatgatttctggaggatgatttgggaacagaaagccacagttattgtcatggtcactcgatgtgaagaaggaaacaggaacaagtgtgcagaatactggccgtcaatggaagagggcactcgggcttttggagatgt

SEQ ID NO: 156 - DNA, 404 bp, human
aagcaccctgtatcacagactggcaacaagaagatggtaccgtgcatcgcacctatttaagagggaacttagcagagagcaaatgctatttgataacagttactccagtatatgctgatggaccaggaagccctgaatccataaaggcataccttaaacaagctccaccttccaaaggacctactgttcggacaaaaaaagtagggaaaaacgaagctgtcttagagtgggaccaacttcctgttgatgttcagaatggatttatcagaaattatactatattttatagaaccatcattggaaatgaaactgctgtgaatgtggattcttcccacacagaatatacattgtcctctttgactagtgacacattgtacatggtacgaatggcagcatacacagat

SEQ ID NO: 157 - DNA, 485 bp, human
  misc_feature (407)..(407): n is a, c, g, or t
ggttcaccttggcatcaatttgccctgaaacttagctgtgctgggattattctccttgtcttggttgttactgggttgagtgtttcagtgacatccttaatacagaaatcatcaatagaaaaatgcagtgtggacattcaacagagcaggaataaaacaacagagagaccgggtctcttaaactgcccaatatattggcagcaactccgagagaaatgcttgttattttctcacactgtcaacccttggaataacagtctagctgattgttccaccaaagaatccagcctgctgcttattcgagataaggatgaattgatacacacacagaacctgatacgtgacaaagcaattctgttttggattggattaaatttttcattatcagaaaagaactggaagtgganaaacggctcttttttaaattctaatgacttagaaattagaggtgatgctaaagaaaacagctgtatttccatctcaca

SEQ ID NO: 158 - DNA, 230 bp, human
tcccttgcatccaggtgcagcatggactgagttcttgacaacagaatgtggtcagaagtgacatatgccaacacggggtctgggtgggggctcccccacatcctttccttgcctatgagctggaacataacacatgcctatgatccagctttggtcatacccaaggggaaggtggagcaagaaatgaaaaggaacctgaatccctgaatgactgcatggatagaaccact

SEQ ID NO: 159 - DNA, 403 bp, human
ttattcttctaattaacagctcctaggaaaatgtagacttttgctttatgatattctatctgtagtatgaggcatggaatagttttgtatcgggaatttctcagagctgagtaaaatgaaggaaaagcatgttatgtgtttttaaggaaaatgtgcacacatatacatgtaggagtgtttatctttctcttacaatctgttttagacatctttgcttatgaaacctgtacatatgtgtgtgtgggtatgtgtttatttccagtgagggctgcaggcttcctagaggtgtgctataccatgcgtctgtcgttgtgcttttttctgtttttagaccaattttttacagttctttggtaagcattgtcgtatctggtgatggattaacatatagcctttgttttct

SEQ ID NO: 160 - DNA, 542 bp, human
gactttgagaacccagtcccatacactggggcagttaaggttggagccatccagcgctggctgaaggggcaaggggtctacctaggtatgcctggttgcctgcctgtatacgacgccctggccggggagttcatcagggcctctggtgtggaggcccgccaggccctcttgaagcaggggcaagataacctctcaagtgtgaaggagactcagaagaagtgggccgagcaatacctgaagatcatggggaagatcttagaccaaggggaggacttcccagcatcagagatgacacggatcgccaggctgattgagaagaacaagatgagtgacggcaagaaggaggagctccagaagagcttaaacatcctgactgccttccagaagaagggggccgagaaagaggagctgtaaaaaggctgtctgtgattttccagggtttggtgggggtagggaggggagagttaacctgctggctgtgagtcccttgtggaatataagggggtagtgggaacagtggtactaacccacgattctgagcc

SEQ ID NO: 161 - DNA, 420 bp, human
cattggatttggttctgcacttcttgaagaagttgatcctaatcctgcgaatttcgtgggagctggaatcatccacacgaaaaccacccagattggatgcctgctgcgcttggagccgaacctgcaagcccagatgtaccggctcacgctgcgcacaagtaaggaagccgtttctcagagattatgtgaattgctctcagcgcagttttagtcctgaggatggaagaccaggcttgtgtgtcttgtgttgtcttcgtctgtgccgtttgtcttcgtggccatcctgcagatgagcaccgtgtccagtgccacagcacaaggcgcctccccgccccgccgccccacacctctcccctttgggctggacgggaacacacgtgtgtggctcaggaggaaaagctcagcctggactgtggcagc

SEQ ID NO: 162 - DNA, 420 bp, human
  misc_feature (280)..(280): n is a, c, g, or t
cattggatttggttctgcacttcttgaagaagttgatcctaatcctgcgaatttcgtgggagctggaatcatccacacgaaaaccacccagattggatgcctgctgcgcttggagccgaacctgcaagcccagatgtaccggctcacgctgcgcacaagtaaggaagccgtttctcagagattatgtgaattgctctcagcgcagttttagtcctgaggatggaagaccaggctcgtgtgtcttgtgttgtcttcgtctgtgccgtttgtcttcgtggcnatcctgcagatgagcaccgtgtccagtgccacagcacaaggcgcctccccgccccnccgccccacacctctcccctttgggctggacgggaacacacgtgtgtggctcaggaggaaaagctcagcctggactgtggcagc

SEQ ID NO: 163 - DNA, 192 bp, human
ataaatttgttgtgtactacgtttggtattttgtcttattgtttctctactaagaactctttcttaaatgtggtttgttactgtagcactttttacactgaaactatacttgaacagttccaactgtacatacatactgtatgaagcttgtcctctgactaggtttctaatttctatgtggaatttcctatc

SEQ ID NO: 164 - DNA, 514 bp, human
tgtggccttggaacgagtttcccatgagtaactgaggggaggggtactcagctccatctccaaagataaacctactccccacaagaactggcctttaatgtggtataactgttccgctgccttcttgtctgtgtgctaatataaatactgagtaccagcatgtccatttgaacatgcaaagggttaatcctgcttcctaaagcctcaagtacatgcctcctgcttagttcactttgtatcacatttcctaagctcccttttcccccagttttgggacactgtgcttacctccaaaaatctcatctcttccctggcattctccctaggctctgttttgcccagggctcccgctttttcttgctctagaggagcagtattcaaccttttagctatgatgacacataacaaaagatgtttatgtactaatagttgaaatctgcctttttctcattcaagaaggcatacaaatatctgagagtgactttgttgtatggctacccttgtgatctacagt

SEQ ID NO: 165 - DNA, 503 bp, human
tgaagctgggccagtcggtgaccaccattcccactgtgggtttcaacgtggagacggtgacttacaaaaatgtcaagttcaacgtatgggatgtgggcggccaggacaagatccggccgctctggcggcattactacactgggacccaaggtctcatcttcgtagtggactgcgccgaccgcgaccgcatcgatgaggctcgccaggagctgcaccgcattatcaatgaccgggagatgagggacgccataatcctcatcttcgccaacaagcaggacctgcccgatgccatgaaaccccacgagatccaggagaaactgggcctgacccggattcgggacaggaactggtatgtgcagccctcctgtgccacctcaggggacggactctatgaggggctcacatggttaacctctaactacaaatcttaatgagcattctccacccatcccctggaaggagagaaatcaaaaacccattcataggattatcgccaccatcac

SEQ ID NO: 166 - DNA, 160 bp, human
gaacctgtgtttggtaaagcaggaggcagagggctcgaaaagagtcttcatttttcctagtttttcataatgagcttctcttagaacaatcttctcccttctcaaatggactatgtcggctgattaattactgattgtatttgtaggatgcttttaagtg

SEQ ID NO: 167 - DNA, 344 bp, human
  misc_feature (95)..(95): n is a, c, g, or t
cccccatccacagttgtcagttgtgaagaggggccagatgacaccctaaagcaggctgatcgggcaatgggaggtggccaggcctgccagccccncggctctgccgcggtggnccgaggccgctgttgggggtttccctgtgtggaccatggggatcagagtctggcctgccaggctcctctccccgttttctcttttggtgggggggtgtgctgtgggtattcaccaccctgggacccccccccccccnngcccaccagacctgttctgacctcatgagtgtcaaacgtgccccccagttccttaggagatgtcatgacaacactacacgcacccaataaagc

SEQ ID NO: 168 - DNA, 467 bp, human
  misc_feature (31)..(32): n is a, c, g, or t
ggatcactaaataaccattgcaggtaccgtnngtgtaacattactcatttctgtatattccttttatgggaagatattttgccatggtaactaaaacttttcagttctacttttatgatgtgaatgaatgctacgttttattaaatattaccaggtcagtactatttttatactttattaagcaacaggggattttagtttaataggctcaaaataaaaagttcttaatggaacagttaaaaacaaaacactaacaatctttacgtgaaaatccccactaatagtgccacaataatttctatagaaatatctaaggtcattaaatagatttttgaagacggttcttcattgtgtcaggatgacctttcatatcattctcaccaacttgtagtgcccaccgttatttgtaactattaaaccatactaagtatgtttgtaaccagcattgtgatatattctgtacttgt

SEQ ID NO: 169 - DNA, 421 bp, human
ctcccgctatctccaaatcggacgttctttctagctgagatttttatttttccacatctgtagtgccatgaagcgattctgtctttgacttccaatggcaaacctgggtgatcgggaacaagcacgttgtacccttggctggacatggccaagacacaaggcattccacggcggcaagctgaccgcacagcagtctggcttgattttcagccgtcatcattgggttctgttttgacagctctgctgtcccatagggactgcgactgggaccaggtcaaccacgccaggggggtgtcaccagccttttctttttttctttctttttttttttttcctccttaagctgctgtcaatccaaaccattggcatcatcgtttcttttcaattaaaaccaacatatcagcaatagtctgctctcccc

SEQ ID NO: 170 - DNA, 531 bp, human
agagctctcaagtcacatcaaggtcagcgagcagaagatgaaacggtggttgggggaagggcacagcttcctctcttgtccccactacaggttttccctactaaacaaaggtgttggagagcagctttgggttctgtgctggttgttagaactcatctccaggtagcccacggatacgtggtttggcacagacacaagactcccagagttgtcctaacaataagtctgagcccatctcaacccacttttctccggtagtctttatgtatctgttagcacaatcacttcagttactgatgaattttgttgggatctgacttggggaaagggttatcagagcctagaggggcttaaaaagtaatcatttgatgtacataccacactccttggcttcctttctcttcccttaaccctttctgcttttcattaaccacattcctgcacaactcatttctgaaaacctaccatgtttctttacagagccatccaaaaattttttgtccctacatagcaattttctgtggcactgag

SEQ ID NO: 171 - DNA, 494 bp, human
  misc_feature (237)..(237): n is a, c, g, or t
gcccttgcccatgcgttgcaagaaaatgccacccagaaagaacagttgctcagggttcaacttgctacaagtattggcaaccctccagtttctttacttcaacagtgcaccaatattctttcacagggaagcaaaatacaaacaagagttggattattaatgttgctttgtacctggctaagcaattgtcccattgcagtaacgcattttcttcacaattcagccaatgttccattncttacaggacaaattgcagaaaatcttggagaagaagagcagttggtccaaggcttatgtgcccttttgttgggcatttcgatttatttcaatgataactcacttgagagctacatgaaagagaagctaaaacaactgattgagaagaggattggcaaagagaatttcatagagaaactaggatttattagcaaacatgagttgtattccagagcatctcagaaaccccagccaaactttcccagtccagaatacat

SEQ ID NO: 172 - DNA, 352 bp, human
atttccttggtttctgttaccactgttagagggagttgtatcaatttaacatatagtaaggatagtttaagtgtagggaggacaataattgtctgtaagctaaaatgggttatttataggactgatggaaatgatttcatctctgccatctctaaagcacttttcattgaacatgttagcctaggatacgtacagtaaattaacaatgatagcagcagatgcctagctcatcctgggtttgcttctgaccttgtcatgtgtgtgccaccaaacacgttattggcacctttttaaataagctctcatgatcaagatggtgatggtagagaagctgcccggaataaactgaatt

SEQ ID NO: 173 - DNA, 529 bp, human
gcgcttcttccaggacaagcgcccggtgcccacgtagcccctgccatggaaggaagaacgggacaaaggaggggcagcagggtgtgtgcatatgagacttgggggtccctccccaattttagtttcctgccaaaacgggagtgtgcagtcagggcctgcggtctggccccatgagtctccttccgtcctcagcgggcagggaacacctctggcttgtagaagggacggctcagtggctgcaccgacggtcctggaaatctcacatggtgggcactgcagcgttggaacgtgagcctcggatttcctggcccctctactgtaaatgtgccttagcctaagcctcccatcctgtgttagcgttgcctggtgcggggcagggcctaacaaggaaacctgggccctccaagccaggttgaggtctggtaacagaatgccaggaagggggcctggaagaccacctgccccggcccctctcctgcaggggccccacacaggcatgagggatggcccggccaaagtctaggcagaa

SEQ ID NO: 174 - DNA, 462 bp, human
ggtgctagtgaaactggaccttcggaagaaggcagcatgtattgctgagcagtatcatactgtgttgaaactccaaggccaggtttctgccaaaaagcgccttggtaccaaccaggaaaatcagcaaccaaaccaacaaccaccagggaagaaaccattccttcgaaatttacttccccgaacaccaacctgccaaagctcaacagactgcagcccttatgcccggatcctacgctcacggcgttcccctttactcaaatctgggccttttggcaaaaagtactaaggctgtggggaaagagaagagcagtcatggccctgaggtgggtcagctactctcctgaagaaataggtctcttttatgctttaccatatatcaggaattatatccaggatgcaatactcagacactagcttttttctcacttttgtattataaccacctatgtaatctcatgttgt

SEQ ID NO: 175 - DNA, 430 bp, human
  misc_feature (133)..(133): n is a, c, g, or t
gaggctacacagtagctcttcctgctactcggttaatgagcttggcaggttctttgtctcactgaattcttatcatggaaacagcagcagcagccgctaggaaatcttcaagtgtagtgtctgtgctaacccnagtggtaaatcccttagatcccctgctggtctctggcagtctccttgattttgggtaccatgtatattttccgctttgactttaacgctttctaggatagggtaagcacccttaattcaggcactgtccattagcttcctttgcaaaggctacttatggccggtcacaatccagcactcagacagagccaaggcaatatcctcttgcccatggctatgatgtcagacagtggatgggctccagcaacaagagacaaaataactaaaggcctttgctctcctctgacattgaggcctg

SEQ ID NO: 176 - DNA, 516 bp, human
tgagcaagtcaaccccaatctggaacaatgtccctcctcttagaatgtcccaactaaagaccagttaaaatattagggtacgttcttgtgaatttccactttccaggtagatgaccaaatttaggtggtcaagatataaaggtgtcagctagttttaagtgtgaaacttatttcactttcacactgccttcaggccagaagcaaaccaaatttaccaggtttggctggaggagttttgtgactcatcttttactggtttgaattttttcaaaccagtggctgatacctgccttgtacttagtaccttaataccaataacctaatggtacttaggcgagtaccatttgcacaatcactgttttacttatgagcagatacagatatatccaaacccttacctactaggtatcctgctagggttttcaattccaattcttgtattaagttttttcctttcagttttaggtgcgaaagtaatcagtcaatccaatatcccccatctttgtcttgaaacaa

SEQ ID NO: 177 - DNA, 420 bp, human
agcacccgttgagtccttttgagggtgatttgtcttacaactgactgacttagcaggaatttaattaggtcatatttggtgatgagacttatggagtgtgcctctctctcccaactgctgcttaaaatgcaaggacaagcaattagaagccatcctaaggtgcttacctcacacgccacccatgaggcttgtggccacagtggcacttgggtgtggctcctctgttatttgtcctcatgtgagaaagcagatcatctccaaatcttgccatttgtatacttttggtggagacttggatgtcatatcttctttgttttgggttttcttccctagcttattttgtggcttttaaagaagtggattgtattgtgagatcctgtgattcctggtggccagtatcctggattcctctaagatctt

SEQ ID NO: 178 - DNA, 512 bp, human
agtaatatcctttacagttctgtgaaaggacttattttttaactgtaatatttattagttttaaaatatttgtatctcatttgtaacaatttgttttaattttttatatatatgtttttatttttaaaaaacataccagttgaatggggttaaagctttcaatatcttaaaatatttataaaacatttcactgttgcaaaatcacttccaaaatgatagctatctaacaactaattactaatttttaaagaacaaatcacacatttaaaaaatctgtagaatttattttaactatgacctttaattgaaaataaataattaaaatatcagacatgttttggaaaagtcttaatttgagaacaccaaaggaaactaccccagaatctaatgtagttcgctattaataacaatgcattattgaaagtatattgcaaatacatgtttcctcatgaaatctaagtaattttgttgtggaatagtgtcactgttacatttcccccaagaagttcaat

SEQ ID NO: 179 - DNA, 502 bp, human
gaaaatgccttcaatttgtgttttaccagaattttgatactggtcagaaattttatactgccaacaaagaagactcaacttctcagatctataatgggatacattgtcatcctctagcaactcctatatagaaagttttaactgaatatgttacatataagaattaaattcttctcaaataattcttaacctcagtaatgagcctaaatttactctgcttggctctctacacatggcatttcagggtataagatgtagcatttcaatgtgtaagatatatgtactaaacatatgtgttgctatcttcatcattaacatccttcttttctattgcttggctgtaatttttgtaaagataaattatattgtttttttgtatgtgtgtttgtagtatatgttcagaaggcaagcatcttcattttgctagctttgcagaatcttaaaatgtgtactcgttatttctaatgatgtaaaaaaaaatccctagtcctgtttagcattt

SEQ ID NO: 180 - DNA, 231 bp, human
tgtagtgtatcttagtggcctgcctagctgcctctgggccaggctgacttctgatgtccacattagctcgtacctgaaccctgttgctgaatgccagccctgttctcctgtaactattatatacgccatggcctggggggcattgaaggaagtaagctctcagagatcctaacactggctgggaacctctgactcagagcatgtctttaaagagtccacatctggccaggc

SEQ ID NO: 181 - DNA, 559 bp, human
gacaaacggtcacatgtccatcgttatggcatgtgcagaaaaacagagttatccacacaagtcaggagcaaaacccaagtagatgcctctaggggcacatggcccctcacatcacaagccagaacctaagctaagcattttttaaattgagtttgagactagcctgggcaacatagtgagaccccatctctatttaaaaaatgaacaaaattacctgggcatagtggtgtatacctgtagtccctgctacttgggaggctcaggtgggaggatcacttgagccccaaagatggtggctgcagtaagccaagatcacaccactggcactccagcctgggcaacagagtgagaccctgtctcaaacaaacaacaacagtaacaacaaaaaaatatatagcgacttgaataggaaaccatagtatttcattgttttaatttgcatttattttatttctagtgaaacttttatatacgtatcggtcatttctatttccttttttgagaactgccaaatgttgccctagtagccaaaaatatcaggttgctattctgccttttc

SEQ ID NO: 182 - DNA, 482 bp, human
ccttgctactttactgatacctttatcattcctgagacagtttgctaatttaaaaatgtagcattccatttgtatttatttctctcccttgccaaaaagattttctaatactgcttgtaccagccagagaaagatccaaaacactactcagctctcttgcactgaggaaatttttccccctacattgactcctggcctacatcagccaaacttaaccttggtggggtttggatttgatagccaattagttctgtgctggttgcaaagaattgatatttagatggtttttaatactcagcagattgtcttcccatattgtgtcttttttatgttgcatgttgcttttgttatcagcctgattttttgctcagtatatgatagttctgctgatgttttgtttattgggcagacatatcttcattaagagtttttggaaaactcatcaaattcgatgaatacattttcttcataacccatttgga

SEQ ID NO: 183 - DNA, 551 bp, human
agcaaattgagcttgggtgatttttatggagacaataattagacaatactgtataattagttttacttaatagattatcatcttgtgagaagagatgtttaaacgtggtaaatcacttcatattacaaaacagttttacacttaatatgttaacattgggtgcaataatttagtagcattagctttagttacaaatataactggatctttctgctgacaacttaggttgtatgagttatgcttaaaagctttaaatctgatgtttcctgtacctgccacactatgttagaatgtgtccttcaaacatatcctcctgcaacttctcaaactgtactaaattgatatttcttgaagtctaactctgtgctaacagatctccattttaaatagaatacggttttaatttttgataagctgctgaattttaaagagagttttttggggccaccaaatattttggatcatgcagagaatatatattgtactgtagtaattttgtatttacatttgtatgatgtgacataatagatgtgaatgttaatcactgcttg

SEQ ID NO: 184 - DNA, 563 bp, human
gaaggcgcattatgttgtcgtgtgtttcagtttcacattaaactgaaccttttactaattgtgagctaaagagatatatatatatatgtgtgtgtatatatatatatctacatgtctttctgtagcctctgcatactactggctgtcatcacaccagcgtacagtagctaaatttttggtgcaattattagcaaatgataatgttcccttttgaacttttacattttggcatgacatttcagagtattgtgggaccatgagacaaaattaagtacgatcacattctttatttctcattttaaagaaatgatgttggtttaccttttcctagttgaagatagtaattaggtttctaagctgtatactgtgtttattggtggcagtgacacccaaagatagaggcaatggatagaaatttttaaactggaaagaaaacctgaattacactacattttcgaagtctcttgtaattatttgggatatcaacaaaatttgattcgtctgtctaatcccttgctagtattttaaatatgtctttaacacattgtatcctttaattcttc

SEQ ID NO: 185 - DNA, 500 bp, human
acaatgtcaaacatcacagccggccgccaggaccagatacagcaagttgtgaatcatggattagtcccattccttgtcagcgttctctctaaggcagattttaagacacaaaaggaagctgtgtgggccgtgaccaactataccagtggtggaacagttgaacagattgtgtaccttgttcactgtggcataatagaaccgttgatgaacctcttaactgcaaaagataccaagattattctggttatcctggatgccatttcaaatatctttcaggctgctgagaacctaggtgaaactgagaaacttagtataatgattgaagaatgtggaggcttagacaaaattgaagctctacaaaaccatgaaaatgagtctgtgtataaggcttcgttaagcttaattgagaagtatttctctgtagaggaagaggaagatcaaaacgttgtaccagaaactacctctgaaggctacactttccaagttcaggatggggctcc

SEQ ID NO: 186 - DNA, 293 bp, human
gaccatgcaagactgtcagtgccaacaaagacaacactaatcagcacatcgtacactggattgcagtgcttcccagattattgaaaaatgttacagacaacttgcctgatttttaaatgagcgtaaaaggccctctaacctatgcaggtttccccattatgcatatagaaaatgctagtatgttttgctcacttcatatgtaacaggtgcccttatgttgtgctgtatcctgtgctttttctgtgggaccattccattcaggagcaaagagcaccatgattccaatcttgtgt

SEQ ID NO: 187 - DNA, 404 bp, human
  misc_feature (227)..(227): n is a, c, g, or t
gtttagtgcgtttcttttccatactttctcccctaattcttttatttgaagaagagaacttaatggcaaataaacaacaaaccaggacatgcattttaataccctaaggaaaaatggaaccctcaaatataactcctcccacaaccaccctaatgtgcctcagtctggggcagcaggtggacttctcaatagttcttttccacattctctacaaatcacatctaccnttaaggaatatgttatgaatcctttctgttaattgagaaagcaatgttattgtctgtgatttccagtctttgctcattttattatccctgtcaaataatgtaatattggtacctgcagttgaatttgtaatattgtaattgaatttttagttgatcttcgatcagtttttatagcat

SEQ ID NO: 188 - DNA, 395 bp, human
  misc_feature (174)..(174): n is a, c, g, or t
ttccctccttgtcttatgttttggcttttgtgtatcagagcatctttctttgcagcatcctagtcttgcttcagtttctgtggtctcttttcatcctgcttgaagattcggagtgtggaaggaggctaggaagtctggtgcagtaggtgagcagacctcttgctgcccagcccnagggtgggtggggcgcacnaccnntgtctttgtgcatgcaaatctgatacacctggcgcatcctctggagagcacaacgcatggaaaggtctggaagctctgtgtagccattccttctgcagtcatcctacccaagtaaaagtaaccttggctatgttaccaccgttttggtcacccaggaggacatcttagcaagggtgcctgcgagggagtgtgggact

SEQ ID NO: 189 - DNA, 465 bp, human
aacacttccctatgttggagacccaatcagttcactcattcctggtcctggggagacgcccagccagtttcctccactgagaccacgctttgatccagttggcccacttccaggacctaaccccatcttgccagggcgaggcggccccaatgacagatttccctttagacccagcaggggtcggccaactgatggccggctgtcattcatgtgattgatttgtaatttcatttctggagctccatttgtttttgtttctaaactacagatgtcaactccttggggtgctgatctcgagtgttattttctgattgtggtgttgagagttgcactcccagaaaccttttaagagatacatttatagccctaggggtggtatgacccaaaggttcctctgtgacaaggttggccttgggaatagttggctgccaatctccctgctcttggttctcctctagattgaag

SEQ ID NO: 190 - DNA, 338 bp, human
acatgcttggtgtagcctatgaagccccttcgctggccactggttatggtgcatacttggctcagcctctgctgcgagaagttctggagaagcagccagtgctaagccagaccgaggcccgcgacttagtagaacgctgcatgcgagtgctgtactaccgagatgcccgttcttacaaccggtttcaaaccgccactgtcaccgaaaaaggtgttgaaatagagggaccattgtctacagagaccaactgggatattgcccacatgatcagtggctttgaatgaaatacagatgcattatccagaactgaagttgccctacttttaactttgaacttg

SEQ ID NO: 191 - DNA, 449 bp, human
aagatactcgtattaagcctatttataatctatatgcaatttcgtgccattcaggaattctgggtgggggccattacgtcacttatgccaaaaacccaaactgcaagtggtactgttacaatgacagcagctgtaaggaacttcacccggatgaaattgacaccgactctgcctacattcttttctatgagcagcaggggatagactatgcacaatttctgccaaagactgatggcaaaaagatggcagacacaagcagtatggatgaagactttgagtctgattacaaaaagtactgtgtgttacagtaaagctaccactctggctgctagacagcttggcggtgagggagatgactccttgtagctgacatttggcaaaagcgtcactgaaaggcaagctaaatgtagttattttatcctgtggccctgaagcacaaaataaaaatt

SEQ ID NO: 192 - DNA, 567 bp, human
gaaaccagctactcagggacttctcttgcctggagggtgcagtgatagctcctcctcactgccccactgtgctcctgggcctgtgaccccagtgctcaggcaccttgcactagaggcttctgactcctgggactttggagcttaccagagatgcagtccctcccaggaacctgttggagaggcaggacctgctgctttagagtgcggctgaacccgggccttgcgtccctgtttggccagagcaaggatctggcctggagaggcccatcctataccccttattagagccatgacagcctacagagtgaggtgaggtgctcccaccttcccagatggttcctttctgccccttcctggaaggaaaggtgaggctgccaatagcctcctggcaccagccagacctcacccttgaccaacctctcggggctgggggttcattcctggggcactgtggcctggttttgctttgaaaccaagaaagagcaaagggaacccagcagttctgagtgagttctgagccagccctacctcaggctggctgttgagacatgctacaattttcatttt

SEQ ID NO: 193 - DNA, 533 bp, human
tgcttttccctttgtgagtcaatacatagtgctgctgtgtgctttttttgtgtgtatttgctaatttttattaattttagtttttcattaaataaatttgacttttctgtaattcaggtttttcctttttttgtaccattttaaagttagtatcttttgatatgcatatttgtttatggtaaaaaatttataacgtgttcaatattttcttttcccccattaatcagttcattagaaatattttaaaatcagctattttgtgaagccatgagttccagaaagtaaaggtgacatcggaaaaataatcccaagctatttaaagcatctataaggtgctctctttctgtcttctacagatgagtcacacctttgagcttaatcttgaaggttagagataaattgatttttataaatactgcaaatcaggcttttgtttcctttttcagatatcttggacaaatcacatattttaaaatttgttcttgtatttattggttttgcagaagaaggcatcgtcatgcacagtatttgta

SEQ ID NO: 194 - DNA, 530 bp, human
agactgtcagtactgggagcgcctgctggccaaggaatgcaggctgtactatctgcgaaatggagaacgtatttcagtgtcggcagcctccaagctgctgtccaacatgatgtgccagtaccggggcatgggcctctctatgggcagtatgatctgtggctgggataagaagggtcctggactctactacgtggatgaacatgggactcggctctcaggaaatatgttctccacgggtagtgggaacacttatgcctacggggtcatggacagtggctatcggcctaatcttagccctgaagaggcctatgaccttggccgcagggctattgcttatgccactcacagagacagctattctggaggcgttgtcaatatgtaccacatgaaggaagatggttgggtgaaagtagaaagtacagatgtcagtgacctgctgcaccagtaccgggaagccaatcaataatggtggtggtggcagctgggcaggtctcctctgggaggtcttggccgactcagggacctaagcc

SEQ ID NO: 195 - DNA, 498 bp, human
ttgccgatatttctcaggtctacacacagaatgctgaaatgaggcctcttggttgttgtatgattttaattggtatagatgaagagcaaggccctcaggtatataagtgtgatcctgcaggttactactgtgggtttaaagccactgcagcgggagttaaacaaactgagtcaaccagcttccttgaaaaaaaagtgaagaagaaatttgattggacatttgaacagacagtggaaactgcaattacatgcctgtctactgttctatcaattgatttcaaaccttcagaaatagaagttggagtagtgacagttgaaaatcctaaattcaggattcttacagaagcagagattgatgctcaccttgttgctctagcagagagagactaaacattgtcgttagtttaccagatccgtgatgccacttacctgtgtgtttggtaacaacaaaccaacatcatggaggtccctggattgaaaaaggagcctctcccactcc

SEQ ID NO: 196 - DNA, 313 bp, human
gtatttgaagcaagttctcggccagatggtgattgatgaggagcttctgggagatggacacagctatagtcctagagctattcattcatggctgaccagggccatgtacagccggcgctcgaagatgaaccctttgtggaacaccatggtcatcggaggctatgctgatggagagagcttcctcggttatgtggacatgcttggtgtagcctatgaagccccttcgctggccactggttatggtgcatacttggctcagcctctgctgcgagaagttctggagaagcagccagtgctaagccagaccgaggcc

SEQ ID NO: 197 - DNA, 563 bp, human
  misc_feature (84)..(84): n is a, c, g, or t
gactccagccgctgttggctgggaatcgaagttagaggtgacttccaaaggccccccgagccggcagtgccccccaccaccccnccagcgactctgcggtgccagtgccttgttggcttttccggctacgcaccctgcagtcactgagctctcggtctgacgtctgatgtttgtggtttgtttataacacggggccttacctggggaattcagctggtttgaatatttgtagcccgctcccagaatgtcttattttgtaatgactgaactacatttagtaatagttacacatgtatatggttaatacatatggaaattcaatatattttgtagttaacgtattctgaagtaacggatgtttctcgccaatcgtagtgacttcagctaacgaaatgttcttttgtagtaccacggtcctcggcctaacgaaggacgtgaaccttgtaagaggagagctctgaaacgcggtcacctttgtttagtggaagggaaagtgtgttcccggcatgaggtgcctcggaattagtaaagaattgtgggcaatggattaaccactgtatcta

SEQ ID NO: 198 - DNA, 534 bp, human
aaactagctcaacaatactatcttgtgtaccaagaacccattcctacagctcagctggtacagagagtagcttctgtgatgcaagaatatactcagtcaggtggtgttcgtccatttggagtttctttacttatttgtggttggaatgagggacgaccatatttatttcagtcagatccatctggagcttactttgcctggaaagctacagcaatgggaaagaactatgtgaatgggaagactttccttgagaaaagatataatgaagatctggaacttgaagatgccattcatacagccatcttaaccctaaaggaaagctttgaagggcaaatgacagaggataacatagaagttggaatctgcaatgaagctggatttaggaggcttactccaactgaagttaaggattacttggctgccatagcataacaatgaagtgactgaaaaatccagaatttcagataatctatctacttaaacatgtttaaagtatgttttgttttgcagactttttgcatacttatttctaca

SEQ ID NO: 199 - DNA, 512 bp, human
tagcactttggttccacatctgtctgggtaaaccatgaagaaaatgaagctgctgcctcaatcgacccagacagcagccataggcagataaagatttggtttcaccctggtggtggtaggcatcgtgtgtgactttttttcctctaatatcaattttacagtacggaaatagtattttaaaatagtattggctaataaattatgaattctataaagtagtaagacttggtatggttggagtgtaggaatgaatattcatgaaatgtttcttattgcttttccttccctaattcatacaatgaatgtatttggaatacttacatattataaaataaactatacctcttcaagaggtatcctgttctgtaagatcagatgtttttattgcaggtcaatataatactgccagagacagaaaatacccccttatcagtcccttagtgcctctttctgtttgtggcatggtgagaaaacccatgctgaaaagattgtactttgtgatcccaatcaga

SEQ ID NO: 200 - DNA, 981 bp, human
  misc_feature (76)..(78): n is a, c, g, or t
tgctcggccgctactttgaggaagaggaagggcaggagccgggaggcatggaggacgcacagggccccgagccagnnnagncnngnnnnnnnnnnctccaggattgggaggaccaggtccgctgcgacatccgccagttcctgtccctgaggccagaggagaagttctccagcagggctgtggcccgcatcttccacggcatcggaagcccctgctacccggcccaggtgtacgggcaggaccgacgcttctggagaaaatacctgcacctgagcttccatgccctggtgggcctggccacggaagagctcctgcaggtggcccgctgactgcactgcattgggggatgtcgggtagagctggggttgtcagaggctagggcagtgactgaggacctgggcaaaacctgccacagggtgtgggaacgaggaggctgctgtagtgttgaatacttggccccatgagccatgcctttctgtatagtacacatgatatttcggaattggttttactgttcttcagcaactattgtacaaaatgttcacatttaatttttctttcttcttttaagaacatattataaaaagaatactttcttggttgggcttttaatcctgtgtgtgattactagtaggaacatgagatgtgacattctaaatcttgggagaaaaaataatattaggaaaaaaatatttatgcaggaagagtagcactcactgaatagttttaaatgactgagtggtatgcttacaattgtcatgtctagatttaaattttaagtctgagattttaaatgtttttgagcttagaaaacccagttagatgcaatttggtcattaataccatgacatcttgcttataaatattccattgctctgtagttcaaatctgttagctttgtgaaaattcatcactgtgatgtttgtattctttttttttttctgtttaacagaatatgagctgtctgtcatttacctactt

SEQ ID NO: 201 - DNA, 477 bp, human
cttcccattctgcaaccatggcaagcgttccaccatgaaaacagtattgaagattggacctaacaatggaaagaatttttttgtgtgtcctcttgggaaggaaaaacaatgcaattttttccagtgggcagaaaatgggccaggaataaaaattattcctggatgctaatatctgtagattctctggcatttagtctcttcaaactgtgtataatgtttggtcctcctctgtttcatagaaaagtcatagaatatctatgatacattgaaaagttactgcaatatttgagaactgttctttttttttcttgtgtgtgccatctttccattgttggctacgtcttttcttttgccttgatgaacgttctatgtatttcatcggatatacagcatattccatttaggatgtgtatttaatgcatttagtaatgacgataaagtgtttttagtatgcttttagtctcttgtaactgggga

SEQ ID NO: 202 - DNA, 438 bp, human
gttttatggcatatgtctccgatttagatggttattctctagaaaatagtatttaaagacattttatgaaatcttcattgtcaaaacctttaataaaagtggaaatattttgaaatgccctttttcttgataccactcatccacgtgttcctgattgtccacatttcatgataaaatgagagctctgcagagaatgttagcctttctgttgtaaatgtaatcttcaagtagtcactttttgttaagttctttagaaagtagttgtcaagtacttagtcatccctattatgatatgagatagtacagcttttcaggaagcttagatctgaatttactttgaaaaacaattgtaatgaatattttatatttacattgagaatttcaactagcttctgatcaatttttaataaaaaattttcaaatcatgttagctgttaa

SEQ ID NO: 203 - DNA, 512 bp, human
agcaggatgtagagcgccacttctctctgggcgagttgaaggagctgtttatcctggatgaagctagcctcagtgacacacatgacaggttgcactgccgacgttgtgtcaacagccgtcagatccggccaccccctgatggttctgactgcacttcagacctggcagggtggaaccactgcactgataagtgggggctccgggatgaggtactccaggctgcctgggatgctgcctccactgctatcaccttcgtcttccaccagcattctcatgaggaacagcggggcctccgctgataaccagctggtctgggtgtagctcttagaggaaggagatagggaaaaggggctccttgctccacagggccctgttgaattttgttctctgggagaaaatcatcaagaagggctgcatgatgtttgcccaaaatttattttataagaaaaacttttttggttaaaaaaaagaataaaggtatgaaagggctggtgacagtcagggatgccccc

SEQ ID NO: 204 - DNA, 361 bp, human
ttcaagaaccggtttccaaagacagtcttctaattcctcattagtaataagtaaaatgtttattgttgtagctctggtatataatccattcctcttaaaatataagacctctggcatgaatatttcatatctataaaatgacagatcccaccaggaaggaagctgttgctttctttgaggtgatttttttcctttgctccctgttgctgaaaccatacagcttcataaataattttgcttgctgaaggaagaaaaagtgtttttcataaacccattatccaggactgtttatagctgttggaaggactaggtcttccctagcccccccagtgtgcaagggcagtgaagacttgattgtaca

SEQ ID NO: 205 - DNA, 531 bp, human
gtgaatgtccatattgtagtaagccaattaccttaaaaatgtctggaaggaaacactgaaataagaatacaacatttcggtgaagagctggaaacttaaaaaattatcaaaaggaattttggtatcatcttcagagaaaaaataaagcaagaaatactaacatcaaaaggacaggtatgatgatgcgataataataaacatctgcgtttgtctcttcactaagagtaaactgggaaattgtaggccaaagtccagttgaactttctaagtctgtgatccccgtgctgactgtggaagtgtatttataccaagatggagatcttgacttcttgaatatatctggactggtaaaatcttgatgaggctcataaaatgagtttgggaattgtgtatagctgattttttgtgggaaactgtttacttcattcaaaggttcttgagactcttgatatttctgtcttctccttgtgctttcctatggaaaaaatacatatatagtttagtttgttagacgtgagttatccaagtatt

SEQ ID NO: 206 - DNA, 486 bp, human
ggaaattcctcaactttctcaacgagtcatgtaacgttacactggcctccataaagcaccgattaagaaagctaaagaataagatgttgtatttcttttaaaataatttaaaatatattaaattttcctaaggcaggttttgtttgaatgaggtgtcttgattagataactacatgccactgaaggagaacagtacttttaaggagctatttttgtttcgcagtagagttgaaaccagctgattaatccaatgaagttaaacagcagagacagatctcgtacataagagtatcagctaaagtcatgactacaccaattctttacacaattaagacatctttgcaactgttcaatttaagtactatggtttttagataatcgagaaaaacacagttgtaccttaacatctgttgagaaaatacaaataaatatgatgctaataaatggccactgataactcagtagccatctgaatagtcatgcggt

SEQ ID NO: 207 - DNA, 295 bp, human
agatttgtcagccctatctcaaacttgaatcaaaatttctggggtgtgggcacaataatctgtaattttctttgtttatacttcccctgatgccactggttccgatgccactggctggggggcctgctttgaaatgcttgtctgcagagtcacagcagccatgaaaaccttatgaccgtgcaaatgagctctgctctaaaattgttgacattcatgtctctgagttacaaaagtgctaattcactacatgtaattgtgtaagtaaacattgtgcctttactacttctttatgtaa

SEQ ID NO: 208 - DNA, 568 bp, human
gcattgagtagggccggccagtgcaaggccactgcctgccgaggacgtgcccgggaccgtgcagtctgcgctttcctctcccgcctgcctagcccaggatgaagggctgggtggccacaactgggatgccacctggagccccacccaggagctggccgcggcaccttacgcttcagctgttgatccgctggtcccctcttttggggtagatgcggccccgatcaggcctgactcgctgctctttttgttcccttctgtctgctcgaaccacttgcctcgggctaatccctccctcttcctccacccggcactggggaagtcaagaatggggcctggggctctcagggagaactgcttcccctggcagagctgggtggcagctcttcctcccaccggacaccgacccgcccgctgctgtgccctgggagtgctgccctcttaccatgcacacgggtgctctccttttgggctgcatgctattccattttgcagccagaccgatgtgtatttaaccagtcactattgatggacatttgggttgtttcccatctttttgttaccataaata

SEQ ID NO: 209 - DNA, 354 bp, human
tgtctgaggtggcatcacttctcttcagccagacagcactggccagtttggagtctgtccatcctgcaggccacaagctctggatgaggaacttgaggcaagtcaccagcccctgatcatttcgcctaaaagagcaaggactagagttcctgacctccaggccagtccctgatccctgacctaatgttatcgcggaatgatgaggctgccagtgcccagcagtgagggacctgacctcaggggcagagacaagagaggcaccaaggaggatccaagagctgactatgcctgcattgctgagaacaaacccacctgagcaccccagacaccttcctcaacccaggcgggtggaca

SEQ ID NO: 210 - DNA, 480 bp, human
gcactggccagtttggagtctgtccatcctgcaggccacaagctctggatgaggaacttgaggcaagtcaccagcccctgatcatttcgcctaaaagagcaaggactagagttcctgacctccaggccagtccctgatccctgacctaatgttatcgcggaatgatgatatatgtatctacgggggcctggggctgggcgggctcctgcttctggcagtggtccttctgtccgcctgcctgtgttggctgcatcgaagagtaaagaggctggagaggagctggacccagggctcctcagagcaggaactccactatgcatctctgcagaggctgccagtgcccagcagtgagggacctgacctcaggggcagagacaagagaggcaccaaggaggatccaagagctgactatgcctgcattgctgagaacaaacccacctgagcaccccagacaccttcctcaacccaggcgggtggaca

SEQ ID NO: 211 - DNA, 501 bp, human
agactggggtggtgtccacaggcctgatccacaatggagactggaccttccagaccctggtgatgctggaaacagttcctcggagtggagaggtttacacctgccaagtggagcacccaagcgtgacaagccctctcacagtggaatggagagcacggtctgaatctgcacagagcaagatgctgagtggagtcgggggctttgtgctgggcctgctcttccttggggccgggctgttcatctacttcaggaatcagaaaggacactctggacttcagccaagaggattcctgagctgaagtgcagatgacacattcaaagaagaactttctgccccagctttgcaggatgaaaagctttccctcctggctgttattcttccacaagagagggctttctcaggacctggttgctactggttcagcaactgcagaaaatgtcctcccttgtggcttcctcagctcctgttcttggcctgaagccccacagctttgatggcag

SEQ ID NO: 212 - DNA, 444 bp, human
agcccacctgctggatgaggaacttgaggcaagtcaccagcccctgatcatttcgcctaaaagagcaaggactagagttcctgacctccaggccagtccctgatccctgacctaatgttatcgcggaatgatgatatatgtatctacgggggcctggggctgggcgggctcctgcttctggcagtggtccttctgtccgcctgcctgtgttggctgcatcgaagagcaccttctgtcctggtcccaggcccagggctcctcagagcaggaactccactatgcatctctgcagaggctgccagtgcccagcagtgagggacctgacctcaggggcagagacaagagaggcaccaaggaggatccaagagctgactatgcctgcattgctgagaacaaacccacctgagcaccccagacaccttcctcaacccaggcgggtggaca

SEQ ID NO: 213 - DNA, 445 bp, human
gaaggagacggtctggcggcttgaagaatttggacgatttgccagctttgaggctcaaggtgcattggccaacatagctgtggacaaagccaacttggaaatcatgacaaagcgctccaactatactccgatcaccaatgacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctccaagccctctcccagagactacagagaacgtggtgtgtgccctgggcctgactgtgggtctggtgggcatcattattgggaccatc

SEQ ID NO: 214 - DNA, 398 bp, human
ctgtgtatccttcaaagacccagcccctgcagcaccataacctcctggtctgttctgtgagtggtttctatccaggcagcattgaagtcaggtggttccggaatggccaggaagagaagactggggtggtgtccacaggcctgatccacaatggagactggaccttccagaccctggtgatgctggaaacagttcctcggagtggagaggtttacacctgccaagtggagcacccaagcgtgacaagccctctcacagtggaatggagagcacggtctgaatctgcacagagcaagatgctgagtggagtcgggggctttgtgctgggcctgctcttccttggggccgggctgttcatctacttcaggaatcagaaaggacactctggacttcagcca

SEQ ID NO: 215 - DNA, 446 bp, human
aaatacccagggttctcagaatggaataaatatcccttttaatagttatatatacagatatacaactgttagctttaattggcagctctcttcttttttcttcttttcactggctttttacttggtgctttttcttgttttgcactggtggtctgtgttctgtgaataaagcaaagtaagaatttactaagagtatgttaagttttggattattgaaataagaggcatttcttagttttccagtaggatctaaaatgtgtcagctatgagtaagactggcatccaagaagtttatattatagatttaggtcctaatttttataaatcacaaggtaaaaaaatcacagaacagatggatctctaatgaaaaagggatgtctttttgtttatagtcatgtggcaagatgagagtaaaaccagagagcaaacctctataagtgttgagt

SEQ ID NO: 216 - DNA, 101 bp, human
cgatcaccaatgtacctccagaggtaactgtgctcacgaacagccctgtggaactgagagagcccaacgtcctcatctgtttcatagacaagttcacccca

SEQ ID NO: 217 - DNA, 131 bp, human
  misc_feature (85)..(85): n is a, c, g, or t
gcactgggaggcccaagagccaatccagatgcctgagacaacggagactgtgctctgtgccctgggcctggtgctgggcctagtnggcatcatcgtgggcaccgtcctcatcataaagtctctgcgttctg

SEQ ID NO: 218 - DNA, 448 bp, human
  misc_feature (28)..(28): n is a, c, g, or t
gccgagtactggaacagccagaaggacntcctggaagacnagcgggccnnggtggacacctactgcagacacaactacggggttgnngagagcttcacngtgcagcggcgagtccatcctnaggtgactgtgtatcctncaaagacccagcccctgcagcaccacaanctcctggtctgntctgtgagtggtttctatccaggcagcattgnaagtcaggtggttncggaanggcnaggaagagaagnctggggntggtgtccacnggcctgatccagaatggagactggaccttccagaccctggtgatgctngaaacagttcctcggagtggagaggtttacacctgccaagtggagcacccaagcntnacgagccctctcacagtggaatggagngcacggtctgaatctgcacagagcaagatgctgagtggagtcgggggctt

SEQ ID NO: 219 - DNA, 424 bp, human
ctgttttgtcagtaatctcttcccacccatgctgacagtgaactggcagcatcattccgtccctgtggaaggatttgggcctacttttgtctcagctgtcgatggactcagcttccaggccttttcttacttaaacttcacaccagaaccttctgacattttctcctgcattgtgactcacgaaattgaccgctacacagcaattgcctattgggtaccccggaacgcactgccctcagatctgctggagaatgtgctgtgtggcgtggcctttggcctgggtgtgctgggcatcatcgtgggcattgttctcatcatctacttccggaagccttgctcaggtgactgattcttccagaccagagtttgatgccagcagcttcggccatccaaacagaggatgctcagatttctcacatcctgc

SEQ ID NO: 220 - DNA, 294 bp, human
cccaactctgagtggcaccaatgatgctgaagactgctgcctgtctgtgacccagaaacccatccctgggtacatcgtgaggaacttccactaccttctcatcaaggatggctgcagggtgcctgctgtagtgttcaccacactgaggggccgccagctctgtgcacccccagaccagccctgggtagaacgcatcatccagagactgcagaggacctcagccaagatgaagcgccgcagcagttaacctatgaccgtgcagagggagccccgagtccgagtcaagcattgtga

SEQ ID NO: 221 - DNA, 544 bp, human
  misc_feature (174)..(174): n is a, c, g, or t
tgtgccttcattcatgggttaatggattaatgggttatcacaggaatgggactggtggctttataagaagaggaaaagagaactgagctagcatgcccagcccacagagagcctccactagagtgatgctaagtggaaatgtgaggtgcagctgccacagagggcccccaccangggaaatgtctagtgtctagtggatccaggccacaggagagagtgccttgtggagcgctgggagcaggacctgaccaccaccaggaccccagaactgtggagtcagtggcagcatgcagcgcccccttgggaaagctttaggcaccagcctgcaacccattcgagcagccacgtaggctgcacccagcaaagccacaggcacggggctacctgangccttgggggcccaatccctgctccagtgtgtccgtgaggcagcacacgaagtcaaaagagattattctcttcccacagataccttttctctcccatgaccctttaacagcatctgcttcattcccctcaccttcccaggctgatctgaggtaaa

SEQ ID NO: 222 - DNA, 364 bp, human
cagcacctagaaccatccagggctgaaaagtcccctccaaaccacgtgatgaggaacttgaggcaagtcaccagcccctgatcatttcgcctaaaagagcaaggactagagttcctgacctccaggccagtccctgatccctgacctaatgttatcgcggaatgatgacccagggctcctcagagcaggaactccactatgcatctctgcagaggctgccagtgcccagcagtgagggacctgacctcaggggcagagacaagagaggcaccaaggaggatccaagagctgactatgcctgcattgctgagaacaaacccacctgagcaccccagacaccttcctcaacccaggcgggtggaca

SEQ ID NO: 223 - DNA, 529 bp, human
gcctgacgccgagtactggaacagccagaaggaagtcctggagaggacccgggcggagttggacacggtgtgcagacacaactaccagttggagctccgcacgaccttgcagcggcgagtggagcccacagtgaccatctccccatccaggacagaggccctcaaccaccacaacctgctggtctgctcagtgacagatttctatccagcccagatcaaagtccggtggtttcggaatgaccaggaggagacaaccggcgttgtgtccaccccccttattaggaacggtgactggaccttccagatcctggtgatgctggaaatgactccccagcatggagacgtctacacctgccacgtggagcaccccagcctccagaaccccatcaccgtggagtggcgggctcagtctgaatctgcccagagcaagatgctgagtggcattggaggcttcgtgctggggctcatcttcctcgggctgggccttattatccatcacaggagtcagaaagggctcctgcactgactc

SEQ ID NO: 224 - DNA, 505 bp, human
  misc_feature (281)..(281): n is a, c, g, or t
agaacagcagctttctagggacagctggaaagggacttaatgtgtttgactatttcttacgagggttctacttatttatgtatttatttttgaaagcttgtattttaatattttacatgctgttatttaaagatgtgagtgtgtttcatcaaacatagctcagtcctgattatttaattggaatatgatgggttttaaatgtgtcattaaactaatatttagtgggagaccataatgtgtcagccaccttgataaatgacagggtggggaactggagggtngggggattgaaatgcaagcaattagtggatcactgttagggtaagggaatgtatgtacacatctattttttatacttttttttttaaaaaagaatgtcagttgttatttattcaaattatctcacattatgtgttcaacatttttatgctgaagtttcccttagacattttatgtcttgcttgtagggcataatgccttgtttaatgtccattctgcagcgttt

SEQ ID NO: 225 - DNA, 468 bp, human
tgtccacaggcctgatccagaatggagactggaccttccagaccctggtgatgctggaaacagttcctcggagtggagaggtttacacctgccaagtggagcacccaagcctgacgagccctctcacagtggaatggagagcacggtctgaatctgcacagagcaagatgctgagtggagtcgggggcttcgtgctgggcctgctcttccttggggccgggctgttcatctacttcaggaatcagaaaggacactctggacttcagccaacaggtaataccttttcatcctctttaagaaacagatttggaggccaggcgcagtggctcacgcctgtaatcccagcactttgggaggccgaggcgggcgaatcatgaggtcaggagttcgagaccagcctgaccaacgtggtgaaaccccgtctctactaaaaatacaaaaaaaaatcagtcgggcgtggtggtgtgc

SEQ ID NO: 226 - DNA, 479 bp, human
ctgcttggccttgggttcattcaagctcacgatgctgttcccacgtttcccgggatatatattctctcccctccgttgggccccagccttctttgcttgcctctctgtttgtaaccttgtcgacaaagaggtagaaaagattgggtctaggatatggtgggtggacaggggccccgggacttggagggttggtcctcttgcctcctggaaaaaacaaaaacaaaaaactgcagtgaaagacaagctgcaaatcagccatgtgctgcgtgcctgtggaatctggagtgaggggtaaaagctgatctggtttgactccgctggaggtggggcctggagcaggccttgcgctgttgcgtaactggctgtgttctggtgaggccttgctcccaaccccacacgctcctccctctgaggcgtgaggactcgcagtcaggggcagctgaccatggaagattgagagcccaaggtttaaacttctt

SEQ ID NO: 227 - DNA, 550 bp, human
aaagcctgagacagctgccttgtgtgcgactgagatgcacagctgccttgtgtgcgactgagatgcaggatttcctcacgcctcccctatgtgtcttaggggactctggcttctctttttgcaagggcctctgaatctgtctgtgtccctgttagcacaatgtgaggaggtagagaaacagtccacctctgtgtctaccatgacccccttcctcacactgacctgtgttccttccctgttctcttttctattaaaaataagaacctgggcagagtgcggcagctcatgcctgtaatcccagcacttagggaggccgaggagggcagatcacgaggtcaggagatcgaaaccatcctggctaacacggtgaaaccccgtctctactaaaaaatacaaaaaattagctgggcgcagaggcacgggcctgtagtcccagctactcaggaggcggaggcatggagaatggcgtcaacccgggaggcggaggttgcagtgagccaggattgtgcgactgcactccagcctgggtgacagggtgaaacgccatctc

SEQ ID NO: 228 - DNA, 542 bp, human
cgaggcacgtaccacaacaagtccttcttcaccgacgatgacaagcaagaccacctcagctgggagtggaacctgtcgattaagaaggagtggacagaatgaatgcatccaccccgttccccacccttgccacctggaagaattctctcaggcgtgttcagcaccctgtccctcctccctgtccacagctgggtccctcttcaacactgccacatttccttattgatcgatcttttcccaccctgtcactcaacgtggtccctagaacaagaggcttaaaaccgggctttcacccaacctgctccctctgatcctccatcagggccagatcttccacgtctccatctcagtacacaatcatttaatatttccctgtcttacccctattcaagcaactagaggccagaaaatgggcaaattatcactaacaggtctttgactcaggttccagtagttcattctaatgcctagattcttttgtggttgttgctggcccaatgagtccctagtcacatcccctgccagagggagttcttcttttg

SEQ ID NO: 229 - DNA, 330 bp, human
  misc_feature (55)..(55): n is a, c, g, or t
gacatcagttctgcttggcacaagtatcgtggatcaaaaacagagtattgaagangtggaagacacgcctaaaccaactctggtgacatttgctttaccttatacataaaatccttgntctgcatcttcttaaacaccgtccatgtcccataagggaagcatgcttttatttaaacagtttatactagcaaagatactgacccctttaggnaatactttttccccatcttccagagattttttttttcctgctttggctacatatccatcattgtttatttttgaaactataatccagatacttctttttcatggattcccgagatcacc

SEQ ID NO: 230 - DNA, 505 bp, human
agcccagtttcactgccatatactcttcaaggactttctgaagcctcacttatgagatgcctgaagccaggccatggctataaacaattacatggctctaaaaagttttgccctttttaaggaaggcactaaaaagagctgtcctggtatctagacccatcttctttttgaaatcagcatactcaatgttactatctgcttttggttataatgtgtttttaattatctaaagtatgaagcattttctggggttatgatggccttacctttattaggaagtatggttttattttgatagtagcttcctcctctggtggtgttaatcatttcatttttacccttactgtttgagtttctctcacattactgtatatactttgcctttccataatcactcagtgattgcaatttgcacaagtttttttaaattatgggaatcaagatttaatcctagagatttggtgtacaattcaggctttggatgtttctttagcagttttgtgat

SEQ ID NO: 231 - DNA, 429 bp, human
cagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagag

SEQ ID NO: 232 - DNA, 472 bp, human
cgacagtgcccaggggtctgagtctcacagcttgtaaagcctgagacagctgccttgtgtgcgactgagatgcacagctgccttgtgtgcgactgagatgcaggatttcctcacgcctcccctatgtgtcttaggggactctggcttctctttttgcaagggcctctgaatctgtctgtgtccctgttagcacaatgtgaggaggtcagagaaacagtccacctctgtgtctaccatgacccccttcctcacatgacctgtgttccttccctgttctcttttctattaaaaataagaacctgggcagagtgcggcagtcatgcctgtaatcccagcattagggaggccgaggagggcagatcagaggtcaggagatcgaaaccatcctgctaagaggtgaaccgtctctactaaaaatacaaaaattagctggcgcagaggcaggctgtgatccagctactcaggagcgagc

SEQ ID NO: 233 - DNA, 489 bp, human
ggcagcattcaagtccgatggttcctgaatggacaggaggaaacagctggggtcgtgtccaccaacctgatccgtaatggagactggaccatccagatcctggtgatgctggaaatgaccccccagcagggagacgtctacatctgccaagtggagcacaccagcctggatagttccgtcaccgtggagtggaaggcacagtctgattctgcccagagtaagacattgacgggagctgggggcttcgtgctggggctcatcatctgtggagtgggactcttcatgcacaggaggagcaagaaagttcaacgaggatctgcataaacagggttcctgacctcaccgaaaagactaatgtgccttagaacaagcatttgctgtgttttgttaacacctggttccaggacagaccctcagcttcccaagaggatactgctgccaagaagttgctctgaagtcagtttctatcgttctgctctttgattcaaa

SEQ ID NO: 234 - DNA, 482 bp, human
caccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggaagcagtcttccctgcccaccatccccatcatgggtatcgttgctggcctggttgtccttgcagctgtagtcactggagctgcggtcgctgctgtgctgtggagaaagaagagctcagattgaaaaggagggagctactctcaggctgcaagcagtgacagtgcccagggctctaatgtgtctctcacggcttgtaaattgtgaaacagctgccctgtgtgggactgagt

SEQ ID NO: 235 - DNA, 414 bp, human
  misc_feature (157)..(157): n is a, c, g, or t
gtatcaaaatcttccaattatcatgctcacctgaaagaggtatgctctcttaggaatacagtttctagcattaaacaaataaacaaggggagaaaataaaactcaaggagtgaaaatcaggaggtgtaataaaatgttcctcgcattcccccccgcntttttttttttttttgactttgccttggagagccagagcttccgcattttctttactattctttttaaaaaaagtttcactgtgtagagaacatatatgcataaacataggtcaattatatgtctccattagaaaaataataattggaaaacatgttctagaactagttacaaaaataatttaaggtgaaatctctaatatttataaaagtagcaaaataaatgcataattaaaatatatttggacataacagactt

SEQ ID NO: 236 - DNA, 433 bp, human
tatgattaactctacctgcacactgtcctattatattcattctttttgaaatgtcaaccccaagttagttcaatctggattcatatttaatttgaaggtagaatgttttcaaatgttctccagtcattatgttaatatttctgaggagcctgcaacatgccagccactgtgatagaggctggcggatccaagcaaatggccaatgagatcattgtgaaggcaggggaatgtatgtgcacatctgttttgtaactgtttagatgaatgtcagttgttatttattgaaatgatttcacagtgtgtggtcaacatttctcatgttgaaactttaagaactaaaatgttctaaatatcccttggacattttatgtctttcttgtaaggcatactgccttgtttaatggtagttttacagtgtttctggcttagaaca

SEQ ID NO: 237 - DNA, 475 bp, human
gagagctcgctttgagtgactgggttttgtgattgcctctgaagcctatgtatgccatggaggcactaacaaactctgaggtttccgaaatcagaagcgaaaaaatcagtgaataaaccatcatcttgccactaccccctcctgaagccacagcagggtttcaggttccaatcagaactgttggcaaggtgacatttccatgcataaatgcgatccacagaaggtcctggtggtatttgtaactttttgcaaggcatttttttatatatatttttgtgcacatttttttttacgtttctttagaaaacaaatgtatttcaaaatatatttatagtcgaacaattcatatatttgaagtggagccatatgaatgtcagtagtttatacttctctattatctcaaactactggcaatttgtaaagaaatatatatgatatataaatgtgattgcagcttttcaatgttagccacagt

SEQ ID NO: 238 - DNA, 536 bp, human
ttgcagccttcctacttgggctaatcttccttctggtgggaatcgtcatccagctaagggctcagaaaggatatgtgaggacgcagatgtctggtaatgaggtctcaagagctgttctgctccctcagtcatgctaaggtcctcactaagcttgctctctctggagcctgaagtagtgatgagtagtctgggccctgggtgaggtaaaggacattcatgaggtcaatgttctgggaataactctcttccctgatccttggaggagcccgaactgattctggagctctgtgttctgagatcatgcatctcccacccatctgcccttctcccttctacgtgtacatcattaatccccattgccaagggcattgtccagaaactcccctgagaccttactccttccagccccaaatcatttacttttctgtggtccagccctactcctataagtcatgatctccaaagctttctgtcttccaactgcagtctccacagtcttcagaagacaaatgctcaggtagtcactgtttcctttt

SEQ ID NO: 239 - DNA, 563 bp, human
aaggggatacaggccaaagaggtgctgaaaaaatatttggagtccaaggaggatgtggctgatgcacttctacagactgatcagtcactctcagaaaaggaaaaagcgattgaagtggaacgtataaaggctgaatctgcagaagctgcaaagaaaatgttggaggaaatacaaaagaagaatgaggagatgatggaacagaaagagaagagttatcaggaacatgtgaaacaattgactgagaagatggagagggacagggcccagttaatggcagagcaagagaagaccctcgctcttaaacttcaggaacaggaacgccttctcaaggagggattcgagaatgagagcaagagacttcaaaaagacatatgggatatccagatgagaagcaaatcattggagccaatatgtaacatactctaaaagtccaaggagcaaaatttgcctgtccagctccctctccccaagaaacaacatgaatgagcaacttcagagtgtcaaacaactgccattaaacttaactcaaaatcatgatgcatgcatttttgttgaaccataaa

SEQ ID NO: 240 - DNA, 541 bp, human
ggtctacgcctattacaacctggaggaaagctgtacccggttctaccatccggaaaaggaggatggaaagctgaacaagctctgccgtgatgaactgtgccgctgtgctgaggagaattgcttcatacaaaagtcggatgacaaggtcaccctggaagaacggctggacaaggcctgtgagccaggagtggactatgtgtacaagacccgactggtcaaggttcagctgtccaatgactttgacgagtacatcatggccattgagcagaccatcaagtcaggctcggatgaggtgcaggttggacagcagcgcacgttcatcagccccatcaagtgcagagaagccctgaagctggaggagaagaaacactacctcatgtggggtctctcctccgatttctggggagagaagcccaacctcagctacatcatcgggaaggacacttgggtggagcactggcctgaggaggacgaatgccaagacgaagagaaccagaaacaatgccaggacctcggcgccttcaccgagagcatggttgtc

SEQ ID NO: 241 - DNA, 591 bp, human
gaggcggagcagctgagagcctacctggagggcgagtgcgtggagtggctccgcggatacctggagaacgggaaggagacgctgcagcgcgcggaacgcccaaagacacacgtgacccaccatcccgtctctgaccatgaggccaccctgaggtgctgggccctgggcttctaccctgcggagatcacactgacctggcagcgggatggggaggaccaaactcaggacaccgagcttgtggagaccaggccagcaggagatggaaccttccagaagtgggcagctgtggtggtgccttctggacaagaacagagatacacgtgccatgtgcagcacgaggggctgcaggagccctgcaccctgagatggaagccgtcttcccagcccaccatccccaacttgggcatcgtttctggcccagctgtcctggctgtcctggctgtcctggctgtcctagctgtcctaggagctgtggtcgctgctgtgatacataggaggaagagctcaggtggaaaaggagggagctgctctcaggctgcgtccagcaacagtgcccagggctctgatgagtctctcatcgcttgtaaagcc

SEQ ID NO: 242 - DNA, 555 bp, human
  misc_feature (529)..(529): n is a, c, g, or t
ggtcttcgctggacaccatgaatcacactgtccaaaccttcttctctcctgtcaacagtggccagccccccaactatgagatgctcaaggaggagcacgaggtggctgtgctgggggcgccccacaaccctgctcccccgacgtccaccgtgatccacatccgcagcgagacctccgtgcccgaccatgtcgtctggtccctgttcaacaccctcttcatgaacccctgctgcctgggcttcatagcattcgcctactccgtgaagtctagggacaggaagatggttggcgacgtgaccggggcccaggcctatgcctccaccgccaagtgcctgaacatctgggccctgattctgggcatcctcatgaccattctgctcatcgtcatcccagtgctgatcttccaggcctatggatagatcaggaggcatcactgaggccaggagctctgcccatgacctgtatcccacgtactccaacttccattcctcgccctgcccccggagccgagtcctgtatcagccctttnatcctcacacgcttttctacaatggc

SEQ ID NO: 243 - DNA, 388 bp, human
  misc_feature (291)..(291): n is a, c, g, or t
ctcccccaaccttagatgttctaagtctttccattggatctcattggacccttccatggtgtgatcgtctgactggtgttatcaccgtgggctccctgactgggagttgatcgcctttcccaggtgctacacccttttccagctggatgagaatttgagtgctctgatccctctacagagcttccctgactcattctgaaggagccccattcctgggaaatattccctagaaacttccaaatcccctaagcagaccactgataaaaccatgtagaaaatttgttattttgnaacctcgctggactctcagtctctgagcagtgaatgattcagtgttaaatgtgatgaatactgtattttgtattgtttcaattgcatctcccagata

SEQ ID NO: 244 - DNA, 227 bp, human
ttcagaaggccagggaggacatctttatggagaccctgaacaacatcatggagtattacaacgacggtaacgatcctccctctgtggtggtcaccagccaccaggccccaggagaaaagaagaaactgaagtgcctggcctacgacttctacccagggaaaattgatgtgcactggactcgggccggcgaggtgcaggagcctgagttacggggagatgttcttcac

SEQ ID NO: 245 - DNA, 505 bp, human
ctgagagcctacctggagggcctgtgcgtggagtggctccgcagatacctggagaacgggaaggagacgctgcagcgcgcggaccccccaaagacacatgtgacccaccaccccatctctgaccatgaggccaccctgaggtgctgggccctgggcttctaccctgcggagatcacactgacctggcagcgggatggcgaggaccaaactcaggacaccgagcttgtggagaccagaccagcaggagatagaaccttccagaagtgggcagctgtggtggtgccttctggagaagagcagagatacacatgccatgtacagcatgaggggctgccgaagcccctcaccctgagatgggagccatcttcccagtccaccatccccatcgtgggcattgttgctggcctggctgtcctagcagttgtggtcatcggagctgtggtcgctactgtgatgtgtaggaggaagagctcaggtggaaaaggagggagctactctcaggctg

SEQ ID NO: 246 - DNA, 503 bp, human
tggaccgcagcggacactgcggctcagatctccaagcgcaagtgtgaggcggccaatgtggctgaacaaaggagagcctacctggagggcacgtgcgtggagtggctccacagatacctggagaacgggaaggagatgctgcagcgcgcggacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggaagcagtcttccctgcccaccatccccatcatgggtatcgttgctggcctggttgtccttgcagctgtagtcactgg

SEQ ID NO: 247 - DNA, 411 bp, human
ttctggctggaacggacgatgccccgaattcccaccctgaagaacctagaggatcttgttactgaataccacgggaacttttcggcctggagtggtgtgtctaagggactggctgagagtctgcagccagactacagtgaacgactctgcctcgtcagtgagattcccccaaaaggaggggcccttggggaggggcctggggcctccccatgcaaccagcatagcccctactgggcccccccatgttacaccctaaagcctgaaacctgaaccccaatcctctgacagaagaaccccagggtcctgtagccctaagtggtactaactttccttcattcaacccacctgcgtctcatactcacctcaccccactgtggctgatttggaattttgtgcccccatgtaagcacc

SEQ ID NO: 248 - DNA, 551 bp, human
tctgggctgggtgtgaccaagcaggatctgggcccagtccccatgtgagagcagcagaggcggtcttcaacatcctgccagccccacacagctacagctttcttgctcccttcagcccccagcccctcccccatctcccaccctgtacctcatcccatgagaccctggtgcctggctctttcgtcacccttggacaagacaaaccaagtcggaacagcagataacaatgcagcaaggccctgctgcccaatctccatctgtcaacaggggcgtgaggtcccaggaagtggccaaaagctagacagatccccgttcctgacatcacagcagcctccaacacaaggctccaagacctaggctcatggacgagatgggaaggcacagggagaagggataaccctacacccagaccccaggctggacatgctgactgtcctctcccctccagcctttggccttggcttttctagcctatttacctgcaggctgagccactctcttccctttccccagcatcactccccaaggaagagccaatgttttccacccat

SEQ ID NO: 249 - DNA, 517 bp, human
gtggcggagcagctgagagcctacctggagggcgagtgcgtggagtggctccgcagatacctggagaacgggaaggagacgctgcagcgcgcggaccccccaaagacacacgtgacccaccaccccatctctgaccatgaggccaccctgaggtgctgggccctgggcttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatgggagccgtcttcccagtccaccgtccccatcgtgggcattgttgctggcctggctgtcctagcagttgtggtcatcggagctgtggtcgctgctgtgatgtgtaggaggaagagctcaggtggaaaaggagggagctactctcaggctg

SEQ ID NO: 250 - DNA, 541 bp, human
ttgcccctgttacctaattagagcagatgacccctcccctaatgcctgcggagttgtgcacgtagtagggtcaggccacggcagcctaccggcaatttccggccaacagttaaatgagaacatgaaaacagaaaacggttaaaactgtccctttctgtgtgaagatcacgttccttcccccgcaatgtgcccccagacgcacgtgggtcttcagggggccaggtgcacagacgtccctccacgttcacccctccacccttggactttcttttcgccgtggctgcggcacccttgcgcttttgctggtcactgccatggaggcacacagctgcagagacagagaggacgtgggcggcagagaggactgttgacatccaagcttcctttgtttttttttcctgtccttctctcacctcctaaagtagacttcatttttcctaacaggattagacagtcaaggagtggcttactacatgtgggagcttttggtatgtgacatgcgggctgggcagctgttagagtccaacgtggggcagcacag

SEQ ID NO: 251 - DNA, 295 bp, human
  misc_feature (114)..(114): n is a, c, g, or t
accaatgaggttcctgaggtcacagtgttttccaagtctcccgtgacactgggtcagcccaacaccctcatctgtcttgtggacaacatctttcctcctgtggtcaacatcacntggctgagcaatgggcactcagtcacagaaggtgtttctgagaccagcttcctctccaagagtgatcattccttcttcaagatcagttacctcaccttcctcccttctgntgatgagatttatgactgcaaggtggagcactggggcctggatgagcctcttctgaaacactgggagcctg

SEQ ID NO: 252 - DNA, 434 bp, human
caccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggaagcagtcttccctgcccaccatccccatcatgggtatcgttgctggcctggttgtccttgcagctgtagtcactggagctgcggtcgctgctgtgctgtggagaaagaagagctcagattgaaaaggagggagctactctcaggctgcaatgtgaaacagctgccctgtgtgggactgagt

SEQ ID NO: 253 - DNA, 285 bp, human
caatctttcatgtcttgtcatacaaatttataaaggtctgcactcctttatctgtaattgtaattccaaaatccaaaaagctctgaaaacaaggtttccataagcttggtgacaaaattcatttgcttgcaatctaatctgaactgaccttgaatctttttatcccatttagtgtgaatattcctttattttgctgcttgatgatgagagggagggctgctgccacagactgtggtgagggctggttaatgtagtatggtatatgcacaaaactacttttctaaa

SEQ ID NO: 254 - DNA, 482 bp, human
atttctttggcagttttcgtgctaatgtttttgctaaggaagataagctctgaaccattaaaggacgagtttaaaaacacaggatcaggtctcctgggcatggctaacattgacctggaaaagagcaggactggtgatgaaattattcttccgagaggcctcgagtacacggtggaagaatgcacctgtgaagactgcatcaagagcaaaccgaaggtcgactctgaccattgctttccactcccagctatggaggaaggcgcaaccattcttgtcaccacgaaaacgaatgactattgcaagagcctgccagctgctttgagtgctacggagatagagaaatcaatttctgctaggtaattaaccatttcgactcgagcagtgccactttaaaaatcttttgtcagaatagatgatgtgtcagatctctttaggatgactgtatttttcagttgccgatacagctttttgtcctctaactg

SEQ ID NO: 255 - DNA, 378 bp, human
acatctccattacaaatgccacagttgaagacagtggaacctactactgtacgggcaaagtgtggcagctggactatgagtctgagcccctcaacattactgtaataaaagctccgcgtgagaagtactggctacaattttttatcccattgttggtggtgattctgtttgctgtggacacaggattatttatctcaactcagcagcaggtcacatttctcttgaagattaagagaaccaggaaaggcttcagacttctgaacccacatcctaagccaaaccccaaaaacaactgatataattactcaagaaatatttgcaacattagtttttttccagcatcagcaattgctactcaattgtcaaacacagcttgca

SEQ ID NO: 256 - DNA, 503 bp, human
cagatcctccaaaggcacacgttgcccaccaccccatctctgaccatgaggccaccctgaggtgctgggccctgggcttctaccctgcggagatcacgctgacctggcagcgggatggggaggaacagacccaggacacagagcttgtggagaccaggcctgcaggggatggaaccttccagaagtgggccgctgtggtggtgccttctggagaggaacagagatacacatgccatgtgcagcacgaggggctgccccagcccctcatcctgagatgggagcagtctccccagcccaccatccccatcgtgggcatcgttgctggccttgttgtccttggagctgtggtcactggagctgtggtcgctgctgtgatgtggaggaagaagagctcagatagaaacagagggagctactctcaggctgcagtcactgacagtgcccagggctctggggtgtctctcacagctaataaagtgtgagacagcttccttgtgtgggac

SEQ ID NO: 257 - DNA, 531 bp, human
  misc_feature (35)..(35): n is a, c, g, or t
ggaggacataacactggatcgtctaggagagaccntttgaattcccttngactncccacaaanttttcagtaaaaacctccttttgtctgacataagtcnacataataaagggaagtgctgtatggggaaattattttagcatacttatttctaaatcctctaaagaccctgaggacatgtgatgcatagnttttattggtggagatttgagaagaaatgacctgtgtgtaggccctttacacnaatntcatggacaggacaagtagccaagcgccttttgtggtggaaataatttgggatccatntgataaagatgggcaatctctgaagaaagtgtcacnatttcataagagacatggcctgggcacaatgttaacaaaacttcctatttttgcccccaccccatagtagctctgcagtcacaatgtgcacttacgttgggtgtccccagccaaagccagtggggagctcagcaccgtcaatgtcactgtcagcgctgccatacaggagcctccagggagcttcagaca

SEQ ID NO: 258 - DNA, 465 bp, human
gttttggaccaacaagtgcctcctttggtgaaggtgacacatcatgtgacctcttcagtgaccactctacggtgtcgggccttgaactactacccccagaacatcaccatgaagtggctgaaggataagcagccaatggatgccaaggagttcgaacctaaagacgtattgcccaatggggatgggacctaccagggctggataaccttggctgtaccccctggggaagagcagagatatacgtgccaggtggagcacccaggcctggatcagcccctcattgtgatctgggagccctcaccgtctggcaccctagtcattggagtcatcagtggaattgctgtttttgtcgtcatcttgttcattggaattttgttcataatattaaggaagaggcagggttcaagaggagccatggggcactacgtcttagctgaacgtgagtgacacgcagcctgcagactc

SEQ ID NO: 259 - DNA, 436 bp, human
gctggaatacttactcttgtcgggagattgaaccactaaaatgttagagcagaattcattatgctgtggtcacaggggtgtcttgtctgagaacaaatacaattcagtcttctctttggggttttagtatgtgtcaaacataggactggaagtttgcccctgttcttttttcttttgaaagaacatcagttcatgcctgaggcatgagtgactgtgcatttgagatagttttccctattctgtggatacagtcccagagttttcagggagtacacaggtagattagtttgaagcattgaccttttatttattccttatttctctttcatcaaaacaaaacagcagctgtgggaggagaaatgagagggcttaaatgaaatttaaaataagctatattatacaaatactatctctgtattgttctgaccctggtaaa

SEQ ID NO: 260 - DNA, 551 bp, human
  misc_feature (158)..(158): n is a, c, g, or t
agtcagtgttgtcttaatatccttgataatgctgtaaagtttatttttacaaatatttctgtttaagctatttcacctttgtttggaaatccttcccttttaaagagaaaatgtgacacttgtgaaaaggcttgtaggaaagctcctccctttttttnctttaaacctttaaatgacaaacctaggtaattaatggttgtgaatttctatttttgctttgtttttaatgaacatttgtctttcagaataggattctgtgataatatttaaatggcaaaaacaaaacataattttgtgcaattaacaaagctactgcaagaaaaataaaacatttcttggtaaaaacgtatgtatttatatattatatatttatatataatatatattatatatttagcattgctgagctttttagatgcctattgtgtatcttttaaaggttttgaccattttgttatgagtaattacatatatattacattcactatattaaaattgtacttttttactatgtgnctcattggttcatagtctttattttgtcctttgaa

SEQ ID NO: 261 - DNA, 522 bp, human
tgtgggcattgtgggctacgtggaaacccctcgaggcctccggaccttcaagactgtctttgctgagcacatcagtgatgaatgcaagaggcgtttctataagaattggcataaatctaagaagaaggcctttaccaagtactgcaagaaatggcaggatgaggatggcaagaagcagctggagaaggacttcagcagcatgaagaagtactgccaagtcatccgtgtcattgcccacacccagatgcgcctgcttcctctgcgccagaagaaggcccacctgatggagatccaggtgaacggaggcactgtggccgagaagctggactgggcccgcgagaggcttgagcagcaggtacctgtgaaccaagtgtttgggcaggatgagatgatcgacgtcatcggggtgaccaagggcaaaggctacaaaggggtcaccagtcgttggcacaccaagaagctgccccgcaagacccaccgaggcctgcgcaaggtggcctgtattggggcatggcatcctgt

SEQ ID NO: 262 - DNA, 305 bp, human
aaatctcgtctcgaccaagagctgaagctgatcggcgagtatgggctccggaacaaacgtgaggtctggagggtcaaatttaccctggccaagatccgcaaggccgcccgggaactgctgacgcttgatgagaaggacccacggcgtctgttcgaaggcaacgccctgctgcggcggctggtccgattgggggtgctggatgagggcaagatgaagctggattacatcctgggcctgaagatagaggatttcttagagagacgcctgcagacccaggtcttcaagctgggcttggccaagtccat

SEQ ID NO: 263 - DNA, 277 bp, human
gtggaggtgactggtgatgaatacaatgtggaaagcattgatggtcagccaggtgccttcacctgctatttggatgcaggccttgccagaactaccactggcaataaagtttttggtgccctgaagggagctgtggatggaggcttgtctatccctcacagtaccaaacgattccctggttatgattctgaaagcaaggaatttaatgcagaagtacatcggaagcacatcatgggccagaatgttgcagattacatgcgctacttaatggaagaag

SEQ ID NO: 264 - DNA, 472 bp, human
acttcgtactttctatgagaagcgtatggccacagaagttgctgctgacgctctgggtgaagaatggaagggttatgtggtccgaatcagtggtgggaacgacaaacaaggtttccccatgaagcagggtgtcttgacccatggccgtgtccgcctgctactgagtaaggggcattcctgttacagaccaaggagaactggagaaagaaagagaaaatcagttcgtggttgcattgtggatgcaaatctgagcgttctcaacttggttattgtaaaaaaaggagagaaggatattcctggactgactgatactacagtgcctcgccgcctgggccccaaaagagctagcagaatccgcaaacttttcaatctctctaaagaagatgatgtccgccagtatgttgtaagaaagcccttaaataaagaaggtaagaaacctaggaccaaagcacccaagattcagcgtcttgtt

SEQ ID NO: 265 - DNA, 405 bp, human
  misc_feature (165)..(165): n is a, c, g, or t
ttgatcttggtttgcaagcccttctcccatcagtcctagattaggccctgttcagccatgcaggggtgttggtttatgcgtgctgcagcagtgggcataatgaatataatttacccagtggacaaaggtgtgtaccaagtgaatttaaataattggtgtggattnggccagtagctaagnnntgggcttttaaagagtattgaagattgaanagggtttttctttcttttttaaaaaagaaaaacaaactattgattgtagataatgaaaagctagggtttgccctcttcatgtctactctccttccaaatagttatatccaaaactgtttttccctctcccctaccttgtcccccctattaaaatagaaacagggattgattaatgtcccgctcctgaatacat

SEQ ID NO: 266 - DNA, 482 bp, human
cctccctcacagtgcagggcggtatgccgcaaacgctttccgcaaagctcagtgtcccattgtggagcgcctcactaactccatgatgatgcacggccgcaacaacggcaagaagctcatgactgtgcgcatcgtcaagcatgccttcgagatcatacacctgctcacgggcgagaaccctctgcaggtcctggtgaacgccatcatcaacagtggtccccgggaggactccacacgcattgggcgcgccgggactgtgagacgacaggctgtggatgtgtcccccctgcgccgtgtgaaccaggccatctggctgctgtgcacaggcgctcgtgaggctgccttccggaacattaagaccattgctgagtgcctggcagatgagctcatcaatgctgccaagggctcctcgaactcctatgccattaagaagaaggacgagctggagcgtgtggccaagtccaaccgctgattttccagct

SEQ ID NO: 267 - DNA, 499 bp, human
gatacgctggggcccatgcagaaggagctggccgagcagctgggcctgtctactggcgagaaggagaagctgccgggagagctagagccggtgcaggccacgcagaacaagacagggaagtatgtgccgccgagcctgcgcgacggggccagccgccgcggggagtccatgcagcccaaccgcagagccgacgacaacgccaccatccgtgtcaccaacttgtcagaggacacgcgtgagaccgacctgcaggagctcttccggcctttcggctccatctcccgcatctacctggctaaggacaagaccactggccaatccaagggctttgccttcatcagcttccaccgccgcgaggatgctgcgcgtgccattgccggggtgtccggctttggctacgaccacctcatcctcaacgtcgagtgggccaagccgtccaccaactaagccagctgccactgtgtactcggtccgggacccttggcgacagaagacagcc

SEQ ID NO: 268 - DNA, 153 bp, human
ggaactatgtctggaaggctgtggtccaaggccatttttgctggctataagcggggtctccggaaccaaagggagcacacagctcttcttaaaattgaaggtgtttacgcccgagatgaaacagaattctatttgggcaagagatgcgcttat

SEQ ID NO: 269 - DNA, 324 bp, human
cagcttgaagaactatgatccccagaaggacaagcgcttctcgggcaccgtcaggcttaagtccactccccgccctaagttctctgtgtgtgtcctgggggaccagcagcactgtgacgaggctaaggccgtggatatcccccacatggacatcgaggcgctgaaaaaactcaacaagaataaaaaactggtcaagaagctggtcaagaagtatgatgcgtttttggcctcagagtctctgatcaagcagattccacgaatcctcggcccaggtttaaataaggcaggaaagttcccttccctgctcacacacaacgaaaacat

SEQ ID NO: 270 - DNA, 450 bp, human
tcctgaggaacatgcgctttgccaagaagcacaacaaaaagggcctaaagaagatgcaggccaacaatgccaaggccatgagtgcacgtgccgaggctatcaaggccctcgtaaagcccaaggaggttaagcccaagatcccaaagggtgtcagccgcaagctcgatcgacttgcctacattgcccaccccaagcttgggaagcgtgctcgtgcccgtattgccaaggggctcaggctgtgccggccaaaggccaaggccaaggccaaggccaaggatcaaaccaaggcccaggctgcagccccagcttcagttccagctcaggctcccaaacgtacccaggcccctacaaaggcttcagagtagatatctctgccaacatgaggacagaaggactggtgcgaccccccacccccgcccctgggctaccatctgcatggggctggggtcc

SEQ ID NO: 271 - DNA, 525 bp, human
aattctgatgtcgtacctaaggcttgtccatctttgttgttggaggtgccatggctcctgtgaaaaagcttgtggtgaaggggggcaaaaaaaagaagcaagttctgaagttcactcttgattgcacccaccctgtagaagatggaatcatggatgctgccaattttgagcagtttttgcaagaaaggatcaaagtgaacggaaaagctgggaaccttggtggaggggtggtgaccatcgaaaggagcaagagcaagatcaccgtgacatccgaggtgcctttctccaaaaggtatttgaaatatctcaccaaaaaatatttgaagaagaataatctacgtgactggttgcgcgtagttgctaacagcaaagagagttacgaattacgttacttccagattaaccaggacgaagaagaggaggaagacgaggattaaatttcatttatctggaaaattttgtatgagttcttgaataaaacttgggaaccaaaatggtggtttatccttgtatctctgcagtgtg

SEQ ID NO: 272 - DNA, 418 bp, human
gccgcttccctcaagagtaactacaatcttcccatgcacaagatgattaatacagatcttagcagaatcttgaaaagcccagagatccaaagagcccttcgagcaccacgcaagaagatccatcgcagagtcctaaagaagaacccactgaaaaacttgagaatcatgttgaagctaaacccatatgcaaagaccatgcgccggaacaccattcttcgccaggccaggaatcacaagctccgggtggataaggcagctgctgcagcagcggcactacaagccaaatcagatgagaaggcggcggttgcaggcaagaagcctgtggtaggtaagaaaggaaagaaggctgctgttggtgttaagaagcagaagaagcctctggtgggaaaaaaggcagcagctaccaagaaaccagccc

SEQ ID NO: 273 - DNA, 454 bp, human
  misc_feature (219)..(220): n is a, c, g, or t
cgctggaagccgtgcttcggtatcttgagacccacccccgcccccccaagcctgaccccgtgaaaagcgtgtccagcgtgctcagcagcctgacgcccgccaaggtggcccccgtcatcaacaacggctcccccaccatcctgggcaagcgcagctacgagcagcacaacggggtggacggcaacatgaagaagcgcctcttgatgcccagtaggggtnncntggncnnnnnaaaccacggacagaccaggcacatgggaccaagccggaacctcctgctcaacgggaagtcctaccccaccaaagtgcgcctgatccgggggggctccctgcccccagtcaagcggcggcggatgaactggatcgacgccccggatgacgtgttctacatggccacagaggagaccaggaagatccgcaagctgctctcatcctcggaaaccaagcgtgctgc

SEQ ID NO: 274 - DNA, 531 bp, human
ggcactgtaatggcgttgactggcgtcagaagctggactctcagcgaggggctgtcattgccacggagctgaagaacaacagctacaagttggcccggtggacctgctgtgctttgctggctggatctgagtacctcaagcttggttatgtgtctcggtaccacgtgaaagactcctcacgccacgtcatcctaggcacccagcagttcaagcctaatgagtttgccagccagatcaacctgagcgtggagaatgcctggggcattttacgctgcgtcattgacatctgcatgaagctggaggagggcaaatacctcatcctcaaggaccccaacaagcaggtcatccgtgtctacagcctccctgatggcaccttcagctctgatgaagatgaggaggaagaggaggaggaagaagaggaagaagaagaggaagaaacttaaaccagtgatgtggagctggagtttgtccttccaccgagactacgagggcctttgatgcttagtggaatgtgtgtctaacttgctctct

SEQ ID NO: 275 - DNA, 365 bp, human
atcttttgtctttccgtggagctgtcgccatgaaggtcgagctgtgcagttttagcgggtacaagatctaccccggacacgggaggcgctacgccaggaccgacgggaaggttttccagtttcttaatgcgaaatgcgagtcggctttcctttccaagaggaatcctcggcagataaactggactgtcctctacagaaggaagcacaaaaagggacagtcggaagaaattcaaaagaaaagaacccgccgagcagtcaaattccagagggccattactggtgcatctcttgctgatataatggccaagaggaatcagaaacctgaagttagaaaggctcaacgagaacaagctatcagggctgct

SEQ ID NO: 276 - DNA, 465 bp, human
tgaaagactctggtgccttgtggtgcccttctgaaattcaaacagtaatgcaaaagtgtctgcattagaatttacggtgtctaaaattcatgtttttaaaagagcttgcctacagatggtttccacacttgaaattgtgccctgcgagttgcatagctggaagttcaatgctcagtcctaccttggctcccattaaacatttggtgctctgtggattgagttgaacgtgttgaggctttgcaatttcacttgtgttaaaggctctggcatttttccatttctatgcaaatttctttgaagcagaattgcttgcatatttcttctctgccgtcacagaaagcagagtttctttcaaacttcactgaggcatcagttgctctttggcaatgtcccttaaccatgattattaactaagtttgtggcttgagtttacaaattctacttgttgcattgatgttcccatgt

SEQ ID NO: 277 - DNA, 133 bp, human
ggtgcaggtcttcggacgcaagaagacagcgacagctgtggcgcactgcaaacgcggcaatggtctcatcaaggtgaacgggcggcccctggagatgattgagccgcgcacgctacagtacaagctgctggag

SEQ ID NO: 278 - DNA, 477 bp, human
  misc_feature (78)..(78): n is a, c, g, or t
gtagtatctaaatccagctttttcttacctctggtacacttcagattacttacctaatacatattgagggcttttaanccttagttttatcaatttttctagttttattttatttttataaaataattgaaaaagaaccaaaaatgaattcagaagttccatagatggatttttgccccttcttaattttctcccacaatgaaaacattttagaagacattttgaatcaaaatgtattatgaccattgaaagttctctattttctaagaagctgagcagctccaagggcctcgtttcaggtggtactctatgtattgtctgtgggcttgtttctacctgtacagtggaaccatgtgaccatgtcttgtgcttgcaatatagaaaccatgcagtgctgttttgtgaattggcagtttctgtataaactcttatttatatacattggcctctctgtatgattttccatttgctacatga

SEQ ID NO: 279 - DNA, 391 bp, human
tcagaacagggctttggtcgatggaccttgcactcaagtgaggagacaggccatgcctttcaagtgcatgcagctcactgatttcatcctcaagtttccgcacagtgcccaccagaagtatgtccgacaagcctggcagaaggcagacatcaatacaaaatgggcagccacacgatgggccaagaagattgaagccagagaaaggaaagccaagatgacagattttgatcgttttaaagttatgaaggcaaagaaaatgaggaacagaataatcaagaatgaagttaagaagcttcaaaaggcagctctcctgaaagcttctcccaaaaaagcacctggtactaagggtactgctgctgctgctgctgctgctgctaaagttccagcaaaa

SEQ ID NO: 280 - DNA, 432 bp, human
gaacgcggagagcacgccatgaaggcctcgggcacgctacgagagtacaaggtagtgggtcgctgcctgcccacccccaaatgccacacgccgcccctctaccgcatgcgaatctttgcgcctaatcatgtcgtcgccaagtcccgcttctggtactttgtatctcagttaaagaagatgaagaagtcttcaggggagattgtctactgtgggcaggtgtttgagaagtcccccctgcgggtgaagaacttcgggatctggctgcgctatgactcccggagcggcacccacaacatgtaccgggaataccgggacctgaccaccgcaggcgctgtcacccagtgctaccgagacatgggtgcccggcaccgcgcccgagcccactccattcagatcatgaaggtggaggagatcgcggccagcaagtgccgc

SEQ ID NO: 281 - DNA, 477 bp, human
gtggttcaagttatccggcagcgaaactgggtggtcgtgggagggctgaacacacattaccgctacattggcaagaccatggattaccggggaaccatggtccctagtgaagcccccttgctccaccgccatgtcaaacttgtggatcctatggacaggaaacccactgagatcgactggagatttactgaagcaggacagcgggtaagagtctccacacgatcagggagaattatccctaaacccgaatttcccagagctgatggcatcgtccctgaaacgtggattgatggccccaaagacacatcagtggaagatgctttagaaagaacctatgtgccctgtctaaagacactgcaggaggaggtgatggaggccatggggatcaaggagacccggaaatacaagaaggtctattggtattgagcctggggcagagcagctcctccccaacttctgtcccagccttgaaggctg

SEQ ID NO: 282 - DNA, 233 bp, human
gttgaattcttcatttttgccatctttaactgtcatcactggggcagggaagtcctgttccagaagtaccaggctgtagatttgataagctagatgcagtagaccgaaaccatccaaaacctgtttagcttcttcctccattggagtttattgggacaaacaggagagccagccattgtctccagtacttgcctcattctcatcatccaaactgaacatttgtatcccaagca

SEQ ID NO: 283 - DNA, 545 bp, human
aagcagggtgtcttgacccatggccgtgtccgcctgctactgagtaaggggcattcctgttacagaccaaggagaactggagaaagaaagagaaaatcagttcgtggttgcattgtggatgcaaatctgagcgttctcaacttggttattgtaaaaaaaggagagaaggatattcctggactgactgatactacagtgcctcgccgcctgggccccaaaagagctagcagaatccgcaaacgtttcaatctctctaaagaagatgatgtccgccagtatgttgtaagaaagcccttaaataaagaaggtaagaaacctaggaccaaagcacccaagattcagcgtcttgttactccacgtgtcctgcagcacaaacggcggcgtattgctctgaagcaacagcgtaccaagaaaaataaagaagaggctgcagaatatgctaaacttttggccaagagaatgaaggaggctaaggagaagcgccaggaacaaattgcgaagagacgcagactttcctctctgcgagcttctacttctaagtctga

SEQ ID NO: 284 - DNA, 434 bp, human
cggcacttggcgtaaagccgcttccctcaagagtaactacaatcttcccatgcacaagatgattaatacagatcttagcagaatcttgaaaagcccagagatccaaagagcccttcgagcaccacgcaagaagatccatcgcagagtcctaaagaagaacccactgaaaaacttgagaatcatgttgaagctaaacccatatgcaaagaccatgcgccggaacaccattcttcgccaggccaggaatcacaagctccgggtggataaggcagctgctgcagcagcggcactacaagccaaatcagatgagaaggcggcggttgcaggcaagaagcctgtggtaggtaagaaaggaaagaaggctgctgttggtgttaagaagcagaagaagcctctggtgggaaaaaaggcagcagctaccaagaaaccagccc

SEQ ID NO: 285 - DNA, 370 bp, human
gtgaaaaggagaaccccatgcgggaacttcgcatccgcaaactctgtctcaacatctgtgttggggagagtggaggcagactgacgcgagcagccaaggtgttggagcagctcacagggcagacccctgtgttttccaaagctagatacactgtcagatcctttggcatccggagaaatgaaaagattgctgtccactgcgcagttcgaggggccaaggcagaagaaatcttggagaagggtctaaaggtgcgggagttggagttaagaaaaaacaacttctcagatactggaaactttggttttgggatccaggaacacattgatctgggtatcgaatatgacccaagcattggtatctacggcctgga

SEQ ID NO: 286 - DNA, 525 bp, human
gcagatgctgggagaacgcttgttcccactcatccaaacaatgcattcaaatctggctgggaagatcacgggaatgctgctggagatagacaactctgagctgctgcacatgttagagtcccccgagtctctccgctccaaggtggatgaagctgtagcagttctacaggctcatcatgccaagaaagaagctgcccagaaggtgggcgctgttgctgctgctacctcttagacaaggaaaaaccgattcaaaagccaaataaccccttatggaattcaactcaaggtttgaagacttcctagcttgtcctatggacctcaacaccaaggattacaaattgcaaatttaataggtcattttgtatcaaaaggtcaattatgaagcacctagaatttttcaattatacgaatatgttctttgggttctgctgtggcccagacagtgttaactttttttttattgtgggttttgattttttcccccagaaattggttttatttgatgtacccaagtcttacgtttcc

SEQ ID NO: 287 - DNA, 433 bp, human
gaaaggttcggcgcaaggagcccaagagccaggatatctacctgaggctgttggtcaagttatacaggtttctggccagaagaaccaactccacattcaaccaggttgtgttgaagaggttgtttatgagtcgcaccaaccggccgcctctgtccctttcccggatgatccggaagatgaagcttcctggccgggaaaacaagacggccgtggttgtggggaccataactgatgatgtgcgggttcaggaggtacccaaactgaaggtatgtgcactgcgcgtgaccagccgggcccgcagccgcatcctcagggcagggggcaagatcctcactttcgaccagctggccctggactcccctaagggctgtggcactgtcctgctctccggtcctcgcaagggccgagaggtgtaccggcatttcggcaaggc

SEQ ID NO: 288 - DNA, 569 bp, human
  misc_feature (93)..(93): n is a, c, g, or t
gaaactgttcaatcttaacacattgttactttaatatatgtataaagaagtattactgtttgtaaagctgctgtttgcttaaaaaaaaaaaancacccttgtcatgtattttctgtatgttgggccaacaggttagaacatcaactcatttaaaaatttttatctttttttgatttaaaaaaattctgtgaaataatttatttacagacatcttcctcctccctcatcccttccaacctttacatacatcacagaatcaaccaaactgtttgcctaatctgaaatctgaatcctaatgagaaaaatttaaattttgttggcacatcacaccttgaaagtatttgtattattttataatttaatttctaaatataccacataagtttataatttaatgtcttaattgtaatgctctaataaaaaactagcaaaattagtgtgagttataacatgaagggattttcatcttttgctgtatgaaggataattgttatatcacatttggggggtaataacagcttttttgcactatgtaaatactagtggggattcttctgtactaataaaat

SEQ ID NO: 289 - DNA, 432 bp, human
  misc_feature (251)..(251): n is a, c, g, or t
ccgcacgatgcctggagttactgtaaaagacgtgaaccagcaggagttcgtcagagctctggcagccttcctcaaaaagtccgggaagctgaaagtccccgaatgggtggataccgtcaagctggccaagcacaaagagcttgctccctacgatgagaactggttctacacgcgagctgcttccacagcgcggcacctgtacctccggggtggcgctggggttggctccatgaccaagatctatgggggangtcagagaancggcgtcatgcccagccacttcanccgaggctccnaagagtgtgngcccnccnngggtcctccaagccctggaggggctgaaaatggtggaaaaggaccaagatggcggccgcaaactgacacctcagggacaaagagatctggacagaatcgccggacaggtggcagctg

SEQ ID NO: 290 - DNA, 471 bp, human
  misc_feature (153)..(161): n is a, c, g, or t
tgctttggagctatcagatttgcatgttagagaaatgactctggtctcagtggagagagtggactgaagagggcaagaatggaagccgggaagagtgaacagaagaaatcttgggctttgattgggtcggaaagaaagtgtggccctacccannnnnnnnnacaagatnngcaancccnncactnncccttgcaaggnnnacaggncgctgcggctctgtgntggtacgcctcatccctgcancccnnggggcactggcatcgtctccgcacctgtgcctaagaagctgctcatgatggctggtatcgatgactgctacancntcagcccggggctgcactgccaccctgggcaacttcnncaaggccacctttgatgccatttctaagacctacagctacctgacccccgacctctggaaggagactgtattcaccaagtctccctatcaggagttcactgaccacctcg

SEQ ID NO: 291 - DNA, 488 bp, human
  misc_feature (212)..(212): n is a, c, g, or t
cagcccctaaaggtaatgccagcccctccaccaaaggagaatgcttgggtgaagcgaagttctaaccctcctgctcgatctcagagctcagacacagagcagcagtcccctacaagtggtgggggaaaagtagctccagctcaaccatctgaggaaggaccaggaaggaaagatgaaaataaagtagatgggatgaatgccccaaaaggccnaaactgggaactctagccgtggtccaggagacggagggaacagagaccactggaaggagtcagataggaaagatggcaaaaaggatcaagactccagatctgcacctgagccaaagaaacctgaggaaaatccagcttctaagttcagttctgcaagcaagtatgctgctctctctgttgatggtgaagatgaaaatgagggagaagattatgccgaatagacctctacatcctgtgcttttctcctagtttctctccaccctggaacattcgaga

SEQ ID NO: 292 - DNA, 545 bp, human
ggtccttatgttgtcaccatagagcaacaaaggtatagggctgccttcctcttatttatttggggacattattttgttatttagataccaaggcctaattaattaagtacctataagaactatttatttggagtaactgagcctgtaactcaggtttatggctgttaagtatagattggggaatctttattatgtcttctcctaagcagtttaaccaaatgtgtggttagtgtttttttattcccctaagacagaaagaacaaaaaatgttttaaatttctcttatataggaaataataggaacgtcaaagctctgtatacctactaagtggaaaacaagaccatcatctaagtgatttgagaaattaacaaaagtagtgactacacagcaataattacagtaaattaaataaagattcctttaaggcagacaagggctaagatttccttagcagtaataatgacatacactgaattgaaaatctattttattacagaaagatcagtttctaacaaatgaaaatgtatcacctgttccttaactg

SEQ ID NO: 293 - DNA, 497 bp, human
  misc_feature (355)..(355): n is a, c, g, or t
ccatcttccagtaattcgccaaaatgacgaacacaaagggaaagaggagaggcacccgatatatgttctctaggccttttagaaaacatggagttgttcctttggccacatatatgcgaatctataagaaaggtgatattgtagacatcaagggaatgggtactgttcaaaaaggaatgccccacaagtgttaccatggcaaaactggaagagtctacaatgttacccagcatgctgctggcattgttgtaaacgaacaagttaagggcaagattcttgccaagagaattaatgtgcgtattgagcacattaagcactctaagagccgagataacttcctgaaacgtgtgaaggnagatgatcaggaaaagnaagaagcccaagagaaaggtacctgggttcaactaaagcgccancctgctccacccagagaagcacactttgtgagaaccaatgggaaggagcctgagctgctggaacctattccctatgaattc

SEQ ID NO: 294 - DNA, 259 bp, human
acacagtccgtgtgcggggaggtaacaagaaataccgtgccctgaggttggacgtggggaatttctcctggggctcagagtgttgtactcgtaaaacaaggatcatcgatgttgtctacaatgcatctaataacgagctggttcgtaccaagaccctggtgaagaattgcatcgtgctcatcgacagcacaccgtaccgacagtggtacgagtcccactatgcgctgcccctgggccgcaagaagggagccaagctgac

SEQ ID NO: 295 - DNA, 177 bp, human
tccgcctgctactgagtaaggggcattcctgttacagaccaaggagaactggagaaagaaagagaaaatcagttcgtggttgcattgtggatgcaaatctgagcgttctcaacttggttattgtaaaaaaaggagagaaggatattcctggactgactgatactacagtgcctcgcc

SEQ ID NO: 296 - DNA, 242 bp, human
ccattccccagtggattcggatgaaaactggaaataaaatcaggtacaactccaaaaggagacattggagaagaaccaagctgggtctataaggaattgcacatgagatggcacacatatttatgctgtctgaaggtcacgatcatgttaccatatcaagctgaaaatgtcaccactatctggagatttcgacgtgttttcctctctgaatctgttatgaacacgttggttggctggattca

SEQ ID NO: 297 - DNA, 510 bp, human
tggaattggaggccgggcgcagtggctcacgcctgtaatcccagcactttgggaggccaaggtgggtggatcacctgaggtcaggagctcgagaccagcctggccaacatggtgaaaccccatctctactaaaaatacaaaaattagccaggtgtgatggtgcatgcctgtaatcccagctcctcagtaggctgagacaggagcatcacttgaacgtgggaggcagaggttgcagtgagccgagattgcaccaccgcactccagcctgggtgacagagcgagacttatctcataaataaatagatagatactccagcctgggtgacagagcgagacttatagatagatagatagatagatggatagatagatagatagatagatagatagataaacggaattggagccattttgctttaagtgaatggcagtcccttgtcttattcagaatataaaattcagtctgaatggcatcttacagattttacttcaatttttgtgtacggtatt

SEQ ID NO: 298 - DNA, 378 bp, human
gctactttgaattaatctgcctttatgtttgggagaagaaagctgagacattgcatgaaagatgatgagagataaatgttgatcttttggccccatttgttaattgtattcagtatttgaacgtcgtcctgtttattgttagttttcttcatcatttattgtatagacaatttttaaatctctgtaatatgatacattttcctatcttttaagttattgttacctaaagttaatccagattatatggtccttatatgtgtacaacattaaaatgaaaggctttgtcttgcattgtgaggtacaggcggaagttggaatcaggttttaggattctgtctctcattagctgaataatgtgaggattaacttctgccagct

SEQ ID NO: 299 - DNA, 560 bp, human
cagcctctggagagtactcagtctcaggacttctagagaaaggcctggtgcaggcggcttgctgggggatgtgagcgctcaggacgtgatgaggtactcgtggttctggagctctagaaacacttctgatgcatgaaaaatgtgtgatggtgcaaggaatggattcaggatgttgttggagaaacaagtttgtgattagtccttaaaacttagctccctgggacattcttcaattccacatctgtttctagaaaccagccctttttccccccacttttgagaaataaaaaagccttaggtaaataagtcattctccctagcagagccacttgggtctcctgcatggaagccatcacacttgggcaggtgttcagtgactggtaggtgtagatacagcaggagtggccatgtggtccacggctttttaccccttcttgatcctcatttcttgggctgaatttagactctctcacagaggtggctcacagagaaggatggcagatggtgcagccaacaatgctgaccggtgcttatcctctaagccctgatccacaataaaa

SEQ ID NO: 300 - DNA, 375 bp, human
ttttaccttggatgctgacttctaaatgaactgaagatgtgcccttacttggctgattttttttttccatctcataagaaaaatcagctgaagtgttaccaactagccacaccatgaattgtccgtaatgttcattaacagcatctttaaaactgtgtagctacctcacaaccagtcctgtctgtttatagtgctggtagtatcaccttttgccagaaggcctggctggctgtgacttaccatagcagtgacaatggcagtcttggctttaaagtgaggggtgaccctttagtgagcttagcacagcgggattaaacagtcctttaaccagcacagccagttaaaagatgcagcctcactgcttcaacgcagatt

SEQ ID NO: 301 - DNA, 229 bp, human
  misc_feature (146)..(147): n is a, c, g, or t
gccaaactcattgccatgacaactctttgtactgtgtccgtgccacagatctgttggtcacattgttaatagtaaaggggacaagttggagacggtcaatttttacattttttgttgcaattttttcttcaatggttgtaagtagnnttttttttttttttaataataaaagggttcactagttaatactctagaaatatctgtgtgttgcaattcaaatgtatgttga

SEQ ID NO: 302 - DNA, 445 bp, human
ggacactaagtccattgttacatgaaagtgatagatagcaacaagttttggagaagagagagggagataaaagggggagacaaaagatgtacagaaatgatttcctggctggccaactggtggccagtgggaggtgatggtggacctagactgtgcttttctgtcttgttcagccttgacccaccttgagagagagccaccaggaaggcgcatcttagcagatgggaggaactgctgagagaagatgggcagaaagctggagcccctggagttggctgtgtctgtgtttgtgactgattactggctgtgtcttgggtgggcagaaactcgaacttgctatgtaatttgtgtctagttattcagaggagtaagatggtgatgttcacctggcaatcagctgagttgagactttggaataagacactggttttcatgcgctgttttt

SEQ ID NO: 303 - DNA, 468 bp, human
ttgtgttttgtcattagctctgccctttttaattaaatattttggttcatggaccaaagggtttacttgacaaatttgtgtgacagactccgaacaattcctttactacgaagtataatttataaaataaaatatacccattttaagggtacagtttgatttttgaccagtgaaactatgatcccaatcaaggtatagatgccgtcaccccaaaaagttccctccatatccctttgcagtcagttcatccctaccctggcccagatgatcactgatcttgtcattatagatgagttttgccagttcaagaatttaatggaatcagatattgtaagcattcttgtgtaatacttcattctctctcattattgagattcatccatattgttgaatgtttcactagttaatgtttattgttcaatatttttgtatatacttttaaagcctattcacttgctgatggatctt

SEQ ID NO: 304 - DNA, 577 bp, human
gatgaacctattctgaagcacttgaaagatattaaagtgaagttctcagatgctggccagcctatgagttttgtcttagaatttcactttgaacccaatgaatattttacaaatgaagtgctgacaaagacatacaggatgaggtcagaaccagatgattctgatcccttttcttttgatggaccagaaattatgggttgtacagggtgccagatagattggaaaaaaggaaagaatgtcactttgaaaactattaagaagaagcagaaacacaagggacgtgggacagttcgtactgtgactaaaacagtttccaatgactctttctttaacttttttgcccctcctgaagttcctgagagtggagatctggatgatgatgctgaagctatccttgctgcagacttcgaaattggtcactttttacgtgagcgtataatcccaagatcagtgttatattttactggagaagctattgaagatgatgatgatgattatgatgaagaaggtgaagaagcggatgaggaaggggaagaagaaggagatgaggaaaatgatccagactatgacccaaaga

SEQ ID NO: 305 - DNA, 523 bp, human
tttctctcgttttccttagttatcagtcctgtcctgtcccactcaggtctgtacttagggcagctggcctggatgggcttcactggggccctgtctgtgtgctgagccagtttcccctgctggctgcaagctgtgggttctttctcctctgtgcccctcatgctgatcttctagatgccactcccaaatccccttcatacccaccaggatgtgtgcccagccaggcctccagcacccccagtgcagctcgtgattggaaactcaccatcggcaggcagtggttcggtttaagagatggcattagagggagcccagtctggatgtggacttggatgccctgtgggtatcagttctgctgacactttggcccgaaatagatccagtgctgagcaagcaatgtacaccggagcctcagtgagcccatctgcacagtggggagcatggagggatgggtttggcctgtgcttctgcttattcagtccttcagctcacggaagggatgctagtccgtgaaggtgacctc

SEQ ID NO: 306 - DNA, 537 bp, human
gactatgtctagagcctcttggggaaggggcacctctcttgaaccccaaatgctctctcttcttattacccaaacccatggctctatttcttcttcacatccattgtctcttcatgtctattccattcccttcggccaaacagacaggtggaaaaactgagacaggcagtttcagagatggacagagaactttattttggattgtggatgtggacttttttgtacataaataagaaaaaccaaaatactccaaagatgacttcccctgcctcctactccagtatgacagaggaggatgtaaggccttagccatgatctgcaggggtctgggagtcaggcccggcctattgcttgggtctctctctatttatatatctaagttcacagtgtttcttattccccctaagcttctagaggctcatggccctgtagttaggcctggctcattctgcacctttccagggaggtggaaggaccctgtgccctccttcccaatcttctttttcaggctcgccaaggcctaggacctatgttgta

SEQ ID NO: 307 - DNA, 539 bp, human
gctcctctactgtttaacaacatggctttcttattttgtttgaattgttaattcacagaatagcacaaactacaattaaaactaagcacaaagccattctaagtcattggggaaacggggtgaacttcaggtggatgaggagacagaatagagtgataggaagcgtctggcagatactccttttgccactgctgtgtgattagacaggcccagtgagccgcggggcacatgctggccgctcctccctcagaaaaaggcagtggcctaaatcctttttaaatgacttggctcgatgctgtgggggactggctgggctgctgcaggccgtgtgtctgtcagcccaaccttcacatctgtcacgttctccacacgggggagagacgcagtccgcccaggtccccgctttctttggaggcagcagctcccgcagggctgaagtctggcgtaagatgatggatttgattcgccctcctccctgtcatagagctgcagggtggattgttacagcttcgctggaaacctctggaggtcatctcggc

SEQ ID NO: 308 - DNA, 444 bp, human
atggcctggggctggcatttatctttcctttcagcaagcacctcaaatttgccatgctggctggtggaagagtttgtggtagcagaagagtgctctccatgctctaatttccgggctaaaactacccctgagtgtggtcccacaggatatgtagagaaaatcacatgcagctcatctaagagaaatgagttcaaaagctgccgctcagctttgatggaacaacgcttattttggaagttcgaaggggctgtcgtgtgtgtggccctgatcttcgcttgtcttgtcatcattcgtcagcgacaattggacagaaaggctctggaaaaggtccggaagcaaatcgagtccatatagctacattccacccttgtatcctgggtcttagagaccctatctcagacagtgaaagtgaaatggactgatttgcactcttggttctttgga

SEQ ID NO: 309 - DNA, 509 bp, human
ctgaagtgagaacccagctgtccgtcctcaggccggcctttcttccggcgacacccgtccatggctggctgggtccccttcgcagtgtttgtctgtcttgacatctaaaccccggcgtgtgcagtgcccatcttccaggactaccttattttccagaattaaacctgttttataattcaagttaatgcaaatgactgtcagttgccaaatatcttgatcctgtgagtgtagttgatgactgtttgttagtcagtagagtaaaatgctgtgtccacggggtgtcacagcctcaccataccctgttgaggtgtgaaatgccccgtcagaaattaaatacaaacttaaatgtgcctattggtgtctaaacttcatacaatgtaaggtcagattccttttaggaatactgggtgctgtcaccaggtttgatagttagacttaaaaacttgaaattcactttttggggggagggatatactgaaatagagagttgagacttgccagttggggga

SEQ ID NO: 310 - DNA, 457 bp, human
ctttcaccttaaagctctgccaagcagaggctcccgtgcaggaagagaagctgtcagcaagcacctcaaatttgccatgctggctggtggaagagtttgtggtagcagaagagtgctctccatgctctaatttccgggctaaaactacccctgagtgtggtcccacaggatatgtagagaaaatcacatgcagctcatctaagagaaatgagttcaaaagctgccgctcagctttgatggaacaacgcttattttggaagttcgaaggggctgtcgtgtgtgtggccctgatcttcgcttgtcttgtcatcattcgtcagcgacaattggacagaaaggctctggaaaaggtccggaagcaaatcgagtccatatagctacattccacccttgtatcctgggtcttagagaccctatctcagacagtgaaagtgaaatggactgatttgcactcttg

SEQ ID NO: 311 - DNA, 351 bp, human
aaaacagccaagcttttctgccaaaaagatgactgagaagactgttaaagcaaaaagctctgttcctgcctcagatgatgcctatccagaaatagaaaaattctttcccttcaatcctctagactttgagagttttgacctgcctgaagagcaccagattgcgcacctccccttgagtggagtgcctctcatgatccttgacgaggagagagagcttgaaaagctgtttcagctgggccccccttcacctgtgaagatgccctctccaccatgggaatccaatctgttgcagtctccttcaagcattctgtcgaccctggatgttgaattgccacctgtttgctgtgacat

SEQ ID NO: 312 - DNA, 504 bp, human
gatgccattatggctttccctgaagggtacaggacacaggtgggcgagcggggactgaagctgagcggcggggagaagcagcgcgtcgccattgcccgcaccatcctcaaggctccgggcatcattctgctggatgaggcaacgtcagcgctggatacatctaatgagagggccatccaggcttctctggccaaagtctgtgccaaccgcaccaccatcgtagtggcacacaggctctcaactgtggtcaatgctgaccagatcctcgtcatcaaggatggctgcatcgtggagaggggacgacacgaggctctgttgtcccgaggtggggtgtatgctgacatgtggcagctgcagcagggacaggaagaaacctctgaagacactaagcctcagaccatggaacggtgacaaaagtttggccacttccctctcaaagactaacccagaagggaataagatgtgtctcctttccctggcttatttcatcctggtcttggggta

SEQ ID NO: 313 - DNA, 372 bp, human
  misc_feature (43)..(43): n is a, c, g, or t
tccaaggactgagactgacctcctctggtgacactggcctagngcctgacactctcctaagaggttctctccaagcccccaaatagctccaggcgccctcggccgcccatcatggttaattctgtccaacaaacacacacgggtagattgctggcctgttgtaggtggtagggacacagatgaccgacctggtcactcctcctgccaacattcagtctggtatgtgaggcgtgcgtgaagcaagaactcctggagctacagggacagggagccatcattcctgcctgggaatcctggaagacttcctgcaggagtcagcgttcaatcttgaccttgaagatgggaaggatgttctttttacgtaccaattct

SEQ ID NO: 314 - DNA, 430 bp, human
ccgcacacggggtctgagactggcagtgatctccaactttgaccgacggctagagggcatcctggggggccttggcctgcgtgaacacttcgactttgtgctgacctccgaggctgctggctggcccaagccggacccccgcattttccaggaggccttgcggcttgctcatatggaaccagtagtggcagcccatgttggggataattacctctgcgattaccaggggcctcgggctgtgggcatgcacagcttcctggtggttggcccacaggcactggaccccgtggtcagggattctgtacctaaagaacacatcctcccctctctggcccatctcctgcctgcccttgactgcctagagggctcaactccagggctttgaggccagtgagggaagtggctgggccctaggccatggagaaaacct

SEQ ID NO: 315 - DNA, 534 bp, human
gatccatttttaacctctggtgctgtataaagggaatattaaactagatgtaaatcaaagtatatgtttggctcatttgagcattttggaatatttgagaatgtatgatacatgtaaaattaaaaaaactattagaactgtaccataattatgttgaaggtagaagtgatcttcaaagagatggccattaacttagcagtgggacctcacttttacaagcactgctctagatatacttgaagaatttaataggtacagaagtttattctggataataaataaataaggatcacactgtattaggggttatggcaacattattgaattttttatgtacataaagccatatgtttagggtggtttctatctgtcttgtttttcacttatataacactgtgaacttctaaagcaagaggataaaagaagcatgaatgaaaagaatgacatttcaaaaaaatggttcaatgaaaaactatagctaaaatatgtaaacctttctaggtaaaccgcttgccttcatcttgagtcggaata

SEQ ID NO: 316 - DNA, 463 bp, human
aaagcatcctgcagcgtgagcagctcctccacctggagctccgaagcatcttctcaggccaaagcggcattacccgtgaatctgtcttctccgccacagcatggtttgaggcgcagtctgttaatatagctgggccatgtcagtgactgttgtgtttgtggggtcaggtggggggcatggtatttgcaaaaaaaacaaattatggctaatttattattttgttgcagtggggttaactgtaaactcatgtaagagtctgtgatttcctcattggttgatctctctctctgtaatcctcattgcaaattttcaccaggacagcgttttttgattagaggggagctctggcacagtatgctttaatttagcaggaacttccagatgatttaaattctcgatgctgtgatgacacacatatgatctttcgtgtttctgagcgactctactttcattgtttgccagc

SEQ ID NO: 317 - DNA, 445 bp, human
agtgtattctagttgcaaagtatgcacacatatcttgaatggctttatttttattgtgtaaaactgttgaacacatgactgtgatgcacaaattctttacgtgtaaggagtctatgcattttacagtaacttattttatgatcgggtgatgagacagttatactttcaactgccattatttttattaagtgctttcattttctttacagttattataaaattgtatttattttatacagatgggttttcattttcctgatgctgtaatgtttacttcagcttgttgacctttctttgtgttatctgcatgttgtaacgtgtgataagaatgaatgtaaaggctgtggcaactgtaattaatttttgtaaagggctggtcacacgtggatctggtttatgaatgcatttgggatgattttggtaaccagatcaccttttcagaaat

SEQ ID NO: 318 - DNA, 554 bp, human
atgcctcctacgtgtattattgcgcaaatgagacggtgcatggtgtggagtttgactttatacccgatgtcaagggagcagtactggtttgtgacatgtcctcaaacttcctgtccaagccagtggatgtttccaagtttggtgtgatttttgctggtgcccagaagaatgttggctctgctggggtcaccgtggtgattgtccgtgatgacctgctggggtttgccctccgagagtgcccctcggtcctggaatacaaggtgcaggctggaaacagctccttgtacaacacgcctccatgtttcagcatctacgtcatgggcttggttctggagtggattaaaaacaatggaggtgccgcggccatggagaagcttagctccatcaaatctcaaacaatttatgagattattgataattctcaaggattctacgtgtctgtgggaggcatccgggcctctctgtataatgctgtcacaattgaagacgttcagaagctggccgccttcatgaaaaaatttttggagatgcatcagctatgaacacatcctaac

SEQ ID NO: 319 - DNA, 524 bp, human
cagggggactgcacaattccacctgcccaggacctgaccctggcgtgtgcttggccctcctcctcgcccacggcgcctcagatttcaggaccctcctcctcgcccacggcgcctcagacctcaggaccctgccgtctcacgcctttgtgaaccccaaatatctgagaccagtctcagtttattttgccaaggttaaggatgcacctgtgacagcctcaggaggtcctgacaacaggtgcccgaggtggctggggatacagtttgcctttatacatcttagggagacacaagatcagtatgtgtatggcgtacattggttcagtcagccttccactgaatacacgattgagtctggcccagtgaatccgcatttttatgtaaacagtaagggaacggggcaatcatataagcgtttgtctcaggggagccccagagggatgacttccagttccgtctgtcctttgtccacaaggaatttccctgggcgctaattatgagggaggcgtgtagcttcttatcattgt

SEQ ID NO: 320 - DNA, 490 bp, human
gtttcaccatacttcattcagaccagtgaaagagtagtgcatttaattggagtatctaaagccagtggcagtgtatgctcatacttggacagttagggaagggtttgccaagttttaagagaagatgtgatttattttgaaatttgtttctgttttgtttttaaatcaaactgtaaaacttaaaactgaaaaattttattggtaggatttatatctaagtttggttagccttagtttctcagacttgttgtctattatctgtaggtggaagaaatttaggaagcgaaatattacagtagtgcattggtgggtctcaatccttaacatatttgcacaattttatagcacaaactttaaattcaagctgctttggacaactgacaatatgattttaaatttgaagatgggatgtgtacatgttgggtatcctactactttgtgttttcatctcctaaaagtgttttttatttccttgtatctgtagtctttt

SEQ ID NO: 321 - DNA, 415 bp, human
tggtttcttccattggggcggagtcgggggtggagggagctggccacaacccactgctctgatgggtggtttgtccaaggatgctgaatgtaatgcctggtcaatgtggaagcccatgaggttgcccagggaagcctccaaaagctgggatgcttgagggtatccaagttgaaaaagacaaaatctgaccatcagccagtgacagtcctggcaaatgaaggtggggcggggcagtgaggggtgggagaaggtgaatgattcattattccaccccgaggtttgctggggtgaggggaagaatcgatgctgctttgggaactgaaggtttttctgttgggaaggccctcttggttttggagagaaagacaagttatgagtagctgctaccctggaacggtgggcagagagcctacta

SEQ ID NO: 322 - DNA, 391 bp, human
gaatttctgcaggcgccattgaagagtcaatcctttcccatggtaccgtggcagactgtgctgttgttggcaaggaagatcccttaaaaggtcatgtccccttagcactctgtgtattgagaaaagatataaatgcaacagaggagcaagttttggaagaaattgtgaaacacgttagacagaacattggccctgtggctgcttttcgaaatgcagtgtttgtcaaacagctacccaaaaccagatctggcaagatcccccgatcagctttatctgccattgtcaatggcaagccatacaagataacttctacaattgaagaccccagcatttttggccacgtagaagaaatgctgaagcaagcataatgagtttgtcttattcctatttt

SEQ ID NO: 323 - DNA, 371 bp, human
tattctgtcctgagaccacgggcaaagctcttccattttgagagagaagaaaaactgtttggaaccacaccaatgatatttttctttgtaatacttgaaatttatttttttattattttgatagcagatgtgctatttatttatttaatatatgtataaggagtcctaaacaatagaaagctgtagaagctgtagagataggcttcagttgttaattggtttggagcctcctatgtgtgacttatgactctctgtgttctgtgtatttgtctgaattaatgacctgggatataaagctatgctagctttcaaacaggagatgccttcagaaagctttgtatattttgcagttgccagaccaataaaatacc

SEQ ID NO: 324 - DNA, 318 bp, human
ttcgtgatctcataagcggactgacaagaaaaagcctggaatctgatttatcaacctggagagctgtactttttcagatctgtggattagtggcacttgtttagcatttaaaagggaaaaactccttgtctgtgtctcttctgacatgagaaacagtgatactgttacagcagaggtgaagtgtcaagctgtaggttcaccattcccaaaggcgtcggcagcccccgcaccatcgacgttctgtccccgattggtctgtggggtggccaatccagcacctgccaccagccctccactggggcctgtcctgcttctgcg

SEQ ID NO: 325 - DNA, 287 bp, human
ttttggggcatttccttacattgtcttgacaagattaaaatgtctgtgccaaaattttgtattttatttggagacttcttatcaaaagtaatgctgccaaaggaagtctaaggaattagtagtgttcccatcacttgtttggagtgtgctattctaaaagattttgatttcctggaatgacaattatattttaactttggtgggggaaagagttataggaccacagtcttcacttctgatacttgtaaattaatcttttattgcacttgttttgaccattaagctat

SEQ ID NO: 326 - DNA, 494 bp, human
ctccttgggctattcgatgccgtgtatgcatctgacagggcaacatttcatacaccatttagtcacctaggccaaagtccggaaggatgctcctcttacacttttccgaagataatgagcccagccaaggcaacagagatgcttatttttggaaagaagttaacagcgggagaggcatgtgctcaaggacttgttactgaagttttccctgatagcacttttcagaaagaagtctggaccaggctgaaggcatttgcaaagcttcccccaaatgccttgagaatttcaaaagaggtaatcaggaaaagagagagagaaaaactacacgctgttaatgctgaagaatgcaatgtccttcagggaagatggctatcagatgaatgcacaaatgctgtggtgaacttcttatccagaaaatcaaaactgtgatgaccactacagcagagtaaagcatgtccaaggaaggatgtgctgttacctctgatttccagtac

SEQ ID NO: 327 - DNA, 200 bp, human
tttttctcttctggcttcataaatgccttgctgtataaattgaaatattgatactgaactgtctttttaatgatgacctaactttattcaacccatcggaatttactttttccctgaaataagatcttttccactggtctactacctgaccataaacatgtctgcatttgaattctctaaaccctaaatctgtgtctatg

SEQ ID NO: 328 - DNA, 431 bp, human
aggagggctcactcaaagcctcctgcctatacgggcagctccccaagttccaggacggagacctcaccctgtaccagtccaataccatcctgcgtcacctgggccgcacccttgggctctatgggaaggaccagcaggaggcagccctggtggacatggtgaatgacggcgtggaggacctccgctgcaaatacatctccctcatctacaccaactatgaggcgggcaaggatgactatgtgaaggcactgcccgggcaactgaagccttttgagaccctgctgtcccagaaccagggaggcaagaccttcattgtgggagaccagatctccttcgctgactacaacctgctggacttgctgctgatccatgaggtcctagcccctggctgcctggatgcgttccccctgctctcagcatatgtggggcgc

SEQ ID NO: 329 - DNA, 555 bp, human
  misc_feature (31)..(31): n is a, c, g, or t
tagaggaacttgcctgtgagcgctggtcttngtgttnggtttngtgatgtaacgatctttgctggggttttttgcttngttttgagggaaatgtcttggagtaaattttaagttcctggagttaatttgttttacaggaatttngttttttaaaaaaataggatcattctgaactttggnaatgacccccttatatattttctgaaaatgaaaacagttacatgaaaaaaatttccaatgaagatgtcagcattttatgaaaaaccagaagttattagatgaaagcagcgagtgaatctttaaaacagacttgatcacgcacacacaataagtctttctctccgaaaccggaagtaaatctatatctgttagaaataatgtagccaaaagaatgtaaatttgaggatttttttgccaatagtttatagaaaatatatgaaccaaagtgatttgagtttgtaaaaatgtaaaatagtatgaacaaaatttgcactctaccagatttgaacatctagtgaggttcacattcatactaagttttcaacattgtgttct

SEQ ID NO: 330 - DNA, 496 bp, human
gaggttccccgtgtgtaggtagatctggtctttagaggcagatagataggtcagtgcaaatactctggtccatgggccatatgaaaaggctaagcttcactgtaaaataataactgggaattctggattgtgtatgggtgttggtgaacttggttttaattagtgaactgctgagagacagagctattctccatgtactggcaagacctgatttctgagcatttaatatggatgccgtgggagtacaaaagtggagtgtggcctgagtaatgcattatgggtggtttaccatttcttgaggtaaaagcatcacatgaacttgtaaaggaatttaaaaatcctactttcataataagttgcataggtttaataatttttaattatatggcttgagtttaaattgtaataggcgtaactaattttaactctataatgtgttcattctggaataatcctaaacatatgaattatgtttgcatgttcacttccaagagcc

SEQ ID NO: 331 - DNA, 464 bp, human
tccatcgctgtcatctcagctggatctccattctctcaggcttgctgccaaaagccttttgtgttttgttttgtatcattatgaagtcatgcgtttaatcacattcgagtgtttcagtgcttcgcagatgtccttgatgctcatattgttccctaatttgccagtgggaactcctaaatcaaattggcttctaatcaaagcttttaaaccctattggtaaagaatggaaggtggagaagctccctgaagtaagcaaagactttcctcttagtcgagccaagttaagaatgttcttatgttgcccagtgtgtttctgatctgatgcaagcaagaaacactgggcttctagaaccaggcaacttgggaactagactcccaagctggactatggctctactttcaggccacatggctaaagaaggtttcagaaagaagtggggacagagcagaactttcaccttcat

SEQ ID NO: 332 - DNA, 384 bp, human
accctcctcttacaaatgccaaaattcattcagctttgatgataaacactgaggcccaatggcctttatcatctagggagtatgaagaatgagcaagaggctaactcagcgtgagttaccctgggaaaggaaagaagaaatggttcatataggacacacatagataccttcaagggtgtttgtagagttgtttcttaagtagttggtttccttcacagaaagttcttaaactcagaatatacccatccatgcaccccaccagcaatacaaagaccccaaacaaaaaattactatattcttaccctacgcatggcttcctcctcttcttgacgcttttcataatgtgcaaagtcatcaaagattgaggtggtatgcttgaaagta

SEQ ID NO: 333 - DNA, 419 bp, human
ctcgtctgagccctgagtactacgacctggcaagagcccacctgcgtgatgaggagaaatcctgcccgtgcctggcccaggagggcccccagggtgacctgttgaccaaaacacaggagctgggccgtgactacaggacctgtctgacgatagtccaaaaactgaagaagatggtggataagcccacccagagaagtgtttccaatgctgcgacccgggtgtgtaggacggggaggtcacgatggcgcgacgtctgcagaaatttcatgaggaggtatcagtctagagttacccagggcctcgtggccggagaaactgcccagcagatctgtgaggacctcaggttgtgtataccttctacaggtcccctctgagccctctcaccttgtcctgtggaagaagcacaggctcctgtcctc

SEQ ID NO: 334 - DNA, 395 bp, human
ttcctctgtgttttctctattacagaaatggaatcctttgcagctacagctgacttcagggagcagattttaagagtaaaagaagatgagaatgttccatttctactggttggtaacaaatcagatttagaagataaaagacaggtttctgtagaagaggcaaaaaacagagctgagcagtggaatgttaactacgtggaaacatctgctaaaacacgagctaatgttgacaaggtattttttgatttaatgagagaaattcgagcgagaaagatggaagacagcaaagaaaagaatggaaaaaagaagaggaaaagtttagccaagagaatcagagaaagatgctgcattttataatcaaagcccaaactcctttcttatcttgaccatactaa

SEQ ID NO: 335 - DNA, 560 bp, human
gtgggagtggcctgaagagtcctctgaatgaaccttctggcctcccacagactcaaatgctcagaccagctcttccgaaaaccaggccttatctccaagaccagagatagtggggagacttcttggcttggtgaggaaaagcggacatcagctggtcaaacaaactctctgaacccctccctccatcgttttcttcactgtcctccaagccagcgggaatggcagctgccacgccgccctaaaagcacactcatcccctcacttgccgcgtcgccctcccaggctctcaacaggggagagtgtggtgtttcctgcaggccaggccagctgcctccgcgtgatcaaagccacactctgggctccagagtggggatgacatgcactcagctcttggctccactgggatgggaggagaggacaagggaaatgtcaggggcggggagggtgacagtggccgcccaaggccacgagcttgttctttgttctttgtcacagggactgaaaacctctcctcatgttctgctttcgattcgttaagagagcaacattttacccacaca

SEQ ID NO: 336 - DNA, 551 bp, human
gggacttataatttctgttgtttctaattaaagtccctgaagatcatataccaaagtgtttgagaacttcatccaaacctactttaaagcattatgtgcaattaagttgttatgacataattatattgcctaattgttgggtcttttttcttgagcttataatgtacctggaaaataaacctcttgagaaaaagaaaagttcatactgattattggaaaaggactatatatgtgagcaagattgtgttttagagaggaaacttgaaactccaagaaagcacttgatgtttttatatgcttgtagcaaattgatgttctaactgtagttttatagaaagtattaatgcttttatgtatttcaaaactttcatatgttaaatggaaattgttttaaatgtgtttgagtttatgtaagcatgtatacactgtgctaaaagtcacatgtttcagtttgtgtataatattaatatgcaatttttggtttaaatttttgtcttaaaatattagtggcttacattttaaaaaagaaaaatcaccagcatgaacttgca

SEQ ID NO: 337 - DNA, 540 bp, human
taagctaactctaaatttaatgctctacatcttatcagtcataattatatatactgtggaacagtatctgtagttactgcaaattactgtacagtttaggttataacagaaaactgacagagaagtaataaacctattgatttctctgcttataaatgaaagattgaaactatccaatgacatattatagtaaatgagtatctgtaacctcccactgcatcagaagcaggttaaatgaagtcttgtgaatttgtaatagatcagtaccatttattggtttggggaccatcttaattaaaaataaatgcccaaaatgtagaactttaaccaaagacttgtcccttttaaagcaaaatggggattgaagggacttataatttctgttgtttctaattaaagtccctgaagatcatataccaaagtgtttgagaacttcatccaaacctactttaaagcattatgtgcaattaagttgttatgacataattatattgcctaattgttgggtcttttttcttgagcttataatgtacctggaaa

SEQ ID NO: 338 - DNA, 559 bp, human
taccctcgcagcagtgtctctgaggactagcaaagtctggaggcagatgaatggtttctgaccctcaccagggctgtggaagggtgggggtgggtcattatagtattcaggatttacagtgcagtattcacgtgtaacttttaagttttcagtacagtgcttttatacctttaatgcaatgttgtattcatttgggtactattgtgtagtatttaggatgtatgcatgtttgtttatatgtaagcttggttggtgctttcgcttttgtgctacctttcttggatttttgtaccagagatgtgctaaactgatgaaatacattgagaaagtttccatcttattcttttatatgggactgatgatgtgtgttggggtagactgctcctgcagagtttggaagaagtcaccagcaaagccggcctaaccaagaaaagtcaaggcccttcatgaccttgctgggcacagaaaacaccctcgtggagtacactaatttgaactggactggtctcagtgtgagcacttggcacactttactaaacacatatacaaccccaccgtg

SEQ ID NO: 339 - DNA, 303 bp, human
ggaatgtcactggtgcccaggtgtacttgagctgtgaggtcatcggaatcccgacacctgtcctcatctggaacaaggtaaaaaggggtcactatggagttcaaaggacagaactcctgcctggtgaccgggacaacctggccattcagacccggggtggcccagaaaagcatgaagtaactggctgggtgctggtatctcctctaagtaaggaagatgctggagaatatgagtgccatgcatccaattcccaaggacaggcttcagcatcagcaaaaattacagtggttgatgccttacatg

SEQ ID NO: 340 - DNA, 455 bp, human
ggcccaaccgacagactatgggggctccggcaaccaggcgctgcgtggagtggctgctgggcctctacttcctcagccacatccccatcaccctgttcatggacctgcaggcggtgctgccgcgcgagctctacccagtcgagtttagaaacctgctgaagtggtatgctaaggagttcaaagacccactgctacaggagcccccagcctggtttaagtcctttctgttttgcgagcttgtgtttcagctgcctttctttcccattgcaacgtatgccttcctcaaaggaagctgcaagtggattcgaactcctgcaatcatctactctgttcacaccatgacaaccttaattccgatactctccacatttctgtttgaggatttctccaaagccagtggtttcaagggacaaagacctgagactttgcatgaacggttaacccttgtgtctgtc

SEQ ID NO: 341 - DNA, 412 bp, human
  misc_feature (365)..(365): n is a, c, g, or t
ataaaataggattctgcacagcagaaaaataaagccagagaccctcccccaacccctggtttgtgcaaagatactgggtatatgtaaacatgaagaggtaggaggtgcgagttcaggtcctggctccagatacagttaagctgctacaacaaccagggggacccagagggagcaccaggagggggaggaccccctcaagaggtgagaaggggtctgcggcctcgtctccagccgagggcgggaggcgcctcgcccctacacccatccgctccctccaacccaggccggggagggtacccacatggttccaggcaagtaataacaaaataacacggcatcccagttaatgctgcgtgcacggcggncgctgccggtcaaatctggaaggggaaggagctcaggtagtcgcgga

SEQ ID NO: 342 - DNA, 304 bp, human
  misc_feature (69)..(69): n is a, c, g, or t
tcctccactttaccttttgaaggagatccggactacaaaggaaaggtctttaacctctctgaatctcantgtgaaatgtgctcttccagttctatantaagatgtacctttgatgcatcaaccaatacagcaaaattgcctttcatagctaaaatcatgtaacttgagttccacagagcactccagcatatcatttatacaattactcatatatcctttatccccatattttaacatcttcatttatttggaaacactcaactacntgccaattcaaaagtcaaccacatccccatatgtaact

SEQ ID NO: 343 - DNA, 543 bp, human
cttgtacaaacgatgtgctttgaagatgttagtgtataacaattgatgtttgttttctgtttgattttaaacagagaaaaaataaaagggggtaatagctccttttttcttctttctttttttttttcatttcaaaattgctgccagtgttttcaatgatggacaacagagggatatgctgtagagtgttttattgcctagttgacaaagctgcttttgaatgctggtggttctattcctttgacactacgcacttttataatacatgttaatgctatatgacaaaatgctctgattcctagtgccaaaggttcaattcagtgtatataactgaacacactcatccatttgtgcttttgtttttttttatggtgcttaaagtaaagagcccatcctttgcaagtcatccatgttgttacttaggcattttatcttggctcaaattgttgaagaatggtggcttgtttcatggtttttgtatttgtgtctaatgcacgttttaacatgatagacgcaatgcattgtgtagctagttttctggaa

SEQ ID NO: 344 - DNA, 449 bp, human
ctcaaagactctcccgtggatgacggatgaggactctgggctgctggaataggacactcaagacttttgactgccattttgtttgttcagtggagactccctggccaacagaatccttcttgatagtttgcaggcaaaacaaatgtaatgttgcagatccgcaggcagaagctctgcccttctgtatcctatgtatgcagtgtgctttttcttgccagcttgggccattcttgcttagacagtcagcatttgtctcctcctttaactgagtcatcatcttagtccaactaatgcagtcgatacaatgcgtagatagaagaagccccacgggagccaggatgggactggtcgtgtttgtgcttttctccaagtcagcacccaaaggtcaatgcacagagaccccgggtgggtgagcgctggcttctcaaacggccgaagttgcctctttt

SEQ ID NO: 345 - DNA, 562 bp, human
atcatcagatcgactcttatacgagtaatatgcctgctatttgaagtgtaattgagaaggaaaattttagcgtgctcactgacctgcctgtagccccagtgacagctaggatgtgcattctccagccatcaagagactgagtcaagttgttccttaagtcagaacagcagactcagctctgacattctgattcgaatgacactgttcaggaatcggaatcctgtcgattagactggacagcttgtggcaagtgaatttgcctgtaacaagccagattttttaaaatttatattgtaaatattgtgtgtgtgtgtgtgtgtgtatatatatatatatatgtacagttatctaagttaatttaaagttgtttgtgcctttttatttttgtttttaatgctttgatatttcaatgttagcctcaatttctgaacaccataggtagaatgtaaagcttgtctgatcgttcaaagcatgaaatggatacttatatggaaattctgctcagatagaatgacagtccgtcaaaacagattgtttgcaaaggggaggcatcagtgtcttg

SEQ ID NO: 346 - DNA, 541 bp, human
  misc_feature (38)..(38): n is a, c, g, or t
gcaggtgaaatacttctaccccatgggtaaatgtattnacatattaccaagagaagaagcacattatctatgatcttttggcccagttcttatttagcatttttattccagcctacttggaaacatgtttttatttgcaatatatgcctgactgaattaagcttgcttgttttaaacaaccaaatcattggaacagaaaaggatttaaaaaacaanaatgcatgntctcagcgtgattaaaaaaaaantcagtggaaataaatgatcatagaaggtgcttttcaaaacaactgctattataattctcaaagtcctactctgccaaaagaagattaaaagtcatacattacattacaaggaaatgttcatgtgggaagagggttgctgaaaatcaacaacgcttgaagttaaaaagtgtgtctttgtagatttcattgtataatgtgtatttcttaggagatggctgacttgattgatctacgctaagtggagacatttcacatttttaaaaccaaatgttcaatctgtattactctttgcc

SEQ ID NO: 347 - DNA, 192 bp, human
aggtgaaggtgccgagctataaacctccagaatattattagtctgcatggttaaaagtagtcatggataactacattacctgttcttgcctaataagtttcttttaatccaatccactaacactttagttatattcactggttttacacagagaaatacaaaataaagatcacacatcaagactatctacaa

SEQ ID NO: 348 - DNA, 549 bp, human
tgttagtgcaagtcatcttccctacccatatttatcatcctcttgaggaaagaaatctagtattatttgttgaaaatggttagaataaaaacctatgactctataaggttttcaaacatctgaggcatgataaatttattatccataattataggagtcactctggatttcaaaaaatgtcaaaaaatgagcaacagagggaccttatttaaacataagtgctgtgacttcggtgaattttcaatttaaggtatgaaaataagtttttaggaggtttgtaaaagaagaatcaattttcagcagaaaacatgtcaactttaaaatataggtggaattaggagtatatttgaaagaatcttagcacaaacaggactgttgtactagatgttcttaggaaatatctcagaagtattttatttgaagtgaagaacttatttaagaattatttcagtatttacctgtattttattcttgaagttggccaacagagttgtgaatgtgtgtggaaggcctttgaatgtaaagctgcataagctgttaggttttgtt

SEQ ID NO: 349 - DNA, 438 bp, human
ggagctgccgggggtgcggcagagtgccctggactacttcatgttcgccgtgcgctgccgacaccagcgccgccagctggtgcacttcgcctgggagcacttccggccccgctgcaagttcgtctgggggccccaagacaagctgcggaggttcaagcccagctctctgccgcatccgctcgagggctccaggaaggtggaggaggaaggacctgcaggggacgagccagccgagagcccatcggagaccccaggccccagcccggcaggacctacaagggatgagccagccaaggcgggggaggcagaagcctgctgcctggctgtgtcttcccacccagctctcccctgcgcccctgtctttgttaatcgacccttctggagcggggggcggcgggcagggcttgcctttcttagtctgatgccaagcaaggcctt

SEQ ID NO: 350 - DNA, 470 bp, human
gcagggggccacggggaagataggccggagcccttaagccccaaagagagcaagaagaggaagctggagctgagccggcgggagcagccgcccacagagccaggccctcagagtgcctcagaggtggagaagatcgctctgaatttggaggggtgtgccctcagccagggcagcctcaggacggggacccaggaagtgggcggtcaggaccctggggaggcctcctgtccctgctgcaggggctggggcctccggagctgctgcgggctcccctcaggctctgcttcgtgacccgtgacccatgacccacagtgctggcctcctgtggggccactatagcagccaccagaagccgcgaggccctcagggaagcccaaggcctgcaggagcctcctggcctggctgtgtcttccccacccagctctcccctgcgcccctgtctttgtaaattgacccttctggagtggggg

SEQ ID NO: 351 - DNA, 386 bp, human
ctcattcttctaaaactactgcctttcaaagtgcacacacacgcgtccacatacactgcattcgttgctccagtataaattacatgcatgagcacctttctggcttttaagccaatataatgggctgcaaaatgaagacaccagagtgtatgcatacaaatctcactgtattaaagatgcaggttttctaattgtacccttcttgtctctctggcaatcttgcccttaatatccctggagttcctcatcagtgtcattttctgttatacacagttccacaattttgtctctagttgacttcaaatgtgtaactttattggtcttgccctattataattgtcatgactttcagattgtatctgaactcacagactgctgtcttacta

SEQ ID NO: 352 - DNA, 480 bp, human
  misc_feature (32)..(32): n is a, c, g, or t
ggagagagtcgttcgacctgctgctgagcagntaaaaatggagtggctgccttccacgctttcctgaagacagagttcagtgaggagaacctggagttctggctggcctgtgaggagttcaagaagatccgatcagctaccaagctggcctccagggcacaccagatctttgaggagttcatttgcagtgaggcccctaaagaggtcaacattgaccatgagacccgcgagctgacgaggatgaacctgcagactgccacagccacatgctttgatgcggctcaggggaagacacgtaccctgatggagaaggactcctacccacgcttcctgaagtcgcctgcttaccgggacctggctgcccaagcctcagccgcctctgccactctgtccagctgcagcctggacgagccctcacacacctgagtctccacggcagtgaggaagccagccgggaagagaggttgagtcacccatccc

SEQ ID NO: 353 - DNA, 513 bp, human
gtgggtctagggtcatctgaagggagtggtcccaagggtgcatgtggatgtcaacagaaggaccacaatggtgtgagaggccttgtgggcgtgcagagactggcagcagaaagtcccaagaggagaggactcagaggcttcttccttccaaggaggaggaggaggaggacattccacatctgggcttccagtggtggctcccggacctgccttgggggcagcaggagaggttgggagaggtcaccctgatgaagaacaatgccatgatctgctgctgccagaggacatctccgcccatccactcattttccacctgtacacattggctcattcattcgatggatatttcgggtgcatctactttatgccaggctctatgctaggagctggggatactgtggagaaaaagtcggatatggtctcgctctcatcaagatttcagtctagctgggagtgagcgttaaataagtcaataccaacacacacacgtgcacacacgtgatggcaagctgt

SEQ ID NO: 354 - DNA, 450 bp, human
gctggtatcagaacagcttccctcactgtgtacagaacgcaagaagggaataggtggtctgaacgtggtgtctcactctgaaaagcaggaatgtaagatgatgaaagagacaatgtaatactgttggtccaaaagcatttaaaatcaatagatctgggattatgtggccttaggtagctggttgtacatctttccctaaatcgatccatgttaccacatagtagttttagtttaggattcagtaacagtgaagtgtttactatgtgcaagggtattgaagttcttatgaccacagatcatcagtactgttgtctcatgtaatgctaaaactgaaatggtccgtgtttgcattgttaaaaatgatgtgtgaaatagaatgagtgctatggtgttgaaaactgcagtgtccgttatgagtgccaaaaatctgtcttgaaggcagctacactt

SEQ ID NO: 355 - DNA, 381 bp, human
tcccgtcgacgtagaacctcgaggtttctcaaagaaatttccactcaggtctgttttccgaggcggccccgggcgggtggattggatttgtctttggtgaacattgcaatagaaatccaattggatacgacaacttgcacgtattttaatagcgtcataactagaactgaattttgtctttatgatttttaaagaaaagttttgtaaatttctctactgtctcagtttacattttcggtatatttgtatttaaatgaagtgagactttgagggtgtatattttctgtgcagccactgttaagccatgtgttccaaggcattttagcggggagggggttatcaaaaaaaaaaaaaatgtgactcaagacttccagagcctca

SEQ ID NO: 356 - DNA, 504 bp, human
ggacgccatgaaggttttctgcaacatggagactggcgagacttgcgtctaccccaatccagcaaacgttcccaagaagaactggtggagcagcaagagcaaggagaagaaacacatctggtttggagaaaccatcaatggtggcttccatttcagctatggagatgacaatctggctcccaacactgccaacgtccagatgaccttcctacgcctgctgtccacggaaggctcccagaacatcacctaccactgcaagaacagcattgcctatctggacgaagcagctggcaacctcaagaaggccctgctcatccagggctccaatgacgtggagatccgggcagagggcaatagcaggttcacgtacactgccctgaaggatggctgcacgaaacataccggtaagtggggcaagactgttatcgagtaccggtcacagaagacctcacgcctccccatcattgacattgcacccatggacataggagggcccgagcagga

SEQ ID NO: 357 - DNA, 522 bp, human
tctatcccgtcaaattcaccttaaataacacccagctagatacaggcactaggtttgtgtaagatatgttgatacacacgaacaaagtttattttgactataatgtgtggactgactttcaacatttgcattttatctcacaaaggtgtatctattcaagtaacctttttttttttgtttgtttgtttcttttttgtttttttttttcttttggttgtttgtttcaattcatgtagctatttaaactgggataccttggactaagccagtctgtatcccaattcgctagcaagcctaagtttgtggggttttgtttttgtttttgttttaccttctaatttacaagaaagaggaaaagctcttctaactgaactttggtatgcggttgagctttgtaactatttgttctccatgaaaacaaaattatttatatttgacatatttttttctagtgtattaagttattttaaacaaaagatgttatctcatgacgtgttgtcagtacaaaatgtgtcgcctcca

SEQ ID NO: 358 - DNA, 459 bp, human
atactatttttcataccacgtgcatgtgaaagggactcatgtttgggtaggcctgtataagaatggcacccctgtaatgtacacctatgatgaatacaccaaaggctacctggatcaggcttcagggagtgccatcatcgatctcacagaaaatgaccaggtgtggctccagcttcccaatgccgagtcaaatggcctatactcctctgagtatgtccactcctctttctcaggattcctagtggctccaatgtgagtacaccccacagagctaatctaaatcttgtgctagaaaaagcattctctaactctaccccaccctacaaaatgcatatggaggtaggctgaaaagaatgtaatttttattttctgaaatacagatttgagctatcagaccaacaaaccttccccctgaaaagtgagcagcaacgtaaaaacgtatgtgaagcctctcttgaa

SEQ ID NO: 359 - DNA, 537 bp, human
ccaccgctggctgggaggagtcggagactgagacctacacagaggtggtgacagagtttgggaccgaggtggagcccgagtttgggaccaaggtggagcccgagtttgagacccagttggagcctgagttcgagacccagctggaacccgagtttgaggaagaggaggaggaggagaaagaggaggagatagccactggccaggcattccccttcacaacagtagagacctacacagtgaactttggggacttctgagatcagcgtcctaccaagaccccagcccaactcaagctacagcagcagcacttcccaagcctgctgaccacagtcacatcacccatcagcacatggaaggcccctggtatggacactgaaaggaagggctggtcctgcccctttgagggggtgcaaacatgactgggacctaagagccagaggctgtgtagaggctcctgctccacctgccagtctcgtaagagatggggttgctgcagtgttggagtaggggcagagggagggagccaaggtcactcca

SEQ ID NO: 360 - DNA, 549 bp, human
ttttgcttattttgttggagtattattgaagatgtgatcaatagattgtaatacacatatctaaaaatagttaacacagatcaagtgaacattacattgccatttttaattcattctggtctttgaaagaaatgtactactaaagagcactagttgtgaatttagggtgttaaactttttaccaagtacaaaaatcccaaattcactttattattttgcttcaggatccaagtgacaaagttatatatttataaaattgctataaatcgacaaaatctaatgttgtctttttaatgttagtgatccacctgcctcagcctcccaaagtgctgggattacaggcttgaaagtctaacttttttttacttatatatttgatacatataattcttttggctttgaaacttgcaactttgagaacaaaacagtcctttaaattttgcactgctcaattctgtttttcgtttgcattgtctttaatataataaaagttattacctttacatattatcatgtctatttttgatgactcatcaattttgtctatta

SEQ ID NO: 361 - DNA, 561 bp, human
gctacaacgatggactcttgcatagaaaaaaaaaatcttgttaacaatgaaaaaatgagcaaacaaaaaaatcgaaagacaaacgggagagaaaaagaggaaggcaacttatttcttaactgctatttggcagaagctgaaattggagaaccaaggagcaaaaacaaattttaaaattaaagtattttatacatttaaaaatatggaaaaacaacccagacgattctcgagagactggggggagttaccaacttaaatgtgtgttttaaaaaatgcgctaagaaggcaaagcagaaagaagaggtatacttatttaaaaaactaagatgaaaaaagtgcgcaggtgggaagttcacaggttttgaaactgacctttttctgcgaagttcacgttaatacgagaaatttgatgagagaggcgggcctccttttacgttgaatcagatgctttgagtttaaacccaccatgtatggaagagcaagaaaagagaaaatattaaaacgaggagagagaaaaataatggcaaaactgtctggactgctgacagtaaattccggttt

SEQ ID NO: 362 - DNA, 444 bp, human
  misc_feature (66)..(66): n is a, c, g, or t
agagaaagcgagacaggcccgtggacagtgattcccagacgggcagcggcaccatcctcacacctntgcantntgnnannngtctgaacagttgtttgtgtttttttttttttttttttnngacgaagaatgtttttatttttatttttttcatgcatgcattctcaagaggtcgtgccaatcagccactgaaaggaaaggcatcactatggactttctctattttaaaatggtaacaatcagaggaactataagaacacctttagaaataaaaatactgggatcaaactggcctgcaaaaccatagtcagttaattctttttttcatccttcctctgaggggaaaaacaaaaaaaaacttaaaatacaaaaaacaacattctatttatttattgaggacccatggtaaaatgcaaatagatccggtgtctaaatgcattcata

SEQ ID NO: 363 - DNA, 547 bp, human
gaacgtcgtatgagatcctacaatggaagaataaaatcacctcattcttcatttcagatctgaacattagcagtgatctagatttttttttttttaaacaaaattaagtgtgcttagagtcatccctctacatgggctgtggctgtcagcccataggtttgtcagtttcacatcaaaactgtgggtataaactgttgaaaccaatcacattaaaatatttagctgggcacagtggtgtgcatctgtagtcccagctacttgggaggctgaggcaggaggatcgcttaagcacaggagttggaatccagcctgagcaacagagcaaaaccccgtctctaaaatacaaataaaatatttgtgtagtttttgattaaaattgactacagcggtcagtataaaatacatgtcgcttttaaggaagtgctctttatgtatctaacagatggaagtttttgcattggtaagagcatttatatatgctttgtttcagggtttatggatttgtattcatatattgtcaaataggtttcatactctaattttactt

SEQ ID NO: 364 - DNA, 530 bp, human
gaaaatgctttcctgaacctggttcagtgcattcagaacaagcccctgtattttgctgatcggctgtatgactccatgaagggcaaggggacgcgagataaggtcctgatcagaatcatggtctcccgcagtgaagtggacatgttgaaaattaggtctgaattcaagagaaagtacggcaagtccctgtactattatatccagcaagacactaagggcgactaccagaaagcgctgctgtacctgtgtggtggagatgactgaagcccgacacggcctgagcgtccagaaatggtgctcaccatgcttccagctaacaggtctagaaaaccagcttgcgaataacagtccccgtggccatccctgtgagggtgacgttagcattacccccaacctcattttagttgcctaagcattgcctggccttcctgtctagtctctcctgtaagccaaagaaatgaacattccaaggagttggaagtgaagtctatgatgtgaaacactttgcctcctgtgtactgtgtcataaa

SEQ ID NO: 365 - DNA, 413 bp, human
aaattgtggagttagcctcctgtggagttagcctcctgtggtaaaggaattgaagaaaatataacaccttacaccctttttcatcttgacattaaaagttctggctaactttggaatccattagagaaaaatccttgtcaccagattcattacaattcaaatcgaagagttgtgaactgttatcccattgaaaagaccgagccttgtatgtatgttatggatacataaaatgcacgcaagccattatctctccatgggaagctaagttataaaaataggtgcttggtgtacaaaactttttatatcaaaaggctttgcacatttctatatgagtgggtttactggtaaattatgttattttttacaactaattttgtactctcagaatgtttgtcatatgcttcttgcaatgc

SEQ ID NO: 366 - DNA, 428 bp, human
gacaggtaacagtgtctgctgactccagtgcatccatgaactctggggttcttctggttcggccatcacggctctcctccagtgggactcccatgctagcaggggtctctgagtatgagcttcccgaagaccctcgctgggagctgcctcgggacagactggtcttaggcaaacccctgggagagggctgctttgggcaggtggtgttggcagaggctatcgggctggacaaggacaaacccaaccgtgtgaccaaagtggctgtgaagatgttgaagtcggacgcaacagagaaagacttgtcagacctgatctcagaaatggagatgatgaagatgatcgggaagcataagaatatcatcaacctgctgggggcctgcacgcaggatggtcccttgtatgtcatcgtggagtatgcctccaagggc

SEQ ID NO: 367 - DNA, 533 bp, human
  misc_feature (70)..(71): n is a, c, g, or t
gaaaatgctttcctgaacctggttcagtgcattcagaacaagcccctgtattttgctgatcggctgtatnngacnncangaagggcaaggggacgcgagataaggtcctgatcagaatcatggtctcccgcagtgaagtggacatgttgaaaattaggtctgaattcaagagaaagtacggcaagtccctgtactattatatccagcaagacactaagggcgactaccagaaagcgctgctgtacctgtgtggtggagatgactgaagcccgacacggcctgagcgtccagaaatggtgctcaccatgcttccagctaacaggtctagaaaaccagcttgcgaataacagtccccgtggccatccctgtgagggtgacgttagcattacccccaacctcattttagttgcctaagcattgcctggccttcctgtctagtctctcctgtaagccnaaagaaatgaacattccaaggagttggaagtgaagtctatgatgtgaaacactttgcctcctgtgtactgtgtcataaa

SEQ ID NO: 368 - DNA, 429 bp, human
tttattcctgggctaatcctgtccctccctaagtcacagcgaggtgtctgtcccagggctggagatgcttcccaaggaggagtctgttttgttgagagtgggcgtgggcttcttcacataagcctggggaaggaagaaaaaacggctttcattaccaaataatgtaaaacctcaaaagcaagggcttcaacagccttaaccaaatattattccccatagccagtggaaaatggatgtgacaaccccagtgcgcaggccagagtgagtgagcccagcacggcgctccgactggcttcctctctcaggtgctggattgtggggttagtggcatttccagctggattcctcctgttgtagttgccataaggaaatgagatgcagaatcagaaggatctatttctacagaatcatttcaccagttaagcacat

SEQ ID NO: 369 - DNA, 399 bp, human
tgctgaaaaccctccagtcagcgcttatcccttctgctctctcccctcacccagagaaatacatggagtttgaccttaatggaaatggcgatattgatatcatgtccctgaaacgaatgctggagaaacttggagtccccaagactcacctagagctaaagaaattaattggagaggtgtccagtggctccggggagacgttcagctaccctgactttctcaggatgatgctgggcaagagatctgccatcctaaaaatgatcctgatgtatgaggaaaaagcgagagaaaaggaaaagccaacaggccccccagccaagaaagctatctctgagttgccctgatttgaagggaaaagggatgatgggattgaaggggcttctaatgacccagatatgg

SEQ ID NO: 370 - DNA, 530 bp, human
ggcagattggagtttttcagagtatgtgtttcatctgctagtctttctctccttcatagcttttcttttcctggacttgctccttttgagttgcttttgcgtttctcatgcctaggcaagtgtaatagaaattatgtagctccttatgttggcaaaggagctctatatagtttcactttgtataaaagttaggaccagctgttgttacatgtaatattttagttcagaacttgacctgaaggaagggaagaaaagtatgtgatttttaccttttttaacaaatgtgaaaaagtcagttttagaaatttcgtggtagtaagttcggcatttgttacatgtatagagagaagactaataatctctatttataactaaatcattgagatagaaaaagattcccattgactgtagacttcttcccattttgtcttcccttctgcctgtttccccttcaggcttggctctaggaaccaaagtgatttgttgttgttccaacctgggctttgtgactttggttagtgccactacct

SEQ ID NO: 371 - DNA, 480 bp, human
ccccgcatgcttcaggagaacacaaggatggacatggaaaatgaagaaagcatgatgtcctatgagggagacggtggggaggcttcccatggtttggaggatagcaacatcagttatgggagctatgaggagcctgatcccaagtcgaacacccaagacacaagcttcagcagcatcggtgggtatgaggtatcagaggaggaagaagatgaggaggaggaagagcagcgctctgggccgagcgtactaagccaggtccacctgtcagaggacgaggaggacagtgaggatttccactccattgctggggacagtgacttggactctgatgaatgaggcttcctttgggcctccttggtcagtatctgatcctgaaatcatgaaattaactaacaccttagcctttttaaaagtagtaagtaaatgataataaatcacctctcctaatcttcctggggcaatgtcaccctttgatttaaa

SEQ ID NO: 372 - DNA, 458 bp, human
agaaaatttgccaatctttcctactttctatttttatgatgacaatcaaagccggcctgagaaacactatttgtgactttttaaacgattagtgatgtccttaaaatgtggtctgccaatctgtacaaaatggtcctatttttgtgaagagggacataagataaaatgatgttatacatcaatatgtatatatgtatttctatatagacttggagaatactgccaaaacatttatgacaagctgtatcactgccttcgtttatatttttttaactgtgataatccccacaggcacattaactgttgcacttttgaatgtccaaaatttatattttagaaataataaaaagaaagatacttacatgttcccaaaacaatggtgtggtgaatgtgtgagaaaaactaacttgatagggtctaccaatacaaaatgtattacgaatgcccctgttcatgtt

SEQ ID NO: 373 - DNA, 467 bp, human
  misc_feature (153)..(153): n is a, c, g, or t
ggaaaggattcttggacctctaccaaaacatatgatacagaaaaccaggaaacgtaaatattttcaccacgatcgattagactgggatgaacacagttctgccggcagatatgtttcaagacgctgtaaacctctgaaggaatttatgctttnctcaagatgtngaacatgagcgtctctttgacntcattcagaaaatgttggagtatgatccagccaaaagaattactctcagagaagccttaaagcatcctttctttgaccttctgaagaaaagtatatagatctgtaattggacagctctctcgaagagatcttacagactgtatcagtctaatttttaaattttaagttattttgtacagctttgtaaattcttaacatttttatattgccatgtttattttgtttgggtaatttggttcattaagtacatagctaaggtaatgaacatctttttcagtaat

SEQ ID NO: 374 - DNA, 509 bp, human
ctcaccatctcggagcggtggcagcaggaggtggcagagactgtcttcgacaccatcaacgctgagacagaccggctggaggctcgcaagaaagccaagaacaagcagctgggccatgaggaagactacgccctgggcaaggactgcatcatgcatggctacatgtccaagatgggcaaccccttcctgacccagtggcagcggcggtacttctacctgttccccaaccgcctcgagtggcggggcgagggcgaggccccgcagagcctgctgaccatggaggagatccagtcggtggaggagacgcagatcaaggagcgcaagtgcctgctcctcaagatccgcggtgggaaacagttcattttgcagtgcgatagcgaccctgagctggtgcagtggaagaaggagctgcgcgacgcctaccgcgaggcccagcagctggtgcagcgggtgcccaagatgaagaacaagccgcgctcgcccgtggtggagctgagcaaggtgccgct

SEQ ID NO: 375 - DNA, 465 bp, human
  misc_feature (110)..(110): n is a, c, g, or t
acacacacatgcaattttgcttaacaaaagtattttataatacagtttcatacagaattaccttaaaagggagtcttatgttttcaactacagatagttgtaagggatcntacagaagatattgatgatagttgaaatattcttagaaggggtgtgtatgtctagctgtgtctaccatgtgtatgtattcttgacaagcagtataaaatacctgtgatttttctttacattagggataatgcataaggaattaatcttcatatatattatcatccctaatgtagcagggggaagtatttaattgcccatgatatgtattttacttatactatgccagagaggaaactataaagtaattacacatgtaatcttgggtttttcacatatgtaggtattcattttgagtaggttgaagaagaaaaaaaatatttaaatgaattgaattcctgatgggatagtatcaat

SEQ ID NO: 376 - DNA, 555 bp, human
gtattttgtgtgaccaactgtgttgtattaacaaaagttccctgaaacacgaaacttgttattgtgaatgattcatgttatatttaatgcattaaacctgtctccactgtgcctttgcaaatcagtgtttttcttactggagcttcattttggtaagagacagaatgtatctgtgaagtagttctgtttggtgtgtcccattggtgttgtcattgtaaacaaactcttgaagagtcgattatttccagtgttctatgaacaactccaaaacccatgtgggaaaaaaatgaatgaggagggtagggaataaaatcctaagacacaaatgcatgaacaagttttaatgtatagttttgaatcctttgcctgcctggtgtgcctcagtatatttaaactcaagacaatgcacctagctgtgcaagacctagtgctcttaagcctaaatgccttagaaatgtaaactgccatatataacagatacatttccctctttcttataatactctgttgtactatggaaaatcagctgctcagcaacctttcacctttgtgtat

SEQ ID NO: 377 - DNA, 531 bp, human
agtgggaacgtcttacggtagctgatgcattggaaccagtgcagtttgaagatgggcagaagattgtggtgcagggagaaccaggggatgagttcttcattattttagaggggtcagctgctgtgctacaacgtcggtcagaaaatgaagagtttgttgaagtgggaagattggggccttctgattattttggtgaaattgcactactgatgaatcgtcctcgtgctgccacagttgttgctcgtggccccttgaagtgcgttaagctggaccgacctagatttgaacgtgttcttggcccatgctcagacatcctcaaacgaaacatccagcagtacaacagttttgtgtcactgtctgtctgaaatctgcctcctgtgcctcccttttctcctctccccaatccatgcttcactcatgcaaactgctttattttccctacttgcagcgccaagtggccactggcatcgcagcttcctgtctgtttatatattaaagttgcttttattgcaccattttcaatttggagca

SEQ ID NO: 378 - DNA, 444 bp, human
ctatgagatggccacaggccgtcccctctttccgggctccacggtggaggaacagctacacttcatcttccgtatcttaggaaccccaactgaggagacgtggccaggcatcctgtccaacgaggagttcaagacatacaactaccccaagtaccgagccgaggcccttttgagccacgcaccccgacttgatagcgacggggccgacctcctcaccaagctgttgcagtttgagggtcgaaatcggatctccgcagaggatgccatgaaacatccattcttcctcagtctgggggagcggatccacaaacttcctgacactacttccatatttgcactaaaggagattcagctacaaaaggaggccagccttcggtcttcgtcgatgcctgactcaggcaggccagctttccgcgtggtggacaccgagttctaagccacaga

SEQ ID NO: 379 - DNA, 512 bp, human
  misc_feature (166)..(166): n is a, c, g, or t
ggttagattctcagaatgcccagggatatgaatggatattcattgcatggtctccagatcattctcatgttcgtcaaaaaatgttgtatgcagcaacaagagcaactctgaagaaggaatttggaggtggccacattaaagatgaagtatttggaacagtaaaggnaagatgtatcattacatggatataaaaaatacttgctgtcacaatcttcccctgccccactgactgcagctgaggaagaactacgacagattaaaatcaatgaggtacagactgacgtgggntgtggacactaagcatcaaacactacaaggagtagcatttcccnatttcntcgagnannctttcaggctttggaaaaattgaataatagacagctcaactatgtgcagttggaaatagatataaaaaatgaaattataattttggccaacacaacaaatacagaactgaaagatttgccaaagaggattcccaaggattcagctcgttaccatttctttctgta

SEQ ID NO: 380 - DNA, 382 bp, human; identical to SEQ ID NO: 119
  misc_feature (57)..(57): n is a, c, g, or t

SEQ ID NO: 381 - DNA, 361 bp, human
gtaggaccaagtgatgacacccctcgaaggggcccagagtccctgcccgtgggacatcatgacatcatcactgatgtcgccacattccagaccacacagggcttcatcgtaactgcttctagagatgggattgtgaaggtgtggaaataaaacctactgatttgtataaattttaatagttataaatataatactataactcgagaaaaggcatttctagagaacagattcatttgcttaattttcaaaattatgtctccatattactgtttcatgactgactgactaaatgacacccaaaatggttaagatgtacttgactagtttacttatgcatctctttgcaagaatcagccagcca

SEQ ID NO: 382 - DNA, 563 bp, human
  misc_feature (73)..(73): n is a, c, g, or t
ggaaaatcactccctagaggtggagtcatgtttgagtaatgtttggttaactgaaagaaggctagtatggctncatggtagtggtgaggaagtaacaaaaattagagcggggtagcaggtaagggtcagaccagcagggacttgaagaccaaggtaagacattttttactttattcaaaaggnaaaggaagacttttaagtagggaagaattttctttcaatttacattcttaaaacaatcctgcgggctgccaagtggagaatggactagaggcaggaagagtggaagccagcatccagataggagactcctagagtggtcccaatggaaaccaatgagggcttgggatgcagcaggggcagaaggagagaagatggtagattctccagatatattttcagagttaaaagcagtaagacttgatgatgaattagtcatggaaaagtaagggagagagttaaagatgactcccagacttcctgctagggccttagtatgataccatttactcccatttaccaccgtttaagaaggggctgaggcaggacattccacgcatgtc

SEQ ID NO: 383 - DNA, 216 bp, human
cacaatgcgcgttgactacgagcagatcaagataaaaaagattgaagatgcatccaaccctctgctgctgaagaggcggaagaaagctcgggccctggaggctgcggctctggcccactgagccaccgcgccctcctgcccacgggaggacaagcaataactctctacaggaatatattttttaaacgaagagacagaactgtccacatctgcctc

SEQ ID NO: 384 - DNA, 485 bp, human
tggctctgtggcgagccatttgtcccaagtgccaaagaagcagaccattggggctcccagccaggcccttgtcggccccaccagtgcctctccctgctgctcctaggacccgtctccagctgctgagatcctggactgagggggcctggatgccccctgtggatgctgctgcccctgcacagcaggctgccagtgcctgggtggatgggccaccgccttgcccagcctggatgccatccaagttgtatatttttttaatctctcgactgaatggactttgcacactttggcccagggtggccacacctctatcccggctttggtgcggggtacacaagaggggatgagttgtgtgaataccccaagactcccatgagggagatgccatgagccgcccaaggccttcccctggcactggcaaacagggcctctgcggagcacactggctcacccagtcctgcccgccaccgttatcggtgtcattc

SEQ ID NO: 385 - DNA, 476 bp, human
aaatggcccttttgaatgttgaagataggaaagaaagccatatattcaaatatgtaattttcctcatttagttataatgacatctttaccaattgggcttcataaatgtgtttctttcttaatagtatcctagttccagcatatattcaacatgaattttatcattctcctcctaatggaatgtcttccttatagaaatgttcacgacaaattcatttgtgttttttacatgtcaataatgtatgctaaattaaaatgttttccagttattctgatagatgtcattatggcatccttaatttttcttctccttctgtatatagggtaagggactgttctgaagaacctttccatttagtgatcaagatatggaagctgatttctgaaaatgctcagtgtgtactctaattatttatggtaccatttgaattgtaacttgcattttagcagtgcatgtttctaattgacttactggg

SEQ ID NO: 386 - DNA, 574 bp, human
  misc_feature (142)..(149): n is a, c, g, or t
ttctctaagttaacagaagaggaactattggacagagaatttgaccttctaagatcaagtaagggacacttgaaggcttattttgcttcacagtaaaataacagctctattattattcagcaaggccaaagacttttgagannnnnnnngnaaantnnttttnnnnnnttnnnnnnnnnnnnnnnnncatcttcttatacatatactatcaaattatgttgtgtgncattagaaatcagttgcttgatagtagctattaaacccaatattgctgatagtatgctaatatcctaaaacttaaatattgcatatctatgaatgttaaattcagaatatctctaaaacatggaaaattgatgtttcaataaaaagggagacattttattattttgccttactaatgattttgcagctctgtttttctgcacactcaatagaaaatattgtgggtctgagatgccctttgaaaatgcctgaaagaaaacatgggctactacattatgttaatgttttgtaatgtccttttaatgagtggtgacataaaagggctgctttgttgtcctgatatggcaaa

SEQ ID NO: 387 - DNA, 559 bp, human
tggggatgccagtaactttgacgactacgaggaggaagagctccggatctccatcaatgagaagtgtgccaaggagttttctgagttttaggggtgtgcttgtgcccctgtggttttctttcctttttgtttttggtggtttgggggatgggagggttggattgaacagccagagggccccagagttccttgtatctaatttcatcctcacccacctccagggttgggggagcaggaagcccagatatttggaggaacagaaacaccagctgctccctcacccccccccatgccttcctggtccctctgtgcttctctctttctcctccacaggtcccccttgccccagcccccttctgcctgttttaaacgagtttctcagctctattcaggccaggtcttgctgttgtatcaagggacacggtgtggaaagaggggctcaaacttaactccagccctgaacaggcaccacttactaagagaggatgaatgaaaagcacacctaccctttggcgtaatcctgcctgggaaggagagaggtttagtgccatgttcagtg

SEQ ID NO: 388 - DNA, 373 bp, human
ccaggaagctgatccaccttgagatcaagccggccatccggaaccagatcatccgcgagctgcaggtcctgcacgaatgcaactcgccgtacatcgtgggcttctacggggccttctacagtgacggggagatcagcatttgcatggaacacatggacggcggctccctggaccaggtgctgaaagaggccaagaggattcccgaggagatcctggggaaagtcagcatcgcggttctccggggcttggcgtacctccgagagaagcaccagatcatgcaccgagatgtgaagccctccaacatcctcgtgaactctagaggggagatcaagctgtgtgacttcggggtgagcggccagctcatagactccat

SEQ ID NO: 389 - DNA, 476 bp, human
tcagatgtgtggtcctttggcgtgctcctctgggagctgatgacaagaggagccccaccttatcctgacgtaaacacctttgatataactgtttacttgttgcaagggagaagactcctacaacccgaatactgcccagaccccttatatgaagtaatgctaaaatgctggcaccctaaagccgaaatgcgcccatccttttctgaactggtgtcccggatatcagcgatcttctctactttcattggggagcactatgtccatgtgaacgctacttatgtgaacgtaaaatgtgtcgctccgtatccttctctgttgccatcagaagataacgctgatgatgaggtggacacacgaccagcctccttctgggagacatcatagtgctagcactatgtcaaagcaacagtccacactttgtccaatggttttttcactgcctgacctttaaaaggccatcgatattctttgctctt

SEQ ID NO: 390 - DNA, 467 bp, human
gattctgttaccttgactgggggtggaacactacattctttttagagtctgattttatggattggaatattgggatatctttctttcctttatttatttgaaataattagtgtagtgattacaaaacagtcataaatttttaaaggccttttttctctcttttttttttttttagaatagtatttttttttaagtcctttatgtaacatccagataatgtgatactgtctctttgaagcaccctgtaaaccttttagagatttgaagttgggtcttgactcttaatgcatgtggacagtcgcgagcgtttatgctgtcggtgtgtctgtgttggacaaaacaatctgtaatctacagcaaagtacattctacattccgttcatggggcacacccaggggaataagaataaaatgctattatgactaagttgtaaacctatgcacatcccttgcattttgggcaactt

SEQ ID NO: 391 - DNA, 464 bp, human
tgcctccctgctgagaatattctccctttagcagccaaagattcgctggaacggaggctgccctctgctgcctgttggggtcggaagacaaggggcttctgaaatgggagttcctgagatacaacaaaatgtgtgccttcaaagaaactgacagctttgtatttggtgaaatggttttaattatactccatgtgtattttgcccactttttttgggaattcaagggaaagtgtttcttgggtttggaatgttcagaggaagcagtattgtacagaacacggtattgttatttttgttaagaatcatgtacagagcttaaatgtaatttatatgtttttaatatgccattttcattgaagtattttggtcttaagatgactttagtaatttaactgtttatgttacccacgttgggatccagttggtcttggtttgcttctctctgtaccacgtgcacatgaggt

SEQ ID NO: 392 - DNA, 547 bp, human
tcttccccagggttctgtattgcagctaagctcaaatgtatatttaacttctagttgctcttgctttggtcttcttccaatgatgcttactacagaaagcaaatcagacacaattagagaagccttttccataaagtgtaattttaatggctgcaaaaccggcaacctgtaactgcccttttaaatggcatgacaaggtgtgcagtggccccatccagcatgtgtgtgtctctatcttgcatctacctgctccttggcctagtcagatggatgtagatacagatccgcatgtgtctgtattcatacagcactacttacttagagatgctactctcagtgtcctcagggctctaccaagacataatgcactggggtaccacatggtccatttcatgtgatctattactctgacataaacccatctgtaatatattgccagtatataagctgtttagtttgttaattgattaaactgtatgtcttataagaaaacatgtaaagggggaatatattgggggagtgagctctctcagacccttgaagatgt

SEQ ID NO: 393 - DNA, 476 bp, human
cgcaccccgacttgatagcgacggggccgacctcctcaccaagctgttgcagtttgagggtcgaaatcggatctccgcagaggatgccatgaaacatccattcttcctcagtctgggggagcggatccacaaacttcctgacactacttccatatttgcactaaaggagattcagctacaaaaggaggccagccttcggtcttcgtcgatgcctgactcaggcaggccagctttccgcgtggtggacaccgagttctaagccacagaccgaggccccagcaggcagcggctggagggatgccacacccctcacagggcagcccccaactacatcttccctgcttactctctgcctacctgcctgagccatgttcacctgcccacttgtcccctgctgcctgcccaaacaccccaccattggcctgtcaacccacccattggcctgtctgctgggtgctaacaaagctctcatcact

SEQ ID NO: 394 - DNA, 529 bp, human
gcagccaaggtctgtgttcagcacttggtctctgttgttacgtaaaataataagcatttaaaatagtttacagatatttttgaccagttccttttagagattctttcagagaagaaaccagatctgacctgtttattgttggcgcttgttgaaaacgagctttctttcccatgatagtgcttcgtttttgaagtgttgaagctgtgctccccttaaatcgtggcaggagagattaaggtaattacaacactcagttctatgtcttacaagcactttgtcttgtctctgcaagaaaattcgattccagtcatttcccataaaatacagacattttaccaacataatatgctttgattgatgcagcattatgctttgggcagtattacaaaatagctggcgagtgctttctgtatttaaatattgtaaaaagaaaataagttataactgttataaagcagaacttttgttgcattttttaaactgttgaagtcactgtgtatgtttgtttggtcaatgtttccgcagtatt

SEQ ID NO: 395 - DNA, 536 bp, human
  misc_feature (26)..(26): n is a, c, g, or t
aaaatgctattagtccgtcgtgcttnatttgtttttgtccttgaataagcatgttatgtatatngtctcgtgtttttatttttacaccatattgtattacacttttagtattcaccagcataancactgtctgcctaaaatatgcaactctttgcattacaatatgaagtaaagttctatgaagtatgcattttgtgtaactaatgtaaaaacacaaattttataaaattgtacagttttttaaaaactactcacaactagcagatggcttaaatgtagcaatctctgcgttaattaaatgcctttaagagatataattaacgtgcagttttaatatctactaaattaagaatgacttcattatgatcatgatttgccacaatgtccttaactctaatgcctggactggccatgttctagtctgttgcgctgttacaatctgtattggtgctagtcagaaaattcctagctcacatagcccaaaagggtgcgagggagaggtggattaccagtattgttcaataatccatggttca

SEQ ID NO: 396 - DNA, 501 bp, human
acagctcttttaatacaatttccttggtatccaacttaaatcccagaattttgttccaccatggttagttatttgccatatgaatgtattcttgtttctaccaatattctaggcatgagaataccttaatactgagttggagattttgtatctgtttctcctcctttacgcccattttcctacccacagcagcaaatgacaacgtgtctgtccaggtctgtccccctgctcatcccaggatgccactcacatttttttcttctttgttacccttgacccacgctgtacaagtaacatccaagagcccattctacaagtgggtggttttggtctttttataactttttctcaaagtcactgatgtttgttcctgttaaatgtatagcattgtaatgagagcccatcaaatcctgagtgtcagtttgttgtccctattgtagatgaaatagtgatgtagcaaaaacctagtaaattctgaatgcttttccacgtagacttatc

SEQ ID NO: 397 - DNA, 396 bp, human
ttcctgaagctcttcggctgacagcccgctcggctcgccctctcccccggaggccgcgccctcccggaaaagccgggtcctccagaccccgaggccttgctctcagagcgggaggccccacccactggagagccccgcccctaaggtactatgagttctcaggggtcaagttcagaaacggcccagccagacctaaacccacacgcccacaaagtggattgcacacagacaagaactcccgtgcgggcctccactctattcccacccttgagggagcccccttactgggggagggtccttgcaattccagcgaatcggaggccaggccaggacgtccttgctccctgcaccctcactgttctgtgcactttttctacctacataaacacacgcatt

SEQ ID NO: 398 - DNA, 241 bp, human
aagagtcagctgatgccagaaggaaagccgaaatgctacaaaatgaagcaaaaactcttttagctcaagcaaatagcaagctgcaactgctcaaagatttagaaagaaaatatgaagacaatcaaagatacttagaagataaagctcaagaattagcaagactggaaggagaagtccgttcactcctaaaggatataagccagaaagttgctgtgtatagcacatgcttgtaacagaggag

SEQ ID NO: 399 - DNA, 487 bp, human
aaagttttcacgtcatcgtttcctggctagttcatttcattaagtggctacatcctaacatatgcattggtcaaggttgcagcaagaggactgaagattgactgccaagctagtttgggtgaagttcactccagcaagtctcaggccacaatggggtggtttggtttggtttccttttaactttctttttgttatttgcttttctcctccacctgtgtggtatattttttaagcagaattttattttttaaaataaaaggttctttacaagatgataccttaattacactcccgcaacacagccattattttattgtctagctccagttatctgtattttatgtaatgtaattgacaggatggctgctgcagaatgctggttgacacagggattattatactgctatttttccctgaattcttttccttggaattccaactgtggaccttttatatgtgccttcactttagctgtttgccttactct

SEQ ID NO: 400 - DNA, 435 bp, human
  misc_feature (261)..(261): n is a, c, g, or t
gtttgagttttgtagggccatcacctggaaagtcaatgtgactagacacaaagtagcccagaggctacttttcttcctacagcttattatagttgtaggttctatgacctcacttcatgggttccaggcaattccgctgaaaggtttgtctcctgaaattttttaagtttgttttcctgacacatgtaatcagatgtgtagcaaccgagggaaacgaagcctaacattctccattgtggaaatacacacaggaggttacanttcacagcgtggatttttccagcttacacatgtgggatgacatcacagaaaccacaaaagcagcaaattaaactgtaggagagtcaatactcctgacgagtctcnggggggggcatttttatgccttcttaactttatgagaattctcaggctgaactataggccattgttccc

SEQ ID NO: 401 - DNA, 546 bp, human
caggaaatagtcactcatcccactccacataaggggtttagtaagagaagtctgtctgtctgatgatggatagggggcaaatctttttcccctttctgttaatagtcatcacatttctatgccaaacaggaacgatccataactttagtcttaatgtacacattgcattttgataaaattaattttgttgtttcctttgaggttgatcgttgtgttgttttgctgcactttttacttttttgcgtgtggagctgtattcccgagacaacgaagcgttgggatacttcattaaatgtagcgactgtcaacagcgtgcaggttttctgtttctgtgttgtggggtcaaccgtacaatggtgtgggaatgacgatgatgtgaatatttagaatgtaccatattttttgtaaattatttatgtttttctaaacaaatttatcgtataggttgatgaaacgtcatgtgttttgccaaagactgtaaatatttatttatgtgttcacatggtcaaaatttcaccactgaaaccctgcacttagctagaacct

SEQ ID NO: 402 - DNA, 384 bp, human
  misc_feature (246)..(246): n is a, c, g, or t
gaagaaacacagcctcatgggacagaaatttgagggaggggaacaaagaatactttggggggaaaagagttttaaaaaagaaattgaaaattgccttgcagatatttaggtacaatggagttttcttttcccaaacgggaagaacacagcacacccggcttggacccactgcaagctgcatcgtgcaacctctttggtgccagtgtgggcaagggctcagcctctctgcccacagagtgcccccangtggaacattctggagctggccatcccaaattcaatcagtccatagagacgaacagaatgagaccttccggcccaagcgtggcgctgcgggcactttggtagactgtgccancacggcgtgtgttgtgaaacgtgaaa

SEQ ID NO: 403 - DNA, 393 bp, human
gaaattctttcagctatcagtaggctaatgtcaaaattgtttaaaaattcttgaaagaattttcctgagacaaattttaacttcttgtctatagttgtcagtattattctactatactgtacatgaaagtagcagtgtgaagtacaataattcatattcttcatatccttcttacacgactaagttgaattagtaaagttagattaaataaaacttaaatctcactctaggagttcagtggagaggttagagccagccacacttgaacctaataccctgcccttgacatctggaaacctctacatatttatataacgtgatacatttggataaacaacattgagattatgatgaaaacctacatattccatgtttggaagacccttggaagag

SEQ ID NO: 404 - DNA, 193 bp, human
  misc_feature (88)..(88): n is a, c, g, or t
gaggactgcagcctagcaggtcaggataagtactccagcacaagaaagtctcatggggaccttcttcctgcccctcttaaaccacggncacccccctgccagccacgcccggcctccgatggcgtctttctctccaactcattccagccaccctcagccaaagccaacagtactgctcacaaaaaaggccagc

SEQ ID NO: 405 - DNA, 469 bp, human
gagacggcaggaggcctgagcgatccgtactcagaacgtccaggagagacgcatggcccgaagtcaaagtgctggaattttccaaaacagcctgttctctcctctctcctccccagagcaccccctgccatcaggggggttgaaatccctctcccccaggagccctgctgctttgcttggtggtagggcaggagagcaaacaaacagtcatggtctaaaacccacatagcactttgctcttagttacatgtaaaattttagatttctaaaacaggtgggcaatcattttgaatactgttctgtgaccctgactgctagttctgaggacactggtggctgtgctatgtgtggccatcctccatgtcccgtccctgtagctgctctgtttagacagcggacagacgctcacgcccaggggatgtcctcacgctgtcgccgcgcggtttcccttcgcagatgtgtatactca

SEQ ID NO: 406 - DNA, 543 bp, human
catgactgcatgacggagccgagcatgtgtctttacacctcgctgttgtcacatctcagggaactgaccctcaggcacaccttgcagaaggaaggccctgccctgcccaacctctgtggtcacccatgcatcattccactggaacgtttcactgcaaacacaccttggagaagtggcatcagtcaacagagaggggcagggaaggagacaccaagctcacccttcgtcatggaccgaggttcccactctggcaaagcccctcacactgcaagggattgtagataacactgacttgtttgttttaaccaataactagcttcttataatgatttttttactaatgatacttacaagtttctagctctcacagacatatagaataagggtttttgcataataagcaggttgttatttaggttaacaatattaattcaggttttttagttggaaaaacaattcctgtaaccttctattttctataattgtagtaattgctctacagataatgtctatatattggccaaactggtgcatgacaagtac

SEQ ID NO: 407 - DNA, 447 bp, human
cccggagcgctaggagcgcgcggaaggagccatggctctggacgggataaggatgccagatggctgctacgcggacgggacgtgggaactgagtgtccatgtgacggacctgaaccgcgatgtcaccctgagagtgaccggcgaggtgcacattggaggcgtgatgcttaagctggtggagaaactcgatgtaaaaaaagattggtctgaccatgctctctggtgggaaaagaagagaacttggcttctgaagacacattggaccttagataagtatggtattcaggcagatgctaagcttcagttcacccctcagcacaaactgctccgcctgcagcttcccaacatgaagtatgtgaaggtgaaagtgaatttctctgatagagtcttcaaagctgtttctgacatctgtaagacttttaatatcagacaccccgaagaactttc

SEQ ID NO: 408 - DNA, 129 bp, human
gcagcaggtatgtgcagaggccagaaccaagatgggatttccctgctgaactatgtgagatgctgcatttctatgttgtgtttgctctgatgcagcagtaagtttcttgattcacttagaccagcacat

SEQ ID NO: 409 - DNA, 441 bp, human
ggacaaagagcgctatgtctccctggtgatgcctgtggccctgttcaacctgattgacacttttcccttgagaaaagaagagatggtcctacaagccgaaatgagccagacctgtaacacctgacatgatggttcctctcttggcaattcctcttcattgtctacatagtgacatgcacacgggaaagccttaaaaatatccttgatgtacagattttatttgtaattttaaaagtctattttattatgagctttctttgcacttaaaaattagcatgctgctttttgtacttggaagtgtttcaaaaaattatatgaccatatttactctttctaacctttctttactccatcatggctggttgatttgtagagaaattagaacccataaccatacacaggctatcaacatgttattcaatgtgacacctaactcttttc

SEQ ID NO: 410 - DNA, 538 bp, human
catagctgtgctcaacaacctggtggtggccagccccatcgctgcccgagacctgctgtattttgacggactccgaaagctcatcttcatcaagaagaagcgggacagccccgacagtgagaagtcctcccgggcagcatccagcctcctggccaacctgtggcagtacaacaagctccaccgtgacttccgggcgaagggctatcggaaggaggacttcctgggcccataggtgaagccttctggaggagaaggtgacgtggcccagcgtccaagggacagactcagctccaggctgcttggcagcccagcctggaggagaaggctaatgacggaggggcccctcgctggggcccctgtgtgcatctttgagggtcctgggccaccaggaggggcagggtcttatagctggggacttggcttccgcagggcagggggtggggcagggctcaaggctgctctggtgtatggggtggtgacccagtcacattggcagaggtgggggttggctgtggcctggcagtatcttgggatagcc

SEQ ID NO: 411 - DNA, 464 bp, human
attggaatctcctgtgcatcacatttcttaaaaagagaaatgcagaaaaccaaagaactaatgacaaaagtgaataaacaaaaactgtttgaaaagagtacaggacttcctcacaaagcatcacgtcatcttgacagctatgaattccttaaagccattttaaactgaggcattaagaagaaatgcactcaccatgagcaccaacttctgcatctgcctgatcatatttaaaggaacagagaaatatttgtaattaatctgcccagtaaataccagctcgtagcagttggcaggtgcatgtctagataaaatttcttgcagctaatttaaactttctacacgcaccagtagataatctcaatgtaaataatacatttcttcttggctctttaatgtaagccaacatggagaggaagatcttgacttatattctgtaccacatacacttctgtggacttttagca

SEQ ID NO: 412 - DNA, 461 bp, human
agagcatcctggccacgatgaacgtaccagccgggccagcaggcggccagcaagtggacctggccagtgtgctgacgccggagataatggctcccatcctcgccaacgcggatgtccaggagcgcctgcttccctacttgccatctggggagtcgctgccgcagaccgcggatgagatccagaataccctgacctcgccccagttccagcaggccctgggcatgttcagcgcagccttggcctcggggcagctgggccccctcatgtgccagttcggtctgcctgcagaggctgtggaggccgccaacaagggcgatgtggaagcgtttgccaaagccatgcagaacaacgccaagcccgagcagaaagagggcgacacgaaggacaagaaggacgaagaggaggacatgagcctggactgagccacgcgccgtcctccgaggaactgggcgcttgcagtg

SEQ ID NO: 413 - DNA, 354 bp, human
gaatgtgagagcctgggtgtctgagaccgggagggcccagcagtgaggggcaggctcttctggtcaccaggctgttcagtggactcagttcttcatcttgtaatgtcgatggctttgccacaccaggccaagcccatgccataccttgtcaagactgtcaaagtggttgtggttaggtcaaactggttttggttctgatggttaggaagaaacaggtcagccctcagatcacctggcccgggacagctgaccccctagaaccctggctctgccattagctaggacctaagactctgcccacattttggtctgttctctcccattacacataggtttgtctcagcatgcaagagt

SEQ ID NO: 414 - DNA, 433 bp, human
cagtacttgcccactgacagtcaatatctgtcacctagtaagcaaccaagagaccctcccttcatggcttccgatcagatggcaagggtctttgcagatgtgcattccagagccagccgggattccagtgagatgggtgctgttcttgagcagcttgaccaccccaacagggatctgggcagagagtctgtggatgcagaggaagttgtgagagaaattgataagcttttgcaagactgccggggaaacgaccctgtggctgtgagaaagtgaaaaaagaaaaaaaaaaaaggcattggcattttcttgtctcttctgttgatttaaaaatgatccctcctggtgataacccattttacagggatgaagaaagaccaatgctgctttaaggcttttagtgaacatctgaagtgcccacaagtatgttctttcc

SEQ ID NO: 415 - DNA, 456 bp, human
ctccagcagttctctcgaatactttgaatgttgtgtaacagttagccactgctggtgtttatgtgaacattcctatcaatccaaattccctctggagtttcatgttatgcctgttgcaggcaaatgtaaagtctagaaaataatgcaaatgtcacggctactctatatacttttgcttggttcattttttttcccttttagttaagcatgactttagatgggaagcctgtgtatcgtggagaaacaagagaccaactttttcattccctgcccccaatttcccagactagatttcaagctaattttctttttctgaagcctctaacaaatgatctagttcagaaggaagcaaaatcccttaatctatgtgcaccgttgggaccaatgccttaattaaagaatttaaaaaagttgtaatagagaatatttttggcattcctctcaatgttgtgtgtt

SEQ ID NO: 416 - DNA, 463 bp, human
  misc_feature (37)..(37): n is a, c, g, or t
gctacatgagggtgtccctgtccagctttctggcacntgagtcctgtgtggagagttacctcctcttccagggactgtgctgttgggaactttgggcaagtcacttacctctttgtgcctcaatttctgtataatatttctaagctacctcactgaggtggtatgaagattcactaatgtatgtagcgtgtttgtcaatcctccagtgaaaagcactatctagatcacattttggatcacattagccaaatgcagtaaatggccaaattagatgtgtgctgaagacaatcagtcactgggtctatattaaacagcaaccagagcaacaaatggcaaacaatttctattttcaagtttctttgcatatttttttggtgcaaaaccatttataaacttttttttctaacactagtgtctacagcagcattcaaaaaaattctgttaccttttctgtattaggatt

SEQ ID NO: 417 - DNA, 530 bp, human
cccccatgcaatgagcttctaactcagagctgatgaacaaaagcccccccacccccaatgcctgcctcctcactcctccgtcgctgcccttcacaccttttggtgctacccctccccagagttaagcactggatgtctcctgatcccaggctgggacccctacccccaccccctttgatcctttctacttccacggtgaaaggactgaggtcggactacagagggaagagggacttcccttgactgggttgtgtttcttttcctgcctcagcccagctctgcaaatcccctccccctgcccctcacctccccaggctcaccttgccatgccaggtggtttggggaccaagatgttgggggggtgaatcaggatcctaatggtgctgccctatttatacctgggtctgtattaaaagggaaagtcccccctgttgtagatttcatctgcttcctccttagggaaggctgggatatgatgagagattccagcccaagcccggccccccaccgccaggccatagggcataatt

SEQ ID NO: 418 - DNA, 169 bp, human
aaaaatgcacatagctatcgagtgtgctttagcttgaaaaggtgaccttgcaacttcatgtcaactttctggctcctcaaacagtaggttggcagtaaggcagggtcccatttctcactgagaagattgtgaatatttccatatggattttctattgttactctggttc

SEQ ID NO: 419 - DNA, 472 bp, human
caacctctcggccaagaccgagctggtggcctgggcggtgtccaactggaagccggactcagccagggtgcgctactaccagagcctgcaggctcatctcaaggtggacgtgtacggacgctcccacaagcccctgcccaaggggaccatgatggagacgctgtcccggtacaagttctacctggccttcgagaactccttgcaccccgactacatcaccgagaagctgtggaggaacgccctggaggcctgggccgtgcccgtggtgctgggccccagcagaagcaactacgagaggttcctgccacccgacgccttcatccacgtggacgacttccagagccccaaggacctggcccggtacctgcaggagctggacaaggaccacgcccgctacctgagctactttcgctggcgggagacgctgcggcctcgctccttcagctgggcactggatttctgcaaggcctgc

SEQ ID NO: 420 - DNA, 514 bp, human
gtctacttcatgccaatgagctactcctcaacaggggctggttttgccatttgaggaatgacagccactatgttgtgtacacaagagagctggatggcatcgacagaatctttatcgtggttctgaattttggagaatcaacactgttaaatctacataatatgatttcgggccttcccgctaaaataagaataaggttaagtaccaattctgccgacaaaggcagtaaagttgatacaagtggcatttttctggacaagggagagggactcatctttgaacacaacacgaagaatctccttcatcgccaaacagctttcagagatagatgctttgtttccaatcgagcatgctattccagtgtactgaacatactgtatacctcgtgttaggcacctttatgaagagatgaagacactggcatttcagtgggattgtaagcatttgtaatagcttcatgtacagcatgctgcttggtgaacaatcattaattcttcgatatttctgtagcttg

SEQ ID NO: 421 - DNA, 535 bp, human
ggagctggaactggtcaccaaggccggcttccgggcccttctctctgccccctggtacctgaaccgtatatcctatggccctgactggaaggatttctacgtagtggaacccctggcatttgaaggtacccctgagcagaaggctctggtgattggtggagaggcttgtatgtggggagaatatgtggacaacacaaacctggtccccaggctctggcccagagcaggggctgttgccgaaaggctgtggagcaacaagttgacatctgacctgacatttgcctatgaacgtttgtcacacttccgctgtgagttgctgaggcgaggtgtccaggcccaacccctcaatgtaggcttctgtgagcaggagtttgaacagacctgagccccaggcaccgaggagggtgctggctgtaggtgaatggtagtggagccaggcttccactgcatcctggccaggggacggagccccttgccttcgtgccccttgcctgcgtgcccctgtgcttggagagaaaggggccggtgctggcgc

SEQ ID NO: 422 - DNA, 423 bp, human
aataatttattcccacatctacatcagtgaaagctatctacctatcctgagtctatcttaaaggaaaaaaagaaaaaaaccttatctcttgcccttattttgaattttccactctttcattaatttgttttaagctcctgttggaaaaaaaggggtagtgcattttaaattgaccttcatacgcttttaaaataagacaaatctacttgataatgtacctttatttgatctcaagttgtataaaaccaataaatttgtgttactgcagtagtaatcttatgcacacggtgatttcatgttatatatgcaaagtaggcaactgttttcttagttacagaagtttcaagcttcacttttgtgcagtagaaacaaaagtaggctacagtctgtgccatgttgatgtacagtttctgaaattgtttt

SEQ ID NO: 423 - DNA, 501 bp, human
gcagcttggagtgctaactggaagatcaaaatcatgtctcttgctgataacctaagagcattaagctctgagtccagtgaggtcatagcagtgtatgctccttgcccttacggagtgtctgtttttagaaatttagattttaaaaggcgtgggacatacgaccaggccctatagccacataaatataaatttcatagaaaaagaataaaagcggagatatattttttgacacagaggcacccaaagaaatacaaacattgcctagctgctgccatttctgtaactgcccagaagggtgacagatttctgaaggggaaaggattcagatatgaccttttctttagtcccaaccactagttttaacaaatgtgaattattgaaatgtgaagggacaaaaagaatcatacatttaaactgtcttgttcagcataccaatattgtatgttacaaatcatcatttctaaatctggattgattctgttgtgtttttgactgtttcta

SEQ ID NO: 424 - DNA, 520 bp, human
tgggacctacgcacaggacgttgtatcatgttcttagaaggccacctgaaagaaatctatggaataaatttctcccccaatggctatcacattgcaaccggcagtggtgacaacacctgcaaagtgtgggacctccgacagcggcgttgcgtctacaccatccctgctcatcagaacttagtgactggtgtcaagtttgagcctatccatgggaacttcttgcttactggtgcctatgataacacagccaagatctggacgcacccaggctggtccccgctgaagactctggctggccacgaaggcaaagtgatgggcctagatatttcttccgatgggcagctcatagccacttgctcatatgacaggaccttcaagctgtggatggctgaatagatgacaatgggaaaaggacttgaacctcaagctctctctaaggagctgttttcctcaaacgagaagaattgaagtgtttagttctatcatgttttctgccaattaccatgcatagaccctcagt

SEQ ID NO: 425 - DNA, 159 bp, human
agggtgaaacttcctacatccgagtttatcctgagagaagcaccagctatggctactcacggtctcggtctgggtcaaggggccgtgactctccataccaaagcaggggttccccacactacttctctcctttcaggccctactgagacaggtgatggg

SEQ ID NO: 426 - DNA, 353 bp, human
agaagttctaataatggtcctaatcactgcatttttaaaaaacaaagttcaacacaaatgacatttgttttaaactttagtagataaaaggtgaaccatgtgacatgggcatttttgtaagtcaaaaacaaatttcacatatggtaaacctaatattcacagtgtgttccctcacttgtaatctctgaatacaaatatactagcttttctaaagggaatcatttttttaaaagtagtgccactgacaagatgctacagtgaagattatccattcttaggatatttattttcagtgaacattttctgcacaaaggtagtgttgcactgggacacaagccttttaacagataacc

SEQ ID NO: 427 - DNA, 479 bp, human
  misc_feature (348)..(348): n is a, c, g, or t
tgctttcaaatcattcctcaatcccttgttaacttctggtgtgaggatctctattttccttaaacgttgaaaagcatcataatcagtctctccaaatagtctgattggttctcctctttctctcaatcttctgatgacctcttgcctagaaagcgtcataggtaatttttcctctgccagttcaagttctaacactggattcgatgaagttaatggtttctggtcctcctcttttggctgtatcttgtagccacatctttcaaaatatgcttcctcttcttttttggcgagctcactacgcttgaaatattttttattttccaccagcaggttcctgtcctccaccanctgccgcttccgaaggatctctgatttcagaatgtccatctcgccgccagaatttaagctccagtttcccgctccagctcctcgaacccagcctcactgggccggcggccgcgaacccactgagtatacgg

SEQ ID NO: 428 - DNA, 375 bp, human
aagttcgagttgtgctcttcacgttggttcgataatggcctttatttgaaagctttttaatttttctttacagtaaatattccattctgatttcataaattaaacatttatgcctcccttttgtgttgacactgtagctcatactggaaaagtcgatcaatgttttgcagtttattgaaagtagttctatatataacaatgttataagcatttctttagaaatggttgaaaatgcttctaaaatgtgattatcgaccatggtatgcatgatcgttgtaattgttgacattccttttagaagttgtgaaatgttacaacttgtgcttatgtagacacaattttttgtttcagtaccagaggcactgacttcaataa

SEQ ID NO: 429 - DNA, 566 bp, human
gagagccctgaaactattagcacggagaagccctcacttcttccagccaaccaaccagcagtttaaaagtttacaagaatatcttgaaaatatggtaataaagctagccaaggaattaccgcctccttctgaagaaataaaaacaggtgaggatgaagatgaggaagataatgatgctctactgaaggaaaatgaaagtcctgatgttcggcgagacaaacctgtaacaggagaacaaatagaggtatttgccaacaagctgggtgaacaatggaagattctggctccctacttggaaatgaaagactcagaaattaggcagattgagtgtgacagtgaagacatgaagatgagagctaagcagctcctggttgcctggcaagatcaagagggagttcatgcaacacctgagaatctgattaatgcactgaataagtctggattaagtgaccttgcagaaagtctaactaatgacaatgagacaaatagttagcttcttttttttttctttttattaaaactgtgatagattttgttaccaagcagcatttgataagaggtccact

SEQ ID NO: 430 - DNA, 198 bp, human
gttggctgatattggagtgctcattcacatgaagtggatagatacttctcaagacatcacacagcgtgagtcaatcaaggagggaagccacaagcagactgacaacgtttctagcaggatcaggtgagctgtgtccagaaaaccaacgagaaggagtggaaggaggaatgaacgtttcattctcgttaataaaggcat

SEQ ID NO: 431 - DNA, 410 bp, human
acagcatgacgtccgctttggagaactacatcaaccgaactgttgccgttattacatcagatgggagaatgattgtgggaacactgaaaggttttgaccagaccattaatttgattttggatgaaagccatgaacgagtattcagctcttcacagggggtagaacaagtggtactaggattatacattgtaagaggtgacaacgttgcagtcattggagaaatcgatgaagaaacagattctgcgcttgatttggggaatattcgagcagaacctttaaattctgtagcacactgaggaaaaactacatacttggacatctgtaaatctttgtacagaaactgattattctgaggatgatatatggagtttttatgagtgtgtctctggattttgaccccttattgattc

SEQ ID NO: 432 - DNA, 571 bp, human
gtcatattgtttcctgtgctggagcaaaagtcattacactttgaagtattatattgttcttatcctcaattcaatgtggtgatgaaattgccaggttgtctgatatttctttcagacttcgccagacagattgctgataataaattaggtaagataatttgttgggccatattttaggacaggtaaaataacatcaggttccagttgcttgaattgcaaggctaagaagtactgcccttttgtgtgttagcagtcaaatctattattccactggcgcatcatatgcagtgatatatgcctataatataagccataggttcacaccattttgtttagacaattgtctttttttcaagatgctttgtttctttcatatgaaaaaaatgcattttataaattcagaaagtcatagatttctgaaggcgtcaacgtgcattttatttatggactggtaagtaactgtggtttactagcaggaatatttccaatttctacctttactacatcttttcaacaagtaactttgtagaaatgagccagaagccaaggccctgagttggcagtggcccat

SEQ ID NO: 433 - DNA, 516 bp, human
gaacacatccaaaatgcatgattcttacaaactaaaggaagttaatgtgcaaaaatctcaaccaaaactactatttttagtatacttgtttacgtaaatgctgattggaaatatttaaatgactatagatctgattctttcttttcctttggaaactgggattaatgtatgctctagatccatttattagaaatgcaaaaatactacaattttttgatggatgaaaatactcctgtaacacaaacagagaactggaggaactgaagaataactcactcatatagctctgcctcattctgtgtgtgtgtgcatgtgtgtgttagcagaggtattttactcagaaaataggtttcaaagaacattaatgactttcttttcccttttatgtctgcttaatcagtgttaaactgctatgggaaaagttttatagaactatataacctgaatgttggtctctttgtacacatcttttctatgactgcaaatcttcactttatgtatcatttttactgtcat

SEQ ID NO: 434 - DNA, 488 bp, human
ttcaggcattagcaattcgtcgactagacaaaccaaagaagctatctgctgagtgtgctaaatactggggagaaatcatctcccagcaatataattttgacagagataacactgaggttgcatatttaaagacacttaccaaggaagatatcatcaaattctacaaggaaatgttggcagtagatgctccaaggagacataaggtatccgtccatgttcttgccagggaaatggattcttgtcctgttgttggagagttcccatgtcaaaatgacataaatttgtcacaagcaccagccttgccacaacctgaagtgattcagaacatgaccgaattcaagcgtggtctgccactgtttccccttgtgaaaccacatattaacttcatggctgcaaaactctgaagattccccatgcatgggaaagtgcaagtggatgcattcctgagtcttccagagcctaagaaaatcatcttggccactttaata

SEQ ID NO: 435 - DNA, 527 bp, human
ttatctgggtccttcaacaactggagtaaacttcccctcaccagaagccacaataactttgtagccatcctggatctgccggaaggagagcatcagtacaagttctttgtggatggtcagtggacgcacgacccttccgagcccatagtaaccagccagcttggcacagttaacaacatcattcaagtgaagaaaactgactttgaagtatttgatgctttaatggtggattcccaaaagtgctccgatgtgtctgagctgtccagttctcccccaggaccctaccatcaggagccctacgtctgcaaacccgaagagcgctttcgggcaccccctattctccccccacatctcctccaggtcatcctgaacaaggacacggggatttcctgtgatccagctttgcttcctgagcccaatcacgtcatgctgaaccacctatacgcgctgtctatcaaggatggagtgatggtgctcagcgcaacccaccggtacaagaagaagtacgtcaccaccttgttatacaa

SEQ ID NO: 436 - DNA, 465 bp, human
  misc_feature (420)..(420): n is a, c, g, or t
ttcaggcattagcaattcgtcgactagacaaaccaaagaagctatctgctgagtgtgctaaatactggggagaaatcatctcccagcaatataattttgacagagataacactgaggttgcatatttaaagacacttaccaaggaagatatcatcaaattctacaaggaaatgttggcagtagatgctccaaggagacataaggtatccgtccatgttcttgccagggaaatggattcttgtcctgttgttggagagttcccatgtcaaaatgacataaatttgtcacaagcaccagccttgccacaacctgaagtgattcagaacatgaccgaattcaagcgtggtctgccactgtttccccttgtgaaaccacatattaacttcatggctgcaaaactctgaagattccccatgcatngggaaagtgcaagtggatgcattcctgagtcttccagagcctaag

SEQ ID NO: 437 - DNA, 218 bp, human
gggggtaaagtctttcaccagcacacaagagtttgattgtacaaatatatcttctgcattaacatctctgcctgttgcttaagatcagttgcttttatactcagaatggaaatacctgatcttggctagttttgttataagatattgatttcatttagatttccctccacgaggtcagcaaactatcatgttcttatgtaaacttaggccaaggccag

SEQ ID NO: 438 - DNA, 483 bp, human
cggttcaccttgccgagagaggcgcgtctgccccaccctcaaaccctgtggggcctgatggtgctcacgactcttcctgcaaagggaactgaagacctccacattaagtggctttttaacatgaaaaacacggcagctgtagctcccgagctactctcttgccagcattttcacattttgcctttctcgtggtagaagccagtacagagaaattctgtggtgggaacattcgaggtgtcaccctgcagagctatggtgaggtgtggataaggcttaggtgccaggctgtaagcattctgagctggcttgttgtttttaagtcctgtatatgtatgtagtagtttgggtgtgtatatatagtagcatttcaaaatggacgtactggtttaacctcctatccttggagagcagctggctctccaccttgttacacattatgttagagaggtagcgagctgctctgctatgtccttaagccaatat

SEQ ID NO: 439 - DNA, 158 bp, human
ccttcctggagcagtcgaaaacccgatttaacaagaactacaaggggatgagtctatccaaaatcaccaccaccgtgggcctaaacatcggcactgtggatgtgggaaaggctcggctcatgttctgggacttaggagggcaggaagagctgcagtct

SEQ ID NO: 440 - DNA, 533 bp, human
gccagatcgtcagcgctaagggaactgagaccagagaaagaacccaagagaactaaagttatgtcagctacccagacttaatgggccagagccatgaccctcacaggtcttgtgttagttgtatctgaaactgttatgtatctctctaccttctggaaaacagggctggtattcctacccaggaacctcctttgagcatagagttagcaaccatgcttctcattcccttgactcatgtcttgccaggatggttagatacacagcatgttgatttggtcactaaaaagaagaaaaggactaacaagcttcacttttatgaacaactattttgagaacatgcacaatagtatgtttttattactggtttaatggagtaatggtacttttattctttcttgatagaaacctgcttacatttaaccaagcttctattatgcctttttctaacacagactttcttcactgtctttcatttaaaaagaaattaatgctcttaagatatatattttacgtagtgctgacaggacccactc

SEQ ID NO: 441 - DNA, 331 bp, human
cagcactggtcctatggactgcgccctggaggaaagagagatgccgaaaatttgattgattctttccaagagatagtcaaagaggttggtcaactggcagaaacccaacgcttcgaatgcaccacgcaccagccacgttctcccctccgagacctgaaaggagctctggaaagtctgattgaagaggaaactgggcagaagaagatttaaatccattgggccagaaggaatgaccattactaacatgacttaagtataattctgacattgaaaatttataacccattaaatacctgtaaatggtatgaatttcagaaatccttacaccaag

SEQ ID NO: 442 - DNA, 499 bp, human
cgagcccggttctggggcgaaaatgcctgcccttcacatcgaagatttgccagagaaggaaaaactgaaaatggaagttgagcagcttcgcaaagaagtgaagttgcagagacaacaagtgtctaaatgttctgaagaaataaagaactatattgaagaacgttctggagaggatcctctagtaaagggaattccagaagacaagaacccctttaaagaaaaaggcagctgtgttatttcataaataacttgggagaaactgcatcctaagtggaagaactagtttgttttagttttcccagataaaaccaacatgctttttaaggaaggaagaatgaaattaaaaggagactttcttaagcaccatatagatagggttatgtataaaagcatatgtgctactcatctttgctcactatgcagtcttttttaagagagcagagagtatcagatgtacaattatggaaataagaacattacttgagcatgacacttcttt

SEQ ID NO: 443 - DNA, 463 bp, human
ggggtctcattagctttgcaacaggaaacatcctgttttattatggtagtggggtcaggaatgtaggaactggtatccattctgccaattcccacccattcagtttgcttatccctacagaacagtgactgaggttcctttttttttttttttttttttttttttcaaatttccatgtattttctgccatttttcagggtctaagattggtcatacattcccaatttactctcagttccagtcaagctggttgctctgaaagtaacccagcttgttgctctaaaatacctcagtagcctgagtgttatactagagatctaaagggttaacaggatagggtggaaaggttagagactcctagaaatctctggtcaccgtgatcttcggcctcattctaatacctgttctttggacagtctttttcctttggtgctctcttgcctttagctaccttctctaatat

SEQ ID NO: 444 - DNA, 521 bp, human
gagatcattctgcactaagtttcgggaacttatccccgacagtgactgaactcactgactaataacttcatttatgaatcttctcacttgtccctttgtctgccaacctgtgtgccttttttgtaaaacattttcatgtctttaaaatgcctgttgaatacctggagtttagtatcaacttctacacagataagctttcaaagttgacaaacttttttgactctttctggaaaagggaaagaaaatagtcttccttctttcttgggcaatatccttcactttactacagttacttttgcaaacagacagaaaggatacacttctaaccacattttacttccttcccctgttgtccagtccaactccacagtcactcttaaaacttctctctgtttgcctgcctccaacagtacttttaactttttgctgtaaacagaataaaattgaacaaattagggggtagaaaggagcagtggtgtcgttcaccgtgagagtctgcatagaactcagcagtgtgccct

SEQ ID NO: 445 - DNA, 448 bp, human
gacagattgttccctcaattcttttccttttccttggcctaaagcaggtctgtgctactagacaactggtaaaccgggaagatcgttcaaaagcagagaaagctgatatggaagaaaatgaggagatttcaagtgatgaagaggagacaaatgtaactgctcaagcaatgcagtcaaataatggaagaggtgaagatgaggaggaggaagatgatgactgggatgaagaagtattggaagaaaccgcgcttgaggggttcagtactccacttgaccttgacaatagtgtggatgaatatcagttttttacacaagctctgataactgtgcagagtcgagatgcagcctggtaccagctgctgatggcaccactcagcgaggatcagaggacagcactgcaggaggtgtacacactggcagagcaccgacggacggtggcagaggcaaa

SEQ ID NO: 446 - DNA, 458 bp, human
cgctgccgtggatacttagttcgacaggaattccgatccaggatgaatttcctgaagaaacaaatccctgccatcacctgcattcagtcacagtggagaggatacaagcagaagaaggcatatcaagatcggttagcttacctgcgctcccacaaagatgaagttgtaaagattcagtccctggcaaggatgcaccaagctcgaaagcgctatcgagatcgcctgcagtacttccgggaccatataaatgacattatcaaaatccaggcttttattcgggcaaacaaagctcgggatgactacaagactctcatcaatgctgaggatcctcctatggttgtggtccgaaaatttgtccacctgctggaccaaagtgaccaggattttcaggaggagcttgaccttatgaagatgcgggaagaggttatcaccctcattcgttctaaccagcagctgga

SEQ ID NO: 447 - DNA, 514 bp, human
tattgcatactacacagaagcttcaaattcctcatgttttaaatgagcactagagggaggtttatcacctttttggttaccagatgataatctatatttgttattactcagtgactctctaatttcacatcagcatgttcagcttgtgctagtcatcattctgatttgcttatctgtttttggtcaaaatatttatttttaaacatgttagaacttaccaaaatgaaagcaacagtgatgtattgaaatcttagttttgatattttaaagaaatgactttctagattaaaaaaatagttttgtagcattttaaacttagtgactatttagttcaattgttcatccattttttatttgcttttataattgcctccttgttttggtatattgtaaaataatttaaataatgtatttataaatatgtataattatgatgtaaaataccgtgtgtatatgtatgtatgtgtgtttctgtatatgcacacacataaacggctttgcctagccaaggatt

SEQ ID NO: 448 - DNA, 468 bp, human
accaagccagttggaagttccagccacagaagaaataaaagagactgatggaagctctcagatcaagcaagaaccagaccccacgtggtagacctcttccctcctaggcttaaagtatcagtggttgagaagagcttttcggacctgttactaccccaagctgtgtaatatacttgtataacagaaataccttctatacaaacctttttttctacttttagatagaaatgtctactttttcagcagttctgtgaattaaagagcagagtgactgtgggtctggaatggctggtgtacttgggaatgtactatcaggattttacagcaatgctgggaaatgacagggaaaatgacaggaatgaatctcaccagattttttatgtactcagcagagccttgagttacggtgtttattttccaatcaagtgaagatatctcctacttctcctactggaacatctcagcttc

SEQ ID NO: 449 - DNA, 308 bp, human
  misc_feature (170)..(204): n is a, c, g, or t
cactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaannnnnnnnnnnnnnnnnnnnnnnnnnnnnnnnnnnggcacaaacaagactgatgttgtctgtggtccccaggntcggctgagagccctggtggtgatccccatcatcttcgggntcctgtttgccatcctcttggtgct

SEQ ID NO: 450 - DNA, 470 bp, human
  misc_feature (320)..(320): n is a, c, g, or t
gtcagtggcctggatcatagccctggatcatttcccagtctgtcctgtgctgtgtgaccttggacaaggcgcttcatctctctgggcctctatttctccatttgtaaaacaagtggctgcagtagatgatggctgagagcccttcctgttcccagatgccttggtccaaagaccccacccctctgctggtcctgccaacgtgttggtgctataagctgcttcagatataaaattggtttatctataatgtttgttcatttaatagcttctaaaaggcctttttgttatacagtgctttttttctagttttatggacttgnttactgtaataatgtcttgtttttagccatgtaactacaaacagatattctcttgatgtcttagtaaatttgcatttgatatatcattgatgagattttgttgttatgtaatattctttgnntacgcatctgtccagcatcttattaacc

SEQ ID NO: 451 - DNA, 475 bp, human
agttgaagatggtcccttacagcttcccaagttaggttagtgatgtgaaatgctcctgtccctggccctacctccttccctgtccccacccctgcataaggcagttgttggttttcttccccaattcttttccaagtaggttttgtttaccctactccccaaatccctgagccagaagtggggtgcttatactcccaaaccttgagtgtccagccttcccctgttgtttttagtctcttgtgctgtgcctagtggcacctgggctggggaggacactgccccgtctaggtttttataaatgtcttactcaagttcaaacctccagcctgtgaatcaactgtgtctcttttttgacttggtaagcaagtattaggctttggggtggggggaggtctgtaatgtgaaacaacttcttgtctttttttctcccactgttgtaaataacttttaatggccaaaccccagatttgtactt

SEQ ID NO: 452 - DNA, 528 bp, human
gactagctatctctgtggggtctattttataagggcactaaccccattcatgagagcagagcccccatggcctaatcacctttccaaggccccaccttctatctaagacaatcacgctgggaataggtttcaacatatgaattgggggaggacacatttggaccacagcatgaacctttagaacagggtttctcagccttagcactacggacattttgggctggataaatatgtgttggtacagaatgggggtatcctgtgcattgtaggatctttagcagtaccctagcctcaactcactagatgccaatgacataccttgcttcttcaccagttatgataaccaagaatgtctccattgttaaatgtccccttaggagcaaaattgcccctggttgagaaacattgctttagacaaattgttaagagtatcatgtactacacttctgaaacttaacgtgatcatcaccactgacagatgattcacagagagagactgtttgaatcttgtctcactagtttttcctg

SEQ ID NO: 453 - DNA, 424 bp, human
agttggatcactgcattttcacacagagaaaaatcactggcccttctcagaggagcaagagaccctcaacagataccagcgtgtgtatagaacttccaaatgctgagcccagagcgttgtctcctgcccatgagcaccacagtcaggccttgatgggatcttctagggagacaacagccctgtctcaaacccagcttggcagctctaatgtaccagcagctggaatctgaaggcgtgagtctccatcttagagcatcactcttcctcacaccacaaatctggtgcctgtctcttgcttaccaatgtctaaggtccccactgcctgctgcagagaaaacacactcctttgcttagcccacaattctctatttcacttgacccctgcccacctctccaacctaactggcttacttcctagtctact

SEQ ID NO: 454 - DNA, 502 bp, human
ccacccacggagagacctacagatgcttcggctctttccatggatctccctacgagtggtcagacccgagtgacccactgcctgtttctgtcacaggaaacccttctagtagttggccttcacccactgaaccaagcttcaaaactggtatcgccagacacctgcatgctgtgattaggtactcagtggccatcatcctctttaccatccttcccttctttctccttcatcgctggtgctccaaaaaaaaaatgctgctgtaatgaaccaagagcctgcgggacacagaacagtgaacagggaggactctgatgaacaagaccctcaggaggtgacatacgcacagttggatcactgcattttcacacagagaaaaatcactggcccttctcagaggagcaagagaccctcaacagataccagcgtgtgtatagaacttccaaatgctgagcccagtgcgttgtctcctgcccatgagcaccacagtcaggccttgatggga

SEQ ID NO: 455 - DNA, 302 bp, human
gctgtgggtttcatcatggcaggcacacctttggttctcatcatcactgcctacagccacgtggcagctgcagttctacgaatccgttcagtggagggccgaaagaaggccttctccacgtgtggctcccacctcaccgtggtttgtcttttctttggaagaggtatcttcaactacatgagactgggttcagaggaggcttcagacaaggataaaggggttggagttttcaacactgttatcaaccctatgctgaaccctcttatctacagcctcagaaaccctgatgttcagggtgctct

SEQ ID NO: 456 - DNA, 547 bp, human
gcaaaggggtttcctcgatttggagctttttttttcttccaccgtcatttctaactcttaaaaccaactcagttccatcatggtgatgttcaagaagatcaagtcttttgaggtggtctttaacgaccctgaaaaggtgtacggcagtggcgagaaggtggctggccgggtgatagtggaggtgtgtgaagttactcgtgtcaaagccgttaggatcctggcttgcggagtggctaaagtgctttggatgcagggatcccagcagtgcaaacagacttcggagtacctgcgctatgaagacacgcttcttctggaagaccagccaacaggtgagaatgagatggtgatcatgagacctggaaacaaatatgagtacaagttcggctttgagcttcctcaggggcctctgggaacatccttcaaaggaaaatatgggtgtgtagactactgggtgaaggcttttcttgaccgcccgagccagccaactcaagagacaaagaaaaactttgaagtagtggatctggtggatgtcaatacccctgattta

SEQ ID NO: 457 - DNA, 566 bp, human
gaacaagcttttacagacgtccttaaggcaacagtgcagagatgtaatccttaaaagaactgggaatggcaaaactactgtcggttgatgtgtcctgaaaattattggagttatggcagaagtgcttttttgatcaactggtttgtgttttgctgctgcatttatcccaagaaaaacagctttaatctccagaagaaaaccaaaataccatgggatttatgctgtattgacatcttgccctaaacgtacaacatcatagtaatttgtcatgggcaacatgaccagagagaagatttttgtcatgattttaaatacactgacacgctactgttggttaaatttaaacatgttttacctgcagaaattctctcacaaataacctgcaataacttgaaatgcatacccttttgaacattccttttctcatgtataaattaaaatgtttgctgcattttgcaaaatgtcaattctctaaaaatgtgtccgtatatttctgtacctgcagtgtagtaaaggtttagacgaaaccccataattatagtggcatactgtcacttaggtttcaa

SEQ ID NO: 458 - DNA, 40 bp, human
tgaaggctcagaagcatcctcctgctcatctgctttggct

SEQ ID NO: 459 - DNA, 373 bp, human
atgaagcttatggccaggacgactggaatgggaccaggccgtcgctgaaggcccctcctgctaggccagtgaagggagcatacagagagcacccatatggacgttattaaaaacaaacatgaggggaaaatatcagttatgagcaaagttgttactgatttcttgtatctcccaggattcctgttgctttacccacaacagacaagtaattgtctaagtgtttttcttcgtggtccccttcttctccccaccttattccattcttaactctgcattctggcttctgtatgtagtattttaaaatgagttaaaatagatttaggaatattgaattaattttttaagtgtgtagatgcttttttctttgttgttt

SEQ ID NO: 460 - DNA, 528 bp, human
  misc_feature (40)..(40): n is a, c, g, or t
gtagtacatacctgaccgggagtccaaaccaccttggtgnctctgaagtccactgactcatcacacctttcttagcctggctcctctcaagggcattctgggcttgtaaacagacataggaagcctctgtttaccctgaagcaccactgtccagcccattggttcccactggcagcatggtagagctgagagaaacaggctctcagggtacctgacttgaggggaatcgtttcatgaagctgaacttcaagcatatttccagtacattctttcagagtctgtttttccatccaaatataagccccaggccattccacttagtgtcttttcaatgataggcaagaatgatatctgagttgaacttcggtgcttctgttgtttgagtttactgtgcctggtggtatattgggcattctttggattgagtgttctgaggtgagagagtcttcccgaggcatcctgtctgtgcttccaaccctgaacaagaccttacatgagagatggactgatggactgcggcaatcctgg

SEQ ID NO: 461 - DNA, 427 bp, human
aagagaggaccatgagaattttaataaaacagaaggaaactaatggaccttccaggatttattgtggacggatgtggatatattctgtacaggaacaacacatatggaagtggactgaagcctatgtagaaacacacacacactgaacattgttattcattttgtaaaatactagtctttattttcattttttgtaaaatttaaacatcgtatgcgcataaagaaaaaggaaacaagaattaggggaaaataacattttccaaataattataaaaaattgtcctgtgtctatgtatctatatctgttttgtatttttttctggttccaaaccagatttcctgtgattctatactaataatttttgatataaccctttgcttcttataatgagtgcgatatatgttgtcgaggctgttcttcaagaat

SEQ ID NO: 462 - DNA, 348 bp, human
gacaaagtgcacttcacttggctccatgatctatttcttagaaagactaaacaaaagtcaagtcctacggtgtcgcgaggggtcagagggtttcaccctgtaacgtccggcccttttggtcccccttggggcagcatccctttcccaggtgtgtgctggacctgactctcggtcactgtgaacccgaccaaacactctaggaaggtctgtcatgctatgtctgctttcaaggtcacgtttggtcactaagacttaggcattcctcaccatgttttaagagctgtaaacaacgttgaacaataacattccacaggccatttcttgattacagggatgactctaccaaca

SEQ ID NO: 463 - DNA, 492 bp, human
  misc_feature (455)..(455): n is a, c, g, or t
ttttctgctctgtagtccaaaaacatttctttaaaagccagaaaatctgtgaaggtgagcagcatgtcgaatatgtcaccagccacttcatccttatggtgctgtaatgttgtggtgaaggctgccatgttgaactcaggaatccgctgcagcagctgttcttcaatgtatttttctaccaaagaaatgtattcattaaaaataggtgtgtagatgagtttattctcttctgtgtcttcaaactccaggtagtacttgtccatgaaatttctctgtaataactggaactcgtcatccatgataatgtcctctaaatatccaaccacagcatcaaattctgcatcagaggcggaggagaaagacagcgcaaagctctctccttctaaggcgtccatcgcagtcgccccgagtaggctccaaccccgcccgcggcccaactcgcatgcagggcgcgngccgcncggcggggttaaggcctctcagccaaggccg

SEQ ID NO: 464 - DNA, 388 bp, human
atactgaggtggatccctgcatcctcaacaacaatgtgcagtgagcatgtggaagaaaagaagcagctttacctacttgtttctttttgtctctcttcctggacactcactttttcagagactcaacagtctctgcaatggagtgtgggtccaccttagcctctgacttcctaatgtaggaggtggtcagcaggcaatctcctgggccttaaaggatgcggactcatcctcagccagcgcccatgttgtgatacaggggtgtttgttggatgggtttaaaaataactagaaaaactcaggcccatccattttctcagatctccttgaaaattgaggccttttcgatagtttcgggtcaggtaaaaatggcctcctggcgtaagctttt

SEQ ID NO: 465 - DNA, 495 bp, human
ttcctaccttctctgggggatttgagggtagaggcaggggaagatctgttgttgcagttgcttctgcccccttgatccaaatgaccatcatctctgatggagatgggttgggtacctggccttcatggcaccttcactgctagggatgctcaaggggcaggcctggggcccttccctcctgtctcttctcggtctttcctctctgagcagcctcctacctcccctgcctgagccctcactccacagccctcccaggtacctagcagaggctgtcagtccttggctcacctggaacagggctggggctgggttggaacaggtgtgtgcccccaccacagctctatgactctgttctccctccctgccattgtggactcttgtatttgagggacctcaagagagtgaggaccctaccatccactgtccatattcagtcccagccccagtgcgcttcctctgttccctccctcagccatccaattcttgagttttctc

SEQ ID NO: 466 - DNA, 500 bp, human
ggcactacataattgctgttctgctgaatcaaatctcttccacatgggtgcatttgtagctctggacctgtctctacctaaggacaagacactgaggagatactgaacattttgcaaaacttatcacgcctacttaagagtgctgtgtaacccccagttcaagacttagctcctgttgtcatgacggggacagagtgagggaatggtagttaaggcttcttttttgcccccagatacatggtgatggttagcatatggtgcttaaaaggttaaatttcaagcaaaatgcttacagggctaggcagtaccaaagtaactgaattatttcaggaaggtcttcaatcttaaaacaaattcattattctttttcagttttacctcttctctctcagttctacactgatacacttgaaggaccatttactgtttttttctgtagcaccagagaatccatccaaagttccctatgaaaaatgtgttccattgccatagctgactac

SEQ ID NO: 467 - DNA, 235 bp, human
gtgcatgtgaccacaaatgcttgcttggacttgcccatctagcactttggaaatcagtatttaaatgccaaataatcttccaggtagtgctgcttctgaagttatctcttaatcctcttaagtaatttggtgtctgtccagaaaaagtcgatttatgtgtattaattggccatcatgatgttatcatatcttattcccttttatgctatgatttattctatcttttgtatttcag

SEQ ID NO: 468 - DNA, 554 bp, human
  misc_feature (26)..(26): n is a, c, g, or t
cttctgtctccaggaagggcagcggncctcccatcactggaagtctgcagtgggggtcgctgggggtggagagaacactaagaggtgaacatgtatgagtgtgtgcacgcgtgtgagtgtgcanntgtgtgtgtgtgcaaaggtccagccaccccgtcctccagcctgcnaaggggtgtctggcgccttgcctgnnnacccangccccctctccccctgagccattgtgggggtcgatcatgaatgtccgaagagtggccttttcccgtagccctgcgccccctttctgtggctggatggggagacaggtcagggccccccaccctctccagcccctgcagcaaatgactactgcacctggacagcctcctcttttctagaagtctatttatattgtcattttataacactctagcccctgcccttattgggggacagatggtccctgtcctgcggggtggccctggcagannnnnnnnnnnnnnnnnnnnnnnnnnnnnnnnnnnnnnnagccccagcccgcccacccctgcctcgagttagttttacaatta

SEQ ID NO: 469 - DNA, 219 bp, human
gacttaatgatgtcttattgggcataaaactgagtgtcccaaaggtttattaataacagtagtagttatgtgtacaggtaatgtatcatgatccagtatcacagtattgtgctgtttatatacatttttagtttgcatagatgaggtgtgtgtgtgcgctgcttcttgatctaggcaaacctttataaagttgcagtacctaatctgttattcccactt

SEQ ID NO: 470 - DNA, 481 bp, human
tgagggccacgggcttgggtagtggaaagggtgtttgggaaattgttaaatcagttacccgtagtagagctatttcttgtacttctaagttttctagaagtggaaggattgtagtcatcctgaaaatgggtttacttcaaaatccctcagccttgttcttcacgactgtctatactgagagtgtcatgtttccacaaagggctgacacctgagcctggattttcactcatccctgagaagccctttccagtagggtgggcaattcccaacttccttgccacaagcttcccaggctttctcccctggaaaactccagcttgagtcccagatacactcatgggctgccctgggcagccagcattcattgtaagttccctctttgaaaactggtgtgtgggtgttcagttctgtgtctggtgggtatggacagacagtaatctcctgtgatctgtgctagctgtgaggcagctctggaacgtga

SEQ ID NO: 471 - DNA, 452 bp, human
gattaacacatgctcacaaccatcctggtcattctcgaacatgttctattttttaactaatcacccctgatatatttttatttttatatatccagttttcatttttttacgtcttgcctataagctaatatcataaataaggttgtttaagacgtgcttcaaatatccatattaaccactatttttcaaggaagtatggaaaagtacactctgtcactttgtcactcgatgtcattccaaagttattgcctactaagtaatgactgtcatgaaagcagcattgaaataatttgtttaaagggggcactcttttaaacgggaagaaaatttccgcttcctggtcttatcatggacaatttgggctataggcatgaaggaagtgggattacctcaggaagtcaccttttcttgattccagaaacatatgggctgataaacccggggtgacctca

SEQ ID NO: 472 - DNA, 509 bp, human
agttttctaagtacgtctgtagcatgatggtatagattttcttgtttcagtgctttgggacagattttatattatgtcaattgatcaggttaaaattttcagtgtgtagttggcagatattttcaaaattacaatgcatttatggtgtctgggggcaggggaacatcagaaaggttaaattgggcaaaaatgcgtaagtcacaagaatttggatggtgcagttaatgttgaagttacagcatttcagattttattgtcagatatttagatgtttgttacatttttaaaaattgctcttaatttttaaactctcaatacaatatattttgaccttaccattattccagagattcagtattaaaaaaaaaaaaaattacactgtggtagtggcatttaaacaatataatatattctaaacacaatgaaatagggaatataatgtatgaactttttgcattggcttgaagcaatataatatattgtaaacaaaacacagctcttacctaata

SEQ ID NO: 473 - DNA, 458 bp, human
tcaccaagtctggcggctcagatcgcaccattgcctacgaaaacaaagccctgatgctctgcgaagggctcttcgtggcagacgtcaccgatttcgagggctggaaggctgcgattcccagtgccctggacaccaacagctcgaagagcacctcctccttcccctgcccggcagggcacttcaacggcttccgcacggtcatccgccccttctacctgaccaactcctcaggtgtggactagacgcgtggccaagggtggtgagaaccggagaaccccaggacgccctcactgcaggctcccctcctcggcttccttcctctctgcaatgaccttcaacaaccggccaccagatgtcgccctactcacctgaggctcagcttcaagaaattactggaaggcttccactagggtccaccaggagttctcccaccacctcaccagtttccaggtggtaag

SEQ ID NO: 474 - DNA, 447 bp, human
gttggttctgaaacagcctcttagcattcagattgtattttaaatacttaaacacaccaattgtagaaagcaaccctttatttttagcaagtttggtttactcttctcccatgagattttgtctttctacattaactagtaagacataaagatttgaaactggaactatattatactttttaaccaatctctgtggcttcactcatgaaatttacttcagttaatatgagactgggggttaagttgtaactaatatgcaaaattgctttgtatatttggtgattttgtaatgtaagtgaattgattcttatttcactgatactattaatcatgcagtgtacaattcaaaatcatgcaatgtttcactttagaattggatttgaagaactcgactttatgtgatcatggtattggtatacatgtggggtggagaacttattttttcat

SEQ ID NO: 475 - DNA, 553 bp, human
ttctcaagaccctgtcggtgatcatgggggtcttcgtgtgttgctggctgcccttcttcatccttaactgcatggtccctttctgcagtggacaccctgaaggccctccggccggcttcccctgcgtcagtgagaccaccttcgacgtcttcgtctggttcggctgggctaactcctcactcaaccccgtcatctatgccttcaacgccgactttcagaaggtgtttgcccagctgctggggtgcagccacttctgctcccgcacgccggtggagacggtgaacatcagcaatgagctcatctcctacaaccaagacatcgtcttccacaaggaaatcgcagctgcctacatccacatgatgcccaacgccgttacccccggcaaccgggaggtggacaacgacgaggaggagggtcctttcgatcgcatgttccagatctatcagacgtccccagatggtgaccctgttgctgagtctgtctgggagctggactgcgagggggagatttctttagacaaaataacacctttcaccccgaatggattccatta

SEQ ID NO: 476 - DNA, 317 bp, human
gggtgcaggtaacccttggtctgtaagcaccaccgatccagggatcattgtctaaataggttactattgtttgtttcatcttgcttttgcatttttattttttaatttccaaattttaagtgttccctctttggggcaaattcttataaaaatgtttattgtaaagttatatattttgtctacgatgggattatgcacttcccaattgggattttacatctggatttttagtcattctaaaaaacacctaattattaaaacatttatagagtgcctactgtatgcatgagttgagttgcttctgaggtacattttga

SEQ ID NO: 477 - DNA, 511 bp, human
  misc_feature (424)..(424): n is a, c, g, or t
gaatgtcagtccaatctccaatgagaacatgagcaaatagacctttccaggttgaaagtgaaacatactgggtttctgtaagtttttcctcatggcttcatctctatctttactttctcttgaatatgctacacaaagttctttattactacatactaaagtttgcattccagggatattgactgtacatatttatgtatatgtaccatgttgttacatgtaaacaaacttcaatttgaagtgcagctattatgtggtatccatgtgtatcgaccatgtgccatatatcaattatggtcactagaaagtctctttatgatactttttattgtactgtttttcatttcacttgcaaaattttgcagaattcctcctttctacccataaattacatataatttttcttctttagtcatggagaacncccccccatcatctcanccctattanctttcccatgtgtactggtattattaaaaagacatttacatacgcaagtttttcactgaca

SEQ ID NO: 478 - DNA, 213 bp, human
ggatcattcttgctgcttaagatgaaaagcattggttttttaaaattagagaataaaatatgtatttaaatttttggtgtgttcacataaagggatgtagctaaaatgttttcataggctattatatattctcgcagcatttccagttaagaggatattaggtatataattctcttcttaaccgaatgtcagatggtcttacgccacagggtg

SEQ ID NO: 479 - DNA, 473 bp, human
gttccccatgtttatgaaagtcctgcacagtttcaaaggcatggtaaataatatatcaatgtttatgtagtctgttacagaaacagctatagataacattatccagtgaagagcaaaattcaagctttagaaaatattcatgcatgcaattttgacatatctaaaaataggtttttgtatatttatggtgggaggtggttgggaacttttaacaaaatggggtgttaatttttgtacagtctgtgggcatttacacatttttaatgtattaaaatttggtaattatgtgtacattaaattaataaaagttacttctagttatgatttgtgaattccctaagaccttggatttttttaagtaactttatatcagaaatgatactgcatctttatatttttaaaattgtattgctgctcaagaatggtaccctcttgtcaaaaaggcatacattcataattgtacattcagcatt

SEQ ID NO: 480 - DNA, 542 bp, human
tactcatttctatttgttactcctttttactggacatgatgtttacaagatacaactgtgttactgtattccatctagtacggggcctttggtgtggcttactatttcattgtgtgcacccacccacccaccacactggacttttctagagatggacagcttggttacctccaccttcctgcactcattctcaaacatactgatgttcatacaaaccagcagagtgctgagggacgatatgtactattacaaaaccagacacttttacattcatggtccaacagaacacatggcctagaggcaatgttgcatataccttaatctttgatatgaataatatctttgttctttatatttcttaaaacagaaagggtggaaaatcactatacagaagcaatatccaaagatctcctgatcataaagacaaggggtcttttcagtcttccctctcctcaaaccttgtgtagcattgcacaatatagatctcagtcaacattcactgagtgccaagaatgtgagaaacactgtaccatgcctgtcat

SEQ ID NO: 481 - DNA, 529 bp, human
cctcccttcctggagggatggccagggaaggagaaaacagagaactgacacctttgaaaccacagaatgtgttacatgcagactcgctcaagggcataagttattgtgaacgtttttgccaatcactgctcaacagccctgctagattttgtatgatgctgaattattatgcagactaattccacccagttgagacacaccatgcttgttcacttgtatttattgaaactgtggattcttgcccgtgctgtcccttgtatttactttaagcactgatcacttatcattcattcggtatggttttccctgtcccttgtacacattctggtatgaatttgtaaaaataacctgctacaaattggttgaatgtttctgtctgtggtgcgaaccagcattaacggatggggcacgtgcccaactgaggaacaggagaagaaatcaccaatttgggctctcagagctaagacacacttattgattctgttgcacattttgcactggtttatggcgattgttttcttggacggat

SEQ ID NO: 482 - DNA, 544 bp, human
tagcagaagcaagacacaacccaagcataggggagggaagtgttggtgggctgactggcagtttgagtgcaagtggaagccacatggatcgaataggagccatccgagacaacctgagtgaaacggccagcaccatggcactagctggagccagtataacggggagtctgtcaggaagtgccatggtaaactgttttaacaggttggaagtacaagcagatgtacagaaagaggctgctgtcgccgctgctgggagatgggcctactcgcccgccaccctgcgctgccgccgctctgaggagttaaagaacatccatgatagcagtcttctgtaaatcgaacttttcaagaattctctgaaggaaccaagtaggatattcttacatcatgacttaatgtgaatgcaagaacaagaaataggttttatctctaaatataatgaagggctgtgtgtaaacactgaccctgtctcaattctaacaagcattttagacatgagtttacaccggcaaatgggttcagatcgagatcttcagtcctctgc

SEQ ID NO: 483 - DNA, 477 bp, human
gatcaggagcaatgccactgctagcataccttccttagtgaaaaatcttttggaaaaggatcccactctgacctgtgaagtactaatgaatgctgttgctacagagtatgctgcctatcatcaaattgataatcacattcacctaataaacccaacggatgagacactgtttcctggaataaatagcaaagccaaagaactgcaaacttgggagtggatatatggcaaaactccaaagtttagtataaatacttcctttcatgtgttatatgaacagtcacacttggaaattaaagtattcatagacataaagaatggaagaattgaaatttgtaatattgaagcacctgatcattggttgccattggaaatacgtgacaaattaaattcaagtcttattggcagtaagttttgcccaactgaaactaccatgctaacaaatatattacttagaacatgtccacaagaccacaaact

SEQ ID NO: 484 - DNA, 281 bp, human
acctacactctctacttaatcattggagatgccactttgaagaacccaatcctctggaatgtggctgatgtggtcatcaagttccctgaggaagaagctccctcgactgtcttgtcccagaaccttttcactccaaaacaggaaattcagcacctgttccgcgagcctgagaagaggccccccaccgtggtgtccaatacattcactgccctgatcctctcgccgttgcttctgctcttcgctctgtggatccggattggtgccaatgtctccaacttcac

SEQ ID NO: 485 - DNA, 213 bp, human
tttattctgaagtcctgttattctgactacagttctttgtgtatacttctgtgtctgttatgttcaataactgagctaacataaaataactctaggtttctacttgatttttcccccatgtatacctttcatctgttctatagcaagttgatgtaaattggtttgtcaacaagaatgttaactgatgaaagtggatagaacccatacatgaat

SEQ ID NO: 486 - DNA, 556 bp, human
gtaccagcatcgcgaaccaggctgtgcgcatccaggagggccggtaccgccaccccggctgctacacctgtgccgactgtgggctgaacctgaagatgcgcgggcacttctgggtgggtgacgagctgtactgtgagaagcatgcccgccagcgctactccgcacctgccaccctcagctctcgggcctgagcccgccatgccctcagcctgcctcactgctgggccagggtcatgcctatataagttggcatggcagggacaatggtgggcagttgctcttacatgagctaagtttggagacctgaggcccctttgtcctcgctgggtgggccaaggtctgggacctgtcttggactgtgggagactcaccctcaccttgccaggcctctcccctgcaggactggcattgcactagtctgaggtggccactgcctttgatcaacctttgtgtgcgagggtctaagtagggtcgaacacagaagtgggaaggagaggggtgggccaggggctaatggtgtcactgtgtaaagtttttgacatactagctctataaa

SEQ ID NO: 487 - DNA, 531 bp, human
  misc_feature (78)..(78): n is a, c, g, or t
ccctatctgtactgctgtcttgcagctctgaacggtgcaacgtaatggcgacagaaagtatcttatttatatatagantatatatatgtaatttatataaaatatatagaaattattatatatatatattatacactctcatataatatatatatattcacacacatttgggttagaaaatctatggagacttcatcaatggtactatgttattagagaaatgctttaattttcatattccaatcagatggcatcttctatcccagctggttgggaagggattgaagatggtagcaagtgctgcactaagggcactcgcatttggtcattctcttgagagctaggaggggtcagcctgaggccggagaggaagggcttttgcctggggtgagagggtgagagacttgacctgaaagcagctgctgcccctgaggtgcagccagagtcctgtgtgcggacagagagaatggctaatgatnctccgcgtggaattgcctattgttttatgccacctgatgtgtctgcatgaga

SEQ ID NO: 488 - DNA, 149 bp, human
  misc_feature (55)..(55): n is a, c, g, or t
atattttgtatcatcgtgcctatagccgctgccaccgtgtataaatcctggtgtntgctccttatcctggacatgaatgtattgtacactgacgcgtccccactcctgtacagctgctttgtttctttgcaatgcattgtatggcttta

SEQ ID NO: 489 - DNA, 419 bp, human
ctgtgtgttctggaaatgcctctgggctgctgtgcaaaagcgcagcgtgcttgtgagtctcaggtggataactgtaggcagtcactaggcagtaagtggccgtggaggtggtgtctgctgtgcttaaagctccccctgcaccagcaaactcagggaaagctgcgtatttgtcttgcggttactggttaataacaactgatttcctcctgtcagacagcagaatctttctatcaggttagtgaaccttacggtttggaatctgtaatctgaatgtaacccagtttactttataaggtgtattcttaaattagtactccttgggaacagaattgccagacagccaactcagagtgactggtcaatggagaacactttgaacgagccatctttgcaatgccaaaatgtcctcccacaaggag

SEQ ID NO: 490 - DNA, 494 bp, human
caaggcatctgttgctttgggtcctccacgactcttaggcccgcctcaacaacccaggcacctcctaggtaggctcaaaggtagacccgtttccaccgcagcaggtgaacatgaccgtgttttcaactgtgtccacagttcagatccctttccagattgcaacctggcctgcatcccagctccttcctgctcgtgtcttaacctaagtgctttcttgtttgaaacgcctacaaacctccatgtggtagctcctttggcaaatgtcctgctgtggcgttttatgtgttgcttggagtctgtggggtcgtactccctcccctcccgtccccagggcagatttgattgaatgtttgctgaagttttgtctcttggtccacagtatttggaaaggtcactgaaaatgggtctttcagtcttggcatttcatttaggatctccatgagaaatgggcttcttgagccctgaaaatgtatattgtgtgtctcatctgtgaa

SEQ ID NO: 491 - DNA, 489 bp, human
acatgtccgtctgctggatcaacagaatcccgatgaggacttcagctgagtatagttcaacagttttgctgacagatgggaacaatcttttttttttttttttccaactgccatctatacaattttcttacagatgtcaaaagcagtctagtttatataagcattctgttacctgtgatattttttagactgaactgctccattcctagtcttaattaccatattcagggtacgaactggagggcttgtgtgttagcttctgaattggcaattggaggcggtagtggtcgtgcctgtgtgtatcagaagggataggtatcttgcctcctttctctcaggcagtgcaaatcaccctgtggaaaaccgatggacaggaaggagtgttacacactgcttaccctgatttattcagtggttttgttttcattctggaaccatactatcaaatggcgacagactgttccgttccacccccgtgaagagtaatca

SEQ ID NO: 492 - DNA, 444 bp, human
atctattccatgtgtgatttgcttgtagaaacaattttgaaagccccttgaggaaaataaaaatcaagaagaacacttttctcccttttccatacaaattaaaacttaacagcatcaaattattgggaccagaaaccaagtaatgtataatgtggcttttgttgagttaaataagatgctatataatggagaagaatttgaaaatgcacaaaaaaatcaatctacattatcagaacctgcagtgaaattaaacttatgttaaataaaaccagtttgcaggtgcacaaactatgagggtcttgtatccacgtaacacaggtagttacaaaaacatgttattgtactgtgtaaagatgcatagtcatctcatttggttggctttgtaccttgtaccttttttagccttggcttttgttgaactagaaccctcagcacatactgtgt

SEQ ID NO: 493 - DNA, 500 bp, human
tggagagtactttacagccattgtacagggtgactcactggaagagatttataacaaaatcaaacaaatcattgaggaccagtctgggcactacatttgggtcccatcccctgaaaaactctgaagaatcccctccaaccattctcttgtgaacagaagaaatcaagtccctcttccctcctccctcttcattcctgtccccatggggagaacaaatgctactgttcttgtccccttttttagatatgtcaaaaaaaattaagttttctagtcctgttcttttttttttttaagtttttgtttgtttcagtttattttttgggatgatgccatctcattcatcatgtgactgtgcccattcctgcatggacctttcccaagcgctagcataggtgcaaaatccatcagagccattgttttcataaaaaccaagcagaagtgaagagaaaagaggaggactgatggaaagacagactctggacagctgcacggcttgtgaa

SEQ ID NO: 494 - DNA, 403 bp, human
tgcacgccctgaagatgacctggcacgtgcactgctttacctgtgctgcctgcaagacgcccatccggaacagggccttctacatggaggagggcgtgccctattgcgagcgagactatgagaagatgtttggcacgaaatgccatggctgtgacttcaagatcgacgctggggaccgcttcctggaggccctgggcttcagctggcatgacacctgcttcgtctgtgcgatatgtcagatcaacctggaaggaaagaccttctactccaagaaggacaggcctctctgcaagagccatgccttctctcatgtgtgagccccttctgcccacagctgccgcggtggcccctagcctgaggggcctggagtcgtggccctgcatttctgggtagggctggcaat

SEQ ID NO: 495 - DNA, 490 bp, human
gtaggagcactacaccattggtttccaatactgcacacataagatacatacttgtgtgcagaaagtatcttcctccaggcttgtaatacccttcacatggaagattaatgagggaaatctttatattctgtataaaaacaaaagcaaatttatatactaaaatcatttgtctaaaaatttaagttgttttcaaataaaaattaaaatgcatttctgatatgcactgattgtgttgcctccagctttttttgctctctatgagtgactacttaagtcacttgttgagagggattatttactaattatatacttctcattcctgtaactccattccctttaaacagtggtgatatcaaatatacttccatccattgaatggggtatttttaacaacaacaaaagtgatatactaaaaaatgtattgcttaaggcttattgaatcattttgaagcactttgtgtatttgaaaactgctttataatctcattta

SEQ ID NO: 496 - DNA, 326 bp, human
cctcattccagcctttctatataaatagctctttcttgctgttttaatgtggtgcacactatagcctcacaaacctgttattccagtgtaatctgcagtgtcgtaactaaagttactggcttggtcttatttgcacagtttttgcgtcttgtttgcttcttgcatctgattaactagaatatttctctttcccccttttaatttgtgatgtcacttgaccccatttatgtgtaggagcactacaccattggtttccaatactgcacacataagatacatacttgtgtgcagaaagtatcttcctccaggcttgtaatacccttcac

SEQ ID NO: 497 - DNA, 410 bp, human
gattgttgaccgttaagaagaccttggatgttgttccagttatgctggattccacagtgaaatcatttaaaaccatctaaataaaccactatatattttatgaattacatgtggttttatatacacacacacacacacacacacacacacacacactctgacattttattacaagctgcatgtcctgaccctctttgaattaagtggactgtggcatgacattctgcaatactttgctgaattgaacactattgtgtcttaaatacttgcactaaatagtgcactgcaagaccagaaaattttacaatatttttctttacaatatgttctgtagtatgtttaccctctttatgaagtgaattaccatgctttgaataatgttcacttatacttcctgttcgaaattacga

SEQ ID NO: 498 - DNA, 444 bp, human
ctgacaaggagtgaagcgcccacccagcggatggacagcacccacctgggtttactcaagggtgtgcattcattttaggtgggatcgccacaggatttcatgttattttccttacggcttccttttcactgacctcatttgttgagttaatgtaagttaaatgtgtctatgatataactccactgtacatcatcctttgagtagtaaaggataaaagcatatatactacctatatgtatgtgctgtatgtgggcatttcattgagatctaattaatagctagcctatttatggttattcgttttagtaagttctgtgggagcaaggtatttaaaatcaaaactaataactacatcatggtttttgattaggatctaaatattcaggttttaagcctgctgcaaacttttaaaatattatgatagattctgtactacatgtggga

SEQ ID NO: 499 - DNA, 497 bp, human
agcagaggctctatttgacttcactggaaacagcaaactggagctgaatttcaaagctggagatgtgatcttcctcctcagtcggatcaacaaagactggctggagggcactgtccggggagccacgggcatcttccctctctccttcgtgaagatcctcaaagacttccctgaggaggacgaccccaccaactggctgcgttgctactactacgaagacaccatcagcaccatcaaggacatcgcggtggaggaagatctcagcagcactcccctattgaaagacctgctggagctcacaaggcgggagttccagagagaggacatagctctgaattaccgggacgctgagggggatctggttcggctgctgtcggatgaggacgtagcgctcatggtgcggcaggctcgtggcctcccctcccagaagcgcctcttcccctggaagctgcacatcacgcagaaggacaactacagggtctacaacacgatgccat

SEQ ID NO: 500 - DNA, 401 bp, human
ggagtcacaagacactgttgcagagaatgatgatggcgggttcagtgaggaatgggaagcccagagggacagtcatctagggcctcatcgctctacacctgagtcacgagctgctgtccaggaactttccagcagtatcctcgctggtgaagacccagaggaaaggggagtaaaacttggattgggagatttcattttctacagtgttctggttggtaaagcctcagcaacagccagtggagactggaacacaaccatagcctgtttcgtagccatattaattggtttgtgccttacattattactccttgccattttcaagaaagcattgccagctcttccaatctccatcacctttgggcttgttttctactttgccacagattatcttgtacagcctt

(19) United States
(12) Patent Application Publication
(10) Pub. No.: US 2008/0182246 A1
(43) Pub. Date: Jul. 31, 2008

(54) METHODS OF PREDICTING DISTANT METASTASIS OF LYMPH NODE-NEGATIVE PRIMARY BREAST CANCER USING BIOLOGICAL PATHWAY GENE EXPRESSION ANALYSIS

(76) Inventors: Yixin WANG, Basking Ridge, NJ (US); Yi ZHANG, San Diego, CA (US); Jack X. YU, San Diego, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/850,160

(22) Filed: Sep. 5, 2007

Related U.S. Application Data
(60) Provisional application No. 60/842,212, filed on Sep. 5, 2006.

(30) Foreign Application Priority Data
Sep. 5, 2007 (US) PCT/US07/77593

(51) Int. Cl. C12Q 1/68; C12Q 1/02

(52) U.S. Cl. 435/6; 435/29

(57) ABSTRACT

The present invention provides a method for predicting distant metastasis of lymph node negative primary breast cancer by obtaining breast cancer cells; isolating nucleic acid and/or protein from the cells; and analyzing the nucleic acid and/or protein to determine the presence, expression level or status of a Biomarker selected from the pathways in Table 2.

### STATEMENT REGARDING FEDERALLY SPONSORED RESEARCH OR DEVELOPMENT

No government funds were used to make this invention.

### REFERENCE TO SEQUENCE LISTING, OR A COMPUTER PROGRAM LISTING COMPACT DISK APPENDIX

Reference to a “Sequence Listing”, a table, or a computer program listing appendix submitted on a compact disc and an incorporation by reference of the material on the compact disc including duplicates and the files on each compact disc shall be specified.

### BACKGROUND OF THE INVENTION

Microarray technology has become a popular tool to classify breast cancer patients into subtypes, relapse and non-relapse, type of relapse, responder and non-responder³⁻¹¹. A concern for application of gene expression profiling is stability of the gene list as a signature¹². Considering that many genes have correlated expression on a chip, especially for genes involved in the same biological process, it is quite possible that different genes may be present in different signatures when different training sets of patients are used. Gene signatures to date for separating patients into different risk groups were derived based on the performance of individual genes, regardless of its biological processes or functions. It has been suggested that it might be more appropriate to interrogate the gene list for biological themes, rather than for individual genes^{1,2,8,13-19}.

### SUMMARY OF THE INVENTION

The present invention provides a method for predicting distant metastasis of lymph node negative primary breast cancer by obtaining breast cancer cells; isolating nucleic acid and/or protein from the cells; and analyzing the nucleic acid and/or protein to determine the presence, expression level or status of a Biomarker selected from the pathways in Table 2.

### BRIEF DESCRIPTION OF THE DRAWINGS

FIG. 1 Evaluation of the 500 gene signatures. Each of the 100-gene signatures for 80 randomly selected tumors in the training set was used to predict relapsed patients in the corresponding test set. Its performance was measured by the AUC of the ROC analysis. (a) Performance of the gene signatures for ER-positive patients in test sets. (b) Performance of the gene signatures for ER-negative patients in test sets. Distribution of AUC for the 500 prognostic signatures (left panels) as derived following the flow chart presented in FIG. 4. Distribution of AUC for the 500 random gene lists (right panels). To generate a gene list as a control, the clinic information for the ER-positive patients or ER-negative patients was permutated randomly and reassigned to the chip data.

FIG. 2 Association of the expression of individual genes with DMFS time for selected over-represented pathways. Geneplot function in the Global Test program^{1,2} was applied and the contribution of the individual genes in each selected pathway was plotted. The numbers at the X-axis represent the number of genes in the respective pathway in ER-positive or ER-negative tumors. The values at the Y-axis, represent the contribution (influence) of each individual gene in the selected pathway with DMFS. Negative values indicate there is no association between the gene expression and DMFS. Each thin horizontal line in a bar (influence) indicates one standard deviation away from the reference point, two or more horizontal lines in a bar indicates that the association of the corresponding gene with DMFS is statistically significant. The green bars reflect genes that are positively associated with DMFS, indicating a higher expression in tumors without metastatic capability. The red bars reflect genes that are negatively associated with DMFS, indicative of higher expression in tumors with metastatic capability. (a) Apoptosis pathway consisting of 282 genes in ER-positive tumors. (b) Regulation of cell growth pathway consisting of 58 genes in ER-negative tumors. (c) Regulation of cell cycle pathway consisting of 228 genes in ER-positive tumors. (d) Cell adhesion pathway consisting of 327 genes in ER-negative tumors. (e) Immune response pathway consisting of 379 genes in ER-positive tumors. (f) Regulation of G-coupled receptor signaling pathway consisting of 20 genes in ER-negative tumors. (g) Mitosis pathway consisting of 100 genes in ER-positive tumors. (h) Skeletal development pathway consisting of 105 genes in ER-negative tumors.

FIG. 3 Validation of pathway-based breast cancer classifiers constructed from the optimal significant genes of the two most significant pathways for both ER-positive and ER-negative tumors. A recently published data set for which samples were hybridized on Affymetrix U133A chip²¹, including 189 invasive breast carcinomas with survival information, was used. Among them, 153 tumors were from lymph node negative patients. After removing one patient who died 15 days after surgery, the remaining 152 patients were used to validate the signatures. The 152 patients set consisted of 125 ER-positive tumors and 27 ER-negative tumors based on the expression level of ER gene on the chip. (a) Receiver operating characteristic (ROC) analysis of the 38-gene signature for ER-positive tumors. (b) Kaplan-Meier analysis of patients with ER-positive tumors as a function of the 38-gene signature. The DMFS probabilities (and their 95% confidence intervals) at 60 and 120 months, respectively, were 92.7% (86.0% to 99.9%), or 74.5% (62.0% to 89.5%) for the good signature curve, 59.9%% (49.0% to 73.2%), or 48.5% (36.8% to 63.9%) for the poor signature curve. (c) ROC analysis of the 12-gene signature for ER-negative tumors. (d) Kaplan-Meier analysis of patients with ER-negative tumors as function of the 12-gene signature. The DMFS probabilities (and their 95% confidence intervals) at 60 and 120 months, respectively, were both 94.1% (83.6% to 100%) for the good signature curve, and 40.0% (18.7% to 85.5%), or 26.7% (8.9% to 80.3%) for the poor signature curve. (e) ROC analysis of a combined 50-gene signatures for ER-positive and ER-negative tumors. (f) Kaplan-Meier analysis of 152 breast cancer patients as a function of the 50-gene signature. The DMFS probabilities (and their 95% confidence intervals) at 60 and 120 months, respectively, were 93.0% (87.3% to 99.1%), or 79.3% (69.2% to 91.0%) for the good signature curve, and 57.2% (46.9% to 69.7%), or 45.4% (34.6% to 59.7%) for the poor signature curve.

FIG. 4 shows a work flow of data analysis.

FIG. 5 shows top 20 prognostic pathways in ER-positive tumors obtained from Association of the expression of individual genes with DMFS time for selected over-represented pathways. Geneplot function in the Global Test program^{1,2} was applied and the contribution of the individual genes in each selected pathway is plotted. The numbers at the X-axis represent the number of genes in the respective pathway in ER-positive tumors. The values at the Y-axis, represent the contribution (influence) of each individual gene in the selected pathway with DMFS. Negative values indicate there is no association between the gene expression and DMFS. Each thin horizontal line in a bar (influence) indicates one standard deviation away from the reference point, two or more horizontal lines in a bar indicates that the association of the corresponding gene with DMFS is statistically significant. The green bars reflect genes that are positively associated with DMFS, indicating a higher expression in tumors without metastatic capability. The red bars reflect genes that are negatively associated with DMFS, indicative of higher expression in tumors with metastatic capability.

### DETAILED DESCRIPTION

The present invention provides a method for predicting distant metastasis of lymph node negative primary breast cancer by obtaining breast cancer cells; isolating nucleic acid and/or protein from the cells; and analyzing the nucleic acid and/or protein to determine the presence, expression level or status of a Biomarker selected from the pathways in Table 2.

A Biomarker is any indicia of an indicated Marker nucleic acid/protein. Nucleic acids can be any known in the art including, without limitation, nuclear, mitochondrial (homeoplasmy, heteroplasmy), viral, bacterial, fungal, mycoplasmal, etc. The indicia can be direct or indirect and measure over- or under-expression of the gene given the physiologic parameters and in comparison to an internal control, placebo, normal tissue or another carcinoma. Biomarkers include, without limitation, nucleic acids and proteins (both over and under-expression and direct and indirect). Using nucleic acids as Biomarkers can include any method known in the art including, without limitation, measuring DNA amplification, deletion, insertion, duplication, RNA, micro RNA (miRNA), loss of heterozygosity (LOH), single nucleotide polymorphisms (SNPs, Brookes (1999)), copy number polymorphisms (CNPs) either directly or upon genome amplification, microsatellite DNA, epigenetic changes such as DNA hypo- or hyper-methylation and FISH. Using proteins as Biomarkers includes any method known in the art including, without limitation, measuring amount, activity, modifications such as glycosylation, phosphorylation, ADP-ribosylation, ubiquitination, etc., or immunohistochemistry (IHC) and turnover. Other Biomarkers include imaging, molecular profiling, cell count and apoptosis Markers.

“Origin” as referred to in ‘tissue of origin’ means either the tissue type (lung, colon, etc.) or the histological type (adenocarcinoma, squamous cell carcinoma, etc.) depending on the particular medical circumstances and will be understood by anyone of skill in the art.

A Marker gene corresponds to the sequence designated by a SEQ ID NO when it contains that sequence. A gene segment or fragment corresponds to the sequence of such gene when it contains a portion of the referenced sequence or its complement sufficient to distinguish it as being the sequence of the gene. A gene expression product corresponds to such sequence when its RNA, mRNA, or cDNA hybridizes to the composition having such sequence (e.g. a probe) or, in the case of a peptide or protein, it is encoded by such mRNA. A segment or fragment of a gene expression product corresponds to the sequence of such gene or gene expression product when it contains a portion of the referenced gene expression product or its complement sufficient to distinguish it as being the sequence of the gene or gene expression product.

The inventive methods, compositions, articles, and kits of described and claimed in this specification include one or more Marker genes. “Marker” or “Marker gene” is used throughout this specification to refer to genes and gene expression products that correspond with any gene the over- or under-expression of which is associated with an indication or tissue type.

Preferred methods for establishing gene expression profiles include determining the amount of RNA that is produced by a gene that can code for a protein or peptide. This is accomplished by reverse transcriptase PCR (RT-PCR), competitive RT-PCR, real time RT-PCR, differential display RT-PCR, Northern Blot analysis and other related tests. While it is possible to conduct these techniques using individual PCR reactions, it is best to amplify complementary DNA (cDNA) or complementary RNA (cRNA) produced from mRNA and analyze it via microarray. A number of different array configurations and methods for their production are known to those of skill in the art and are described in for instance, U.S. Pat. Nos. 5,445,934; 5,532,128; 5,556,752; 5,242,974; 5,384,261; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,599,695; 5,624,711; 5,658,734; and 5,700,637.

Microarray technology allows for the measurement of the steady-state mRNA level of thousands of genes simultaneously thereby presenting a powerful tool for identifying effects such as the onset, arrest, or modulation of uncontrolled cell proliferation. Two microarray technologies are currently in wide use. The first are cDNA arrays and the second are oligonucleotide arrays. Although differences exist in the construction of these chips, essentially all downstream data analysis and output are the same. The product of these analyses are typically measurements of the intensity of the signal received from a labeled probe used to detect a cDNA sequence from the sample that hybridizes to a nucleic acid sequence at a known location on the microarray. Typically, the intensity of the signal is proportional to the quantity of cDNA, and thus mRNA, expressed in the sample cells. A large number of such techniques are available and useful. Preferred methods for determining gene expression can be found in U.S. Pat. Nos. 6,271,002; 6,218,122; 6,218,114; and 6,004,755.

Analysis of the expression levels is conducted by comparing such signal intensities. This is best done by generating a ratio matrix of the expression intensities of genes in a test sample versus those in a control sample. For instance, the gene expression intensities from a diseased tissue can be compared with the expression intensities generated from benign or normal tissue of the same type. A ratio of these expression intensities indicates the fold-change in gene expression between the test and control samples.

The selection can be based on statistical tests that produce ranked lists related to the evidence of significance for each gene's differential expression between factors related to the tumor's original site of origin. Examples of such tests include ANOVA and Kruskal-Wallis. The rankings can be used as weightings in a model designed to interpret the summation of such weights, up to a cutoff, as the preponderance of evidence in favor of one class over another. Previous evidence as described in the literature may also be used to adjust the weightings.

A preferred embodiment is to normalize each measurement by identifying a stable control set and scaling this set to zero variance across all samples. This control set is defined as any single endogenous transcript or set of endogenous transcripts affected by systematic error in the assay, and not known to change independently of this error. All Markers are adjusted by the sample specific factor that generates zero variance for any descriptive statistic of the control set, such as mean or median, or for a direct measurement. Alternatively, if the premise of variation of controls related only to systematic error is not true, yet the resulting classification error is less when normalization is performed, the control set will still be used as stated. Non-endogenous spike controls could also be helpful, but are not preferred.

Gene expression profiles can be displayed in a number of ways. The most common is to arrange raw fluorescence intensities or ratio matrix into a graphical dendogram where columns indicate test samples and rows indicate genes. The data are arranged so genes that have similar expression profiles are proximal to each other. The expression ratio for each gene is visualized as a color. For example, a ratio less than one (down-regulation) appears in the blue portion of the spectrum while a ratio greater than one (up-regulation) appears in the red portion of the spectrum. Commercially available computer software programs are available to display such data including “Genespring” (Silicon Genetics, Inc.) and “Discovery” and “Infer” (Partek, Inc.)

In the case of measuring protein levels to determine gene expression, any method known in the art is suitable provided it results in adequate specificity and sensitivity. For example, protein levels can be measured by binding to an antibody or antibody fragment specific for the protein and measuring the amount of antibody-bound protein. Antibodies can be labeled by radioactive, fluorescent or other detectable reagents to facilitate detection. Methods of detection include, without limitation, enzyme-linked immunosorbent assay (ELISA) and immunoblot techniques.

Modulated genes used in the methods of the invention are described in the Examples. The genes that are differentially expressed are either up regulated or down regulated in patients with carcinoma of a particular origin relative to those with carcinomas from different origins. Up regulation and down regulation are relative terms meaning that a detectable difference (beyond the contribution of noise in the system used to measure it) is found in the amount of expression of the genes relative to some baseline. In this case, the baseline is determined based on the algorithm. The genes of interest in the diseased cells are then either up regulated or down regulated relative to the baseline level using the same measurement method. Diseased, in this context, refers to an alteration of the state of a body that interrupts or disturbs, or has the potential to disturb, proper performance of bodily functions as occurs with the uncontrolled proliferation of cells. Someone is diagnosed with a disease when some aspect of that person's genotype or phenotype is consistent with the presence of the disease. However, the act of conducting a diagnosis or prognosis may include the determination of disease/status issues such as determining the likelihood of relapse, type of therapy and therapy monitoring. In therapy monitoring, clinical judgments are made regarding the effect of a given course of therapy by comparing the expression of genes over time to determine whether the gene expression profiles have changed or are changing to patterns more consistent with normal tissue.

Genes can be grouped so that information obtained about the set of genes in the group provides a sound basis for making a clinically relevant judgment such as a diagnosis, prognosis, or treatment choice. These sets of genes make up the portfolios of the invention. As with most diagnostic Markers, it is often desirable to use the fewest number of Markers sufficient to make a correct medical judgment. This prevents a delay in treatment pending further analysis as well unproductive use of time and resources.

One method of establishing gene expression portfolios is through the use of optimization algorithms such as the mean variance algorithm widely used in establishing stock portfolios. This method is described in detail in 20030194734. Essentially, the method calls for the establishment of a set of inputs (stocks in financial applications, expression as measured by intensity here) that will optimize the return (e.g., signal that is generated) one receives for using it while minimizing the variability of the return. Many commercial software programs are available to conduct such operations. “Wagner Associates Mean-Variance Optimization Application,” referred to as “Wagner Software” throughout this specification, is preferred. This software uses functions from the “Wagner Associates Mean-Variance Optimization Library” to determine an efficient frontier and optimal portfolios in the Markowitz sense is preferred. Markowitz (1952). Use of this type of software requires that microarray data be transformed so that it can be treated as an input in the way stock return and risk measurements are used when the software is used for its intended financial analysis purposes.

The process of selecting a portfolio can also include the application of heuristic rules. Preferably, such rules are formulated based on biology and an understanding of the technology used to produce clinical results. More preferably, they are applied to output from the optimization method. For example, the mean variance method of portfolio selection can be applied to microarray data for a number of genes differentially expressed in subjects with cancer. Output from the method would be an optimized set of genes that could include some genes that are expressed in peripheral blood as well as in diseased tissue. If samples used in the testing method are obtained from peripheral blood and certain genes differentially expressed in instances of cancer could also be differentially expressed in peripheral blood, then a heuristic rule can be applied in which a portfolio is selected from the efficient frontier excluding those that are differentially expressed in peripheral blood. Of course, the rule can be applied prior to the formation of the efficient frontier by, for example, applying the rule during data pre-selection.

Other heuristic rules can be applied that are not necessarily related to the biology in question. For example, one can apply a rule that only a prescribed percentage of the portfolio can be represented by a particular gene or group of genes. Commercially available software such as the Wagner Software readily accommodates these types of heuristics. This can be useful, for example, when factors other than accuracy and precision (e.g., anticipated licensing fees) have an impact on the desirability of including one or more genes.

The gene expression profiles of this invention can also be used in conjunction with other non-genetic diagnostic methods useful in cancer diagnosis, prognosis, or treatment monitoring. For example, in some circumstances it is beneficial to combine the diagnostic power of the gene expression based methods described above with data from conventional Markers such as serum protein Markers (e.g., Cancer Antigen 27.29 (“CA 27.29”)). A range of such Markers exists including such analytes as CA 27.29. In one such method, blood is periodically taken from a treated patient and then subjected to an enzyme immunoassay for one of the serum Markers described above. When the concentration of the Marker suggests the return of tumors or failure of therapy, a sample source amenable to gene expression analysis is taken. Where a suspicious mass exists, a fine needle aspirate (FNA) is taken and gene expression profiles of cells taken from the mass are then analyzed as described above. Alternatively, tissue samples may be taken from areas adjacent to the tissue from which a tumor was previously removed. This approach can be particularly useful when other testing produces ambiguous results.

The present invention provides a method for analyzing a biological specimen for the presence of cells specific for an indication by: a) enriching cells from the specimen; b) isolating nucleic acid and/or protein from the cells; and c) analyzing the nucleic acid and/or protein to determine the presence, expression level or status of a Biomarker specific for the indication.

The biological specimen can be any known in the art including, without limitation, urine, blood, serum, plasma, lymph, sputum, semen, saliva, tears, pleural fluid, pulmonary fluid, bronchial lavage, synovial fluid, peritoneal fluid, ascites, amniotic fluid, bone marrow, bone marrow aspirate, cerebrospinal fluid, tissue lysate or homogenate or a cell pellet. See, e.g. 20030219842.

The indication can include any known in the art including, without limitation, cancer, risk assessment of inherited genetic pre-disposition, identification of tissue of origin of a cancer cell such as a CTC 60/887,625, identifying mutations in hereditary diseases, disease status (staging), prognosis, diagnosis, monitoring, response to treatment, choice of treatment (pharmacologic), infection (viral, bacterial, mycoplasmal, fungal), chemosensitivity U.S. Pat. No. 7,112,415, drug sensitivity, metastatic potential or identifying mutations in hereditary diseases.

Cells enrichment can be by any method known in the art including, without limitation, by antibody/magnetic separation, (Immunicon, Miltenyi, Dynal) U.S. Pat. No. 6,602,422, 5,200,048, fluorescence activated cell sorting, (FACs) U.S. Pat. No. 7,018,804, filtration or manually. The manual enrichment can be for instance by prostate massage. Goessl et al. (2001) Urol 58:335-338.

The nucleic acid can be any known in the art including, without limitation, is nuclear, mitochondrial (homeoplasmy, heteroplasmy), viral, bacterial, fungal or mycoplasmal.

Methods of isolating nucleic acid and protein are well known in the art. See e.g. U.S. Pat. No. 6,992,182, RNA www.aibion.com/techlib/basics/rnaisol/index.htlm, and 20070054287.

DNA analysis can be any known in the art including, without limitation, methylation, de-methylation, karyotyping, ploidy (aneuploidy, polyploidy), DNA integrity (assessed through gels or spectrophotometry), translocations, mutations, gene fusions, activation—de-activation, single nucleotide polymorphisms (SNPs), copy number or whole genome amplification to detect genetic makeup. RNA analysis includes any known in the art including, without limitation, q-RT-PCR, miRNA or post-transcription modifications. Protein analysis includes any known in the art including, without limitation, antibody detection, post-translation modifications or turnover. The proteins can be cell surface markers, preferably epithelial, endothelial, viral or cell type. The Biomarker can be related to viral/bacterial infection, insult or antigen expression.

The claimed invention can be used for instance to determine metastatic potential of a cell from a biological specimen by isolating nucleic acid and/or protein from the cells; and analyzing the nucleic acid and/or protein to determine the presence, expression level or status of a Biomarker specific for metastatic potential.

The cells of the claimed invention can be used for instance to identify mutations in hereditary diseases cell from a biological specimen by isolating nucleic acid and/or protein from the cells; and analyzing the nucleic acid and/or protein to determine the presence, expression level or status of a Biomarker specific for specific for a hereditary disease.

The cells of the claimed invention can be used for instance to obtain and preserve cellular material and constituent parts thereof such as nucleic acid and/or protein. The constituent parts can be used for instance to make tumor cell vaccines or in immune cell therapy. 20060093612, 20050249711.

Kits made according to the invention include formatted assays for determining the gene expression profiles. These can include all or some of the materials needed to conduct the assays such as reagents and instructions and a medium through which Biomarkers are assayed.

Articles of this invention include representations of the gene expression profiles useful for treating, diagnosing, prognosticating, and otherwise assessing diseases. These profile representations are reduced to a medium that can be automatically read by a machine such as computer readable media (magnetic, optical, and the like). The articles can also include instructions for assessing the gene expression profiles in such media. For example, the articles may comprise a CD ROM having computer instructions for comparing gene expression profiles of the portfolios of genes described above. The articles may also have gene expression profiles digitally recorded therein so that they may be compared with gene expression data from patient samples. Alternatively, the profiles can be recorded in different representational format. A graphical recordation is one such format. Clustering algorithms such as those incorporated in “DISCOVERY” and “INFER” software from Partek, Inc. mentioned above can best assist in the visualization of such data.

Different types of articles of manufacture according to the invention are media or formatted assays used to reveal gene expression profiles. These can comprise, for example, microarrays in which sequence complements or probes are affixed to a matrix to which the sequences indicative of the genes of interest combine creating a readable determinant of their presence. Alternatively, articles according to the invention can be fashioned into reagent kits for conducting hybridization, amplification, and signal generation indicative of the level of expression of the genes of interest for detecting cancer.

The present invention defines specific marker portfolios that have been characterized to detect a single circulating breast tumor cell in a background of peripheral blood. The molecular characterization multiplex assay portfolio has been optimized for use as a QRT-PCR multiplex assay where the molecular characterization multiplex contains 2 tissue of origin markers, 1 epithelial marker and a housekeeping marker. QRT-PCR will be carried out on the Smartcycler II for the molecular characterization multiplex assay. The molecular characterization singlex assay portfolio has been optimized for use as a QRT-PCR assay where each marker is run in a single reaction that utilizes 3 cancer status markers, 1 epithelial marker and a housekeeping marker. Unlike the RPA multiplex assay the molecular characterization singlex assay will be run on the Applied Biosystems (ABI) 7900HT and will use a 384 well plate as it platform. The molecular characterization multiplex assay and singlex assay portfolios accurately detect a single circulating epithelial cell enabling the clinician to predict recurrence. The molecular characterization multiplex assay utilizes Thermus thermophilus (TTH) DNA polymerase due to its ability to carry out both reverse transcriptase and polymerase chain reaction in a single reaction. In contrast, the molecular characterization singlex assay utilizes the Applied Biosystems One-Step Master Mix which is a two enzyme reaction incorporating MMLV for reverse transcription and Taq polymerase for PCR. Assay designs are specific to RNA by the incorporation of an exon-intron junction so that genomic DNA is not efficiently amplified and detected.

Knowledge of biological processes may be more relevant for understanding of the disease than information on differentially expressed genes. We have investigated distinct biological pathways associated with the metastatic capability of lymph-node negative primary breast tumors. A re-sampling method was used to create 500 different training sets, and to derive the corresponding gene signatures for estrogen receptor (ER)-positive and -negative tumors. The constructed gene signatures were mapped to Gene Ontology Biological Process (GOBP) to identify over-represented pathways related to patient outcomes. Global Test program^{1,2} was used to confirm that these biological pathways were associated with the development of metastases. Furthermore, by mapping 4 published prognostic gene signatures with more than 60 genes to the top 20 pathways, each of them can be mapped to 19 of the top distinct pathways despite a minimum overlap of identical genes. Our study provides a new way to understand the mechanisms of breast cancer progression and to derive a pathway-based signatures for prognosis.

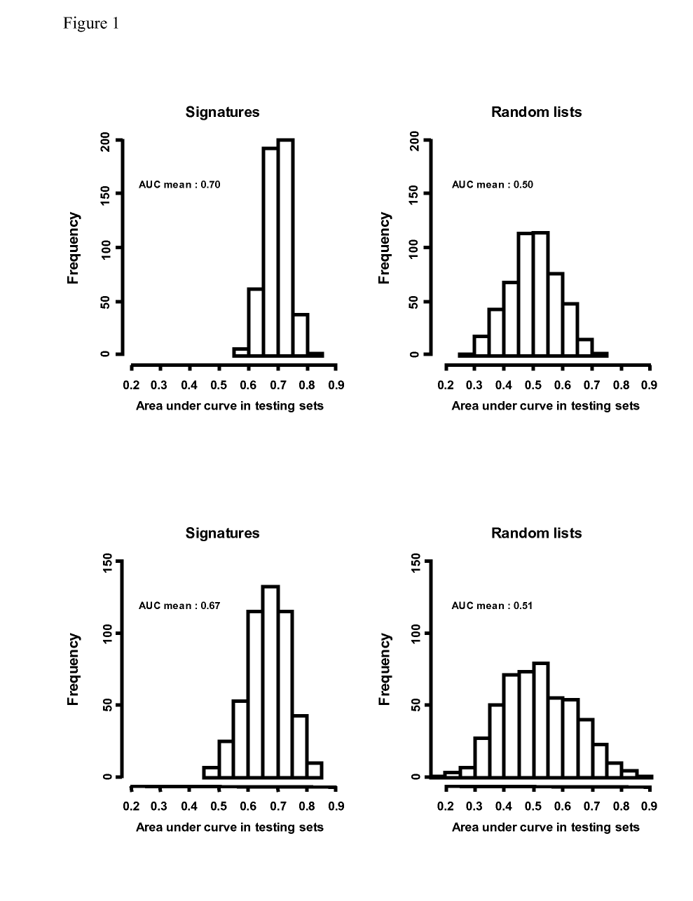

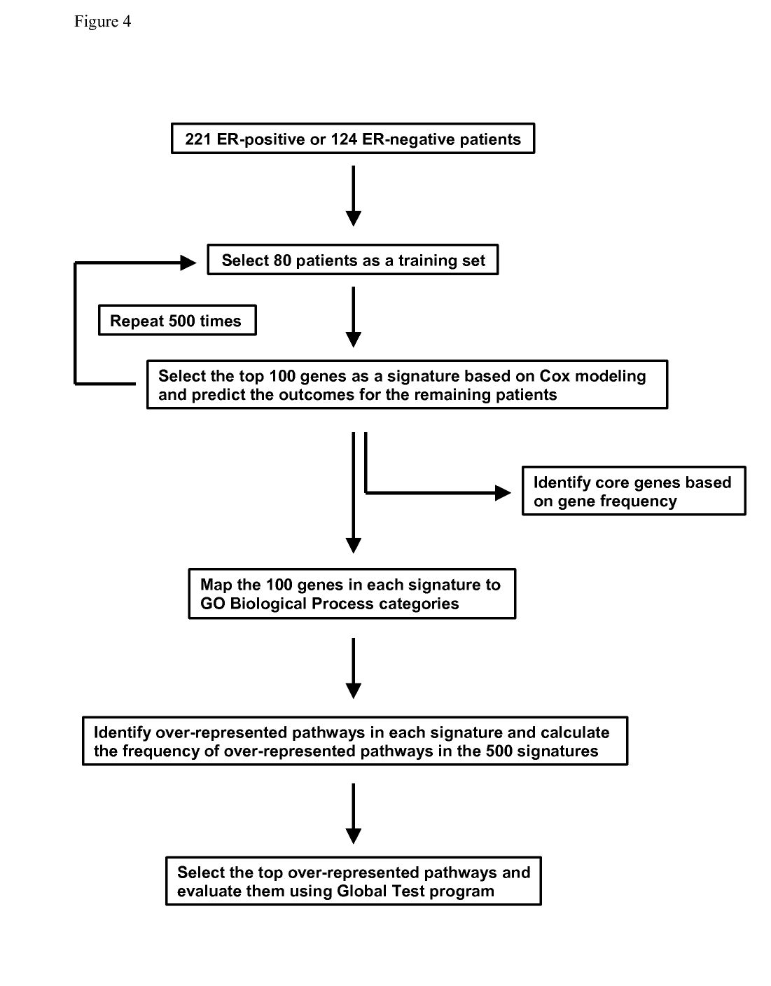

We investigated the various prognostic gene signatures derived from different patient groups with an aim towards understanding the underlying biological pathways. Since gene expression patterns of ER-subgroups of breast tumors are quite different^{3-6,8,20}, data analysis to derive gene signatures and subsequent pathway analysis was conducted separately⁸. For either ER-positive or ER-negative patients, 80 samples were randomly selected as a training set and the top 100 genes were used as a signature to predict tumor recurrence for the remaining ER-positive or ER-negative patients (FIG. 4). The area under curve (AUC) of receiver operating characteristic (ROC) analysis with distant metastasis within 5 years as a defining point was used as a measurement of the performance of a signature in a corresponding test set. The above procedure was repeated 500 times. The average of AUCs for the 500 signatures in the test sets was 0.70 whereas the average of AUCs for the 500 control gene lists was 0.50, indicating random prediction (FIG. 1a). For ER-negative datasets, these values were 0.67 and 0.51, respectively (FIG. 1b). Multiple gene signatures could be identified with similar performance while the genes in individual signatures can be substituted. The top 20 genes ranked by their frequency in the 500 signatures for ER-positive or ER-negative tumors are shown in Table 1. The most frequently present genes were those for KIAA0241 protein (KIAA0241) for ER-positive tumors, and zinc finger protein multitype 2 (ZFPM2) for ER-negative tumors, respectively, while there was no overlap between genes of the two core gene lists. For Sequence ID Numbers see the sequence listing table.

TABLE 1
Genes with highest frequencies in 500 signatures

| Gene title | Gene symbol | Frequency |
|---|---|---|
| Top 20 core genes from ER-positive tumors | | |
| KIAA0241 protein | KIAA0241 | 321 |
| CD44 antigen (homing function and Indian blood group system) | CD44 | 286 |
| ATP-binding cassette, sub-family C (CFTR/MRP), member 5 | ABCC5 | 251 |
| serine/threonine kinase 6 | STK6 | 245 |
| cytochrome c, somatic | CYCS | 235 |
| KIAA0406 gene product | KIA0406 | 212 |
| uridine-cytidine kinase 1-like 1 | UCKL1 | 201 |
| zinc finger, CCHC domain containing 8 | ZCCHC8 | 188 |
| Rac GTPase activating protein 1 | RACGAP1 | 186 |
| staufen, RNA binding protein (Drosophila) | STAU | 176 |
| lactamase, beta 2 | LACTB2 | 175 |
| eukaryotic translation elongation factor 1 alpha 2 | EEF1A2 | 172 |
| RAE1 RNA export 1 homolog (S. pombe) | RAE1 | 153 |
| tuftelin 1 | TUFT1 | 150 |
| zinc finger protein 36, C3H type-like 2 | ZFP36L2 | 150 |
| origin recognition complex, subunit 6 homolog-like (yeast) | ORC6L | 143 |
| zinc finger protein 623 | ZNF623 | 140 |
| extra spindle poles like 1 | ESPL1 | 139 |
| transcription elongation factor B (SIII), polypeptide 1 | TCEB1 | 138 |
| ribosomal protein S6 kinase, 70 kDa, polypeptide 1 | RPS6KB1 | 127 |
| Top 20 core genes from ER-negative tumors | | |
| zinc finger protein, multitype 2 | ZFPM2 | 445 |
| ribosomal protein L26-like 1 | RPL26L1 | 372 |
| hypothetical protein FLJ14346 | FLJ14346 | 372 |
| mitogen-activated protein kinase-activated protein kinase 2 | MAPKAPK2 | 347 |
| collagen, type II, alpha 1 | COL2A1 | 340 |
| muscleblind-like 2 (Drosophila) | MBNL2 | 320 |
| G protein-coupled receptor 124 | GPR124 | 314 |
| splicing factor, arginine/serine-rich 11 | SFRS11 | 300 |
| heterogeneous nuclear ribonucleoprotein A1 | HNRPA1 | 297 |
| CDC42 binding protein kinase alpha (DMPK-like) | CDC42BPA | 296 |
| regulator of G-protein signalling 4 | RGS4 | 276 |
| transient receptor potential cation channel, subfamily C, member 1 | TRPC1 | 265 |
| transcription factor 8 (represses interleukin 2 expression) | TCF8 | 263 |
| chromosome 6 open reading frame 210 | C6orf210 | 262 |
| dynamin 3 | DNM3 | 260 |
| centrosome protein Cep63 | Cep63 | 251 |
| tumor necrosis factor (ligand) superfamily, member 13 | TNFSF13 | 251 |
| dapper, antagonist of beta-catenin, homolog 1 (Xenopus laevis) | DACT1 | 248 |
| heterogeneous nuclear ribonucleoprotein A1 | HNRPA1 | 245 |
| reversion-inducing-cysteine-rich protein with kazal motifs | RECK | 243 |

In Table 1, the top 20 genes are ranked by their frequency in the 500 signatures of 100 genes for ER-positive and ER-negative tumors (for details see FIG. 4).

The biological pathways are distinct for ER-positive and -negative tumors. For ER-positive tumors, many pathways that are related with cell division are present in the top 20 over-represented pathways, in addition to a couple of immune-related pathways (Table 4).

TABLE 4
Top 20 pathways over-represented in the 500 signatures and evaluation by
Global Test program

| Pathways for ER+ tumors | | | Pathways for ER− tumors | | |
|---|---|---|---|---|---|
| GO_Process | GO_ID | Frequency | GO_Process | GO_ID | Frequency |
| mitosis | 7067 | 256 | nuclear mRNA splicing, via spliceosome | 398 | 203 |
| apoptosis | 6915 | 250 | RNA splicing | 8380 | 192 |
| oncogenesis | 7084 | 228 | protein complex assembly | 6461 | 183 |
| regulation of cell cycle | 74 | 203 | endocytosis | 6897 | 166 |
| cell surface recepter-linked signal | 7166 | 172 | skeletal development | 1501 | 160 |
| transduction | | | | | |
| immune response | 6955 | 167 | cation transport | 6812 | 160 |
| cytokinesis | 910 | 165 | signal transduction | 7165 | 160 |
| ubiquitin-dependent protein catabolism | 6511 | 158 | regulation of G-protein coupled receptor signaling | 8277 | 153 |
| DNA repair | 6281 | 156 | protein amino acid phosphorylation | 6468 | 151 |
| protein biosynthesis | 6412 | 145 | regulation of cell growth | 1558 | 136 |
| intracellular protein transport | 6886 | 141 | intracellular signaling cascade | 7242 | 135 |
| cell cycle | 7049 | 138 | protein modification | 6464 | 132 |
| cellular defense response | 6968 | 131 | cell adhesion | 7155 | 110 |
| induction of apoptosis | 6917 | 115 | regulation of transcription from Pol II promoter | 6357 | 109 |
| protein amino acid phosphorylation | 6468 | 114 | protein biosynthesis | 6412 | 99 |
| mitotic chromosome segregation | 70 | 98 | calcium ion transport | 6816 | 93 |
| cell motility | 6928 | 93 | regulation of cell cycle | 74 | 88 |
| DNA replication | 6260 | 92 | carbohydrate metabolism | 5975 | 86 |
| chemotaxis | 6935 | 89 | mRNA processing | 6397 | 81 |
| metabolism | 8152 | 83 | cell cycle | 7049 | 72 |

All of the 20 pathways had a significant association with distant metastasis-free survival (DMFS) by Global Testing program. The top 2 most significant being Apoptosis, and Regulation of cell cycle (Table 2). For ER-negative tumors, many of the top 20 pathways are related with RNA processing, transportation and signal transduction (Table 4). Eighteen of the top 20 pathways demonstrated significant association with DMFS, the 2 most significant being Regulation of cell growth, and Regulation of G-protein coupled receptor signaling (Table 2).

TABLE 2
Top 20 pathways in the 500 signatures of ER-positive
and ER-negative tumors evaluated by Global Test

| Pathways | GO_ID | P | Frequency |
|---|---|---|---|
| ER-positive tumors | | | |
| Apoptosis | 6915 | 3.06E−7 | 250 |
| Regulation of cell cycle | 74 | 2.46E−5 | 203 |
| Protein amino acid | 6468 | 2.48E−5 | 114 |
| phosphorylation | | | |
| Cytokinesis | 910 | 6.13E−5 | 165 |
| Cell motility | 6928 | 0.00015 | 93 |
| Cell cycle | 7049 | 0.00028 | 138 |

In Table 2, each of the top 20 over-represented pathways that have the highest frequencies in the 500 signatures of ER-positive and ER-negative tumors (see Table 5) were subjected to Global Test program^{1,2}. The Global Test examines the association of a group of genes as a whole to a specific clinical parameter, in this case DMFS, and generates an asymptotic theory P value for the pathway^{1,2}. The pathways are ranked by their P value in the respective ER-subgroup of tumors.

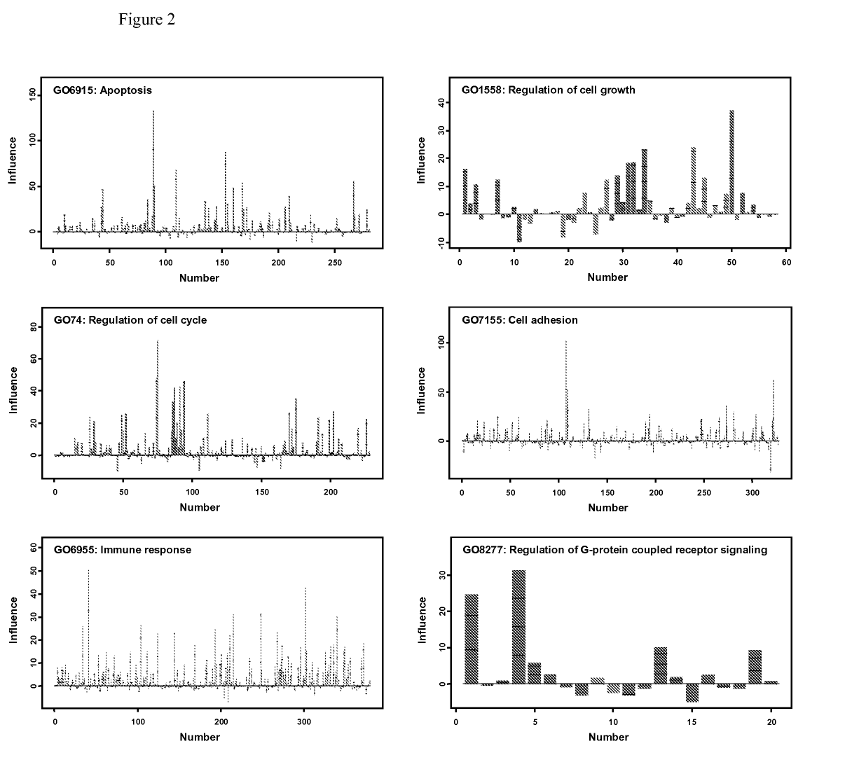

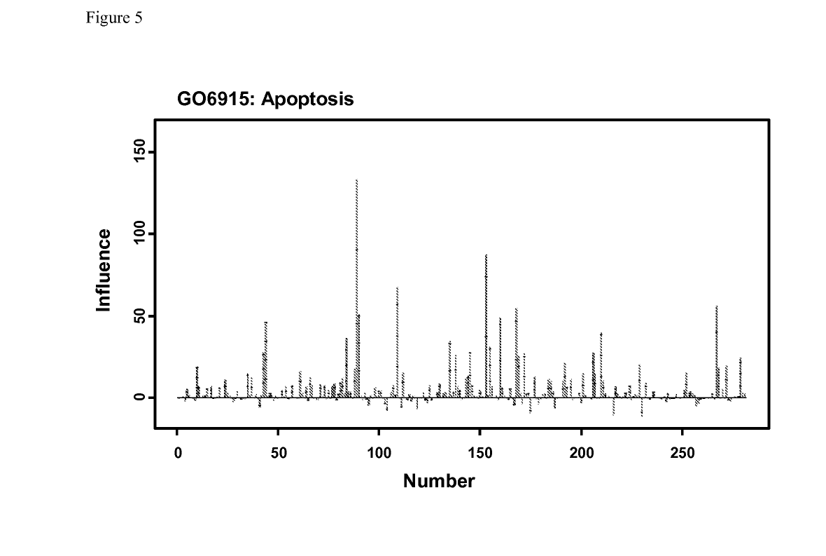

The contribution of individual genes in the top over-represented pathways to the association with DMFS, and their significance, were determined for ER-positive (FIG. 5, and Table 5 online) and ER-negative tumors (FIG. 6 online, and Table 6). In these pathways, multiple genes are positively associated with DMFS, indicating a higher expression in tumors without metastatic capability, while other genes show a negative association, indicative of a higher expression in metastatic tumors. In ER-positive tumors such pathways with a mixed association included the top 2 significant pathways Apoptosis (FIG. 2a) and Regulation of cell cycle (FIG. 2c). There were also a number of pathways that had dominant positive or negative correlation with DMFS. For example, Immune response of GOBP contains 379 probe sets, of which most showed positive correlation to DMFS (FIG. 2e). Similarly in Cellular defense response and Chemotaxis, most genes displayed a strong positive correlation with DMFS (FIG. 5 online). On the other hand, genes in Mitosis (FIG. 2g), Mitotic chromosome segregation, and Cell cycle, showed a dominant negative correlation with DMFS (FIG. 5). Thus, in general the cell division-related pathways have dominant negative correlation with survival time, while immune-related pathways have dominant positive correlation. This indicates that ER-positive tumors with metastatic capability tend to have higher cell division rates and induce lower immune activities from the host body.

TABLE 5
Significant genes in the top 20 pathways for ER-positive tumors

| | | | | | | |
|---|---|---|---|---|---|---|
| | | | | | Gene | |
| PSID | influence | sd | z-score | info | Symbol | Gene Title |

Apoptosis

| | | | | | | |
|---|---|---|---|---|---|---|
| 208905_at | 13.03 | 3.04 | 4.29 | − | CYCS | cytochrome c, somatic |
| 202731_at | 46.15 | 11.50 | 4.01 | + | PDCD4 | programmed cell death 4 |
| 204817_at | 36.39 | 9.77 | 3.73 | − | ESPL1 | extra spindle poles like 1 |
| 206150_at | 67.60 | 18.92 | 3.57 | + | TNFRSF7 | tumor necrosis factor receptor superfamily, |
| | | | | | | member 7 |
| 38158_at | 24.65 | 7.23 | 3.41 | − | ESPL1 | extra spindle poles like 1 |
| 202730_s_at | 27.75 | 8.73 | 3.18 | + | PDCD4 | programmed cell death 4 |
| 209539_at | 31.06 | 9.89 | 3.14 | + | ARHGEF6 | Rac/Cdc42 guanine nucleotide exchange factor |
| | | | | | | (GEF) 6 |
| 212593_s_at | 39.35 | 12.82 | 3.07 | + | PDCD4 | programmed cell death 4 |
| 204947_at | 50.65 | 16.65 | 3.04 | − | E2F1 | E2F transcription factor 1 |
| 201111_at | 18.77 | 6.18 | 3.04 | − | CSE1L | CSE1 chromosome segregation 1-like |
| 201636_at | 6.94 | 2.34 | 2.97 | − | FXR1 | fragile X mental retardation, autosomal homolog 1 |
| 204933_s_at | 133.57 | 45.18 | 2.96 | + | TNFRSF11B | tumor necrosis factor receptor superfamily, |
| | | | | | | member 11b |
| 220048_at | 3.61 | 1.28 | 2.82 | − | EDAR | ectodysplasin A receptor |
| 210766_s_at | 12.50 | 4.54 | 2.75 | − | CSE1L | CSE1 chromosome segregation 1-like (yeast) |
| 221567_at | 18.12 | 6.81 | 2.66 | − | NOL3 | nucleolar protein 3 (apoptosis repressor with |
| | | | | | | CARD domain) |
| 213829_x_at | 6.73 | 2.54 | 2.65 | − | TNFRSF6B | tumor necrosis factor receptor superfamily, |
| | | | | | | member 6b, decoy |
| 201112_s_at | 7.18 | 2.79 | 2.57 | − | CSE1L | CSE1 chromosome segregation 1-like |
| 212353_at | 27.06 | 10.77 | 2.51 | − | SULF1 | sulfatase 1 |
| 208822_s_at | 4.48 | 1.81 | 2.47 | − | DAP3 | death associated protein 3 |
| 209831_x_at | 6.29 | 2.59 | 2.43 | + | DNASE2 | deoxyribonuclease II, lysosomal |
| 203187_at | 7.63 | 3.21 | 2.37 | + | DOCK1 | dedicator of cytokinesis 1 |
| 209462_at | 87.55 | 36.92 | 2.37 | − | APLP1 | amyloid beta (A4) precursor-like protein 1 |
| 210164_at | 54.43 | 23.24 | 2.34 | + | GZMB | granzyme B |
| 203005_at | 4.52 | 1.98 | 2.29 | − | LTBR | lymphotoxin beta receptor |
| 209239_at | 8.01 | 3.57 | 2.24 | + | NFKB1 | nuclear factor of kappa light polypeptide gene |
| | | | | | | enhancer in B-cells 1 (p105) |
| 202535_at | 14.80 | 6.72 | 2.20 | − | FADD | Fas (TNFRSF6)-associated via death domain |
| 209803_s_at | 48.69 | 22.44 | 2.17 | − | PHLDA2 | pleckstrin homology-like domain, family A, |
| | | | | | | member 2 |
| 204513_s_at | 9.17 | 4.29 | 2.14 | + | ELMO1 | engulfment and cell motility 1 (ced-12 homolog, |
| | | | | | | C. elegans) |
| 210538_s_at | 26.69 | 12.54 | 2.13 | + | BIRC3 | baculoviral IAP repeat-containing 3 |
| 217840_at | 3.44 | 1.62 | 2.12 | − | DDX41 | DEAD (Asp-Glu-Ala-Asp) box polypeptide 41 |
| 208402_at | 34.33 | 16.37 | 2.10 | + | IL17 | interleukin 17 (cytotoxic T-lymphocyte- |
| | | | | | | associated serine esterase 8) |
| 214992_s_at | 7.20 | 3.46 | 2.08 | + | DNASE2 | deoxyribonuclease II, lysosomal |
| 209201_x_at | 28.29 | 13.71 | 2.06 | + | CXCR4 | chemokine (C—X—C motif) receptor 4 |
| 2028_s_at | 2.14 | 1.06 | 2.01 | − | E2F1 | E2F transcription factor 1 |
| 201588_at | 1.13 | 0.56 | 2.01 | − | TXNL1 | thioredoxin-like 1 |
| 203836_s_at | 6.48 | 3.29 | 1.97 | + | MAP3K5 | mitogen-activated protein kinase kinase kinase 5 |
| 215719_x_at | 20.18 | 10.30 | 1.96 | + | FAS | Fas (TNF receptor superfamily, member 6) |

Regulation of cell cycle

| | | | | | | |
|---|---|---|---|---|---|---|
| 204817_at | 33.18 | 8.90 | 3.73 | − | ESPL1 | extra spindle poles like 1 |
| 38158_at | 22.48 | 6.60 | 3.41 | − | ESPL1 | extra spindle poles like 1 |
| 214710_s_at | 22.24 | 7.19 | 3.10 | − | CCNB1 | cyclin B1 |
| 201076_at | 7.52 | 2.43 | 3.09 | + | NHP2L1 | NHP2 non-histone chromosome protein 2-like 1 |
| 212426_s_at | 7.86 | 2.55 | 3.08 | − | YWHAQ | tyrosine 3-monooxygenase/tryptophan 5- |
| | | | | | | monooxygenase activation protein |
| 204009_s_at | 7.79 | 2.53 | 3.08 | − | KRAS | v-Ki-ras2 Kirsten rat sarcoma viral oncogene |
| | | | | | | homolog |
| 204947_at | 46.18 | 15.18 | 3.04 | − | E2F1 | E2F transcription factor 1 |
| 201947_s_at | 7.00 | 2.30 | 3.04 | − | CCT2 | chaperonin containing TCP1, subunit 2 (beta) |
| 201601_x_at | 24.46 | 8.16 | 3.00 | + | IFITM1 | interferon induced transmembrane protein 1 (9- |
| | | | | | | 27) |
| 204822_at | 42.21 | 14.49 | 2.91 | − | TTK | TTK protein kinase |
| 204015_s_at | 71.73 | 24.75 | 2.90 | + | DUSP4 | dual specificity phosphatase 4 |
| 220407_s_at | 17.06 | 6.36 | 2.68 | + | TGFB2 | transforming growth factor, beta 2 |
| 209096_at | 7.11 | 2.77 | 2.57 | − | UBE2V2 | ubiquitin-conjugating enzyme E2 variant 2 |
| 204826_at | 10.95 | 4.33 | 2.53 | − | CCNF | cyclin F |
| 212022_s_at | 35.48 | 14.44 | 2.46 | − | MKI67 | antigen identified by monoclonal antibody Ki-67 |
| 202647_s_at | 8.26 | 3.41 | 2.42 | − | NRAS | neuroblastoma RAS viral (v-ras) oncogene |
| | | | | | | homolog |
| 206404_at | 26.09 | 10.98 | 2.38 | + | FGF9 | fibroblast growth factor 9 (glia-activating factor) |
| 202705_at | 25.47 | 10.74 | 2.37 | − | CCNB2 | cyclin B2 |
| 202870_s_at | 25.76 | 11.32 | 2.28 | − | CDC20 | CDC20 cell division cycle 20 homolog (S. cerevisiae) |
| 205842_s_at | 11.21 | 4.96 | 2.26 | + | JAK2 | Janus kinase 2 (a protein tyrosine kinase) |
| 214022_s_at | 13.99 | 6.25 | 2.24 | + | IFITM1 | interferon induced transmembrane protein 1 (9- |
| | | | | | | 27) |
| 211251_x_at | 6.21 | 2.96 | 2.10 | + | NFYC | nuclear transcription factor Y, gamma |
| 204014_at | 48.13 | 23.03 | 2.09 | + | DUSP4 | dual specificity phosphatase 4 |
| 212781_at | 3.04 | 1.50 | 2.02 | − | RBBP6 | retinoblastoma binding protein 6 |
| 2028_s_at | 1.95 | 0.97 | 2.01 | − | E2F1 | E2F transcription factor 1 |

Protein amino acid phosphorylation

| | | | | | | |
|---|---|---|---|---|---|---|
| 208079_s_at | 120.73 | 28.59 | 4.22 | − | STK6 | serine/threonine kinase 6 |
| 204092_s_at | 62.39 | 17.05 | 3.66 | − | STK6 | serine/threonine kinase 6 |
| 204641_at | 143.19 | 40.31 | 3.55 | − | NEK2 | NIMA (never in mitosis gene a)-related kinase 2 |
| 210754_s_at | 22.18 | 6.89 | 3.22 | + | LYN | v-yes-1 Yamaguchi sarcoma viral related |
| | | | | | | oncogene homolog |
| 218909_at | 6.75 | 2.10 | 3.21 | − | RPS6KC1 | ribosomal protein S6 kinase, 52 kDa, |
| | | | | | | polypeptide 1 |
| 202543_s_at | 21.69 | 6.87 | 3.16 | − | GMFB | glia maturation factor, beta |
| 204825_at | 43.55 | 13.94 | 3.12 | − | MELK | maternal embryonic leucine zipper kinase |
| 203213_at | 52.80 | 17.25 | 3.06 | − | CDC2 | Cell division cycle 2, G1 to S and G2 to M |
| 204822_at | 63.55 | 21.81 | 2.91 | − | TTK | TTK protein kinase |
| 204171_at | 23.52 | 8.48 | 2.77 | − | RPS6KB1 | ribosomal protein S6 kinase, 70 kDa, |
| | | | | | | polypeptide 1 |
| 218764_at | 12.75 | 4.71 | 2.71 | + | PRKCH | protein kinase C, eta |
| 216598_s_at | 118.88 | 46.84 | 2.54 | + | CCL2 | chemokine (C—C motif) ligand 2 |
| 203755_at | 19.43 | 7.95 | 2.44 | − | BUB1B | BUB1 budding uninhibited by benzimidazoles 1 |
| | | | | | | homolog beta (yeast) |
| 208944_at | 24.04 | 9.85 | 2.44 | + | TGFBR2 | transforming growth factor, beta receptor II |
| | | | | | | (70/80 kDa) |
| 220038_at | 46.82 | 19.30 | 2.43 | + | SGK3 | serum/glucocorticoid regulated kinase family, |
| | | | | | | member 3 |
| 209642_at | 33.53 | 13.87 | 2.42 | − | BUB1 | BUB1 budding uninhibited by benzimidazoles 1 |
| | | | | | | homolog (yeast) |
| 207957_s_at | 73.49 | 30.64 | 2.40 | + | ATP6AP1 | ATPase, H+ transporting, lysosomal accessory |
| | | | | | | protein 1 |
| 208018_s_at | 11.78 | 5.00 | 2.36 | + | HCK | hemopoietic cell kinase |
| 212486_s_at | 30.72 | 13.32 | 2.31 | + | FYN | FYN oncogene related to SRC, FGR, YES |
| 216033_s_at | 44.93 | 19.72 | 2.28 | + | FYN | FYN oncogene related to SRC, FGR, YES |
| 205842_s_at | 16.88 | 7.47 | 2.26 | + | JAK2 | Janus kinase 2 (a protein tyrosine kinase) |
| 219813_at | 16.04 | 7.16 | 2.24 | + | LATS1 | LATS, large tumor suppressor, homolog 1 |
| | | | | | | (Drosophila) |
| 220987_s_at | 4.46 | 2.03 | 2.19 | − | NUAK2 | NUAK family, SNF1-like kinase, 2 |
| 212530_at | 3.13 | 1.44 | 2.17 | − | NEK7 | NIMA (never in mitosis gene a)-related kinase 7 |
| 209282_at | 8.49 | 4.15 | 2.04 | + | PRKD2 | protein kinase D2 |
| 202200_s_at | 3.80 | 1.88 | 2.02 | − | SRPK1 | SFRS protein kinase 1 |
| 203836_s_at | 8.90 | 4.51 | 1.97 | + | MAP3K5 | mitogen-activated protein kinase kinase kinase 5 |

Cytokinesis

| | | | | | | |
|---|---|---|---|---|---|---|
| 204817_at | 17.44 | 4.68 | 3.73 | − | ESPL1 | extra spindle poles like 1 |
| 204641_at | 49.99 | 14.07 | 3.55 | − | NEK2 | NIMA (never in mitosis gene a)-related kinase 2 |
| 38158_at | 11.82 | 3.47 | 3.41 | − | ESPL1 | extra spindle poles like 1 |
| 218009_s_at | 18.49 | 5.67 | 3.26 | − | PRC1 | protein regulator of cytokinesis 1 |
| 214710_s_at | 11.69 | 3.78 | 3.10 | − | CCNB1 | cyclin B1 |
| 203213_at | 18.43 | 6.02 | 3.06 | − | CDC2 | Cell division cycle 2, G1 to S and G2 to M |
| 205046_at | 43.34 | 16.80 | 2.58 | − | CENPE | centromere protein E, 312 kDa |
| 204826_at | 5.76 | 2.27 | 2.53 | − | CCNF | cyclin F |
| 201589_at | 3.22 | 1.32 | 2.44 | − | SMC1L1 | SMC1 structural maintenance of chromosomes |
| | | | | | | 1-like 1 |
| 200815_s_at | 2.27 | 0.94 | 2.41 | − | PAFAH1B1 | platelet-activating factor acetylhydrolase, |
| | | | | | | isoform lb, alpha subunit 45 kDa |
| 202705_at | 13.39 | 5.64 | 2.37 | − | CCNB2 | cyclin B2 |
| 200726_at | 1.62 | 0.70 | 2.32 | − | PPP1CC | protein phosphatase 1, catalytic subunit, |
| | | | | | | gamma isoform |
| 202870_s_at | 13.54 | 5.95 | 2.28 | − | CDC20 | CDC20 cell division cycle 20 homolog (S. cerevisiae) |
| 201897_s_at | 3.37 | 1.58 | 2.14 | − | CKS1B | CDC28 protein kinase regulatory subunit 1B |
| 204170_s_at | 8.07 | 3.89 | 2.07 | − | CKS2 | CDC28 protein kinase regulatory subunit 2 |
| 213743_at | 1.39 | 0.70 | 1.99 | − | CCNT2 | cyclin T2 |

Cell motility

| | | | | | | |
|---|---|---|---|---|---|---|
| 207165_at | 35.78 | 9.04 | 3.96 | − | HMMR | hyaluronan-mediated motility receptor |
| | | | | | | (RHAMM) |
| 206983_at | 32.30 | 9.85 | 3.28 | + | CCR6 | chemokine (C—C motif) receptor 6 |
| 211719_x_at | 5.66 | 1.97 | 2.87 | − | FN1 | fibronectin 1 |
| 211577_s_at | 18.73 | 7.25 | 2.58 | + | IGF1 | insulin-like growth factor 1 |
| 210495_x_at | 3.69 | 1.49 | 2.47 | − | FN1 | fibronectin 1 |
| 208991_at | 5.91 | 2.43 | 2.43 | + | STAT3 | signal transducer and activator of transcription 3 |
| 200815_s_at | 3.18 | 1.32 | 2.41 | − | PAFAH1B1 | platelet-activating factor acetylhydrolase, |
| | | | | | | isoform lb, alpha subunit 45 kDa |
| 200973_s_at | 10.68 | 4.50 | 2.37 | + | TSPAN3 | tetraspanin 3 |
| 216442_x_at | 3.76 | 1.65 | 2.27 | − | FN1 | fibronectin 1 |
| 209540_at | 25.74 | 11.37 | 2.26 | + | IGF1 | insulin-like growth factor 1 (somatomedin C) |
| 205842_s_at | 8.27 | 3.66 | 2.26 | + | JAK2 | Janus kinase 2 (a protein tyrosine kinase) |
| 209083_at | 19.05 | 8.86 | 2.15 | + | CORO1A | coronin, actin binding protein, 1A |
| 204513_s_at | 6.17 | 2.89 | 2.14 | + | ELMO1 | engulfment and cell motility 1 (ced-12 homolog, |
| | | | | | | C. elegans) |
| 207008_at | 32.40 | 15.61 | 2.08 | + | IL8RB | interleukin 8 receptor, beta |
| 208992_s_at | 13.84 | 6.76 | 2.05 | + | STAT3 | signal transducer and activator of transcription 3 |
| 213101_s_at | 2.59 | 1.28 | 2.03 | − | ACTR3 | ARP3 actin-related protein 3 homolog (yeast) |
| 208679_s_at | 3.77 | 1.93 | 1.96 | + | ARPC2 | actin related protein 2/3 complex, subunit 2, |
| | | | | | | 34 kDa |

Cell cycle

| | | | | | | |
|---|---|---|---|---|---|---|
| 201664_at | 18.20 | 4.00 | 4.55 | − | SMC4L1 | SMC4 structural maintenance of chromosomes |
| | | | | | | 4-like 1 |
| 208079_s_at | 84.89 | 20.10 | 4.22 | − | STK6 | serine/threonine kinase 6 |
| 204092_s_at | 43.87 | 11.99 | 3.66 | − | STK6 | serine/threonine kinase 6 |
| 215623_x_at | 16.82 | 5.18 | 3.25 | − | SMC4L1 | SMC4 structural maintenance of chromosomes |
| | | | | | | 4-like 1 |
| 218663_at | 28.34 | 9.46 | 2.99 | − | HCAP-G | chromosome condensation protein G |
| 203362_s_at | 35.05 | 12.46 | 2.81 | − | MAD2L1 | MAD2 mitotic arrest deficient-like 1 |
| 32137_at | 4.45 | 1.67 | 2.67 | − | JAG2 | jagged 2 |
| 203755_at | 13.66 | 5.59 | 2.44 | − | BUB1B | BUB1 budding uninhibited by benzimidazoles 1 |
| | | | | | | homolog beta |
| 201589_at | 6.49 | 2.66 | 2.44 | − | SMC1L1 | SMC1 structural maintenance of chromosomes |
| | | | | | | 1-like 1 |
| 209642_at | 23.58 | 9.75 | 2.42 | − | BUB1 | BUB1 budding uninhibited by benzimidazoles 1 |
| | | | | | | homolog |
| 204496_at | 11.23 | 4.77 | 2.35 | − | STRN3 | striatin, calmodulin binding protein 3 |
| 218662_s_at | 10.87 | 4.96 | 2.19 | − | HCAP-G | chromosome condensation protein G |
| 201663_s_at | 8.91 | 4.21 | 2.12 | − | SMC4L1 | SMC4 structural maintenance of chromosomes |
| | | | | | | 4-like 1 |
| 204170_s_at | 16.25 | 7.83 | 2.07 | − | CKS2 | CDC28 protein kinase regulatory subunit 2 |
| 206499_s_at | 3.35 | 1.62 | 2.07 | + | RCC1 | regulator of chromosome condensation 1 |
| 202214_s_at | 2.35 | 1.16 | 2.03 | + | CUL4B | cullin 4B |
| 213743_at | 2.80 | 1.41 | 1.99 | − | CCNT2 | cyclin T2 |

Cell surface receptor linked signal transduction

| | | | | | | |
|---|---|---|---|---|---|---|
| 206150_at | 36.90 | 10.33 | 3.57 | + | TNFRSF7 | tumor necrosis factor receptor superfamily, |
| | | | | | | member 7 |
| 205926_at | 9.28 | 2.66 | 3.49 | + | IL27RA | interleukin 27 receptor, alpha |
| 212587_s_at | 23.07 | 6.96 | 3.32 | + | PTPRC | protein tyrosine phosphatase, receptor type, C |
| 201601_x_at | 14.65 | 4.89 | 3.00 | + | IFITM1 | interferon induced transmembrane protein 1 (9- |
| | | | | | | 27) |
| 211000_s_at | 12.04 | 4.40 | 2.73 | + | IL6ST | interleukin 6 signal transducer (gp130, |
| | | | | | | oncostatin M receptor) |
| 214470_at | 33.53 | 13.03 | 2.57 | + | KLRB1 | killer cell lectin-like receptor subfamily B, |
| | | | | | | member 1 |
| 222062_at | 29.79 | 12.76 | 2.33 | + | IL27RA | interleukin 27 receptor, alpha |
| 214022_s_at | 8.38 | 3.74 | 2.24 | + | IFITM1 | interferon induced transmembrane protein 1 (9- |
| | | | | | | 27) |
| 202535_at | 8.08 | 3.67 | 2.20 | − | FADD | Fas (TNFRSF6)-associated via death domain |
| 210538_s_at | 14.57 | 6.84 | 2.13 | + | BIRC3 | baculoviral IAP repeat-containing 3 |

Mitosis

| | | | | | | |
|---|---|---|---|---|---|---|
| 201664_at | 8.10 | 1.78 | 4.55 | − | SMC4L1 | SMC4 structural maintenance of chromosomes |
| | | | | | | 4-like 1 |
| 208079_s_at | 37.77 | 8.94 | 4.22 | − | STK6 | serine/threonine kinase 6 |
| 204092_s_at | 19.52 | 5.33 | 3.66 | − | STK6 | serine/threonine kinase 6 |
| 215623_x_at | 7.48 | 2.31 | 3.25 | − | SMC4L1 | SMC4 structural maintenance of chromosomes |
| | | | | | | 4-like 1 |
| 209172_s_at | 9.26 | 2.86 | 3.24 | − | CENPF | centromere protein F, 350/400ka (mitosin) |
| 214710_s_at | 10.47 | 3.38 | 3.10 | − | CCNB1 | cyclin B1 |
| 203213_at | 16.52 | 5.40 | 3.06 | − | CDC2 | Cell division cycle 2, G1 to S and G2 to M |
| 218663_at | 12.61 | 4.21 | 2.99 | − | HCAP-G | chromosome condensation protein G |
| 203362_s_at | 15.59 | 5.55 | 2.81 | − | MAD2L1 | MAD2 mitotic arrest deficient-like 1 |
| 204826_at | 5.16 | 2.04 | 2.53 | − | CCNF | cyclin F |
| 203755_at | 6.08 | 2.49 | 2.44 | − | BUB1B | BUB1 budding uninhibited by benzimidazoles 1 |
| | | | | | | homolog beta |
| 209642_at | 10.49 | 4.34 | 2.42 | − | BUB1 | BUB1 budding uninhibited by benzimidazoles 1 |
| | | | | | | homolog |
| 200815_s_at | 2.03 | 0.84 | 2.41 | − | PAFAH1B1 | platelet-activating factor acetylhydrolase, |
| | | | | | | isoform lb, alpha subunit 45 kDa |
| 202705_at | 12.00 | 5.06 | 2.37 | − | CCNB2 | cyclin B2 |
| 209408_at | 6.66 | 2.87 | 2.32 | − | KIF2C | kinesin family member 2C |
| 202870_s_at | 12.13 | 5.33 | 2.28 | − | CDC20 | CDC20 cell division cycle 20 homolog (S. cerevisiae) |
| 218662_s_at | 4.83 | 2.21 | 2.19 | − | HCAP-G | chromosome condensation protein G |
| 209083_at | 12.16 | 5.65 | 2.15 | + | CORO1A | coronin, actin binding protein, 1A |
| 201663_s_at | 3.97 | 1.87 | 2.12 | − | SMC4L1 | SMC4 structural maintenance of chromosomes |
| | | | | | | 4-like 1 |
| 206499_s_at | 1.49 | 0.72 | 2.07 | + | RCC1 | regulator of chromosome condensation 1 |

Intracellular protein transport

| | | | | | | |
|---|---|---|---|---|---|---|
| 201216_at | 22.62 | 4.46 | 5.07 | + | ERP29 | endoplasmic reticulum protein 29 |
| 211779_x_at | 10.48 | 3.08 | 3.40 | + | AP2A2 | adaptor-related protein complex 2, alpha 2 |
| | | | | | | subunit |
| 212159_x_at | 11.53 | 3.60 | 3.21 | + | AP2A2 | adaptor-related protein complex 2, alpha 2 |
| | | | | | | subunit |
| 201088_at | 51.35 | 16.82 | 3.05 | − | KPNA2 | karyopherin alpha 2 |
| 201111_at | 32.61 | 10.74 | 3.04 | − | CSE1L | CSE1 chromosome segregation 1-like |
| 204478_s_at | 9.39 | 3.13 | 3.00 | − | RABIF | RAB interacting factor |
| 203311_s_at | 15.15 | 5.20 | 2.91 | + | ARF6 | ADP-ribosylation factor 6 |
| 214337_at | 105.30 | 36.24 | 2.91 | − | COPA | coatomer protein complex, subunit alpha |
| 204974_at | 52.86 | 18.62 | 2.84 | − | RAB3A | RAB3A, member RAS oncogene family |
| 202630_at | 22.63 | 8.05 | 2.81 | − | APPBP2 | amyloid beta precursor protein (cytoplasmic tail) |
| | | | | | | binding protein 2 |
| 208819_at | 4.68 | 1.68 | 2.78 | + | RAB8A | RAB8A, member RAS oncogene family |
| 210766_s_at | 21.71 | 7.89 | 2.75 | − | CSE1L | CSE1 chromosome segregation 1-like |
| 209268_at | 9.70 | 3.53 | 2.74 | − | VPS45A | vacuolar protein sorting 45A |
| 201831_s_at | 9.56 | 3.50 | 2.73 | + | VDP | vesicle docking protein p115 |
| 218360_at | 16.60 | 6.43 | 2.58 | − | RAB22A | RAB22A, member RAS oncogene family |
| 201112_s_at | 12.48 | 4.85 | 2.57 | − | CSE1L | CSE1 chromosome segregation 1-like |
| 203679_at | 11.96 | 4.69 | 2.55 | + | TMED1 | transmembrane emp24 protein transport |
| | | | | | | domain containing 1 |
| 218755_at | 32.63 | 12.95 | 2.52 | − | KIF20A | kinesin family member 20A |
| 209238_at | 12.00 | 4.78 | 2.51 | − | STX3A | syntaxin 3A |
| 204017_at | 24.75 | 10.31 | 2.40 | − | KDELR3 | KDEL (Lys-Asp-Glu-Leu) endoplasmic reticulum |
| | | | | | | protein retention receptor 3 |
| 202395_at | 16.99 | 7.11 | 2.39 | − | NSF | N-ethylmaleimide-sensitive factor |
| 221014_s_at | 7.83 | 3.53 | 2.22 | − | RAB33B | RAB33B, member RAS oncogene family |
| 212652_s_at | 3.70 | 1.73 | 2.14 | − | SNX4 | sorting nexin 4 |
| 212103_at | 4.16 | 1.95 | 2.13 | + | KPNA6 | Karyopherin alpha 6 (importin alpha 7) |
| 204477_at | 9.92 | 4.67 | 2.13 | − | RABIF | RAB interacting factor |
| 201097_s_at | 2.72 | 1.28 | 2.12 | − | ARF4 | ADP-ribosylation factor 4 |
| 212635_at | 6.06 | 2.88 | 2.10 | − | TNPO1 | Transportin 1 |
| 203544_s_at | 8.14 | 3.93 | 2.07 | − | STAM | signal transducing adaptor molecule (SH3 |
| | | | | | | domain and ITAM motif) 1 |
| 211762_s_at | 19.76 | 9.65 | 2.05 | − | KPNA2 | karyopherin alpha 2 (RAG cohort 1, importin |
| | | | | | | alpha 1) |
| 200614_at | 11.87 | 5.87 | 2.02 | − | CLTC | clathrin, heavy polypeptide (Hc) |
| 208732_at | 8.12 | 4.07 | 2.00 | − | RAB2 | RAB2, member RAS oncogene family |
| 200699_at | 8.38 | 4.29 | 1.95 | − | KDELR2 | KDEL (Lys-Asp-Glu-Leu) endoplasmic reticulum |
| | | | | | | protein retention receptor 2 |

Mitotic chromosome segregation

| | | | | | | |
|---|---|---|---|---|---|---|
| 201664_at | 6.77 | 1.49 | 4.55 | − | SMC4L1 | SMC4 structural maintenance of chromosomes |
| | | | | | | 4-like 1 |
| 204817_at | 13.07 | 3.51 | 3.73 | − | ESPL1 | extra spindle poles like 1 |
| 38158_at | 8.85 | 2.60 | 3.41 | − | ESPL1 | extra spindle poles like 1 |
| 215623_x_at | 6.26 | 1.93 | 3.25 | − | SMC4L1 | SMC4 structural maintenance of chromosomes |
| | | | | | | 4-like 1 |
| 201589_at | 2.41 | 0.99 | 2.44 | − | SMC1L1 | SMC1 structural maintenance of chromosomes |
| | | | | | | 1-like 1 |
| 201663_s_at | 3.32 | 1.57 | 2.12 | − | SMC4L1 | SMC4 structural maintenance of chromosomes |
| | | | | | | 4-like 1 |

Ubiquitin-dependent protein catabolism

| | | | | | | |
|---|---|---|---|---|---|---|
| 201178_at | 10.32 | 2.73 | 3.79 | + | FBXO7 | F-box protein 7 |
| 202244_at | 9.40 | 2.71 | 3.48 | − | PSMB4 | proteasome (prosome, macropain) subunit, beta |
| | | | | | | type, 4 |
| 211702_s_at | 20.08 | 7.60 | 2.64 | − | USP32 | ubiquitin specific peptidase 32 |
| 221519_at | 5.75 | 2.22 | 2.58 | + | FBXW4 | F-box and WD-40 domain protein 4 |
| 202981_x_at | 9.35 | 3.90 | 2.40 | − | SIAH1 | seven in absentia homolog 1 (Drosophila) |
| 209040_s_at | 46.23 | 19.42 | 2.38 | + | PSMB8 | proteasome (prosome, macropain) subunit, beta |
| | | | | | | type, 8 |
| 208805_at | 11.48 | 4.83 | 2.38 | − | PSMA6 | proteasome (prosome, macropain) subunit, |
| | | | | | | alpha type, 6 |
| 202243_s_at | 6.60 | 2.87 | 2.30 | − | PSMB4 | proteasome (prosome, macropain) subunit, beta |
| | | | | | | type, 4 |
| 202870_s_at | 46.10 | 20.26 | 2.28 | − | CDC20 | CDC20 cell division cycle 20 homolog (S. cerevisiae) |
| 208760_at | 10.11 | 4.70 | 2.15 | − | UBE2I | Ubiquitin-conjugating enzyme E2I |
| 201317_s_at | 5.90 | 2.77 | 2.13 | − | PSMA2 | proteasome (prosome, macropain) subunit, |
| | | | | | | alpha type, 2 |

DNA repair

| | | | | | | |
|---|---|---|---|---|---|---|
| 219510_at | 16.77 | 4.57 | 3.67 | − | POLQ | polymerase (DNA directed), theta |
| 213520_at | 157.23 | 44.55 | 3.53 | − | RECQL4 | RecQ protein-like 4 |
| 219502_at | 12.24 | 4.08 | 3.00 | − | NEIL3 | nei endonuclease VIII-like 3 |
| 204146_at | 29.05 | 10.24 | 2.84 | − | RAD51AP1 | RAD51 associated protein 1 |
| 204558_at | 53.36 | 20.63 | 2.59 | − | RAD54L | RAD54-like |
| 204531_s_at | 11.12 | 4.52 | 2.46 | − | BRCA1 | breast cancer 1, early onset |
| 201589_at | 5.45 | 2.23 | 2.44 | − | SMC1L1 | SMC1 structural maintenance of chromosomes |
| | | | | | | 1-like 1 |
| 218397_at | 5.64 | 2.56 | 2.21 | − | FANCL | Fanconi anemia, complementation group L |
| 213734_at | 6.10 | 2.79 | 2.18 | − | WSB2 | WD repeat and SOCS box-containing 2 |

Induction of apoptosis

| | | | | | | |
|---|---|---|---|---|---|---|
| 208905_at | 14.07 | 3.28 | 4.29 | − | CYCS | cytochrome c, somatic |
| 206150_at | 72.98 | 20.43 | 3.57 | + | TNFRSF7 | tumor necrosis factor receptor superfamily, |
| | | | | | | member 7 |
| 209448_at | 24.65 | 11.28 | 2.19 | − | HTATIP2 | HIV-1 Tat interactive protein 2, 30 kDa |
| 209929_s_at | 4.91 | 2.49 | 1.97 | − | IKBKG | inhibitor of kappa light polypeptide gene |
| | | | | | | enhancer in B-cells, kinase gamma |
| 215719_x_at | 21.79 | 11.12 | 1.96 | + | FAS | Fas (TNF receptor superfamily, member 6) |

Immune response

| | | | | | | |
|---|---|---|---|---|---|---|
| 206150_at | 22.64 | 6.34 | 3.57 | + | TNFRSF7 | tumor necrosis factor receptor superfamily, |
| | | | | | | member 7 |
| 215633_x_at | 17.75 | 5.04 | 3.52 | + | LST1 | leukocyte specific transcript 1 |
| 205926_at | 5.69 | 1.63 | 3.49 | + | IL27RA | interleukin 27 receptor, alpha |
| 210629_x_at | 7.36 | 2.12 | 3.47 | + | LST1 | leukocyte specific transcript 1 |
| 204670_x_at | 13.15 | 3.95 | 3.33 | + | HLA-DRB1 | major histocompatibility complex, class II, DR |
| | | | | | | beta 1 |
| 211582_x_at | 17.49 | 5.72 | 3.06 | + | LST1 | leukocyte specific transcript 1 |
| 210982_s_at | 31.37 | 10.27 | 3.05 | + | HLA-DRA | major histocompatibility complex, class II, DR |
| | | | | | | alpha |
| 209312_x_at | 13.65 | 4.51 | 3.02 | + | HLA-DRB1 | major histocompatibility complex, class II, DR |
| | | | | | | beta 1 |
| 213226_at | 10.10 | 3.37 | 3.00 | − | CCNA2 | Cyclin A2 |
| 201601_x_at | 8.98 | 3.00 | 3.00 | + | IFITM1 | interferon induced transmembrane protein 1 (9-27) |
| 208894_at | 24.35 | 8.56 | 2.84 | + | HLA-DRA | major histocompatibility complex, class II, DR |
| | | | | | | alpha |
| 211991_s_at | 17.17 | 6.07 | 2.83 | + | HLA-DPA1 | major histocompatibility complex, class II, DP |
| | | | | | | alpha 1 |
| 215193_x_at | 17.46 | 6.18 | 2.82 | + | HLA-DRB1 | major histocompatibility complex, class II, DR |
| | | | | | | beta 1 |
| 217478_s_at | 9.71 | 3.45 | 2.82 | + | HLA-DMA | major histocompatibility complex, class II, DM |
| | | | | | | alpha |
| 210072_at | 31.12 | 11.12 | 2.80 | + | CCL19 | chemokine (C—C motif) ligand 19 |
| 200904_at | 8.21 | 2.98 | 2.76 | + | HLA-E | major histocompatibility complex, class I, E |
| 211000_s_at | 7.38 | 2.70 | 2.73 | + | IL6ST | interleukin 6 signal transducer (gp130, |
| | | | | | | oncostatin M receptor) |
| 211581_x_at | 12.05 | 4.50 | 2.68 | + | LST1 | leukocyte specific transcript 1 |
| 209823_x_at | 21.88 | 8.17 | 2.68 | + | HLA-DQB1 | major histocompatibility complex, class II, DQ |
| | | | | | | beta 1 |
| 207850_at | 17.82 | 6.79 | 2.63 | + | CXCL3 | chemokine (C—X—C motif) ligand 3 |
| 208306_x_at | 8.90 | 3.40 | 2.62 | + | HLA-DRB1 | Major histocompatibility complex, class II, DR |
| | | | | | | beta 3 |
| 203010_at | 3.23 | 1.27 | 2.54 | + | STAT5A | signal transducer and activator of transcription |
| | | | | | | 5A |
| 200905_x_at | 3.98 | 1.58 | 2.52 | + | HLA-E | major histocompatibility complex, class I, E |
| 201288_at | 6.88 | 2.73 | 2.52 | + | ARHGDIB | Rho GDP dissociation inhibitor (GDI) beta |
| 215784_at | 30.48 | 12.17 | 2.50 | + | CD1E | CD1E antigen, e polypeptide |
| 205544_s_at | 26.20 | 10.46 | 2.50 | + | CR2 | complement component (3d/Epstein Barr virus) |
| | | | | | | receptor 2 |
| 211430_s_at | 23.54 | 9.63 | 2.44 | + | IGH | immunoglobulin heavy constant gamma 1 (G1m |
| | | | | | | marker) |
| 217456_x_at | 2.67 | 1.09 | 2.44 | + | HLA-E | major histocompatibility complex, class I, E |
| 201137_s_at | 8.17 | 3.36 | 2.43 | + | HLA-DPB1 | major histocompatibility complex, class II, DP |
| | | | | | | beta 1 |
| 211529_x_at | 7.99 | 3.32 | 2.41 | + | HLA-G | HLA-G histocompatibility antigen, class I, G |
| 212592_at | 42.76 | 17.85 | 2.40 | + | IGJ | Immunoglobulin J polypeptide |
| 204470_at | 7.85 | 3.30 | 2.38 | + | CXCL1 | chemokine (C—X—C motif) ligand 1 |
| 209040_s_at | 9.49 | 3.99 | 2.38 | + | PSMB8 | proteasome (prosome, macropain) subunit, beta |
| | | | | | | type, 8 |
| 209687_at | 14.05 | 5.97 | 2.35 | + | CXCL12 | chemokine (C—X—C motif) ligand 12 |
| 222062_at | 18.27 | 7.83 | 2.33 | + | IL27RA | interleukin 27 receptor, alpha |
| 205671_s_at | 14.74 | 6.33 | 2.33 | + | HLA-DOB | major histocompatibility complex, class II, DO |
| | | | | | | beta |
| 202748_at | 4.75 | 2.04 | 2.33 | + | GBP2 | guanylate binding protein 2, interferon-inducible |
| 217767_at | 12.27 | 5.31 | 2.31 | + | C3 | complement component 3 |
| 211799_x_at | 9.65 | 4.19 | 2.30 | + | HLA-C | major histocompatibility complex, class I, C |
| 203005_at | 1.51 | 0.66 | 2.29 | − | LTBR | lymphotoxin beta receptor (TNFR superfamily, |
| | | | | | | member 3) |
| 212203_x_at | 2.79 | 1.22 | 2.28 | + | IFITM3 | interferon induced transmembrane protein 3 (1-8 U) |
| 203666_at | 5.48 | 2.43 | 2.26 | + | CXCL12 | chemokine (C—X—C motif) ligand 12 |
| 214022_s_at | 5.14 | 2.30 | 2.24 | + | IFITM1 | interferon induced transmembrane protein 1 (9-27) |
| 217014_s_at | 15.72 | 7.03 | 2.24 | + | AZGP1 | alpha-2-glycoprotein 1, zinc |
| 211911_x_at | 8.34 | 3.73 | 2.23 | + | HLA-B | major histocompatibility complex, class I, B |
| 210514_x_at | 11.98 | 5.36 | 2.23 | + | HLA-G | HLA-G histocompatibility antigen, class I, G |
| 204116_at | 6.74 | 3.09 | 2.18 | + | IL2RG | interleukin 2 receptor, gamma |
| 209619_at | 8.17 | 3.75 | 2.18 | + | CD74 | CD74 antigen |
| 208729_x_at | 7.58 | 3.54 | 2.14 | + | HLA-B | major histocompatibility complex, class I, B |
| 207323_s_at | 2.28 | 1.08 | 2.12 | + | MBP | myelin basic protein |
| 212671_s_at | 15.09 | 7.13 | 2.12 | + | HLA-DQA1 | major histocompatibility complex, class II, DQ |
| | | | | | /// HLA- | alpha 1 |
| | | | | | DQA2 | |
| 211528_x_at | 6.34 | 3.00 | 2.11 | + | HLA-G | HLA-G histocompatibility antigen, class I, G |
| 208402_at | 11.50 | 5.48 | 2.10 | + | IL17 | interleukin 17 |
| 209666_s_at | 2.11 | 1.01 | 2.08 | − | CHUK | conserved helix-loop-helix ubiquitous kinase |
| 209201_x_at | 9.47 | 4.59 | 2.06 | + | CXCR4 | chemokine (C—X—C motif) receptor 4 |
| 206641_at | 23.27 | 11.37 | 2.05 | + | TNFRSF17 | tumor necrosis factor receptor superfamily, |
| | | | | | | member 17 |
| 211734_s_at | 12.74 | 6.25 | 2.04 | + | FCER1A | Fc fragment of IgE, high affinity I, receptor for; |
| | | | | | | alpha polypeptide |
| 204806_x_at | 4.70 | 2.33 | 2.02 | + | HLA-F | major histocompatibility complex, class I, F |
| 215669_at | 3.81 | 1.90 | 2.01 | − | HLA-DRB4 | major histocompatibility complex, class II, DR |
| | | | | | | beta 4 |
| 206086_x_at | 0.71 | 0.36 | 1.98 | − | HFE | hemochromatosis |
| 209929_s_at | 1.52 | 0.77 | 1.97 | − | IKBKG | inhibitor of kappa light polypeptide gene |
| | | | | | | enhancer in B-cells, kinase gamma |
| 202992_at | 25.86 | 13.15 | 1.97 | + | C7 | complement component 7 |
| 214974_x_at | 8.97 | 4.58 | 1.96 | + | CXCL5 | chemokine (C—X—C motif) ligand 5 |
| 215719_x_at | 6.76 | 3.45 | 1.96 | + | FAS | Fas (TNF receptor superfamily, member 6) |

Protein biosynthesis

| | | | | | | |
|---|---|---|---|---|---|---|
| 211666_x_at | 56.18 | 14.56 | 3.86 | + | RPL3 | ribosomal protein L3 |
| 217747_s_at | 21.97 | 6.01 | 3.66 | + | RPS9 | ribosomal protein S9 |
| 200937_s_at | 22.70 | 6.32 | 3.59 | + | RPL5 | ribosomal protein L5 |
| 200081_s_at | 18.99 | 5.85 | 3.25 | + | RPS6 | ribosomal protein S6 |
| 201076_at | 18.95 | 6.12 | 3.09 | + | NHP2L1 | NHP2 non-histone chromosome protein 2-like 1 |
| 211938_at | 17.38 | 5.67 | 3.07 | + | EIF4B | eukaryotic translation initiation factor 4B |
| 200024_at | 20.65 | 6.95 | 2.97 | + | RPS5 | ribosomal protein S5 |
| 208887_at | 22.22 | 7.58 | 2.93 | + | EIF3S4 | eukaryotic translation initiation factor 3, subunit |
| | | | | | | 4 delta, 44 kDa |
| 213687_s_at | 7.25 | 2.48 | 2.92 | + | RPL35A | ribosomal protein L35a |
| 200036_s_at | 13.18 | 4.52 | 2.91 | + | RPL10A | ribosomal protein L10a |
| 200823_x_at | 46.07 | 15.87 | 2.90 | + | RPL29 | ribosomal protein L29 |
| 220960_x_at | 20.05 | 7.47 | 2.68 | + | RPL22 | ribosomal protein L22 |
| 211710_x_at | 6.88 | 2.58 | 2.66 | + | RPL4 | ribosomal protein L4 |
| 202247_s_at | 16.72 | 6.28 | 2.66 | + | MTA1 | metastasis associated 1 |
| 200005_at | 8.27 | 3.11 | 2.66 | + | EIF3S7 | eukaryotic translation initiation factor 3, subunit |
| | | | | | | 7 zeta, 66/67 kDa |
| 200013_at | 4.18 | 1.59 | 2.63 | + | RPL24 | ribosomal protein L24 |
| 221726_at | 12.88 | 4.90 | 2.63 | + | RPL22 | ribosomal protein L22 |
| 201258_at | 6.53 | 2.49 | 2.62 | + | RPS16 | ribosomal protein S16 |
| 213310_at | 34.83 | 13.70 | 2.54 | − | EIF2C2 | Eukaryotic translation initiation factor 2C, 2 |
| 200074_s_at | 11.82 | 4.67 | 2.53 | + | RPL14 | ribosomal protein L14 |
| 200869_at | 29.52 | 11.75 | 2.51 | + | RPL18A | ribosomal protein L18a |
| 218270_at | 7.18 | 2.92 | 2.46 | + | MRPL24 | mitochondrial ribosomal protein L24 |
| 209609_s_at | 10.14 | 4.22 | 2.40 | − | MRPL9 | mitochondrial ribosomal protein L9 |
| 201254_x_at | 2.75 | 1.19 | 2.31 | + | RPS6 | ribosomal protein S6 |
| 201154_x_at | 5.49 | 2.40 | 2.29 | + | RPL4 | ribosomal protein L4 |
| 200010_at | 5.97 | 2.63 | 2.27 | + | RPL11 | Ribosomal protein L11 |
| 201064_s_at | 7.61 | 3.38 | 2.25 | + | PABPC4 | poly(A) binding protein, cytoplasmic 4 (inducible |
| | | | | | | form) |
| 200022_at | 8.61 | 3.89 | 2.21 | + | RPL18 | ribosomal protein L18 |
| 212450_at | 10.26 | 4.66 | 2.20 | − | KIAA0256 | KIAA0256 gene product |
| 213414_s_at | 3.95 | 1.83 | 2.16 | + | RPS19 | ribosomal protein S19 |
| 221798_x_at | 0.88 | 0.41 | 2.16 | − | RPS2 | Ribosomal protein S2 |
| 211937_at | 8.65 | 4.05 | 2.14 | + | EIF4B | eukaryotic translation initiation factor 4B |
| 208264_s_at | 8.58 | 4.08 | 2.10 | − | EIF3S1 | eukaryotic translation initiation factor 3, subunit |
| | | | | | | 1 alpha, 35 kDa |
| 200012_x_at | 8.42 | 4.04 | 2.08 | + | RPL21 | ribosomal protein L21 |
| 200858_s_at | 5.06 | 2.44 | 2.07 | + | RPS8 | ribosomal protein S8 |
| 209134_s_at | 3.91 | 1.95 | 2.01 | + | RPS6 | ribosomal protein S6 |
| 208695_s_at | 0.96 | 0.49 | 1.97 | − | RPL39 | ribosomal protein L39 |

DNA replication

| | | | | | | |
|---|---|---|---|---|---|---|
| 219105_x_at | 18.23 | 5.57 | 3.27 | − | ORC6L | origin recognition complex, subunit 6 homolog- |
| | | | | | | like |
| 201890_at | 37.16 | 11.68 | 3.18 | − | RRM2 | ribonucleotide reductase M2 polypeptide |
| 211577_s_at | 20.37 | 7.88 | 2.58 | + | IGF1 | insulin-like growth factor 1 (somatomedin C) |
| 221521_s_at | 44.39 | 17.27 | 2.57 | − | Pfs2 | DNA replication complex GINS protein PSF2 |
| 209773_s_at | 17.73 | 7.37 | 2.40 | − | RRM2 | ribonucleotide reductase M2 polypeptide |
| 209540_at | 27.99 | 12.37 | 2.26 | + | IGF1 | insulin-like growth factor 1 (somatomedin C) |
| 213033_s_at | 24.87 | 11.15 | 2.23 | + | NFIB | Nuclear factor I/B |
| 213734_at | 5.51 | 2.52 | 2.18 | − | WSB2 | WD repeat and SOCS box-containing 2 |
| 204767_s_at | 7.16 | 3.28 | 2.18 | − | FEN1 | flap structure-specific endonuclease 1 |
| 204127_at | 3.68 | 1.82 | 2.02 | − | RFC3 | replication factor C (activator 1) 3, 38 kDa |
| 208752_x_at | 1.16 | 0.59 | 1.97 | + | NAP1L1 | nucleosome assembly protein 1-like 1 |

Oncogenesis

| | | | | | | |
|---|---|---|---|---|---|---|
| 208079_s_at | 83.78 | 19.84 | 4.22 | − | STK6 | serine/threonine kinase 6 |
| 204092_s_at | 43.30 | 11.83 | 3.66 | − | STK6 | serine/threonine kinase 6 |
| 213829_x_at | 6.41 | 2.42 | 2.65 | − | TNFRSF6B | tumor necrosis factor receptor superfamily, |
| | | | | | | member 6b, decoy |
| 206413_s_at | 36.36 | 14.96 | 2.43 | − | TCL1B | T-cell leukemia/lymphoma 1B |
| 203035_s_at | 7.62 | 3.14 | 2.42 | − | PIAS3 | protein inhibitor of activated STAT, 3 |
| 202095_s_at | 51.32 | 21.44 | 2.39 | − | BIRC5 | baculoviral IAP repeat-containing 5 (survivin) |
| 210434_x_at | 3.61 | 1.54 | 2.34 | − | JTB | jumping translocation breakpoint |
| 209054_s_at | 3.75 | 1.81 | 2.08 | − | WHSC1 | Wolf-Hirschhorn syndrome candidate 1 |
| 200048_s_at | 2.32 | 1.14 | 2.04 | − | JTB | jumping translocation breakpoint |
| 203554_x_at | 9.16 | 4.61 | 1.98 | − | PTTG1 | pituitary tumor-transforming 1 |
| 203192_at | 5.92 | 3.01 | 1.97 | − | ABCB6 | ATP-binding cassette, sub-family B (MDR/TAP), |
| | | | | | | member 6 |

Metabolism

| | | | | | | |
|---|---|---|---|---|---|---|
| 212070_at | 41.12 | 14.17 | 2.90 | − | GPR56 | G protein-coupled receptor 56 |
| 221256_s_at | 21.39 | 7.39 | 2.89 | + | HDHD3 | haloacid dehalogenase-like hydrolase domain |
| | | | | | | containing 3 |
| 203067_at | 13.34 | 4.66 | 2.86 | − | PDHX | pyruvate dehydrogenase complex, component X |
| 212062_at | 35.52 | 12.70 | 2.80 | − | ATP9A | ATPase, Class II, type 9A |
| 202651_at | 17.67 | 6.42 | 2.75 | − | LPGAT1 | lysophosphatidylglycerol acyltransferase 1 |
| 220892_s_at | 25.32 | 9.50 | 2.67 | + | PSAT1 | phosphoserine aminotransferase 1 |
| 206335_at | 9.17 | 3.62 | 2.53 | − | GALNS | galactosamine (N-acetyl)-6-sulfate sulfatase |
| 202722_s_at | 16.76 | 6.66 | 2.51 | − | GFPT1 | glutamine-fructose-6-phosphate transaminase 1 |
| 212353_at | 45.42 | 18.09 | 2.51 | − | SULF1 | sulfatase 1 |
| 221928_at | 39.21 | 16.23 | 2.42 | + | ACACB | acetyl-Coenzyme A carboxylase beta |
| 219616_at | 10.26 | 4.30 | 2.39 | − | FLJ21963 | FLJ21963 protein |
| 202464_s_at | 48.50 | 20.47 | 2.37 | − | PFKFB3 | 6-phosphofructo-2-kinase/fructose-2,6- |
| | | | | | | biphosphatase 3 |
| 59705_at | 9.15 | 3.93 | 2.33 | − | SCLY | selenocysteine lyase |
| 217776_at | 21.38 | 9.75 | 2.19 | − | RDH11 | retinol dehydrogenase 11 |
| 218025_s_at | 9.02 | 4.32 | 2.09 | + | PECI | peroxisomal D3,D2-enoyl-CoA isomerase |
| 209935_at | 12.20 | 5.92 | 2.06 | − | ATP2C1 | ATPase, Ca++ transporting, type 2C, member 1 |
| 200824_at | 31.66 | 15.69 | 2.02 | + | GSTP1 | glutathione S-transferase pi |
| 201626_at | 4.32 | 2.15 | 2.01 | − | INSIG1 | insulin induced gene 1 |

Cellular defense response

| | | | | | | |
|---|---|---|---|---|---|---|
| 215633_x_at | 13.89 | 3.94 | 3.52 | + | LST1 | leukocyte specific transcript 1 |
| 210629_x_at | 5.76 | 1.66 | 3.47 | + | LST1 | leukocyte specific transcript 1 |
| 206983_at | 12.57 | 3.83 | 3.28 | + | CCR6 | chemokine (C—C motif) receptor 6 |
| 211582_x_at | 13.68 | 4.48 | 3.06 | + | LST1 | leukocyte specific transcript 1 |
| 211581_x_at | 9.43 | 3.52 | 2.68 | + | LST1 | leukocyte specific transcript 1 |
| 210116_at | 21.00 | 8.06 | 2.61 | + | SH2D1A | SH2 domain protein 1A, Duncan's disease |
| 211529_x_at | 6.25 | 2.59 | 2.41 | + | HLA-G | HLA-G histocompatibility antigen, class I, G |
| 210514_x_at | 9.37 | 4.20 | 2.23 | + | HLA-G | HLA-G histocompatibility antigen, class I, G |
| 211528_x_at | 4.96 | 2.35 | 2.11 | + | HLA-G | HLA-G histocompatibility antigen, class I, G |
| 207008_at | 12.62 | 6.08 | 2.08 | + | IL8RB | interleukin 8 receptor, beta |
| 206978_at | 4.21 | 2.05 | 2.05 | + | CCR2 | chemokine (C—C motif) receptor 2 |
| 211567_at | 10.37 | 5.27 | 1.97 | + | — | — |
| 205495_s_at | 7.10 | 3.63 | 1.96 | + | GNLY | granulysin |

Chemotaxis

| | | | | | | |
|---|---|---|---|---|---|---|
| 206983_at | 15.76 | 4.80 | 3.28 | + | CCR6 | chemokine (C—C motif) receptor 6 |
| 210072_at | 30.51 | 10.90 | 2.80 | + | CCL19 | chemokine (C—C motif) ligand 19 |
| 207850_at | 17.47 | 6.65 | 2.63 | + | CXCL3 | chemokine (C—X—C motif) ligand 3 |
| 216598_s_at | 28.42 | 11.20 | 2.54 | + | CCL2 | chemokine (C—C motif) ligand 2 |
| 214435_x_at | 4.34 | 1.82 | 2.39 | − | RALA | v-ral simian leukemia viral oncogene homolog A |
| | | | | | | (ras related) |
| 204470_at | 7.69 | 3.23 | 2.38 | + | CXCL1 | chemokine (C—X—C motif) ligand 1 |
| 209687_at | 13.77 | 5.85 | 2.35 | + | CXCL12 | chemokine (C—X—C motif) ligand 12 (stromal cell- |
| | | | | | | derived factor 1) |
| 203666_at | 5.37 | 2.38 | 2.26 | + | CXCL12 | chemokine (C—X—C motif) ligand 12 (stromal cell- |
| | | | | | | derived factor 1) |
| 207008_at | 15.81 | 7.61 | 2.08 | + | IL8RB | interleukin 8 receptor, beta |
| 209201_x_at | 9.29 | 4.50 | 2.06 | + | CXCR4 | chemokine (C—X—C motif) receptor 4 |
| 206978_at | 5.28 | 2.57 | 2.05 | + | CCR2 | chemokine (C—C motif) receptor 2 |
| 206337_at | 6.09 | 3.06 | 1.99 | + | CCR7 | chemokine (C—C motif) receptor 7 |
| 211567_at | 13.00 | 6.60 | 1.97 | + | — | — |
| 214974_x_at | 8.80 | 4.49 | 1.96 | + | CXCL5 | chemokine (C—X—C motif) ligand 5 |

TABLE 6
significant genes in the top ten pathways for ER negative tumors

| | | | | | | |
|---|---|---|---|---|---|---|
| | | | | | Gene | |
| PSID | influence | sd | z-score | info | Symbol | Gene Title |

Regulation of cell growth

| | | | | | | |
|---|---|---|---|---|---|---|
| 209648_x_at | 23.16 | 5.77 | 4.01 | − | SOCS5 | suppressor of cytokine signaling 5 |
| 208127_s_at | 13.90 | 3.71 | 3.75 | − | SOCS5 | suppressor of cytokine signaling 5 |
| 209550_at | 18.66 | 5.88 | 3.18 | − | NDN | necdin homolog (mouse) |
| 201162_at | 16.18 | 5.15 | 3.14 | − | IGFBP7 | insulin-like growth factor binding protein 7 |
| 212279_at | 13.20 | 4.53 | 2.91 | + | MAC30 | hypothetical protein MAC30 |
| 213337_s_at | 7.30 | 2.53 | 2.88 | + | SOCS1 | suppressor of cytokine signaling 1 |
| 213910_at | 37.27 | 12.99 | 2.87 | − | IGFBP7 | insulin-like growth factor binding protein 7 |
| 217982_s_at | 3.33 | 1.20 | 2.78 | − | MORF4L1 | mortality factor 4 like 1 |
| 201185_at | 10.66 | 3.90 | 2.73 | − | HTRA1 | HtrA serine peptidase 1 |
| 209101_at | 18.31 | 6.81 | 2.69 | − | CTGF | connective tissue growth factor |
| 202149_at | 12.23 | 5.12 | 2.39 | − | NEDD9 | neural precursor cell expressed, |
| | | | | | | developmentally down-regulated 9 |
| 201163_s_at | 3.89 | 1.69 | 2.31 | − | IGFBP7 | insulin-like growth factor binding protein 7 |
| 208394_x_at | 4.40 | 2.07 | 2.12 | − | ESM1 | endothelial cell-specific molecule 1 |
| 211513_s_at | 23.97 | 11.32 | 2.12 | + | OGFR | opioid growth factor receptor |
| 211512_s_at | 4.18 | 2.11 | 1.98 | + | OGFR | opioid growth factor receptor |

Regulation of G-protein coupled receptor signaling pathway

| | | | | | | |
|---|---|---|---|---|---|---|
| 204337_at | 31.44 | 7.89 | 3.99 | − | RGS4 | regulator of G-protein signalling 4 |
| 209324_s_at | 10.18 | 2.73 | 3.73 | − | RGS16 | regulator of G-protein signalling 16 |
| 220300_at | 9.44 | 3.61 | 2.61 | − | RGS3 | regulator of G-protein signalling 3 |
| 202388_at | 24.64 | 9.45 | 2.61 | − | RGS2 | regulator of G-protein signalling 2, 24 kDa |
| 204396_s_at | 5.77 | 2.47 | 2.34 | − | GRK5 | G protein-coupled receptor kinase 5 |

Skeletal development

| | | | | | | |
|---|---|---|---|---|---|---|
| 217404_s_at | 199.74 | 50.77 | 3.93 | − | COL2A1 | collagen, type II, alpha 1 |
| 210135_s_at | 14.72 | 4.62 | 3.19 | − | SHOX2 | short stature homeobox 2 |
| 205941_s_at | 14.81 | 5.41 | 2.74 | − | COL10A1 | collagen, type X, alpha 1 |
| 201792_at | 8.36 | 3.08 | 2.72 | − | AEBP1 | AE binding protein 1 |
| 206091_at | 25.05 | 9.62 | 2.60 | − | MATN3 | matrilin 3 |
| 208443_x_at | 18.61 | 7.88 | 2.36 | − | SHOX2 | short stature homeobox 2 |
| 213943_at | 3.30 | 1.48 | 2.23 | − | TWIST1 | twist homolog 1(Drosophila) |
| 220076_at | 15.77 | 7.23 | 2.18 | − | ANKH | ankylosis, progressive homolog (mouse) |
| 210427_x_at | 1.45 | 0.69 | 2.10 | − | ANXA2 | annexin A2 |
| 210809_s_at | 3.36 | 1.64 | 2.05 | − | POSTN | periostin, osteoblast specific factor |
| 210973_s_at | 12.86 | 6.33 | 2.03 | + | FGFR1 | fibroblast growth factor receptor 1 |
| 213503_x_at | 1.24 | 0.64 | 1.96 | − | ANXA2 | annexin A2 |

Protein amino acid phosphorylation

| | | | | | | |
|---|---|---|---|---|---|---|
| 213595_s_at | 70.67 | 19.13 | 3.69 | − | CDC42BPA | CDC42 binding protein kinase alpha (DMPK- |
| | | | | | | like) |
| 215050_x_at | 47.49 | 13.74 | 3.46 | + | MAPKAPK2 | mitogen-activated protein kinase-activated |
| | | | | | | protein kinase 2 |
| 208875_s_at | 10.32 | 3.05 | 3.39 | + | PAK2 | p21 (CDKN1A)-activated kinase 2 |
| 216711_s_at | 12.50 | 3.71 | 3.37 | + | TAF1 | TAF1 RNA polymerase II, TATA box binding |
| | | | | | | protein (TBP)-associated factor |
| 203131_at | 24.32 | 7.64 | 3.18 | − | PDGFRA | platelet-derived growth factor receptor, alpha |
| | | | | | | polypeptide |
| 214683_s_at | 32.74 | 10.72 | 3.05 | − | CLK1 | CDC-like kinase 1 |
| 201401_s_at | 103.31 | 33.85 | 3.05 | + | ADRBK1 | adrenergic, beta, receptor kinase 1 |
| 203552_at | 12.54 | 4.52 | 2.77 | − | MAP4K5 | mitogen-activated protein kinase kinase |
| | | | | | | kinase kinase 5 |
| 205880_at | 6.18 | 2.31 | 2.68 | − | PRKD1 | protein kinase D1 |
| 200604_s_at | 20.81 | 8.27 | 2.52 | + | PRKAR1A | protein kinase, cAMP-dependent, regulatory, |
| | | | | | | type I, alpha |
| 207239_s_at | 19.06 | 7.73 | 2.47 | + | PCTK1 | PCTAIRE protein kinase 1 |
| 214007_s_at | 60.27 | 24.46 | 2.46 | + | PTK9 | PTK9 protein tyrosine kinase 9 |
| 212530_at | 8.39 | 3.43 | 2.45 | − | NEK7 | NIMA (never in mitosis gene a)-related kinase 7 |
| 212740_at | 5.21 | 2.15 | 2.43 | − | PIK3R4 | phosphoinositide-3-kinase, regulatory subunit |
| | | | | | | 4, p150 |
| 215296_at | 42.64 | 17.82 | 2.39 | − | CDC42BPA | CDC42 binding protein kinase alpha (DMPK- |
| | | | | | | like) |
| 201461_s_at | 20.08 | 8.57 | 2.34 | + | MAPKAPK2 | mitogen-activated protein kinase-activated |
| | | | | | | protein kinase 2 |
| 204396_s_at | 13.51 | 5.78 | 2.34 | − | GRK5 | G protein-coupled receptor kinase 5 |
| 207667_s_at | 14.58 | 6.35 | 2.30 | + | MAP2K3 | mitogen-activated protein kinase kinase 3 |
| 202127_at | 10.85 | 4.86 | 2.23 | − | PRPF4B | PRP4 pre-mRNA processing factor 4 homolog |
| | | | | | | B (yeast) |
| 59644_at | 9.95 | 4.50 | 2.21 | − | BMP2K | BMP2 inducible kinase |
| 207228_at | 15.38 | 6.96 | 2.21 | + | PRKACG | protein kinase, cAMP-dependent, catalytic, |
| | | | | | | gamma |
| 213490_s_at | 43.56 | 20.23 | 2.15 | + | MAP2K2 | mitogen-activated protein kinase kinase 2 |
| 211599_x_at | 8.19 | 3.83 | 2.14 | + | MET | met proto-oncogene (hepatocyte growth factor |
| | | | | | | receptor) |
| 211208_s_at | 7.35 | 3.44 | 2.14 | + | CASK | calcium/calmodulin-dependent serine protein |
| | | | | | | kinase (MAGUK family) |
| 205578_at | 20.67 | 9.69 | 2.13 | − | ROR2 | receptor tyrosine kinase-like orphan receptor 2 |
| 204813_at | 6.64 | 3.30 | 2.01 | + | MAPK10 | mitogen-activated protein kinase 10 |
| 208824_x_at | 12.76 | 6.35 | 2.01 | + | PCTK1 | PCTAIRE protein kinase 1 |

Cell adhesion

| | | | | | | |
|---|---|---|---|---|---|---|
| 212724_at | 22.05 | 6.48 | 3.40 | − | RND3 | Rho family GTPase 3 |
| 209210_s_at | 26.72 | 8.13 | 3.28 | − | PLEKHC1 | pleckstrin homology domain containing, family |
| | | | | | | C member 1 |
| 202363_at | 24.96 | 7.95 | 3.14 | − | SPOCK | sparc/osteonectin, cwcv and kazal-like |
| | | | | | | domains proteoglycan (testican) |
| 209651_at | 15.39 | 4.94 | 3.12 | − | TGFB1I1 | transforming growth factor beta 1 induced |
| | | | | | | transcript 1 |
| 201505_at | 21.00 | 7.24 | 2.90 | − | LAMB1 | laminin, beta 1 |
| 200771_at | 8.56 | 3.01 | 2.84 | − | LAMC1 | laminin, gamma 1 (formerly LAMB2) |
| 213790_at | 14.02 | 4.96 | 2.83 | − | ADAM12 | ADAM metallopeptidase domain 12 (meltrin |
| | | | | | | alpha) |
| 203083_at | 12.25 | 4.39 | 2.79 | − | THBS2 | thrombospondin 2 |
| 222020_s_at | 62.24 | 22.64 | 2.75 | − | HNT | neurotrimin |
| 205532_s_at | 42.40 | 15.54 | 2.73 | + | CDH6 | cadherin 6, type 2, K-cadherin (fetal kidney) |
| 201792_at | 18.97 | 6.98 | 2.72 | − | AEBP1 | AE binding protein 1 |
| 209101_at | 19.18 | 7.13 | 2.69 | − | CTGF | connective tissue growth factor |
| 215904_at | 29.42 | 11.01 | 2.67 | + | MLLT4 | myeloid/lymphoid or mixed-lineage leukemia |
| | | | | | | (trithorax homolog, Drosophila); translocated |
| | | | | | | to, 4 |
| 201561_s_at | 6.71 | 2.62 | 2.56 | + | CLSTN1 | calsyntenin 1 |
| 204677_at | 11.48 | 4.53 | 2.53 | − | CDH5 | cadherin 5, type 2, VE-cadherin (vascular |
| | | | | | | epithelium) |
| 214212_x_at | 10.68 | 4.26 | 2.51 | − | PLEKHC1 | pleckstrin homology domain containing, family |
| | | | | | | C (with FERM domain) member 1 |
| 214375_at | 23.91 | 10.02 | 2.39 | − | PPFIBP1 | PTPRF interacting protein, binding protein 1 |
| | | | | | | (liprin beta 1) |
| 202149_at | 12.81 | 5.37 | 2.39 | − | NEDD9 | neural precursor cell expressed, |
| | | | | | | developmentally down-regulated 9 |
| 204955_at | 12.74 | 5.34 | 2.39 | − | SRPX | sushi-repeat-containing protein, X-linked |
| 209873_s_at | 11.75 | 5.14 | 2.29 | + | PKP3 | plakophilin 3 |
| 211208_s_at | 5.66 | 2.65 | 2.14 | + | CASK | calcium/calmodulin-dependent serine protein |
| | | | | | | kinase (MAGUK family) |
| 205176_s_at | 3.87 | 1.82 | 2.13 | − | ITGB3BP | integrin beta 3 binding protein (beta3- |
| | | | | | | endonexin) |
| 201281_at | 2.86 | 1.39 | 2.06 | + | ADRM1 | adhesion regulating molecule 1 |
| 212843_at | 22.00 | 10.69 | 2.06 | − | NCAM1 | neural cell adhesion molecule 1 |
| 210809_s_at | 7.63 | 3.72 | 2.05 | − | POSTN | periostin, osteoblast specific factor |
| 205656_at | 4.03 | 1.96 | 2.05 | − | PCDH17 | protocadherin 17 |
| 201438_at | 5.86 | 2.89 | 2.03 | − | COL6A3 | collagen, type VI, alpha 3 |
| 213241_at | 6.19 | 3.06 | 2.02 | − | PLXNC1 | plexin C1 |
| 218975_at | 26.96 | 13.55 | 1.99 | − | COL5A3 | collagen, type V, alpha 3 |

Carbohydrate metabolism

| | | | | | | |
|---|---|---|---|---|---|---|
| 202499_s_at | 39.16 | 13.68 | 2.86 | − | SLC2A3 | solute carrier family 2 (facilitated glucose |
| | | | | | | transporter), member 3 |
| 216010_x_at | 91.48 | 32.31 | 2.83 | + | FUT3 | fucosyltransferase 3 |
| 205799_s_at | 17.32 | 6.72 | 2.58 | + | SLC3A1 | solute carrier family 3, member 1 |
| 201765_s_at | 4.24 | 2.08 | 2.04 | + | HEXA | hexosaminidase A (alpha polypeptide) |

Nuclear mRNA splicing, via splicesome

| | | | | | | |
|---|---|---|---|---|---|---|
| 200686_s_at | 20.80 | 5.76 | 3.61 | − | SFRS11 | splicing factor, arginine/serine-rich 11 |
| 203376_at | 7.88 | 2.58 | 3.06 | − | CDC40 | cell division cycle 40 homolog (yeast) |
| 209162_s_at | 45.77 | 16.98 | 2.69 | + | PRPF4 | PRP4 pre-mRNA processing factor 4 homolog |
| | | | | | | (yeast) |
| 201698_s_at | 3.64 | 1.44 | 2.52 | + | SFRS9 | splicing factor, arginine/serine-rich 9 |
| 200685_at | 17.74 | 7.38 | 2.40 | − | SFRS11 | splicing factor, arginine/serine-rich 11 |
| 202127_at | 10.16 | 4.55 | 2.23 | − | PRPF4B | PRP4 pre-mRNA processing factor 4 homolog |
| | | | | | | B (yeast) |
| 221546_at | 31.79 | 14.83 | 2.14 | + | PRPF18 | PRP18 pre-mRNA processing factor 18 |
| | | | | | | homolog (yeast) |
| 201385_at | 3.45 | 1.66 | 2.08 | − | DHX15 | DEAH (Asp-Glu-Ala-His) box polypeptide 15 |
| 204064_at | 7.66 | 3.76 | 2.04 | − | THOC1 | THO complex 1 |
| 214016_s_at | 8.09 | 4.04 | 2.00 | − | SFPQ | Splicing factor proline/glutamine-rich |
| 219119_at | 3.44 | 1.75 | 1.97 | − | LSM8 | LSM8 homolog, U6 small nuclear RNA |
| | | | | | | associated |

Signal transduction

| | | | | | | |
|---|---|---|---|---|---|---|
| 204337_at | 77.97 | 19.56 | 3.99 | − | RGS4 | regulator of G-protein signalling 4 |
| 209324_s_at | 25.24 | 6.77 | 3.73 | − | RGS16 | regulator of G-protein signalling 16 |
| 204464_s_at | 14.07 | 3.89 | 3.62 | − | EDNRA | endothelin receptor type A |
| 202247_s_at | 14.76 | 4.24 | 3.48 | + | MTA1 | metastasis associated 1 |
| 221773_at | 16.08 | 4.70 | 3.42 | − | ELK3 | ELK3, ETS-domain protein (SRF accessory |
| | | | | | | protein 2) |
| 203328_x_at | 3.87 | 1.13 | 3.41 | + | IDE | insulin-degrading enzyme |
| 208875_s_at | 10.94 | 3.23 | 3.39 | + | PAK2 | p21 (CDKN1A)-activated kinase 2 |
| 201835_s_at | 19.43 | 6.22 | 3.12 | + | PRKAB1 | protein kinase, AMP-activated, beta 1 non- |
| | | | | | | catalytic subunit |
| 217496_s_at | 6.53 | 2.13 | 3.07 | + | IDE | insulin-degrading enzyme |
| 209895_at | 64.80 | 21.23 | 3.05 | + | PTPN11 | protein tyrosine phosphatase, non-receptor |
| | | | | | | type 11 |
| 201401_s_at | 109.49 | 35.88 | 3.05 | + | ADRBK1 | adrenergic, beta, receptor kinase 1 |
| 202716_at | 7.60 | 2.50 | 3.05 | + | PTPN1 | protein tyrosine phosphatase, non-receptor |
| | | | | | | type 1 |
| 215984_s_at | 129.29 | 44.77 | 2.89 | + | ARFRP1 | ADP-ribosylation factor related protein 1 |
| 219837_s_at | 84.68 | 29.97 | 2.83 | − | CYTL1 | cytokine-like 1 |
| 207987_s_at | 96.20 | 34.37 | 2.80 | − | GNRH1 | gonadotropin-releasing hormone 1 |
| 204115_at | 15.78 | 5.64 | 2.80 | − | GNG11 | guanine nucleotide binding protein (G |
| | | | | | | protein), gamma 11 |
| 218157_x_at | 13.07 | 4.70 | 2.78 | + | CDC42SE1 | CDC42 small effector 1 |
| 211302_s_at | 34.25 | 12.62 | 2.71 | + | PDE4B | phosphodiesterase 4B, cAMP-specific |
| 215904_at | 40.46 | 15.15 | 2.67 | + | MLLT4 | myeloid/lymphoid or mixed-lineage leukemia; |
| | | | | | | translocated to, 4 |
| 205701_at | 32.40 | 12.37 | 2.62 | + | IPO8 | importin 8 |
| 202388_at | 61.10 | 23.45 | 2.61 | − | RGS2 | regulator of G-protein signalling 2, 24 kDa |
| 213446_s_at | 17.87 | 6.86 | 2.60 | + | IQGAP1 | IQ motif containing GTPase activating protein 1 |
| 222201_s_at | 23.74 | 9.21 | 2.58 | − | CASP8AP2 | CASP8 associated protein 2 |
| 201065_s_at | 8.99 | 3.55 | 2.53 | + | GTF2I | general transcription factor II, I |
| 35150_at | 7.62 | 3.06 | 2.49 | + | CD40 | CD40 antigen (TNF receptor superfamily |
| | | | | | | member 5) |
| 212294_at | 10.32 | 4.16 | 2.48 | − | GNG12 | guanine nucleotide binding protein (G |
| | | | | | | protein), gamma 12 |
| 200644_at | 9.85 | 4.00 | 2.46 | + | MARCKSL1 | MARCKS-like 1 |
| 210221_at | 14.37 | 5.85 | 2.46 | + | CHRNA3 | cholinergic receptor, nicotinic, alpha |
| | | | | | | polypeptide 3 |
| 211245_x_at | 28.38 | 11.62 | 2.44 | + | KIR2DL4 | killer cell immunoglobulin-like receptor, two |
| | | | | | | domains, long cytoplasmic tail, 4 |
| 211242_x_at | 78.57 | 32.17 | 2.44 | + | KIR2DL4 | killer cell immunoglobulin-like receptor, two |
| | | | | | | domains, long cytoplasmic tail, 4 |
| 221386_at | 17.71 | 7.29 | 2.43 | + | OR3A2 | olfactory receptor, family 3, subfamily A, |
| | | | | | | member 2 |
| 202149_at | 17.62 | 7.38 | 2.39 | − | NEDD9 | neural precursor cell expressed, |
| | | | | | | developmentally down-regulated 9 |
| 201008_s_at | 50.83 | 21.32 | 2.38 | + | TXNIP | thioredoxin interacting protein |
| 202467_s_at | 6.12 | 2.57 | 2.38 | − | COPS2 | COP9 constitutive photomorphogenic |
| | | | | | | homolog subunit 2 (Arabidopsis) |
| 204396_s_at | 14.32 | 6.12 | 2.34 | − | GRK5 | G protein-coupled receptor kinase 5 |
| 396_f_at | 9.39 | 4.05 | 2.32 | + | EPOR | erythropoietin receptor |
| 201488_x_at | 2.09 | 0.91 | 2.31 | + | KHDRBS1 | KH domain containing, RNA binding, signal |
| | | | | | | transduction associated 1 |
| 221745_at | 17.06 | 7.42 | 2.30 | + | WDR68 | WD repeat domain 68 |
| 207667_s_at | 15.45 | 6.73 | 2.30 | + | MAP2K3 | mitogen-activated protein kinase kinase 3 |
| 209505_at | 73.82 | 32.44 | 2.28 | − | NR2F1 | Nuclear receptor subfamily 2, group F, |
| | | | | | | member 1 |
| 213401_s_at | 76.88 | 33.94 | 2.27 | − | — | — |
| 202091_at | 16.37 | 7.23 | 2.26 | + | ARL2BP | ADP-ribosylation factor-like 2 binding protein |
| 201009_s_at | 25.86 | 11.52 | 2.25 | + | TXNIP | thioredoxin interacting protein |
| 213270_at | 5.27 | 2.36 | 2.24 | + | MPP2 | membrane protein, palmitoylated 2 (MAGUK |
| | | | | | | p55 subfamily member 2) |
| 209239_at | 4.89 | 2.27 | 2.15 | + | NFKB1 | nuclear factor of kappa light polypeptide gene |
| | | | | | | enhancer in B-cells 1 (p105) |
| 211599_x_at | 8.68 | 4.06 | 2.14 | + | MET | met proto-oncogene (hepatocyte growth factor |
| | | | | | | receptor) |
| 205578_at | 21.90 | 10.27 | 2.13 | − | ROR2 | receptor tyrosine kinase-like orphan receptor 2 |
| 205176_s_at | 5.32 | 2.50 | 2.13 | − | ITGB3BP | integrin beta 3 binding protein (beta3- |
| | | | | | | endonexin) |
| 206132_at | 1.84 | 0.87 | 2.11 | + | MCC | mutated in colorectal cancers |
| 203218_at | 22.38 | 10.69 | 2.09 | − | MAPK9 | mitogen-activated protein kinase 9 |
| 33814_at | 10.79 | 5.17 | 2.09 | + | PAK4 | p21(CDKN1A)-activated kinase 4 |
| 203077_s_at | 5.06 | 2.43 | 2.08 | − | SMAD2 | SMAD, mothers against DPP homolog 2 |
| | | | | | | (Drosophila) |
| 201431_s_at | 9.40 | 4.52 | 2.08 | − | DPYSL3 | dihydropyrimidinase-like 3 |
| 221060_s_at | 14.80 | 7.12 | 2.08 | + | TLR4 | toll-like receptor 4 |
| 204712_at | 58.79 | 28.53 | 2.06 | − | WIF1 | WNT inhibitory factor 1 |
| 200923_at | 21.83 | 10.68 | 2.04 | + | LGALS3BP | lectin, galactoside-binding, soluble, 3 binding |
| | | | | | | protein |
| 204064_at | 8.66 | 4.25 | 2.04 | − | THOC1 | THO complex 1 |
| 218158_s_at | 8.68 | 4.29 | 2.02 | − | APPL | adaptor protein containing pH domain, PTB |
| | | | | | | domain and leucine zipper motif 1 |
| 204813_at | 7.04 | 3.50 | 2.01 | + | MAPK10 | mitogen-activated protein kinase 10 |
| 208486_at | 3.82 | 1.91 | 2.00 | + | DRD5 | dopamine receptor D5 |

Cation transport

| | | | | | | |
|---|---|---|---|---|---|---|
| 205802_at | 76.09 | 17.70 | 4.30 | − | TRPC1 | transient receptor potential cation channel, |
| | | | | | | subfamily C, member 1 |
| 203688_at | 16.25 | 4.21 | 3.86 | − | PKD2 | polycystic kidney disease 2 (autosomal |
| | | | | | | dominant) |
| 205803_s_at | 21.92 | 6.71 | 3.26 | − | TRPC1 | transient receptor potential cation channel, |
| | | | | | | subfamily C, member 1 |
| 212297_at | 4.78 | 1.92 | 2.49 | − | ATP13A3 | ATPase type 13A3 |
| 208349_at | 5.70 | 2.33 | 2.45 | + | TRPA1 | transient receptor potential cation channel, |
| | | | | | | subfamily A, member 1 |

Calcium ion transport

| | | | | | | |
|---|---|---|---|---|---|---|
| 205802_at | 60.75 | 14.13 | 4.30 | − | TRPC1 | transient receptor potential cation channel, |
| | | | | | | subfamily C, member 1 |
| 205803_s_at | 17.50 | 5.36 | 3.26 | − | TRPC1 | transient receptor potential cation channel, |
| | | | | | | subfamily C, member 1 |
| 219090_at | 32.29 | 13.55 | 2.38 | − | SLC24A3 | solute carrier family 24 |
| | | | | | | (sodium/potassium/calcium exchanger), |
| | | | | | | member 3 |

Protein modification

| | | | | | | |
|---|---|---|---|---|---|---|
| 220483_s_at | 131.49 | 33.34 | 3.94 | + | RNF19 | ring finger protein 19 |
| 205571_at | 16.80 | 4.32 | 3.89 | − | LIPT1 | lipoyltransferase 1 |
| 208689_s_at | 13.18 | 4.81 | 2.74 | + | RPN2 | ribophorin II |
| 213704_at | 12.56 | 5.11 | 2.46 | − | RABGGTB | Rab geranylgeranyltransferase, beta subunit |

Intracellular signaling cascade

| | | | | | | |
|---|---|---|---|---|---|---|
| 209648_x_at | 35.05 | 8.74 | 4.01 | − | SOCS5 | suppressor of cytokine signaling 5 |
| 208127_s_at | 21.05 | 5.61 | 3.75 | − | SOCS5 | suppressor of cytokine signaling 5 |
| 219165_at | 14.50 | 4.12 | 3.52 | − | PDLIM2 | PDZ and LIM domain 2 (mystique) |
| 212729_at | 13.42 | 3.94 | 3.41 | + | DLG3 | discs, large homolog 3 (neuroendocrine-dlg, |
| | | | | | | Drosophila) |
| 221748_s_at | 17.17 | 5.23 | 3.28 | − | TNS1 | tensin 1 |
| 215829_at | 13.31 | 4.23 | 3.15 | + | SHANK2 | SH3 and multiple ankyrin repeat domains 2 |
| 209895_at | 68.09 | 22.31 | 3.05 | + | PTPN11 | protein tyrosine phosphatase, non-receptor |
| | | | | | | type 11 |
| 212801_at | 5.40 | 1.77 | 3.04 | + | CIT | citron (rho-interacting, serine/threonine kinase |
| | | | | | | 21) |
| 202226_s_at | 55.90 | 18.78 | 2.98 | + | CRK | v-crk sarcoma virus CT10 oncogene homolog |
| | | | | | | (avian) |
| 213337_s_at | 11.05 | 3.83 | 2.88 | + | SOCS1 | suppressor of cytokine signaling 1 |
| 209684_at | 5.91 | 2.06 | 2.87 | − | RIN2 | Ras and Rab interactor 2 |
| 207732_s_at | 17.40 | 6.20 | 2.81 | + | DLG3 | discs, large homolog 3 (neuroendocrine-dlg, |
| | | | | | | Drosophila) |
| 203370_s_at | 30.18 | 11.04 | 2.73 | − | PDLIM7 | PDZ and LIM domain 7 (enigma) |
| 213545_x_at | 12.62 | 4.65 | 2.71 | − | SNX3 | sorting nexin 3 |
| 205880_at | 6.88 | 2.57 | 2.68 | − | PRKD1 | protein kinase D1 |
| 210648_x_at | 10.35 | 3.91 | 2.65 | − | SNX3 | sorting nexin 3 |
| 202114_at | 10.97 | 4.15 | 2.64 | − | SNX2 | sorting nexin 2 |
| 218705_s_at | 22.90 | 8.73 | 2.62 | − | SNX24 | sorting nexing 24 |
| 220300_at | 24.59 | 9.42 | 2.61 | − | RGS3 | regulator of G-protein signalling 3 |
| 205147_x_at | 5.11 | 2.01 | 2.54 | + | NCF4 | neutrophil cytosolic factor 4, 40 kDa |
| 207782_s_at | 25.02 | 9.94 | 2.52 | + | PSEN1 | presenilin 1 |
| 200604_s_at | 23.18 | 9.21 | 2.52 | + | PRKAR1A | protein kinase, cAMP-dependent, regulatory, |
| | | | | | | type I, alpha |
| 200067_x_at | 7.46 | 3.22 | 2.32 | − | SNX3 | sorting nexin 3 |
| 207105_s_at | 5.09 | 2.20 | 2.32 | + | PIK3R2 | phosphoinositide-3-kinase, regulatory subunit |
| | | | | | | 2 (p85 beta) |
| 205170_at | 9.41 | 4.22 | 2.23 | + | STAT2 | signal transducer and activator of transcription |
| | | | | | | 2, 113 kDa |
| 215411_s_at | 23.50 | 10.69 | 2.20 | − | TRAF3IP2 | TRAF3 interacting protein 2 |
| 219457_s_at | 15.25 | 7.45 | 2.05 | − | RIN3 | Ras and Rab interactor 3 |
| 221526_x_at | 12.87 | 6.32 | 2.04 | + | PARD3 | par-3 partitioning defective 3 homolog (C. elegans) |
| 209154_at | 3.29 | 1.66 | 1.98 | − | TAX1BP3 | Tax1 binding protein 3 |
| 202987_at | 19.16 | 9.79 | 1.96 | − | TRAF3IP2 | TRAF3 interacting protein 2 |

mRNA processing

| | | | | | | |
|---|---|---|---|---|---|---|
| 222040_at | 36.12 | 11.14 | 3.24 | − | HNRPA1 | heterogeneous nuclear ribonucleoprotein A1 |
| 208765_s_at | 21.68 | 6.81 | 3.18 | + | HNRPR | heterogeneous nuclear ribonucleoprotein R |
| 221919_at | 28.33 | 9.18 | 3.09 | − | — | — |
| 205063_at | 23.40 | 7.98 | 2.93 | − | SIP1 | survival of motor neuron protein interacting |
| | | | | | | protein 1 |
| 201488_x_at | 2.29 | 0.99 | 2.31 | + | KHDRBS1 | KH domain containing, RNA binding, signal |
| | | | | | | transduction associated 1 |
| 201224_s_at | 10.50 | 4.62 | 2.27 | + | SRRM1 | serine/arginine repetitive matrix 1 |

RNA splicing

| | | | | | | |
|---|---|---|---|---|---|---|
| 200686_s_at | 20.70 | 5.73 | 3.61 | − | SFRS11 | splicing factor, arginine/serine-rich 11 |
| 203376_at | 7.85 | 2.56 | 3.06 | − | CDC40 | cell division cycle 40 homolog (yeast) |
| 209162_s_at | 45.56 | 16.91 | 2.69 | + | PRPF4 | PRP4 pre-mRNA processing factor 4 homolog |
| | | | | | | (yeast) |
| 200685_at | 17.66 | 7.35 | 2.40 | − | SFRS11 | splicing factor, arginine/serine-rich 11 |
| 201362_at | 9.18 | 4.04 | 2.27 | − | IVNS1ABP | influenza virus NS1A binding protein |
| 202127_at | 10.12 | 4.53 | 2.23 | − | PRPF4B | PRP4 pre-mRNA processing factor 4 homolog |
| | | | | | | B (yeast) |
| 221546_at | 31.65 | 14.76 | 2.14 | + | PRPF18 | PRP18 pre-mRNA processing factor 18 |
| | | | | | | homolog (yeast) |
| 214016_s_at | 8.05 | 4.02 | 2.00 | − | SFPQ | Splicing factor proline/glutamine-rich |

Endotosis

| | | | | | | |
|---|---|---|---|---|---|---|
| 209839_at | 37.68 | 6.99 | 5.39 | − | DNM3 | dynamin 3 |
| 209684_at | 3.32 | 1.16 | 2.87 | − | RIN2 | Ras and Rab interactor 2 |
| 213545_x_at | 7.08 | 2.61 | 2.71 | − | SNX3 | sorting nexin 3 |
| 210648_x_at | 5.81 | 2.20 | 2.65 | − | SNX3 | sorting nexin 3 |
| 202114_at | 6.16 | 2.33 | 2.64 | − | SNX2 | sorting nexin 2 |
| 200067_x_at | 4.19 | 1.81 | 2.32 | − | SNX3 | sorting nexin 3 |
| 207287_at | 7.81 | 3.74 | 2.09 | − | FLJ14107 | hypothetical protein FLJ14107 |
| 219457_s_at | 8.56 | 4.18 | 2.05 | − | RIN3 | Ras and Rab interactor 3 |

Regulation of transcription from PolII promoter

| | | | | | | |
|---|---|---|---|---|---|---|
| 219778_at | 58.94 | 14.41 | 4.09 | − | ZFPM2 | zinc finger protein, multitype 2 |
| 221773_at | 13.43 | 3.93 | 3.42 | − | ELK3 | ELK3, ETS-domain protein (SRF accessory |
| | | | | | | protein 2) |
| 211251_x_at | 11.18 | 3.69 | 3.03 | + | NFYC | nuclear transcription factor Y, gamma |
| 202724_s_at | 9.60 | 3.34 | 2.88 | − | FOXO1A | forkhead box O1A |
| 212257_s_at | 14.37 | 5.13 | 2.80 | + | SMARCA2 | SWI/SNF related, matrix associated, actin |
| | | | | | | dependent regulator of chromatin, subfamily |
| | | | | | | a, member 2 |
| 202216_x_at | 9.15 | 3.28 | 2.79 | + | NFYC | nuclear transcription factor Y, gamma |
| 204349_at | 9.97 | 3.90 | 2.56 | − | CRSP9 | cofactor required for Sp1 transcriptional |
| | | | | | | activation, subunit 9, 33 kDa |
| 200604_s_at | 18.43 | 7.33 | 2.52 | + | PRKAR1A | protein kinase, cAMP-dependent, regulatory, |
| | | | | | | type I, alpha |
| 206858_s_at | 13.06 | 5.74 | 2.28 | − | HOXC6 | homeo box C6 |
| 205170_at | 7.49 | 3.35 | 2.23 | + | STAT2 | signal transducer and activator of transcription |
| | | | | | | 2, 113 kDa |
| 213891_s_at | 11.07 | 4.97 | 2.23 | − | TCF4 | Transcription factor 4 |
| 201073_s_at | 9.51 | 4.49 | 2.12 | + | SMARCC1 | SWI/SNF related, matrix associated, actin |
| | | | | | | dependent regulator of chromatin, subfamily |
| | | | | | | c, member 1 |
| 213251_at | 2.17 | 1.07 | 2.03 | − | SMARCA5 | SWI/SNF related, matrix associated, actin |
| | | | | | | dependent regulator of chromatin, subfamily |
| | | | | | | a, member 5 |
| 209292_at | 21.21 | 10.46 | 2.03 | − | ID4 | Inhibitor of DNA binding 4, dominant negative |
| | | | | | | helix-loop-helix protein |
| 209189_at | 61.47 | 30.61 | 2.01 | − | FOS | v-fos FBJ murine osteosarcoma viral |
| | | | | | | oncogene homolog |
| 202172_at | 6.04 | 3.07 | 1.97 | − | ZNF161 | zinc finger protein 161 |

Regulation of cell cycle

| | | | | | | |
|---|---|---|---|---|---|---|
| 216061_x_at | 7.05 | 2.09 | 3.38 | − | PDGFB | platelet-derived growth factor beta polypeptide |
| 209550_at | 23.27 | 7.33 | 3.18 | − | NDN | necdin homolog (mouse) |
| 214683_s_at | 30.04 | 9.83 | 3.05 | − | CLK1 | CDC-like kinase 1 |
| 211251_x_at | 11.58 | 3.82 | 3.03 | + | NFYC | nuclear transcription factor Y, gamma |
| 202216_x_at | 9.48 | 3.40 | 2.79 | + | NFYC | nuclear transcription factor Y, gamma |
| 205106_at | 47.82 | 17.22 | 2.78 | + | MTCP1 | mature T-cell proliferation 1 |
| 219910_at | 4.96 | 1.83 | 2.71 | + | HYPE | Huntingtin interacting protein E |
| 207239_s_at | 17.48 | 7.09 | 2.47 | + | PCTK1 | PCTAIRE protein kinase 1 |
| 202149_at | 15.25 | 6.39 | 2.39 | − | NEDD9 | neural precursor cell expressed, |
| | | | | | | developmentally down-regulated 9 |
| 38707_r_at | 1.72 | 0.80 | 2.16 | + | E2F4 | E2F transcription factor 4, p107/p130-binding |
| 204566_at | 6.86 | 3.21 | 2.14 | − | PPM1D | protein phosphatase 1D magnesium- |
| | | | | | | dependent, delta isoform |
| 201700_at | 5.14 | 2.44 | 2.11 | + | CCND3 | cyclin D3 |
| 200712_s_at | 5.65 | 2.72 | 2.07 | + | MAPRE1 | microtubule-associated protein, RP/EB family, |
| | | | | | | member 1 |
| 206272_at | 3.58 | 1.78 | 2.02 | − | SPHAR | S-phase response (cyclin-related) |
| 208824_x_at | 11.71 | 5.83 | 2.01 | + | PCTK1 | PCTAIRE protein kinase 1 |
| 2028_s_at | 1.07 | 0.55 | 1.95 | + | E2F1 | E2F transcription factor 1 |

Protein complex assembly

| | | | | | | |
|---|---|---|---|---|---|---|
| 212511_at | 7.99 | 2.34 | 3.41 | − | PICALM | phosphatidylinositol binding clathrin assembly |
| | | | | | | protein |
| 216711_s_at | 10.27 | 3.05 | 3.37 | + | TAF1 | TATA box binding protein (TBP)-associated |
| | | | | | | factor |
| 200771_at | 9.13 | 3.21 | 2.84 | − | LAMC1 | laminin, gamma 1 (formerly LAMB2) |
| 201624_at | 11.70 | 4.68 | 2.50 | − | DARS | aspartyl-tRNA synthetase |
| 35150_at | 5.91 | 2.37 | 2.49 | + | CD40 | CD40 antigen (TNF receptor superfamily |
| | | | | | | member 5) |
| 213480_at | 2.70 | 1.11 | 2.44 | − | VAMP4 | vesicle-associated membrane protein 4 |
| 213270_at | 4.09 | 1.83 | 2.24 | + | MPP2 | membrane protein, palmitoylated 2 (MAGUK |
| | | | | | | p55 subfamily member 2) |
| 208829_at | 8.14 | 3.73 | 2.18 | + | TAPBP | TAP binding protein (tapasin) |
| 216125_s_at | 13.70 | 6.39 | 2.15 | + | RANBP9 | RAN binding protein 9 |
| 212128_s_at | 12.43 | 5.88 | 2.11 | + | DAG1 | dystroglycan 1 (dystrophin-associated |
| | | | | | | glycoprotein 1) |
| 200841_s_at | 41.38 | 20.07 | 2.06 | + | EPRS | glutamyl-prolyl-tRNA synthetase |
| 221526_x_at | 9.49 | 4.67 | 2.04 | + | PARD3 | par-3 partitioning defective 3 homolog (C. elegans) |

Protein biosynthesis

| | | | | | | |
|---|---|---|---|---|---|---|
| 218830_at | 23.85 | 6.25 | 3.82 | − | RPL26L1 | ribosomal protein L26-like 1 |
| 202247_s_at | 24.00 | 6.89 | 3.48 | + | MTA1 | metastasis associated 1 |
| 214317_x_at | 21.82 | 7.39 | 2.95 | − | RPS9 | Ribosomal protein S9 |
| 200026_at | 5.33 | 1.91 | 2.78 | − | RPL34 | ribosomal protein L34 |
| 200963_x_at | 4.64 | 1.76 | 2.63 | − | RPL31 | ribosomal protein L31 |
| 221693_s_at | 25.44 | 9.85 | 2.58 | + | MRPS18A | mitochondrial ribosomal protein S18A |
| 219762_s_at | 15.45 | 6.27 | 2.46 | − | RPL36 | ribosomal protein L36 |
| 221593_s_at | 22.43 | 9.34 | 2.40 | − | RPL31 | ribosomal protein L31 |
| 200091_s_at | 3.20 | 1.36 | 2.35 | − | RPS25 | ribosomal protein S25 |
| 208756_at | 9.21 | 4.09 | 2.25 | + | EIF3S2 | eukaryotic translation initiation factor 3, |
| | | | | | | subunit 2 beta, 36 kDa |
| 203781_at | 9.61 | 4.31 | 2.23 | − | MRPL33 | mitochondrial ribosomal protein L33 |
| 202926_at | 9.86 | 4.58 | 2.15 | + | NAG | neuroblastoma-amplified protein |
| 213687_s_at | 6.78 | 3.19 | 2.13 | − | RPL35A | ribosomal protein L35a |
| 212450_at | 11.03 | 5.32 | 2.07 | − | KIAA0256 | KIAA0256 gene product |
| 214143_x_at | 4.08 | 2.08 | 1.96 | − | RPL24 | ribosomal protein L24 |

Cell cycle

| | | | | | | |
|---|---|---|---|---|---|---|
| 216711_s_at | 14.05 | 4.17 | 3.37 | + | TAF1 | TATA box binding protein (TBP)-associated |
| | | | | | | factor |
| 215747_s_at | 17.66 | 5.57 | 3.17 | + | RCC1 | regulator of chromosome condensation 1 |
| 203531_at | 4.39 | 1.56 | 2.81 | − | CUL5 | cullin 5 |
| 213743_at | 11.99 | 4.29 | 2.79 | − | CCNT2 | cyclin T2 |
| 217301_x_at | 21.86 | 8.16 | 2.68 | + | RBBP4 | retinoblastoma binding protein 4 |
| 202388_at | 64.82 | 24.87 | 2.61 | − | RGS2 | regulator of G-protein signalling 2, 24 kDa |
| 209903_s_at | 10.39 | 4.17 | 2.49 | − | ATR | ataxia telangiectasia and Rad3 related |
| 205245_at | 8.76 | 3.79 | 2.32 | + | PARD6A | par-6 partitioning defective 6 homolog alpha |
| | | | | | | (C. elegans) |
| 213151_s_at | 2.56 | 1.13 | 2.27 | − | 38967 | septin 7 |
| 212332_at | 63.97 | 29.53 | 2.17 | + | RBL2 | retinoblastoma-like 2 (p130) |
| 205895_s_at | 6.88 | 3.26 | 2.11 | + | NOLC1 | nucleolar and coiled-body phosphoprotein 1 |
| 206967_at | 19.89 | 9.81 | 2.03 | + | CCNT1 | cyclin T1 |

In ER-negative tumors, examples of pathways with genes that had both positive or negative correlation to DMFS include Regulation of cell growth (FIG. 2b), the most significant pathway (Table 2), and Cell adhesion (FIG. 2d). Of the top 20 pathways in ER-negative tumors, none showed a dominant positive association with DMFS, but some did display a dominant negative correlation (FIG. 6 online) including Regulation of G-protein coupled receptor signaling (FIG. 2f), Skeletal development (FIG. 2h), and the pathways ranked among the top 3 in significance (Table 2). Of the top 20 core pathways 4 overlapped between ER-positive and -negative tumors, i.e., Regulation of cell cycle, Protein amino acid phosphorylation, Protein biosynthesis, and Cell cycle (Table 2).

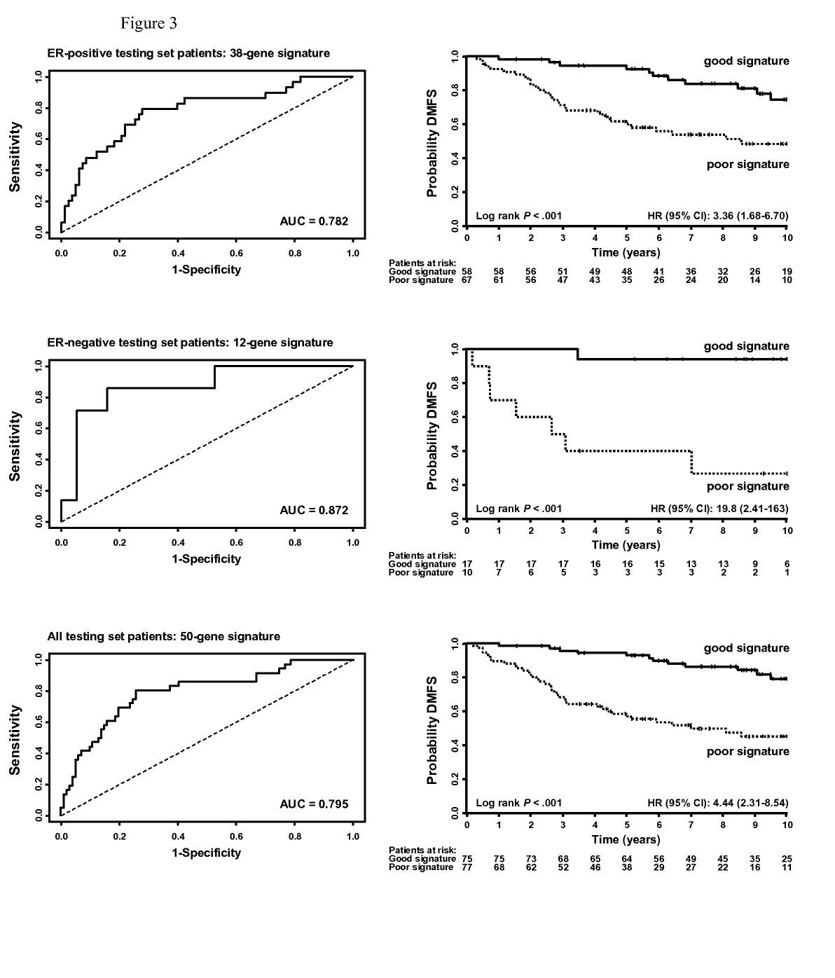

In an attempt to use gene expression profiles in the most significant biological processes to predict distant metastases we used the genes of the top 2 significant pathways in both ER-positive and -negative tumors (Table 7) to construct a gene signature for prediction of distant recurrence. A 50-gene signature was constructed by combining the 38 genes from the top 2 ER-positive pathways and 12 genes for the top 2 ER-negative pathways. The Affymetrix U133A data on a recently published set of breast tumors with follow-up information²¹ was used as an independent test set to validate the signature. The 152-patient validation set consisted of 125 ER-positive tumors and 27 ER-negative tumors. When the 38-gene signature was applied to ER-positive tumors, an ROC analysis gave an AUC of 0.782 (FIG. 3a), and Kaplan-Meier analysis for DMFS showed a clear separation in risk groups

| Probe Set | SD* | z-Score | DMFS† | Gene Symbol | Gene Title |
|---|---|---|---|---|---|
| 208905_at | 3.04 | 4.29 | − | CYCS | cytochrome c, somatic |
| 204817_at | 9.77 | 3.73 | − | ESPL1 | extra spindle poles like 1 |
| 38158_at | 7.23 | 3.41 | − | ESPL1 | extra spindle poles like 1 |
| 204947_at | 16.65 | 3.04 | − | E2F1 | E2F transcription factor 1 |
| 201111_at | 6.18 | 3.04 | − | CSE1L | CSE1 chromosome segregation 1-like |
| 201636_at | 2.34 | 2.97 | − | FXR1 | fragile X mental retardation, autosomal homolog 1 |
| 220048_at | 1.28 | 2.82 | − | EDAR | ectodysplasin A receptor |
| 210766_s_at | 4.54 | 2.75 | − | CSE1L | CSE1 chromosome segregation 1-like |
| 221567_at | 6.81 | 2.66 | − | NOL3 | nucleolar protein 3 (apoptosis repressor with CARD domain) |
| 213829_x_at | 2.54 | 2.65 | − | TNFRSF6B | tumor necrosis factor receptor superfamily, member 6b, decoy |
| 201112_s_at | 2.79 | 2.57 | − | CSE1L | CSE1 chromosome segregation 1-like |
| 212353_at | 10.77 | 2.51 | − | SULF1 | sulfatase 1 |
| 208822_s_at | 1.81 | 2.47 | − | DAP3 | death associated protein 3 |
| 209462_at | 36.92 | 2.37 | − | APLP1 | amyloid beta (A4) precursor-like protein 1 |
| 203005_at | 1.98 | 2.29 | − | LTBR | lymphotoxin beta receptor (TNFR superfamily, member 3) |
| 202731_at | 11.50 | 4.01 | + | PDCD4 | programmed cell death 4 |
| 206150_at | 18.92 | 3.57 | + | TNFRSF7 | tumor necrosis factor receptor superfamily, member 7 |
| 202730_s_at | 8.73 | 3.18 | + | PDCD4 | programmed cell death 4 |
| 209539_at | 9.89 | 3.14 | + | ARHGEF6 | Rac/Cdc42 guanine nucleotide exchange factor (GEF) 6 |
| 212593_s_at | 12.82 | 3.07 | + | PDCD4 | programmed cell death 4 |
| 204933_s_at | 45.18 | 2.96 | + | TNFRSF11B | tumor necrosis factor receptor superfamily, member 11b |
| 209831_x_at | 2.59 | 2.43 | + | DNASF2 | deoxyribonuclease II, lysosomal |
| 203187_at | 3.21 | 2.38 | + | DOCK1 | dedicator of cytokinesis 1 |
| 210164_at | 23.24 | 2.34 | + | GZMB | granzyme B |

(HR=3.36) (FIG. 3b). For the 12-gene signature for ER-negative tumors, an AUC of 0.872 (FIG. 3c) and a HR of 19.8 (FIG. 3d) were obtained. The combined 50-gene signature for ER-positive and ER-negative tumors gave an AUC of 0.795 (FIG. 3e) and a HR of 4.44 (FIG. 3f). Thus a gene signature can now be derived by combining statistical methods and biological knowledge. The present invention provides not only a new way to derive gene signatures for cancer prognosis, but also an insight to the distinct biological processes between subgroups of tumors.

TABLE 7
Genes used for prediction in top pathways
Significant genes in the Apoptosis pathways in ER-positive tumors
Significant genes in the Regulation of cell cycle pathway in ER-positive tumors

| | | | | | |
|---|---|---|---|---|---|
| Probe Set | SD* | z-Score | DMFS† | Gene Symbol | Gene Title |

Significant genes in the Regulation of cell growth pathway in ER-negative tumors

| | | | | | |
|---|---|---|---|---|---|
| 204817_at | 8.90 | 3.73 | − | ESPL1 | extra spindle poles like 1 (S. cerevisiae) |
| 38158_at | 6.60 | 3.41 | − | ESPL1 | extra spindle poles like 1 (S. cerevisiae) |
| 214710_s_at | 7.19 | 3.10 | − | CCNB1 | cyclin B1 |
| 212426_s_at | 2.55 | 3.08 | − | YWHAQ | tyrosine 3-/tryptophan 5-monooxygenase activation protein |
| 204009_s_at | 2.53 | 3.08 | − | KRAS | v-Ki-ras2 Kirsten rat sarcoma viral oncogene homolog |
| 204947_at | 15.18 | 3.04 | − | E2F1 | E2F transcription factor 1 |
| 201947_s_at | 2.30 | 3.04 | − | CCT2 | chaperonin containing TCP1, subunit 2 (beta) |
| 204822_at | 14.49 | 2.91 | − | TTK | TTK protein kinase |
| 209096_at | 2.77 | 2.57 | − | UBE2V2 | ubiquitin-conjugating enzyme E2 variant 2 |
| 204826_at | 4.33 | 2.53 | − | CCNF | cyclin F |
| 212022_s_at | 14.44 | 2.46 | − | MKI67 | antigen identified by monoclonal antibody Ki-67 |
| 202647_s_at | 3.41 | 2.42 | − | NRAS | neuroblastoma RAS viral (v-ras) oncogene homolog |
| 201076_at | 2.43 | 3.09 | + | NHP2L1 | NHP2 non-histone chromosome protein 2-like 1 (S. cerevisiae) |
| 201601_x_at | 8.16 | 3.00 | + | IFITM1 | interferon induced transmembrane protein 1 (9-27) |
| 204015_s_at | 24.75 | 2.90 | + | DUSP4 | dual specificity phosphatase 4 |
| 220407_s_at | 6.36 | 2.68 | + | TGFB2 | transforming growth factor, beta 2 |
| 206404_at | 10.98 | 2.38 | + | FGF9 | fibroblast growth factor 9 (glia-activating factor) |
| 209648_x_at | 5.77 | 4.01 | − | SOC55 | suppressor of cytokine signaling 5 |
| 208127_s_at | 3.71 | 3.75 | − | SOC55 | suppressor of cytokine signaling 5 |
| 209550_at | 5.88 | 3.18 | − | NDN | necdin homolog (mouse) |
| 201162_at | 5.15 | 3.14 | − | IGFBP7 | insulin-like growth factor binding protein 7 |
| 213910_at | 12.99 | 2.87 | − | IGFBP7 | insulin-like growth factor binding protein 7 |
| 212279_at | 4.53 | 2.91 | + | MAC30 | hypothetical protein MAC30 |
| 213337_s_at | 2.53 | 2.88 | + | SOCS1 | suppressor of cytokine signaling 1 |

Significant genes in the Regulation of G-protein coupled receptor signaling pathway
in ER-negative tumors

| | | | | | |
|---|---|---|---|---|---|
| 204337_at | 7.89 | 3.99 | − | RGS4 | regulator of G-protein signalling 4 |
| 209324_s_at | 2.73 | 3.73 | − | RGS16 | regulator of G-protein signalling 16 |
| 220300_at | 3.61 | 2.61 | − | RGS3 | regulator of G-protein signalling 3 |
| 202388_at | 9.45 | 2.61 | − | RGS2 | regulator of G-protein signalling 2, 24 kDa |
| 204396_s_at | 2.47 | 2.34 | − | GRK5 | G protein-coupled receptor kinase 5 |

*SD = Standard deviation
†DMFS = distant metastasis-free survival;
+ = positive correlation with DMFS,
− = negative correlation with DMFS

To compare genes from various prognostic signatures for breast cancer, five published gene signatures were selected^{6,8,21-23}. We first compared the gene sequence identity between each pair of the gene signatures and found very few overlapping genes as expected (Table 8). The gene expression grade index comprising 97 genes, of which most are associated with cell cycle regulation and proliferation²¹, showed the highest number of overlapping genes between the various signatures ranging from 5 with the 16 genes of Genomic Health²² to 10 with Yu's 62 genes²³. The other 4 gene signatures showed only 1 gene overlap in pair-wise comparison, and there was no common gene for all signatures. In spite of the low number of overlapping genes across signatures, which are due to different platforms and bioinformatical analyses used and different groups of patients analyzed, we found that the representation of common pathways in the various signatures may underlie their individual prognostic value⁸. Therefore, we examined the representation of the top 20 core pathways (Table 2) in the 5 signatures, the genes in the signatures were mapped to GOBP. Except the Genomic Health 16-gene signature mapped to 10 distinct core pathways, each of the other 4 signatures with 62 genes or more mapped to 19 distinct core prognostic pathways (Table 3). Of these 19 pathways, 8 were identical for all 4 signatures, i.e., Mitosis, Apoptosis, Regulation of cell cycle, DNA repair, Cell cycle, Protein amino acid phosphorylation, Intracellular signaling cascade, and Cell adhesion. The other 11 pathways were either present in 1, 2, or 3, of the signatures, but not in all (Table 3). In a recent study, comparing the prognostic performance of different gene signatures, agreement in outcome predictions were found as well²⁴. However, in contrast to our present approach, the underlying pathways were not investigated, and merely the performance of various gene signatures on a single patient cohort, heterogeneous with respect to nodal status and adjuvant systemic therapy²⁵, was compared²⁴. It is important to note, however, that although similar pathways are represented in various signatures, it does not necessarily mean the individual genes in a pathway contribute equally and into the same direction. Genes in a specific pathway may be positively or negatively associated with tumor aggressiveness, and have very different contributions and significance levels (FIGS. 5 and 6, and Tables 5 and 6).

TABLE 8
Number of common genes between different gene signatures for breast cancer prognosis

| | | | Genomic | |
|---|---|---|---|---|
| | Wang's 76 | van't Veer's 70 | Health 16 | |
| | genes | genes | genes | Yu's 62 genes |
| Wang's 76 | | CCNE2 | No genes | No genes |
| genes* | | | | |
| van 't Veer's | CNNE2 | | SCUBE2 | AA962149 |
| 70 genes† | | | | |
| Genomic | No genes | SCUBE2 | | BIRC5 |
| Health 16 | | | | |
| genes‡ | | | | |
| Yu's 62 genes* | No genes | AA962149 | BIRC5 | |
| Sotiriou's 97 | PLK1, FEN1, | MELK, | MYBL2, | URCC6, FOXM1, |
| genes* | CCNE2, | CENPA, | BIRC5, STK6, | DLG7, |
| | GTSE1, | CCNE2, | MKI67, | DKFZp686L20222, |
| | KPNA2, | GMPS, DC13, | CCNB1 | DC13, FLJ32241, |
| | MLF1IP, | PRC1, | | HSP1CDC21, CDC2, |
| | POLQ | NUSAP1, | | KIF11, EXO1 |
| | | KNTC2 | | |

*Affymetrix HG-U133A Genechip
†Agilent Hu25K microarray
‡No genome-wide assessment; RT-PCR

TABLE 3
Mapping various gene signatures to core pathways

| | | Published gene signatures^{a} | | | | |
|---|---|---|---|---|---|---|
| Pathways | GO_ID | Wang | Van 't Veer | Paik | Yu | Sotiriou |
| ER-positive tumors | | | | | | |
| Apoptosis | 6915 | X | X | X | X | X |
| Regulation of cell cycle | 74 | X | X | X | X | X |
| Protein amino acid phosphorylation | 6468 | X | X | X | X | X |
| Cytokinesis | 910 | X | X | X | | X |
| Cell motility | 6928 | | | | X | X |
| Cell cycle | 7049 | X | X | X | X | X |
| Cell surface receptor-linked signal transduction | 7166 | | | X | | |
| Mitosis | 7067 | X | X | X | X | X |
| Intracellular protein transport | 6886 | X | X | | | X |
| Mitotic chromosome segregation | 70 | X | X | | | X |
| Ubiquitin-dependent protein catabolism | 6511 | | X | | X | X |
| DNA repair | 6281 | X | X | | X | X |
| Induction of apoptosis | 6917 | X | | | | |
| Immune response | 6955 | X | | | X | X |
| Protein biosynthesis | 6412 | | | X | X | X |
| DNA replication | 6260 | X | X | | X | X |
| Oncogenesis | 7048 | | | X | X | X |
| Metabolism | 8152 | X | X | | | |
| Cellular defense response | 6968 | X | | | X | X |
| Chemotaxis | 6935 | | | | X | X |
| ER-negative tumors | | | | | | |
| Regulation of cell growth | 1558 | | X | | | |
| Regulation of G-coupled receptor signaling | 8277 | | | | | |
| Skeletal development | 1501 | X | X | | | |
| Protein amino acid phosphorylation | 6468 | X | X | X | X | X |
| Cell adhesion | 7155 | X | X | | X | X |
| Carbohydrate metabolism | 5975 | X | X | | | |
| Nuclear mRNA splicing, via spliceosome | 398 | | | | | |
| Signal transduction | 7165 | X | X | X | X | |
| Cation transport | 6812 | | | | | |
| Calciumion transport | 6816 | | | | | |
| Protein modification | 6464 | | | | | |
| Intracellular signaling cascade | 7242 | X | X | | X | X |
| mRNA processing | 6397 | | | | | |
| RNA splicing | 8380 | | | | | |
| Endocytosis | 6897 | | | | | |
| Regulation of transcription from PolII promoter | 6357 | | | | X | |
| Regulation of cell cycle | 74 | X | X | X | | |
| Protein complex assembly | 6461 | | X | | X | |
| Protein biosynthesis | 6412 | | | X | | X |
| Cell cycle | 7049 | X | X | X | X | X |

^{a}Published gene signatures that were studied include the 76-gene signature by Wang et al⁸, the 70-gene signature by van 't Veer et al⁶, the 16-gene signature by Paik et al²², the 62-gene signature by Yu et al²³, and the 97-gene signature by Sotiriou et al²¹. Individual genes in each signature were mapped to the top 20 core pathways for ER-positive and ER-negative tumors.

In conclusion, we have shown that gene signatures can be derived by combining statistical methods and biological knowledge. Our study for the first time applied a method that systematically evaluated the biological pathways related to patient outcomes of breast cancer and have provided biological evidence that various published prognostic gene signatures providing similar outcome predictions are based on the representation of common biological processes. Identification of the key biological processes, rather than the assessment of signatures based on individual genes, provides targets for future drug development.

The following examples are provided to illustrate but not limit the claimed invention. All references cited herein are hereby incorporated herein by reference.

### EXAMPLE 1

### Methods

Patient population. The study was approved by the Medical Ethics Committee of the Erasmus MC Rotterdam, The Netherlands (MEC 02.953), and was performed in accordance to the Code of Conduct of the Federation of Medical Scientific Societies in the Netherlands (www.fmwv.nl). A cohort of 344 breast tumor samples from a tumor bank at the Erasmus Medical Center (Rotterdam, Netherlands) were used in this study. All these samples were from patients with lymph node-negative breast cancer who had not received any adjuvant systemic therapy, and had more than 70% tumor content. Among them, 286 samples had been used to derive a 76-gene signature to predict distant metastasis⁸. An additional 58 ER-negative cases were included to increase the numbers in this subgroup in the analyses performed. In this study, ER status for a patient was determined based on the expression level of the ER gene on the chip. A patient is considered ER-positive if its ER expression level is higher than 1000 after scaling the average of intensity on a chip to 600. Otherwise, the patient is ER-negative²⁶. As a result, there were 221 ER-positive and 123 ER-negative patients in the 344-patient population. The mean age of the patients was 53 years (median 52, range 26-83 years), 175 (51%) were premenopausal and 169 (49%) postmenopausal. T1 tumors (≦2 cm) were present 168 patients (49%), T2 tumors (>2-5 cm) in 163 patients (47%), T3/4 tumors (>5 cm) in 12 patients (3%), and 1 patient with unknown tumor stage. Pathological examination was carried out by regional pathologists as described previously²⁷ and the histological grade was coded as poor in 184 patients (54%), moderate in 45 patients (13%, good in 7 patients (2%), and unknown for 108 patients (31%). During follow-up 103 patients showed a relapse within 5 years and were counted as failures in the analysis for DMFS. Eighty two patients died after a previous relapse. The median follow-up time of patients still alive was 101 months (range 61-171 months).

RNA isolation and hybridization. Total RNA was extracted from 20-40 cryostat sections of 30 um thickness with RNAzol B (Campro Scientific, Veenendaal, Netherlands). After being biotinylated, targets were hybridized to Affymetrix HG-U133A chips as described⁸. Gene expression signals were calculated using Affymetrix GeneChip analysis software MAS 5.0. Chips with an average intensity less than 40 or a background higher than 100 were removed. Global scaling was performed to bring the average signal intensity of a chip to a target of 600 before data analysis.

For the validation dataset²¹, quantile normalization was performed and ANOVA was used to eliminate batch effects from different sample preparation methods, RNA extraction methods, different hybridization protocols and scanners.

Multiple gene signatures. Since gene expression patterns of ER-positive breast tumors are quite different from that of ER-negative breast tumors⁸, data analysis to derive gene signatures and subsequent pathway analysis were conducted separately. For either ER-positive or ER-negative patients, 80 samples were randomly selected as a training set. For the training set, univariant Cox proportional-hazards regression was performed to identify genes whose expression patterns were most correlated to patients' distant metastasis-free survival (DMFS) time. Our previous analysis suggested that 80 patients represent a minimum size of the training set for producing a prognostic gene signature of stable performance⁸. The top 100 genes were used as a signature to predict tumor recurrence for the remaining independent patients as a test set. A receiver operating characteristic (ROC) analysis with distant metastasis within 5 years as a defining point was conducted. The area under curve (AUC) was used as a measurement of the performance of a signature in the test set. The above procedure was repeated 500 times (FIG. 4). Thus, 500 signatures of 100 genes each were obtained. The frequency of the selected genes in the 500 signatures was calculated and the genes were ranked based on the frequency.

As a control, the patient clinical information for the ER-positive patients or ER-negative patients was permutated randomly and reassigned to the chip data. As described above, 80 chips were then randomly selected as a training set and the top 100 genes were selected using the Cox modeling based on the permutated clinical information. The top 100 genes were then used as a signature to predict relapse in the remaining patients. The clinical information was permutated 10 times. For each permutation of the clinical information, 50 various training sets of 80 patients were created. For each training set, the top 100 genes were obtained as a control gene list based on the Cox modeling. Thus, a total of 500 control signatures were obtained. The predictive performance of the 100 genes was examined in the remaining patients. An ROC analysis was conducted and AUC was calculated in the test set.

Mapping to GOBP. To identify over-representation of biological pathways in the signatures, genes on Affymetrix HG-U133A chip were mapped to the categories of GOBP based on the annotation table downloaded from www.affymetrix.com. Categories that contain at least 10 probe sets from HG-U133A chip were retained for subsequent pathway analysis. The 100 genes of each signature were mapped to GOBP. Hypergeometric distribution probabilities for GOBP categories were calculated for each signature. A pathway that has a hypergeometric distribution probability <0.05 and was hit by two or more genes from the 100 genes was considered as an over-represented pathway in a signature. The total number of a pathway appeared in the 500 signatures was considered as the frequency of over-representation.

Global Test program. To evaluate the relationship between a pathway and the clinical outcome, each of the top 20 over-represented pathways that have the highest frequencies in the 500 signatures were subjected to Global Test program^{1,2}. The Global Test examines the association of a group of genes as a whole to a specific clinical parameter such as DMFS. The contribution of individual genes in the top over-represented pathways to the association was also evaluated and significant contributors were selected for subsequent analyses.

To explore the possibility of using the genes in a specific pathway as a signature to predict distant metastasis, the top two pathways for ER-positive or ER-negative tumors that were in the top 20 list based on frequency of over-representation and had the smallest P values from Global Test program were chosen to build a gene signature. First, genes in the pathway were selected if their z-score was greater than 1.95 from the Global Test program. A z-score greater than 1.95 indicates that the association of the gene expression with DMFS time is significant (P<0.05)^{1,2}. The relapse score was the difference of weighted expression signals for negatively correlated genes and ones for positively correlated genes. To determine the optimal number of genes in a signature, ROC analysis was performed using signatures of various numbers of genes in the training set. The performance of the selected gene signature was evaluated by Kaplan-Meier survival analysis in an independent patient group²¹.

Comparing multiple gene signatures. To compare the genes from various prognostic signatures for breast cancer, five gene signatures were selected^{6,8,22-23}. Identity of the genes between the signatures was determined by BLAST program. To examine the representation of the top 20 pathways in the signatures, genes in each of the signatures were mapped to GOBP.

Data Availability. The microarray data analyzed in this paper have been submitted to the NCBI/Genbank GEO database. The microarray and clinical data used for the independent validation testing set analysis were obtained from the Gene Expression Omnibus database (http://www.ncbi.nlm.hih.gov.geo) with accession code GSE2990.

Statistical Methods. Statistical analyses were conducted using the R system, version 2.2.1 (http://www.r-project.org). Cox proportional-hazard regression modeling analysis was performed to identify genes with a high correlation to DMFS in each training set. The survival package included in the R system was used for survival analysis. The hazard ratio (HR) and 95% confidence intervals (CI) were estimated using the stratified Cox regression analysis. Hypergeometric distribution probability analysis was performed to identify over-represented pathways in each of the 500 signatures. Global Test, version 3.1.1, was used to evaluate the top over-represented pathways related to DMFS and provided a way to visualize contributions of individual genes in a pathway.

Although the foregoing invention has been described in some detail by way of illustration and example for purposes of clarity of understanding, the descriptions and examples should not be construed as limiting the scope of the invention.

### REFERENCES

(1) Goeman, J. J., van de Geer, S. A., de Kort, F. & van Houwelingen, H. C. A global test for groups of genes: testing association with a clinical outcome. Bioinformatics 20, 93-99 (2004).
(2) Goeman, J. J., Oosting, J., Cleton-Jansen, A. M., Anning a, J. K. & van Houwelingen, H. C. Testing association of a pathway with survival using gene expression data. Bioinformatics 21, 1950-1957 (2005).
(3) Perou, C. M. et al. Molecular portraits of human breast tumours. Nature 406, 747-752 (2000).
(4) Sorlie, T. et al. Gene expression patterns of breast carcinomas distinguish tumor subclasses with clinical implications. Proc. Natl. Acad. Sci. U.S.A. 98, 10869-10874 (2001).
(5) Sorlie, T. et al. Repeated observation of breast tumor subtypes in independent gene expression data sets. Proc. Natl. Acad. Sci. U.S.A. 100, 8418-8423 (2003).
(6) van 't Veer, L. J. et al. Gene expression profiling predicts clinical outcome of breast cancer. Nature 415, 530-536 (2002).
(7) Sotiriou, C. et al. Breast cancer classification and prognosis based on gene expression profiles from a population-based study. Proc. Natl. Acad. Sci. U.S.A. 100, 10393-10398 (2003).
(8) Wang, Y. et al. Gene-expression profiles to predict distant metastasis of lymph-node-negative primary breast cancer. Lancet 365, 671-679 (2005).
(9) Jansen, M. P. H. M. et al. Molecular classification of tamoxifen-resistant breast carcinomas by gene expression profiling. J. Clin. Oncol. 23, 732-740 (2005).
(10) Brenton, J. D., Carey, L. A., Ahmed, A. A. & Caldas, C. Molecular classification and molecular forecasting of breast cancer: ready for clinical application? J. Clin. Oncol. 23, 7350-7360 (2005).
(11) Smid, M. et al. Genes associated with breast cancer metastatic to bone. J. Clin. Oncol. 24, 2261-2267 (2006).
(12) Michiels, S., Koscielny, S. & Hill, C. Prediction of cancer outcome with microarrays: a multiple random validation strategy. Lancet 365, 488-492 (2005).
(13) Tinker, A. V., Boussioutas, A. & Bowtell, D. D. L. The challenges of gene expression microarrays for the study of human cancer. Cancer Cell 9, 333-939 (2006).
(14) Vogelstein, B. & Kinzler, K. W. Cancer genes and the pathways they control. Nature Med. 8, 789-798 (2004).
(15) Segal, E., Friedman, N., Kaminski, N., Regev, A. & Koller, D. From signatures to models: understanding cancer using microarrays. Nature Genet. Suppl. 37, S38-45 (2005).
(16) Tian, L. et al. Discovering statistically significant pathways in expression profiling studies. Proc. Natl. Acad. Sci. U.S.A. 102, 13544-13549 (2005).
(17) Subramanian, A. et al. Gene set enrichment analysis: a knowledge-based approach for interpreting genome-wide expression profiles. Proc. Natl. Acad. Sci. U.S.A. 102, 15545-15550 (2005).
(18) Bild, A. H. et al. Oncogenic pathway signatures in human cancers as a guide to targeted therapies. Nature 439, 353-357 (2006).
(19) Adler, A. S. et al. Genetic regulators of large-scale transcriptional signatures in cancer. Nature Genet. 4, 421-430 (2006).
(20) Gruvberger, S. et al. Estrogen receptor status in breast cancer is associated with remarkable distinct gene expression patterns. Cancer Res. 61, 5979-5984 (2001).
(21) Sotiriou, C. et al. Gene expression profiling in breast cancer: understanding the molecular basis for histologic grade to improve prognosis. J. Natl. Cancer Inst. 98, 262-272 (2006).
(22) Paik, S. et al. A multigene assay to predict recurrence of tamoxifen-treated, node-negative breast cancer. N. Eng. J. Med. 351, 2817-2825 (2004).
(23) Yu, K. et al. A molecular signature of the Nottingham prognostic index in breast cancer. Cancer Res. 64, 2962-2968 (2004).
(24) Fan, C. et al. Concordance among gene-expression-based predictors for breast cancer. N. Engl. J. Med. 355, 560-569 (2006).
(25) van de Vijver, M. J. et al. A gene-expression signature as a predictor of survival in breast cancer. N. Engl. J. Med. 347, 1999-2009 (2002).
(26) Foekens, J. A. et al. Multicenter validation of a gene expression-based prognostic signature in lymph node-negative primary breast cancer. J. Clin. Oncol. 24, 1665-1671 (2006).
(27) Foekens, J. A. et al. Prognostic value of receptors for insulin-like growth factor 1, somatostatin, and epidermal growth factor in human breast cancer. Cancer Res. 49, 7002-7009 (1989).

Gene descriptions and SEQ ID NOS:

| SEQ | | | | |
|---|---|---|---|---|
| ID | | | | |
| NO: | Accession | Name | Description | PSID |
| 1 | | KIAA0241 | KIAA0241 protein | |
| 2 | | CD44 | CD44 antigen (homing function and Indian blood | |
| | | | group system) | |
| 3 | | ABCC5 | ATP-binding cassette, sub-family C (CFTR/MRP), | |
| | | | member 5 | |
| 4 | | STK6 | serine/threonine kinase 6 | |
| 5 | | CYCS | cytochrome c, somatic | |
| 6 | | KIA0406 | KIAA0406 gene product | |
| 7 | | UCKL1 | uridine-cytidine kinase 1-like 1 | |
| 8 | | ZCCHC8 | zinc finger, CCHC domain containing 8 | |
| 9 | | RACGAP1 | Rac GTPase activating protein 1 | |
| 10 | | STAU | staufen, RNA binding protein (Drosophila) | |
| 11 | | LACTB2 | lactamase, beta 2 | |
| 12 | | EEF1A2 | eukaryotic translation elongation factor 1 alpha 2 | |
| 13 | | RAE1 | RAE1 RNA export 1 homolog (S. pombe) | |
| 14 | | TUFT1 | tuftelin 1 | |
| 15 | | ZFP36L2 | zinc finger protein 36, C3H type-like 2 | |
| 16 | | ORC6L | origin recognition complex, subunit 6 homolog- | |
| | | | like (yeast) | |
| 17 | | ZNF623 | zinc finger protein 623 | |
| 18 | | ESPL1 | extra spindle poles like 1 | |
| 19 | | TCEB1 | transcription elongation factor B (SIII), | |
| | | | polypeptide 1 | |
| 20 | | RPS6KB1 | ribosomal protein S6 kinase, 70 kDa, polypeptide 1 | |
| 21 | | ZFPM2 | zinc finger protein, multitype 2 | |
| 22 | | RPL26L1 | ribosomal protein L26-like 1 | |
| 23 | | FLJ14346 | hypothetical protein FLJ14346 | |
| 24 | | MAPKAPK2 | mitogen-activated protein kinase-activated | |
| | | | protein kinase 2 | |
| 25 | | COL2A1 | collagen, type II, alpha 1 | |
| 26 | | MBNL2 | muscleblind-like 2 (Drosophila) | |
| 27 | | GPR124 | G protein-coupled receptor 124 | |
| 28 | | SFRS11 | splicing factor, arginine/serine-rich 11 | |
| 29 | | HNRPA1 | heterogeneous nuclear ribonucleoprotein A1 | |
| 30 | | CDC42BPA | CDC42 binding protein kinase alpha (DMPK-like) | |
| 31 | | RGS4 | regulator of G-protein signalling 4 | |
| 32 | | TRPC1 | transient receptor potential cation channel, | |
| | | | subfamily C, member 1 | |
| 33 | | TCF8 | transcription factor 8 (represses interleukin 2 | |
| | | | expression) | |
| 34 | | C6orf210 | chromosome 6 open reading frame 210 | |
| 35 | | DNM3 | dynamin 3 | |
| 36 | | Cep63 | centrosome protein Cep63 | |
| 37 | | TNFSF13 | tumor necrosis factor (ligand) superfamily, | |
| | | | member 13 | |
| 38 | | DACT1 | dapper, antagonist of beta-catenin, homolog 1 | |
| | | | (Xenopus laevis) | |
| 39 | | RECK | reversion-inducing-cysteine-rich protein with | |
| | | | kazal motifs | |
| 40 | | CYCS | cytochrome c, somatic | 208905_at |
| 41 | | PDCD4 | programmed cell death 4 | 202731_at |
| 42 | | ESPL1 | extra spindle poles like 1 | 204817_at |
| 43 | | TNFRSF7 | tumor necrosis factor receptor superfamily, | 206150_at |
| | | | member 7 | |
| 44 | | ESPL1 | extra spindle poles like 1 | 38158_at |
| 45 | | PDCD4 | programmed cell death 4 | 202730_s_at |
| 46 | | ARHGEF6 | Rac/Cdc42 guanine nucleotide exchange factor | 209539_at |
| | | | (GEF) 6 | |
| 47 | | PDCD4 | programmed cell death 4 | 212593_s_at |
| 48 | | E2F1 | E2F transcription factor 1 | 204947_at |
| 49 | | CSE1L | CSE1 chromosome segregation 1-like | 201111_at |
| 50 | | FXR1 | fragile X mental retardation, autosomal homolog 1 | 201636_at |
| 51 | | TNFRSF11B | tumor necrosis factor receptor superfamily, | 204933_s_at |
| | | | member 11b | |
| 52 | | EDAR | ectodysplasin A receptor | 220048_at |
| 53 | | CSE1L | CSE1 chromosome segregation 1-like (yeast) | 210766_s_at |
| 54 | | NOL3 | nucleolar protein 3 (apoptosis repressor with | 221567_at |
| | | | CARD domain) | |
| 55 | | TNFRSF6B | tumor necrosis factor receptor superfamily, | 213829_x_at |
| | | | member 6b, decoy | |
| 56 | | CSE1L | CSE1 chromosome segregation 1-like | 201112_s_at |
| 57 | | SULF1 | sulfatase 1 | 212353_at |
| 58 | | DAP3 | death associated protein 3 | 208822_s_at |
| 59 | | DNASE2 | deoxyribonuclease II, lysosomal | 209831_x_at |
| 60 | | DOCK1 | dedicator of cytokinesis 1 | 203187_at |
| 61 | | APLP1 | amyloid beta (A4) precursor-like protein 1 | 209462_at |
| 62 | | GZMB | granzyme B | 210164_at |
| 63 | | LTBR | lymphotoxin beta receptor | 203005_at |
| 64 | | NFKB1 | nuclear factor of kappa light polypeptide gene | 209239_at |
| | | | enhancer in B-cells 1 (p105) | |
| 65 | | FADD | Fas (TNFRSF6)-associated via death domain | 202535_at |
| 66 | | PHLDA2 | pleckstrin homology-like domain, family A, | 209803_s_at |
| | | | member 2 | |
| 67 | | ELMO1 | engulfment and cell motility 1 (ced-12 homolog, C. elegans) | 204513_s_at |
| 68 | | BIRC3 | baculoviral IAP repeat-containing 3 | 210538_s_at |
| 69 | | DDX41 | DEAD (Asp-Glu-Ala-Asp) box polypeptide 41 | 217840_at |
| 70 | | IL17 | interleukin 17 (cytotoxic T-lymphocyte-associated | 208402_at |
| | | | serine esterase 8) | |
| 71 | | DNASE2 | deoxyribonuclease II, lysosomal | 214992_s_at |
| 72 | | CXCR4 | chemokine (C—X—C motif) receptor 4 | 209201_x_at |
| 73 | | E2F1 | E2F transcription factor 1 | 2028_s_at |
| 74 | | TXNL1 | thioredoxin-like 1 | 201588_at |
| 75 | | MAP3K5 | mitogen-activated protein kinase kinase kinase 5 | 203836_s_at |
| 76 | | FAS | Fas (TNF receptor superfamily, member 6) | 215719_x_at |
| 77 | | CCNB1 | cyclin B1 | 214710_s_at |
| 78 | | NHP2L1 | NHP2 non-histone chromosome protein 2-like 1 | 201076_at |
| 79 | | YWHAQ | tyrosine 3-monooxygenase/tryptophan 5- | 212426_s_at |
| | | | monooxygenase activation protein | |
| 80 | | KRAS | v-Ki-ras2 Kirsten rat sarcoma viral oncogene | 204009_s_at |
| | | | homolog | |
| 81 | | CCT2 | chaperonin containing TCP1, subunit 2 (beta) | 201947_s_at |
| 82 | | IFITM1 | interferon induced transmembrane protein 1 (9-27) | 201601_x_at |
| 83 | | TTK | TTK protein kinase | 204822_at |
| 84 | | DUSP4 | dual specificity phosphatase 4 | 204015_s_at |
| 85 | | TGFB2 | transforming growth factor, beta 2 | 220407_s_at |
| 86 | | UBE2V2 | ubiquitin-conjugating enzyme E2 variant 2 | 209096_at |
| 87 | | CCNF | cyclin F | 204826_at |
| 88 | | MKI67 | antigen identified by monoclonal antibody Ki-67 | 212022_s_at |
| 89 | | NRAS | neuroblastoma RAS viral (v-ras) oncogene | 202647_s_at |
| | | | homolog | |
| 90 | | FGF9 | fibroblast growth factor 9 (glia-activating factor) | 206404_at |
| 91 | | CCNB2 | cyclin B2 | 202705_at |
| 92 | | CDC20 | CDC20 cell division cycle 20 homolog (S. cerevisiae) | 202870_s_at |
| 93 | | JAK2 | Janus kinase 2 (a protein tyrosine kinase) | 205842_s_at |
| 94 | | IFITM1 | interferon induced transmembrane protein 1 (9-27) | 214022_s_at |
| 95 | | NFYC | nuclear transcription factor Y, gamma | 211251_x_at |
| 96 | | DUSP4 | dual specificity phosphatase 4 | 204014_at |
| 97 | | RBBP6 | retinoblastoma binding protein 6 | 212781_at |
| 98 | | STK6 | serine/threonine kinase 6 | 208079_s_at |
| 99 | | STK6 | serine/threonine kinase 6 | 204092_s_at |
| 100 | | NEK2 | NIMA (never in mitosis gene a)-related kinase 2 | 204641_at |
| 101 | | LYN | v-yes-1 Yamaguchi sarcoma viral related | 210754_s_at |
| | | | oncogene homolog | |
| 102 | | RPS6KC1 | ribosomal protein S6 kinase, 52 kDa, polypeptide 1 | 218909_at |
| 103 | | GMFB | glia maturation factor, beta | 202543_s_at |
| 104 | | MELK | maternal embryonic leucine zipper kinase | 204825_at |
| 105 | | CDC2 | Cell division cycle 2, G1 to S and G2 to M | 203213_at |
| 106 | | RPS6KB1 | ribosomal protein S6 kinase, 70 kDa, polypeptide 1 | 204171_at |
| 107 | | PRKCH | protein kinase C, eta | 218764_at |
| 108 | | CCL2 | chemokine (C-C motif) ligand 2 | 216598_s_at |
| 109 | | BUB1B | BUB1 budding uninhibited by benzimidazoles 1 | 203755_at |
| | | | homolog beta (yeast) | |
| 110 | | TGFBR2 | transforming growth factor, beta receptor II | 208944_at |
| | | | (70/80 kDa) | |
| 111 | | SGK3 | serum/glucocorticoid regulated kinase family, | 220038_at |
| | | | member 3 | |
| 112 | | BUB1 | BUB1 budding uninhibited by benzimidazoles 1 | 209642_at |
| | | | homolog (yeast) | |
| 113 | | ATP6AP1 | ATPase, H+ transporting, lysosomal accessory | 207957_s_at |
| | | | protein 1 | |
| 114 | | HCK | hemopoietic cell kinase | 208018_s_at |
| 115 | | FYN | FYN oncogene related to SRC, FGR, YES | 212486_s_at |
| 116 | | FYN | FYN oncogene related to SRC, FGR, YES | 216033_s_at |
| 117 | | LATS1 | LATS, large tumor suppressor, homolog 1 | 219813_at |
| | | | (Drosophila) | |
| 118 | | NUAK2 | NUAK family, SNF1-like kinase, 2 | 220987_s_at |
| 119 | | NEK7 | NIMA (never in mitosis gene a)-related kinase 7 | 212530_at |
| 120 | | PRKD2 | protein kinase D2 | 209282_at |
| 121 | | SRPK1 | SFRS protein kinase 1 | 202200_s_at |
| 122 | | PRC1 | protein regulator of cytokinesis 1 | 218009_s_at |
| 123 | | CENPE | centromere protein E, 312 kDa | 205046_at |
| 124 | | SMC1L1 | SMC1 structural maintenance of chromosomes 1- | 201589_at |
| | | | like 1 | |
| 125 | | PAFAH1B1 | platelet-activating factor acetylhydrolase, isoform | 200815_s_at |
| | | | lb, alpha subunit 45 kDa | |
| 126 | | PPP1CC | protein phosphatase 1, catalytic subunit, gamma | 200726_at |
| | | | isoform | |
| 127 | | CKS1B | CDC28 protein kinase regulatory subunit 1B | 201897_s_at |
| 128 | | CKS2 | CDC28 protein kinase regulatory subunit 2 | 204170_s_at |
| 129 | | CCNT2 | cyclin T2 | 213743_at |
| 130 | | HMMR | hyaluronan-mediated motility receptor (RHAMM) | 207165_at |
| 131 | | CCR6 | chemokine (C-C motif) receptor 6 | 206983_at |
| 132 | | FN1 | fibronectin 1 | 211719_x_at |
| 133 | | IGF1 | insulin-like growth factor 1 | 211577_s_at |
| 134 | | FN1 | fibronectin 1 | 210495_x_at |
| 135 | | STAT3 | signal transducer and activator of transcription 3 | 208991_at |
| 136 | | TSPAN3 | tetraspanin 3 | 200973_s_at |
| 137 | | FN1 | fibronectin 1 | 216442_x_at |
| 138 | | IGF1 | insulin-like growth factor 1 (somatomedin C) | 209540_at |
| 139 | | CORO1A | coronin, actin binding protein, 1A | 209083_at |
| 140 | | IL8RB | interleukin 8 receptor, beta | 207008_at |
| 141 | | STAT3 | signal transducer and activator of transcription 3 | 208992_s_at |
| 142 | | ACTR3 | ARP3 actin-related protein 3 homolog (yeast) | 213101_s_at |
| 143 | | ARPC2 | actin related protein 2/3 complex, subunit 2, | 208679_s_at |
| | | | 34 kDa | |
| 144 | | SMC4L1 | SMC4 structural maintenance of chromosomes 4- | 201664_at |
| | | | like 1 | |
| 145 | | SMC4L1 | SMC4 structural maintenance of chromosomes 4- | 215623_x_at |
| | | | like 1 | |
| 146 | | HCAP-G | chromosome condensation protein G | 218663_at |
| 147 | | MAD2L1 | MAD2 mitotic arrest deficient-like 1 | 203362_s_at |
| 148 | | JAG2 | jagged 2 | 32137_at |
| 149 | | STRN3 | striatin, calmodulin binding protein 3 | 204496_at |
| 150 | | HCAP-G | chromosome condensation protein G | 218662_s_at |
| 151 | | SMC4L1 | SMC4 structural maintenance of chromosomes 4- | 201663_s_at |
| | | | like 1 | |
| 152 | | RCC1 | regulator of chromosome condensation 1 | 206499_s_at |
| 153 | | CUL4B | cullin 4B | 202214_s_at |
| 154 | | IL27RA | interleukin 27 receptor, alpha | 205926_at |
| 155 | | PTPRC | protein tyrosine phosphatase, receptor type, C | 212587_s_at |
| 156 | | IL6ST | interleukin 6 signal transducer (gp130, oncostatin | 211000_s_at |
| | | | M receptor) | |
| 157 | | KLRB1 | killer cell lectin-like receptor subfamily B, member 1 | 214470_at |
| 158 | | IL27RA | interleukin 27 receptor, alpha | 222062_at |
| 159 | | CENPF | centromere protein F, 350/400ka (mitosin) | 209172_s_at |
| 564 | | KIF2C | kinesin family member 2C | 209408_at |
| 160 | | ERP29 | endoplasmic reticulum protein 29 | 201216_at |
| 161 | | AP2A2 | adaptor-related protein complex 2, alpha 2 subunit | 211779_x_at |
| 162 | | AP2A2 | adaptor-related protein complex 2, alpha 2 subunit | 212159_x_at |
| 163 | | KPNA2 | karyopherin alpha 2 | 201088_at |
| 164 | | RABIF | RAB interacting factor | 204478_s_at |
| 165 | | ARF6 | ADP-ribosylation factor 6 | 203311_s_at |
| 166 | | COPA | coatomer protein complex, subunit alpha | 214337_at |
| 167 | | RAB3A | RAB3A, member RAS oncogene family | 204974_at |
| 168 | | APPBP2 | amyloid beta precursor protein (cytoplasmic tail) | 202630_at |
| | | | binding protein 2 | |
| 169 | | RAB8A | RAB8A, member RAS oncogene family | 208819_at |
| 170 | | VPS45A | vacuolar protein sorting 45A | 209268_at |
| 171 | | VDP | vesicle docking protein p115 | 201831_s_at |
| 172 | | RAB22A | RAB22A, member RAS oncogene family | 218360_at |
| 173 | | TMED1 | transmembrane emp24 protein transport domain | 203679_at |
| | | | containing 1 | |
| 174 | | KIF20A | kinesin family member 20A | 218755_at |
| 175 | | STX3A | syntaxin 3A | 209238_at |
| 176 | | KDELR3 | KDEL (Lys-Asp-Glu-Leu) endoplasmic reticulum | 204017_at |
| | | | protein retention receptor 3 | |
| 177 | | NSF | N-ethylmaleimide-sensitive factor | 202395_at |
| 178 | | RAB33B | RAB33B, member RAS oncogene family | 221014_s_at |
| 179 | | SNX4 | sorting nexin 4 | 212652_s_at |
| 180 | | KPNA6 | Karyopherin alpha 6 (importin alpha 7) | 212103_at |
| 181 | | RABIF | RAB interacting factor | 204477_at |
| 182 | | ARF4 | ADP-ribosylation factor 4 | 201097_s_at |
| 183 | | TNPO1 | Transportin 1 | 212635_at |
| 184 | | STAM | signal transducing adaptor molecule (SH3 domain | 203544_s_at |
| | | | and ITAM motif) 1 | |
| 185 | | KPNA2 | karyopherin alpha 2 (RAG cohort 1, importin alpha | 211762_s_at |
| | | | 1) | |
| 186 | | CLTC | clathrin, heavy polypeptide (Hc) | 200614_at |
| 187 | | RAB2 | RAB2, member RAS oncogene family | 208732_at |
| 188 | | KDELR2 | KDEL (Lys-Asp-Glu-Leu) endoplasmic reticulum | 200699_at |
| | | | protein retention receptor 2 | |
| 189 | | FBXO7 | F-box protein 7 | 201178_at |
| 190 | | PSMB4 | proteasome (prosome, macropain) subunit, beta | 202244_at |
| | | | type, 4 | |
| 191 | | USP32 | ubiquitin specific peptidase 32 | 211702_s_at |
| 192 | | FBXW4 | F-box and WD-40 domain protein 4 | 221519_at |
| 193 | | SIAH1 | seven in absentia homolog 1 (Drosophila) | 202981_x_at |
| 194 | | PSMB8 | proteasome (prosome, macropain) subunit, beta | 209040_s_at |
| | | | type, 8 | |
| 195 | | PSMA6 | proteasome (prosome, macropain) subunit, alpha | 208805_at |
| | | | type, 6 | |
| 196 | | PSMB4 | proteasome (prosome, macropain) subunit, beta | 202243_s_at |
| | | | type, 4 | |
| 197 | | UBE2I | Ubiquitin-conjugating enzyme E2I | 208760_at |
| 198 | | PSMA2 | proteasome (prosome, macropain) subunit, alpha | 201317_s_at |
| | | | type, 2 | |
| 199 | | POLQ | polymerase (DNA directed), theta | 219510_at |
| 200 | | RECQL4 | RecQ protein-like 4 | 213520_at |
| 201 | | NEIL3 | nei endonuclease VIII-like 3 | 219502_at |
| 202 | | RAD51AP1 | RAD51 associated protein 1 | 204146_at |
| 203 | | RAD54L | RAD54-like | 204558_at |
| 204 | | BRCA1 | breast cancer 1, early onset | 204531_s_at |
| 205 | | FANCL | Fanconi anemia, complementation group L | 218397_at |
| 206 | | WSB2 | WD repeat and SOCS box-containing 2 | 213734_at |
| 207 | | HTATIP2 | HIV-1 Tat interactive protein 2, 30 kDa | 209448_at |
| 208 | | IKBKG | inhibitor of kappa light polypeptide gene enhancer | 209929_s_at |
| | | | in B-cells, kinase gamma | |
| 209 | | LST1 | leukocyte specific transcript 1 | 215633_x_at |
| 210 | | LST1 | leukocyte specific transcript 1 | 210629_x_at |
| 211 | | HLA-DRB1 | major histocompatibility complex, class II, DR beta 1 | 204670_x_at |
| 212 | | LST1 | leukocyte specific transcript 1 | 211582_x_at |
| 213 | | HLA-DRA | major histocompatibility complex, class II, DR | 210982_s_at |
| | | | alpha | |
| 214 | | HLA-DRB1 | major histocompatibility complex, class II, DR beta 1 | 209312_x_at |
| 215 | | CCNA2 | Cyclin A2 | 213226_at |
| 216 | | HLA-DRA | major histocompatibility complex, class II, DR | 208894_at |
| | | | alpha | |
| 217 | | HLA-DPA1 | major histocompatibility complex, class II, DP | 211991_s_at |
| | | | alpha 1 | |
| 218 | | HLA-DRB1 | major histocompatibility complex, class II, DR beta 1 | 215193_x_at |
| 219 | | HLA-DMA | major histocompatibility complex, class II, DM | 217478_s_at |
| | | | alpha | |
| 220 | | CCL19 | chemokine (C-C motif) ligand 19 | 210072_at |
| 221 | | HLA-E | major histocompatibility complex, class I, E | 200904_at |
| 222 | | LST1 | leukocyte specific transcript 1 | 211581_x_at |
| 223 | | HLA-DQB1 | major histocompatibility complex, class II, DQ | 209823_x_at |
| | | | beta 1 | |
| 224 | | CXCL3 | chemokine (C—X—C motif) ligand 3 | 207850_at |
| 225 | | HLA-DRB1 | Major histocompatibility complex, class II, DR beta 3 | 208306_x_at |
| 226 | | STAT5A | signal transducer and activator of transcription 5A | 203010_at |
| 227 | | HLA-E | major histocompatibility complex, class I, E | 200905_x_at |
| 228 | | ARHGDIB | Rho GDP dissociation inhibitor (GDI) beta | 201288_at |
| 229 | | CD1E | CD1E antigen, e polypeptide | 215784_at |
| 230 | | CR2 | complement component (3d/Epstein Barr virus) | 205544_s_at |
| | | | receptor 2 | |
| 231 | | IGH | immunoglobulin heavy constant gamma 1 (G1m | 211430_s_at |
| | | | marker) | |
| 232 | | HLA-E | major histocompatibility complex, class I, E | 217456_x_at |
| 233 | | HLA-DPB1 | major histocompatibility complex, class II, DP beta 1 | 201137_s_at |
| 234 | | HLA-G | HLA-G histocompatibility antigen, class I, G | 211529_x_at |
| 235 | | IGJ | Immunoglobulin J polypeptide | 212592_at |
| 236 | | CXCL1 | chemokine (C—X—C motif) ligand 1 | 204470_at |
| 237 | | CXCL12 | chemokine (C—X—C motif) ligand 12 | 209687_at |
| 238 | | HLA-DOB | major histocompatibility complex, class II, DO | 205671_s_at |
| | | | beta | |
| 239 | | GBP2 | guanylate binding protein 2, interferon-inducible | 202748_at |
| 240 | | C3 | complement component 3 | 217767_at |
| 241 | | HLA-C | major histocompatibility complex, class I, C | 211799_x_at |
| 242 | | IFITM3 | interferon induced transmembrane protein 3 (1-8 U) | 212203_x_at |
| 243 | | CXCL12 | chemokine (C—X—C motif) ligand 12 | 203666_at |
| 244 | | AZGP1 | alpha-2-glycoprotein 1, zinc | 217014_s_at |
| 245 | | HLA-B | major histocompatibility complex, class I, B | 211911_x_at |
| 246 | | HLA-G | HLA-G histocompatibility antigen, class I, G | 210514_x_at |
| 247 | | IL2RG | interleukin 2 receptor, gamma | 204116_at |
| 248 | | CD74 | CD74 antigen | 209619_at |
| 249 | | HLA-B | major histocompatibility complex, class I, B | 208729_x_at |
| 250 | | MBP | myelin basic protein | 207323_s_at |
| 251 | | HLA-DQA1 /// | major histocompatibility complex, class II, DQ | 212671_s_at |
| | | HLA-DQA2 | alpha 1 | |
| 252 | | HLA-G | HLA-G histocompatibility antigen, class I, G | 211528_x_at |
| 253 | | CHUK | conserved helix-loop-helix ubiquitous kinase | 209666_s_at |
| 254 | | TNFRSF17 | tumor necrosis factor receptor superfamily, | 206641_at |
| | | | member 17 | |
| 255 | | FCER1A | Fc fragment of IgE, high affinity I, receptor for; | 211734_s_at |
| | | | alpha polypeptide | |
| 256 | | HLA-F | major histocompatibility complex, class I, F | 204806_x_at |
| 257 | | HLA-DRB4 | major histocompatibility complex, class II, DR beta 4 | 215669_at |
| 258 | | HFE | hemochromatosis | 206086_x_at |
| 259 | | C7 | complement component 7 | 202992_at |
| 260 | | CXCL5 | chemokine (C—X—C motif) ligand 5 | 214974_x_at |
| 261 | | RPL3 | ribosomal protein L3 | 211666_x_at |
| 262 | | RPS9 | ribosomal protein S9 | 217747_s_at |
| 263 | | RPL5 | ribosomal protein L5 | 200937_s_at |
| 264 | | RPS6 | ribosomal protein S6 | 200081_s_at |
| 265 | | EIF4B | eukaryotic translation initiation factor 4B | 211938_at |
| 266 | | RPS5 | ribosomal protein S5 | 200024_at |
| 267 | | EIF3S4 | eukaryotic translation initiation factor 3, subunit 4 | 208887_at |
| | | | delta, 44 kDa | |
| 268 | | RPL35A | ribosomal protein L35a | 213687_s_at |
| 269 | | RPL10A | ribosomal protein L10a | 200036_s_at |
| 270 | | RPL29 | ribosomal protein L29 | 200823_x_at |
| 271 | | RPL22 | ribosomal protein L22 | 220960_x_at |
| 272 | | RPL4 | ribosomal protein L4 | 211710_x_at |
| 273 | | MTA1 | metastasis associated 1 | 202247_s_at |
| 274 | | EIF3S7 | eukaryotic translation initiation factor 3, subunit 7 | 200005_at |
| | | | zeta, 66/67 kDa | |
| 275 | | RPL24 | ribosomal protein L24 | 200013_at |
| 276 | | RPL22 | ribosomal protein L22 | 221726_at |
| 277 | | RPS16 | ribosomal protein S16 | 201258_at |
| 278 | | EIF2C2 | Eukaryotic translation initiation factor 2C, 2 | 213310_at |
| 279 | | RPL14 | ribosomal protein L14 | 200074_s_at |
| 280 | | RPL18A | ribosomal protein L18a | 200869_at |
| 281 | | MRPL24 | mitochondrial ribosomal protein L24 | 218270_at |
| 282 | | MRPL9 | mitochondrial ribosomal protein L9 | 209609_s_at |
| 283 | | RPS6 | ribosomal protein S6 | 201254_x_at |
| 284 | | RPL4 | ribosomal protein L4 | 201154_x_at |
| 285 | | RPL11 | Ribosomal protein L11 | 200010_at |
| 286 | | PABPC4 | poly(A) binding protein, cytoplasmic 4 (inducible | 201064_s_at |
| | | | form) | |
| 287 | | RPL18 | ribosomal protein L18 | 200022_at |
| 288 | | KIAA0256 | KIAA0256 gene product | 212450_at |
| 289 | | RPS19 | ribosomal protein S19 | 213414_s_at |
| 290 | | RPS2 | Ribosomal protein S2 | 221798_x_at |
| 291 | | EIF4B | eukaryotic translation initiation factor 4B | 211937_at |
| 292 | | EIF3S1 | eukaryotic translation initiation factor 3, subunit 1 | 208264_s_at |
| | | | alpha, 35 kDa | |
| 293 | | RPL21 | ribosomal protein L21 | 200012_x_at |
| 294 | | RPS8 | ribosomal protein S8 | 200858_s_at |
| 295 | | RPS6 | ribosomal protein S6 | 209134_s_at |
| 296 | | RPL39 | ribosomal protein L39 | 208695_s_at |
| 297 | | ORC6L | origin recognition complex, subunit 6 homolog-like | 219105_x_at |
| 298 | | RRM2 | ribonucleotide reductase M2 polypeptide | 201890_at |
| 299 | | Pfs2 | DNA replication complex GINS protein PSF2 | 221521_s_at |
| 300 | | RRM2 | ribonucleotide reductase M2 polypeptide | 209773_s_at |
| 301 | | NFIB | Nuclear factor I/B | 213033_s_at |
| 302 | | FEN1 | flap structure-specific endonuclease 1 | 204767_s_at |
| 303 | | RFC3 | replication factor C (activator 1) 3, 38 kDa | 204127_at |
| 304 | | NAP1L1 | nucleosome assembly protein 1-like 1 | 208752_x_at |
| 305 | | TCL1B | T-cell leukemia/lymphoma 1B | 206413_s_at |
| 306 | | PIAS3 | protein inhibitor of activated STAT, 3 | 203035_s_at |
| 307 | | BIRC5 | baculoviral IAP repeat-containing 5 (survivin) | 202095_s_at |
| 308 | | JTB | jumping translocation breakpoint | 210434_x_at |
| 309 | | WHSC1 | Wolf-Hirschhorn syndrome candidate 1 | 209054_s_at |
| 310 | | JTB | jumping translocation breakpoint | 200048_s_at |
| 311 | | PTTG1 | pituitary tumor-transforming 1 | 203554_x_at |
| 312 | | ABCB6 | ATP-binding cassette, sub-family B (MDR/TAP), | 203192_at |
| | | | member 6 | |
| 313 | | GPR56 | G protein-coupled receptor 56 | 212070_at |
| 314 | | HDHD3 | haloacid dehalogenase-like hydrolase domain | 221256_s_at |
| | | | containing 3 | |
| 315 | | PDHX | pyruvate dehydrogenase complex, component X | 203067_at |
| 316 | | ATP9A | ATPase, Class II, type 9A | 212062_at |
| 317 | | LPGAT1 | lysophosphatidylglycerol acyltransferase 1 | 202651_at |
| 318 | | PSAT1 | phosphoserine aminotransferase 1 | 220892_s_at |
| 319 | | GALNS | galactosamine (N-acetyl)-6-sulfate sulfatase | 206335_at |
| 320 | | GFPT1 | glutamine-fructose-6-phosphate transaminase 1 | 202722_s_at |
| 321 | | ACACB | acetyl-Coenzyme A carboxylase beta | 221928_at |
| 322 | | FLJ21963 | FLJ21963 protein | 219616_at |
| 323 | | PFKFB3 | 6-phosphofructo-2-kinase/fructose-2,6- | 202464_s_at |
| | | | biphosphatase 3 | |
| 324 | | SCLY | selenocysteine lyase | 59705_at |
| 325 | | RDH11 | retinol dehydrogenase 11 | 217776_at |
| 326 | | PECI | peroxisomal D3,D2-enoyl-CoA isomerase | 218025_s_at |
| 327 | | ATP2C1 | ATPase, Ca++ transporting, type 2C, member 1 | 209935_at |
| 328 | | GSTP1 | glutathione S-transferase pi | 200824_at |
| 329 | | INSIG1 | insulin induced gene 1 | 201626_at |
| 330 | | SH2D1A | SH2 domain protein 1A, Duncan's disease | 210116_at |
| 331 | | CCR2 | chemokine (C-C motif) receptor 2 | 206978_at |
| 332 | | — | — | 211567_at |
| 333 | | GNLY | granulysin | 205495_s_at |
| 334 | | RALA | v-ral simian leukemia viral oncogene homolog A | 214435_x_at |
| | | | (ras related) | |
| 335 | | CCR7 | chemokine (C-C motif) receptor 7 | 206337_at |
| 336 | | SOCS5 | suppressor of cytokine signaling 5 | 209648_x_at |
| 337 | | SOCS5 | suppressor of cytokine signaling 5 | 208127_s_at |
| 338 | | NDN | necdin homolog (mouse) | 209550_at |
| 339 | | IGFBP7 | insulin-like growth factor binding protein 7 | 201162_at |
| 340 | | MAC30 | hypothetical protein MAC30 | 212279_at |
| 341 | | SOCS1 | suppressor of cytokine signaling 1 | 213337_s_at |
| 342 | | IGFBP7 | insulin-like growth factor binding protein 7 | 213910_at |
| 343 | | MORF4L1 | mortality factor 4 like 1 | 217982_s_at |
| 344 | | HTRA1 | HtrA serine peptidase 1 | 201185_at |
| 345 | | CTGF | connective tissue growth factor | 209101_at |
| 346 | | NEDD9 | neural precursor cell expressed, developmentally | 202149_at |
| | | | down-regulated 9 | |
| 347 | | IGFBP7 | insulin-like growth factor binding protein 7 | 201163_s_at |
| 348 | | ESM1 | endothelial cell-specific molecule 1 | 208394_x_at |
| 349 | | OGFR | opioid growth factor receptor | 211513_s_at |
| 350 | | OGFR | opioid growth factor receptor | 211512_s_at |
| 351 | | RGS4 | regulator of G-protein signalling 4 | 204337_at |
| 352 | | RGS16 | regulator of G-protein signalling 16 | 209324_s_at |
| 353 | | RGS3 | regulator of G-protein signalling 3 | 220300_at |
| 354 | | RGS2 | regulator of G-protein signalling 2, 24 kDa | 202388_at |
| 355 | | GRK5 | G protein-coupled receptor kinase 5 | 204396_s_at |
| 356 | | COL2A1 | collagen, type II, alpha 1 | 217404_s_at |
| 357 | | SHOX2 | short stature homeobox 2 | 210135_s_at |
| 358 | | COL10A1 | collagen, type X, alpha 1 | 205941_s_at |
| 359 | | AEBP1 | AE binding protein 1 | 201792_at |
| 360 | | MATN3 | matrilin 3 | 206091_at |
| 361 | | SHOX2 | short stature homeobox 2 | 208443_x_at |
| 362 | | TWIST1 | twist homolog 1(Drosophila) | 213943_at |
| 363 | | ANKH | ankylosis, progressive homolog (mouse) | 220076_at |
| 364 | | ANXA2 | annexin A2 | 210427_x_at |
| 365 | | POSTN | periostin, osteoblast specific factor | 210809_s_at |
| 366 | | FGFR1 | fibroblast growth factor receptor 1 | 210973_s_at |
| 367 | | ANXA2 | annexin A2 | 213503_x_at |
| 368 | | CDC42BPA | CDC42 binding protein kinase alpha (DMPK-like) | 213595_s_at |
| 369 | | MAPKAPK2 | mitogen-activated protein kinase-activated protein | 215050_x_at |
| | | | kinase 2 | |
| 370 | | PAK2 | p21 (CDKN1A)-activated kinase 2 | 208875_s_at |
| 371 | | TAF1 | TAF1 RNA polymerase II, TATA box binding | 216711_s_at |
| | | | protein (TBP)-associated factor | |
| 372 | | PDGFRA | platelet-derived growth factor receptor, alpha | 203131_at |
| | | | polypeptide | |
| 373 | | CLK1 | CDC-like kinase 1 | 214683_s_at |
| 374 | | ADRBK1 | adrenergic, beta, receptor kinase 1 | 201401_s_at |
| 375 | | MAP4K5 | mitogen-activated protein kinase kinase kinase | 203552_at |
| | | | kinase 5 | |
| 376 | | PRKD1 | protein kinase D1 | 205880_at |
| 377 | | PRKAR1A | protein kinase, cAMP-dependent, regulatory, type | 200604_s_at |
| | | | I, alpha | |
| 378 | | PCTK1 | PCTAIRE protein kinase 1 | 207239_s_at |
| 379 | | PTK9 | PTK9 protein tyrosine kinase 9 | 214007_s_at |
| 380 | | NEK7 | NIMA (never in mitosis gene a)-related kinase 7 | 212530_at |
| 381 | | PIK3R4 | phosphoinositide-3-kinase, regulatory subunit 4, | 212740_at |
| | | | p150 | |
| 382 | | CDC42BPA | CDC42 binding protein kinase alpha (DMPK-like) | 215296_at |
| 383 | | MAPKAPK2 | mitogen-activated protein kinase-activated protein | 201461_s_at |
| | | | kinase 2 | |
| 384 | | MAP2K3 | mitogen-activated protein kinase kinase 3 | 207667_s_at |
| 385 | | PRPF4B | PRP4 pre-mRNA processing factor 4 homolog B | 202127_at |
| | | | (yeast) | |
| 386 | | BMP2K | BMP2 inducible kinase | 59644_at |
| 387 | | PRKACG | protein kinase, cAMP-dependent, catalytic, | 207228_at |
| | | | gamma | |
| 388 | | MAP2K2 | mitogen-activated protein kinase kinase 2 | 213490_s_at |
| 389 | | MET | met proto-oncogene (hepatocyte growth factor | 211599_x_at |
| | | | receptor) | |
| 390 | | CASK | calcium/calmodulin-dependent serine protein | 211208_s_at |
| | | | kinase (MAGUK family) | |
| 391 | | ROR2 | receptor tyrosine kinase-like orphan receptor 2 | 205578_at |
| 392 | | MAPK10 | mitogen-activated protein kinase 10 | 204813_at |
| 393 | | PCTK1 | PCTAIRE protein kinase 1 | 208824_x_at |
| 394 | | RND3 | Rho family GTPase 3 | 212724_at |
| 395 | | PLEKHC1 | pleckstrin homology domain containing, family C | 209210_s_at |
| | | | member 1 | |
| 396 | | SPOCK | sparc/osteonectin, cwcv and kazal-like domains | 202363_at |
| | | | proteoglycan (testican) | |
| 397 | | TGFB1I1 | transforming growth factor beta 1 induced | 209651_at |
| | | | transcript 1 | |
| 398 | | LAMB1 | laminin, beta 1 | 201505_at |
| 399 | | LAMC1 | laminin, gamma 1 (formerly LAMB2) | 200771_at |
| 400 | | ADAM12 | ADAM metallopeptidase domain 12 (meltrin | 213790_at |
| | | | alpha) | |
| 401 | | THBS2 | thrombospondin 2 | 203083_at |
| 402 | | HNT | neurotrimin | 222020_s_at |
| 403 | | CDH6 | cadherin 6, type 2, K-cadherin (fetal kidney) | 205532_s_at |
| 404 | | MLLT4 | myeloid/lymphoid or mixed-lineage leukemia; | 215904_at |
| | | | translocated to, 4 | |
| 405 | | CLSTN1 | calsyntenin 1 | 201561_s_at |
| 406 | | CDH5 | cadherin 5, type 2, VE-cadherin (vascular | 204677_at |
| | | | epithelium) | |
| 407 | | PLEKHC1 | pleckstrin homology domain containing, family C | 214212_x_at |
| | | | (with FERM domain) member 1 | |
| 408 | | PPFIBP1 | PTPRF interacting protein, binding protein 1 (liprin | 214375_at |
| | | | beta 1) | |
| 409 | | SRPX | sushi-repeat-containing protein, X-linked | 204955_at |
| 410 | | PKP3 | plakophilin 3 | 209873_s_at |
| 411 | | ITGB3BP | integrin beta 3 binding protein (beta3-endonexin) | 205176_s_at |
| 412 | | ADRM1 | adhesion regulating molecule 1 | 201281_at |
| 413 | | NCAM1 | neural cell adhesion molecule 1 | 212843_at |
| 414 | | PCDH17 | protocadherin 17 | 205656_at |
| 415 | | COL6A3 | collagen, type VI, alpha 3 | 201438_at |
| 416 | | PLXNC1 | plexin C1 | 213241_at |
| 417 | | COL5A3 | collagen, type V, alpha 3 | 218975_at |
| 418 | | SLC2A3 | solute carrier family 2, member 3 | 202499_s_at |
| 419 | | FUT3 | fucosyltransferase 3 | 216010_x_at |
| 420 | | SLC3A1 | solute carrier family 3, member 1 | 205799_s_at |
| 421 | | HEXA | hexosaminidase A (alpha polypeptide) | 201765_s_at |
| 422 | | SFRS11 | splicing factor, arginine/serine-rich 11 | 200686_s_at |
| 423 | | CDC40 | cell division cycle 40 homolog (yeast) | 203376_at |
| 424 | | PRPF4 | PRP4 pre-mRNA processing factor 4 homolog | 209162_s_at |
| | | | (yeast) | |
| 425 | | SFRS9 | splicing factor, arginine/serine-rich 9 | 201698_s_at |
| 426 | | SFRS11 | splicing factor, arginine/serine-rich 11 | 200685_at |
| 427 | | PRPF18 | PRP18 pre-mRNA processing factor 18 homolog | 221546_at |
| | | | (yeast) | |
| 428 | | DHX15 | DEAH (Asp-Glu-Ala-His) box polypeptide 15 | 201385_at |
| 429 | | THOC1 | THO complex 1 | 204064_at |
| 430 | | SFPQ | Splicing factor proline/glutamine-rich | 214016_s_at |
| 431 | | LSM8 | LSM8 homolog, U6 small nuclear RNA associated | 219119_at |
| 432 | | EDNRA | endothelin receptor type A | 204464_s_at |
| 433 | | ELK3 | ELK3, ETS-domain protein (SRF accessory | 221773_at |
| | | | protein 2) | |
| 434 | | IDE | insulin-degrading enzyme | 203328_x_at |
| 435 | | PRKAB1 | protein kinase, AMP-activated, beta 1 non- | 201835_s_at |
| | | | catalytic subunit | |
| 436 | | IDE | insulin-degrading enzyme | 217496_s_at |
| 437 | | PTPN11 | protein tyrosine phosphatase, non-receptor type | 209895_at |
| | | | 11 | |
| 438 | | PTPN1 | protein tyrosine phosphatase, non-receptor type 1 | 202716_at |
| 439 | | ARFRP1 | ADP-ribosylation factor related protein 1 | 215984_s_at |
| 440 | | CYTL1 | cytokine-like 1 | 219837_s_at |
| 441 | | GNRH1 | gonadotropin-releasing hormone 1 | 207987_s_at |
| 442 | | GNG11 | guanine nucleotide binding protein (G protein), | 204115_at |
| | | | gamma 11 | |
| 443 | | CDC42SE1 | CDC42 small effector 1 | 218157_x_at |
| 444 | | PDE4B | phosphodiesterase 4B, cAMP-specific | 211302_s_at |
| 445 | | IPO8 | importin 8 | 205701_at |
| 446 | | IQGAP1 | IQ motif containing GTPase activating protein 1 | 213446_s_at |
| 447 | | CASP8AP2 | CASP8 associated protein 2 | 222201_s_at |
| 448 | | GTF2I | general transcription factor II, I | 201065_s_at |
| 449 | | CD40 | CD40 antigen (TNF receptor superfamily member | 35150_at |
| | | | 5) | |
| 450 | | GNG12 | guanine nucleotide binding protein (G protein), | 212294_at |
| | | | gamma 12 | |
| 451 | | MARCKSL1 | MARCKS-like 1 | 200644_at |
| 452 | | CHRNA3 | cholinergic receptor, nicotinic, alpha polypeptide 3 | 210221_at |
| 453 | | KIR2DL4 | killer cell immunoglobulin-like receptor, two | 211245_x_at |
| | | | domains, long cytoplasmic tail, 4 | |
| 454 | | KIR2DL4 | killer cell immunoglobulin-like receptor, two | 211242_x_at |
| | | | domains, long cytoplasmic tail, 4 | |
| 455 | | OR3A2 | olfactory receptor, family 3, subfamily A, member 2 | 221386_at |
| 456 | | TXNIP | thioredoxin interacting protein | 201008_s_at |
| 457 | | COPS2 | COP9 constitutive photomorphogenic homolog | 202467_s_at |
| | | | subunit 2 (Arabidopsis) | |
| 458 | | EPOR | erythropoietin receptor | 396_f_at |
| 459 | | KHDRBS1 | KH domain containing, RNA binding, signal | 201488_x_at |
| | | | transduction associated 1 | |
| 460 | | WDR68 | WD repeat domain 68 | 221745_at |
| 461 | | NR2F1 | Nuclear receptor subfamily 2, group F, member 1 | 209505_at |
| 462 | | — | — | 213401_s_at |
| 463 | | ARL2BP | ADP-ribosylation factor-like 2 binding protein | 202091_at |
| 464 | | TXNIP | thioredoxin interacting protein | 201009_s_at |
| 465 | | MPP2 | membrane protein, palmitoylated 2 (MAGUK p55 | 213270_at |
| | | | subfamily member 2) | |
| 466 | | MCC | mutated in colorectal cancers | 206132_at |
| 467 | | MAPK9 | mitogen-activated protein kinase 9 | 203218_at |
| 468 | | PAK4 | p21(CDKN1A)-activated kinase 4 | 33814_at |
| 469 | | SMAD2 | SMAD, mothers against DPP homolog 2 | 203077_s_at |
| | | | (Drosophila) | |
| 470 | | DPYSL3 | dihydropyrimidinase-like 3 | 201431_s_at |
| 471 | | TLR4 | toll-like receptor 4 | 221060_s_at |
| 472 | | WIF1 | WNT inhibitory factor 1 | 204712_at |
| 473 | | LGALS3BP | lectin, galactoside-binding, soluble, 3 binding | 200923_at |
| | | | protein | |
| 474 | | APPL | adaptor protein containing pH domain, PTB | 218158_s_at |
| | | | domain and leucine zipper motif 1 | |
| 475 | | DRD5 | dopamine receptor D5 | 208486_at |
| 476 | | TRPC1 | transient receptor potential cation channel, | 205802_at |
| | | | subfamily C, member 1 | |
| 477 | | PKD2 | polycystic kidney disease 2 (autosomal dominant) | 203688_at |
| 478 | | TRPC1 | transient receptor potential cation channel, | 205803_s_at |
| | | | subfamily C, member 1 | |
| 479 | | ATP13A3 | ATPase type 13A3 | 212297_at |
| 480 | | TRPA1 | transient receptor potential cation channel, | 208349_at |
| | | | subfamily A, member 1 | |
| 481 | | SLC24A3 | solute carrier family 24 | 219090_at |
| | | | (sodium/potassium/calcium exchanger), member 3 | |
| 482 | | RNF19 | ring finger protein 19 | 220483_s_at |
| 483 | | LIPT1 | lipoyltransferase 1 | 205571_at |
| 484 | | RPN2 | ribophorin II | 208689_s_at |
| 485 | | RABGGTB | Rab geranylgeranyltransferase, beta subunit | 213704_at |
| 486 | | PDLIM2 | PDZ and LIM domain 2 (mystique) | 219165_at |
| 487 | | DLG3 | discs, large homolog 3 (neuroendocrine-dlg, | 212729_at |
| | | | Drosophila) | |
| 488 | | TNS1 | tensin 1 | 221748_s_at |
| 489 | | SHANK2 | SH3 and multiple ankyrin repeat domains 2 | 215829_at |
| 490 | | CIT | citron (rho-interacting, serine/threonine kinase 21) | 212801_at |
| 491 | | CRK | v-crk sarcoma virus CT10 oncogene homolog | 202226_s_at |
| | | | (avian) | |
| 492 | | RIN2 | Ras and Rab interactor 2 | 209684_at |
| 493 | | DLG3 | discs, large homolog 3 (neuroendocrine-dlg, | 207732_s_at |
| | | | Drosophila) | |
| 494 | | PDLIM7 | PDZ and LIM domain 7 (enigma) | 203370_s_at |
| 495 | | SNX3 | sorting nexin 3 | 213545_x_at |
| 496 | | SNX3 | sorting nexin 3 | 210648_x_at |
| 497 | | SNX2 | sorting nexin 2 | 202114_at |
| 498 | | SNX24 | sorting nexing 24 | 218705_s_at |
| 499 | | NCF4 | neutrophil cytosolic factor 4, 40 kDa | 205147_x_at |
| 500 | | PSEN1 | presenilin 1 | 207782_s_at |
| 501 | | SNX3 | sorting nexin 3 | 200067_x_at |
| 502 | | PIK3R2 | phosphoinositide-3-kinase, regulatory subunit 2 | 207105_s_at |
| | | | (p85 beta) | |
| 503 | | STAT2 | signal transducer and activator of transcription 2, | 205170_at |
| | | | 113 kDa | |
| 504 | | TRAF3IP2 | TRAF3 interacting protein 2 | 215411_s_at |
| 505 | | RIN3 | Ras and Rab interactor 3 | 219457_s_at |
| 506 | | PARD3 | par-3 partitioning defective 3 homolog (C. elegans) | 221526_x_at |
| 507 | | TAX1BP3 | Tax1 binding protein 3 | 209154_at |
| 508 | | TRAF3IP2 | TRAF3 interacting protein 2 | 202987_at |
| 509 | | HNRPA1 | heterogeneous nuclear ribonucleoprotein A1 | 222040_at |
| 510 | | HNRPR | heterogeneous nuclear ribonucleoprotein R | 208765_s_at |
| 511 | | — | — | 221919_at |
| 512 | | SIP1 | survival of motor neuron protein interacting protein 1 | 205063_at |
| 513 | | SRRM1 | serine/arginine repetitive matrix 1 | 201224_s_at |
| 514 | | IVNS1ABP | influenza virus NS1A binding protein | 201362_at |
| 515 | | DNM3 | dynamin 3 | 209839_at |
| 516 | | FLJ14107 | hypothetical protein FLJ14107 | 207287_at |
| 517 | | ZFPM2 | zinc finger protein, multitype 2 | 219778_at |
| 518 | | FOXO1A | forkhead box O1A | 202724_s_at |
| 519 | | SMARCA2 | SWI/SNF related, matrix associated, actin | 212257_s_at |
| | | | dependent regulator of chromatin, subfamily a, | |
| | | | member 2 | |
| 520 | | NFYC | nuclear transcription factor Y, gamma | 202216_x_at |
| 521 | | CRSP9 | cofactor required for Sp1 transcriptional activation, | 204349_at |
| | | | subunit 9, 33 kDa | |
| 522 | | HOXC6 | homeo box C6 | 206858_s_at |
| 523 | | TCF4 | Transcription factor 4 | 213891_s_at |
| 524 | | SMARCC1 | SWI/SNF related, matrix associated, actin | 201073_s_at |
| | | | dependent regulator of chromatin, subfamily c, | |
| | | | member 1 | |
| 525 | | SMARCA5 | SWI/SNF related, matrix associated, actin | 213251_at |
| | | | dependent regulator of chromatin, subfamily a, | |
| | | | member 5 | |
| 526 | | ID4 | Inhibitor of DNA binding 4, dominant negative | 209292_at |
| | | | helix-loop-helix protein | |
| 527 | | FOS | v-fos FBJ murine osteosarcoma viral oncogene | 209189_at |
| | | | homolog | |
| 528 | | ZNF161 | zinc finger protein 161 | 202172_at |
| 529 | | PDGFB | platelet-derived growth factor beta polypeptide | 216061_x_at |
| 530 | | MTCP1 | mature T-cell proliferation 1 | 205106_at |
| 531 | | HYPE | Huntingtin interacting protein E | 219910_at |
| 532 | | E2F4 | E2F transcription factor 4, p107/p130-binding | 38707_r_at |
| 533 | | PPM1D | protein phosphatase 1D magnesium-dependent, | 204566_at |
| | | | delta isoform | |
| 534 | | CCND3 | cyclin D3 | 201700_at |
| 535 | | MAPRE1 | microtubule-associated protein, RP/EB family, | 200712_s_at |
| | | | member 1 | |
| 536 | | SPHAR | S-phase response (cyclin-related) | 206272_at |
| 537 | | PICALM | phosphatidylinositol binding clathrin assembly | 212511_at |
| | | | protein | |
| 538 | | DARS | aspartyl-tRNA synthetase | 201624_at |
| 539 | | VAMP4 | vesicle-associated membrane protein 4 | 213480_at |
| 540 | | TAPBP | TAP binding protein (tapasin) | 208829_at |
| 541 | | RANBP9 | RAN binding protein 9 | 216125_s_at |
| 542 | | DAG1 | dystroglycan 1 (dystrophin-associated | 212128_s_at |
| | | | glycoprotein 1) | |
| 543 | | EPRS | glutamyl-prolyl-tRNA synthetase | 200841_s_at |
| 544 | | RPL26L1 | ribosomal protein L26-like 1 | 218830_at |
| 545 | | RPL34 | ribosomal protein L34 | 200026_at |
| 546 | | RPL31 | ribosomal protein L31 | 200963_x_at |
| 547 | | MRPS18A | mitochondrial ribosomal protein S18A | 221693_s_at |
| 548 | | RPL36 | ribosomal protein L36 | 219762_s_at |
| 549 | | RPL31 | ribosomal protein L31 | 221593_s_at |
| 550 | | RPS25 | ribosomal protein S25 | 200091_s_at |
| 551 | | EIF3S2 | eukaryotic translation initiation factor 3, subunit 2 | 208756_at |
| | | | beta, 36 kDa | |
| 552 | | MRPL33 | mitochondrial ribosomal protein L33 | 203781_at |
| 553 | | NAG | neuroblastoma-amplified protein | 202926_at |
| 554 | | RPL24 | ribosomal protein L24 | 214143_x_at |
| 555 | | RCC1 | regulator of chromosome condensation 1 | 215747_s_at |
| 556 | | CUL5 | cullin 5 | 203531_at |
| 557 | | RBBP4 | retinoblastoma binding protein 4 | 217301_x_at |
| 558 | | ATR | ataxia telangiectasia and Rad3 related | 209903_s_at |
| 559 | | PARD6A | par-6 partitioning defective 6 homolog alpha | 205245_at |
| | | | (C. elegans) | |
| 560 | | 38967 | septin 7 | 213151_s_at |
| 561 | | RBL2 | retinoblastoma-like 2 (p130) | 212332_at |
| 562 | | NOLC1 | nucleolar and coiled-body phosphoprotein 1 | 205895_s_at |
| 563 | | CCNT1 | cyclin T1 | 206967_at |
| 564 | NM_006845 | | mitotic centromere-associated kinesin mitotic | 209408 |
| | | | centromere-associated kinesin | |

Additional sequences

SEQ ID NO: 501

tctttcccccttttaatttgtgatgtcacttgaccccatttatgtgtagg
agcactacaccattggtttccaatactgcacacataagatacatacttgt
gtgcagaaagtatcttcctccaggcttgtaatacccttcacatggaagat
taatgagggaaatctttatattctgtataaaaacaaaagcaaatttatat
actaaaatcatttgtctaaaaatttaagttgttttcaaataaaaattaaa
atgcatttctgatatgcaaaaaaaaaaaaaaaaaaaaaaaaaaannnnnn
nnnnannanngannanntaagtcacttgttgagagggattatttactaat
tatatacttctcattcctgtaactccattccctttaaacagtggtgatat
caaatatacttccatccattgaatggggtatttttaacaacaacaaaagt
gatatactaaaaaatgtattgcttaaggcttattgaatcattttgaagca
ctttgtgtatttgaaaactgctttataatctcattta

SEQ ID NO: 502

tctctccatgttgggggtcctaactcccccaccccatatctacgtgtcct
ccgggcattgccctctccatggctctggtcaccctgaccctctgccctgc
ccaccgcaggtcccccggggtcccggaagccccttctggctgcacctgcc
atgtttacagagggcccctgggctgcgcggccccagcctgggcaccctga
tttttaagccatagacctggggtcagggcaggaaggaacttcactctgct
gcttccgagaacctcggccgtgacattcggggccgggcgggacccgcccc
acagactccaacttcccctccaaaccccgaagtgaaacccgccaccgggt
taccccacaagggggccgctgcgagaagttcacccacccccgaaaaaata
attaaactcgcaggccaggcacg

SEQ ID NO: 503

tcccttccaagctgtgttaactgttcaaactcaggcctgtgtgactccat
tggggtgagaggtgaaagcataacatgggtacagaggggacaacaatgaa
tcagaacagatgctgagccataggtctaaataggatcctggaggctgcct
gctgtgctgggaggtataggggtcctgggggcaggccagggcagttgaca
ggtacttggagggctcagggcagtggcttctttccagtatggaaggattt
caacattttaatagttggttaggctaaactggtgcatactggcattggcc
ttggtggggagcacagacacaggataggactccatttctttcttccattc
cttcatgtctaggataacttgctttcttctttcctttactcctggctcaa
gccctgaatttcttcttttcctgcaggggttgagagctttctgccttagc
ctaccatgtgaaactctaccctgaag

SEQ ID NO: 504

cagaacactcatgtctacagctggcccaagaataaaaaaaacatcctgct
gcggctgctgagagaggaagagtatgtggctcctccacgggggcctctng
cccacccttncaggtggttcccttgtgacaccgttcatccccagatcact
gaggccaggccatgtttggggccttgttctgacagcattctggctgaggc
tggtcggtagcactcctggctggtttttttctgttcctccccgagaggcc
ctctggcccccaggaaacctgttgtgcagagctcttccccggagacctcc
acacaccctggctttgaagtggagtctgtgactgctctgcattctctgct
tttaaaaaaaccattgcaggtgccagtgtcccatatgttccnnctgacag
tttgatgtgnccattctgggcctctcagtgcttagcnagtagataatngt
angggatgtggcagcaaatggnaatgactacaaacactctnctatcaatc
acttcaggctacttttatgagttagccagatgcttgtgtatcctcagacc
aaactg

SEQ ID NO: 505

gaaagccttttgtccaaatatggaacttgaatgatatggcaaaattagaa
atgcaattttagaagtaattacactgttgtgtaaatggccacctcttttg
aagtctttgctacattgcttataaaacactgagttgaacatgagaaagcc
ttttgtctgcagctgtacttttcaactggacatgaaccatgtacttttat
ggcacgtagatattcacatcaaatttctgatttgcagaccgattttattt
ttagttaacaaataagcnttatcnaaatgtggcttttgaactaaagcgct
tttaattaaggagttataacagcatgttattttgagtagctgttactaaa
atctgttgtgatggaacaatttggagtgagcatctgatatcagagataaa
gagagaagcatgcagtgagcatctggaagttcttgtaaaaaaaaaaacaa
attaaacattctcatttgaatgcatttaaaatttttttaaattgccaatt
cctaagctttttctttgttagttg

SEQ ID NO: 506

atcagtgattcagccgactgctctttgagtccagatgttgatccagttct
tgcttttcaacgagaaggatttggacgtcagagtatgtcagaaaaacgca
caaagcaattttcagatgccagtcaattggatttcgttaaaacacgaaaa
tcaaaaagcatggatttaggtatagctgacgagactaaactcaatacagt
ggatgaccagaaagcaggttctcccagcagagatgtgggtccttccctgg
gtctgaagaagtcaagctcnttggagagtctgcagaccgcagttgccgag
gtgactttgaatggggatattcctttccatcgtccacggccgcggataat
cagaggcaggggatgcaatgagagcttcagagctgccatcgacaaatctt
atgataaacccgcggtagatgatgatgatgaaggcatggagaccttggaa
gaagacacagaagaaagttcaagatcagggagagagtctgtatccacagc
cagtgatcagccttcccactctctggagagacaa

SEQ ID NO: 507

atgtttttatcgtactctttggagatgcccattctacttttgaatttagc
ttttactaattcgcatctggaagctcagcaagtgcacaagccttactttg
gttaccgtg

SEQ ID NO: 508

gtaagactttctgacatgtaacattagttccgtagttttgagacctggta
gaactgactttcatatttggataacctggaaaacacccaaacacaaactt
caagtcttctttctcttttttcattatcttttttagtctgaggtgacacc
atcattaaggattcgacacccgtttgtaaataaaatgacatcagcaatta
ctctgaaatgtttctagtttgcaaagatttagcaatgtgatgttattaac
ccttcctcccttcagagacctgtcctaagctctgaaccactcattccttc
cactcttcttaccccaggtggttgatgagcagtggtccctggtgt

SEQ ID NO: 509

cagcaaaagaatgccctgcgttcccaaagtaaaagaatgacaagctgtac
cttaaaccaaaacacttcgtaatctcatccaattgcaaaaagagttatta
gccaaccaggtattcccagtagtgacagtggatataactgtgtagtcatt
cacctctgcttatatgaatactttacaacctcttttgcct

SEQ ID NO: 510

tggatatggctaccctccagattactacggctatgaagattactatgatg
attactatggttatgattatcacgactatcgtggaggctatgaagatccc
tactacggctatgatgatggctatgcagtaagaggaagaggaggaggaag
gggagggcgaggtgctccaccaccaccaagggggaggggagcaccacctc
caagaggtagagctggctattcacagaggggggcacctttgggaccacca
agaggctctaggggtggcagagggggtcctgctcaacagcagagaggccg
tggttcccgtggatctcggggcaatcgtgggggcaatgtaggaggcaaga
gaaaggcagatgggtacaaccagcctgattccaagcgtcgtcagaccaac
aaccaacagaactggggttcccaacccatcgctcagcagccgcttcagca
aggtggtgactattctggtaac

SEQ ID NO: 511

gaacagattttacttacatccatatagttacttaaagtccagttttctgt
taaacatttttcttaatatattgagccaaaactagtccagttaagctgaa
cttggtttttctggagatgaattgttttaaattgacaccctattgatggc
tcccagttgaaggaagtgagcacattatttgtactgtgaatataaatttt
tgcccttttatttatcttcctttgacccatttccttaaaataatggctca
aagtaatagacttccccaaatggtggggggatgggtgggttattaatggg
aggtatggggggtttagcttgagatgggacttggtcttagagctagttct

SEQ ID NO: 512

aacaatgccaattcaagtacagatttcaacacatcttcaacactatgtga
agggttcacatcttaacctgtgcaattcagattgatactcagaatatggg
ttgatttgaatatctgaaatatcaatggaaaatcccactcagtttttgat
gaacagtttgaacagttttctgtaatcaagcagcttgcatagaaattgta
tgatgaaattttacataggttcttggtgctg

SEQ ID NO: 513

ctccccctcctaaacgaagagcatcaccatctccaccaccaaagcggcgg
gtctcccattctccacctcccaaacaaagaagctccccagtcaccaagag
acgttcaccttcattatcatccaagcataggaaagggtcttccccaagcc
gctctacccgggaggcccgatcaccacaaccaaacaaacggcattcgccc
tcaccacggcctcgagctcctcagacctcctcaagtcctccacccgttcg
aagaggagcgtcgtcatcaccccaaagaaggcagtccccgtctccaagta
ctaggcccattaggagagtctccaggactccggaacctaaaaagataaaa
aaggctgcttccccaagcccacagtctgtaagaagggtctcatcctcccg
atctgtctccgggtctcctgagccagcagctaaaaagcccccagcacctc
catcccccgtccagtctcagtcaccgtctacaaactggtcaccagctgta
ccggtc

SEQ ID NO: 514

gcaggaaatccttgcaccatgggattaatatccaattgctgcttgtacac
tcattcattactaaaagttttgagaaatttttttttccagtaatgagctt
aagaaatttgtggaaaataactcacctggcatcttacatctgaaataagg
aatgatataaggtttttttttctcacagaagatgaagcacacaggaacct
aatgggccaactgggatgaggtgactattctgagatgactattcagtggc
taacttgggttaggaagaaaataattaggtattttctccaaatgttcact
ggtactctgccactttatttctctcatctgttacacaaagaaccaccagg
aaagcaaatcagtttggttggtaactctgtaattcctaactatcactggt
ttggttctggactaaaactacattgacagattgaatttgcctaatatgat
gactgtttttaatatggatctgtatgtgttctattcagcccaagga

SEQ ID NO: 515

gagacttctcacttctggttggaggtttcacatatggctcaactcaagtc
attaatctctttttaatttttactcttgaattccttaaacttcgctcatt
atgaaatgttttaaaattatgacaaaaattactctgtctaaccacttgcc
ttgtctgctaccagtttgttaaaaattattccccccaaccagtaattcca
ccagtactacttgatttgtgttatatttcctatgtacatgtacagccttt
gttttgcttgcttgtctatttttactttcccttttttgggtcaaattttt
cttttgctttgtttgaagaaggaatatacagaagtaaaatcttgtcttct
ctgctgattctttaattaatatgagccggatactttccactgtcttcttg
gcactttcaggatttcttaatgctgatatatggactcttagaatggaatt
tttgaagaaaaatctcaaagcctgtatcgttct

SEQ ID NO: 516

ggctgtcagatggccttgagcggcaccaagtagaaaacgcgctcccaccc
ctgaccttctcctcagcttcattgtgagacctcaagttcctcagcttcca
ggatgatcaacctagctgaaaacctgaagtccctcccggtacaagtccaa
gcagtccccagccagggagaccaggtgttgtctgacatcccacacacatc
ggcacacttgggggattgcaaaagggaggaagggagccaaaggctagggc
cccggggttcagctaacactcagcacccctcccaaagagcgccccctgtg
tgttctggatctctagaggggtttggtttgggccaagtagtgcttagttt
taattttctctttctggaaataaatacttttaataagtaaagatgctgct
cagctgtcatatcctgcaaggttagaggaaagatgtgggccgtgcgcg

SEQ ID NO: 517

atacacatgctataagttcgccttaagatttcaattcttggataatcagg
ctctgtttgcactttatattttagcagatacagtctcttagtcactaggc
tttgcatttgtatgtagctgtatgtttccgtccattttcttaatcctgaa
cctgtatgttaaatgaagatggcaatttttttcttgtatagtacttgtat
tttctttcgctgatgcagctctgtctcaatttttaaacctttgctgttaa
atgcaatactttataaagaatgaacaaaattactggaagcagtattgtaa
gtaatgaggtagtattaatcagttttatcttttgaaaggcacagtctaaa
tcgaaaccctaaactcaatgctgcaagtatgaatttaattcatatataag
atctatttaaatataagagtagcaatactgcacctggtgatca

SEQ ID NO: 518

gagcagtaaatcaatggaacatcccaagaagaggataaggatgcttaaaa
tggaaatcattctccaacgatatacaaattggacttgttcaactgctgga
tatatgctaccaataaccccagccccaacttaaaattcttacattcaagc
tcctaagagttcttaatttataactaattttaaaagagaagtttcttttc
tggttttagtttgggaataatcattcattaaaaaaaatgtattgtggttt
atgcgaacagaccaacctggcattacagttggcctctccttgaggtgggc
acagcctggcagtgtggccaggggtggccatgtaagtcccatcaggacgt
agtcatgcctcctgcatttcgctacccgagtttagtaacagtgcagattc
cacgttcttgttccgatactctgagaagtgcctgatgttgatgtacttac
agacacaagaacaatctttgctataa

SEQ ID NO: 519

gcaaccacccatatatgtttcagcacattgaggaatcctttgctgaacac
ctaggctattcaaatggggtcatcaatggggctgaactgtatcgggcctc
agggaagtttgagctgcttgatcgtattctgccaaaattgagagcgacta
atcaccgagtgctgcttttctgccagatgacatctctcatgaccatcatg
gaggattattttgcttttcggaacttcctttacctacgccttgatggcac
caccaagtctgaagatcgtgctgctttgctgaagaaattcaatgaacctg
gatcccagtatttcattttcttgctgagcacaagagctggtggcctgggc
ttaaatcttcaggcagctgatacagtggtcatctttgacagcgactgg

SEQ ID NO: 520

gatcccggtgcagctgaatgccggccagctgcagtatatccgcttagccc
agcctgtatcaggcactcaagttgtgcagggacagatccagacacttgcc
accaatgctcaacagattacacagacagaggtccagcaaggacagcagca
gttcagccagttcacagatggacagcagctctaccagatccagcaagtca
ccatgcctgcgggccaggacctcgcccagcccatgttcatccagtcagcc
aaccagccctccgacgggcaggccccccaggtgaccggcgactgagggcc
tgagctggcaaggccaaggacacccaacacaatttttgccatacagcccc
aggcaatgggcacagccttcctccccagaggacccggccgacctcagcgc
ctcctgcaggctaggacactggtgcactacacc

SEQ ID NO: 521

ttttccttttgataatagcatcatatattagttcattttcttttggacag
tcttaagagaagtttcactaaaaatgtaaacagctttaatcttgactcca
aatttttcaattatgagatgtcataggcagtaatttcgctgtataacaag
catagacaaatgagtgtccctgcactaagaagaatcactttaaaaagcaa
agtgttagctgctgttgtatgggacattcctatgttttagagttgcagta
aaactttgatgataacctcaataatagcaaagtgg

SEQ ID NO: 522

ggaccctgaactcagactctacagattgccctccaagtgaggacttggct
cccccactccttcgacgcccccacccccgccccccgtgcagagagccggc
tcctgggcctgctggggcctctgctccagggcctcagggccggcctggca
gccggggagggccggagcggagggcgcgccttggccccacaccaaccccc
agggcctccccgcagtccctgcctagcccctctgccccagcaaatgccca
gcccaggcaaattgtatttaaagaatcctgggggtcattatggcatttta
caaactgtgaccgtttctgtgtgaagatttttagctgtatttgtggtctc
tgtatttatatttatgtttagcaccgtcagtgttcctatccaatttcaaa
aaag

SEQ ID NO: 523

gaaactgtatgggtagcttttttgtttgttttttgttttgtttttgtttt
tgtttttgtttttagttgtaggtcgcagcggggaaattttttgcgactgt
acacatagctgcagcattaaaaacttaaaaaaattgttaaaaaaanaaaa
aaagggaaaacatttcaaaaaaaaaaaaanngataaacagttacaccttg
ttttcaatgtgtggctgagtgcctcgattttttcatgtttttggtgtatt
tctgatttgtagaagtgtccaaacaggttgtgtgctggagttccttcaag
acaaaaacaaacccagcttggtcaaggccattacctgtttcccatctgta
gttattcg

SEQ ID NO: 524

cgcccaccaccatgagctggagtggggatgacaagacttgtgttcctcaa
ctttcttgggtttctttcaggatttttcttctcacagctccaagcacgtg
tcccgtgcctccccactcctcttaccacccctctctctgacactttttgt
gttgggtcctcagccaacactcaaggggaaacctgtagtgacagtgtgcc
ctggtcatccttaaaataacctgcatctcccctgtcctggtgtgggagta
agctgacagtttctctgcaggtcctgtcaactttagcatgctatgtcttt
accatttttgctctcttgcagttttttgctttgtcttatgcttctatgga
taatgctatataatcattatctttttatctttctgttattattgttttaa
aggagagcatcctaagttaataggaaccaaaaaataatgatgggcagaag
ggggggaatagccacaggggacaaaccttaaggcattataagtgacctta
tttctgcttttctgagctaagaatggtgctgatggtaaagtttgagactt
ttgccacacacaa

SEQ ID NO: 525

tttgtcatatgaccttctgaagcagccacaacttagataatgtcagaact
aaggtganttttttttttttaattttgaaagcccagccaaaatgaggtgt
gaatttgtcatactgttacattgaaattggtaacaaaatatatcccctcc
catttggacttttagggtaaatgaaaattttattgtattttaaagtagtt
tctaagtgttagcaagactgactataattccagtttctgttttctatgga
cagacctgataaactggagaccctaaagcaggaatacccaaattatagtg
tcaggattttagctgtaccagaggcctttatgtgctacacataatttgta
taaaattttatatgtgcagattgggtacataaacagttctccatt

SEQ ID NO: 526

gtgctacagatactacatttcaaagagttggcattttccctttggccact
caagcagcatttgatgtatctaaagnaacaaagtcattgtttatttttta
aaaaattatatgcagttgtacaagatactacattccattgaaatgttggc
tatgtcctaaccaggcaaccagataacaaaaacattttgagtcttttatc
taggtagttctaattattcagctacttagtttaacaaaggaaaatatcct
gacttctctcatttcatttgtagacttttcattgtataggcacaaccaaa
gagtcagactggtttaaaactccagaaggaaaaaaagtatcccacacagt
ggatgttgtttctaagaatgctacaaaatcctgacatctcagacatctca
atgttaaaggaagaaaaaaaataccttttcatttcaaagaactaatatac
tttgatattgtgtaaaccttactcaagtttattgtcaagctttaactgcc
tttttagaactttttaaaatttcgagcccacaaatctat

SEQ ID NO: 527

ctgcccgagctggtgcattacagagaggagaaacacatcttccctagagg
gttcctgtagacctagggaggaccttatctgtgcgtgaaacacaccaggc
tgtgggcctcaaggacttgaaagcatccatgtgtggactcaagtccttac
ctcttccggagatgtagcaaaacgcatggagtgtgtattgttcccagtga
cacttcagagagctggtagttagtagcatgttgagccaggcctgggtctg
tgtctcttttctctttctccttagtcttctcatagcattaactaatctat
tgggttcattattggaattaacctggtgctggatattttcaaattgtatc
tagtgcagctgattttaacaataactactgtgttcctggcaatagtgtgt
tctg

SEQ ID NO: 528

gagacttcattgtatgacttcagttaaaatactattttgtatgcattctt
tattcacttaagaagcttgtctgcaataataaagccacgtcatgtcttct
ttngggagggagagagtcgatggcaggagggggttttgggtgggccactg
aaaaggggtaccgaataggttgtgtgatgaaattctgtgtcttggaactg
gaattgagtttcgatgttgatgaactgattcaaccaggtgttgaaggcac
gacagccactgctctacgaaaaggcagagtacgtttttcccttctggttg
taacctggttgagagcttcccctttatcagattggcagctaaacagttgt
attagataatccttaaatctgacatccagcctgttacgctctagggctcg
ctgcttggcctgcgtttgctttttattgtgtatccgttcccctcctacgg
tgtgctcctgaatgaaggtttctatgtaagcagatgatgattttacctgt
caataccagcactgtattactaacatgca

SEQ ID NO: 529

tgcccttccaggtgggtgtgggacacctgggagaaggtctccaagggagg
gtgcagccctcttgcccgcacccctccctgcttgcacacttccccatctt
tgatccttctgagctccacctctggtggctcctcctaggaaaccagctcg
tgggctgggaatgggggagagaagggaaaagntccccaagaccccctggg
gtgggatntgagctcccacctcccttnccacntantgcactttccccctt
cccgccttccaaaacctgcttcdttcagtttgtaaagtcggtgattatat
ttttgggggctttccttttattttttaaatgtaaaatttatttatattcc
gtatttaaagttgtaaaaaaaaataaccacaaaacaaaaccaaaaaaaaa
aaaaaacttctcctcctgcagccgggagcggccggcctgcctccctgcgc
acccgcagcctcccccgctgcctccctagggctcccctccggccgccagc
gcccatttttcattccctagatagag

SEQ ID NO: 530

tgatgaatcccacaaaagtcagcaccttctacagaacagatgccctgatc
accaaggacttggtactgatttagagagaagagagcagctcctagcagca
tcaacatctatttgtcgcttatttgccctgc

SEQ ID NO: 531

gaagccggcaggtttcggacaacacaggtcctggtcggacaccacatccc
tccccatccgcaggatgtggaaaagcagatgcaggagtttgtacagtggc
tcaactccgaggaagccatgaacctgcacccagtggagtttgcagcctta
gcccattataaactcgtttacatccaccctttcattgatggcaacgggag
gacctcccgtctgctcatgaacctcatcctcatgcaggcgggctacccgc
ccatcaccatccgcaaggagcagcggtccgactactaccacgtgttggaa
gctgccaacgagggcgacgtgaggcctttcattcgcttcatcgccaagtg
tactgagaccaccctggacaccctgctttttgccacaactgagtactcgg
tggcactgccagaagcccaacccaaccactctgggttcaaggagacgctt
cctgtgaagcccta

SEQ ID NO: 532

ccaaagtgtttgcttctccctttctgcggccttcgccagcccaggctcgg
ctgccacccagtggnacagaaccgaggagctgccattnncccccatangg
gnnagtgtcttgttncnnnnnnnnnnnnnnntcnttgcttctgncagctc
cttcccctaggagggaagggtggggtggaactgggcacatgccagcacc

SEQ ID NO: 533

gccacttgtcttgaaaactgtgcaactttttaaagtaaattattaagcag
actggaaaagtgatgtattttcatagtgacctgtgtttcacttaatgttt
cttagagccaagtgtcttttaaacattattttttatttctgatttcataa
ttcagaactaaatttttcatagaagtgttgagccatgctacagttagtct
tgtcccaattaaaatactatgcagtatctcttacatcagtagcatttttc
taaaaccttagtcatcagatatgcttactaaatcttcagcatagaaggaa
gtgtgtttgcctaaaacaatctaaaacaattcccttctttttcatcccag
accaatggcattattaggtcttaaagtagttactcccttctcgtgtttgc
ttaaaatatgtgaagttttccttgctatttcaataacagatggtgctgct
aattcccaacatt

SEQ ID NO: 534

ttgcatttggattggggtccctctaaaatttaatgcatgatagacacata
tgagggggaatagtctagatggctcctctcagtactttggaggcccctat
gtagtccgtgctgacagctgctcctagagggaggggcctaggcctcagcc
agagaagctataaattcctctttgctttgctttctgctcagcttctcctg
tgtgattgacagctttgctgctgaaggctcattttaatttattaattgct
ttgagcacaactttaagaggacataatgggggcctggccatccacaagtg
gtggtaaccctggtggttgctgttttcctcccttctgctactggcaaaag
gatctttgtggccaaggagctgctatagcctggggtggggtcatgccctc
ctctcccattgtccctctgccccatcctccagcagggaaaatgcagcagg
gatgccctggaggtggctgagcccctgtctagagagggaggcaagccctg
ttgacacaggtctttcctaaggctgcaaggtttaggctggtggccc

SEQ ID NO: 535

gggggaaaacgaccctgtattgcagaggattgtagacattctgtatgcca
cagatgaaggctttgtgatacctgatgaagggggcccacaggaggagcaa
gaagagtattaacagcctggaccagcagagcaacatcggaattcttcact
ccaaatcatgtgcttaactgtaaaatactcccttttgttatccttagagg
actcactggtttcttttcataagcaaaaagtacctcttcttaaagtgcac
tttgcagacgtttcactccttttccaataagtttgagttaggagctttta
ccttgtagcagagcagtattaacanctagttggttcacctggaaaacaga
gaggctgaccgtggggctcaccatgcggatgcgggtcacactgaatgctg
gagagatgttatgtaatatgctgaggtggcgacctcagtggagaaatg

SEQ ID NO: 536

agctttcttcaccttatatatgttcttccactgtgactttttagttgaag
actagtaaattaacttttagttagaagatgcctactgcttttgttgttta
ttttaatcagcagagcacagagacacataaaaactctgggaaatgactag
gataaaaatatcagtatgtatctgttttagatattttgagttttgctttt
tttatgccttgaatattttatttcaaaaagtatctgaagcaaattctcag
actgaactacttcttagacctcactgtaagaatattttattcaatgtctc
atttatgatagatttgcaagctgctcatttttgaacagctttttgcatgg
gataggagcatgtctattctaacacatcagcttattcaaaagcaagaatt
ttaaaaataagataaatgtaaagttgttttataaacgatcctgttaatta
aaccacagacaccatatatccttctgca

SEQ ID NO: 537

tacccaggtgattatatttgttgatctaataanatggaaggtttgtttta
tatgaattttcaaaaagatgtctctttacactttttgttaccttgtagac
tcttattgataaatgcaactacttattaaaattgttcacttttngtcttt
tgatcagatgcctttagtcaggtaagtttaagggaaaatacgcagtttaa
tgttttggtacatataattatgtctgccaaagaaacctttgattgtatca
tattgcctatttagtagtgcatagggttcagagtacatgataaaggatca
aaagctttgcattgataagtgtctcataatatttgctgtgatt

SEQ ID NO: 538

cacttattcttttcagtaacctgctagtgcacaggctgtactttaggtac
ttaaaatatgcactagaataaatttgcaaggccctaaaatatcactgtta
tttttggagtaattcagtataggttcgtttaaaagagatttttataactt
cagacatgcatcagtaggaaataacttgagaaattcatatggttatgtta
caaattcatattctgttactacagtaaacgttaagagttttaaacagtta
agattgtacaatttttcttcttttctatattacaagggccccagtgttaa
tgtcttagattttcagtatttgaacttatttttttaaattctgtcattga
gataagaataattcaggtagcatctgaaattttaatgaatgtataattgg
catatcatggaaaattaaccagaaagtatcagttcttaaaagttatgcct
ag

SEQ ID NO: 539

gaagccacaaagatgccacatgttagtatatcagtgagaggtgactccac
agtgctctctggagaagcaatatgagtgactgaagagtggggccttttgc
ttttgcctggatataggggtgctcttctactgtaattgggtgtggaaaaa
ctctggctttatggtattccattaggttcttttcatttaaagtagtctta
aaatcaaagtatccaatattttaaagccacaaagtagattacataattag
cagagattttagtcagtaaaatgttagaaatcaaactataagaaaattca
agtcctttattttgtgtcttgggtatatgtcattattttaaattccacac
tcccttatttaatcactttggtaagtgcctttgatgttttgaaatgtata
gtgggagatgagcaaatgtaaatgtcatgtgccctgttccctagcttctc
aattcctcataaccatttttaccagtgttgcaaagtttagacctttgtgt
taatatcagaagtgtatttgtagcccctccatagtgaacaatga

SEQ ID NO: 540

ttcttcagccctagatggtgctcgccagacctcctctcaatgctcatcac
acacagggctattcctttcctccaatgaaccaaaccgcctcccgcccacc
tccaggtcccagtcctctgttccctttgcctggtccacccttgccctccc
tgggtcgcagacgaggtcggcctcgtcattccccgcagaccgccgcgcgt
ccctcttgtgcggttcaccacagttgtatttaagtgatcgtgtgagtcgt
cgttaaatgcctgtctccccgcggatcatgggctcctcgaggacagggac
tggcctgtctgtccactgctgtaaccccgcgccggcatagggacctaagg
cccactggagggcgctcatcaagtagctgctggatgttgacgaaggaagc
ggcggcgcagctcagggatctccgagtcaggacggtcggcc

SEQ ID NO: 541

aacaatacctgcttttacaccaagaatggacatagtttaggtattgcttt
cactgacctaccgccaaatttgtatcctgttagtcctcgaccttttagta
gtccaagtatgagccccagccatggaatgaatatccacaatttagcatca
ggcaaaggaagcaccgcacatttttcaggttttgaaagttgtagtaatgg
tgtaatatcaaataaagcacatcaatcatattgccatagtaataaacacc
agtcatccaactttcaatgtaccagaactaaacagtataaatatgtcaag
atcacagcaagttaataacttcaccagtaatgatgtagacatggaaatag
atcactactccaatggagttggagaaacttcatccaatggtttcctaaat
ggtagctctaaacatgaccacgaaatggaagattgtgacaccgaaatgga
agttgattcaagtcagttgagacgtcagttgtgtggaggaagtcaggccg
ccatagaaagaatgatccactttggacgagagctgcaa

SEQ ID NO: 542

cacttccagcccatgtacactagtggcccacgaccaaggggtcttcattt
ccatgaaaaagggactccaagaggcagtggtggctgtggcccccaacttt
ggtgctccagggtgggccagctgcttgtgggggcacctgggaggtcaaag
gtctccaccacatcaacctattttgttttaccctttttctgtgcattgtt
tttttttttcctcctaaaaggaatatcacggttttttgaaacactcagtg
ggggacattttggtgaagatgcaatatttttatgtcatgtgatgctcttt
cctcacttgaccttggccgctttgtcctaacagtccacagtcctgccccg
acccaccccatcccttttctctggcactccagtcccaggccttgggcctg
aactactggaaaaggtctggcggctggggaggagtgccagcaa

SEQ ID NO: 543

acttcgctacttggctagagttgcaactacagctgggttatatggctcta
atctgatggaacatactgagattgatcactggttggagttcagtgctaca
aaattatcttcatgtgattcctttacttctacaattaatgaactcaatca
ttgcctgtctctgagaacatacttagttggaaactccttgagtttagcag
atttatgtgtttgggccaccctaaaaggaaatgctgcctggcaagaacag
ttgaaacagaagaaagctccagttcatgtaaaacgttggtttggctttct
tgaagcccagcaggccttccagtcagtaggtaccaagtgggatgtttcaa
caaccaaagctcgagtggcacctgagaaaaagcaagatgttgggaaattt
gttgagcttccaggtgcggagatgggaaaggttaccgtcagatttcctcc
agaggccagtggttacttacacattgggcatgcaaaagctgctcttctga
accagcactaccaggt

SEQ ID NO: 544

ccctcacacgtgcgcaggaagatcatgtcatccccgctctccaaggagct
gcggcagaagtacaatgtccgctccatgcccatccgcaaggacgacgagg
tccaggtagttcgaggacactacaaaggtcagcaaattggcaaggtagtc
caggtgtacagaaagaaatatgtcatctacatcgagcgggtgcagcgtga
gaaggccaacggcacaactgtccacgtgggcattcacccaagcaaggtgg
ttatcaccaggctaaaactggacaaggatcggaaaaaaattcttgaacgc
aaagccaagtctcgacaagttggaaaagagaaaggcaaatataaagaaga
acttattgagaaaatgcaggaataaatagaacctgttgtgcaaccacggt
ttaaccggagattttgaggctagggtgtgtttctttcgaacttttcggaa
tgtctggaacatttcatttcctgttttgttacctgtgcctctgtaaatct

SEQ ID NO: 545

tgcaggcactcagaatggtccagcgtttgacataccgacgtaggctttcc
tacaatacagcctctaacaaaactaggctgtcccgaacccctggtaatag
aattgtttacctttataccaagaaggttgggaaagcaccaaaatctgcat
gtggtgtgtgcccaggcaaacttcgaggggttcgtcctgtaagacctaaa
gttcttatgagattgtccaaaacaaagaaacatgtcagcagggcctatgg
tggttccatgtgtgctaaatgtgttcgtgacaggatcaagcgtgctttcc
tta

SEQ ID NO: 546

cgcagaatggctcccgcaaagaagggtggcgagaagaaaaagggccgttc
tgccatcaacgaagtggtaacccgagaatacaccatcaacattcacaagc
gcatccatggagtgggcttcaagaagcgtgcacctcgggcactcaaagag
attcggaaatttgccatgaaggagatgggaactccagatgtgcgcattga
caccaggctcaacaaagctgtctgggccaaaggaataaggaatgtgccat
accgaatccgtgtgcggctgtccagaaaacgtaatgaggatgaagattca
ccaaataagctatatactttggttacctatgtacctgttaccactt

SEQ ID NO: 547

tgttctgctgcttagccagttcatccggcctcatggaggcatgctgcccc
gaaagatcacaggcctatgccaggaagaacaccgcaagatcgaggagtgt
gtgaagatggcccaccgagcaggtctattaccaaatcacaggcctcggct
tcctgaaggagttgttccgaagagcaaaccccaactcaaccggtacctga
cgcgctgggctcctggctccgtcaagcccatctacaaaaaaggcccccgc
tggaacagggtgcgcatgcccgtggggtcaccccttctgagggacaatgt
ctgctactcaagaacaccttggaagctgtatcactgacagagagcagtgc
ttccagagttcctcctgcacctgtgctggggagtaggaggcccactcaca
agcccttggccacaactatactcctgtcccaccccaccacgatggcctgg
tccctccaacatgcatggacaggggacagtgggactaacttcagtaccct
tggcctgcacagtagcaatgc

SEQ ID NO: 548

cctatggccgtgggcctcaacaagggccacaaagtgaccaagaacgtgag
caagcccaggcacagccgacaccgcgggcgtctgaccaaacacaccaagt
tcgtgcgggacatgattcgggaggtgtgtggctttgccccgtacgagcgg
cgcgccatggagttactgaaggtctccaaggacaaacgggccctcaaatt
tatcaagaaaagggtggggacgcacatccgc

SEQ ID NO: 549

tcaaaagtaagttctccatcccataaagccatttaaattcattagaaaaa
tgtccttacctcttaaaatgtgaattcatctgttaagctaggggtgacac
acgtcattgtaccctttttaaattgttggtgtgggaagatgctaaagaat
gcaaaactgatccatatctgggatgtaaaaaggttgtggaaaatagaatg
tccagacccgtctacaaaaggtttttagagttgaaatatgaaatgtgatg
tgggtatggaaattgactgttacttcctttacagatctacagacagt

SEQ ID NO: 550

gccgcctaaggacgacaagaagaagaaggacgctggaaagtcggccaaga
aagacaaagacccagtgaacaaatccgggggcaaggccaaaaagaagaag
tggtccaaaggcaaagttcgggacaagctcaataacttagtcttgtttga
caaagctacctatgataaactctgtaaggaagttcccaactataaactta
taaccccagctgtggtctctgagagactgaagattcgaggctccctggcc
agggcagcccttcaggagctccttagtaaaggacttatcaaactggtttc
aaagcacagagctcaagtaatttacaccagaaataccaagggtggagatg
ctccagctgctggtgaagatgcatgaataggtccaaccagctgta

SEQ ID NO: 551

cccccaactatgaccatgtggtcctgggcggtggtcaggaagccatggat
gtaaccacaacctccaccaggattggcaagtttgaggccaggttcttcca
tttggcctttgaagaagagtttggaagagtcaagggtcactttggaccta
tcaacagtgttgccttccatcctgatggcaagagctacagcagcggcggc
gaagatggttacgtccgtatccattacttcgacccacagtacttcgaatt
tga

SEQ ID NO: 552

ggtgagcgaagctgggacaggtttctgcttcaacaccaagagaaaccgac
tgcgggaaaaactgactcttttgcattatgatccagttgtgaaacaaaga
gtcctcttcgtggaaaagaaaaaaatacgctccctttaaacggtggattg
aaaatgactttgatttataaagagaagactgagggcggggatactgattc
agaaatcctgtagcgtgtaataaaagaagaggaaatggcatggaatcact
gcctcctgtgatttgaaggccattgtgaaggaaaacaatgcagtgaaaga
aagttcttcatattaggacagatatcattgcatcacatttatttatcttt

SEQ ID NO: 553

gtcgctctttgtataacaccaagcagatgctgcctgcagagggtgtgaag
gagctgtgtctgctgctgcttaaccagtccctcctgcttccatctctgaa
acttctcctcgagagccgagatgagcatctgcacgagatggcactggagc
aaatcacggcagtcactacggtgaatgattccaattgtgaccaagaactt
ctttccctgctcctggatgccaagctgctggtgaagtgtgtctccactcc
cttctatccacgtattgttgaccacctcttggctagcctccagcaagggc
gctgggatgcagaggagctgggcagacacctgcgggaggccggccatgaa
gccgaagccgggtctctccttctggccgtgagggggactcaccaggcctt
cagaaccttcagtacagccctccgcgcagcacagcactgggtgttgaagc
cacctgtggccctgctccttagcagaaaaagcatctggagttgaatgctg
ttcccagaagcaacatgtgtatctgccgattgttctccatggttccaaca
a

SEQ ID NO: 554

ggctaagcaagcatctaaaaagactgcaatggctgctgctaaggcaccta
caaaggcagcacctaagcnaaagattgtgaagcctgtgaaagtttcagct
ccccgagttggtggaaaacgctaaactggcagatta

SEQ ID NO: 555

cccagaacctaacatccttcaagaattccaccaagtcctgggtgggcttc
tctggtggccagcaccatacagtctgcatggattcggaaggaaaagcata
cagcctgggccgggctgagtatgggcggctgggccttggagagggtgctg
aggagaagagcatacccaccctcatctccaggctgcctgctgtctcctcg
gtggcttgtggggcctctgtggggtatgctgtgaccaaggatggtcgtgt
tttcgcctggggcatgggcaccaactaccagctgggcacagggcaggatg
aggacgcctggagccctgtggagatgatgggcaaacagctggagaaccgt
gtggtcttatctgtgtccagcgggggccagcatacagtcttattagtcaa
ggacaaagaacagagctgatgaagcctctgagggcctggcttctgtcctg
cacaacctccctcacagaacagggaagcagtgacagctgcagatggcagc
gggcctct

SEQ ID NO: 556

gtaagatgtctctagcactgctcaaagggcaaattttaaaacttcagtct
gggtgaaagatttgctagttttacagaaagatttgctatcttaaactcaa
gctggtttttctgttctcatgtaagtgactgggatgctgtcttatgaatt
cttccaaggtcatgtttgtgaaataaacattacatgagagctttcctgtc
atctacactatatgttgtctggagtgttgaacaaatttattttagtttct
aagttgtaatctatcctcatatggtctatacgattttgaatgtgtgccac
tacatactgagatgataatgctgtacaattttaagtggtagcagtttctg
tatgcagta

SEQ ID NO: 557

aagccactcagttgatgctcacactgctgaagtgaactgcctttctttca
atccttatagtgagttcattcttgccacaggatcagctgacaagactgtt
gccttgtgggatctgagaaatctgaaacttaagttgcattcctttgagtc
acataaggatgaaatattccaggttcagtggtcacctcacaatgagacta
ttttagcttccagtggtactgatcgcagactgaatgtctgggatttaagt
aaaattggagaggaacaatccccagaagatgcagaagacgggccaccaga
gttgttgtttattcatggtggtcatactgccaagatatctgatttctcct
ggaatcccaatgaaccttgggtgatttgttctgtatcagaagacaatatc
atgcaagtgtggcaaatggagttagtccttgaccactagtttgatgccat
ctccattttgggtgacctgtttcaccagcaggc

SEQ ID NO: 558

aggccaagacccatgttcttgacattgagcagcgactacaaggtgtaatc
aagactcgaaatagagtgacaggactgccgttatctattgaaggacatgt
gcattaccttatacaggaagctactgatgaaaacttactatgccagatgt
atcttggttggactccatatatgtgaaatgaaattatgtaaaagaatatg
ttaataatctaaaagtaatgcatttggtatgaatctgtggttgtatctgt
tcaattctaaagtacaacataaatttacgttctcagcaactgttatttct
ctctg

SEQ ID NO: 559

gtacgtgggggtctggctgagagtacagggctgctggcggtcagtgatga
gatcctcgaggtcaatggcattgaagtagccgggaagaccttggaccaag
tgacggacatgatggttgccaacagccataacctcattgtcactgtcaag
cccgccaaccagcgcaataacgtggtgcgaggggcatctgggcgtttgac
aggtcctccctctgcagggcctgggcctgctgagcctgatagtgacgatg
acagcagtgacctggtcattgagaaccgccagcctcccagttccaatggg
ctgtctcaggggcccccgtgctgggacctgcaccctggctgccgacatcc
tggtacccgcagctctctgccctccctggatgaccaggagcaggccagtt
ctggctgggggagtcgcattcgaggagatggtagtggcttcagcctctga
cagtcaggatgaagccccatgccactccacactgctgggacatggcaggg
acttcacagtgggggtttttagctggctcaca

SEQ ID NO: 560

atatgcttactgtgcacctagagcttttttataacaacgtctttttgttt
gtttgnttttggattctttaaatatatattattctcatttagtgccctct
ttagccagaatctcattactgcttcatttttgtaataacatttaatttag
atattttccatatattggcactgctaaaatagaatatagcatctttcata
tggtaggaaccaacaaggaaactttcctttaactccctttttacacttta
tggtaagtagcagggggggaaatgcatttatagatcatttctaggcaaaa
ttgtgaagctaatgaccaacctgtttctacctatatgcagtctctttatt
ttactagaaatgggaatcatggcctcttgaagagaaaaaagtcaccattc
tgcatttagctgtattcatat

SEQ ID NO: 561

gcacaagctgtgacaggctccatccagcccctcagtgctcaggccctggc
tggaagtctgagctctcaacaggtgacaggaacaactttgcaagtccctg
gtcaagtggccattcaacagatttccccaggtggccaacagcagaagcaa
ggccagtctgtaaccagcagtagtaatagacccaggaagaccagctcttt
atcgcttttctttagaaaggtataccatttagcagctgtccgccttcggg
atctctgtgccaaactagatatttcagatgaantgaggaaaaaaatctgg
acctgctttgaattctccataattcagtgtcctgaacttatgatggacag
acatctggaccagttattaatgtgtgccatttatgtgatggcaaaggtca
caaaagaagataagtccttccagaacattatgcgttgttataggactcag
ccgcaggcccggagccaggtgtataga

SEQ ID NO: 562

catcatccccattccgaagggtcagggaggaggaaattgaggtggattca
cgagttgcggacaactcctttgatgccaagcgaggtgcagccggagactg
gggagagcgagccaatcaggttttgaagttcaccaaaggcaagtcctttc
ggcatgagaaaaccaagaagaagcggggcagctaccggggaggctcaatc
tctgtccaggtcaattctattaagtttgacagcgagtgacctgaggccat
cttcggtgaagcaagggtgatgatcggagactacttactttctccagtgg
acctgggaaccctcaggtctctaggtgagggtcttgatgaggacagaagt
ttagagtaggtcctaagactttacagtgtaacatcctctctggtcc

SEQ ID NO: 563

gtttgatcatccagccaagattgccaagagtactaaatcctcttccctaa
atttctccttcccttcacttcctacaatgggtcagatgcctgggcatagc
tcagacacaagtggcctttccttttcacagcccagctgtaaaactcgtgt
ccctcattcgaaactggataaagggcccactggggccaatggtcacaaca
cgacccagacaatagactatcaagacactgtgaatatgcttcactccctg
ctcagtgcccagggtgttcagcccactcagcccactgcatttgaatttgt
tcgtccttatagtgactatctgaatcctcggtctggtggaatctcctcga
ga

SEQ ID NO: 564

atctgtttggtttgacacccagcctcttccctggccctccccagagaact
ttgggtacctggtgggtctaggcagggtctgagctgggacaggttctggt
aaatgccaagtatgggggcatctgggcccagggcagctggggagggggtc
agagtgacatgggacactccttttctgttcctcagttgtcgccctcacga
gaggaaggagctcttagttacccttttgtgttgcccttctttccatcaag
gggaatgttctcagcatagagctttctccgcagcatcctgcctgcgtgga
ctggctgctaatggagagctccctggggttgtcctggctctggggagaga
gacggagcctttagtacagctatctgctggctctaaaccttctacgcctt
tgggccgagcactgaatgtcttgtact

1. A method for predicting distant metastasis of lymph node negative primary breast cancer comprising the steps of:
a) obtaining breast cancer cells;
b) isolating nucleic acid and/or protein from the cells; and
c) analyzing the nucleic acid and/or protein to determine the presence, expression level or status of a Biomarker selected from the pathways in Table 4.

2. The method according to claim 1 wherein gene expression is analyzed by determining the expression of the biomarkers corresponding to those listed in Table 1, Table 5 or Table 6.

3. A composition comprising an oligonucleotide related to the markers listed in Table 1, Table 5 or Table 6.

4. A kit comprising biomarker detection agents for performing the method according to claim 1.

5. An article comprising biomarker detection agents for performing the method according to claim 1.
